(19) United States
(12) Patent Application Publication
(10) Pub. No.: US 2013/0143761 A1
(43) Pub. Date: Jun. 6, 2013

(54) PRODUCT AND METHOD

(71) Applicants: Praveen SHARMA, Oslo (NO); Narinder Singh SAHNI, Oslo (NO); Anders LONNEBORG, Aas (NO)

(72) Inventors: Praveen SHARMA, Oslo (NO); Narinder Singh SAHNI, Oslo (NO); Anders LONNEBORG, Aas (NO)

(73) Assignee: DIAGENIC AS, Oslo (NO)

(21) Appl. No.: 13/735,740

(22) Filed: Jan. 7, 2013

Related U.S. Application Data
(63) Continuation of application No. 10/535,414, filed on May 1, 2006.

(30) Foreign Application Priority Data
Nov. 21, 2002 (GB) 0227238.3

(51) Int. Cl. C12Q 1/68

(52) U.S. Cl. CPC C12Q 1/6876; G06F 19/20
USPC 506/9; 506/16; 530/350; 530/389.1; 702/19

(57) ABSTRACT

The present invention relates to oligonucleotide probes, for use in assessing gene transcript levels in a cell, which may be used in analytical techniques, particularly diagnosis techniques and kits containing the same.

This application is a 371 of PCT/GB2003/005102, filed Nov. 21, 2003, the disclosure of which is incorporated herein by reference.

### PRODUCT AND METHOD

A Sequence Listing on a single CD-ROM was filed with this application (file name: Q87920.ST25.txt). The Sequence Listing contains each of the polynucleotide and polypeptide sequences disclosed herein. The Sequence Listing is incorporated herein by reference.

The present invention relates to oligonucleotide probes, for use in assessing gene transcript levels in a cell, which may be used in analytical techniques, particularly diagnostic techniques. Conveniently the probes are provided in kit form. Different sets of probes may be used in techniques to prepare gene expression patterns and identify, diagnose or monitor different states, such as diseases, conditions or stages thereof. Also provided are methods of identifying suitable probes and their use in methods of the invention.

The identification of quick and easy methods of sample analysis for, for example, diagnostic applications, remains the goal of many researchers. End users seek methods which are cost effective, produce statistically significant results and which may be implemented routinely without the need for highly skilled individuals.

The analysis of gene expression within cells has been used to provide information on the state of those cells and importantly the state of the individual from which the cells are derived. The relative expression of various genes in a cell has been identified as reflecting a particular state within a body. For example, cancer cells are known to exhibit altered expression of various proteins and the transcripts or the expressed proteins may therefore be used as markers of that disease state.

Thus biopsy tissue may be analysed for the presence of these markers and cells originating from the site of the disease may be identified in other tissues or fluids of the body by the presence of the markers. Furthermore, products of the altered expression may be released into the blood stream and these products may be analysed. In addition cells which have contacted disease cells may be affected by their direct contact with those cells resulting in altered gene expression and their expression or products of expression may be similarly analysed.

However, there are some limitations with these methods. For example, the use of specific tumour markers for identifying cancer suffers from a variety of defects, such as lack of specificity or sensitivity, association of the marker with disease states besides the specific type of cancer, and difficulty of detection in asymptomatic individuals.

In addition to the analysis of one or two marker transcripts or proteins, more recently, gene expression patterns have been analysed. Most of the work involving large-scale gene expression analysis with implications in disease diagnosis has involved clinical samples originating from diseased tissues or cells. For example, several recent publications, which demonstrate that gene expression data can be used to distinguish between similar cancer types, have used clinical samples from diseased tissues or cells (Alon et al. 1999, PNAS, 96, p 6745-6750; Golub et al. 1999, Science, 286, p 531-537; Alizadeh et al, 2000, Nature, 403, p 503-511; Bittner et al., 2000, Nature, 406, p 536-540).

However, these methods have relied on analysis of a sample containing diseased cells or products of those cells or cells which have been contacted by disease cells. Analysis of such samples relies on knowledge of the presence of a disease and its location, which may be difficult in asymptomatic patients. Furthermore, samples can not always be taken from the disease site, e.g. in diseases of the brain.

In a finding of great significance, the present inventors identified the previously untapped potential of all cells within a body to provide information relating to the state of the organism from which the cells were derived. WO98/49342 describes the analysis of the gene expression of cells distant from the site of disease, e.g. peripheral blood collected distant from a cancer site.

This finding is based on the premise that the different parts of an organism's body exist in dynamic interaction with each other. When a disease affects one part of the body, other parts of the body are also affected. The interaction results from a wide spectrum of biochemical signals that are released from the diseased area, affecting other areas in the body. Although, the nature of the biochemical and physiological changes induced by the released signals can vary in the different body parts, the changes can be measured at the level of gene expression and used for diagnostic purposes.

The physiological state of a cell in an organism is determined by the pattern with which genes are expressed in it. The pattern depends upon the internal and external biological stimuli to which said cell is exposed, and any change either in the extent or in the nature of these stimuli can lead to a change in the pattern with which the different genes are expressed in the cell. There is a growing understanding that by analysing the systemic changes in gene expression patterns in cells in biological samples, it is possible to provide information on the type and nature of the biological stimuli that are acting on them. Thus, for example, by monitoring the expression of a large number of genes in cells in a test sample, it is possible to determine whether their genes are expressed with a pattern characteristic for a particular disease, condition or stage thereof. Measuring changes in gene activities in cells, e.g. from tissue or body fluids is therefore emerging as a powerful tool for disease diagnosis.

Such methods have various advantages. Often, obtaining clinical samples from certain areas in the body that is diseased can be difficult and may involve undesirable invasions in the body, for example biopsy is often used to obtain samples for cancer. In some cases, such as in Alzheimer's disease the diseased brain specimen can only be obtained post-mortem. Furthermore, the tissue specimens which are obtained are often heterogeneous and may contain a mixture of both diseased and non-diseased cells, making the analysis of generated gene expression data both complex and difficult.

It has been suggested that a pool of tumour tissues that appear to be pathogenetically homogeneous with respect to morphological appearances of the tumour may well be highly heterogeneous at the molecular level (Alizadeh, 2000, supra), and in fact might contain tumours representing essentially different diseases (Alizadeh, 2000, supra; Golub, 1999, supra). For the purpose of identifying a disease, condition, or a stage thereof, any method that does not require clinical samples to originate directly from diseased tissues or cells is highly desirable since clinical samples representing a homogeneous mixture of cell types can be obtained from an easily accessible region in the body.

We have now identified a set of probes of surprising utility for identifying one or more diseases.

Thus, we now describe probes and sets of probes derived from cells which are not disease cells and which have not contacted disease cells, which correspond to genes which exhibit altered expression in normal versus disease individuals, for use in methods of identifying, diagnosing or monitoring certain conditions, particularly diseases or stages thereof.

Thus the invention provides a set of oligonucleotide probes which correspond to genes in a cell whose expression is affected in a pattern characteristic of a particular disease, condition or stage thereof, wherein said genes are systemically affected by said disease, condition or stage thereof. Preferably said genes are metabolic or house-keeping genes and preferably are constitutively moderately or highly expressed. Preferably the genes are moderately or highly expressed in the cells of the sample but not in cells from disease cells or in cells having contacted such disease cells.

Such probes, particularly when isolated from cells distant to the site of disease, do not rely on the development of disease to clinically recognizable levels and allow detection of a disease or condition or stage thereof very early after the onset of said disease or condition, even years before other subjective or objective symptoms appear.

As used herein “systemically” affected genes refers to genes whose expression is affected in the body without direct contact with a disease cell or disease site and the cells under investigation are not disease cells.

“Contact” as referred to herein refers to cells coming into close proximity with one another such that the direct effect of one cell on the other may be observed, e.g. an immune response, wherein these responses are not mediated by secondary molecules released from the first cell over a large distance to affect the second cell. Preferably contact refers to physical contact, or contact that is as close as is sterically possible, conveniently, cells which contact one another are found in the same unit volume, for example within 1 cm³.

A “disease cell” is a cell manifesting phenotypic changes and is present at the disease site at some time during its life-span, e.g. a tumour cell at the tumour site or which has disseminated from the tumour, or a brain cell in the case of brain disorders such as Alzheimer's disease.

“Metabolic” or “house-keeping” genes refer to those genes responsible for expressing products involved in cell division and maintenance, e.g. non-immune function related genes.

“Moderately or highly” expressed genes refers to those present in resting cells in a copy number of more than 30-100 copies/cell (assuming an average 3×10⁵ mRNA molecules in a cell).

Specific probes having the above described properties are provided herein.

Thus in one aspect, the present invention provides a set of oligonucleotide probes, wherein said set comprises at least 10 oligonucleotides selected from:
an oligonucleotide as described in Table 1 or derived from a sequence described in Table 1, or an oligonucleotide with a complementary sequence, or a functionally equivalent oligonucleotide.

“Table 1” as referred to herein refers to Table 1a and/or Table 1b. Table 1b contains reference to additional clones and sequences as disclosed herein. Similarly Tables 2 and 4 comprise 2 parts, a and b.

The invention also provides one or more oligonucleotide probes, wherein each oligonucleotide probe is selected from the oligonucleotides listed in Table 1, or derived from a sequence described in Table 1, or a complementary sequence thereof. The use of such probes in products and methods of the invention, form further aspects of the invention. As referred to herein an “oligonucleotide” is a nucleic acid molecule having at least 6 monomers in the polymeric structure, ie. nucleotides or modified forms thereof. The nucleic acid molecule may be DNA, RNA or PNA (peptide nucleic acid) or hybrids thereof or modified versions thereof, e.g. chemically modified forms, e.g. LNA (Locked Nucleic acid), by methylation or made up of modified or non-natural bases during synthesis, providing they retain their ability to bind to complementary sequences. Such oligonucleotides are used in accordance with the invention to probe target sequences and are thus referred to herein also as oligonucleotide probes or simply as probes.

An “oligonucleotide derived from a sequence described in Table 1” (or any other table) refers to a part of a sequence disclosed in that Table (e.g. Table 1-4), which satisfies the requirements of the oligonucleotide probes as described herein, e.g. in length and function. Preferably said parts have the size described hereinafter.

Preferably the oligonucleotide probes forming said set are at least 15 bases in length to allow binding of target molecules. Especially preferably said oligonucleotide probes are from 20 to 200 bases in length, e.g. from 30 to 150 bases, preferably 50-100 bases in length.

As referred to herein the term “complementary sequences” refers to sequences with consecutive complementary bases (ie. T:A, G:C) and which complementary sequences are therefore able to bind to one another through their complementarity.

Reference to “10 oligonucleotides” refers to 10 different oligonucleotides. Whilst a Table 1 oligonucleotide, a Table 1 derived oligonucleotide and their functional equivalent are considered different oligonucleotides, complementary oligonucleotides are not considered different. Preferably however, the at least 10 oligonucleotides are 10 different Table 1 oligonucleotides (or Table 1 derived oligonucleotides or their functional equivalents). Thus said 10 different oligonucleotides are preferably able to bind to 10 different transcripts.

Preferably said oligonucleotides are as described in Table 1 or are derived from a sequence described in Table 1. Especially preferably said oligonucleotides are as described in Table 2 or Table 4 or are derived from a sequence described in either of those tables. Especially preferably the oligonucleotide (or the oligonucleotide derived therefrom) has a high occurrence as defined in Table 3, especially preferably >40%, e.g. >80 or >90, e.g. 100%.

A “set” as described refers to a collection of unique oligonucleotide probes (ie. having a distinct sequence) and preferably consists of less than 1000 oligonucleotide probes, especially less than 500 probes, e.g. preferably from 10 to 500, e.g. 10 to 100, 200 or 300, especially preferably 20 to 100, e.g. 30 to 100 probes. In some cases less than 10 probes may be used, e.g. from 2 to 9 probes, e.g. 5 to 9 probes.

It will be appreciated that increasing the number of probes will prevent the possibility of poor analysis, e.g. misdiagnosis by comparison to other diseases which could similarly alter the expression of the particular genes in question. Other oligonucleotide probes not described herein may also be present, particularly if they aid the ultimate use of the set of oligonucleotide probes. However, preferably said set consists only of said Table 1 oligonucleotides, Table 1 derived oligonucleotides, complementary sequences or functionally equivalent oligonucleotides, or a sub-set thereof (e.g. of the size as described above), preferably a sub-set for which sequences are provided herein (see Table 1 and its footnote). Especially preferably said set consists only of said Table 1 oligonucleotides, Table 1 derived oligonucleotides, or complementary sequences thereof, or a sub-set thereof.

Multiple copies of each unique oligonucleotide probe, e.g. 10 or more copies, may be present in each set, but constitute only a single probe.

A set of oligonucleotide probes, which may preferably be immobilized on a solid support or have means for such immobilization, comprises the at least 10 oligonucleotide probes selected from those described hereinbefore. Especially preferably said probes are selected from those having high occurrence as described in Table 3 and as mentioned above. As mentioned above, these 10 probes must be unique and have different sequences. Having said this however, two separate probes may be used which recognize the same gene but reflect different splicing events. However oligonucleotide probes which are complementary to, and bind to distinct genes are preferred.

As described herein a “functionally equivalent” oligonucleotide to those described in Table 1 or derived therefrom refers to an oligonucleotide which is capable of identifying the same gene as an oligonucleotide of Table 1 or derived therefrom, ie. it can bind to the same mRNA molecule (or DNA) transcribed from a gene (target nucleic acid molecule) as the Table 1 oligonucleotide or the Table 1 derived oligonucleotide (or its complementary sequence). Preferably said functionally equivalent oligonucleotide is capable of recognizing, ie. binding to the same splicing product as a Table 1 oligonucleotide or a Table 1 derived oligonucleotide. Preferably said mRNA molecule is the full length mRNA molecule which corresponds to the Table 1 oligonucleotide or the Table 1 derived oligonucleotide.

As referred to herein “capable of binding” or “binding” refers to the ability to hybridize under conditions described hereinafter.

Alternatively expressed, functionally equivalent oligonucleotides (or complementary sequences) have sequence identity or will hybridize, as described hereinafter, to a region of the target molecule to which molecule a Table 1 oligonucleotide or a Table 1 derived oligonucleotide or a complementary oligonucleotide binds. Preferably, functionally equivalent oligonucleotides (or their complementary sequences) hybridize to one of the mRNA sequences which corresponds to a Table 1 oligonucleotide or a Table 1 derived oligonucleotide under the conditions described hereinafter or has sequence identity to a part of one of the mRNA sequences which corresponds to a Table 1 oligonucleotide or a Table 1 derived oligonucleotide. A “part” in this context refers to a stretch of at least 5, e.g. at least 10 or 20 bases, such as from 5 to 100, e.g. 10 to 50 or 15 to 30 bases.

In a particularly preferred aspect, the functionally equivalent oligonucleotide binds to all or a part of the region of a target nucleic acid molecule (mRNA or cDNA) to which the Table 1 oligonucleotide or Table 1 derived oligonucleotide binds. A “target” nucleic acid molecule is the gene transcript or related product e.g. mRNA, or cDNA, or amplified product thereof. Said “region” of said target molecule to which said Table 1 oligonucleotide or Table 1 derived oligonucleotide binds is the stretch over which complementarity exists. At its largest this region is the whole length of the Table 1 oligonucleotide or Table 1 derived oligonucleotide, but may be shorter if the entire Table 1 sequence or Table 1 derived oligonucleotide is not complementary to a region of the target sequence.

Preferably said part of said region of said target molecule is a stretch of at least 5, e.g. at least 10 or 20 bases, such as from 5 to 100, e.g. 10 to 50 or 15 to 30 bases. This may for example be achieved by said functionally equivalent oligonucleotide having several identical bases to the bases of the Table 1 oligonucleotide or the Table 1 derived oligonucleotide.

These bases may be identical over consecutive stretches, e.g. in a part of the functionally equivalent oligonucleotide, or may be present non-consecutively, but provide sufficient complementarity to allow binding to the target sequence.

Thus in a preferred feature, said functionally equivalent oligonucleotide hybridizes under conditions of high stringency to a Table 1 oligonucleotide or a Table 1 derived oligonucleotide or the complementary sequence thereof. Alternatively expressed, said functionally equivalent oligonucleotide exhibits high sequence identity to all or part of a Table 1 oligonucleotide. Preferably said functionally equivalent oligonucleotide has at least 70% sequence identity, preferably at least 80%, e.g. at least 90, 95, 98 or 99%, to all of a Table 1 oligonucleotide or a part thereof. As used in this context, a “part” refers to a stretch of at least 5, e.g. at least 10 or 20 bases, such as from 5 to 100, e.g. 10 to 50 or 15 to 30 bases, in said Table 1 oligonucleotide. Especially preferably when sequence identity to only a part of said Table 1 oligonucleotide is present, the sequence identity is high, e.g. at least 80% as described above.

Functionally equivalent oligonucleotides which satisfy the above stated functional requirements include those which are derived from the Table 1 oligonucleotides and also those which have been modified by single or multiple nucleotide base (or equivalent) substitution, addition and/or deletion, but which nonetheless retain functional activity, e.g. bind to the same target molecule as the Table 1 oligonucleotide or the Table 1 derived oligonucleotide from which they are further derived or modified. Preferably said modification is of from 1 to 50, e.g. from 10 to 30, preferably from 1 to 5 bases. Especially preferably only minor modifications are present, e.g. variations in less than 10 bases, e.g. less than 5 base changes.

Within the meaning of “addition” equivalents are included oligonucleotides containing additional sequences which are complementary to the consecutive stretch of bases on the target molecule to which the Table 1 oligonucleotide or the Table 1 derived oligonucleotide binds. Alternatively the addition may comprise a different, unrelated sequence, which may for example confer a further property, e.g. to provide a means for immobilization such as a linker to bind the oligonucleotide probe to a solid support.

Particularly preferred are naturally occurring equivalents such as biological variants, e.g. allelic, geographical or allotypic variants, e.g. oligonucleotides which correspond to a genetic variant, for example as present in a different species.

Functional equivalents include oligonucleotides with modified bases, e.g. using non-naturally occurring bases. Such derivatives may be prepared during synthesis or by post production modification.

“Hybridizing” sequences which bind under conditions of low stringency are those which bind under non-stringent conditions (for example, 6×SSC/50% formamide at room temperature) and remain bound when washed under conditions of low stringency (2×SSC, room temperature, more preferably 2×SSC, 42° C.). Hybridizing under high stringency refers to the above conditions in which washing is performed at 2×SSC, 65° C. (where SSC=0.15M NaCl, 0.015M sodium citrate, pH 7.2).

“Sequence identity” as referred to herein refers to the value obtained when assessed using ClustalW (Thompson et al., 1994, Nucl. Acids Res., 22, p 4673-4680) with the following parameters:

Pairwise alignment parameters—Method: accurate,
Matrix: IUB, Gap open penalty: 15.00, Gap extension penalty: 6.66;
Multiple alignment parameters—Matrix: IUB, Gap open penalty: 15.00, % identity for delay: 30, Negative matrix: no, Gap extension penalty: 6.66, DNA transitions weighting: 0.5.

Sequence identity at a particular base is intended to include identical bases which have simply been derivatized.

The invention also extends to polypeptides encoded by the mRNA sequence to which a Table 1 oligonucleotide or a Table 1 derived oligonucleotide binds. The invention further extends to antibodies which bind to any of said polypeptides.

As described above, conveniently said set of oligonucleotide probes may be immobilized on one or more solid supports. Single or preferably multiple copies of each unique probe are attached to said solid supports, e.g. 10 or more, e.g. at least 100 copies of each unique probe are present.

One or more unique oligonucleotide probes may be associated with separate solid supports which together form a set of probes immobilized on multiple solid support, e.g. one or more unique probes may be immobilized on multiple beads, membranes, filters, biochips etc. which together form a set of probes, which together form modules of the kit described hereinafter.

The solid support of the different modules are conveniently physically associated although the signals associated with each probe (generated as described hereinafter) must be separately determinable.

Alternatively, the probes may be immobilized on discrete portions of the same solid support, e.g. each unique oligonucleotide probe, e.g. in multiple copies, may be immobilized to a distinct and discrete portion or region of a single filter or membrane, e.g. to generate an array.

A combination of such techniques may also be used, e.g. several solid supports may be used which each immobilize several unique probes.

The expression “solid support” shall mean any solid material able to bind oligonucleotides by hydrophobic, ionic or covalent bridges.

“Immobilization” as used herein refers to reversible or irreversible association of the probes to said solid support by virtue of such binding. If reversible, the probes remain associated with the solid support for a time sufficient for methods of the invention to be carried out.

Numerous solid supports suitable as immobilizing moieties according to the invention, are well known in the art and widely described in the literature and generally speaking, the solid support may be any of the well-known supports or matrices which are currently widely used or proposed for immobilization, separation etc. in chemical or biochemical procedures. Such materials include, but are not limited to, any synthetic organic polymer such as polystyrene, polyvinylchloride, polyethylene; or nitrocellulose and cellulose acetate; or tosyl activated surfaces; or glass or nylon or any surface carrying a group suited for covalent coupling of nucleic acids. The immobilizing moieties may take the form of particles, sheets, gels, filters, membranes, microfibre strips, tubes or plates, fibres or capillaries, made for example of a polymeric material e.g. agarose, cellulose, alginate, teflon, latex or polystyrene or magnetic beads. Solid supports allowing the presentation of an array, preferably in a single dimension are preferred, e.g. sheets, filters, membranes, plates or biochips.

Attachment of the nucleic acid molecules to the solid support may be performed directly or indirectly. For example if a filter is used, attachment may be performed by UV-induced crosslinking. Alternatively, attachment may be performed indirectly by the use of an attachment moiety carried on the oligonucleotide probes and/or solid support. Thus for example, a pair of affinity binding partners may be used, such as avidin, streptavidin or biotin, DNA or DNA binding protein (e.g. either the lac I repressor protein or the lac operator sequence to which it binds), antibodies (which may be mono- or polyclonal), antibody fragments or the epitopes or haptens of antibodies. In these cases, one partner of the binding pair is attached to (or is inherently part of) the solid support and the other partner is attached to (or is inherently part of) the nucleic acid molecules.

As used herein an “affinity binding pair” refers to two components which recognize and bind to one another specifically (ie. in preference to binding to other molecules). Such binding pairs when bound together form a complex.

Attachment of appropriate functional groups to the solid support may be performed by methods well known in the art, which include for example, attachment through hydroxyl, carboxyl, aldehyde or amino groups which may be provided by treating the solid support to provide suitable surface coatings. Solid supports presenting appropriate moieties for attachment of the binding partner may be produced by routine methods known in the art.

Attachment of appropriate functional groups to the oligonucleotide probes of the invention may be performed by ligation or introduced during synthesis or amplification, for example using primers carrying an appropriate moiety, such as biotin or a particular sequence for capture.

Conveniently, the set of probes described hereinbefore is provided in kit form.

Thus viewed from a further aspect the present invention provides a kit comprising a set of oligonucleotide probes as described hereinbefore immobilized on one or more solid supports.

Preferably, said probes are immobilized on a single solid support and each unique probe is attached to a different region of said solid support. However, when attached to multiple solid supports, said multiple solid supports form the modules which make up the kit. Especially preferably said solid support is a sheet, filter, membrane, plate or biochip.

Optionally the kit may also contain information relating to the signals generated by normal or diseased samples (as discussed in more detail hereinafter in relation to the use of the kits), standardizing materials, e.g. mRNA or cDNA from normal and/or diseased samples for comparative purposes, labels for incorporation into cDNA, adapters for introducing nucleic acid sequences for amplification purposes, primers for amplification and/or appropriate enzymes, buffers and solutions. Optionally said kit may also contain a package insert describing how the method of the invention should be performed, optionally providing standard graphs, data or software for interpretation of results obtained when performing the invention.

The use of such kits to prepare a standard diagnostic gene transcript pattern as described hereinafter forms a further aspect of the invention.

The set of probes as described herein have various uses. Principally however they are used to assess the gene expression state of a test cell to provide information relating to the organism from which said cell is derived. Thus the probes are useful in diagnosing, identifying or monitoring a disease or condition or stage thereof in an organism.

Thus in a further aspect the invention provides the use of a set of oligonucleotide probes or a kit as described hereinbefore to determine the gene expression pattern of a cell which pattern reflects the level of gene expression of genes to which said oligonucleotide probes bind, comprising at least the steps of:

a) isolating mRNA from said cell, which may optionally be reverse transcribed to cDNA;

b) hybridizing the mRNA or cDNA of step (a) to a set of oligonucleotide probes or a kit as defined herein; and

c) assessing the amount of mRNA or cDNA hybridizing to each of said probes to produce said pattern.

The mRNA and cDNA as referred to in this method, and the methods hereinafter, encompass derivatives or copies of said molecules, e.g. copies of such molecules such as those produced by amplification or the preparation of complementary strands, but which retain the identity of the mRNA sequence, ie. would hybridize to the direct transcript (or its complementary sequence) by virtue of precise complementarity, or sequence identity, over at least a region of said molecule. It will be appreciated that complementarity will not exist over the entire region where techniques have been used which may truncate the transcript or introduce new sequences, e.g. by primer amplification. For convenience, said mRNA or cDNA is preferably amplified prior to step b). As with the oligonucleotides described herein said molecules may be modified, e.g. by using non-natural bases during synthesis providing complementarity remains. Such molecules may also carry additional moieties such as signalling or immobilizing means.

The various steps involved in the method of preparing such a pattern are described in more detail hereinafter.

As used herein “gene expression” refers to transcription of a particular gene to produce a specific mRNA product (ie. a particular splicing product). The level of gene expression may be determined by assessing the level of transcribed mRNA molecules or cDNA molecules reverse transcribed from the mRNA molecules or products derived from those molecules, e.g. by amplification.

The “pattern” created by this technique refers to information which, for example, may be represented in tabular or graphical form and conveys information about the signal associated with two or more oligonucleotides.

Preferably said pattern is expressed as an array of numbers relating to the expression level associated with each probe.

Preferably, said pattern is established using the following linear model:

y=Xb+f Equation 1

wherein, X is the matrix of gene expression data and y is the response variable, b is the regression coefficient vector and f the estimated residual vector. Although many different methods can be used to establish the relationship provided in equation 1, especially preferably the partial Least Squares Regression (PLSR) method is used for establishing the relationship in equation 1.

The probes are thus used to generate a pattern which reflects the gene expression of a cell at the time of its isolation. The pattern of expression is characteristic of the circumstances under which that cells finds itself and depends on the influences to which the cell has been exposed. Thus, a characteristic gene transcript pattern standard or fingerprint (standard probe pattern) for cells from an individual with a particular disease or condition may be prepared and used for comparison to transcript patterns of test cells. This has clear applications in diagnosing, monitoring or identifying whether an organism is suffering from a particular disease, condition or stage thereof.

The standard pattern is prepared by determining the extent of binding of total mRNA (or cDNA or related product), from cells from a sample of one or more organisms with the disease or condition or stage thereof, to the probes. This reflects the level of transcripts which are present which correspond to each unique probe. The amount of nucleic acid material which binds to the different probes is assessed and this information together forms the gene transcript pattern standard of that disease or condition or stage thereof.

Each such standard pattern is characteristic of the disease, condition or stage thereof.

In a further aspect therefore, the present invention provides a method of preparing a standard gene transcript pattern characteristic of a disease or condition or stage thereof in an organism comprising at least the steps of:

a) isolating mRNA from the cells of a sample of one or more organisms having the disease or condition or stage thereof, which may optionally be reverse transcribed to cDNA;

b) hybridizing the mRNA or cDNA of step (a) to a set of oligonucleotides or a kit as described hereinbefore specific for said disease or condition or stage thereof in an organism and sample thereof corresponding to the organism and sample thereof under investigation; and

c) assessing the amount of mRNA or cDNA hybridizing to each of said probes to produce a characteristic pattern reflecting the level of gene expression of genes to which said oligonucleotides bind, in the sample with the disease, condition or stage thereof.

For convenience, said oligonucleotides are preferably immobilized on one or more solid supports.

The standard pattern for a great number of diseases or conditions and different stages thereof using particular probes may be accumulated in databases and be made available to laboratories on request.

“Disease” samples and organisms as referred to herein refer to organisms (or samples from the same) with an underlying pathological disturbance relative to a normal organism (or sample), in a symptomatic or asymptomatic organism, which may result, for example, from infection or an acquired or congenital genetic imperfection. Such organisms are known to have, or which exhibit, the disease or condition or stage thereof under study.

A “condition” refers to a state of the mind or body of an organism which has not occurred through disease, e.g. the presence of an agent in the body such as a toxin, drug or pollutant, or pregnancy.

“Stages” thereof refer to different stages of the disease or condition which may or may not exhibit particular physiological or metabolic changes, but do exhibit changes at the genetic level which may be detected as altered gene expression. It will be appreciated that during the course of a disease or condition the expression of different transcripts may vary. Thus at different stages, altered expression may not be exhibited for particular transcripts compared to “normal” samples. However, combining information from several transcripts which exhibit altered expression at one or more stages through the course of the disease or condition can be used to provide a characteristic pattern which is indicative of a particular stage of the disease or condition. Thus for example different stages in cancer, e.g. pre-stage I, stage I, stage II, II or IV can be identified.

“Normal” as used herein refers to organisms or samples which are used for comparative purposes.

Preferably, these are “normal” in the sense that they do not exhibit any indication of, or are not believed to have, any disease or condition that would affect gene expression, particularly in respect of the disease for which they are to be used as the normal standard. However, it will be appreciated that different stages of a disease or condition may be compared and in such cases, the “normal” sample may correspond to the earlier stage of the disease or condition.

As used herein a “sample” refers to any material obtained from the organism, e.g. human or non-human animal under investigation which contains cells and includes, tissues, body fluid or body waste or in the case of prokaryotic organisms, the organism itself. “Body fluids” include blood, saliva, spinal fluid, semen, lymph. “Body waste” includes urine, expectorated matter (pulmonary patients), faeces etc. “Tissue samples” include tissue obtained by biopsy, by surgical interventions or by other means e.g. placenta. Preferably however, the samples which are examined are from areas of the body not apparently affected by the disease or condition. The cells in such samples are not disease cells, e.g. cancer cells, have not been in contact with such disease cells and do not originate from the site of the disease or condition. The “site of disease” is considered to be that area of the body which manifests the disease in a way which may be objectively determined, e.g. a tumour or area of inflammation. Thus for example peripheral blood may be used for the diagnosis of non-haematopoietic cancers, and the blood does not require the presence of malignant or disseminated cells from the cancer in the blood. Similarly in diseases of the brain, in which no diseased cells are found in the blood due to the blood:brain barrier, peripheral blood may still be used in the methods of the invention.

It will however be appreciated that the method of preparing the standard transcription pattern and other methods of the invention are also applicable for use on living parts of eukaryotic organisms such as cell lines and organ cultures and explants. As used herein, reference to “corresponding” sample etc. refers to cells preferably from the same tissue, body fluid or body waste, but also includes cells from tissue, body fluid or body waste which are sufficiently similar for the purposes of preparing the standard or test pattern. When used in reference to genes “corresponding” to the probes, this refers to genes which are related by sequence (which may be complementary) to the probes although the probes may reflect different splicing products of expression.

“Assessing” as used herein refers to both quantitative and qualitative assessment which may be determined in absolute or relative terms.

The invention may be put into practice as follows.

To prepare a standard transcript pattern for a particular disease, condition or stage thereof, sample mRNA is extracted from the cells of tissues, body fluid or body waste according to known techniques (see for example Sambrook et. al. (1989), Molecular Cloning: A laboratory manual, 2nd Ed., Cold Spring Harbor Laboratory Press, Cold Spring Harbor, N.Y.) from a diseased individual or organism.

Owing to the difficulties in working with RNA, the RNA is preferably reverse transcribed at this stage to form first strand cDNA. Cloning of the cDNA or selection from, or using, a cDNA library is not however necessary in this or other methods of the invention. Preferably, the complementary strands of the first strand cDNAs are synthesized, ie. second strand cDNAs, but this will depend on which relative strands are present in the oligonucleotide probes. The RNA may however alternatively be used directly without reverse transcription and may be labelled if so required.

Preferably the cDNA strands are amplified by known amplification techniques such as the polymerase chain reaction (PCR) by the use of appropriate primers. Alternatively, the cDNA strands may be cloned with a vector, used to transform a bacteria such as E. coli which may then be grown to multiply the nucleic acid molecules. When the sequence of the cDNAs are not known, primers may be directed to regions of the nucleic acid molecules which have been introduced. Thus for example, adapters may be ligated to the cDNA molecules and primers directed to these portions for amplification of the cDNA molecules. Alternatively, in the case of eukaryotic samples, advantage may be taken of the polyA tail and cap of the RNA to prepare appropriate primers.

To produce the standard diagnostic gene transcript pattern or fingerprint for a particular disease or condition or stage thereof, the above described oligonucleotide probes are used to probe mRNA or cDNA of the diseased sample to produce a signal for hybridization to each particular oligonucleotide probe species, ie. each unique probe. A standard control gene transcript pattern may also be prepared if desired using mRNA or cDNA from a normal sample. Thus, mRNA or cDNA is brought into contact with the oligonucleotide probe under appropriate conditions to allow hybridization.

When multiple samples are probed, this may be performed consecutively using the same probes, e.g. on one or more solid supports, ie. on probe kit modules, or by simultaneously hybridizing to corresponding probes, e.g. the modules of a corresponding probe kit.

To identify when hybridization occurs and obtain an indication of the number of transcripts/cDNA molecules which become bound to the oligonucleotide probes, it is necessary to identify a signal produced when the transcripts (or related molecules) hybridize (e.g. by detection of double stranded nucleic acid molecules or detection of the number of molecules which become bound, after removing unbound molecules, e.g. by washing).

In order to achieve a signal, either or both components which hybridize (ie. the probe and the transcript) carry or form a signalling means or a part thereof. This “signalling means” is any moiety capable of direct or indirect detection by the generation or presence of a signal. The signal may be any detectable physical characteristic such as conferred by radiation emission, scattering or absorption properties, magnetic properties, or other physical properties such as charge, size or binding properties of existing molecules (e.g. labels) or molecules which may be generated (e.g. gas emission etc.). Techniques are preferred which allow signal amplification, e.g. which produce multiple signal events from a single active binding site, e.g. by the catalytic action of enzymes to produce multiple detectable products.

Conveniently the signalling means may be a label which itself provides a detectable signal. Conveniently this may be achieved by the use of a radioactive or other label which may be incorporated during cDNA production, the preparation of complementary cDNA strands, during amplification of the target mRNA/cDNA or added directly to target nucleic acid molecules.

Appropriate labels are those which directly or indirectly allow detection or measurement of the presence of the transcripts/cDNA. Such labels include for example radiolabels, chemical labels, for example chromophores or fluorophores (e.g. dyes such as fluorescein and rhodamine), or reagents of high electron density such as ferritin, haemocyanin or colloidal gold.

Alternatively, the label may be an enzyme, for example peroxidase or alkaline phosphatase, wherein the presence of the enzyme is visualized by its interaction with a suitable entity, for example a substrate. The label may also form part of a signalling pair wherein the other member of the pair is found on, or in close proximity to, the oligonucleotide probe to which the transcript/cDNA binds, for example, a fluorescent compound and a quench fluorescent substrate may be used.

A label may also be provided on a different entity, such as an antibody, which recognizes a peptide moiety attached to the transcripts/cDNA, for example attached to a base used during synthesis or amplification.

A signal may be achieved by the introduction of a label before, during or after the hybridization step. Alternatively, the presence of hybridizing transcripts may be identified by other physical properties, such as their absorbance, and in which case the signalling means is the complex itself.

The amount of signal associated with each oligonucleotide probe is then assessed. The assessment may be quantitative or qualitative and may be based on binding of a single transcript species (or related cDNA or other products) to each probe, or binding of multiple transcript species to multiple copies of each unique probe. It will be appreciated that quantitative results will provide further information for the transcript fingerprint of the disease which is compiled. This data may be expressed as absolute values (in the case of macroarrays) or may be determined relative to a particular standard or reference e.g. a normal control sample.

Furthermore it will be appreciated that the standard diagnostic gene pattern transcript may be prepared using one or more disease samples (and normal samples if used) to perform the hybridization step to obtain patterns not biased towards a particular individual's variations in gene expression.

The use of the probes to prepare standard patterns and the standard diagnostic gene transcript patterns thus produced for the purpose of identification or diagnosis or monitoring of a particular disease or condition or stage thereof in a particular organism forms a further aspect of the invention.

Once a standard diagnostic fingerprint or pattern has been determined for a particular disease or condition using the selected oligonucleotide probes, this information can be used to identify the presence, absence or extent or stage of that disease or condition in a different test organism or individual.

To examine the gene expression pattern of a test sample, a test sample of tissue, body fluid or body waste containing cells, corresponding to the sample used for the preparation of the standard pattern, is obtained from a patient or the organism to be studied. A test gene transcript pattern is then prepared as described hereinbefore as for the standard pattern.

In a further aspect therefore, the present invention provides a method of preparing a test gene transcript pattern comprising at least the steps of:

a) isolating mRNA from the cells of a sample of said test organism, which may optionally be reverse transcribed to cDNA;

b) hybridizing the mRNA or cDNA of step (a) to a set of oligonucleotides or a kit as described hereinbefore specific for a disease or condition or stage thereof in an organism and sample thereof corresponding to the organism and sample thereof under investigation; and

c) assessing the amount of mRNA or cDNA hybridizing to each of said probes to produce said pattern reflecting the level of gene expression of genes to which said oligonucleotides bind, in said test sample.

This test pattern may then be compared to one or more standard patterns to assess whether the sample contains cells having the disease, condition or stage thereof.

Thus viewed from a further aspect the present invention provides a method of diagnosing or identifying or monitoring a disease or condition or stage thereof in an organism, comprising the steps of:
a) isolating mRNA from the cells of a sample of said organism, which may optionally be reverse transcribed to cDNA;
b) hybridizing the mRNA or cDNA of step (a) to a set of oligonucleotides or a kit as described hereinbefore specific for said disease or condition or stage thereof in an organism and sample thereof corresponding to the organism and sample thereof under investigation;
c) assessing the amount of mRNA or cDNA hybridizing to each of said probes to produce a characteristic pattern reflecting the level of gene expression of genes to which said oligonucleotides bind, in said sample; and
d) comparing said pattern to a standard diagnostic pattern prepared according to the method of the invention using a sample from an organism corresponding to the organism and sample under investigation to determine the presence of said disease or condition or a stage thereof in the organism under investigation.

The method up to and including step c) is the preparation of a test pattern as described above.

As referred to herein, “diagnosis” refers to determination of the presence or existence of a disease or condition or stage thereof in an organism. “Monitoring” refers to establishing the extent of a disease or condition, particularly when an individual is known to be suffering from a disease or condition, for example to monitor the effects of treatment or the development of a disease or condition, e.g. to determine the suitability of a treatment or provide a prognosis.

The presence of the disease or condition or stage thereof may be determined by determining the degree of correlation between the standard and test samples' patterns. This necessarily takes into account the range of values which are obtained for normal and diseased samples. Although this can be established by obtaining standard deviations for several representative samples binding to the probes to develop the standard, it will be appreciated that single samples may be sufficient to generate the standard pattern to identify a disease if the test sample exhibits close enough correlation to that standard. Conveniently, the presence, absence, or extent of a disease or condition or stage thereof in a test sample can be predicted by inserting the data relating to the expression level of informative probes in test sample into the standard diagnostic probe pattern established according to equation 1.

Data generated using the above mentioned methods may be analysed using various techniques from the most basic visual representation (e.g. relating to intensity) to more complex data manipulation to identify underlying patterns which reflect the interrelationship of the level of expression of each gene to which the various probes bind, which may be quantified and expressed mathematically. Conveniently, the raw data thus generated may be manipulated by the data processing and statistical methods described hereinafter, particularly normalizing and standardizing the data and fitting the data to a classification model to determine whether said test data reflects the pattern of a particular disease, condition or stage thereof.

The methods described herein may be used to identify, monitor or diagnose a disease, condition or ailment or its stage or progression, for which the oligonucleotide probes are informative. “Informative” probes as described herein, are those which reflect genes which have altered expression in the diseases or conditions in question, or particular stages thereof. Probes of the invention may not be sufficiently informative for diagnostic purposes when used alone, but are informative when used as one of several probes to provide a characteristic pattern, e.g. in a set as described hereinbefore.

Preferably said probes correspond to genes which are systemically affected by said disease, condition or stage thereof. Especially preferably said genes, from which transcripts are derived which bind to probes of the invention, are metabolic or house-keeping genes and preferably are moderately or highly expressed. The advantage of using probes directed to moderately or highly expressed genes is that smaller clinical samples are required for generating the necessary gene expression data set, e.g. less than 1 ml blood samples.

Furthermore, it has been found that such genes which are already being actively transcribed tend to be more prone to being influenced, in a positive or negative way, by new stimuli. In addition, since transcripts are already being produced at levels which are generally detectable, small changes in those levels are readily detectable as for example, a certain detectable threshold does not need to be reached.

In preferred methods of the invention, the set of probes of the invention are informative for a variety of different diseases, conditions or stages thereof. A sub-set of the probes disclosed herein may be used for diagnosis, identification or monitoring a particular disease, condition or stage thereof. Thus the probes may be used to diagnose or identify or monitor any condition, ailment, disease or reaction that leads to the relative increase or decrease in the activity of informative genes of any or all eukaryotic or prokaryotic organisms regardless of whether these changes have been caused by the influence of bacteria, virus, prions, parasites, fungi, radiation, natural or artificial toxins, drugs or allergens, including mental conditions due to stress, neurosis, psychosis or deteriorations due to the ageing of the organism, and conditions or diseases of unknown cause, providing a sub-set of the probes as described herein are informative for said disease or condition or stage thereof.

Such diseases include those which result in metabolic or physiological changes, such as fever-associated diseases such as influenza or malaria. Other diseases which may be detected include for example yellow fever, sexually transmitted diseases such as gonorrhea, fibromyalgia, candida-related complex, cancer (for example of the stomach, lung, breast, prostate gland, bowel, skin, colon, ovary etc), Alzheimer's disease, disease caused by retroviruses such as HIV, senile dementia, multiple sclerosis and Creutzfeldt-Jakob disease to mention a few.

The invention may also be used to identify patients with psychiatric or psychosomatic diseases such as schizophrenia and eating disorders. Of particular importance is the use of this method to detect diseases, conditions, or stages thereof, which are not readily detectable by known diagnostic methods, such as HIV which is generally not detectable using known techniques 1 to 4 months following infection. Conditions which may be identified include for example drug abuse, such as the use of narcotics, alcohol, steroids or performance enhancing drugs.

Preferably said disease to be identified or monitored is a cancer or a degenerative brain disorder (such as Alzheimer's or Parkinson's disease).

In particular, a set of oligonucleotide probes,

wherein said set comprises at least 10 oligonucleotides selected from:
an oligonucleotide as described in Table 4 or an oligonucleotide derived therefrom or an oligonucleotide with a complementary sequence, or a functionally equivalent oligonucleotide,
   may be used for diagnosis or identification or monitoring the progression of Alzheimer's disease. Similarly Table 2 probes and Table 2 derived probes and their functional equivalents may be used to diagnose, identify or monitor the progression of breast cancer. Especially preferably the probes used for breast cancer analysis are selected based on their occurrence as set forth in Table 3 and as described hereinbefore.

The diagnostic method may be used alone as an alternative to other diagnostic techniques or in addition to such techniques. For example, methods of the invention may be used as an alternative or additive diagnostic measure to diagnosis using imaging techniques such as Magnetic Resonance Imagine (MRI), ultrasound imaging, nuclear imaging or X-ray imaging, for example in the identification and/or diagnosis of tumours.

The methods of the invention may be performed on cells from prokaryotic or eukaryotic organisms which may be any eukaryotic organisms such as human beings, other mammals and animals, birds, insects, fish and plants, and any prokaryotic organism such as a bacteria.

Preferred non-human animals on which the methods of the invention may be conducted include, but are not limited to mammals, particularly primates, domestic animals, livestock and laboratory animals. Thus preferred animals for diagnosis include mice, rats, guinea pigs, cats, dogs, pigs, cows, goats, sheep, horses. Particularly preferably the disease state or condition of humans is diagnosed, identified or monitored.

As described above, the sample under study may be any convenient sample which may be obtained from an organism. Preferably however, as mentioned above, the sample is obtained from a site distant to the site of disease and the cells in such samples are not disease cells, have not been in contact with such cells and do not originate from the site of the disease or condition.

In such cases, although preferably absent, the sample may contain cells which do not fulfil these criteria. However, since the probes of the invention are concerned with transcripts whose expression is altered in cells which do satisfy these criteria, the probes are specifically directed to detecting changes in transcript levels in those cells even if in the presence of other, background cells.

It has been found that the cells from such samples show significant and informative variations in the gene expression of a large number of genes. Thus, the same probe (or several probes) may be found to be informative in determinations regarding two or more diseases, conditions or stages thereof by virtue of the particular level of transcripts binding to that probe or the interrelationship of the extent of binding to that probe relative to other probes. As a consequence, it is possible to use a relatively small number of probes for screening for multiple disorders or diseases. This has consequences with regard to the selection of probes, discussed in relation to random identification of probes hereinafter, but also for the use of a single set of probes for more than one diagnosis. Table 9 which represents preferred probes of the invention discloses probes which are informative for both Alzheimer's and breast cancer.

Thus, the present invention also provides sets of probes for diagnosing, identifying or monitoring two or more diseases, conditions or stages thereof, wherein at least one of said probes is suitable for said diagnosing, identifying or monitoring at least two of said diseases, conditions or stages thereof, and kits and methods of using the same. Preferably at least 5 probes, e.g. from 5 to 15 probes, are used in at least two diagnoses.

Thus, in a further preferred aspect, the present invention provides a method of diagnosis or identification or monitoring as described hereinbefore for the diagnosis, identification or monitoring of two or more diseases, conditions or stages thereof in an organism, wherein said test pattern produced in step c) of the diagnostic method is compared in step d) to at least two standard diagnostic patterns prepared as described previously, wherein each standard diagnostic pattern is a pattern generated for a different disease or condition or stage thereof.

Whilst in a preferred aspect the methods of assessment concern the development of a gene transcript pattern from a test sample and comparison of the same to a standard pattern, the elevation or depression of expression of certain markers may also be examined by examining the products of expression and the level of those products. Thus a standard pattern in relation to the expressed product may be generated.

In such methods the levels of expression of a set of polypeptides encoded by the gene to which an oligonucleotide of Table 1 or a Table 1 derived oligonucleotide, binds, are analysed.

Various diagnostic methods may be used to assess the amount of polypeptides (or fragments thereof) which are present. The presence or concentration of polypeptides may be examined, for example by the use of a binding partner to said polypeptide (e.g. an antibody), which may be immobilized, to separate said polypeptide from the sample and the amount of polypeptide may then be determined.

“Fragments” of the polypeptides refers to a domain or region of said polypeptide, e.g. an antigenic fragment, which is recognizable as being derived from said polypeptide to allow binding of a specific binding partner. Preferably such a fragment comprises a significant portion of said polypeptide and corresponds to a product of normal post-synthesis processing. Thus in a further aspect the present invention provides a method of preparing a standard gene transcript pattern characteristic of a disease or condition or stage thereof in an organism comprising at least the steps of:

a) releasing target polypeptides from a sample of one or more organisms having the disease or condition or stage thereof;

b) contacting said target polypeptides with one or more binding partners, wherein each binding partner is specific to a marker polypeptide (or a fragment thereof) encoded by the gene to which an oligonucleotide of Table 1 (or derived from a sequence described in Table 1) binds, to allow binding of said binding partners to said target polypeptides, wherein said marker polypeptides are specific for said disease or condition thereof in an organism and sample thereof corresponding to the organism and sample thereof under investigation; and

c) assessing the target polypeptide binding to said binding partners to produce a characteristic pattern reflecting the level of gene expression of genes which express said marker polypeptides, in the sample with the disease, condition or stage thereof.

As used herein “target polypeptides” refer to those polypeptides present in a sample which are to be detected and “marker polypeptides” are polypeptides which are encoded by the genes to which Table 1 oligonucleotides or Table 1 derived oligonucleotides bind. The target and marker polypeptides are identical or at least have areas of high similarity, e.g. epitopic regions to allow recognition and binding of the binding partner.

“Release” of the target polypeptides refers to appropriate treatment of a sample to provide the polypeptides in a form accessible for binding of the binding partners, e.g. by lysis of cells where these are present. The samples used in this case need not necessarily comprise cells as the target polypeptides may be released from cells into the surrounding tissue or fluid, and this tissue or fluid may be analysed, e.g. urine or blood. Preferably however the preferred samples as described herein are used. “Binding partners” comprise the separate entities which together make an affinity binding pair as described above, wherein one partner of the binding pair is the target or marker polypeptide and the other partner binds specifically to that polypeptide, e.g. an antibody.

Various arrangements may be envisaged for detecting the amount of binding pairs which form. In its simplest form, a sandwich type assay e.g. an immunoassay such as an ELISA, may be used in which an antibody specific to the polypeptide and carrying a label (as described elsewhere herein) may be bound to the binding pair (e.g. the first antibody:polypeptide pair) and the amount of label detected.

Other methods as described herein may be similarly modified for analysis of the protein product of expression rather than the gene transcript and related nucleic acid molecules.

Thus a further aspect of the invention provides a method of preparing a test gene transcript pattern comprising at least the steps of:

a) releasing target polypeptides from a sample of said test organism;

b) contacting said target polypeptides with one or more binding partners, wherein each binding partner is specific to a marker polypeptide (or a fragment thereof) encoded by the gene to which an oligonucleotide of Table 1 (or derived from a sequence described in Table 1) binds, to allow binding of said binding partners to said target polypeptides, wherein said marker polypeptides are specific for said disease or condition thereof in an organism and sample thereof corresponding to the organism and sample thereof under investigation; and

c) assessing the target polypeptide binding to said binding partners to produce a characteristic pattern reflecting the level of gene expression of genes which express said marker polypeptides, in said test sample.

A yet further aspect of the invention provides a method of diagnosing or identifying or monitoring a disease or condition or stage thereof in an organism comprising the steps of:

a) releasing target polypeptides from a sample of said organism;

b) contacting said target polypeptides with one or more binding partners, wherein each binding partner is specific to a marker polypeptide (or a fragment thereof) encoded by the gene to which an oligonucleotide of Table 1 (or derived from a sequence described in Table 1) binds, to allow binding of said binding partners to said target polypeptides, wherein said marker polypeptides are specific for said disease or condition thereof in an organism and sample thereof corresponding to the organism and sample thereof under investigation; and

c) assessing the target polypeptide binding to said binding partners to produce a characteristic pattern reflecting the level of gene expression of genes which express said marker polypeptides in said sample; and

d) comparing said pattern to a standard diagnostic pattern prepared as described hereinbefore using a sample from an organism corresponding to the organism and sample under investigation to determine the degree of correlation indicative of the presence of said disease or condition or a stage thereof in the organism under investigation.

The methods of generating standard and test patterns and diagnostic techniques rely on the use of informative oligonucleotide probes to generate the gene expression data. In some cases it will be necessary to select these informative probes for a particular method, e.g. to diagnose a particular disease, from a selection of available probes, e.g. the probes described hereinbefore (the Table 1 oligonucleotides, the Table 1 derived oligonucleotides, their complementary sequences and functionally equivalent oligonucleotides). The following methodology describes a convenient method for identifying such informative probes, or more particularly how to select a suitable sub-set of probes from the probes described herein.

Probes for the analysis of a particular disease or condition or stage thereof, may be identified in a number of ways known in the prior art, including by differential expression or by library subtraction (see for example WO98/49342). As described hereinafter, in view of the high information content of most transcripts, as a starting point one may also simply analyse a random sub-set of mRNA or cDNA species and pick the most informative probes from that sub-set. The following method describes the use of immobilized oligonucleotide probes (e.g. the probes of the invention) to which mRNA (or related molecules) from different samples is bound to identify which probes are the most informative to identify a particular type of sample, e.g. a disease sample.

The immobilized probes can be derived from various unrelated or related organisms; the only requirement is that the immobilized probes should bind specifically to their homologous counterparts in test organisms. Probes can also be derived from commercially available or public databases and immobilized on solid supports or, as mentioned above, they can be randomly picked and isolated from a cDNA library and immobilized on a solid support.

The length of the probes immobilised on the solid support should be long enough to allow for specific binding to the target sequences. The immobilised probes can be in the form of DNA, RNA or their modified products or PNAs (peptide nucleic acids). Preferably, the probes immobilised should bind specifically to their homologous counterparts representing highly and moderately expressed genes in test organisms. Conveniently the probes which are used are the probes described herein.

The gene expression pattern of cells in biological samples can be generated using prior art techniques such as microarray or macroarray as described below or using methods described herein. Several technologies have now been developed for monitoring the expression level of a large number of genes simultaneously in biological samples, such as, high-density oligoarrays (Lockhart et al., 1996, Nat. Biotech., 14, p 1675-1680), cDNA microarrays (Schena et al, 1995, Science, 270, p 467-470) and cDNA macroarrays (Maier E et al., 1994, Nucl. Acids Res., 22, p 3423-3424; Bernard et al., 1996, Nucl. Acids Res., 24, p 1435-1442).

In high-density oligoarrays and cDNA microarrays, hundreds and thousands of probe oligonucleotides or cDNAs, are spotted onto glass slides or nylon membranes, or synthesized on biochips. The mRNA isolated from the test and reference samples are labelled by reverse transcription with a red or green fluorescent dye, mixed, and hybridised to the microarray. After washing, the bound fluorescent dyes are detected by a laser, producing two images, one for each dye. The resulting ratio of the red and green spots on the two images provides the information about the changes in expression levels of genes in the test and reference samples. Alternatively, single channel or multiple channel microarray studies can also be performed.

In cDNA macroarray, different cDNAs are spotted on a solid support such as nylon membranes in excess in relation to the amount of test mRNA that can hybridise to each spot. mRNA isolated from test samples is radio-labelled by reverse transcription and hybridised to the immobilised probe cDNA. After washing, the signals associated with labels hybridising specifically to immobilised probe cDNA are detected and quantified. The data obtained in macroarray contains information about the relative levels of transcripts present in the test samples. Whilst macroarrays are only suitable to monitor the expression of a limited number of genes, microarrays can be used to monitor the expression of several thousand genes simultaneously and is, therefore, a preferred choice for large-scale gene expression studies.

A macroarray technique for generating the gene expression data set has been used to illustrate the probe identification method described herein. For this purpose, mRNA is isolated from samples of interest and used to prepare labelled target molecules, e.g. mRNA or cDNA as described above. The labelled target molecules are then hybridised to probes immobilised on the solid support. Various solid supports can be used for the purpose, as described previously. Following hybridization, unbound target molecules are removed and signals from target molecules hybridizing to immobilised probes quantified. If radio labelling is performed, PhosphoImager can be used to generate an image file that can be used to generate a raw data set. Depending on the nature of label chosen for labelling the target molecules, other instruments can also be used, for example, when fluorescence is used for labelling, a FluoroImager can be used to generate an image file from the hybridised target molecules.

The raw data corresponding to mean intensity, median intensity, or volume of the signals in each spot can be acquired from the image file using commercially available software for image analysis. However, the acquired data needs to be corrected for background signals and normalized prior to analysis, since, several factors can affect the quality and quantity of the hybridising signals. For example, variations in the quality and quantity of mRNA isolated from sample to sample, subtle variations in the efficiency of labelling target molecules during each reaction, and variations in the amount of unspecific binding between different macroarrays can all contribute to noise in the acquired data set that must be corrected for prior to analysis.

Background correction can be performed in several ways. The lowest pixel intensity within a spot can be used for background subtraction or the mean or median of the line of pixels around the spots' outline can be used for the purpose. One can also define an area representing the background intensity based on the signals generated from negative controls and use the average intensity of this area for background subtraction.

The background corrected data can then be transformed for stabilizing the variance in the data structure and normalized for the differences in probe intensity. Several transformation techniques have been described in the literature and a brief overview can be found in Cui, Kerr and Churchill www.jax.org/research/churchill/research/expression/Cui-Transform.pdf). Normalization can be performed by dividing the intensity of each spot with the collective intensity, average intensity or median intensity of all the spots in a macroarray or a group of spots in a macroarray in order to obtain the relative intensity of signals hybridising to immobilised probes in a macroarray. Several methods have been described for normalizing gene expression data (Richmond and Somerville, 2000, Current Opin. Plant Biol., 3, p 108-116; Finkelstein et al., 2001, In “Methods of Microarray Data Analysis. Papers from CAMDA, Eds. Lin & Johnsom, Kluwer Academic, p 57-68; Yang et al., 2001, In “Optical Technologies and Informatics”, Eds. Bittner, Chen, Dorsel & Dougherty, Proceedings of SPIE, 4266, p 141-152; Dudoit et al, 2000, J. Am. Stat. Ass., 97, p 77-87; Alter et al 2000, supra; Newton et al., 2001, J. Comp. Biol., 8, p 37-52). Generally, a scaling factor or function is first calculated to correct the intensity effect and then used for normalising the intensities. The use of external controls has also been suggested for improved normalization.

One other major challenge encountered in large-scale gene expression analysis is that of standardization of data collected from experiments performed at different times. We have observed that gene expression data for samples acquired in the same experiment can be efficiently compared following background correction and normalization. However, the data from samples acquired in experiments performed at different times requires further standardization prior to analysis. This is because subtle differences in experimental parameters between different experiments, for example, differences in the quality and quantity of mRNA extracted at different times, differences in time used for target molecule labelling, hybridization time or exposure time, can affect the measured values. Also, factors such as the nature of the sequence of transcripts under investigation (their GC content) and their amount in relation to the each other determines how they are affected by subtle variations in the experimental processes. They determine, for example, how efficiently first strand cDNAs, corresponding to a particular transcript, are transcribed and labelled during first strand synthesis, or how efficiently the corresponding labelled target molecules bind to their complementary sequences during hybridization. Batch to batch difference in the printing process is also a major factor for variation in the generated expression data.

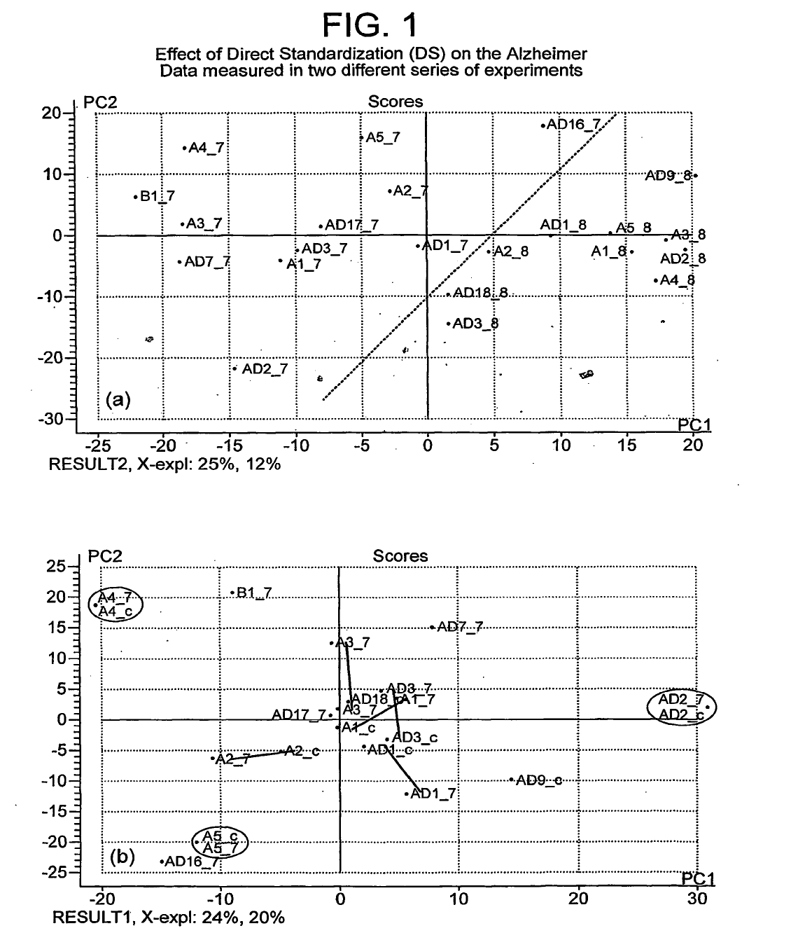

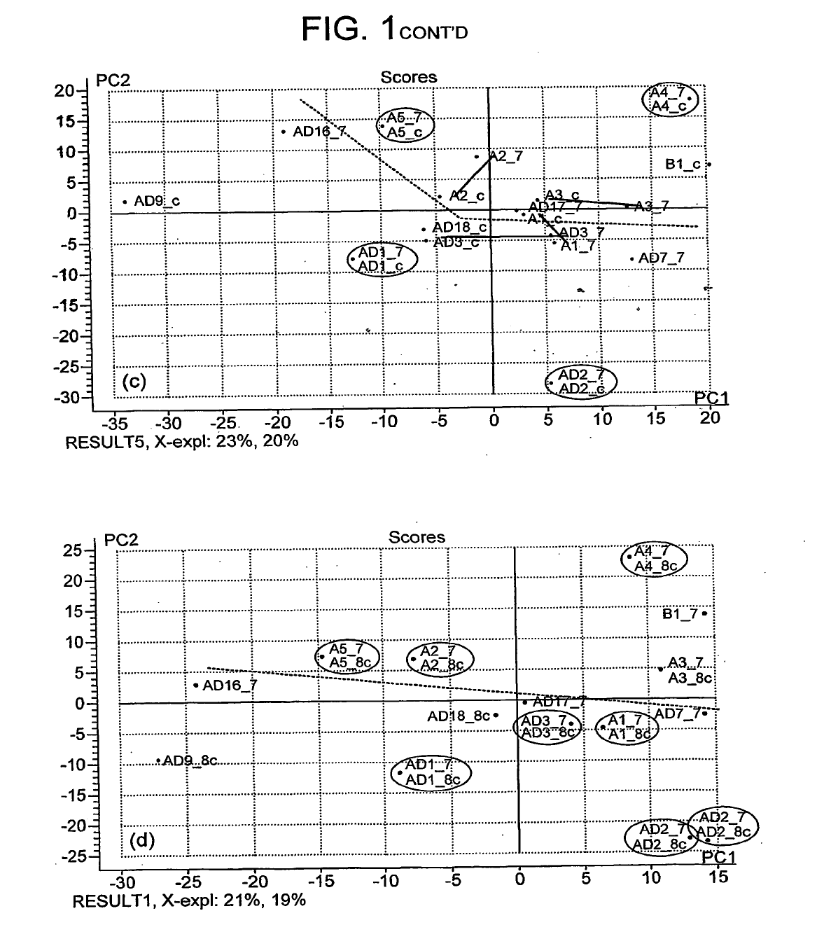

Failure to properly address and rectify for these influences leads to situations where the differences between the experimental series may overshadow the main information of interest contained in the gene expression data set, i.e. the differences within the combined data from the different experimental series. FIG. 1 provides one such example showing a classification based on Principal Component Analysis (PCA) of combined data from two experimental series where the main goal is to distinguish between Alzheimer/non-Alzheimer patients.

PCA (also known as singular value decomposition) is a technique for studying interdependencies and underlying relationships of a set of variables. The data are modelled in terms of a few significant factors or principal components (PC's), plus residuals. The PC's contain the main phenomena and define the systematic variability present in the data, while the residuals represent the variability interpreted as noise. Details on PCA can be found in Jollife (1986, Principal Component Analysis, Springer-Verlag, NY), and Jackson (1991, A User's Guide to Principal Components, Wiley, NY). The results of FIG. 1 show that two clusters are formed representing the data from two experimental series rather than the Alzheimer/non-Alzheimer differentiation. There were eight samples in common between the two series of experiments, which ideally should have fallen on top of, or in near proximity to, each other if appropriately standardized.

We have now found that gene expression data between different experiments can be efficiently standardized by including a subset of samples from one experimental series in the next experimental series and using a direct standardization method (DS), originally described by Wang and Kowalski (Anal. Chem., 1991, 63, p 2750 and J. Chemometrics, 1991, 5, p 129-145). Although the method of DS is well known in the field of analytical chemistry, it remains undescribed and unused in the field of gene expression data analysis.

In DS, the secondary data representing for example experimental series 2 (secondary measurements, R₂) are corrected to match the data measured on the primary measurements representing data from series 1 (R₁), while the calibration model remains unchanged. In DS, response matrices for both experimental series are related to each other by a transformation matrix F, i.e.

R₁=R₂F (1)

Where F is a square matrix dimensioned gene by gene. From (1), the transformation matrix is calculated as:

F=R₂⁺R₁ (2)

The transformation matrix F in equation (2) is calculated using a relatively small subset of samples which are measured on both the master primary and the secondary series of data.

Finally, the response of the unknown sample measured on the secondary series r^{T}_{2,un}, is standardized to the response vector \hat{r}^{T}_{1,un} expected from the primary series

{circumflex over (\hat{r}^{T}_{1,un}=rT_{21,un} [symbol] (3)

From the preceding equation it can be seen that the column i of the transformation matrix contains the multiplication factors for a set of genes measured in the secondary series to obtain the intensity at spot i of the corrected series.

The number of samples that are repeated in the experimental series, R₁ and R₂, should be equal to their ranks, which in this case is equal to the number of principal components retained for explaining the variation in the R₁ and R₂. For example, if three principal components are retained for explaining the variation in the data set, a minimum of three samples should be repeated between R₁ and R₂. The samples that should be repeated between different series should ideally be those that exhibit high leverages in the gene expression pattern. At times, two samples may suffice, while at other times, more than two samples should be ideally be included for good representativity. In some cases, the samples selected can be the same in all the experimental series to be compared (reference samples), while in other cases, representative samples can be selected sequentially by analyzing the expression pattern after each experiment. The selected samples with high leverages are then included in the next experimental series. The results of using Direct Standardization are shown in FIG. 1.

Another approach for normalizing and standardizing the gene expression data set is to hybridize each DNA array with target molecules prepared from a test sample and an equal amount of labelled target molecules prepared from representative reference samples. In order to measure the intensity of labelled target molecules hybridizing to the immobilized probes it is necessary that the labelled molecules are prepared from test and reference samples using different labels, for example, different fluorescent dyes can be used for preparing the labelled material. The labelled molecules prepared from reference samples can be added to the hybridization solution together with the labelled material prepared from test samples. A data file from each array representing the expression pattern of different genes in the test sample and reference samples can then be obtained, normalized and standardized by the direct standardization method as described above. An instant advantage of including the differentially labelled target molecules from reference samples during hybridization is that it enables an efficient comparison of new test samples to the data sets already stored in a database.

Monitoring the expression of a large number of genes in several samples leads to the generation of a large amount of data that is too complex to be easily interpreted. Several unsupervised and supervised multivariate data analysis techniques have already been shown to be useful in extracting meaningful biological information from these large data sets. Cluster analysis is by far the most commonly used technique for gene expression analysis, and has been performed to identify genes that are regulated in a similar manner, and or identifying new/unknown tumour classes using gene expression profiles (Eisen et al., 1998, PNAS, 95, p 14863-14868, Alizadeh et al. 2000, supra, Perou et al. 2000, Nature, 406, p 747-752; Ross et al, 2000, Nature Genetics, 24(3), p 227-235; Herwig et al., 1999, Genome Res., 9, p 1093-1105; Tamayo et al, 1999, Science, PNAS, 96, p 2907-2912).

In the clustering method, genes are grouped into functional categories (clusters) based on their expression profile, satisfying two criteria: homogeneity—the genes in the same cluster are highly similar in expression to each other; and separation—genes in different clusters have low similarity in expression to each other.

Examples of various clustering techniques that have been used for gene expression analysis include hierarchical clustering (Eisen et al., 1998, supra; Alizadeh et al. 2000, supra; Perou et al. 2000, supra; Ross et al, 2000, supra), K-means clustering (Herwig et al., 1999, supra; Tavazoie et al, 1999, Nature Genetics, 22(3), p. 281-285), gene shaving (Hastie et al., 2000, Genome Biology, 1(2), research 0003.1-0003.21), block clustering (Tibshirani et al., 1999, Tech repot Univ Stanford.) Plaid model (Lazzeroni, 2002, Stat. Sinica, 12, p 61-86), and self-organizing maps (Tamayo et al. 1999, supra). Also, related methods of multivariate statistical analysis, such as those using the singular value decomposition (Alter et al., 2000, PNAS, 97(18), p 10101-10106; Ross et al. 2000, supra) or multidimensional scaling can be effective at reducing the dimensions of the objects under study.

However, methods such as cluster analysis and singular value decomposition are purely exploratory and only provide a broad overview of the internal structure present in the data. They are unsupervised approaches in which the available information concerning the nature of the class under investigation is not used in the analysis. Often, the nature of the biological perturbation to which a particular sample has been subjected is known. For example, it is sometimes known whether the sample whose gene expression pattern is being analysed derives from a diseased or healthy individual. In such instances, discriminant analysis can be used for classifying samples into various groups based on their gene expression data.

In such an analysis one builds the classifier by training the data that is capable of discriminating between member and non-members of a given class. The trained classifier can then be used to predict the class of unknown samples. Examples of discrimination methods that have been described in the literature include Support Vector Machines (Brown et al, 2000, PNAS, 97, p 262-267), Nearest Neighbour (Dudoit et al., 2000, supra), Classification trees (Dudoit et al., 2000, supra), Voted classification (Dudoit et al., 2000, supra), Weighted Gene voting (Golub et al. 1999, supra), and Bayesian classification (Keller et al. 2000, Tec report Univ of Washington). Also a technique in which PLS (Partial Least Square) regression analysis is first used to reduce the dimensions in the gene expression data set followed by classification using logistic discriminant analysis and quadratic discriminant analysis (LD and QDA) has recently been described (Nguyen & Rocke, 2002, Bioinformatics, 18, p 39-50 and 1216-1226).

A challenge that gene expression data poses to classical discriminatory methods is that the number of genes whose expression are being analysed is very large compared to the number of samples being analysed.

However in most cases only a small fraction of these genes are informative in discriminant analysis problems. Moreover, there is a danger that the noise from irrelevant genes can mask or distort the information from the informative genes. Several methods have been suggested in literature to identify and select genes that are informative in microarray studies, for example, t-statistics (Dudoit et al, 2002, J. Am. Stat. Ass., 97, p 77-87), analysis of variance (Kerr et al., 2000, PNAS, 98, p 8961-8965), Neighbourhood analysis (Golub et al, 1999, supra), Ratio of between groups to within groups sum of squares (Dudoit et al., 2002, supra), Non parametric scoring (Park et al., 2002, Pacific Symposium on Biocomputing, p 52-63) and Likelihood selection (Keller et al., 2000, supra).

In the methods described herein the gene expression data that has been normalized and standardized is analysed by using Partial Least Squares Regression (PLSR). Although PLSR is primarily a method used for regression analysis of continuous data (see Appendix A), it can also be utilized as a method for model building and discriminant analysis using a dummy response matrix based on a binary coding. The class assignment is based on a simple dichotomous distinction such as breast cancer (class 1)/healthy (class 2), or a multiple distinction based on multiple disease diagnosis such as breast cancer (class 1)/Alzheimer (class 2)/healthy (class 3). The list of diseases for classification can be increased depending upon the samples available corresponding to other diseases or conditions or stages thereof.

PLSR applied as a classification method is referred to as PLS-DA (DA standing for Discriminant analysis). PLS-DA is an extension of the PLSR algorithm in which the Y-matrix is a dummy matrix containing n rows (corresponding to the number of samples) and K columns (corresponding to the number of classes). The Y-matrix is constructed by inserting 1 in the kth column and −1 in all the other columns if the corresponding ith object of X belongs to class k. By regressing Y onto X, classification of a new sample is achieved by selecting the group corresponding to the largest component of the fitted, _(x)=(_{—1}(x), _{—2}(x), . . . , _{—k}(x)). Thus, in a −1/1 response matrix, a prediction value below 0 means that the sample belongs to the class designated as −1, while a prediction value above 0 implies that the sample belongs to the class designated as 1.

An advantage of PLSR-DA is that the results obtained can be easily represented in the form of two different plots, the score and loading plots. Score plots represent a projection of the samples onto the principal components and shows the distribution of the samples in the classification model and their relationship to one another. Loading plots display correlations between the variables present in the data set.

It is usually recommended to use PLS-DA as a starting point for the classification problem due to its ability to handle collinear data, and the property of PLSR as a dimension reduction technique. Once this purpose has been satisfied, it is possible to use other methods such as Linear discriminant analysis, LDA, that has been shown to be effective in extracting further information, Indahl et al. (1999, Chem. and Intell. Lab. Syst., 49, p 19-31). This approach is based on first decomposing the data using PLS-DA, and then using the scores vectors (instead of the original variables) as input to LDA. Further details on LDA can be found in Duda and Hart (Classification and Scene Analysis, 1973, Wiley, USA).

The next step following model building is of model validation. This step is considered to be amongst the most important aspects of multivariate analysis, and tests the “goodness” of the calibration model which has been built. In this work, a cross validation approach has been used for validation. In this approach, one or a few samples are kept out in each segment while the model is built using a full cross-validation on the basis of the remaining data. The samples left out are then used for prediction/classification. Repeating the simple cross-validation process several times holding different samples out for each cross-validation leads to a so-called double cross-validation procedure. This approach has been shown to work well with a limited amount of data, as is the case in some of the Examples described here. Also, since the cross validation step is repeated several times the dangers of model bias and overfitting are reduced.

Once a calibration model has been built and validated, genes exhibiting an expression pattern that is most relevant for describing the desired information in the model can be selected by techniques described in the prior art for variable selection, as mentioned elsewhere. Variable selection will help in reducing the final model complexity, provide a parsimonious model, and thus lead to a reliable model that can be used for prediction. Moreover, use of fewer genes for the purpose of providing diagnosis will reduce the cost of the diagnostic product. In this way informative probes which would bind to the genes of relevance may be identified.

We have found that after a calibration model has been built, statistical techniques like Jackknife (Effron, 1982, The Jackknife, the Bootstrap and other resampling plans. Society for Industrial and Applied mathematics, Philadelphia, USA), based on resampling methodology, can be efficiently used to select or confirm significant variables (informative probes).

The approximate uncertainty variance of the PLS regression coefficients B can be estimated by:

$${S}^{2} B = {\sum }_{m = 1}^{M} {( ( B - {B}_{m} ) g )}^{2}$$

where
S²B=estimated uncertainty variance of B;
B=the regression coefficient at the cross validated rank A using all the N objects;
Bₘ=the regression coefficient at the rank A using all objects except the object(s) left out in cross validation segment m; and
g=scaling coefficient (here: g=1).

In our approach, Jackknife has been implemented together with cross-validation. For each variable the difference between the B-coefficients Bᵢ in a cross-validated sub-model and Bₜₒₜ for the total model is first calculated. The sum of the squares of the differences is then calculated in all sub-models to obtain an expression of the variance of the Bᵢ estimate for a variable. The significance of the estimate of Bᵢ is calculated using the t-test. Thus, the resulting regression coefficients can be presented with uncertainty limits that correspond to 2 Standard Deviations, and from that significant variables are detected.

No further details as to the implementation or use of this step are provided here since this has been implemented in commercially available software, The Unscrambler, CAMO ASA, Norway. Also, details on variable selection using Jackknife can be found in Westad & Martens (2000, J. Near Inf. Spectr., 8, p 117-124).

The following approach can be used to select informative probes from a gene expression data set:

a) keep out one unique sample (including its repetitions if present in the data set) per cross validation segment;

b) build a calibration model (cross validated segment) on the remaining samples using PLSR-DA;

c) select the significant genes for the model in step b) using the Jackknife criterion;

d) repeat the above 3 steps until all the unique samples in the data set are kept out once (as described in step a). For example, if 75 unique samples are present in the data set, 75 different calibration models are built resulting in a collection of 75 different sets of significant probes;

e) select the most significant variables using the frequency of occurrence criterion in the generated sets of significant probes in step d). For example, a set of probes appearing in all sets (100%) are more informative than probes appearing in only 50% of the generated sets in step d).

Once the informative probes for a disease have been selected, a final model is made and validated. The two most commonly used ways of validating the model are cross-validation (CV) and test set validation. In cross-validation, the data is divided into k subsets. The model is then trained k times, each time leaving out one of the subsets from training, but using only the omitted subset to compute error criterion, RMSEP (Root Mean Square Error of Prediction). If k equals the sample size, this is called “leave-one-out” cross-validation. The idea of leaving one or a few samples out per validation segment is valid only in cases where the covariance between the various experiments is zero. Thus, one sample at-a-time approach can not be justified in situations containing replicates since keeping only one of the replicates out will introduce a systematic bias in our analysis. The correct approach in this case will be to leave out all replicates of the same samples at a time since that would satisfy assumptions of zero covariance between the CV-segments.

The second approach for model validation is to use a separate test-set for validating the calibration model. This requires running a separate set of experiments to be used as a test set. This is the preferred approach given that real test data are available.

The final model is then used to identify a disease, condition or stage thereof in test samples. For this purpose, expression data of selected informative genes is generated from test samples and then the final model is used to determine whether a sample belongs to a diseased or non-diseased class or has a condition or stage thereof.

Thus viewed from a yet further aspect the present invention provides a method of identifying probes useful for diagnosing or identifying or monitoring a disease or condition or stage thereof in an organism, comprising the steps of:
a) immobilizing a set of oligonucleotide probes, preferably as described hereinbefore, on a solid support;
b) isolating mRNA from a sample of a normal organism (normal sample), which may optionally be reverse transcribed to cDNA;
c) isolating mRNA from a sample from an organism, corresponding to the sample and organism of step (b), which is known to have said disease or condition or a stage thereof (diseased sample), which may optionally be reverse transcribed to cDNA;
d) hybridizing the mRNA or cDNA of steps (b) and (c) to said set of immobilized oligonucleotide probes of step (a); and
e) assessing the amount of mRNA or cDNA hybridizing to each of said oligonucleotide probes to determine the level of gene expression of genes to which said oligonucleotide probes bind in said normal and diseased samples to generate a gene expression data set for each sample;
f) normalizing and standardizing said data set of step (e);
g) constructing a calibration model for classification, preferably using the statistical techniques Partial Least Squares Discriminant Analysis (PLS-DA) and Linear Discriminant Analysis (LDA);
h) performing JackKnife analysis and identifying those oligonucleotide probes which are required for classification of said disease and normal samples into their respective groups.

Preferably a model for classification purposes is generated by using the data relating to the probes identified according to the above described method. Preferably the sample is as described previously. Preferably the oligonucleotides which are immobilized in step (a) are randomly selected as described below or are the probes as described hereinbefore. Such oligonucleotides may be of considerable length, e.g. if using cDNA (which is encompassed within the scope of the term “oligonucleotide”). The identification of such cDNA molecules as useful probes allows the development of shorter oligonucleotides which reflect the specificity of the cDNA molecules but are easier to manufacture and manipulate.

The above described model may then be used to generate and analyse data of test samples and thus may be used for the diagnostic methods of the invention. In such methods the data generated from the test sample provides the gene expression data set and this is normalized and standardized as described above. This is then fitted to the calibration model described above to provide classification.

The method described herein can also be used to simultaneously select informative probes for several related and unrelated diseases or conditions. Depending upon which diseases or conditions have been included in the calibration or training set, informative probes can be selected for the said diseases or conditions. The informative probes selected for one disease or condition may or may not be similar to the informative probes selected for another disease or condition of interest. It is the pattern with which the selected genes are expressed in relation to each other during a disease, condition, or stage thereof, that determines whether or not they are informative for the disease, condition or stage thereof.

In other words, informative genes are selected based on how their expression correlates with the expression of other selected informative genes under the influence of responses generated by the disease, condition or stage thereof under investigation. In examples 1 and 2 provided hereinafter, 139 informative probes were selected for breast cancer diagnosis and 182 probes were selected for Alzheimer's disease diagnosis by training the gene expression data set of genes representing 1435 or 758 randomly picked cDNA clones for breast cancer/non breast cancer samples, or Alzheimer/non-Alzheimer samples, respectively. Among the probes selected for breast cancer and Alzheimer, about 10 probes were informative both for breast cancer and Alzheimer disease diagnosis.

For the purpose of isolating informative probes or identifying several related and unrelated diseases, conditions and stages thereof simultaneously, the gene expression data set must contain the information on how genes are expressed when the subject has a particular disease, condition or stage thereof under investigation.

The data set is generated from a set of healthy or diseased samples, where a particular sample may contain the information of only one disease, condition or stages thereof or may also contain information about multiple diseases, conditions or stages thereof. For example, if the isolation of informative probes for Alzheimer disease, breast cancer and diabetes is sought, whole blood samples can be obtained from an Alzheimer patient who has breast cancer and diabetes. Hence, the method also teaches an efficient experimental design to reduce the number of samples required for isolating informative probes by selecting samples representing more than one disease, condition or stage thereof.

As mentioned previously, in view of the high information content of most transcripts, the identification and selection of informative probes for use in diagnosing, monitoring or identifying a particular disease, condition or stage thereof may be dramatically simplified. Thus the pool of genes from which a selection may be made to identify informative probes may be radically reduced.

Unlike, in prior art technologies where informative probes are selected from a population of thousands of genes that are being expressed in a cell, like in microarray, in the method described herein, the informative probes are selected from a limited number of randomly obtained genes. For example, from a population of 1435 cDNA clones, randomly picked from a human whole blood cDNA library, we were able to select 139 informative probes for breast cancer diagnosis (see Example 1 and Table 2).

Thus in a preferred aspect of the above mentioned method of identifying probes useful for diagnosing or identifying or monitoring a disease or condition or stage thereof in an organism, said set of oligonucleotides which are immobilized in step (a) are randomly selected from a larger set of oligonucleotides, e.g. from a cDNA library or other oligonucleotide pool, which may be, but is preferably not selected from the set provided herein. Preferably said larger set comprises oligonucleotides which correspond to moderately or highly expressed genes. Thus preferably in methods of the invention, the set of oligonucleotides according to the invention are replaced with a set of oligonucleotides which are randomly selected, e.g. from commercially available oligonucleotide or cDNA libraries.

As referred to herein “random” refers to selection which is not biased based on the extent of information carried by the transcripts in relation to the disease, condition or organism under study, ie. without bias towards their likely utility as informative probes. Whilst a random selection may be made from a pool of transcripts (or related products) which have been biased, e.g. to highly or moderately expressed transcripts, preferably random selection is made from a pool of transcripts not biased or selected by a sequence-based criterion. The larger set may therefore contain oligonucleotides corresponding to highly and moderately expressed genes, or alternatively, may be enriched for those corresponding to the highly and moderately expressed genes.

Random selection from highly and moderately expressed genes can be achieved in a wide variety of ways. A strategy used in this work, but not limiting in itself involves randomly picking a significant number of cDNA clones from a cDNA library constructed from a biological specimen under investigation. Since, in a cDNA library, the cDNA clones corresponding to transcripts present in high or moderate amount are more frequently present than transcripts corresponding to cDNA present in low amount, the former will tend to be picked up more frequently than the latter. A pool of cDNA enriched for those corresponding to highly and moderately expressed genes can be isolated by this approach.

To identify genes that are expressed in high or moderate amount among the isolated population for use in methods of the invention, the information about the relative level of their transcripts in samples of interest can be generated using several prior art techniques. Both non-sequence based methods, such as differential display or RNA fingerprinting, and sequence-based methods such as microarrays or macroarrays can be used for the purpose. Alternatively, specific primer sequences for highly and moderately expressed genes can be designed and methods such as quantitative RT-PCR can be used to determine the levels of highly and moderately expressed genes. Hence, a skilled practitioner may use a variety of techniques which are known in the art for determining the relative level of mRNA in a biological sample.

Especially preferably the sample for the isolation of mRNA in the above described method is as described previously and is preferably not from the site of disease and the cells in said sample are not disease cells and have not contacted disease cells.

The following examples are given by way of illustration only in which the Figures referred to are as follows:

FIG. 1 shows the effect of Direct Standardization (DS) on the Alzheimer data measured in two different series of experiments in which AD denotes Alzheimer's samples and A, B are non-Alzheimer's samples. The samples in both series have been labelled systematically as (xx_{—}7/xx_{—}8), whereas the corrected samples from series 8 (in b,c,d) have been labelled as (xx_{—c}), thus, for example, AD2-7 denotes Alzheimer disease sample number 2 in experiment series 7. The circled spots represent the samples chosen as the transfer samples. The connecting lines in figures b,c,d show the proximity of the replicated samples after applying DS. The dashed lines in figures a,c,d represent the decision boundary separating the classes. These lines have not been drawn on the basis of any statistical criteria, but serve the purpose of visually separating the classes. All the four figures show scores plot (PC1-PC2) from PCA analysis based on (a) non-standardized data, (b) scores plot after direct standardization using 3 transfer samples, (c) scores plot after direct standardization using 4 transfer sample, (d) scores plot after direct standardization using 8 transfer samples;

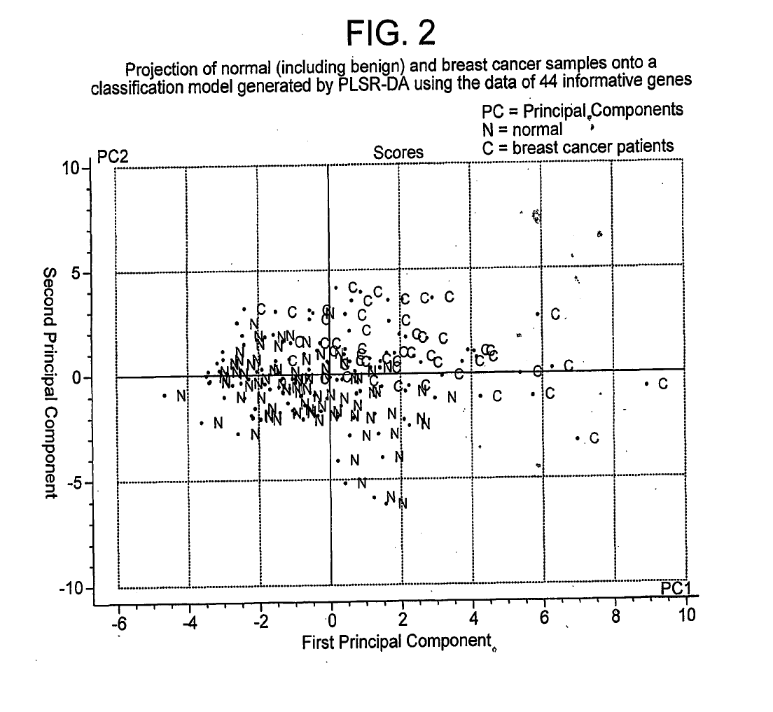

FIG. 2 shows the projection of normal (including benign) and breast cancer samples onto a classification model generated by PLSR-DA using the data of 44 informative genes, in which PC is the principal components and N and C are normal and breast cancer samples, respectively;

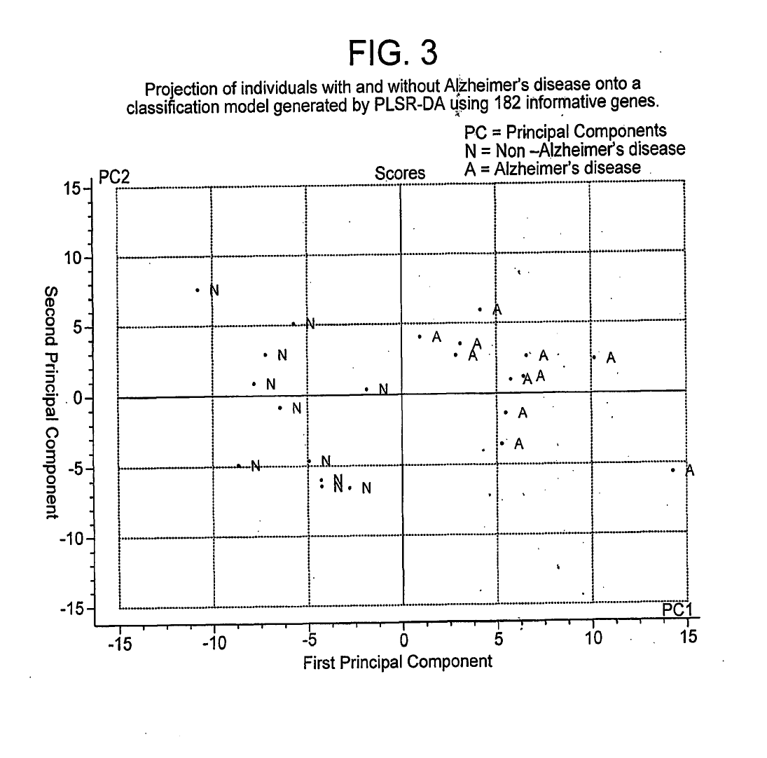

FIG. 3 shows the projection of individuals with and without Alzheimer's disease onto a classification model generated by PLSR-DA using 182 informative genes;

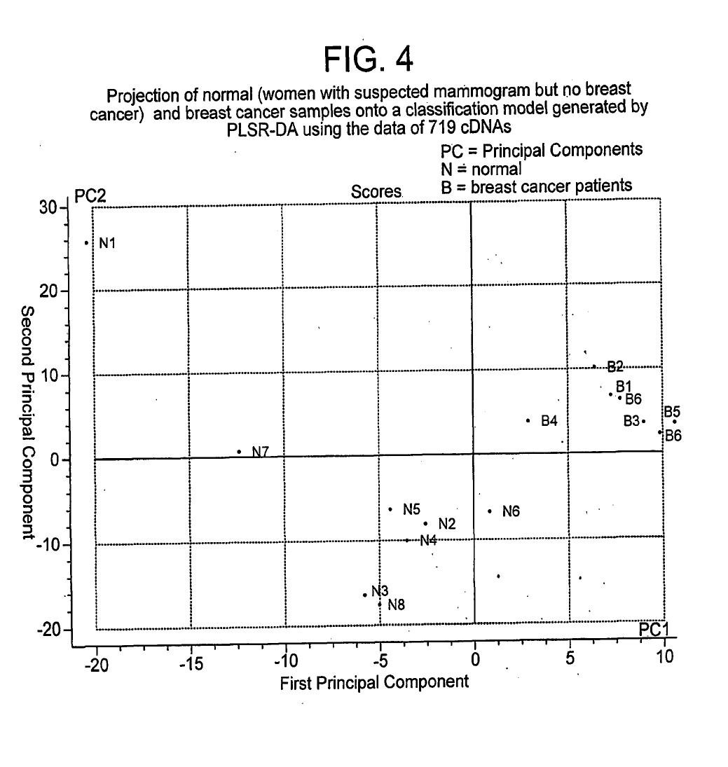

FIGS. 4, 6 and 8 show projection plots as FIG. 2 in which the classification model is generated using 719, 111 and 345 cDNAs, respectively, wherein PC is the principal components, N denotes normal and B denotes breast cancer samples;

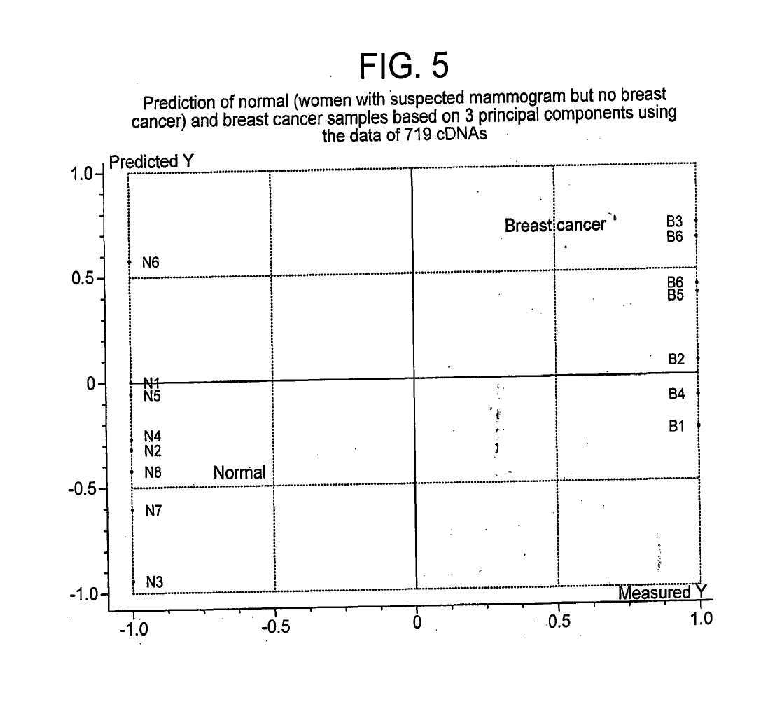

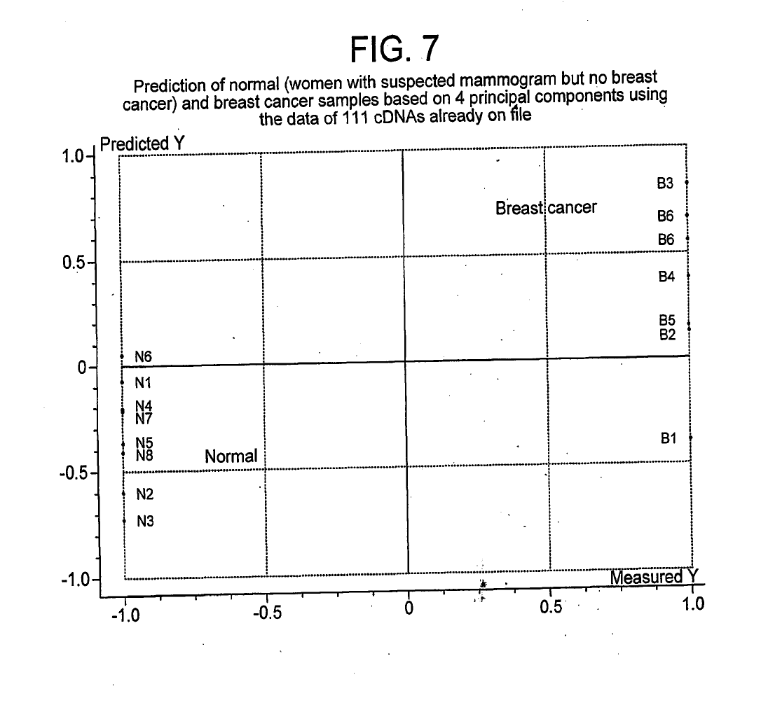

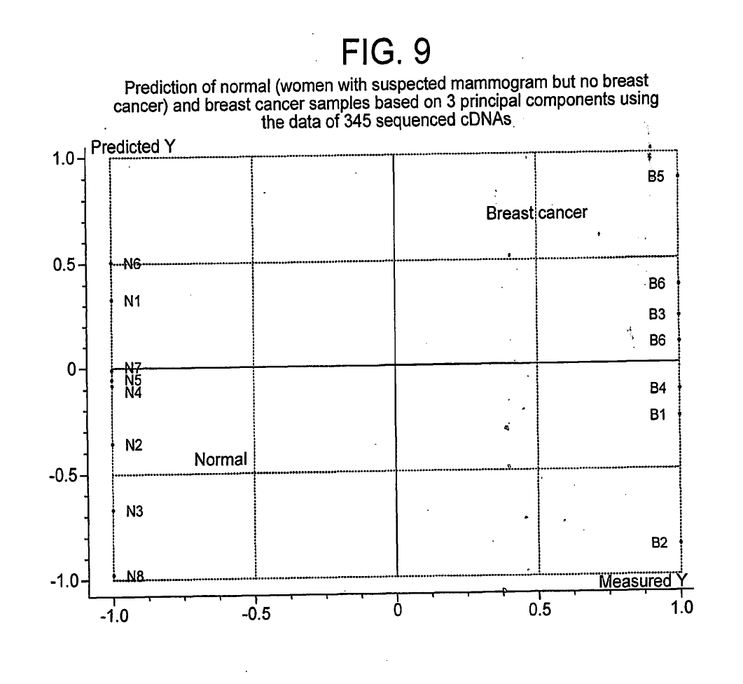

FIGS. 5, 7 and 9 show prediction plots based on 3 principal components using the data of 719, 111 and 345 cDNAs, respectively;

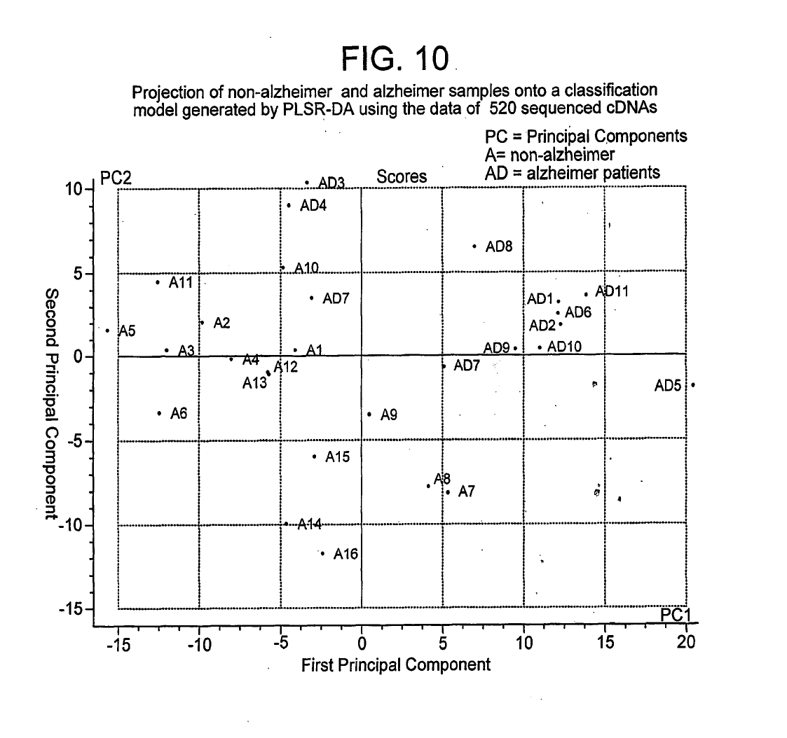

FIG. 10 shows a projection plot as FIG. 3 in which the classification model is generated using 520 cDNAs; and

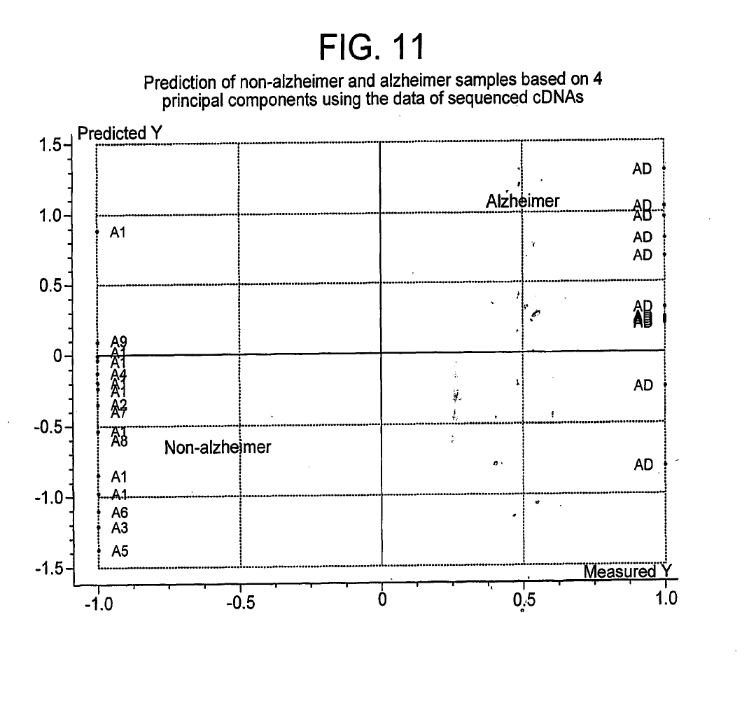

FIG. 11 is the prediction plot corresponding to FIG. 10.

### EXAMPLE 1

### Diagnosis of Breast Cancer

### Methods

Whole blood was obtained from the arms of breast cancer patients and patients with benign tumours (Ullev\dot{a}l and Haukland hospitals in Norway). All of the patients with breast cancer had a malignant tumour of the breast (disease samples). Healthy blood was collected from the above two hospitals, or collected at a Health station at As, Norway or at DiaGenic AS, Norway, from the arms of female donors with no reported signs of breast cancer. The blood from healthy individuals or with benign tumours comprise the normal samples. The blood was either collected in tubes containing EDTA and stored immediately at −80° C. or was collected in PAXgene tubes and stored for 12-24 hours at room temperature before finally storing them at −80° C. before use. Further details of the breast cancer and benign tumour patients from which blood was taken is provided in Table 5.

mRNA was isolated from the blood of the 29 breast cancer patients and 46 normal donors and used to prepare labelled probes by reverse transcribing in the presence of α³³P-dATP. The first strand cDNA of the normal and diseased samples was bound, separately to 1435 cDNA clones immobilized on a solid support (nylon membrane).

These cDNA clones were randomly picked, without any prior knowledge of their gene sequences, from a cDNA library constructed using whole blood of 550 healthy individuals (Clontech, Palo Alto, USA). These methods were conducted as follows.

For amplification of inserts, bacterial clones were grown in microtiter plates containing 150 μl LB with 50 μg/ml carbenicillin, and incubated overnight with agitation at 37° C. To lyse the cells, 5 μl of each culture were diluted with 50 μl H2O and incubated for 12 min. at 95° C. Of this mixture, 2 μl were subjected to a PCR reaction using 20 pmoles of M13 forward and reverse primer in presence of 1.5 mM MgCl₂. PCR reactions were performed with the following cycling protocol: 4 min. at 95° C., followed by 25 cycles of 1 min. at 94° C., 1 min. at 60° C. and 3 min. at 72° C. either in a RoboCycler® Temperature Cycler (Stratagene, La Jolla, USA) or DNA Engine Dyad Peltier Thermal Cycler (MJ Research Inc., Waltham, USA). The amplified products were denatured by incubating with NaOH (0.2 M, final concentration) for 30 min. and spotted onto Hybond-N+ membranes (Amersham Pharmacia Biotech, Little Chalfont, UK), using MicroGrid II workstation according to the manufacturer's instructions (BioRobotics Ltd, Cambridge England). The immobilized cDNAs were fixed using a UV cross-linker (Hoefer Scientific Instruments, San Francisco, USA).

In addition to the 1435 cDNAs, the printed arrays also contained controls for assessing background level, consistency and sensitivity of the assay. These were spotted at multiple positions and included controls such as PCR mix (without any insert); positive and negative controls of SpotReport™ 10 array validation system (Stratagene, La Jolla, USA) and cDNAs corresponding to constitutively expressed genes such as b-actin, g-actin, GAPDH, HOD and cyclophilin. Also, oligonucleotides corresponding to SIX1, b-tubulin, TRP-2, MDM2, Myosin Light C, CD44, Maspin, Laminin, and SRP 19 were included to detect disseminated cancer cells.

The total RNA from blood collected in EDTA tubes was purified using Trizol LS Reagent protocol (Invitrogen/Life Technologies). From blood contained in PAXgene tubes, the total RNA was purified according to the supplier's instructions (PreAnalytiX, Hombrechtikon, Switzerland). Contaminating DNA was removed from the isolated RNA by DNAase I treatment using DNA-free kit (Ambion, Inc. Austin, USA). RNA quality was determined visually by inspecting the integrity of 28S and 18S ribosomal bands following agarose gel electrophoresis. The concentration and purity of extracted RNA was determined by measuring the absorbance at 260 nm and 280 nm. mRNA was isolated from the total RNA using Dynabeads as per the supplier's instructions (Dynal AS, Oslo, Norway).

Labelling and hybridization experiments were performed in batches. The number of samples assayed in each batch varied from six to nine. In the case of samples that were assayed more than once (replicates), aliquots derived from the same mRNA pool were used for probe synthesis. For probe synthesis, aliquots of mRNA corresponding to 4-5 mg of total RNA were mixed together with oligodT_{25NV} (0.5 mg/ml) and mRNA spikes of SpotReport™ 10 array validation system (10 pg; Spike 2, 1 pg), heated to 70° C. to remove secondary structures, and then chilled on ice. Probes were prepared in 35 μl reaction mixes by reverse transcription in the presence of 50 μCi [α³³P] dATP, 3.5 μM dATP, 0.6 mM each of dCTP, dTTP, dGTP, 200 units of SuperScript reverse transcriptase (Invitrogen, LifeTechnologies) and 0.1 M DTT, labelling for 1.5 hr at 42° C. Following synthesis, the enzyme was deactivated for 10 min. at 70° C. and mRNA removed by incubating the reaction mix for 20 min. at 37° C. in 4 units of Ribo H (Promega, Madison USA). Unincorporated nucleotides were removed using ProbeQuant G 50 Columns (Amersham Biosciences, Piscataway, USA).

Prior to hybridization, the membranes were equilibrated in 4×SSC for 2 hr at room temperature and prehybridized overnight at 65° C. in 10 ml prehybridisation solution (4×SSC, 0.1 M NaH₂PO₄, 1 mM EDTA, 8% dextran sulphate, 10×denhardt's solution, 1% SDS). Freshly prepared probes were added to 5 ml of the same prehybridisation solution, and hybridization continued overnight at 65° C. The membranes were washed at 65° C. at increasing stringency (2×30 min. each in 2×SSC, 0.1% SDS; 1×SSC, 0.1% SDS; 0.1×SSC, 0.1% SDS) to remove unspecific signals.

The amount of labelled first strand cDNA binding to each spot was assessed and quantified using a PhosphoImager to generate a gene expression data set. The data was generated using Phoretix software version 3 (Non Linear Dynamics, England). Background subtraction was performed on the generated data by subtracting the median of the line of pixels around each spot outline from the total intensity obtained from the respective spots.

The background-subtracted data was then normalized and transformed by selecting out 50 lowest and 50 maximum signals from each membrane. This step was to exclude genes that were expressed with a high degree of variance. Since the genes varied from membrane to membrane, the expression data from 497 genes were removed from the data set. The values for the remaining 938 genes were then normalised by using different approaches such as external controls, dividing each spot by the median intensity of the observed signal in the respective membrane, range normalizing the data from each membrane, and then log transforming the data obtained.

The processed data obtained above was then used to isolate the informative probes by:

a) keeping one unique sample (including all repetitions of the selected sample) out per cross validation segment;

b) building a calibration model (cross validated) on the remaining samples using PLSR-DA;

c) selecting the set of significant genes for the model in step b using the Jackknife criterion;

d) repeating steps a), b) and c) until all the unique samples were kept out once (hence, in all 75 different calibration models were built (after repeating step b) 75 times), resulting in 75 different sets of significant probes (after repeating step c) 75 times));

e) selecting significant variables using the frequency of occurrence criterion amongst the 75 different sets of significant probes.

The selected informative probes based on occurrence criterion were used to construct a classification model. The result of the classification model based on probes appearing in at least 90% of the generated sets after the step of isolating informative probes as described above is shown in FIG. 2 in which it is seen that the expression pattern of these genes was able to classify most women with breast cancer and women with no breast cancer into distinct groups. In this figure PC1 and PC2 indicate the two principal components statistically derived from the data which best define the systemic variability present in the data. This allows each sample, and the data from each of the informative probes to which the sample's labelled first strand cDNA was bound, to be represented on the classification model as a single point which is a projection of the sample onto the principal components—the score plot.

The ability of the generated model, based on isolated informative probes, to predict future samples was determined by the double cross-validation approach. The performance of the diagnostic test for breast cancer based on the occurrence criterion is presented in Table 6.

Correct prediction of most breast cancer cells was achieved. These included all three samples obtained from women with ductal carcinoma in situ (DCIS), 11/15 samples obtained from women with stage I breast cancer, all five samples obtained from women with stage II breast cancer, and one of two samples obtained from women with stage III breast cancer. Interestingly, two correctly predicted stage I samples were obtained from women having a tumour size of <5 mm in diameter.

The model also correctly predicted the class of most non-cancer samples (41/46), including those that were obtained from women with non-cancerous breast abnormalities.

Confirmation that the gene transcripts are not from cells which are disseminated disease cells has been confirmed by several lines of evidences. Firstly, the informative genes were expressed constitutively at high or moderate levels in blood cells of women irrespective of whether they had cancer or not. Secondly, in the assay described in this Example, in order to identify transcripts, at least 720 disseminated cells in blood samples would be required. Since, the average number of disseminated cells present in blood during different stages of breast cancer is much lower (organ confined breast cancer, 0.8 cells per ml; invasive breast cancer spread to lymph nodes only, 2.4 cells per ml; and metastatic breast cancer, 6 cells per ml; SD>100%) (29), we believe that the signals being detected originated from peripheral blood cells and could not have originated from disseminated cells. Thirdly, we were not able to detect any signal from the eight cancer markers known to have elevated expression in malignant cancer cells, including cancer cells that are disseminated in the blood.

### EXAMPLE 2

### Diagnosis of Alzheimer's Disease

Similar experiments were conducted with samples from Alzheimer's patients. In this method 7 patients diagnosed with Alzheimer's Disease at the Memory Clinic at Ullev\dot{a}l University Hospital were used in the trial. The patients were confirmed as having Alzheimer's disease based on the following criteria:
A standardized interview with a care-giver using IQCODE, an ADL scale and a scale measuring behaviour of the patient (Green scale).
Neuropsychological evaluation using MMSE, Clock drawing test, Trailmaking test A and B (TMT A and B), Kendrick object learning test (visual memory test), part of the Wechsler battery and Benton test.
A psychiatric evaluation using scales for detection of depression, MADRS for interviewing the patient and Cornell scale for interviewing the care-giver.
A physical examination.
Laboratory tests of blood samples to rule out other diseases.
CT scan of the brain.
SPECT of the brain.

The mean age of the patients was 72.3 with an age range of 69-76. The mean MMSE score was 22.0 (the maximum score attainable being 30).

Six age-matched individuals without diagnosed Alzheimer's disease were used as a control. All had been tested with MMSE and had a minimum score of 28 (mean: 28.4). The mean age of the normal control group was 73.0 and the age range 66-81. A sample from a 16-year old individual, with a consequent minimal chance of having Alzheimer's disease, was also included as an additional control.

Using the methods described above (except that hybridization to 758 rather than 1435 cDNA clones was performed), informative probes were selected based on occurrence criterion and used to construct a classification model. The results of the classification model based on probes appearing at least once in the generated sets after the method to isolate informative probes as described above is shown in FIG. 3 in which it will be seen that the expression pattern of these genes was able to classify individuals with or without Alzheimer's disease into distinct groups. In this Figure PC1 and PC2 indicate the 2 principal components statistically derived from the data which define the systematic variability present in the data. This allows each sample, and the data from each of the informative probes to which the samples' cDNA was bound, to be represented on the classification model as a single point which is a projection of the sample onto the principal components—the score plot.

The ability of the generated model, based on isolated informative probes, to predict future samples was determined by the double cross-validation. The performance of the diagnostic test for Alzheimer's disease is presented in Table 7.

### APPENDIX A

### Partial Least Squares Regression (PLSR)

Let a multivariate regression model be defined as:

Y=XB+F

where
X a N×P matrix with N predictor variables (genes);
Y (N×J) being the J predicted variables. In our case Y represents a matrix containing dummy variables;
B is a matrix of regression coefficients; and
F is a N×J matrix of residuals.

The structure of the PLSR model can be written as:

X=TP^{T}+E_{A}, and

Y=TQ^{T}+F_{A}, where

where
T (N×A) is a matrix of score vectors which are linear combinations of the x-variables;
P (P×A) is a matrix with the x-loading vectors pₐ as columns;
Q (J×A) is a matrix with the y-loading vectors qₐ as columns;
Eₐ (N×P) is the matrix for X after A factors; and
Fₐ (N×J) is the matrix for Y after A factors.

The criterion in PLSR is to maximize the explained covariance of [X, Y]. This is achieved by the loading weights vector wₐ₊₁, which is the first eigenvector of Eₐ^{T}FₐFₐ^{T}Eₐ (Eₐ and Fₐ are the deflated X and Y after a factors or PLS components).

The regression coefficients are given by:

B=W(P^{T}W)⁻¹Q^{T}

A PLSR model with full rank, i.e. maximum number of components, is equivalent to the MLR solutions. Further details on PLSR can be found in Marteus & Naes, 1989, Multivariate Calibration, John Wiley & Sons, Inc., USA and Kowalski & Seasholtz, 1991, supra.

### EXAMPLE 3

### Validation of Example 1, Diagnosis of Breast Cancer

The results in Example 1 were validated by using the informative probes identified in Example 1 on new beast cancer and control samples.

### Methods

The methods, essentially as described in Example 1, were used. Blood was taken from patients as described in Table 8. However, blood was collected in PAXgene tubes and the first strand labelled cDNAs were hybridized to 719 cDNAs spotted on nylon membranes along with other controls as described in Example 1. After background subtraction using control spots, the data of each membrane was normalized using the inter quantile range.

The data was analysed as described in Example 1 and the model validated by cross validation.

The 719 cDNAs which were spotted are a subset of the cDNAs spotted in Example 1 and include 111 cDNAs described in Table 2 and which were found to be informative in Example 1.

### Results

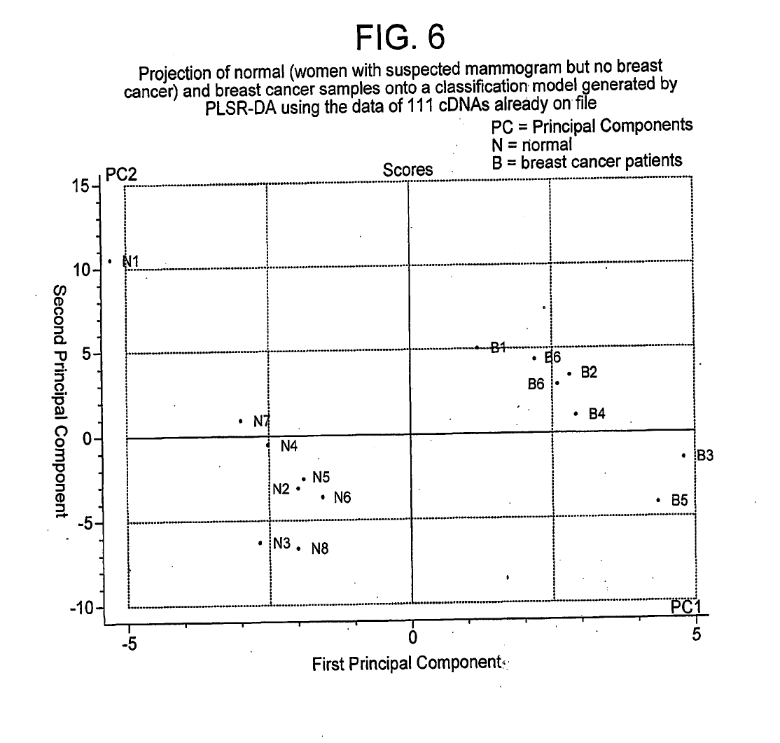

The results are shown in FIGS. 4 to 9. FIGS. 4, 6 and 8 are projection plots similar to FIG. 2 and show the projection of normal and breast cancer patients' samples onto a classification model generated using all 719 cDNA. FIG. 6 is similar but uses a classification model generated with the 111 probes common to Example 1.

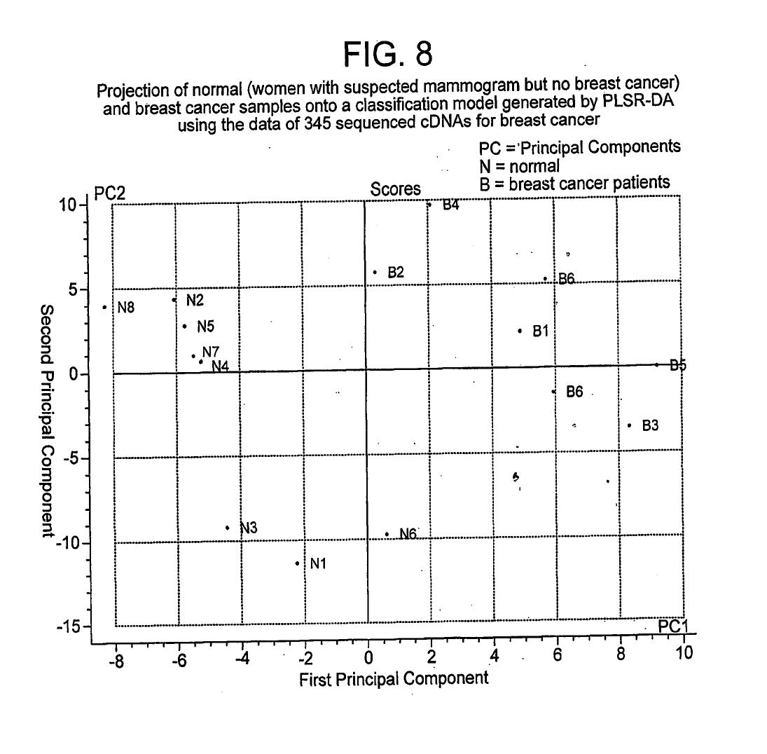

FIG. 8 uses the 345 sequences of the 719 for which sequence information is provided herein. In each case classification of normal and breast cancer groups was possible. FIGS. 5, 7 and 9 show prediction plots which reflect the ability of the generated models to correctly diagnose breast cancer. In the 3 prediction plots shown, the disease samples appear on the x axis at +1 and the non-disease samples appear at −1. The y axis represents the predicted class membership. During prediction, if the prediction is correct, disease samples should fall above zero and non-disease samples should fall below zero. In each case almost all samples are correctly predicted.

### EXAMPLE 4

### Validation of Example 2, Diagnosis of Alzheimers

The results in Example 2 were validated by using the informative probes identified in Example 2 on new Alzheimer's patient samples.

### Methods

The methods, essentially as described in Example 2, were used. Twelve female patients diagnosed with Alzheimer's disease at the Memory Clinic at Ullev\dot{a}l University Hospital who were confirmed as having Alzheimer's disease based on the criteria of Example 2 were used in the trial. The mean age of the patients was 72.3 with an age range of 66-83. The mean MMSE score was 22.0 (the maximum score attainable being 30).

Sixteen age-matched female individuals without diagnosed Alzheimer's disease were used as the normal control group. All had been tested with MMSE and had a minimum score of 29. The mean age of the normal control group was 74.0 and the age range 66-86.

After transfer of the blood to PAXgene tubes, total mRNA was isolated from the blood of the Alzheimer's disease and from the control group donors according to the manufacturers's instructions (PreAnalytiX, Hombrechtikon, Switzerland). The isolated mRNA was labelled during reverse transcription in the presence of α³³P-dATP, yielding a labelled first strand cDNA. Hybridization was performed as described previously onto 730 cDNA clones picked from a cDNA library from whole blood of 550 healthy individuals without knowledge of the gene sequence of the random cDNA clones.

### Results

The results are shown in FIGS. 10 and 11. FIG. 10 is a projection plot generated using 520 probes which have been sequenced. FIG. 11 is a prediction plot and shows correct prediction of almost all samples.

TABLE 1a
List of probes informative for disease diagnosis

| | | No. of | SEQ ID NO: in |
|---|---|---|---|
| | Clone ID | nucleotides | sequence listing |
| 1 | I-24 | 373 | 11 |
| 2 | I-28 | 564 | 13 |
| 3 | I-30 | 622 | 398 |
| 4 | I-34 | 554 | 15 |
| 5 | I-54 | 155 | 399 |
| 6 | I-58 | 554 | 24 |
| 7 | II-03 | 622 | 34 |
| 8 | II-05 | 628 | 35 |
| 9 | II-06 | 527 | 36 |
| 10 | II-10 | 329 | 39 |
| 11 | II-24 | 534 | 47 |
| 12 | II-25 | 444 | 48 |
| 13 | II-26 | 566 | 49 |
| 14 | II-33 | 523 | 55 |
| 15 | II-34 | 566 | 56 |
| 16 | II-41 | 534 | 60 |
| 17 | II-42 | 512 | 61 |
| 18 | II-57 | 505 | 73 |
| 19 | II-61 | 596 | 77 |
| 20 | II-69 | 387 | 85 |
| 21 | II-70 | 420 | 86 |
| 22 | II-75 | 535 | 91 |
| 23 | II-84 | 577 | 99 |
| 24 | II-87 | 552 | 100 |
| 25 | II-88 | 606 | 101 |
| 26 | II-94 | 329 | 104 |
| 27 | III-02 | 747 | 107 |
| 28 | III-06 | 682 | 109 |
| 29 | III-08 | 536 | 111 |
| 30 | III-13 | 615 | 115 |
| 31 | III-20 | 479 | 401 |
| 32 | III-23 | 694 | 119 |
| 33 | III-26 | 476 | 122 |
| 34 | III-35 | 551 | 130 |
| 35 | III-39 | 224 | 131 |
| 36 | III-40 | 349 | 132 |
| 37 | III-43 | 382 | 499 |
| 38 | III-44 | 382 | 134 |
| 39 | III-53 | 390 | 142 |
| 40 | III-56 | 109 | 144 |
| 41 | III-57 | 374 | 145 |
| 42 | III-61 | 521 | 148 |
| 43 | III-63 | 575 | 150 |
| 44 | III-74 | 502 | 155 |
| 45 | III-80 | 585 | 158 |
| 46 | III-85 | 516 | 161 |
| 47 | III-89 | 660 | 165 |
| 48 | IV-14 | 545 | 275 |
| 49 | IV-15 | 628 | 402 |
| 50 | IV-26 | 494 | 403 |
| 51 | IV-31 | 268 | 278 |
| 52 | IV-32 | 569 | 279 |
| 53 | IV-53 | 362 | 498 |
| 54 | IV-69 | 286 | 4 |
| 55 | IV-80 | 579 | 291 |
| 56 | IX-10 | 641 | 314 |
| 57 | IX-38 | 583 | 317 |
| 58 | IX-39 | 424 | 318 |
| 59 | IX-48 | 626 | 319 |
| 60 | IX-77 | 556 | 325 |
| 61 | V-03 | 496 | 296 |
| 62 | V-04 | 397 | 297 |
| 63 | V-07 | 293 | 298 |
| 64 | V-11 | 599 | 404 |
| 65 | V-12 | 498 | 301 |
| 66 | V-55 | 464 | 501 |
| 67 | V-80 | 260 | 311 |
| 68 | VI-04 | 122 | 339 |
| 69 | VI-07 | 405 | 1 |
| 70 | VI-12 | 667 | 341 |
| 71 | VI-14 | 642 | 343 |
| 72 | VI-20 | 115 | 346 |
| 73 | VI-23 | 634 | 347 |
| 74 | VI-48 | 626 | 355 |
| 75 | VI-50 | 585 | 356 |
| 76 | VI-53 | 560 | 357 |
| 77 | VI-55 | 509 | 359 |
| 78 | VI-70 | 550 | 2 |
| 79 | VI-74 | 655 | 365 |
| 80 | VI-76 | 582 | 367 |
| 81 | VI-87 | 595 | 370 |
| 82 | VI-88 | 651 | 371 |
| 83 | VI-95 | 230 | 374 |
| 84 | VII-03 | 412 | 411 |
| 85 | VII-15 | 439 | 414 |
| 86 | VII-19 | 580 | 171 |
| 87 | VII-21 | 671 | 173 |
| 88 | VII-32 | 457 | 179 |
| 89 | VII-36 | 209 | 182 |
| 90 | VII-39 | 541 | 183 |
| 91 | VII-42 | 502 | 186 |
| 92 | VII-43 | 316 | 187 |
| 93 | VII-46 | 631 | 190 |
| 94 | VII-47 | 526 | 415 |
| 95 | VII-48 | 613 | 416 |
| 96 | VII-59 | 565 | 199 |
| 97 | VII-63 | 98 | 201 |
| 98 | VII-66 | 362 | 204 |
| 99 | VII-72 | 595 | 206 |
| 100 | VII-73 | 522 | 207 |
| 101 | VII-76 | 624 | 209 |
| 102 | VII-77 | 692 | 418 |
| 103 | VII-80 | 338 | 210 |
| 104 | VII-81 | 556 | 211 |
| 105 | VII-90 | 576 | 216 |
| 106 | VII-91 | 341 | 217 |
| 107 | VII-93 | 379 | 219 |
| 108 | VIII-09 | 598 | 221 |
| 109 | VIII-20 | 419 | 229 |
| 110 | VIII-28 | 511 | 235 |
| 111 | VIII-29 | 592 | 236 |
| 112 | VIII-30 | 572 | 237 |
| 113 | VIII-31 | 482 | 238 |
| 114 | VIII-32 | 545 | 239 |
| 115 | VIII-33 | 624 | 240 |
| 116 | VIII-41 | 649 | 245 |
| 117 | VIII-42 | 600 | 246 |
| 118 | VIII-46 | 425 | 249 |
| 119 | VIII-48 | 251 | 251 |
| 120 | VIII-64 | 627 | 261 |
| 121 | VIII-66 | 345 | 262 |
| 122 | VIII-67 | 252 | 263 |
| 123 | VIII-76 | 591 | 270 |
| 124 | X-07 | 641 | 328 |
| 125 | X-15 | 132 | 329 |
| 126 | X-29 | 370 | 331 |
| 127 | X-54 | 603 | 334 |
| 128 | X-56 | 71 | 335 |
| 129 | X-68 | 642 | 421 |
| 130 | X-72 | 622 | 336 |
| 131 | X-94 | 501 | 337 |
| 132 | XI-13 | 620 | 423 |
| 133 | XI-81 | 374 | 426 |
| 134 | XII-07 | 567 | 427 |
| 135 | XII-35 | 620 | 428 |
| 136 | XII-59 | 484 | 430 |
| 137 | XIII-19 | 559 | 433 |
| 138 | XIII-52 | 513 | 378 |
| 139 | XIII-92 | 741 | 435 |
| 140 | XV-22 | 561 | 388 |
| 141 | XV-25 | 485 | 436 |
| 142 | XVI-36 | 435 | 382 |
| 143 | XVI-53 | 741 | 439 |
| 144 | XVI-66 | 689 | 384 |
| 145 | XVI-76 | 198 | 386 |
| 146 | XVI-77 | 198 | 387 |
| 147 | XVII-31 | 503 | 392 |
| 148 | XVII-40 | 203 | 440 |
| 149 | XVII-48 | 587 | 393 |
| 150 | XVII-76 | 650 | 394 |
| 151 | XVII-87 | 502 | 395 |
| 152 | XVII-95 | 648 | 396 |

TABLE 1b
List of sequences of probes informative for disease diagnosis

| | SEQ ID NO. in |
|---|---|
| Clone ID | Sequence Listing |
| I-10 | 6 |
| I-13 | 444 |
| I-14 | 397 |
| I-15 | 7 |
| I-17 | 8 |
| I-19 | 9 |
| I-22 | 10 |
| I-24 | 11 |
| I-25 | 12 |
| I-28 | 13 |
| I-30 | 398 |
| I-31 | 14 |
| I-34 | 15 |
| I-37 | 482 |
| I-38 | 16 |
| I-39 | 17 |
| I-40 | 18 |
| I-42 | 445 |
| I-48 | 19 |
| I-49 | 20 |
| I-53 | 21 |
| I-54 | 399 |
| I-56 | 22 |
| I-57 | 23 |
| I-58 | 24 |
| I-60 | 25 |
| I-64 | 26 |
| I-67 | 27 |
| I-69 | 28 |
| I-77 | 29 |
| I-80 | 30 |
| I-81 | 31 |
| I-82 | 32 |
| I-86 | 447 |
| I-88 | 400 |
| I-95 | 448 |
| II-02 | 33 |
| II-03 | 34 |
| II-05 | 35 |
| II-06 | 36 |
| II-07 | 37 |
| II-08 | 38 |
| II-10 | 39 |
| II-11 | 40 |
| II-12 | 41 |
| II-13 | 42 |
| II-15 | 43 |
| II-16 | 44 |
| II-21 | 45 |
| II-23 | 46 |
| II-24 | 47 |
| II-25 | 48 |
| II-26 | 49 |
| II-27 | 50 |
| II-29 | 51 |
| II-30 | 52 |
| II-31 | 53 |
| II-32 | 54 |
| II-33 | 55 |
| II-34 | 56 |
| II-38 | 57 |
| II-39 | 58 |
| II-40 | 59 |
| II-41 | 60 |
| II-42 | 61 |
| II-43 | 62 |
| II-44 | 63 |
| II-46 | 64 |
| II-47 | 65 |
| II-48 | 66 |
| II-50 | 67 |
| II-52 | 68 |
| II-53 | 69 |
| II-54 | 70 |
| II-55 | 71 |
| II-56 | 72 |
| II-57 | 73 |
| II-58 | 74 |
| II-59 | 75 |
| II-60 | 76 |
| II-61 | 77 |
| II-62 | 78 |
| II-63 | 79 |
| II-64 | 80 |
| II-65 | 81 |
| II-66 | 82 |
| II-67 | 83 |
| II-68 | 84 |
| II-69 | 85 |
| II-70 | 86 |
| II-71 | 87 |
| II-72 | 88 |
| II-73 | 89 |
| II-74 | 90 |
| II-75 | 91 |
| II-76 | 92 |
| II-77 | 93 |
| II-78 | 94 |
| II-79 | 95 |
| II-80 | 96 |
| II-81 | 97 |
| II-82 | 98 |
| II-84 | 99 |
| II-87 | 100 |
| II-88 | 101 |
| II-92 | 102 |
| II-93 | 103 |
| II-94 | 104 |
| II-96 | 105 |
| III-01 | 106 |
| III-02 | 107 |
| III-03 | 108 |
| III-06 | 109 |
| III-07 | 110 |
| III-08 | 111 |
| III-09 | 112 |
| III-11 | 113 |
| III-12 | 114 |
| III-13 | 115 |
| III-18 | 116 |
| III-20 | 401 |
| III-21 | 117 |
| III-22 | 118 |
| III-23 | 119 |
| III-24 | 120 |
| III-25 | 121 |
| III-26 | 122 |
| III-27 | 123 |
| III-28 | 124 |
| III-29 | 125 |
| III-31 | 126 |
| III-32 | 127 |
| III-33 | 128 |
| III-34 | 129 |
| III-35 | 130 |
| III-39 | 131 |
| III-40 | 132 |
| III-42 | 133 |
| III-43 | 500 |
| III-44 | 134 |
| III-45 | 135 |
| III-46 | 136 |
| III-47 | 137 |
| III-48 | 138 |
| III-49 | 139 |
| III-50 | 140 |
| III-52 | 141 |
| III-53 | 142 |
| III-55 | 143 |
| III-56 | 144 |
| III-57 | 145 |
| III-58 | 146 |
| III-59 | 147 |
| III-61 | 148 |
| III-62 | 149 |
| III-63 | 150 |
| III-64 | 151 |
| III-66 | 152 |
| III-67 | 153 |
| III-70 | 154 |
| III-74 | 155 |
| III-76 | 156 |
| III-78 | 157 |
| III-80 | 158 |
| III-81 | 159 |
| III-82 | 451 |
| III-83 | 160 |
| III-85 | 161 |
| III-86 | 162 |
| III-88 | 163 & 164 |
| III-89 | 165 |
| III-92 | 452 |
| III-93 | 166 |
| III-94 | 167 |
| III-95 | 168 |
| IV-04 | 273 |
| IV-13 | 274 |
| IV-14 | 275 |
| IV-15 | 402 |
| IV-17 | 276 |
| IV-23 | 454 |
| IV-26 | 403 |
| IV-28 | 277 |
| IV-31 | 278 |
| IV-32 | 279 |
| IV-35 | 455 |
| IV-37 | 497 |
| IV-38 | 280 |
| IV-40 | 281 |
| IV-42 | 282 |
| IV-43 | 441 |
| IV-44 | 283 |
| IV-47 | 284 |
| IV-53 | 498 |
| IV-55 | 285 |
| IV-61 | 286 |
| IV-64 | 287 |
| IV-65 | 288 |
| IV-69 | 4 |
| IV-72 | 289 |
| IV-73 | 290 |
| IV-80 | 291 |
| IV-85 | 292 |
| IV-93 | 293 |
| TV-95 | 294 |
| IV-96 | 295 |
| IX-10 | 314 |
| IX-13 | 315 |
| IX-24 | 316 |
| IX-38 | 317 |
| IX-39 | 318 |
| IX-48 | 319 |
| IX-50 | 320 |
| IX-56 | 321 |
| IX-62 | 322 |
| IX-65 | 323 |
| IX-72 | 324 |
| IX-77 | 325 |
| IX-91 | 326 |
| IX-96 | 327 |
| V-01 | 458 |
| V-03 | 296 |
| V-04 | 297 |
| V-07 | 298 |
| V-08 | 299 |
| V-09 | 300 |
| V-11 | 404 |
| V1-16 | 344 |
| V1-19 | 345 |
| V-12 | 301 |
| V-17 | 459 |
| V-20 | 302 |
| V-24 | 303 |
| V-25 | 460 |
| V-28 | 405 |
| V-35 | 461 |
| V-38 | 406 |
| V-39 | 389 |
| V-40 | 304 |
| V-41 | 305 |
| V-47 | 463 |
| V-48 | 306 |
| V-49 | 464 |
| V-55 | 499 |
| V-57 | 307 |
| V-58 | 465 |
| V-61 | 308 |
| V-64 | 309 |
| V-68 | 484 |
| V-71 | 496 |
| V-74 | 310 |
| V-75 | 467 |
| V-80 | 311 |
| V-81 | 312 |
| V-87 | 313 |
| V-90 | 468 |
| VI-12 | 341 |
| VI-13 | 342 |
| VI-14 | 343 |
| VI-16 | 344 |
| VI-23 | 347 |
| VI-24 | 348 |
| VI-32 | 351 |
| VI-39 | 352 |
| VI-43 | 471 |
| VI-44 | 409 |
| VI-45 | 353 |
| VI-49 | 501 |
| VI-50 | 356 |
| VI-53 | 357 |
| VI-55 | 359 |
| VI-58 | 361 |
| VI-66 | 363 |
| VI-67 | 364 |
| VI-70 | 2 |
| VI-71 | 472 |
| VI-74 | 365 |
| VI-75 | 366 |
| VI-76 | 367 |
| VI-77 | 3 |
| VI-79 | 473 |
| VI-80 | 368 |
| VI-85 | 369 |
| VI-87 | 370 |
| VI-88 | 371 |
| VI-90 | 474 |
| VI-93 | 475 |
| VI-95 | 374 |
| VI-96 | 476 |
| VII-17 | 169 |
| VII-18 | 170 |
| VII-19 | 171 |
| VII-20 | 172 |
| VII-21 | 173 |
| VII-22 | 174 |
| VII-23 | 175 |
| VII-24 | 176 |
| VII-25 | 480 |
| VII-26 | 5 |
| VII-27 | 177 |
| VII-29 | 178 |
| VII-32 | 179 |
| VII-33 | 180 |
| VII-35 | 181 |
| VII-36 | 182 |
| VII-39 | 183 |
| VII-40 | 184 |
| VII-41 | 185 |
| VII-42 | 186 |
| VII-43 | 187 |
| VII-44 | 188 |
| VII-45 | 189 |
| VII-46 | 190 |
| VII-47 | 415 |
| VII-49 | 191 |
| VII-50 | 192 |
| VII-52 | 193 |
| VII-53 | 194 |
| VII-54 | 195 |
| VII-55 | 196 |
| VII-57 | 197 |
| VII-58 | 198 |
| VII-59 | 199 |
| VII-62 | 200 |
| VII-63 | 201 |
| VII-64 | 202 |
| VII-65 | 203 |
| VII-66 | 204 |
| VII-67 | 481 |
| VII-71 | 205 |
| VII-72 | 206 |
| VII-73 | 207 |
| VII-74 | 208 |
| VII-76 | 209 |
| VII-80 | 210 |
| VII-81 | 211 |
| VII-82 | 212 |
| VII-84 | 213 |
| VII-86 | 487 |
| VII-87 | 214 |
| VII-89 | 215 |
| VII-90 | 216 |
| VII-91 | 217 |
| VII-92 | 218 |
| VII-93 | 219 |
| VII-96 | 220 |
| VIII-09 | 221 |
| VIII-10 | 222 |
| VIII-12 | 223 |
| VIII-13 | 224 |
| VIII-16 | 225 |
| VIII-17 | 226 |
| VIII-18 | 227 |
| VIII-19 | 228 |
| VIII-20 | 229 |
| VIII-21 | 230 |
| VIII-23 | 231 |
| VIII-24 | 232 |
| VIII-25 | 233 |
| VIII-26 | 489 |
| VIII-27 | 234 |
| VIII-28 | 235 |
| VIII-29 | 236 |
| VIII-30 | 237 |
| VIII-31 | 238 |
| VIII-32 | 239 |
| VIII-33 | 240 |
| VIII-36 | 241 |
| VIII-37 | 242 |
| VIII-38 | 243 |
| VIII-40 | 244 |
| VIII-41 | 245 |
| VIII-42 | 246 |
| VIII-43 | 247 |
| VIII-45 | 248 |
| VIII-46 | 249 |
| VIII-47 | 250 |
| VIII-48 | 251 |
| VIII-50 | 252 |
| VIII-51 | 253 |
| VIII-53 | 254 |
| VIII-54 | 255 |
| VIII-55 | 256 |
| VIII-56 | 257 |
| VIII-57 | 258 |
| VIII-59 | 259 |
| VIII-60 | 260 |
| VIII-64 | 261 |
| VIII-66 | 262 |
| VIII-67 | 263 |
| VIII-70 | 264 |
| VIII-71 | 265 |
| VIII-72 | 266 |
| VIII-73 | 267 |
| VIII-74 | 268 |
| VIII-75 | 269 |
| VIII-76 | 270 |
| VIII-77 | 271 |
| VIII-80 | 272 |
| X-07 | 328 |
| X-15 | 329 |
| X-20 | 330 |
| X-29 | 331 |
| X-34 | 332 |
| X-46 | 333 |
| X-54 | 334 |
| X-56 | 335 |
| X-68 | 421 |
| X-72 | 336 |
| X-73 | 422 |
| X-94 | 337 |
| XI-13 | 423 |
| XI-37 | 490 |
| XI-43 | 424 |
| XI-67 | 425 |
| XI-81 | 426 |
| XII-07 | 427 |
| XII-35 | 428 |
| XII-36 | 429 |
| XII-59 | 430 |
| XII-65 | 381 |
| XII-92 | 431 |
| XIII-03 | 375 |
| XIII-04 | 432 |
| XIII-19 | 433 |
| XIII-24 | 376 |
| XIII-51 | 377 |
| XIII-52 | 378 |
| XIII-67 | 379 |
| XIII-69 | 380 |
| XIII-88 | 434 |
| XIII-92 | 435 |
| XV-22 | 388 |
| XV-25 | 436 |
| XV-62 | 437 |
| XV-64 | 390 |
| XV-84 | 391 |
| XVI-19 | 438 |
| XVI-36 | 382 |
| XVI-53 | 439 |
| XVI-60 | 383 |
| XVI-66 | 384 |
| XVI-74 | 385 |
| XVI-76 | 386 |
| XVI-77 | 387 |
| XVII-31 | 392 |
| XVII-40 | 440 |
| XVII-48 | 393 |
| XVII-76 | 394 |
| XVII-87 | 395 |
| XVII-95 | 396 |

TABLE 2a
List of informative probes for diagnosis of breast cancer

| Clone ID | |
|---|---|
| | SEQ ID NO. |
| | in |
| | Sequence |
| | Listing |
| I-24 | 11 |
| I-28 | 13 |
| I-30 | 398 |
| I-54 | 399 |
| II-41 | 60 |
| II-70 | 86 |
| II-87 | 100 |
| III-06 | 109 |
| III-20 | 401 |
| III-40 | 132 |
| III-57 | 145 |
| III-61 | 148 |
| III-89 | 165 |
| IV-14 | 275 |
| IV-15 | 402 |
| IV-26 | 403 |
| IV-32 | 279 |
| IV-53 | 498 |
| IV-69 | 4 |
| IV-80 | 291 |
| IX-10 | 314 |
| IX-38 | 317 |
| IX-48 | 319 |
| IX-77 | 325 |
| V-11 | 404 |
| V-55 | 499 |
| V-80 | 311 |
| VI-07 | 1 |
| VI-48 | 355 |
| VI-55 | 359 |
| VI-70 | 2 |
| VII-03 | 411 |
| VII-15 | 414 |
| VII-32 | 179 |
| VII-39 | 183 |
| VII-47 | 415 |
| VII-48 | 416 |
| VII-73 | 207 |
| VII-77 | 418 |
| VII-90 | 216 |
| VIII-20 | 229 |
| VIII-29 | 236 |
| VIII-30 | 237 |
| VIII-31 | 238 |
| VIII-46 | 249 |
| VIII-48 | 251 |
| VIII-66 | 262 |
| VIII-76 | 270 |
| X-07 | 328 |
| X-15 | 329 |
| X-29 | 331 |
| X-54 | 334 |
| X-56 | 335 |
| X-68 | 421 |
| X-72 | 336 |
| X-94 | 337 |
| XI-13 | 423 |
| XI-81 | 426 |
| XII-07 | 427 |
| XII-35 | 428 |
| | Sequence ID |
| XII-59 | 430 |
| XIII-19 | 433 |
| XIII-52 | 378 |
| XIII-92 | 435 |
| XV-22 | 388 |
| XV-25 | 436 |
| XVI-36 | 382 |
| XVI-53 | 439 |
| XVI-66 | 384 |
| XVI-76 | 386 |
| XVI-77 | 387 |
| XVII-31 | 392 |
| XVII-40 | 440 |
| XVII-48 | 393 |
| XVII-76 | 394 |
| XVII-87 | 395 |
| XVII-95 | 396 |

TABLE 2b
List of sequences of probes informative for breast cancer

| | SEQ ID NO. |
|---|---|
| | in Sequence |
| Clone ID | Listing |
| I-13 | 444 |
| I-14 | 397 |
| I-24 | 11 |
| I-25 | 12 |
| I-28 | 13 |
| I-30 | 398 |
| I-37 | 482 |
| I-42 | 445 |
| I-48 | 19 |
| I-54 | 399 |
| I-60 | 25 |
| I-72 | 446 |
| I-81 | 31 |
| I-82 | 32 |
| I-86 | 447 |
| I-88 | 400 |
| I-95 | 448 |
| II-02 | 33 |
| II-03 | 34 |
| II-06 | 36 |
| II-07 | 37 |
| II-10 | 39 |
| II-21 | 45 |
| II-23 | 46 |
| II-24 | 47 |
| II-25 | 48 |
| II-27 | 50 |
| II-33 | 55 |
| II-34 | 56 |
| II-41 | 60 |
| II-42 | 61 |
| II-46 | 64 |
| II-47 | 449 |
| II-48 | 66 |
| II-52 | 68 |
| II-57 | 73 |
| II-58 | 74 |
| II-59 | 75 |
| II-60 | 76 |
| II-61 | 77 |
| II-62 | 78 |
| II-64 | 80 |
| II-67 | 83 |
| II-69 | 85 |
| II-70 | 86 |
| II-74 | 90 |
| II-80 | 96 |
| II-82 | 98 |
| II-84 | 99 |
| II-87 | 100 |
| II-88 | 101 |
| II-96 | 105 |
| III-01 | 106 |
| III-02 | 107 |
| III-06 | 109 |
| III-08 | 111 |
| III-12 | 114 |
| III-13 | 115 |
| III-17 | 450 |
| III-18 | 116 |
| III-20 | 401 |
| III-21 | 117 |
| III-23 | 119 |
| III-24 | 120 |
| III-25 | 121 |
| III-26 | 122 |
| III-27 | 123 |
| III-28 | 124 |
| III-29 | 125 |
| III-32 | 127 |
| III-33 | 128 |
| III-35 | 130 |
| III-39 | 131 |
| III-40 | 132 |
| III-42 | 133 |
| III-45 | 135 |
| III-46 | 136 |
| III-47 | 137 |
| III-48 | 138 |
| III-56 | 144 |
| III-57 | 145 |
| III-58 | 146 |
| III-59 | 147 |
| III-61 | 148 |
| III-62 | 149 |
| III-63 | 150 |
| III-64 | 151 |
| III-66 | 152 |
| III-67 | 153 |
| III-70 | 154 |
| III-74 | 155 |
| III-75 | 156 |
| III-78 | 157 |
| III-80 | 158 |
| III-81 | 159 |
| III-82 | 451 |
| III-85 | 161 |
| III-86 | 162 |
| III-88 | 163 + 164 |
| III-89 | 165 |
| III-92 | 452 |
| III-93 | 166 |
| III-95 | 168 |
| III-96 | 452 |
| IV-04 | 273 |
| IV-13 | 274 |
| IV-14 | 275 |
| IV-15 | 402 |
| IV-17 | 276 |
| IV-23 | 454 |
| IV-26 | 403 |
| IV-31 | 278 |
| IV-32 | 279 |
| IV-35 | 455 |
| IV-37 | 497 |
| IV-38 | 280 |
| IV-42 | 282 |
| IV-43 | 441 |
| IV-47 | 284 |
| IV-53 | 498 |
| IV-61 | 286 |
| IV-64 | 287 |
| IV-69 | 4 |
| IV-72 | 289 |
| IV-80 | 291 |
| IV-85 | 292 |
| IV-93 | 457 |
| IV-96 | 295 |
| IX-10 | 314 |
| IX-13 | 315 |
| IX-24 | 316 |
| IX-38 | 317 |
| IX-39 | 318 |
| IX-48 | 319 |
| IX-50 | 320 |
| IX-56 | 321 |
| IX-62 | 322 |
| IX-65 | 323 |
| IX-72 | 324 |
| IX-77 | 325 |
| IX-91 | 326 |
| IX-96 | 327 |
| V-01 | 458 |
| V-03 | 296 |
| V-04 | 297 |
| V-07 | 298 |
| V-08 | 299 |
| V-11 | 404 |
| V-12 | 301 |
| V-17 | 459 |
| V-24 | 303 |
| V-25 | 460 |
| V-28 | 405 |
| V-38 | 461 |
| V-38 | 406 |
| V-39 | 389 |
| V-41 | 305 |
| V-47 | 463 |
| V-49 | 464 |
| V-55 | 499 |
| V-57 | 307 |
| V-58 | 465 |
| V-61 | 308 |
| V-64 | 309 |
| V-65 | 466 |
| V-68 | 484 |
| V-71 | 496 |
| V-74 | 310 |
| V-75 | 467 |
| V-80 | 311 |
| V-90 | 468 |
| VI-03 | 338 |
| VI-04 | 339 |
| VI-07 | 1 |
| VI-08 | 340 |
| VI-09 | 469 |
| VI-12 | 341 |
| VI-13 | 342 |
| VI-14 | 343 |
| VI-16 | 344 |
| VI-19 | 345 |
| VI-20 | 346 |
| VI-21 | 470 |
| VI-23 | 347 |
| VI-24 | 348 |
| VI-25 | 408 |
| VI-26 | 349 |
| VI-32 | 351 |
| VI-39 | 352 |
| VI-43 | 471 |
| VI-44 | 409 |
| VI-45 | 353 |
| VI-48 | 355 |
| VI-49 | 501 |
| VI-50 | 356 |
| VI-53 | 357 |
| VI-55 | 359 |
| VI-58 | 361 |
| VI-66 | 363 |
| VI-67 | 364 |
| VI-70 | 2 |
| VI-71 | 472 |
| VI-74 | 365 |
| VI-75 | 366 |
| VI-76 | 367 |
| VI-77 | 3 |
| VI-79 | 473 |
| VI-80 | 368 |
| VI-85 | 369 |
| VI-87 | 370 |
| VI-88 | 371 |
| VI-90 | 474 |
| VI-93 | 475 |
| VI-95 | 374 |
| VI-96 | 476 |
| VII-02 | 410 |
| VII-03 | 411 |
| VII-06 | 477 |
| VII-08 | 412 |
| VII-09 | 413 |
| VII-10 | 478 |
| VII-11 | 479 |
| VII-15 | 414 |
| VII-17 | 169 |
| VII-19 | 171 |
| VII-21 | 173 |
| VII-22 | 174 |
| VII-23 | 175 |
| VII-24 | 176 |
| VII-25 | 480 |
| VII-26 | 5 |
| VII-27 | 177 |
| VII-29 | 178 |
| VII-32 | 179 |
| VII-33 | 180 |
| VII-36 | 182 |
| VII-39 | 183 |
| VII-41 | 185 |
| VII-42 | 186 |
| VII-43 | 187 |
| VII-46 | 190 |
| VII-47 | 415 |
| VII-48 | 416 |
| VII-49 | 191 |
| VII-54 | 195 |
| VII-57 | 197 |
| VII-58 | 198 |
| VII-59 | 199 |
| VII-62 | 200 |
| VII-63 | 417 |
| VII-64 | 202 |
| VII-66 | 204 |
| VII-67 | 481 |
| VII-72 | 206 |
| VII-73 | 207 |
| VII-77 | 418 |
| VII-80 | 210 |
| VII-82 | 212 |
| VII-86 | 487 |
| VII-87 | 214 |
| VII-90 | 216 |
| VII-91 | 217 |
| VII-92 | 218 |
| VII-93 | 219 |
| VII-96 | 220 |
| VIII-09 | 221 |
| VIII-10 | 222 |
| VIII-13 | 224 |
| VIII-16 | 225 |
| VIII-20 | 229 |
| VIII-21 | 230 |
| VIII-23 | 231 |
| VIII-24 | 232 |
| VIII-25 | 233 |
| VIII-26 | 489 |
| VIII-27 | 234 |
| VIII-28 | 235 |
| VIII-29 | 236 |
| VIII-30 | 237 |
| VIII-31 | 238 |
| VIII-32 | 239 |
| VIII-33 | 240 |
| VIII-34 | 419 |
| VIII-38 | 243 |
| VIII-40 | 244 |
| VIII-41 | 245 |
| VIII-46 | 249 |
| VIII-48 | 251 |
| VIII-55 | 256 |
| VIII-57 | 258 |
| VIII-59 | 259 |
| VIII-60 | 260 |
| VIII-61 | 420 |
| VIII-64 | 261 |
| VIII-66 | 262 |
| VIII-73 | 267 |
| VIII-74 | 268 |
| VIII-76 | 270 |
| VIII-80 | 272 |
| X-07 | 328 |
| X-15 | 329 |
| X-20 | 330 |
| X-29 | 331 |
| X-34 | 332 |
| X-46 | 333 |
| X-54 | 334 |
| X-56 | 335 |
| X-68 | 421 |
| X-72 | 336 |
| X-73 | 422 |
| X-94 | 337 |
| XI-13 | 423 |
| XI-37 | 490 |
| XI-43 | 424 |
| XI-67 | 425 |
| XI-81 | 426 |
| XII-07 | 427 |
| XII-35 | 428 |
| XII-36 | 429 |
| XII-59 | 430 |
| XII-65 | 381 |
| XII-92 | 431 |
| XIII-03 | 375 |
| XIII-04 | 432 |
| XIII-19 | 433 |
| XIII-24 | 376 |
| XIII-51 | 377 |
| XIII-52 | 378 |
| XIII-67 | 379 |
| XIII-69 | 380 |
| XIII-88 | 434 |
| XIII-92 | 435 |
| XV-22 | 388 |
| XV-25 | 436 |
| XV-62 | 437 |
| XV-64 | 390 |
| XV-84 | 391 |
| XVI-19 | 438 |
| XVI-36 | 382 |
| XVI-53 | 439 |
| XVI-60 | 383 |
| XVI-66 | 384 |
| XVI-74 | 385 |
| XVI-76 | 386 |
| XVI-77 | 387 |
| XVII-31 | 392 |
| XVII-40 | 440 |
| XVII-48 | 393 |
| XVII-76 | 394 |
| XVII-87 | 395 |
| XVII-95 | 396 |

TABLE 3
List of informative probes (Clone ID) selected for breast cancer
diagnosis based on their occurrence criterion during variable selection

| Occurrence* | Clone ID |
|---|---|
| 100% | XI-8, XVI-66, VIII-66, XVI-59, VII-03, XIII-19, |
| | XII-35, X-35, XI-50, XII-26, IV-53, XIII-29, |
| | XIII-62, I-30, III-06,XV-22, XV-94, VII-15, |
| | VII-39, IX-39, XVII-39, III-40, VII-32 |
| 90% | I-52, VI-65, VI-34, IV-62, XV-34, XVII-58, |
| | V-11, VI-78, XII-36, XIII-92, VIII-29, XVI-53, |
| | XVI-77, XI-13, XIII-84, IV-14, XII-31, V-80, |
| | VII-48, XVII-29, XVII-72 |
| 80% | III-60, VIII-74, IX-12, X-04, XIII-52, VIII-30, |
| | IX-38 |
| 70% | VI-49, X-29, VIII-48 |
| 60% | IV-82, IX-10, VI-52, X-68, VII-77 |
| 50% | IV-15 |
| 40% | XV-28, II-70, V-55 |
| 30% | XVII-17, XVII-67 |
| 20% | XI-58, XVI-36, VIII-39, VIII-44, III-61, IV-69, |
| | XV-68, X-72 |
| 10% | IX-42, IX-77, X-94, XV-96, XVII-55 |
| 5% | XII-59, XVI-76, I-54, XV-18, V-94, X-54, VI-07, |
| | VII-47, XVII-31, XVII-87, XVII-48 |
| In at least one model | II-41, VI-41, III-57, III-89, VII-73, XV-25, |
| | IV-26, X-34, IV-41, VII-90, XV-42, XVII-82, |
| | XII-27, VIII-20, I-28, VII-60, VIII-76, III-20, |
| | VI-84, XI-07, XVII-28, XII-17, XVII-36, |
| | XII-52, XVII-76, VIII-46, VI-70, XV-74, |
| | XV-93, VIII-31, II-87, V-39, VI-55, |
| | X-07, X-15, XII-07, XVII-07, XVII-08, |
| | XVII-95, I-24, IV-32, V-32, VI-48, VI-72, |
| | IV-80, IX-48, X-56, XV-24, XII-32, |
| | XVII-40 |

*100% = Genes appearing in all the 75 cross validated models; 90% = Additional genes appearing in at least 68 out of 75 cross validated models; 5% = Additional genes appearing in at least 4 out of 75 cross validated models and so on.

TABLE 4a
List of informative probes for diagnosis of Alzheimer disease

| | SEQ ID NO. |
|---|---|
| | in |
| Clone | Sequence |
| ID | Listing |
| I-34 | 15 |
| I-58 | 24 |
| II-03 | 34 |
| II-05 | 35 |
| II-06 | 36 |
| II-10 | 39 |
| II-24 | 47 |
| II-25 | 48 |
| II-26 | 49 |
| II-33 | 55 |
| II-34 | 56 |
| II-42 | 61 |
| II-57 | 73 |
| II-61 | 77 |
| II-69 | 85 |
| II-75 | 91 |
| II-84 | 99 |
| II-88 | 101 |
| II-94 | 104 |
| III-02 | 107 |
| III-06 | 109 |
| III-08 | 111 |
| III-13 | 115 |
| III-23 | 119 |
| III-26 | 122 |
| III-35 | 130 |
| III-39 | 131 |
| III-43 | 500 |
| III-44 | 134 |
| III-53 | 142 |
| III-56 | 144 |
| III-63 | 150 |
| III-74 | 155 |
| III-80 | 158 |
| III-85 | 161 |
| IV-31 | 278 |
| IV-80 | 291 |
| V-03 | 296 |
| V-04 | 297 |
| V-07 | 298 |
| V-12 | 301 |
| V-80 | 311 |
| VI-04 | 339 |
| VI-12 | 341 |
| VI-14 | 343 |
| VI-20 | 346 |
| VI-23 | 347 |
| VI-48 | 355 |
| VI-50 | 356 |
| VI-53 | 357 |
| VI-74 | 365 |
| VI-76 | 367 |
| VI-87 | 370 |
| VI-88 | 371 |
| VI-95 | 374 |
| VII-19 | 171 |
| VII-21 | 173 |
| VII-36 | 182 |
| VII-42 | 186 |
| VII-43 | 187 |
| VII-46 | 190 |
| VII-59 | 199 |
| VII-63 | 201 |
| VII-66 | 204 |
| VII-72 | 206 |
| VII-73 | 207 |
| VI-12 | 344 |
| VI-14 | 345 |
| VII-91 | 217 |
| VII-93 | 219 |
| VIII-09 | 221 |
| VIII-28 | 235 |
| VIII-30 | 237 |
| VIII-32 | 239 |
| VIII-33 | 240 |
| VIII-41 | 245 |
| VIII-42 | 246 |
| VIII-48 | 251 |
| VIII-64 | 261 |
| VIII-67 | 263 |

TABLE 4b
List of sequences of probes informative for Alzheimer dis

| | SEQ ID NO. in |
|---|---|
| Clone ID | Sequence Listing |
| I-10 | 6 |
| I-15 | 7 |
| I-17 | 8 |
| I-19 | 9 |
| I-22 | 10 |
| I-24 | 11 |
| I-25 | 12 |
| I-28 | 13 |
| I-31 | 14 |
| I-34 | 15 |
| I-38 | 16 |
| I-39 | 17 |
| I-40 | 18 |
| I-48 | 19 |
| I-49 | 20 |
| I-53 | 21 |
| I-56 | 22 |
| I-57 | 23 |
| I-58 | 24 |
| I-60 | 25 |
| I-64 | 26 |
| I-67 | 27 |
| I-69 | 28 |
| I-77 | 29 |
| I-80 | 30 |
| I-81 | 31 |
| I-82 | 32 |
| II-02 | 33 |
| II-03 | 34 |
| II-05 | 35 |
| II-06 | 36 |
| II-07 | 37 |
| II-08 | 38 |
| II-10 | 39 |
| II-11 | 40 |
| II-12 | 41 |
| II-13 | 42 |
| II-15 | 43 |
| II-16 | 44 |
| II-21 | 45 |
| II-23 | 46 |
| II-24 | 47 |
| II-25 | 48 |
| II-26 | 49 |
| II-27 | 50 |
| II-29 | 51 |
| II-30 | 52 |
| II-31 | 53 |
| II-32 | 54 |
| II-33 | 55 |
| II-34 | 56 |
| II-38 | 57 |
| II-39 | 58 |
| II-40 | 59 |
| II-41 | 60 |
| II-42 | 61 |
| II-43 | 62 |
| II-44 | 63 |
| II-46 | 64 |
| II-47 | 65 |
| II-48 | 66 |
| II-50 | 67 |
| II-52 | 68 |
| II-53 | 69 |
| II-54 | 70 |
| II-55 | 71 |
| II-56 | 72 |
| II-57 | 73 |
| II-58 | 74 |
| II-59 | 75 |
| II-60 | 76 |
| II-61 | 77 |
| II-62 | 78 |
| II-63 | 79 |
| II-64 | 80 |
| II-65 | 81 |
| II-66 | 82 |
| II-67 | 83 |
| II-68 | 84 |
| II-69 | 85 |
| II-70 | 86 |
| II-71 | 87 |
| II-72 | 88 |
| II-73 | 89 |
| II-74 | 90 |
| II-75 | 91 |
| II-76 | 92 |
| II-77 | 93 |
| II-78 | 94 |
| II-79 | 95 |
| II-80 | 96 |
| II-81 | 97 |
| II-82 | 98 |
| II-84 | 99 |
| II-87 | 100 |
| II-88 | 101 |
| II-92 | 102 |
| II-93 | 103 |
| II-94 | 104 |
| II-96 | 105 |
| III-01 | 106 |
| III-02 | 107 |
| III-03 | 108 |
| III-06 | 109 |
| III-07 | 110 |
| III-08 | 111 |
| III-09 | 112 |
| III-11 | 113 |
| III-12 | 114 |
| III-13 | 115 |
| III-21 | 117 |
| III-22 | 118 |
| III-23 | 119 |
| III-24 | 120 |
| III-25 | 121 |
| III-26 | 122 |
| III-27 | 123 |
| III-28 | 124 |
| III-29 | 125 |
| III-31 | 126 |
| III-32 | 127 |
| III-33 | 128 |
| III-34 | 129 |
| III-35 | 130 |
| III-39 | 131 |
| III-40 | 132 |
| III-42 | 133 |
| III-43 | 500 |
| III-44 | 134 |
| III-45 | 135 |
| III-46 | 136 |
| III-47 | 137 |
| III-48 | 138 |
| III-49 | 139 |
| III-50 | 140 |
| III-52 | 141 |
| III-53 | 142 |
| III-55 | 143 |
| III-56 | 144 |
| III-57 | 145 |
| III-58 | 146 |
| III-59 | 147 |
| III-61 | 148 |
| III-62 | 149 |
| III-63 | 150 |
| III-64 | 151 |
| III-66 | 152 |
| III-67 | 153 |
| III-70 | 154 |
| III-74 | 155 |
| III-75 | 156 |
| III-78 | 157 |
| III-80 | 158 |
| III-81 | 159 |
| III-83 | 160 |
| III-85 | 161 |
| III-86 | 152 |
| III-88 | 163/164 |
| III-89 | 165 |
| III-93 | 166 |
| III-94 | 167 |
| III-95 | 168 |
| VII-17 | 169 |
| VII-18 | 170 |
| VII-19 | 171 |
| VII-20 | 172 |
| VII-21 | 173 |
| VII-22 | 174 |
| VII-23 | 175 |
| VII-24 | 176 |
| VII-27 | 177 |
| VII-29 | 178 |
| VII-32 | 179 |
| VII-33 | 180 |
| VII-35 | 181 |
| VII-36 | 182 |
| VII-39 | 183 |
| VII-40 | 184 |
| VII-41 | 185 |
| VII-42 | 186 |
| VII-43 | 187 |
| VII-44 | 188 |
| VII-45 | 189 |
| VII-46 | 190 |
| VII-49 | 191 |
| VII-50 | 192 |
| VII-52 | 193 |
| VII-53 | 194 |
| VII-54 | 195 |
| VII-55 | 196 |
| VII-57 | 197 |
| VII-58 | 198 |
| VII-59 | 199 |
| VII-62 | 200 |
| VII-63 | 201 |
| VII-64 | 202 |
| VII-65 | 203 |
| VII-66 | 204 |
| VII-71 | 205 |
| VII-72 | 206 |
| VII-73 | 207 |
| VII-74 | 208 |
| VII-76 | 209 |
| VII-80 | 210 |
| VII-81 | 211 |
| VII-82 | 212 |
| VII-84 | 213 |
| VII-87 | 214 |
| VII-89 | 215 |
| VII-90 | 216 |
| VII-91 | 217 |
| VII-92 | 218 |
| VII-93 | 219 |
| VII-96 | 220 |
| VIII-09 | 221 |
| VIII-10 | 222 |
| VIII-12 | 223 |
| VIII-13 | 224 |
| VIII-16 | 225 |
| VIII-17 | 226 |
| VIII-18 | 227 |
| VIII-19 | 228 |
| VIII-20 | 229 |
| VIII-21 | 230 |
| VIII-23 | 231 |
| VIII-24 | 232 |
| VIII-25 | 233 |
| VIII-28 | 235 |
| VIII-29 | 236 |
| VIII-30 | 237 |
| VIII-31 | 238 |
| VIII-32 | 239 |
| VIII-33 | 240 |
| VIII-36 | 241 |
| VIII-37 | 242 |
| VIII-38 | 243 |
| VIII-40 | 244 |
| VIII-41 | 245 |
| VIII-42 | 246 |
| VIII-43 | 247 |
| VIII-45 | 248 |
| VIII-46 | 249 |
| VIII-47 | 250 |
| VIII-48 | 251 |
| VIII-50 | 252 |
| VIII-51 | 253 |
| VIII-53 | 254 |
| VIII-54 | 255 |
| VIII-55 | 256 |
| VIII-56 | 257 |
| VIII-57 | 258 |
| VIII-59 | 259 |
| VIII-60 | 260 |
| VIII-64 | 261 |
| VIII-66 | 262 |
| VIII-67 | 263 |
| VIII-70 | 264 |
| VIII-71 | 265 |
| VIII-72 | 266 |
| VIII-73 | 267 |
| VIII-74 | 268 |
| VIII-75 | 269 |
| VIII-76 | 270 |
| VIII-77 | 271 |
| VIII-80 | 272 |
| IV-04 | 273 |
| IV-13 | 274 |
| IV-14 | 275 |
| IV-17 | 276 |
| IV-28 | 277 |
| IV-31 | 278 |
| IV-32 | 279 |
| IV-38 | 280 |
| IV-40 | 281 |
| IV-42 | 282 |
| IV-44 | 283 |
| IV-47 | 284 |
| IV-55 | 285 |
| IV-61 | 286 |
| IV-64 | 287 |
| IV-65 | 288 |
| IV-72 | 289 |
| IV-73 | 290 |
| IV-80 | 291 |
| IV-85 | 292 |
| IV-93 | 293 |
| IV-95 | 294 |
| IV-96 | 295 |
| V-03 | 296 |
| V-04 | 297 |
| V-07 | 298 |
| V-08 | 299 |
| V-09 | 300 |
| V-12 | 301 |
| V-20 | 302 |
| V-24 | 303 |
| V-40 | 304 |
| V-41 | 305 |
| V-48 | 306 |
| V-57 | 307 |
| V-61 | 308 |
| V-64 | 309 |
| V-74 | 310 |
| V-80 | 311 |
| V-81 | 312 |
| V-87 | 313 |
| VI-13 | 342 |
| VI-14 | 343 |
| VI-16 | 344 |
| VI-23 | 347 |
| VI-24 | 348 |
| VI-28 | 350 |
| VI-32 | 351 |
| VI-39 | 352 |
| VI-45 | 353 |
| VI-46 | 354 |
| VI-49 | 501 |
| VI-50 | 356 |
| VI-53 | 357 |
| VI-54 | 358 |
| VI-55 | 359 |
| VI-57 | 360 |
| VI-58 | 361 |
| VI-63 | 362 |
| VI-66 | 363 |
| VI-67 | 364 |
| VI-74 | 365 |
| VI-75 | 366 |
| VI-76 | 367 |
| VI-80 | 368 |
| VI-85 | 369 |
| VI-87 | 370 |
| VI-88 | 371 |
| VI-91 | 372 |
| VI-94 | 373 |
| VI-95 | 374 |
| I-14 | 397 |
| I-30 | 398 |
| I-54 | 399 |
| I-88 | 400 |
| III-20 | 401 |
| IV-15 | 402 |
| IV-26 | 403 |
| V-11 | 404 |
| IV-28 | 405 |
| IV-38 | 406 |
| IV-45 | 407 |
| VI-44 | 409 |
| VII-47 | 415 |
| I-42 | 445 |
| I-86 | 447 |
| I-95 | 448 |
| III-82 | 451 |
| III-92 | 452 |
| IV-23 | 454 |
| IV-35 | 455 |
| IV-82 | 456 |
| V-01 | 458 |
| V-17 | 459 |
| V-25 | 460 |
| V-35 | 461 |
| V-42 | 462 |
| V-47 | 463 |
| V-49 | 464 |
| V-58 | 465 |
| V-75 | 467 |
| V-90 | 468 |
| VI-43 | 471 |
| VI-71 | 472 |
| VI-79 | 473 |
| VI-90 | 474 |
| VI-93 | 475 |
| VII-25 | 480 |
| VII-67 | 481 |
| I-37 | 482 |
| V-52 | 483 |
| V-68 | 484 |
| V-92 | 485 |
| VI-42 | 486 |
| VII-86 | 487 |
| VII-88 | 488 |
| IV-29 | 491 |
| V-15 | 491 |
| V-39 | 493 |
| V-54 | 494 |
| V-59 | 495 |
| V-71 | 496 |

TABLE 5
Samples

| Diagnosis | No. of women |
|---|---|
| Normal/Benign | 42* |
| DCIS | 3 |
| Invasive cancer | 26 |

Information about women with breast cancer

| | | | | Size hist. | |
|---|---|---|---|---|---|
| Sample | AGE | Stage | Cancer type | (mm) | Nodes |
| 1 | 51 | II | IDC | 20 | 1/7 |
| 2 | 84 | II | IDC | 22 | 2/2 |
| 3 | 50 | I | DCIS + | >50 DCIS; | 0/7 |
| | | | 1 IDC | 5 × 14 | |
| 4 | 47 | I | IDC | 15 | 0 |
| 5 | 69 | III | ILC g.2 + tubular | 50 + 3 | 1 av 12 + 1 av 7 |
| | | | adenocarcinoma | | |
| 6 | 50 | II | IDC | 24 | 0 |
| 7 | 65 | I | IDC | 15 | 0 |
| 8 | 63 | II | IDC | 23 | 0 |
| 9 | 55 | I | IDC + DCIS | 4 | 0 av 1 |
| 10 | 52 | 0 | DCIS + small | 50 + 3 | 0 |
| | | | colloid carcinoma | | |
| | | | foci | | |
| 11 | 60 | II | IDC | 24 | 0 |
| 12 | 54 | I | IDC | 11 | 0 |
| 13 | | 0 | DCIS | 20 | 0 |
| 14 | 49 | 0 | DCIS | 9 | 0 |
| 15 | 48 | I | IDC | 4 | 0 |
| 16 | 56 | I | IDC | 4 | 0 |
| 17 | 68 | I | IDC | 14 | 0 |
| 18 | 68 | I | IDC | 7 | 0 |
| 19 | 63 | I | IDC | 10 | 0 |
| 20 | 45 | I | IDC | 19 | 1 |
| 21 | 57 | III | IDC | 60 | 8/20 |
| 22 | 55 | II | IDC/DCIS | 35 + 55 | 0 |
| 23 | 71 | I | IDC/extensive | 8 | 0 |
| | | | DCIS | | |
| 24 | 56 | I | IDC | 9 | ? |
| 25 | 66 | II | IDC | 26 | 0 |
| 26 | 66 | I | IDC | 15 | ? |
| 27 | 61 | I | IDC | 9 | ? |
| 28 | ? | ? | ? | ? | ? |
| 29 | 65 | I | IDC | 11 | 0 |

Other diseases/conditions present in the women tested
Other diseases/conditions present in the women tested

Disease/condition
Diabetes
Asthma
Ulcerous colitis
Hemochromatose
Crohn's disease
Fibromyalgia
Psoraiasis
Atopic eczema
Rheumatism
Allergies

Prior history of cancer in the women tested

| Cancer type | No. of women |
|---|---|
| Breast | 3 |
| Colon | 2 |
| Stomach | 1 |
| Skin | 1 |

*From one woman, whole blood was collected at weeks 1, 2, 3, 4, 5 following menstruation. Hence, the number of unique normal/benign samples tested in the experiment is 75.

TABLE 6
Number of samples tested by double cross validation and success of the
diagnostic test for breast cancer based on selected ionformative genes
Number of samples tested by double cross validation

| | |
|---|---|
| Number of unique samples tested | 75 |
| Number of unique non cancer samples tested | 46 |
| Number of cancer samples tested | 29 |

Success of the diagnostic test for breast cancer based on selected informative genes

| | Number of | | | | False | False | Total |
|---|---|---|---|---|---|---|---|
| Occurrence in | informative | | | | Positive | negative | error |
| percentage* | probes | Specificity | Sensitivity | Accuracy | rate | rate | rate |
| 100.00 | 23 | 84.78 | 75.86 | 81.33 | 15.22 | 24.14 | 18.67 |
| 90.00 | 44 | 91.30 | 79.31 | 86.67 | 8.70 | 20.69 | 13.33 |
| 80.00 | 51 | 86.96 | 79.31 | 84.00 | 13.04 | 20.69 | 16.00 |
| 70.00 | 54 | 89.13 | 75.86 | 84.00 | 10.87 | 24.14 | 16.00 |
| 60.00 | 58 | 89.13 | 75.86 | 84.00 | 10.87 | 24.14 | 16.00 |
| 50.00 | 59 | 89.13 | 75.86 | 84.00 | 10.87 | 24.14 | 16.00 |
| 40.00 | 63 | 89.13 | 75.86 | 84.00 | 10.87 | 24.14 | 16.00 |
| 30.00 | 66 | 86.96 | 75.86 | 82.67 | 13.04 | 24.14 | 17.33 |
| 20.00 | 74 | 89.13 | 75.86 | 84.00 | 10.87 | 24.14 | 16.00 |
| 10.00 | 79 | 89.13 | 75.86 | 84.00 | 10.87 | 24.14 | 16.00 |
| 5.00 | 90 | 86.96 | 79.31 | 84.00 | 13.04 | 20.69 | 16.00 |
| 1.33 | 139 | 84.78 | 72.41 | 80.00 | 15.22 | 27.59 | 20.00 |

*100% = Genes appearing in all the 75 cross validated models; 90% = Genes appearing in at least 68 out of 75 cross validated models; 5% = Genes appearing in at least 4 out of 75 cross validated models; and so on.

TABLE 7
Double cross-validation and details of the success of the diagnostic test
for Alzheimer disease based on the expression 182 informative genes
Validation Result

| | |
|---|---|
| Total number of samples | 14 |
| tested | |
| Number of Alzhelmer's | 7 |
| disease samples tested | |
| Number of Alzhelmer's | 1 |
| disease samples incorrectly | |
| predicted | |
| Number of non-Alzhelmer's | 7 |
| disease samples tested | |
| Number of non-Alzhelmer's | 0 |
| disease samples incorrectly | |
| predicted | |

Success of diagnostic test

| Performance | Description | % |
|---|---|---|
| Accuracy | Percentage of the total number of | 92.9 |
| | predictions that were correct | |
| Sensitivity | Percentage of positive cases that | 85.7 |
| | were correctly identified | |
| Specificity | Percentage of negatives cases | 100 |
| | that | |
| | were correctly predicted | |
| False positive | Percentage of negatives cases | 0.0 |
| rate | that | |
| | were incorrectly classified as | |
| | positive | |
| False negative | Percentage of positive cases that | 14.3 |
| rate | were incorrectly classified as | |
| | negative | |
| Total error rate | Percentage of the total cases | 7.1 |
| | incorrectly predicted | |

TABLE 8
Some relevant features of the blood donors.

| | | | Cancer type/ | | |
|---|---|---|---|---|---|
| | | | breast | Size Hist. | mRNA |
| | | AGE | abnormality | (mm) | Quality |
| 1 | B1 | na | IDC | 5 | ++ |
| 2 | B2 | 49 | DCIS | 8 | nd |
| 3 | B3 | 54 | IDC | 18 | ++ |
| 4 | B4 | 59 | IDC | 12 | + |
| 5 | B5 | 61 | DCIS + micro | 15 + 1.5 | ++ |
| | | | invasive cancer | | |
| 6 | B6 | 55 | IDC | 12 + 17 | nd |
| 7 | B6 | | IDC | 12 + 17 | nd |
| 8 | N1 | 45 | Fibroadenoma | — | nd |
| 9 | N2 | 52 | na | — | + |
| 10 | N3 | 55 | Cyst | — | ++ |
| 11 | N4 | 54 | na | — | ++ |
| 12 | N5 | 51 | Benign ductal | — | nd |
| | | | epithelium | | |
| 13 | N6 | 57 | Benign | — | nd |
| 14 | N7 | 50 | na | — | ++ |
| 15 | N8 | 52 | na | — | + |

B, Female donors with breast cancer;
N, Female donors with suspected mammogram but no breast cancer;
IDC, invasive ductal carcinoma;
DCIS, ductal carcinoma in situ;
na, not available
nd, not determined;
++, no degradation of mRNA and no ribosomal contamination in the sample,
+, no degradation of mRNA but ribosomal contamination in the sample.

TABLE 9
List of sequences of probes informative for both alzheimer
and breast cancer disease

| | SEQ ID NO. in |
|---|---|
| Clone ID | Sequence Listing |
| I-24 | 11 |
| I-25 | 12 |
| I-28 | 13 |
| I-48 | 19 |
| I-60 | 25 |
| I-81 | 31 |
| I-82 | 32 |
| II-02 | 33 |
| II-03 | 34 |
| II-06 | 36 |
| II-07 | 37 |
| II-10 | 39 |
| II-21 | 45 |
| II-23 | 46 |
| II-24 | 47 |
| II-25 | 48 |
| II-27 | 50 |
| II-33 | 55 |
| II-34 | 56 |
| II-41 | 60 |
| II-42 | 61 |
| II-46 | 64 |
| II-47 | 65 |
| II-48 | 66 |
| II-52 | 68 |
| II-57 | 73 |
| II-58 | 74 |
| II-59 | 75 |
| II-60 | 76 |
| II-61 | 77 |
| II-62 | 78 |
| II-64 | 80 |
| II-67 | 83 |
| II-69 | 85 |
| II-70 | 86 |
| II-74 | 90 |
| II-80 | 96 |
| II-82 | 98 |
| II-84 | 99 |
| II-87 | 100 |
| II-88 | 101 |
| II-96 | 105 |
| III-01 | 106 |
| III-02 | 107 |
| III-06 | 109 |
| III-08 | 111 |
| III-12 | 114 |
| III-13 | 115 |
| III-18 | 116 |
| III-21 | 117 |
| III-23 | 119 |
| III-24 | 120 |
| III-25 | 121 |
| III-26 | 122 |
| III-27 | 123 |
| III-28 | 124 |
| III-29 | 125 |
| III-32 | 127 |
| III-33 | 128 |
| III-35 | 130 |
| III-39 | 131 |
| III-40 | 132 |
| III-42 | 133 |
| III-45 | 135 |
| III-46 | 136 |
| III-47 | 137 |
| III-48 | 138 |
| III-56 | 144 |
| III-57 | 145 |
| III-58 | 146 |
| III-59 | 147 |
| III-61 | 148 |
| III-62 | 149 |
| III-63 | 150 |
| III-64 | 151 |
| III-66 | 152 |
| III-67 | 153 |
| III-70 | 154 |
| III-74 | 155 |
| III-5 | 156 |
| III-78 | 157 |
| III-80 | 158 |
| III-81 | 159 |
| III-85 | 161 |
| III-86 | 162 |
| III-88 | 163/164 |
| III-89 | 165 |
| III-93 | 166 |
| III-95 | 168 |
| IV-04 | 273 |
| IV-13 | 274 |
| IV-14 | 275 |
| IV-17 | 276 |
| IV-31 | 278 |
| IV-32 | 279 |
| IV-38 | 280 |
| IV-42 | 282 |
| IV-47 | 284 |
| IV-61 | 286 |
| IV-64 | 287 |
| IV-72 | 289 |
| IV-80 | 291 |
| IV-85 | 292 |
| IV-93 | 293 |
| IV-96 | 295 |
| V-03 | 296 |
| V-04 | 297 |
| V-07 | 298 |
| V-08 | 299 |
| V-12 | 301 |
| V-24 | 303 |
| V-41 | 305 |
| V-57 | 307 |
| V-61 | 308 |
| V-64 | 309 |
| V-74 | 310 |
| V-80 | 311 |
| VI-12 | 341 |
| VI-14 | 343 |
| VI-23 | 347 |
| VI-50 | 356 |
| VI-53 | 357 |
| VI-74 | 365 |
| VI-76 | 367 |
| VI-87 | 370 |
| VI-88 | 371 |
| VI-95 | 374 |
| VII-19 | 171 |
| VII-21 | 173 |
| VII-22 | 174 |
| VII-23 | 175 |
| VII-24 | 176 |
| VII-27 | 177 |
| VII-29 | 178 |
| VII-32 | 179 |
| VII-33 | 180 |
| VII-36 | 182 |
| VII-28 | 183 |
| VII-41 | 185 |
| VII-42 | 186 |
| VII-43 | 187 |
| VII-46 | 190 |
| VII-49 | 191 |
| VII-54 | 195 |
| VII-57 | 197 |
| VII-58 | 198 |
| VII-59 | 199 |
| VII-62 | 200 |
| VII-63 | 201 |
| VII-64 | 202 |
| VII-66 | 204 |
| VII-72 | 206 |
| VII-73 | 207 |
| VII-80 | 210 |
| VII-82 | 212 |
| VII-87 | 214 |
| VII-90 | 216 |
| VII-91 | 217 |
| VII-92 | 218 |
| VII-93 | 219 |
| VII-96 | 220 |
| VIII-09 | 221 |
| VIII-10 | 222 |
| VIII-13 | 224 |
| VIII-16 | 225 |
| VIII-20 | 229 |
| VIII-21 | 230 |
| VIII-23 | 231 |
| VIII-24 | 232 |
| VIII-25 | 233 |
| VIII-28 | 235 |
| VIII-29 | 236 |
| VIII-30 | 237 |
| VIII-31 | 238 |
| VIII-32 | 239 |
| VIII-33 | 240 |
| VIII-38 | 243 |
| VIII-40 | 244 |
| VIII-41 | 245 |
| VIII-46 | 249 |
| VIII-48 | 251 |
| VIII-55 | 256 |
| VIII-57 | 258 |
| VIII-59 | 259 |
| VIII-60 | 260 |
| VIII-64 | 261 |
| VIII-66 | 262 |
| VIII-73 | 267 |
| VIII-74 | 268 |
| VIII-76 | 270 |
| VIII-80 | 272 |

Nucleotide sequences
nt: 405
SEQ ID NO: 1
GGATCCTGTGGCCCACAGAGCTGCCCCAGCAGACGCTCCGCCCCACCCG
GTGATGGAGCCCCGGGGGGACAATCGTGCCTGGGGAGGAGCAGGGTACA
GCCCATTCCCCCAGCCCTGGCTGACCTGGCCTAGCAGTTTGGCCCTGCT
GGCCTTAGCAGGGAGACAGGGGAGCAAAGAACGCCAAGCCGGAGGCCCG
AGGCCAGCCGGCCTCTCGAGAGCCAGAGCAGCAGTTGAATGTAATGCTG
GGGACAGGCATGCTGCCGCCAGTAGGGCGGGGACCCGGACAGCCAGGTG
ACTACCAGTCCTGGGGACACACTCACCATAAACACATCCCCAGGCAGGA
CAGATCGGGGAAGGGGTGTGTACCAGGCTATGATTTCTCTTGCATTAAA
ATGTATTATTATT
nt: 550
SEQ ID NO: 2
GGCTTTGACAGAGTGCAAGACGATGACTTGCAAAATGTCGCATCTGGAA
CGCAACATAGANACCATCATCAACACCTTCCACCAATACTCTGTGAAGC
TGGGGCACCCAGACACCCTGAACCAGGGGGAATTCAAAGAGCTGGTGCG
AAAAGATCTGCAAAATTTTCTCAAGAAGGAGAATAAGAATGAAAAGGTC
ATAGAACACATCATGGAGGACCTGGACACAAATGCAGACAAGCAGCTGA
GCTTCGAGGAGTTCATCATGCTGATGGCGAGGCTAACCTGGGCCTCCCA
CGAGAAGATGCACGAGGGTGACGAGGGCCCTGGCCACCACCATAAGCCA
GGCCTCGGGGAGGGCACCCCCTAAGACCACAGTGGCCAAGATCACAGTG
GCCACGGCCACGGCCACAGTCATGGTGGCCACGGCCACAGCCACTAATC
AGGAGGCCAGGCCACCCTGCCTNTACCCAACCAGGGCCCCGGGGCCTGT
TATGTCAAACTGTCTTGGCTGTGGGGCTAGGGGCTGGGGCCAAATAAAG
TCTCTTTCTCC
SEQ ID NO: 3
ACGAAGACAGACATCTGTGGAATGATTCACATCCTCTCAAGTTAGGAGG
ATGGAGGCCTGCTTCATTAAGAAGCTGGGGGTAGGGTGGGGGTGGGGAG
AACACTTAACAACATGGGGACCAGTCAGGGGAATCCCCTTATTTCTGTT
TTGCATATGAGGAACCCTAGAGCAGCCAGGTGAGGCTCTCTAGTTTAAT
AAAAATCATGGAAAGACTCTTAATGCAGACTCTTCTTAAGTGTTAATAG
GGATTTTTTCAGCTTATTTTGGTTGCAGTTTCCAATTTTTAAAAATGTT
GAGGTAATCTTTCCCACCTTCCCAAACCTAATTCTTGTAGATGCATTAG
TGTTGAACCAATGCTTTCTCATGTCTCAATTCTTTGTATATGCATTCTT
TTCAGATGTATTAAACAAACAAAAACCCTTC
nt: 286
SEQ ID NO: 4
CCGGTAATAGAATAGAAAAGGGAGAGTGTCTTCATGCAATGTGGCATCC
TGGATTGGGTCTCGNNACAAAAACAGGACATTAGTGGGAAAATTGGAAA
TCTGAAAAAAGTCTGAATTTTAGTTAATATACCAATTTCAGTCTCTTGG
TTTTGACAGATGTACCATGGTGATGTAAGATGTTGACCTTGGGGTAGGC
TGGGTGAAGGGTATACAGGAACTCTTTGTACTATCTCTGCAACTTCTCT
GTAAATCTAGTATCATTCCAAAATAAAAGTTTATTTAATTT
SEQ ID NO: 5
GTGGAAGTGACATCGTCTTTAAACCCTGCGTGGCAATCCCTGACGCACC
GCCGTGATGCCCAGGGAAGACAGGGCGACCTGGAAGTCCAACTACTTCC
TTAAGATCATCCAACTATTGGATGATTATCCGAAATGTTTCATTGTGGG
AGCAGACAATGTGGGCTCCAAGCAGATGCAGCAGATCCGCATGTCCCTT
CGCGGGAAGGCTGTGGTGCTGATGGGCAAGAACACCATGATGCGCAAGG
CCATCCGAGGGCACCTGGAAAACAACCCAGCTCTGGAGAAACTGCTGCC
TCATATCCGGGGGAATGTGGGCTTTGTGTTCACCAAGGAGGACCTCACT
GAGATCAGGGACATGTTGCTGGCCAATAAGGTGCCAGCTGCTGCCCGTG
CTGGTGCCATTGCCCCATGTGAAGTCACTGTGCCAGCCCAGAACACTGG
TCTCGGGCCCGAGAAGACCTCCTTTTTCCAGGCTTTAGGTATCACCACT
AAAATCTCCAGGGGCACCATTGAAATCCTGAGTGATGTGCACTGATCAA
GACTGG
SEQ ID NO: 6
CAGCGCAGGGGCTTCTGCTGAGGGGGCAGGCGGAGCTTGAGGAAACCGC
AGATAAGTTTTTTTCTCTTTGAAAGATAGAGATTGNTACAACTACTTAA
AAAATATAGTCAATAGGTTACTAAGATATTGCTTAGCGTTAAGTTTTTA
ACGTAATTTTAATAGCTTAAGATTTTAAGAGAAAATATGAAGACTTAGA
AGAGTAGCATGAGGAAGGAAAAGATAAAAGGTTTCTAAAACATGACGGA
GGTTGAGATGAAGCTTCTTCATGGAGTAAAAAATGTATTTAAAAGAAAA
TTGAGAGAAAGGACTACAGAGCCCCGAATTAATACCAATAGAAGGGCAA
TGCTTTTAGATTAAAATGAAGGTGACTTAAACAGCTTAAAGTTTAGTTT
AAAAGTTGTAGGTGATTAAAATAATTTGAAGGCGATCTTTTAAAAAGAG
ATTAAACCGAAGGTGATTAAAAGACCTTGAAATCCATGACGCANGGAGA
ATTGCGCATTTAAAGCCTAGTTACGCATTTACTAAACGCAGACGAAAAT
GGGAAGATTAATTGGGAGTGGTAGGATGAAACAATTTTGGAGAAGATAG
AAG
SEQ ID NO: 7
CTCAAAGGAGAAAAAAAACCTTGTAAAAAAAGCAAAAATGACAACAGAA
AAACAATCTTATTCCGAGCATTCCAGTAACTTTTTTGTGTATGTACTTA
GCTGTACTATAAGTAGTTGGTTTGTATGAGATGGTTAAAAAGGCCAAAG
ATAAAAGGTTTCTTTTTTTTTCCTTTTTTGTCTATGAAGTTGCTGTTTA
TTTTTTTTGGCCTGTTTGATGTATGTGTGAAACAATGTTGTCCAACAAT
AAACAGGAATTTTATTTTGCTGAGTTGTTCTAAAAAAAAAAAAAAAAAA
AAA
SEQ ID NO: 8
AGTAGAGACGGGGTTTCACTGTGTTAGCCAGGATGGTCTCGATCTCCTG
ACCTCGTGATCCGGCCACCTCGGCCTCCCGAAAGTGCTGGGATTACAGG
CGTGAGCCACGGCGCCCAGCCCCAGCCTGTCACTTAAACTGATAAACGA
CAGATTAACAGTAGAAAAATTTTATTTTGCATACATAATGAGGCTTCAC
AAAAGAGAAGTGAAAACCCAAGTAGGAGTTTAGGGCTGGGGGCTTATAT
ACCATTTAACAAGGGGTGATAAATTGTAAGAGAATAG
SEQ ID NO: 9
TCCTTGGTTTCGATTTGTGGCAACAATCCAGTCTTTTTGTTTTTTTCAG
GGATACCATATGTAACAGGTGCCATTGTTACTGTAACTTTTCACACATG
CCTTCAGTTTGATGTCAAAGTCATCATTTAGTGTAAACAGCAAGTTATC
TGTTAGGCTGCACATCATGAACTTTACTTTTAGAAAGTCTTATCTTTTA
TGCCACAGAAATAGCATTTGGCTATTAGTCATGGATGGCAAAGAAATTA
ATTTTGAGTTGTTTGGATAAAAATGTTTCAGTTGACTGTAGTGTGTATT
GAGAGACACTGCCAGTAAACAAACTCTCTTGGTAGGTGGAAATCCCCTA
GAAGTTACAGAAAATTGGGAGGAGGTGAACTTAATTAAATAACTTGAAT
TGTTTAGACATATTCAGAGCTTCTTATGACCTTGAAGAAATCACCCAAC
TTCAAAAGACCTCGGTTTCTTCATTTGTAAAATTAGGGAGTTTGACTAG
ATGTGTAAATCTAGTTGTTAGTTAACTTCTAAGATGTAAAAACCCTCTT
GTTTAACAAAAACCTACAAGATCAAGTTGCTTATCTGAAATCTTTATGA
ATCAACACTAGTCACTAAGTCTAGCTCGACC
SEQ ID NO: 10
CTTTTCCTCCCGCTGTCCCCCACGGAGGGGACTGCTCTCCCCCGCTGCA
TCCTTTCTGTGAGGTACCTTACCCACCTCAGCACCTGAGAGGGTGAAAT
AGAATTCTAACCTCGACATTCGGGAAGTGTTTTTGAGAAGTCTCGGTCG
GTAAGGGAAGTCTTCCAAGTCCGTGCAGCACTAACGTATTGGCACCTGC
CTCCTCTTCGGCCACCCCCCAGATGAGGCAGCTGTGACTGTGTCAAGGG
AAGCCACGACTCTGACCATAGTCTTCTCTCAGCTTCCACTGCCGTCTCC
ACAGGAAACCCAGAAGTTCTGTGAACAAGTCCATGCTGCCATCAAGGCA
TTTATTGCAGTGTACTATTTGCTTCCAAAGGATCAGGCCCTGAGAACAA
TGACCTTATTTCCTACAACAGTGTCTGGGTTGCGTGCCAGCAGATGCCT
CAGATACCAAGAGATAACAAAGCTGCAGCTCTTTTGATGCTGACCAAGA
ATGTGGATTTTGTGAAGGATGCNCATGAANAAATGGACNAGCTGTG
nt: 373
SEQ ID NO: 11
AAGTGGGTCTTGCCATCCCTGAACTGNAATCATCCCTAACATATTCATA
CCTGTTTTCATTTTAAAAGTTGGGTCAGTTTTTTTATTAGTACATGTAT
TTCTATCCTACTGATTTATTTGCTATATCATCTAATTTAGTTTGAATAT
TCCATAATTTACTTAATTAGTCCTGTATGGAGACCTAGCTCTTCTCAGT
GTCTACTATTATAAACAATGCTACAGTGAATATTGGTGNATAAATCCAT
ACNCACCACGTACATATCTTAAGTTCTGGAAGAGATATTGCTAAACCAG
AAGATAACCTGCATTTAAAATTTGACTGCTAGGGNCAGGGNCACATTTA
ATTAAATTAGAACAANGAATGCATAATGNC
SEQ ID NO: 12
CCGGAATCGCGGCCGCGTCGACGAAAATATGTGCCCTGGCCAACTCCAC
AGGACTAGTTCTAGGCAATCTGAAGGAAACCAGAAAATGTGAATTTCTC
TTCCCTCAAAAAGCTATACTGAAGTAGTATTTAATATTCAAGTACTTGT
AAATTTGCAGAACAGTACTTTTTAATTTGACCCATGAATTCTATTTAAA
TTTGTCACTTAATATTTAGCCAAGAAGCAAACCATCTAAAAAGATTTCT
GGTTTATTTCTCCAACTCCTAATAAATAGGGTCACATATTTTTTAACTT
TTTTCTAATTTGAAAAGTAATACAGGCATATGGTATTTTAAAAATGAAA
CAACACAAAGGGATATGTTTTGAAAAGTGGTCTTGCCATCCCTGAACTG
TAATCATCCCTAACATATTCATACCTGTTTTCATTTTAAAAGTTGGGTC
AGTTTTTTTATTAGTACATGTATTTCTATCCTACTGATTTATTTGCTAT
ATCATCTAATTTAGTTTGAATATTCCATAATTTACTTAATTAGTCCTGT
ATGGAGACCTAGCTCTTCTCAGTGTCTACTATTATAAACAATGCTACAG
TGAATATTGGTGNATAAATCCTACACACCACGTAACATATCTTAAGTTC
CTGGAAGAGATATTGCTAAACCAGAAGATAACCTGCATTTAAAATTTGA
CTGCTAGGGTCAGGGTCACATTTAAATTAAATTAGAACAAGGAATGCAT
AATGTCTTCGATAGCAATCTATTCAAGGTGCACCGTGGTCACAAAGGAA
AGCAAAACTGTC
nt: 564
SEQ ID NO: 13
CCTGGNCAGAGGCCTCTATCCTGTANTGATAATTGCCATCAAAATTGTC
AAAAANGATTTAATTTCTATGGGNAATAGTCCTTTTCTTAGCTTCTGCC
NNTCACTTGCTTATTTTTTGTGTGGGAATGGGGTTGGATAAACCAATGA
ACTTTATTATAAACAAATCCCACCTATATCTANCAAATTTATATTTTCG
GTGAAATACAGATATTTGCCTTTCTGGAGTANTATAGAAGCTGTCAATA
TGTATCTACTGTACAGTACTAAATAGTATTCATTTATGAAATGAGTAGT
GTTTGGGTGGCTGGGGTTAAGGAAAAATGAGACTTGGAATTGTAGCTTT
TATCCAAGTTTTGAGTATAAATAGGGTTTTGTTTTGTTTTTTTTAACCT
AAAAACTGAAATGCCATATAGAAAAACAGCATTGTTTTTACAGTTTGTA
GTAAGTAACTTTTTAAAGATTTTATCAAAAAGAATTTTGTCTATNGTGA
GTAAAAGAAGTTCTAATAATGGCCTAATCACTGCATTTTTAAAAAACAA
AGTTCAACACAAATGACATTTGTTT
SEQ ID NO: 14
CCTCTCCTCCATCTAAAGGCAACATTCCTTACCCATTAGTCTCAGAAAT
TGTCTTAAGCAACAGCCCCAAATGCTGGCTGCCCCCGGCCAAGCATTGG
GGCCGCCATCCTGCCTGGCACTGGCTGATGGGCACCTCTGTTGGTTCCA
TCAGCCAGAGCTCTGCCAAAGGCCCCGCAGTCCCTCTCCCAGGAGGACC
CTAGAGGCAATTAAATGATGTCCTGTTCCATTGG
nt: 554
SEQ ID NO: 15
CCCGGAATCGCGGCCCGCGTCGACAACAAACCTGCATGTTCTGCACATG
TATCCAGGAACTTAAAAAAAAAAAAAGATAGTTTGTGTGTCTTAATTGA
ATAATAGTAGATTTATAGATTAAAGATCTATGGGTTTTTAATATGGATT
ANAAATCTGTGGGTTTTTGATATGGATTANAAATCTGTGGGTTTTTAAT
ATGGATTGGAAATCTGTGGGTTTTTAATATGGATTAAAAAACATCTGTG
GGTTTTTAATATGGATTAAACATCTGTGGGTTTTTAATATGGATTAAAC
ATCTGGGTTTTTAATATGGATTAAACATCTGTGGGTTTTTAATATGGGT
TAAAAATCAAAAGAAAATGAACTATTTGCTCCAGTGCAGGAAAATACAG
GCAATACTGGATACAATTAGATGGTCAGGAGCGATAACCCGGTTGCCAT
TGTTTGAAGAAGAGAATAAGGNGCTAGCATTCCTATCCGTAGATAATTT
GACAGCTAGGAAATAGGGGGAGTCTTCTATGTAGTTAGTGAAGGCTAAA
TGAACTATTATATGC
SEQ ID NO: 16
CTTTTCCTCCCGCTGTCCCCCACGGAGGGGACTGCTCTCCCCCGCTGCA
TCCTTTCTGTGAGGTACCTTACCCACCTCAGCACCTGAGAGGGTGAAAT
AGAATTCTAACCTCGACATTCGGGAAGTGTTTTTGAGAAGTCTCGGTCG
GTAAGGGAAGTCTTCCAAGTCCGTGCAGCACTAACGTATTGGCACCTGC
CTCCTCTTCGGCCACCCCCCAGATGAGGCAGCTGTGACTGTGTCAAGGG
AAGCCACGACTCTGACCATAGTCTTCTCTCAGCTTCCACTGCCGTCTCC
ACAGGAAACCCAGAAGTTCTGTGAACAAGTCCATGCTGCCATCAAGGCA
TTTATTGCAGTGTACTATTTGCTTCCAAAGGATCAGGCCCTGAGAACAA
TGACCTTATTTCCTACAACAGTGTCTGGGTTGCGTGCCAGCAGATGCCT
CAGATACCAAGAGATAACAAAGCTGCAGCTCTTTTGATGCTGACCAAGA
ATGTGGATTTTGTGAAGGATGCACATGAAGAAATGGAGCAGGCTGTGGA
AGAATGTGACCCTTACTCTGGCCTCTTGAATGATACTGAGGAGAACAAC
TCTGACAACCACAATCATGAGG
SEQ ID NO: 17
TGGTACAGATACAAACTGGACTCTCAGGACAAAACGACACCAGCCAAAC
CAGCAGCCCCTCAGCATCCAGCAGCATGAGCGGAGGCATTTTCCTTTTC
TTCGTGGCCAATGCCATAATCCACCTCTTCTGCTTCAGTTGAGGTGACA
CGTCTCAGCCTTAGCCCTGTGCCCCCTGAAACAGCTGCCACCATCACTC
GCAAGAGAATCCCCTCCATCTTTGGGAGGGGTTGATGCCAGACATCACC
AGGTTGTAGAAGTTGACAGGCAGTGCCATGGGGGCAACAGCCAAAATAG
GGGGGTAATGATGTACGGGCCAAGCACTGCCCAGCTGGGGGTCAATAAA
GTTACCCTTGTACTTG
SEQ ID NO: 18
CGCCACTTATCCAGTGAACCACTATCACGAAAAAAACTCTACCTCTCTA
TACTAATCTCCCTACAAATCTCCTTAATTATAACATTCACAGCCACAGA
ACTAATCATATTAAAAAAAAAAAAAAAAAAAAAAAAAAAAAAAAAAAAA
AAAAAAA
SEQ ID NO: 19
CAGAACAGTACTTTTTAATTTGACCCATGAATTCTATTTAAATTTGTCA
CTTAATATTTAGCCAAGAAGCAAACCATCTAAAAAGATTTCTGGTTTAT
TTCTCCAACTCCTAATAAATAGGGTCACATATTTTTTAACTTTTTTCTA
ATTTGAAAAGTAATACAGGCATATGGTATTTTAAAAATGAAACAACACA
AAGGGATATGTTTTGAAAAGTGGTTCTTGCCATCCCTGAACTGTAATCA
TCCCTAACATATTCATACCTGTTTTCATTTTAAAAGTTGGGTCAGTTTT
TTTATTAGTACATGTATTTCTATCCTACTGATTTATTTGCTATATCATC
TAATTTAGTTTGAATATTCCATAATTTACTTAATTAGTCCTGTATGGAG
ACCTAGCTCTTCTCAGTGTCTACTATTATAAACAATGCTACAGTGAATA
TTGGTGTATAAATCCATACACACCACGTAACATATCTTAAGTTCCTGGA
AGAGATATTGCTAAACCAGAAGATAACCTGCATTTAAAATTTTGACTGC
TAGGGTCAGGGTCACATTTAAATTAAATTAGAACAAGGAATGCATAATG
TCTTCGATAGCAATCTATTCCAGGTGCACCGTGGTCACAAAGGAAAGCA
AAACTGTCAATAACTTTCTTCTCA
SEQ ID NO: 20
TAGCATTTGGCCTTTTAAAACATTTGTTTATTTTTTTTCTGAGAATGGC
TAACACACTTTATTGAGGTTCGAAATTAATAAAGAAAATAAAAGAAATG
TATCTTCATTCATTCTGTATGTTAGTGTTTTAATTACCCTTAGAATATA
TGGATAAAAAATACTATTCTTTGTCTTGGAGAAGGTAAGAGTCTAGTTA
GATGAATAAGGGTTATCTATGTAGAACAACTAGAGAATGAGAAGAGAGC
TTATGAGATTGAGTACTACGTTATGCAGTAGAGTAGCACGTCATCTGCT
ACTGAGTATGGTGTGATAACATTGTGTAACAGGAAAGTATGATCAATAT
CTACTTAAAATTAAGGACAATATTAGCACTACATTGCTTTATTTTAAAG
TAAAAATTAGAGAACTAAACACAAGCATTGTAAGTACAATAAAAGCTGA
TCTTTCTAGTTAAGCAGAATAATACATGTTCAAGCATCTGCTAAATCAT
TAAATATAAGAATATAGGGGTTTTCTATAATCTTATTTTCTTTGGAAGA
GTACCTCATTTTCAAGANGAGAAGTTTCTAATTGCCACTTCTTTAAAAA
TAAAACAGGGTTTTAATGTTCCCAGCACAAAAATTAATATCTCTTCAAA
AAGTCTCTTGTGATTAAGTTTGAATCCCTTGTCATACTGCTTCTAATAT
TGACACTGACCTCCTTAGGTATTTTTCAGGGGTTATAATCTTTTCTTAA
GGTATCTTTTTTCAAGAATTGGATACCTTGGGCTT
SEQ ID NO: 21
CGCGTCGACTTTTAAAGTCATCTCTATAGGAAGGTGCTGGGCAGGGATC
CCAGAGAAAGAAAGGGTCCAAGACTCCATTAACTGCCCTGGATGAAGGG
CACTGCTACAGCAGCTAGTACCAGAGACTCTCCTATCTCACGGTTGAGG
CAGACCCAGGATAGAATAGAGAATAAAAGGAATGCTTATAGGAAACAAT
TTTGTATGGAATGCTAGATGGCCAAGCCTCAGCCTTTGGTCCAGTGCAA
CCCTTGCCTCGCTTGTCAACAGTGAAAAATTAGTTTGGTTAGAAGAACC
ATCTGGAAACACACCAGCTTCTGCTACCTTCATGCTCATTGTTAAAAAA
AGATTAACCAGTGTGAACATTCTGATCTGTTAATTCCAGGGACTGTTTT
CTTTCCAATGGACTGTTTGTTGGTAGAATAACCCCCAAAAGCTCAAAGC
TAAAATGCATCATCAGTCCTAGTCGGCAGTTCCTTAAGAATGGACTGGC
GGCGTGGTTGAGCTGATATGGAAAAGCTGCACCTTCCTGCAGAAGATCA
ACTGACCTGCTATCCCACCCCAAATTCAACCTGAGGTATATTTCAGTGA
AGCAGGTAGCTGTGCTTCTCAAAGCAGAGAAGCAGTTTTAAGAACCAAA
AAGGTAGAGGAAATCTA
SEQ ID NO: 22
GTTTGTTACAGGCAGAATTGGATAGATACAGCCCTACAAATGTATATGC
CCTCCCCTGAAAAAAATTGGATGAAAATCTGCACAGCAAAGTGAAACAC
ACAGATAATAGGAACAAAATGTAGTTCCCATGTGCCAAACAAAATAAAT
GAAATCTCTGCATGTTTGCAGCATATCTGCCTTTTGGGAATGTAATCAA
GGNATAATCTTTGGCTAGTGTTATGTGCCTGTATTTTTTTAAAATGGTA
CACCAGAAAAGGACTGGCAGTCTACTTCTACCATAGTTAAACTTCACCC
TCTTTAATTTCACAACATATTCTTTGGAAGCAGGAAGAAATGCTCATAA
AGAGGATCAGACCTTCTTTCCCGTGAAACCAGTATTTGGCGCCATATAT
AAGCCTGGTTAAATTGGTCATCTAAAGCTGTCAAATAAGACATTCTGTG
AAAGGTAAACATCGAAACTGGTTATAAGTAAAACCATCAAGCCAACAAC
AGGGTCTTGAGATAACCTTTGAAGCTTATTGTCTGGCCTGCACCAGAAG
ATGTCTGCATTACTCATTGCTAAAAATGTGTACACAGAACTGCACTAGG
ATTAATTGGTTCAAGAAGAAATTTAAACTTACGTTTGGGTTTCCATACA
GCACTCTATTGAATACATGCATCTGAATTTAAGTTGCAA
SEQ ID NO: 23
GACCAGTAATGGCTTTTAAGAGTCCATTTTGTCATTGTCTCCCTAGTTA
ATTACAGGTGGGGGATCTTTTGCCTCTATTCTCTTCATATTGAAATGAA
TCATACTCATGTTTTGTGGAACTCCTTAAAGTTGTAGCTGTCATGATCA
GATTTTTTTTATATTTCCTCAGCTTAACTCTGCTACTTGATTTACAGTG
ACCCATAACCTACTCATCCTTGGTTTATAGTGACACATAATCTTATCTC
TTTATAGAACCTTAAATTTTATCATTATTTTCGCTTAGAATACAGCATT
TCTTTGCTTCTGTTGCTGGTTTGACTTAAGAAATAAGGCAGTAACTCTG
ATCAATCAATTATCCATAAGGAAGGGCTTTTCATGGGTTCTATTAATTT
GTTAGTACCCTAAGTATATCTGAAAAATATGTCTATTGAGAGAAGATTT
TGGCATTCCAGATGGTATAGTCTATATATATTTAAAGTTTTGAATTTGC
TTATATATACTCAGCTTTCTTTTTCTAGCATTTTTGCATTTACCTGTTA
ATTGAAGTATACCCCCCACATATAAAAGTTCCTCTTAAAGACACTGGAC
TCTTTCTGGGGGGCTAAAATA
nt: 554
SEQ ID NO: 24
CCCGGAATCGCGGCCCGCGTCGACAACAAACCTGCATGTTCTGCACATG
TATCCAGGAACTTAAAAAAAAAAAAAGATAGTTTGTGTGTCTTAATTGA
ATAATAGTAGATTTATAGATTAAAGATCTATGGGTTTTTAATATGGATT
ANAAATCTGTGGGTTTTTGATATGGATTANAAATCTGTGGGTTTTTAAT
ATGGATTGGAAATCTGTGGGTTTTTAATATGGATTAAAAAACATCTGTG
GGTTTTTAATATGGATTAAACATCTGTGGGTTTTTAATATGGATTAAAC
ATCTGGGTTTTTAATATGGATTAAACATCTGTGGGTTTTTAATATGGGT
TAAAAATCAAAAGAAAATGAACTATTTGCTCCAGTGCAGGAAAATACAG
GCAATACTGGATACAATTAGATGGTCAGGAGCGATAACCCGGTTGCCAT
TGTTTGAAGAAGAGAATAAGGNGCTAGCATTCCTATCCGTAGATAATTT
GACAGCTAGGAAATAGGGGGAGTCTTCTATGTAGTTAGTGAAGGCTAAA
TGAACTATTATATGC
SEQ ID NO: 25
CGGCTACCGACAGAAGGACTATTTCATCGCCACCCAGGGGCCACTGGCA
CACACGGTTGAGGACTTCTGGAGGATGATCTGGGAGGGGAAGTCCCACA
CTATCGTGATGCTGACGGAGGTGCAGGAGAGAGAGCAGGATAAATGCTA
CCAGTATTGGCCAACCGAGGGCTCAGTTACTCATGGAGAAATAACGATT
GAGATAAAGAATGATACCCTTTCAGAAGCCATCAGTATACGAGACTTTC
TGGTCACTCTCAATCAGCCCCAGGCCCGCCAGGAGGAGCAGGTCCGAGT
AGTGCGCCAGTTTCACTTCCACGGCTGGCCTGAGATCGGGATTCCCGCC
GAGGGCAAAGGCATGATTGACCTCATCGCAGCCGTGCAGAAGCANCAGC
AGCAGACAGGCAACCACCCCATCACCGTGCACTGCAGTGCCGGAGCTGG
GCGAACAGGTACATTCATAGCCCTCAGCAACATTTTGGAGCGAGTAAAA
GCCGAGGGACTTTTANATGTATTTCAAGCTGTGAAGAGTTTACGACTTC
AGAGACCACATATGGTGCAACCCTGGAACAGTATGAAATGTGCTACAAA
GTGGTACAAGATTTATTGATATATTTCTGATTATGCTAATTTCAATGAA
GATCCTGCCTTAAATATTTTTTAATTTAATGGCANAT
SEQ ID NO: 26
CAAGACTCCATCTCAAAAAAAAAAAAAAATCTACAGTGCTGAGTATATA
AAATTATTAACACATTTCACAACAATATGTGTTTGTGGAGTTAAATATT
TTTTGTCTTTAAAACAGGTAATTTTAGTGCATACTTAATTTGATGATTA
AATATGGTAGAATTAAGCATTTTAAATGTTAATGTTTGTTACATTGTTC
AAGAAATAAGTAGAAATATATTCCTTTGTTTTTTATTTAAATTTTTGTT
CCTCTGTAAACTAAAAGAACACGAAGTAATTGGTCACAATTACTGGTGT
TTAACTGCCAAATATGGGTAAATAAGGGAAAATTTTGTTTAATATTTAG
TCCTTCTGAGATGGCTTGAATATTTGAATTTTGTTGTACGTCTATACTG
GGTAGTCACAAGTCTTATAAACACTTTAGAGGAAAGATGGATTTCAGTC
TGTATTTTTAAACATCATTTATTTTAAATCTGGTGCTGAAAAATAAGAA
AAAAATTAAACTGCATTCTGCTGTTCTTCTTTANAAGCATTCCTGCGTA
AATACTGCTGTAATACTGTCATGCAAAGTGTATCCTTTCTTGTCGTATC
CTTTTTGGGGCAGTGGTTTTT
SEQ ID NO: 27
GCGGGAATCGCGGCCCGCGTCGACCTCAAAGGAGAAAAAAAACCTTGTA
AAAAAAGCAAAAATGACAACAGAAAAACAATCTTATTCCGAGCATTCCA
GTAACTTTTTTGTGTATGTACTTAGCTGTACTATAAGTAGTTGGTTTGT
ATGAGATGGTTAAAAAGGCCAAAGATAAAAGGTTTCTTTTTTTTTCCTT
TTTTGTCTATGAAGTTGCTGTTTATTTTTTTTGGCCTGTTTGATGTATG
TGTGAAACAATGTTGTCCAACAATAAACAGGAATTTTATTTTGCTGAGT
TGTTCTAAAAAAAAAAAAAAAAAAAAAAAAAAAAAAAAAAAAAAAAAAA
AAAAAAATTTTAAAATTTTTAAAATAAAACCCTTGGTTAT
SEQ ID NO: 28
GCCGCGTCGACCTGCATGAGCCACAGTTTCTTGACTGGAGGCCATCAAC
CCTCTTGGTTGAGGCCTTGTTCTGAGCCCTGACATGTGCTTGGGCACTG
GTGGGCCTGGGCTTCTGAGGTGGCCTCCTGCCCTGATCAGGGACCCTCC
CCGCTTTCCTGGGCCTCTCAGTTGAACAAAGCAGCAAAACAAAGGCAGT
TTTATATGAAAGATTANAAGCCTGGAATAATCAGGCTTTTTAAATGATG
TAATTCCCACTGTAATAGCATAGGGATTTTGGAAGCAGCTGCTGGTGGC
TTGGGACATCANTGGGGCCAAGGGTTCTCTGTCCCTGGTTCAACTGTGA
TTTGGCTTTCCCGTGTCTTTCCTGGTGATGCCTTGTTTGGGGTTCTGTG
GGTTTGGGTGGGAAGAGGGCCATCTGCCTGAATGTAACCTGCTAGCTCT
CCGAAGCCCTGCGGGCCTGGCTTGTGTGAGCGTGTGGACAGTGGTGGCC
GCGCTGTGCCTGCTCGTGTTGCCTACATGTCCCTGGCTTGTTGAGGCGC
TGCTTCAACCTGCACCCCTCCTTGTCTCATAGATGCTCCTTTTGACCTT
TTCAAAATTAATATGGATGGGAAAGCTCCTATGCCTTTTGGCTTCCTGG
TAGAAGGCGGGATGCCCAAGGGTCTGCCTGGGTGTGGATTGGATGCTTG
GGGTGTGGGGGTTGGAAACTGTCTTGTGGCCCACTTGGGCCCC
SEQ ID NO: 29
CCCGCGTCGACTTTTAAAGTCATCTCTATAGGAAGGTGCTGGGCAGGGA
TCCCAGAGAAAGAAAGGGTCCAAGACTCCATTAACTGCCCTGGATGAAG
GGCACTGCTACAGCAGCTAGTACCAGAGACTCTCCTATCTCACGGTTGA
GGCAGACCCAGGATAGAATAGAGAATAAAAGGAATGCTTATAGGAAACA
ATTTTGTATGGAATGCTAGATGGCCAAGCCTCAGCCTTTGGTCCAGTGC
AACCCTTGCCTCGCTTGTCAACAGTGAAAAATTAGTTTGGTTAGAAGAA
CCATCTGGAAACACACCAGCTTCTGCTACCTTCATGCTCATTGTTAAAA
AAAGATTAACCAGTGTGAACATTCTGATCTGTTAATTCCAGGGACTGTT
TTCTTTCCAATGGACTGTTTGTTGGTAGAATAACCCCCAAAAGCTCAAA
GCTAAAATGCATCATCAGTCCTAGTCGGCAGTTCCTTAAGAATGGACTG
GCGGCGTGGGTGAGCTGATTTGGAAAACTGCCCTTCTGCAAAAAACACT
GGCCTGCTTTCCA
SEQ ID NO: 30
CAAGACTCCATCTCAAAAAAAAAAAAAAATCTACAGTGCTGAGTATATA
AAATTATTAACACATTTCACAACAATATGTGTTTGTGGAGTTAAATATT
TTTTGTCTTTAAAACAGGTAATTTTAGTGCATACTTAATTTGATGATTA
AATATGGTAGAATTAAGCATTTTAAATGTTAATGTTTGTTACATTGTTC
AAGAAATAAGTAGAAATATATTCCTTTGTTTTTTATTTAAATTTTTGTT
CCTCTGTAAACTAAAAGAACACGAAGTAATTGGTCACAATTACTGGTGT
TTAACTGCCAAATATGGGTAAATAAGGGAAAATTTTGTTTAATATTTAG
TCCTTCTGAGATGGCTTGAATATTTGAATTTTGTTGTACGTCTATACTG
GGTAGTCACAAGTCTTATAAACACTTTAGAGGAAAGATGGATTTCAGTC
TGTATTTTTAAACATCATTTATTTTAAATCTGGTGCTGAAAAATAAGAA
AAAAATTAAACTGCATTCTGCTGTTCTTCTTTAGAAGCATTCCTGCGTA
AATACTGCTGTAATACTGTCATGCAAAGTGTATCCTTTCTTGTCGTATC
CTTTTTGGGGCAGTGGTT
SEQ ID NO: 31
CTGGACTGCATGACCAGATCTGATGGGTGAGACTCAGGTGGCATGGAAG
AGCCGAAAGAGGATACCATATGTGGGTGCCGGGGGGGATAGGTGAGAAG
TACTAGAAGGCGGAATGGAAGGACACTTCTGCTCAGCTCTGTGACACGG
GCAGGGACCCTGCAGGGCTCAGGTCCTTTAACACAGCAGCTTCATTCTA
ACACCAGCAGCGTTGGAACACACGTACAAGTATGCAGACTAAGCTCTTG
CTTGGCTGATACGGCTTTTTGGGTTTTTAGAGAACATGCATATATGTTC
TCATTCATGGTACATGAACTCAGAAGCCTTACTGCCTATTTTTGTTAAT
ACTTCTGGGCAAACATTACCACTTACAACTCACACCAGTTAGAAATCAT
TTGTAAAATGTTATTTAATAAAGCCAAAGAACTAAATCATATTTATTTT
CCAAGGNTTTCTAAGATCTCTGAAACTAATGAGGTTTTTTAAATCCCCA
TTAAGTACTCATCACTGCTAGTAAAAGCAGTTGTCTTTACCTTTAATTC
CAGTGAGTCCCCTTAAATTTATTTTTTATTATCTTTGGCTACATTGCCT
TAGACAAAATGTGGTCACCCTAATTTAANGGATAAAATTCACATCCTCA
CAGATTTCTTATTAAGAGGGTCTAANCCTTGAATAATCANCAGTGGAAA
TGGAAGTCTTCTTTACTGGNTTTNATCCTTTCCCTTTTTTATCCCATG
SEQ ID NO: 32
TTTTTTTTTAAATAAAGCTGTCGGCACTCAAGGGTAATTTCATATCAGT
GTGNTCTACAAGCTGGGGGAAAATGAGTTCTAATTGTCANAGCTACCAA
ATCCTTCACCTTTAGCATAAAGGTTTAAAGATATCACAAAGATGCCAAG
TGATTAATAATGTTTTAAACCACCCCTTTTTCTGTCTGAAAAAACAACT
AAAACAATATTACAACAGTATAGTTACAGAAGGGTTCTATTTTCATATG
TTTTATGCACACTGTGCCTCAAAGGTACTATTTAAATATATATACTTTT
GAGGGGGTGGCTAATGCAGAAACACCCAAGACCTAAGGAAGATACAACC
CCATTTCTAGGTGTGAGGTCTAAATGCTTCACACACCCACTTGTGACCT
TTTTTCATGAAGAATCATAACACTGTGCAGTGAGAAACAGTGGCAAAGC
AATACTGAAAGCATTTTAAATTATTTACTAGGTTAAAAGGGTGAACTGA
TACTTTAAATACATCAAATTTCATCAT
SEQ ID NO: 33
GCAAGTGAGAGCCGGACGGGCACTGGGCGACTCTGTGCCTCGCTGAGGA
AAAATAACTAAACATGGGCAAAGGAGATCCTAAGAAGCCGAGAGGCAAA
ATGTCATCATATGCATTTTTTGTGCAAACTTGTCGGGAGGAGCATAAGA
AGAAGCACCCAGATGCTTCAGTCAACTTCTCAGAGTTTTCTAAGAAGTG
CTCAGAGAGGTGGAAGACCATGTCTGCTAAAGAGAAAGGAAAATTTGAA
GATATGGCAAAAGCGGACAAGGCCCGTTATGAAAGAGAAATGAAAACCT
ATATCCCTCCCAAAGGGGAGACAAAAAAGAAGTTCAAGGATCCCAATGC
ACCCAAGAGGCCTCCTTCGGCCTTCTTCCTCTTCTGCTCTGAGTATCGC
CCAAAAATCAAAGGAGAACATCCTGGCCTGTCCATTGGTGATGTTGCGA
AGAAACTGGGAGAGATGTGGAATAACACTGCTGCAGATGACAAGCAGCC
TTATGAAAAGAAGGCTGCGAAGCTGAAGGAAAAATACGAAAAGGTA
nt: 622
SEQ ID NO: 34
CTGTNATNGAATCTGCTTGTNACTNAAATGCTAAACTCAATTCTGTAAT
TCAATAGGTGCACCTNTCTGAGAAACATANNAGACAATGAGGAAAAGGA
TTCANCATTCCGTGGAATTTGTACCATGATCAGTGTGAATCCCANTGGC
GTAATCCAAGTAAGATGTTCACAAAGATTTGTTTTTAATGTCTAATTAA
TAAAATTTTAAAGGAAGAAACATTCTAATACTTTAATTATAAAAAGTTA
ACTATTTTCAAAGGTATCAAAATACAGTTAAACCTTTAAAATGTATATT
TCTTAATATCTTGAAATTGTAATGCCTTTTTTTTTTCCTAAATTTTTTT
TGTCATGAAATGAGATAGTAACAGCAGATTGGGACAACAAGGTTATATT
CTTGTCTTGAATCAGGCCATGGCTTCTTTCATCCAAATTTCAGACCTCA
TTTATTTACTTTGTCCCTGCCTCCCATCCCTGGATATCANGTTTGTGGA
TATCTACAGTTAATAGAGTGACCAAATAGTAGGAATACTGTCTCTCTAT
TCTGAATAAAATACTTTGAATCAGATTTAGAAATAATGAATAAAATACA
AATCACCATTGAAATTGCTCTAATTTTGAGAGCT
nt: 628
SEQ ID NO: 35
ATCACNTGAGGCAAGAGTTTGAGCCAGCCTAGCTAACATGGTGAAACCC
CATCTCTACAAAAATATAAAAATTAGCCTGGGTGGTGATGGGCACCTGT
AACCCCAGCTACTCGGGAGGCTGAGGTAGGAGAATCACTTGAACCCGGG
AGATGGAGGTTGCAGTGAGCCAAGATCGTGCCACTGCACTCCAGCCTGT
GTGACAGAACAAGACTCTGTCTCAAAAAAAAATAATAATAATAATAATA
ATAAAAAGGAATAACATAGCTAGGAATAAATTTAATCAAAGAGGTGAAA
GACTTATACACTTAAAACTACAAAAAAAAAATCACTGAAGGAATTATAG
ACCCAAATAAAAATAAATAAAAAGACATTCTGTGTTTTAGGGAAAGAAG
ACTTAATATTGTTAAGATGTCAATACTACCCAAAGTGATCTACAGATTC
AACATAATCCCTATCAAAATTCCAACAGCCTACTTTGTAGAAATGGAAA
AGCCAATTTTCAAATTCAGATGGAATTGCGAGGGGTTCTGAATAACAAA
AACAATCTTGGGGAAAAAAAACAAAAAACAAAGTCAAAGAACTCACACT
TCTCTATTTATAAATTTACTACAAAGTTATAGTAATCAAA
nt: 527
SEQ ID NO: 36
TGAACATCCAGCCATGTCATTTCTTCCATTCCTGCCCTGGAGTAAAGTA
GATTTACTGAGCTGATGACTTGTGTGCATTTGTACATTGCAACCTTAGC
TTACCTCTTGAAGCATGTAGAGCATTCATCACCCACCATTCATTCACTG
CCTACTCCCACCACAGCTGTTTCGTGGTCTGTCTGCTCCCTGTGCCACC
CCCACCCCATCAGGTGGGCCTTTTGCAAGTGATGAAGTCACCTGTGGGG
GAAGAGCTTTCCTTTCCTCTCCTCAACTCAGAAGGCCTCTTCCTCTTGC
TCAAGAGGGTGCTGCTGCTTTCTGCCTCCTTCCCCGGCCGGCCTCCATC
CCAGTTCACCTTTTCAGAAATGGCCCCTCAGTCAACTCTTCCCTTTTCT
CCTGGCTTTTTATTTCTCCCAGTCTCTTAAGAGTATCCTTAGCTTTAAA
AACAATAACACAGAGGATGGGTGCAGTGGCTCATGCCTGTAATCCCAGC
ACTTTGGAGCCTGGGGCGGGCGGATCACTTGAGGNCA
SEQ ID NO: 37
GTCCCGGAATCGCGGCCGCGTCGACCTTTTCTATGCCTGCTATATAAAC
AGTACCTTGCAAGATGTCCTGTCTGATATCCACAAAGGGGTATTGTCAA
CCCCAAGTTCAGACAGCTTTGTATTCTTCTGTCCCTGGATACATGAATT
ACTGCCATCTTTACACAGCGCCCTAAAATACCAACGCGAAGTTACCTGC
TCAGCTTGAAGCTGCGCTGTACCCTGGAACCAGCACTTCTGCTGAATGA
CTCAGGATGAAGCCTCGACTTCTCCTTCCCATCCCATGCCCAGACCCCA
GTGGCTCCTTTCCCAATCTGATCCAGTGACTTTAAGTCCAGCTGTTGCA
ACCTGGGCATGAGGAGGAGTGCAAGATGGCTTTGTCCTACCTGGAAAGA
GGCTTTCTGGA
SEQ ID NO: 38
CACCATTTACACACAGTGGGTCCTTGAATAGCATCGTTTTATTCAATGT
CATTTTGTTATAACATTGAGAAAAAAATTGATTCCCGGCTGGGGCCACT
GTCTGTGCACCGT
nt: 329
SEQ ID NO: 39
GAAAGATCTAAAATCGACACCCTAACATCACAATTAAAAGAACTAGAGA
AGCAAGAGCAAATTCAAAAGCTAGCAGAAGGCAAGAAATAACTAAGATC
AGAGCAGAGCTGAAAGAGATAGAGACACAAAAAACCATTCAAAAAAAAA
CAATGAATCCAGGAGTTTTTTTTTTAAAAAGATCAACAGAATTGACAGA
CTGCTAGCAAGACTAATAAAGAAGAGAGAAGCATCAAATAGACTCAATA
AAAAATGATAAAGGGGATATCACCACCAATCCCACAGAAATACAAACTA
CCATCAGAGAACACTATAAACACCTCTATGCAAAT
SEQ ID NO: 40
GAAAGATCTAAAATCGACACCCTAACATCACAATTAAAAGAACTAGAGA
AGCAAGAGCAAATTCAAAAGCTAGCAGAAGGCAAGAAATAACTAAGATC
AGAGCAGAGCTGAAAGAGATAGAGACACAAAAAACCATTCAAAAAAAAA
CAATGAATCCAGGAGTTTTTTTTTTAAAAAGATCAACAGAATTGACAGA
CTGCTAGCAAGACTAATAAAGAAGAGAGAAGCATCAAATAGACTCAATA
AAAAATGATAAAGGGGATATCACCACCAATCCCACAGAAATACAAACTA
CCATCAGAGAACACTATAAACACCTCTATGCAAATAAACTAGAAAAT
SEQ ID NO: 41
GAAAGATCTAAAATCGACACCCTAACATCACAATTAAAAGAACTAGAGA
AGCAAGAGCAAATTCAAAAGCTAGCAGAAGGCAAGAAATAACTAAGATC
AGAGCAGAGCTGAAAGAGATAGAGACACAAAAAACCATTCAAAAAAAAA
CAATGAATCCAGGAGTTTTTTTTTTAAAAAGATCAACA
SEQ ID NO: 42
GCCCGGAATCGCGGCCGCGTCGACGTAAGCTCGGCTGAATCCACGGTTC
AAGAACAGGAAAGAAGGCCAAGGCATAGGGAGTGGGGCAGTTGGGTGAA
TATTAGTACCTTTCCCTCAGNTNCATTAATTACCCCTGCCTACTCTGCA
CAAAAGGATNTAACAACAGTTTCCTTTTTAATGGCCAGGTACAGCTGCT
TATATGGANGGGCATTTNTNAATGATATCCTTNATCACTGTCTTAATCA
TCACATNCTTAAAACAATCACTTTATTGTGTTAAGGAAGATAAAAATGG
CTGGGTTCAATTTCCGTTCTGGAAGAAATCGANTNAAAAGGTAACCATT
TAATAATGCANAGGGCANTTTCACTGCAGACCCTAATACTGGAAATTTT
TAAAAACAAATGAAAAACTTCTACTTTTTCTTCTAAGCTTACTTAACCA
CCCAAATTTTCCAGCCACATATCTTCCTAGTCTACAACTGCCTTTAACT
TTAAGAGATGCTCAAAAAAATGTAAATTCTCAAATACATTCTTATTACA
ATTACTGCTAACCT
SEQ ID NO: 43
CCAGTGTGCTGGGATTACAGGCATGAGCCCTGCACCCAGCCTCTTAAAC
TGATCATATGATATTGGTTCTCAACCAAGGGTGACTTTGCCCCCAGAGG
ATACTTGGCAATGTCTGGAGATACTCAGTTGTCATGACTTGGACAGGTG
CTACTGTCACCCAGTGGGTAGAGGTCAGGGATGGTGCTAAACATAGGAC
AGCTGTCAAGAGAAAAGAATGTACCCAGCCCCAAATGTCAGTAGGGCTG
AGGTTGAGAAACCCAGCTGTAGCTGACGTGTGAAGGACAGACTGGCCTG
GAAGTGTGTTTTCTGCCCCTTTCCACCCCTGCATATTAGTTAAGGCCAA
AGGAAAAAAGGAATGCAGGAAATGCCCGTTAAAAATCTTCAAAACAATA
TAAAATGATCAATTCCACTAAAACCCTTTACACATTTAAGTATAAAGGT
ATTGGTAGGAAAATTTGTTATTCACTGCTTTTCTCAGTGTCATGAAATA
ATTATTTCTGCTGTCAGTTT
SEQ ID NO: 44
AAAAAAAAAATCACTGAAGGAATTATAGACCCAAATAAAAATAAATAAA
AAGACATTCTGTGTTTTAGGGAAAGAAGACTTAATATTGTTAAGATGTC
AATACTACCCAAAGTGATCTACAGATTCAACATAATCCCTATCAAAATT
CCAACAGCCTACTTTGTAGAAATGGAAAAGCCAATTTTCAAATTCAGAT
GGAATTGCGAGGGGTTCTGAATAACAAAAACAATCTTGGGGAAAAAAAA
CAAAAAACAAAGTCAAAGAACTCACACTTCTCTATTTATAATTTACTAC
AAAGTTATAGTAATCAAAGTCGACGCGGCCGCGATTCCGGG
SEQ ID NO: 45
CGACTGCGGCTCTTCCTCGGGCAGCGGAAGCGGCGCGGCGGTCGGAGAA
GTGGCCTAAAACTTCGGCGTTGGGTGAAAGAAAATGGCCCGAACCAAGC
AGACTGCTCGTAAGTCCACCGGTGGGAAAGCCCCCCGCAAACAGCTGGC
CACGAAAGCCGCCAGGAAAAGCGCTCCCTCTACCGGCGGGGTGAAGAAG
CCTCATCGCTACAGGCCCGGGACCGTGGCGCTTCGAGAGATTCGTCGTT
ATCAGAAGTCGACCGAGCTGCTCATCCGGAAGCTGCCCTTCCAGAGGTT
GGTGAGGGANATCGCCCAGG
SEQ ID NO: 46
GCAATTTAATTTTTAATAACAAAGATACTGTATTTTAACATGGTGAAAT
ATACTTGGCTAAGTCCAGATTAAAAAAAAAAAGTATCTAGCCCAACAGT
ACAATTATACAGCTTTGTACAGAACATTCCATAGATCAACAGAAAATAC
ATTTGAGCGCAAAAATAAAAAATATTTAAGGAGAATCTCTAAGCAGCAT
TTTATTTCTGCAAAAGACATATCTTGTCTGATTAAATATCTACAAGTGC
TTTTCCTTTCAAAAATACATATATTCTTAATAGACTAAGTCATTAACAA
TGACCTGGTAATTCTTTCACTTCAATTTGAATGATTTATAAGCTAAATC
TTCAACCACAAAAAGGTTTTTATTTGTATTAAGATGTTACCACTTTTGA
CAAAAAGCTTAAAATATTTTATATTTCAAAGGAAAATTAGCAACATAAC
TTTACAATATATTCTATGATATTTTGATTGTGAGGGCTACTCTATTTAA
AACTGATGATCTCTGTTGTGTTGCTCAGATGCAGGAAAGCAGCAAAA
nt: 534
SEQ ID NO: 47
GACTTANATCTAAATGGACCACATTCTCTACTTAAAAAAATGCTATTAA
CCATGTGATCTTCTCAGTCATGAGGTAATCTGGTGACTACCCTTCCTCA
AAGCCAGTTGGGATATTCTTTGAATAGAGTAAAACAGTGTTTCTAGGCT
GGGAGACACCAGACATAGTTGAGGACAGAGGTGCTAGAAAATAGGAAGT
TTAAAAGCATGTGCGGTGATGCTCAGAGGAGGTAAACCCCACCCTCATG
CTCATAGCTTCCAATCATTTTCTCTAGTTCTTAACTCTTAAATGTGAGA
AATGCTTGAAGATTCTAGTCATCTGAAGAAAGTCTCTTTATTAAAGATT
TTCATAAAAGAGACCAAAGCAGACAAACAGAAAAAGACATCTTGGGGAA
AAAAACAAGGATAATGGGAAGAGAAGGAAAGTTTTAAAAATTATCAATA
TCCTCAGGGGGACAAAATATTATATCCTATAAAGACAGATTTTTATTTT
TTAAAAAAATAGAAAGCAAAACAAGCTCCTAAAAATAAAGTTTG
nt: 444
SEQ ID NO: 48
GTTAAGGAAGTCAGCACTTACATTAAGAAAATTGGCTACAACCCCGACA
CAGTAGCATTTGTGCCAATTTCTGGTTGGAATGGTGACAACATGCTGGA
GCCAAGTGCTAACATGCCTTGGTTCAAGGGATGGAAAGTCACCCGTAAG
GATGGCAATGCCAGTGGAACCACGCTGCTTGAGGCTCTGGACTGCATCC
TACCACCAACTCGTCCAACTGACAAGCCCTTGCGCCTGCCTCTCCAGGA
TGTCTACAAAATTGGTGGTATTGGTACTGTTCCTGTTGGCCGAGTGGAG
ACTGGTGTTCTCAAACCCGGTATGGTGGTCACCTTTGCTCCAGTCAACG
TTACAACGGAAGTAAAATCTGTCGAAATGCACCATGAAGCTTTGAGTGA
AGCTTTTCCTGGGGACAATGTGGGCTTCAATGTCAAGAATGTGTCTGTC
AAG
nt: 566
SEQ ID NO: 49
CTTTGAAGAACTTTGCCAAATACTTTCTTACCAATCTCATGAGGAGAGG
GAACATGCTGAGAAACTGATGAAGCTGCAGAACCAACGAGGTGGCCGAA
TCTTCCTTCAGGATATCAAGAAACCAGACTGTGATGACTGGGAGAGCGG
GCTGAATGCAATGGAGTGTGCATTACATTTGGAAAAAAATGTGAATCAG
TCACTACTGGAACTGCACAAACTGGCCACTGACAAAAATGACCCCCATT
TGTGTGACTTCATTGAGACACATTACCTGAATGAGCAGGTGAAAGCCAT
CAAAGAATTGGGTGACCACGTGACCAACTTGCGCAAGATGGGAGCGCCC
GAATCTGGCTTGGCGGAATATCTCTTTGACAAGCACACCCTGGGAGACA
GTGATAATGAAAGCTAAGCCTCGGGCTAATTTCCCCATAGCCGTGGGGT
GACTTCCCTGGTCACCAAGGCAGTGCATGCATGTTGGGGTTTCCTTTAC
CTTTTCTATAAGTTGTACCAAAACATCCACTTAAGTTCTTTGATTTGTC
CATTCCTTCAAATAAAGAAATTTGGTA
SEQ ID NO: 50
TTTTGGGGTTTATATATAAGCCTGGTTCTTGCTGAAACTGCTTATGTTG
ATAACCAGTTAGTGAGTTCCTCTCTATTGACTTGCTGGGAAGTTTATAG
AGACATTTTTTATGCATTCAGAGATTTCAGTACAAATCTTGAAAAAGGG
ACATTTAGGCCGGGCGCGGTGGCTCACATCTGTAACCCTAGCACTCTGG
GAGGCTGAGGTGGGTGGATCATGAAGTCAAGAGATAGAGACCATCCTGG
CAAAAATTAGCTGGGCGTGGTGGGGTGCGCCCGTAGTCCCAGCTACTCG
GGAGGCTGAGGCAGGAGAATTGCTTGAGCCCGGGAGGCGGAGGTTTCAT
TGAGCCGAGATAGTGCCACTGCACTCCAGCCTGGACAACAGAGCGAGAC
TGTGTCTT
SEQ ID NO: 51
CTAAGGGTTTAAAGATGGAAAGAGGCATTGATGAACAGCTGGGGAAGGA
GTAGTTTGAGGTAGATGTGCAGATGGAATGAAGAGAAGGTCTCAAGAAG
AGGGTGGAGCCAAAGAGGGCTGCAGATTTAGAAGGCTAAAGTCTTTAGA
TGGCTTTGGATAGCCTGTTGTATCTTGGACCATGCAGGTTACAGTGGAG
CATGGAGTGGGGACAGAAGTGGAGGAAGGAACCAGGGAACATGGAGTGA
GAAGCTAAAGGAAAGTGATGCAGTAGATACATGGCTCTAAAGTACTCAG
GACTTTCAGAGGCTTAAACATAGGGTGACCAACTATCCCACTATGCCTG
ATACTAAGGGCATTCCCTGGATGTGGACCTTTCATTCCCCAAATTAGGA
AAGTCTTGGGCATACCAAGACAAGTTGGCCACCCTACTCAAAAGTATGT
AAGCTAACATATCTGTTCTCTAAGAGGTTAAAGCTGGATGGGGATACCA
GATGTATGTACGTGATGCAGTTAAACAGCAATACAAGGGGGCAAGTCTA
CCTGATCGGCCAATTCAATGGGA
SEQ ID NO: 52
GAAGCCAAACCAAAGGAGCTTCTACTTCATGATGCCATTTATGTAAAGT
TCAGGCAGAGAAAATCAGTGGTTTAAGAAGTTAGAATAATGATTATCTT
TGGAGGGATTGCAACTGGAAGAAGTCATGATTGGGATTTCTGGGTCCTA
ATAGTGCTCTGTGTCTTGATCTGAGTGCCGACTACATGAGTGGTTAGGT
TTGCAAAATTCATTGAGTTATGCACTTAATGGTGTTGTCTTATTAGAGC
TGATGGAGGAGAGAGGGCTTCAATTTGCACAACTGAGTAATCAGCTAGG
CCCAGTCACTAGGTGAACAACTTACTGCTCCAATCAGCCTTAGAGCAGG
AATCAAACTCATGTCTCAGAAAAGTTATTAATTCAGCTTGTCTTGGGAC
TTCCTTCAGAGTCACTCTTGAATAGCTGAAATAGTAAATGTTAAATCTG
TGGATGCAAGTGTGTAAATTATTTTAGTCATCAGCTCTAATAAGATGGC
CTTTGGGGAAATGAGTATAAGGTCACGAAAATGAAATGGCAAGAAGGAG
GTCTACTATTTCTTCTGTAATACTGATTTTTACCCCATCAGGGTCAGTC
CCCAGAGGTTGTAAATGTGAAGCTTG-TCTTTTTCTTTAATAA
SEQ ID NO: 53
CTTTGGACACTAGGAAAAAACCTTGTAGAGAGAGTAAAAAATTTAACAC
CCATAGTAGGCCTAAAAGCAGCCACCAATTAAGAAAGCGTTCAAGCTCA
ACACCCACTACCTAAAAAATCCCAAACATATAACTGAACTCCTCACACC
CAATTGGACCAATCTATCACCCTATAGAAGAACTAATGTTAGTATAAGT
AACATGAAAACATTCTCCTCCGCATAAGCCTGCGTCAGATTAAAACACT
GAACTGACAATTAACAGCCCAATATCTACAATCAACCAACAAGTCATTA
TTACCCTCACTGTCAACCCAACACAGGCATGCTCATAAGGAAAGGTTAA
AAAAAGTAAAAGGAACTCGGCAAATCTTACCCCGCCTGTTTACCAAAAA
CATCACCTCTAGCATCACCAGTATTAGAGGCACCGCCTGCCCAGTGACA
CATGTTTAACGGCCGCGGTACCCTAACCGTGCAAAGGTAGCATAATCAC
TTGTTCCTTAATTAGGGACCTGTATGAATGGCTCCACGAGGGTTCAGCT
GTCTCTTACTTTTAACCAGTGAAATTGACCTGCCCGTGAAGAGGCGGGC
ATAACACAGCAAGACGAGAAGACCCTATGGAGCTTTAATTTATTAATGC
AAACAGTCCTAACAAACCCCAGGTCCTAAACTCCAAACCTGCATTAAA
SEQ ID NO: 54
CGACCCGGAATTCGCGGCCGCGTCGACTGAGTTCTTGACAAGAGTGTTT
TTCCCTTCCCGTCACAGAGTGGGCCCAACGACCTACGGCACTTTGACCC
CGAGTTTACCGAAGAGCCTGTCCCCAACTCCATTGGCAAGTCCCCTGAC
AGCGTCCTCGTCACAGCCAGCGTCAAGGAAGCTGCCGAGGCTTTCCTAG
GCTTTTCCTATGCGCCTCCCACGGACTCTTTCCTCTGAACCCTGTTAGG
GCTTGGTTTTAAAGGATTTTATGTGTGTTTCCGAATGTTTTAGTTAGCC
TTTTGGTGGAGCCGCCAGCTGACAGGACATCTTACAAGAGAATTTGCAC
ATCTCTGGAAGCTTAGCAATCTTATTGCACACTGTTCGCTGGAAGCTTT
TTGAAGAGCACATTCTCCTCAGTGAGCTCATGAGGTTTTCATTTTTATT
CTTCCTTCCAACGTGGTGCTATCTCTGAAACGAGCGTTAGAGTGCCGCC
TTAGACGGAGGCAGGAGTTTCGTTAGAAAGCGGACGCTGTTCT
nt: 523
SEQ ID NO: 55
GAATCCCTAGAAAAAGAGAATTCCCAACTTGATGAGGAAAACTTAGAAC
TGCGAAGGAATGTAGAATCTTTGAAGTGTGCAAGCATGAAAATGGCTCA
GCTACAGCTAGAAAACAAAGAACTGGAAAGTGAAAAAGAGCAACTTAAG
AAGGGTTTGGAGCTCCTGAAAGCATCTTTCAAGAAAACAGAACGCTTAG
AAGTTAGCTACCAGGGTTTAGATATAGAAAATCAAAGACTGCAAAAAAC
TTTAGAGAACAGCAATAAAAAAATCCAGCAATTAGAGAGTGAACTACAA
GACTTAGAGATGGAAAATCAAACATTGCAGAAAAACCTAGAAGAACTAA
AAATATCTAGCAAAAGACTAGAACAGCTGGAAAAAGAAAATAAATCATT
AGAGCAAGAGACTTCTCAACTGGAAAAGGATAAGAAACAATTGGAGAAG
GAAAATAAGAGACTCCGACANCAAGCAGAAATTAAAGATCCACATTTGA
AGAAAATAATGTGAAGATTGGAAATTTGGAAAA
nt: 566
SEQ ID NO: 56
CTTTGAAGAACTTTGCCAAATACTTTCTTACCAATCTCATGAGGAGAGG
GAACATGCTGAGAAACTGATGAAGCTGCAGAACCAACGAGGTGGCCGAA
TCTTCCTTCAGGATATCAAGAAACCAGACTGTGATGACTGGGAGAGCGG
GCTGAATGCAATGGAGTGTGCATTACATTTGGAAAAAAATGTGAATCAG
TCACTACTGGAACTGCACAAACTGGCCACTGACAAAAATGACCCCCATT
TGTGTGACTTCATTGAGACACATTACCTGAATGAGCAGGTGAAAGCCAT
CAAAGAATTGGGTGACCACGTGACCAACTTGCGCAAGATGGGAGCGCCC
GAATCTGGCTTGGCGGAATATCTCTTTGACAAGCACACCCTGGGAGACA
GTGATAATGAAAGCTAAGCCTCGGGCTAATTTCCCCATAGCCGTGGGGT
GACTTCCCTGGTCACCAAGGCAGTGCATGCATGTTGGGGTTTCCTTTAC
CTTTTCTATAAGTTGTACCAAAACATCCACTTAAGTTCTTTGATTTGTC
CATTCCTTCAAATAAAGAAATTTGGTA
SEQ ID NO: 57
GACCCGGAATCGCGGCCGCGTCGACCATTTTAGCCAAGGTGCCTCTATA
GGGGTCAAGACATCATGTGCCCAGACCTAAGGTCAGGAATGTCATATTT
TTCTGTTAAAATCATTTTATTTCTGTGTATCTTACCTTTAAATCATTGT
GGTTTACTCTGAGATTCTGTAGTCCTAATATTGTATCATTGTGCTGTCT
GCAAAACAACTTGAATCTATTTTGTTTGCATCTTTTGTTACATGTAACG
CAGCTGTACTTTATGTTCTTTGCAACTGTTTCCATTATGAGAACGCTGT
GCTATTTACAAGGTTACATTTTTCTTGGCCAGGCGAGGTGGTCATGCCT
GTGATCCCAGCACTTTGGGAGGCCAAGGTGGGCGGATCACTTGAGGTAA
AGAGTTGAGACCAGCCTGGCTAGCATGGCGAAGCCCAGTCTCTACTAAA
AATACAAAAATTGGCCGGGTGAAATTAGCCGGGCGTGGTGGTGTGTGCT
TGTAATCCCAGCTACTCGGGAGGCTGAGGCAGGAGAATCGCTTGAATCC
GGGAGGCAGAGGTTGCAGTGAGCCAAGATCANGCCACTGCACTCCACCT
CGGGGTCAAGAGCGAAACTCTGTCTCAA
SEQ ID NO: 58
CCGTTTTAGTCAGGATGGTCTCGATCTCCTGACCTCGTGATCCGCCTGC
CTCGGCCTCCCAAAGTGCTGGGATTACAGGCGTGAGCCACCGCGCCCGG
CGTAAATCAGGTTTTTTAAATGTTTGCCAAACCTTATCACTGACTTTTA
TAACAAAATTATTTACTATAATCATTAGGGAATATTTAAGTTCTGCTAA
TACTTAAAATTGCAGAGTGCTAAAACCAGCAGTGAGTTTAGAATCAAGC
TAAGCTTTATTGTTGCTACTATTTGAGGCATATTAGTTGACTGGTGTTC
ATATGCAAGGCAGTCTACTGGGTGCAACAAGGGTTAGAAGGATATTTTT
AAAAAACTGACCCTATTCTCAGGATGAAAATAATACACTAGTAATAGTC
TGCTCTGTTGGTTAACTCCTCGTAAGGAGGTCAATTAAAATGCTGTAGT
GTTGCAAGGGAAGGAGAGGAAGAATCATATTCCTTCACTAGCAGGATCA
AGAAAGCTTTTATAGAAATATACAAAATCTTCACTTCTTGAAGGATTGG
TAAAATTTAATAGCCAACATTGGGCACTTATTCATTCTCTGAGTAAATA
TTTATTGCAT
SEQ ID NO: 59
CTTAAATCTAAATGGACCACATTCTCTACTTAAAAAAATGCTATTAACC
ATGTGATCTTCTCAGTCATGAGGTAATCTGGTGACTACCCTTCCTCAAA
GCCAGTTGGGATATTCTTTGAATAGAGTAAAACAGTGTTTCTAGGCTGG
GAGACACCAGACATAGTTGAGGACAGAGGTGCTAGAAAATAGGAAGTTT
AAAAGCATGTGCGGTGATGCTCAGAGGAGGTAAACCCCACCCTCATGCT
CATAGCTTCCAATCATTTTCTCTAGTTCTTAACTCTTAAATGTGAGAAA
TGCTTGAAGATTACTAGTCATCTGAAGAAAGTCTCTTTATTAAAGATTT
TCATAAAAGAGACCAAAGCAGACAAACAGAAAAAGACATCTTGGGGAAA
AAAACAAGGATAATGGGAAGAGAAGGAAAGTTTTAAAAATTATCAATAT
CCTCAGGGGGACAAAATATTATATCCTATAAAGACAGATTTTTATTTTT
TAAAAAAATAGAAAGCAAAACAAGCTCCTAAAAA
nt: 534
SEQ ID NO: 60
GACCCGGAATCGCGGCCGCGTCGACGGAAGCTCCTGCCCCTCCTAAAGC
TGAAGCCAAAGCGAAGGCTTTAAAGGCCAAGAAGGCAGTGTTGAAAGGT
GTCCACAGCCACAAAAAGAAGGAGATCCGCACGTCACCCACCTTCCGGC
GGCCGAAGACACTGCGACTCCGGAGACAGCCCAAATATCCTCGGAAGAG
CGCTCCCAGGAGAAACAAGCTTGACCACTATGCTATCATCAAGTTTCCG
CTGACCACTGAGTCTGCCATGAAGAAGATAGAAGACAACAACACACTTG
TGTTCATTGTGGATGTTAAAGCCAACAAGCACCAGATTAAACAGGCTGT
GAAGAAGCTGTATGACATTGATGTGGCCAAGGTCAACACCCTGATTCGG
CCTGATGGAGAGAAGAAGGCATATGTTCGACTGGCTCCTGATTACGATG
CTTTGGATGTTGCCAACAAAATTGGGATCATTTAAACTGAGTCCAGCTG
CCTAATTCTGAATATATATATATATATATATCTTTTCACCATAA
nt: 512
SEQ ID NO: 61
GGGGAGCCCCCTCTTCCCTCAGTTGTTCCTACTCAGACTGTTGCACTCT
AAACCTAGGGAGGTTGAAGAATGAGACCCTTAGGTTTTAACACGAATCC
TGACACCACCATCTATAGGGTCCCAACTTGGTTATTGTAGGCAACCTTC
CCTCTCTCCTTGGTGAAGAACATCCCAAGCCAGAAAGAAGTTAACTACA
GTGTTTTCCTTTGCACCGATCCCCACCCCAATTCAATCCCGGAAGGGAC
TTACTTAGGAAACCCTTCTTTACTAGATATCCTGGCCCCCTGGGCTTGT
GAACACCTCCTAGCCACATCACTACAGTACAGTGAGTGACCCCAGCCTC
CTGCCTACCCCAAGATGCCCCTCCCCACCCTGACCGTGCTAACTGTGTG
TACATATATATTCTACATATATGTATATTAAAACTGCACTGCCATGTCT
GCCCTTTTTTGTGGTGTCTAGCATTAACTTATTGTCTAGGCCAAAGCGG
GGGTGGGAGGGGAATGCCACAG
SEQ ID NO: 62
TTTTGGCATTACTTAATCCAATTATAAAAACTGAATTTTTAAAAAACAG
CACTTGTTTTTTCTTCCAAGATTAATTTGAATTTTTTTATGGACATTAG
AAAACATTGCAGTTTAGTCATAATCAAAAATAAATCTTGAGGCTGGTAG
AGCAGCTTTGTTGCTGTTTATATTTTTATTGCTTACTGGATTTCAGTGT
TACCTAGTGCCATCAGTTTGGTATTTTGCCACCTTGCACATTCAGTGAT
GTTTGATTTTTCTTTTTCCTTTTTTTCATATTACTTTTAAATCCTGAAT
AGTTTGTGGCAGCTGGAGATCACCTAGTCCACCACTGTCCAACATGGCA
ATGGTAAGTAATATTGAGTAAAGAATAGAAAATTAGTAAAATGCATGGC
TTCAGAATTATAGCAATTTGCAAAATAGGTTAATGGATGAAAATTAGAA
TGACCAGTTTAACTTTCCCCCCAGCAGATTCTTCTGTTAAACAATGCCC
CTTCAAAATAAAGGAAGAACAAGTGGGTGTTATACCTATGTTATTTGGC
TATGTTAGCACAATATGATGGACTAATTTGAGAAAAAGCATTTACTTCC
TTTACTATTACTTCTTTTCTTTATAGGGCTAAGTCTGCCTTCTGGGTCT
TTGAA
SEQ ID NO: 63
GAAGAAGCGCGAAGAGCCGTTAGTCATGCCGGTGTGGTGGCGGCGGCGG
AGACTGCGGGCCCGTAGCTGGGCTCTGCGAGGTGCAAGAAAGCCTTTGA
GGTGAAGGTGTATGAAAGTCATCATAACAGATGTTTTCCAAAAACTTGT
AGAAGGTTGTGAAAAAACTACTAGGATCACGCGGCATGTATTGAGCATA
TAGGTTGCTGTAGATGAATGTTCTTAGCTGTCATGTTTAAAAATACTTC
TGCTTCGTTACCTCAAGTGTGGCATGCAGCATTTTGGAAGGAAAATTGA
AGACGTGTTCAAGAAAACATGAACAGAAGCAAATGATGAAAATGAGCAT
TTTACTTGATGTTGATAACATCACAATAAATTATGGAGAAAAATACATA
TTTGGCTAACTTTTAATTGCTGAACAATAAAGTGTTTTCTTTTAAATCN
AAAAA
SEQ ID NO: 64
GAAGCCAAACCAAAGGGAGCTTCTACTTCATGATGCCATTTATGTAAAG
TTCAGGCAGAGAAAATCAGTGGTTTAAGAAGTTAGAATAATGATTATCT
TTGGAGGGATTGCAACTGGAAGAAGTCATGATTGGGATTTCTGGGTCCT
AATAGTGCTCTGTGTCTTGATCTGAGTGCCGACTACATGAGTGGTTAGG
TTTGCAAAATTCATTGAGTTATGCACTTAATGGTGTTGTCTTATTAGAG
CTGATGGAGGAGAGAGGGCTTCAATTTGCACAACTGAGTAATCAGCTAG
GCCCAGTCACTAGGTGAACAACTTACTGCTACCAATCAGCCTTAGAGCA
GGAATCAAACTCATGTCTCAGAAAAGTTATTAATTCAGCTTGTCTTGGG
ACTTCCTTCAGAGTCACTCTTGAATAGCTGAAATAGTAAATGTTAAATC
TGTGGATGCAAGTGTGTAAATTATTTTAGTCATCAGCTCTAATAAGATG
GCCTTTGGGGAAATGAGTATAAGGTCACGAAAATGAAATGGCAAGAAGG
AGGTCTACTATTTCTTCTGTAATACTGATTTTTACCCCATCAGGGTCAG
TCCCCAAAGGTTGTAAATGTGAAGCTTGGTCTTTTTCTTTA
SEQ ID NO: 65
GACCCTATTCTCAGGATGAAAATAATACACTAGTAATAGTCTGCTCTGT
TGGTTAACTCCTCGTAAGGAGGTACAATTAAAATGCTGTAGTGTTGCAA
GGGAAGGAGAGGAAGAATCATATTCCTTCACTAGCAGGATCAAGAAAGC
TTTTATAGAAATATACAAAATCTTCACTTCTTGAAGGATTGGTAAAATT
TAATAGCCAACATTGGGCACTTATTCATTCTCTGAGTAAATATTTATTG
CATGCTTATCTTGTATCAACATTGNGATGAAAGCNCAAGAATGAAAGAG
GAGGGAGAATGTTTANAGAATAAGGCTGAAACACAGATTTTGTAGGGAG
CGTAGGGGAGACTGANAAAACAG
SEQ ID NO: 66
AAGACACCTGATAGATTGTCTTGTATTATTTTTCCTTTGCCTTCTTACA
ATCTCAGTGATTAGAATTGGGCTGAAAACAATACATCAAATTCTCAGCA
AAATCCTTATGGGTTGCTGGATACCGAGGGTTTTTAAGATCTTTAGACT
TCACTATATAGAACAAATGTTGAATGGGAATTTTCTTTATTTCTATANC
GTTTNG
SEQ ID NO: 67
CCCGGAATCGCGGCCGCGTCGACGATGAGCATTTTTTCATGTGTCTTTT
GGCTGCATAAATGTCTTCTTTTGAGAAGTGTCGGTTCATATCCTTTGCC
CACTTTTTGATGGGGTTGTTTTTTTCTTGTAAATTTGTTTGAGTTCATT
GTAGATTCTGGATATTAGCCCTTTGTCAGATGAGTAGGTTGCGAAAATT
TTCTCCCATTTTGTAGGTTGCCTGTTCACTCTGATGGTAGTTTCATTTG
CTGTGCAGAAGCTCTTTAGTTTAATTAGATCCCATTTGTCAATTTTGGC
TTTTGTTGCCATTGCTTTTGGTGTTTTAGACTTGAAGTCCTTGCCCATG
CCTATGTCCTGAATGGTAATGCCTAGGTTTTCTTCTAGGGTTTTGATGG
TTTTAGGTCTAACGTTTCAGTCTTTAATCCATCTTTTAAAAGTCTCTTC
ACAGTACATGAGTAGTAGTGACACCAATAATGTCAGAGCAGGGAACTCC
CAGGTTCTGCCCATCCACAAAAACAACAAATAAGCTGGCAAAAACTTTA
AGAATCAACTTTTGCAGATCTCTGAAATCTAGTCAAAACTTAAACAGAG
GAAAGATTAATAAAGACNGGCTGCCTGAGATAACACTAACACACAC
SEQ ID NO: 68
CATCAAATAAATAAATAAATAAATTTTAAAAGTCACAGCATTGAATTTT
TAAATGTTTGGGATGATAAAGCACCTGCTTATCATGAAGCTANAGAAAT
TCAATGACACGTTTGCCAGGGTCTTTGCTAGTGATGTTGGAACAAGTCT
GTAATGCTGATGAAACATCACTGTTCGGGCATTATTGCCCCAGAAAGAC
ACTGACTGCAGCTGATGAAACAGCCCTTCCAAGAATTAAGGATGCCAAA
GACCAAATAACTGTGCTGAGATATACTTACGCAGCAGGCATGCATAAGT
GTAAACTTGCTGTTATAAGCAAAAGCTTGCGTTCTCACTGTTTTCAAGG
AGTGAATTTCATACCAATCCATTATTATGCTAATAAAAAGGCATGGATC
ACCAGGGACATCTTTTCAGATTGGTTTCACAAACATTTTGTACCAGCAG
CTTGTGCTTACTGCAGGGAAGCTGACTGGATGATGACTGCAAGATTTTG
TTATATCTTAACAACTGTTGTGCTCATCCTCCAGCTGAAATTCTCATCA
AAAATAATGTTTATGGCTCACACCTGTAATCTCAACACTTTGGGAGGAT
TGCCTGACCCAGGAGTTCAAGCCCACCCTGGGCAACACAGCAAGACCCA
ACCTNTC
SEQ ID NO: 69
TTTTAAAAATCATAAAACGTTTCTTACAAAAGAGCATTACATTNTGCAC
ACTGCTCTGAACAGATGCCAGGGACATGTGGACTATTGTTACTTTTCCT
CCCTGTCCCACCCCCCAAATGTTACAGTGACCACAAAGCAAGGTGTTCA
CAATAATTACATGGGGGGAATTTTTTAAACCACCAACAATAACGAAAAA
TAAAATCCACTCACTCTGCTGCTGTTTCAAAATTTCAATGTTAGTTTTT
GCACGCCCTTCCCCCCCCCAACCCTGTTTGTAAGGAACTAAAACATTAC
ATCTGGTGAACAGCAAAGATTTCACTACACCTCAAATGCAGAACACCTA
TGAAGCAGAGGAATGTTGGCTTTTTAAACAGAAGCAGATAAAAAAAAAA
GATGCAGGACTCCTTCAGTTCTTCACTAGTCTTAGAAAAACTTTCCAGA
ATACTGCTTCACACTATAAAAAAGAAAAAATATCTTGCATTAGAATCCT
TCAACATCTGCATACTGCTTCACACTGTTCGTTTCTAGGAGCACTTTGT
CACAGGACACTTCTGCTTATATTTCTTTAATCAGAACTTAGTTGGATGG
GCCGGGCATGGTGGCTCACGCCTGTAATCCCAGCACTTTGGGAGGCCGA
GG-GGGTGGATCACC
SEQ ID NO: 70
CCATCTCCAAATTTAGTATTCATTCTGTTTAGCATATTATCAGTTGCCA
TCTATTTGTTTTAACTGATTACTTGAATCTGATTAAACATCACAGAAAT
GGGCTTTGATAAGAACAATATTGAATAAGAAATTTTAAATAACAAAACA
GCTTATAGAAAAATTCAGCATAACTTTTCCATCACCTTCACCACCCTTG
CCTTTTATTATCCTGTCCTGTATCACTGCTTTCTGTTAGCAGTGTTGTG
TGAGTTAGGATTTGGGCAGGAAAGCAAAAGCAACCACCCGTCATTTTCC
CAGAATGAAGGGTTTGACGTAGGATGTAGACTTTGTATAGTAGTTGGGA
GAGCTGTGGGAGTGAAGGTCAGGGATGTCACCTACAGAAGTCAGGGAAT
CTGCCACCAGAGATCCTGCATCAGAAACAGCCAACAGCGTGCTTCTGAA
GAACTAGTGGGGAAGTGGCTATAATTCTTAGGAATCCCAGCAAGTCCGC
ACCACTGTCTCAGTCTACAGCAGTGGAGAAAGGGGTTTCCAGGAGCTCT
CTGGAAAGTTCCTGCCCACACTTTGCAACAATCTTCAGAGGATAATGGG
CTTCTCTTCCAGCTTCCACACCCAACAAGAGTGCCTTTCATCGGCCAAC
TCTAACCTGGAACCCTATGGCAGAGGGGATTTAGGAGACAGTTTGTNAT
GTCTGTGGAATGCAAATGAANANGTANCAATGCTTANTTGACAGCGGNC
ATACACAAATNTNGAAA
SEQ ID NO: 71
GATCCGTNGACT
SEQ ID NO: 72
CTCTTCCCAGCCCCTGAGCCCAGCCCCTTCCCAAGTGGTGCCAGACAAA
AAACTACATGGCCCTTTCGTGTCTTGGGGGTGGAAAGGGAGGGATGAAT
TGGGGTGATAGAACCCTGGTGAATTCAGAGTAATCTTTCTTTAGAAAAC
TGGTGTTTTCTAAAGAAACAGGATAGGAGTTTAGAGAAGGCACCAAAGC
TTTCACTTTGGTTTGGCACCAGTTTCTAACCATCTGTTTTTTCTACCCT
AGCTATCTTTTATTGGTAAAATATAAATGTATAATTATGTTTGTAGAGC
TTTACCAAGGAGTTTCCCTCCTTTTTTGTTTGTTGATTAGCAAATTTTT
GATTCTCCATTTTCCAAAAGTAAGAGACTCCAGCATGGCCTTCTGTTTG
CCCCGCAGTAAAGTAACTTCCATATAAAATGGTATTTGAAAGTGAGAGT
TCATGACAACAGACCGTTTTCCATTTCATCTGTATTTTATCTCCGTGAC
TCCACTTGTGGGTTT
nt: 505
SEQ ID NO: 73
TGGAGCTGAAAAATTCCTATTACCTAGGGGCATCACAACGCATTGCATT
TCGCCCGTGTTTGGGATGATGCTGGTGTAAACCTACTATGCTGCCAGTC
ATGTAAAAGTATAGCACACACAATTAGTAGGTAATGCTTGCAAATAATA
ATGAAAGACTCTGCTACTGGTTTATGTATTTACTATGCTATACTTTTTG
TCATTACTTTAGAGTGTACTCCTACTTTTTTTTTTTTTTTTTTTGAGAT
GGAGTTTCACTCTTGTCCTGTAGGCTGGAGCGAANTGGCGCGATCTCGG
CTTACTGCAACCTCCACCTCCTGGGTTCAAGCGATTCTCCTGCCTCANC
TTCCCAGAGTAGCTGAGATTACAGGCATGCACCGCCACGCACGGGTAAT
TTTGTATTTTTGGTAGAGACAGGGTTTCACCATGTTGGCCAGGCTGGTC
ACCAACTCCTGACCTCAGGTGACCCGCCTCCTCACCTCCAGAGTGTTGG
GATTACAGGNGTGAG
SEQ ID NO: 74
ATAAAAATTAGCTGGGGGTGATGGGCCCTGTACCCCAGCTACTCGGGAG
GTGAGGTAGGAGAATCACTTGAACCCGGGAGATGGAGGTTGCAGTGAGC
CAAGATCGTGCCACTGCACTCCAGCCTGTGTGACAGAACAAGACTCTGT
CTCAAAAAAAAATAATAATAATAATAATAATAAAAAGGAATAACATAGC
TAGGAATAAATTTAATCAAAGAGGTGAAAGACTTATACACTTAAAACTA
CAAAAAAAAAATCACTGAAGGAATTATAGACCCAAATAAAAATAAATAA
AAAGACATTCTGTGTTTTAGGGAAAGAAGACTTAATATTGTTAAGATGT
CAATACTACCCAAAGTGATCTACAGATTCAACATAATCCCTATCAAAAT
TCCAACAGCCTACTTTGTAGAAATGGAAAAGCCAATTTTCAAATTCAGA
TGGAATTGCGAGGGGTTCTGAATAACAAAACACAATCTTGGGGAAAAAA
AACAAAAAACAAAGTCAAAGAACTCACACTTCTCTATTTATAATTTACT
ACAAAGTTATAGNATCAAAGTCGACGCGCCGCGATCCGGGC
SEQ ID NO: 75
CACAGTACTCCATTTTGGGGTCCAAACTGTAATGCTCAAAATAATAAAT
GCTTACACGAAAATTATTTATTGAGAATATTCATATAAAAATTACCTAA
AGCAAAGTAAAAAAAGTAAAATCAAGGTGGTATATTTGAAGTGAATGGT
GATTGGAAATTTTTAGCTGTAACAAAAAGAAAGAAAACAACTTTTTTTA
AAGCCTCATTCTCTTTTCTTTCAAAATGTACCTTATTCCCACACACTCT
TGGGCTGACCTTTATTTTATCAATAAGCTCAATATTACTTTGTTTAAAA
TAAGATGCTTCAGCAAAAGTCATTCTCTCTTTAACCATATAATTTAAAA
ACTCCTCTTCACGATTGATAGCAAAATCAGAAACGTTAGGGCACCAGTG
AGTTGAAAAAACTGGTCTTAAGTTGGAAAAACTATTATTAATAATATTA
TCCTATCCATCCATATCTATTGAAATTGTCAGGTCCATAATTTCATTTT
AATTAATTATAGGAAAGAAGAAAAGATAATACCCATTTGTTCTAT
SEEQ ID NO: 76
CTCAGACTCTTTCTGCCCTAATGGCCATTACTATCCAGTCTGTATTGCT
ACAAGGGACCCACTGGTACCCCTTTTAGATTCTATCAAAAGGAACAGGG
TTTTCCTAGAGGCAGGCAGCCTGGTGGTATGGCACAGCAGAAGCTTACT
GCTAATGAAATGGGAACCTCCCCCTCCCTTGTGGTTTCAGCACAGAACC
TGAATGCCAGGAAAAATTCCTGGGCCAAGAAGCTAAAGCTAAAGAAACC
TTCCTTTTTTCAACGTTTTTTTTTCTTTCAAACTGTAGGGTCACTTTTG
ATTGAGGCAAAGGGGTCCTACTGTAAGTGGAAAAGACTCACTCCCCTAA
CATAAGTTTTCACTGTGGTGGGATGGTGCCGCCCGATATGCTTGATATG
CTTTTCCTTCCACATGTTAAGCTAGGAAACCTAACAGGATGTCAGCAGG
GCAGTTAACTCTGGACTCANAGCCCTCAAGGGCATGTGGCANAACCTCA
TGGCATNCAAGACCA
nt: 596
SEQ ID NO: 77
GTATAATTGATTCTTTTGAACCTAAAGTATAAGACTTCACGATTAGAAA
AAAATTATCCAAAGACTAATGTAATTAAGTGAGGAAAAGGTGCTGGAGG
AACTGGATAACCACATGGAAATGTATGAACCATGACCTCTATGTCACAT
ACTATATATAAAACTTAATTTGAGGTGTATCACAGAGCTAACTGTGGGG
GCTAAAACGTTGAAGCCTTTGGATGGCCGCACAAGAGATGTCTGCATTC
ATAACCTTGGGGAGGGTATGAACATTTCTTGGTAACATGCAAAAAGCAC
TAACTGTAAAAGAGAACAGTTGGTCAGTTGAATTTCATGAAACATTGTA
AACTTCTGCTAAACAACTGACACCATTAAGAATGTGGAAAAAGGCTGGG
CACAGTGGCTCATGCCTATAATCCCAGCATTTTGGGAGGCCGGGGCGGG
AGAATCACTTGAGGCCAGGAGTTTGAAACCAGCCTGGGCAACATGGCAA
GACCCCGACTCTACAAAAATATTTTTAAAAATTAGTTGGGTGTGGTGAT
GCACTCCTGTAGTCCTAGCTGCCAGGANGCTAAGGNGGAAGGATCACTT
AACCCTGG
SEQ ID NO: 78
CTGGTGGCGGCGGTCGTGCGGACGCAAACATGCAGATCTTTGTGAAGAC
CCTCACTGGCAAAACCATCACCCTTGAGGTCGAGCCCAGTGACACCATT
GAGAATGTCAAAGCCAAAATTCAAGACAAGGAGGGTATCCCACCTGACC
AGCAGCGTCTGATATTTGCCGGCAAACAGCTGGAGGATGGCCGCACTCT
CTCAGACTACAACATCCAGAAAGAGTCCACCCTGCACCTGGTGTTGCGC
CTGCGAGGTGGCATTATTGAGCCTTCTCTCCGCCAGCTTGCCCAGAAAT
ACAACTGCGACAAGATGATCTGCCGCAAGTGCTATGCTCGCCTTCACCC
TCGTGCTGTCAACTGCCGCAAGAAGAAGTGTGGTCACACCAACAACCTG
CGTCCCAAGAAGAAGGTCAAATAAGGTTGTTCTTTCCTTGAAGGGCAGC
CTCCTGCCCAGGCCCCGTGGCCCTGGAGCCTCAATAAAGTGTCCCTTTC
ATTGACTGGAGCAG
SEQ ID NO: 79
GCAGGGGCTTCTGCTGAGGGGGCAGGCGGAGCTTGAGGAAACCCGCAGA
TAAGTTTTTTTCTCTTTGAAAGATAGAGATTAATACAACTACTTAAAAA
ATATAGTCAATAGGTTACTAAGATATTGCTTAGCGTTAAGTTTTTAACG
TAATTTTAATAGCTTAAGATTTTAAGAGAAAATATGAAGACTTAGAAGA
GTAGCATGAGGAAGGAAAAGATAAAAGGTTTCTAAAACATGACGGAGGT
TGAGATGAAGCTTCTTCATGGAGTAAAAAATGTATTTAAAAGAAAATTG
AGAGAAAGGACTACAGAGCCCCGAATTAATACCAATAGAAGGGCAATGC
TTTTAGATTAAAATGAAGGTGACTTAAACAGCTTAAAGTTTAGTTTAAA
AGTTGTAGGTGATTAAAATAATTTGAAGGCGATCTTTTAAAAAGAGATT
AAACCGAAGGTGATTAAAAGACCTTGAAATCCATGACGCAGGGAGAATT
GCGTCATTTAAAGCCTAGTTAACGCATTTACTAAACGCAGACCAAAATG
GAAAGATTAATTGGGAGTGGTAGGA
SEQ ID NO: 80
CCCGGAATCGCGGCCGCGTCGACGGGAGGTGATAGCATTGCTTTCGTGT
AAATTATGTAATGCAAAATTTTTTTAATCTTCGCCTTAATACTTTTTTA
TTTTGTTTTATTTTGAATGATGAGCCTTCGTGCCCCCCCTTCCCCCTTT
TTTGTCCCCCAACTTGAGATGTATGAAGGCTTTTGGTCTCCCTGGGAGT
GGGTGGAGGCAGCCAGGGCTTACCTGTACACTGACTTGAGACCAGTTGA
ATAAAAGTGCACACCTTATAAAAAA
SEQ ID NO: 81
CCCGGAATCGCGGCCGCGTCGACGGGAGGTGATAGCATTGCTTTCGTGT
AAATTATGTAATGCAAAATTTTTTTAATCTTCGCCTTAATACTTTTTTA
TTTTGTTTTATTTTGAATGATGAGCCTTCGTGCCCCCCCTTCCCCCTTT
TTTGTCCCCCAACTTGAGATGTATGAAGGCTTTTGGTCTCCCTGGGAGT
GGGTGGAGGCAGCCAGGGCTTACCTGTACACTGACTTGAGACCAGTTGA
ATAAAAGTGCACACCTTATAAAA
SEQ ID NO: 82
CTTCATTTGAAATGGTTGAATCTGCTGTGTAATAAAGTGGTTCAACCAT
GATTAGGAACTGAAATTTAGTAGAAGAGGGAAAAGGAGTTAATGTAACA
AATTATTTTAGCTACAAACCCCGGTAATAGAGCACTTGGGGGATGGGAT
GGGGTGGGTTGGTGAGACAATCAGAATGGTAAATTGATTAAATGCTCCT
AACCCTGTAATTTTGTGCATAGAGCACCCTATGCTGTGGAAATAACTGT
TCTTAGATTTCATTGTAACTGGACTGTTCAGGTTGCCCAGAGGGAAAGA
ACATTCCTAATTCTAATAAAATAAACTTTTATTTTGTTTA
SEEQ ID NO: 83
TGTCATTGAATCTGCTTGTTACTTAAATGCTAAACTCAATTCTGTAATT
CAATAGGTGCACCTCTCTGAGAAACATAAGAGACAATGAGGAAAAGGAT
TCAGCATTCCGTGGAATTTGTACCATGATCAGTGTGAATCCCAGTGGCG
TAATCCAAGTAAGATGTTCACAAAGATTTGTTTTTAATGTCTAATTAAT
AAAATTTTAAAGGAAGAAACATTCTAATACTTTAATTATAAAAAGTTAA
CTATTTTCAAAGGTATCAAAATACAGTTAAACCTTTAAAATGTATATTT
CTTAATATCTTGAAATTGTAATGCCTTTTTTTTTTCCTAAATTTTTTTT
GTCATGAAATGAGATAGTAACAGCAGATTGGGACAACAAGGTTATATTC
TTGTCTTGAATCAGGCCATGGCTTCTTTCATCCAAATTTCAGACCTCAT
TTATTTACTTTGTCCCTGCCTCCCATCCCTGGATATCAGTTTGTGGATA
TCTACAGTTAATAGAGTGACCAAATAGTAGGAATACTGTCTCTCTATTC
TGAATAAAATCTTTGAATCAGATTTAGAAATAATGAATAAAATACAAAT
CAGCCATTGAAATTGCTCTAATTTTGAGAGCTTATGATTTATTCATCTT
TGGTTTCCAAGTTCAAGTTATATGTAGACATTTTA
ATT
SEQ ID NO: 84
GCTTCCTAGGTGAGGTCACGAGGAAACCTGCTGGCCAAGTGACCTGGCA
GGGTGTGGCCAGTGTGGCCAGGGCCGCCGAGCCTGCTTTCCTTCCCTGC
AGCAGGAACCCTTCTGGGGCTGTGATCCTGCGATGGTGCCTGGGTGGGA
GTGGGGGTGGGGGGCGGGATGGTCTCCCTACCTGCCAGCTTCTTGGTTT
GAGGTGAGGACAGCCCCGGAAGCTCANACTTGGCTCCTGTCCATGTACT
TGGGGCCATGAGCTCTGCAGGGACCTTGGAAAGANAGAGACGGGTGGTG
TANGGCANGGGAAGGCATTGTCTTCAAACAGGAAAAAGCTGANAATGGA
AACAGGCGAAACTTACCAAGTGTAACATCACCTGGAACTGAAGGAGGGT
GGGAAGGTTTTAATTATTTTAAAAATAGAGATGGGGTCTCACTATGTTG
CCCAGGCTGGTCTCAAACTACTGGGCTCAAGTGAACCTCCTTCT
nt: 387
SEQ ID NO: 85
TGTTTCTCNAGGGCGAGAGGCTGTCTTANAGCACCATTCTCTGGCCCTN
GTCCCATGAGAAGGAACCGCACTCAGGAGCCACACTCTCCCACTNCCCT
TGCCCANAAGACTCACAGAGGGCACGGAGCTGGCTGTGGTGAGAGGAGG
TCCANCAAATTCCTGTCTGCANAAGGGTTCTGAACACCACCGCCTGGCA
GCGTGCTGGAGGAGGGATTCCTCTTTTCCTCACAGCAATTCTGACCAGA
AACCTGTCAAATCAGGAATGGCTAAAATAAGACCAGGGTATGAATGACC
ATCAGCCACAGTAAAACCAAGGCACAGCTCTCCTGAGCCCACCCAAGCT
GCTGTGGCCCAGACTGGTGACATCACCTCAGGGCAAAAAAAAAA
nt: 420
SEQ ID NO: 86
CGCAGAATGGCTCCCGCAAAGAAGGGTGGCGAGAAGAAAAAGGGCCGTT
CTGCCATCAACGAAGTGGTAACCCGAGAATACACCATCAACATTCACAA
GCGCATCCATGGAGTGGGCTTCAAGAAGCGTGCACCTCGGGCACTCAAA
GAGATTCGGAAATTTGCCATGAAGGAGATGGGAACTCCAGATGTGCGCA
TTGACACCAGGCTCAACAAAGCTGTCTGGGCCAAAGGAATAAGGAATGT
GCCATACCGAATCCGTGTGCGGCTGTCCAGAAAACGTAATGAGGATGAA
GATTCACCAAATAAGCTATATACTTTGGTTACCTATGTACCTGTTACCA
CTTTCAAAAATCTACAGACAGTCAATGTGGATGAGAACTAATCGCTGAT
CGTCAGATCAAATAAAGTTATAAAATTG
SEQ ID NO: 87
GGAAACTGATGCCAGTCAGAAACTCAGATCAAATGAAGGGGTGAAGAGA
ACCAGAATTGATCTCTCTGTAGGAGAATATAAATGACTTTTTTAAAGTA
CATATTTTCTGTGAAAGACAGTTTTTTGTTTAATGCAAAAATGTTAACA
ATGTTTATATCATGTAGAAGTAAAAGATCGTGAAACAGCACAGAGAACA
GTAGTAAGACAGATTGAATTGCACTGTTGTAAGATGATGAACTTACAAT
ATTAAGTGAAGGTAGACTGTGATAGATTAAGGATATATATTGTAATCCC
TAGAGCAATTGTCAAAGTGGTACAGGTAAAAAGCCAATAGAGGTGATAA
AATGGAATACTAAAAAATATCAGATGAATAATAAAGAAGACAGGAAATG
AGGAACAGTGGAACAGAATGAATAAAAAACAAGACCATTAACTTAATCA
TTAATAATTACTTTAAATGGGTTAAACATTATGGTTATAAGGCAGAGAT
TTTCAGACTAGATAAAAGAGCAAGCTCCACTATATACTGTCTACAAGAG
ATATACTTTAAAGTGTATATTATATTTAAATATAAAGATTTGGAATAAA
TAAACCTAAGAATAAGCTTACTAGGGAAGTGAAAGATCTGTACAACAAG
AATTACAAAACACTGCTGAACGAAATCATAGGTGA
CCA
SEQ ID NO: 88
GTCCCGGAATCGCGGCCGCGTCGACGTTTCCTCAAAATTTATCTTCCTG
TTAATGTCAGGCATGTATCTCCTTAGCTTGCCACAAATAACTATATATA
CCACAGACCTTCCTTTGTAGGGCTAACAGTGTTGCATTGTAAGTGGAGG
CCTCATAGATACCTGGCCTTTTCCTACCTTATTCCAAAGATGGTTGCAT
CTTATAAATAATGTCATTCTTCAGCAAATGGTATGGAAATGAGATTGTA
ATGTCATTATTTCCTCTTTAAATAATCAGGACAACTCATGATACAAAGA
GCTCTTCTCTATAAAAGGTGGGACTTTTTTTTTTAGTAATAGCAAAAAT
AAAATTGTACCTCCTTAATCTTCTACAGAAAGATGGATTTCATTTTCAA
CATTAAGAGGTAGTTTTAAGAAGCAGTAGAAGTCAGCCTGGGCAGCATG
GTGAAACCCCGTCTCTACAAAAAAGTTAGCTGGGCTTAGTAGTTGCAAT
CCCAGCTACTCTGGAGGCTGAGGTTGGAGATCATCTGANCCTGGGGAGG
TCNAGGCTGCAATGATACANTGAGCCCTGATTGTGCCACTCCACCTGGT
TGCAGA
SEQ ID NO: 89
TTCCAATCTTCGTGTTCACTTTAAGAACACTCGTGAAACTGCTCAGGCC
ATCAAGGGTATGCATATACGAAAAGCCACGAAGTATCTGAAAGATGTCA
CTTTACAGAAACAGTGTGTACCATTCCGACGTTACAATGGTGGAGTTGG
CAGGTGTGCGCAGGCCAAGCAATGGGGCTGGACACAAGGTCGGTGGCCC
AAAAAGAGTGCTGAATTTTTGCTGCACATGCTTAAAAACGCAGAGAGTA
ATGCTGAACTTAAGGGTTTAGATGTAGATTCTCTGGTCATTGAGCATAT
CCAAGTGAACAAAGCACCTAAGATGCGCCGCCGGACCTACAGAGCTCAT
GGTCGGATTAACCCATACATGAGCTCTCCCTGCCACATTGAGATGATCC
TTACGGAAAAGGAACAGATTGTTCCTAAACCAGAAGAGGAGGTTGCCCA
GAAGAAAAAGATATCCCAGAAGAAACTGAAGAAACAAAAACTTATGGCA
CGGGAGTAAATTCAGCATTAAAATAAATGTAATTAAAAGG
SEQ ID NO: 90
TGCAGGATCCGTCGACTCTAGATAACATGGCTAGAAAAGAGAATGAAAA
AGTTGGAATTTTTAATTGCCATGGTATGGGGGGTAATCAGGTTTTCTCT
TATACTGCCAACAAAGAAATTAGAACAGATGACCTTTGCTTGGATGTTT
CCAAACTTAATGGCCCAGTTACAATGCTCAAATGCCACCACCTAAAAGG
CAACCAACTCTGGGAGTATGACCCAGTGAAATTAACCCTGCAGCATGTG
AACAGTAATCAGTGCCTGGATAAAGCCACAGAAGAGGATAGCCAGGTGC
CCAGCATTAGAGACTGCAATGGAAGTCGGTCCCAGCAGTGGCTTCTTCG
AAACGTCACCCTGCCAGAAATATTCTGAGACCAAATTT
nt: 535
SEQ ID NO: 91
CACAGTACTCCATTTTGGGGTCCAAACTGTAATGCTCAAAATAATAAAT
GCTTACACGAAAATTATTTATTGAGAATATTCATATAAAAATTACCTAA
AGCAAAGTAAAAAAAGTAAAATCAAGGTGGTATATTTGAAGTGAATGGT
GATTGGAAATTTTTAGCTGTAACAAAAAGAAAGAAAACAACTTTTTTTA
AAGCCTCATTCTCTTTTCTTTCAAAATGTACCTTATTCCCACACACTCT
TGGGCTGACCTTTATTTTATCAATAAGCTCAATATTACTTTGTTTAAAA
TAAGATGCTTCAGCAAAAGTCATTCTCTCTTTAACCATATAATTTAAAA
ACTCCTCTTCACGATTGATAGCAAAATCAGAAACGTTAGGGCACCAGTG
AGTTGAAAAAACTGGTCTTAAGTTGGAAAAACTATTATTAATAATATTA
TCCTATCCATCCATATCTATTGAAATTGTCAGGTCCATAATTTCATTTT
AATTAATTATAGGAAAGAAGAAAAGATAATACCCATTTGTTCTAT
SEQ ID NO: 92
CAGGGGCTTCTGCTGAGGGGGCAGGCGGAGCTTGAGGAAACCGCAGATA
AGTTTTTTTCTCTTTGAAAGATAGAGATTAATACAACTACTTAAAAAAT
ATAGTCAATAGGTTACTAAGATATTGCTTAGCGTTAAGTTTTTAACGTA
ATTTTAATAGCTTAAGATTTTAAGAGAAAATATGAAGACTTAGAAGAGT
AGCATGAGGAAGGAAAAGATAAAAGGTTTCTAAAACATGACGGAGGTTG
AGATGAAGCTTCTTCATGGAGTAAAAAATGTATTTAAAAGAAAATTGAG
AGAAAGGACTACAGAGCCCCGAATTAATACCAATAGAAGGGCAATGCTT
TTAGATTAAAATGAAGGTGACTTAAACAGCTTAAAGTTTAGTTTAAAAG
TTGTAGGTGATTAAAATAATTTGAAGGCGATCTTTTAAAAAGAGATTAA
ACCGAAGGTGATTAAAAGACCTTGAAATCCATGACGCAGGGAGAATTGC
GTCATTTAAAGCCTAGTTAACGCATTTACTAAACGCAGACGAAAATGGA
AAGATTAATTGGGAGTGGTAGGATGAAACAATTTGGAGAAGATAGAAGT
TTGAAGTGGAAAACTGGAAGACAGAAGTACC
SEQ ID NO: 93
CGCTGGGTGCCTGCAGCGCCTCCCTTGTCTCATATGGTGTGTCCAGCAC
TCTATTGTTGTAAACTGTTGNTTTGNCTGACCTAAATTNTCTTTACTAA
ACANATTTAATAGTTNAAAAAAAAAAAANANCA
SEQ ID NO: 94
TTTTAAAGTCATCTCTATAGGAAGGTGCTGGGCAGGGATCCCAGAGAAA
GAAAGGGTCCAAGACTCCATTAACTGCCCTGGATGAAGGGCACTGCTAC
AGCAGCTAGTACCAGAGACTCTCCTATCTCACGGTTGAGGCAGACCCAG
GATAGAATAGAGAATAAAAGGAATGCTTATAGGAAACAATTTTGTATGG
AATGCTAGATGGCCAAGCCTCAGCCTTTGGTCCAGTGCAACCCTTGCCT
CGCTTGTCAACAGTGAAAAATTAGTTTGGTTAGAAGAACCATCTGGAAA
CACACCAGCTTCTGCTACCTTCATGCTCATTGTTAAAAAAAGATTAACC
AGTGTGAACATTCTGATCTGTTAATTCCAGGGACTGTTTTCTTTCCAAT
GGACTGTTTGTTGGTAGAATAACCCCCAAAAGCTCAAAGCTAAAATGCA
TCATCAGTCCTAGTCGGCAGTTCCTTAAGAATGGACTGGCGGCGTGGTT
GAGCTGATATGGAAAAGCTGCACCTTCCTGCAGAAGATCAACTGACCTG
CTATCCCACCCCAAATTTCAACCTGAGGTATATTTCAATGAAGGCAGGT
AGCTGTGCTTCTCAGAGCA
SEQ ID NO: 95
TCCCGGAATCGCGGCCGCGTCGACCCGCCGCCGAGGATTCAGCAGCCTCC
CCCTTGAGCCCCCTCGCTTCCCGACGTTCCGTTCCCCCCTGCCCGCCTTC
TCCCGCCACCGCCGCCGCCGCCTTCCGCAGGCCGTTTCCACCGAGGAAAA
GGAATCGTATCGTATGTCCGCTATCCAGAACCTCCACTCTTTCGACCCCT
TTGCTGATGCAAGTAAGGGTGATGACCTGCTTCCTGCTGGCACTGAGGAT
TATATCCATATAAGAATTCAACAGAGAAACGGCAGGAAGACCCTTACTAC
TGTCCAAGGGATCGCTGATGATTACGATAAAAAGAAACTAGTGAAGGCGT
TTAAGAAAAAGTTTGCCTGCAATGGTACTGTAATTGAGCATCCGGAATAT
GGAGAAGTAATTCAGCTACAGGGTGACCAACGCAAGAACATATGCCAGTT
CCTCGTAGAGATTGGACTGGCTAAGGACGATCAGCTGAAGGTTCATGGGT
TTTAAGTGCTTGTGGCTCACTGAAGCTTAAGTGAGGATTTCCTTGCAATG
AGTAGAATTTCCCTTCCTCCCTTGTCACAGGTTTAAAAACCTCACAGCTT
GTATAATGTAACCATTTGGGGTCCGCTTTTAACTTGGACTAGTGTAACTN
CTTCATGCAATAAACTGAAAAGACCATGCTGCTANTC
SEQ ID NO: 96
TTCGGACGCAAGAAGACAGCGACAGCTGTGGCGCACTGCAAACGCGGCAA
TGGTCTCATCAAGGTGAACGGGCGGCCCCTGGAGATGATTGAGCCGCGCA
CGCTACAGTACAAGCTGCTGGAGCCAGTTCTGCTTCTCGGCAAGGAGCGA
TTTGCTGGTGTAGACATCCGTGTCCGTGTAAAGGGTGGTGGTCACGTGGC
CCANATTTATGCTATCCGTCAGTCCATCTCCAAAGCCCTGGTGGCCTATT
ACCANAAATATGTGGATGAGGCTTCCAAGAAGGAGATCAAAGACATCCTC
ATCCAGTATGACCGGACCCTGCTGGTAGCTGACCCTCGTCGCTGCGAGTC
CAAAAAGTTTGGAGGCCCTGGTGCCCGCGCTCGCTACCAGAAATCCTACC
GATAAGCCCATCGTGACTCAAAACTCACTTGTATAATAAACAGTTTTTGA
GGGATTTTAAAA
SEQ ID NO: 97
CTGCAATGTGCAATAGTTGCACCACTGCACTCCAGCCTGGGTGACAGAGT
GAGAACCTATCTCTTAAAAAAAAAAAAAAAAAAAGGAAGAAGAGACATGA
GAGGGCCCAAGTCACTTGCTCACTCACTTTCCGTGTACATGTACCAAGAA
AAGGCCATGTGGGAAAGAGCAAGAAGGCAGCCGCCTTCAAGACAGGAAGA
GAGCCCTCACCAGAAACTGAGCCAGAACCTTGGAATTCCAGCCTCCANAA
CTGTGAGAAAAGAATTTTCTGTTGTTTCAGTCCCCCACACTATGGCATTT
TGTTACGGCAGCCTGAGCTAATACTCCTACTTTGTCCTGCATTTACTTGG
TCTTCCAGTTAGTTTTTTAGACTTTGGGAATCAGAGCAGTCAGTTGTCAG
ATTTTAGCTTACAGTTGTCCTACCTGTGCAACTGAAATTTCTTCCATTTT
AAACCAGAGCAGAGTTTTAGAGTCAAAAGAAACCAGATCTTTTAGTGCAG
AAGCTTTCCACTGTATTANAAGTGAGGAAGTTGGT
SEQ ID NO: 98
AAAAAAACTCCAGAGAAGTTTATAGAAAGAGATGACATGTAAACCCTGCT
GAAAAATAGTTTCATTTGTTAGAATATAATTGTCTTCCACTAAAAAAAGA
AAAAAAAAAGCATTTAAGGCTCTAAGATCTCTTGAAGTACCACTTTTCCT
GAATCCCAGAGTTTTTATGTGCATTATTTTTATGCGTTTGTAGTTTGATA
TGTTGTATTTATAAGTAGTTTTAGCTTTCCATTATGAATTCTTCTTTGAC
CCATGAGTTATTTAGGTAAGTGTTTAAAAATTTACAATAGTTTATATATG
CAAATATTATGTTGTTAGAGTTGGTTTTCATGTCATTTTTACATATACAG
GGGCAGTTTCCCCAACTAAATTGTATATTCCTTAAAGCAGCACTCTTAAA
TTTTATTTCTGTGTCAATTTCTTGNCTGTGTTTCCTGGCATGGAATACAT
GGCATAAAATTTGTTATGTAATTAAATGAAATATTATTATACTTTCTATT
TTTTAGAAAAAA
nt: 577
SEQ ID NO: 99
GTCGACAGGGATGACATAACTATTAGTGGCAGGTTAGTTGTTGGTCACTT
TCAACTCTGGGTTCAAGCGATTCTCCTACCTCAGCCTCCCGAGTAGCTGG
GATTACAGGCATGCACCGCCACACCTAATTTTCTATTCTTAGTAGAGACG
GGGTTTCTCCCTGTTGGTCAGGCTGGTCTCGAACTCCCGACCTCAGGTGA
TCTGCCTGCCTCAGTCTCCCAAAGTCCTGGAACCACAGACATGAGCCACC
ACGCCTGGCCCCTTTTAAAATATTTCTGCTCATTGATGATGCACCCAGTC
ACCCAAGTGCTCTGATGGAGATGTATAAGGAGATGAATGCTGTTTTCATG
GCTGCTAATACAACATTCATTCTGCAACCCCCAAATCAAGAAGTAATTTT
GACTTTCAAGTCTTATTATTTAAGAAATATATTTTGCAAGACTATAGCTG
CCATAGACCGTGATTCCTCTGATGGATCAGACAAACTAAAATGAAAACCT
CCTGCAACGTATTCATCATTCTAGATCCCTGAGGAATCGCCACACTGACT
TNCACAATGGGTGAACTGGGTTACAGT
nt: 552
SEQ ID NO: 100
AAACAAAATTATTCTCTGAGAGGGAAAGGACATTTGAGGGAAACATCAAA
TTTCCCCATAAATAAATGAATGGAGTTTGCAGGAAGGTGAGGGTGAGCAG
AGATGTGTGTGGACATCTCTGACCATCCATCGCTGTATTCAAATGGATTG
TTTTATTCCATTCTGGTCTCAGGCATGACCACGTCCAGTGAAGACATTTG
AGGCAGCACATCTCAGGACCCAGGCAATAGACTGGCCCCAACTCAGGCTG
GACTAAGGTGTGATTAATTCTTTGTTTTTTGTGTGGAACAGCTCACCTTG
TCAGACAGCCTCAGGGCATCTCTGAGACACAGGGGCAGAAAATGACATTC
ATCTTTTGAGTCCTCATCCATGGAGTGCTGTGTTTGGGGGGCTGCATCTG
CTGAAGCGAGAACCCCATTCTGCCACCCCACCAGGATGCCCATTCTCCAG
GACTTCTCCAACTTACTATTAGACTAAACCAGAACAAGCAACAAACTGTA
TTTATGCAAGCAAAATTGATGAGAAAATTATATTCAAATAAAGCAAAAAT
TA
nt: 606
SEQ ID NO: 101
TCGTGCCACTGCACTCCAGCCTGGACGACAGAGTGAGACTCCATCTCAAA
ATAAATAAATAAATAAATAAATAAATAAATAAATAAAAAAATAAAAAATA
CTTCTGCTATGAAAAACCTAGTTGGTATTTTTGCTTATTTAATACTATAG
AAATATGGTGATCTCATCTTTAATAGAGTGCTTTTAAGGTCCCCAGTGAT
AATCTCCTAAAATCATGAACTTTAAGAATTTATAATGTTAATATGAGGAA
ATGAAATCTGGATTATCTCACCACATATTATATAATTCATTAGTGACAGA
GCAAGAACTCCAGGTCACCTGTCTATTCCATGTTTTTCCTATCTGCCTTT
AAATGTTGAGATACTACCCTTATCTCATGTGAATGGAGAAACTGCCTAAA
ATGCTAAAACTGACTCAGAGGCACCCAGACATAAGTGAAGTGTGATTAGA
AAATCCTGGTCAGTTGAGTCTTAGCCAAATGTGTACCTACTGTGTCTGCC
TCTATCAAGTCAATGAAAACATGATCTGAGAACTGTAAGTCCATTTATGG
AAAGGGTTGATTTANAGATATTTTGAACTTNCAGTGATGAGCCCCTTCTC
AAATAG
SEQ ID NO: 102
CGGACTCCTGTGCTAATTGTCAGCTTACATATCATTGTATAGAGACTGTT
TATTCTGTACCAAACTGATTTCAAAAGTACTACATNGAAAATAAACCGGT
GACTGTTTTTCTTCATAAAGTTCTGCGTTTGGCATCTTCACTCTTTCCAA
AATGTATCTGTACATCANAAATGTCACTATTCCAAGTGTCTTTTTAGTGT
GGCTTTAGTATGGCTTCCTTTTAATATTGNACATACATTGNATCTTTGTT
TTATGGNAATAAGTAATAAAAATGTAGACTTCATATTTTGTACAAAATGT
CCTATGTACAGAATAAAAAAGTTCATAGAAACAGCCNANAA
SEQ ID NO: 103
AGGCCGAGGCAGGCAGATCNCNTGAGGTCAAGAGTTTGAGACCAGCNTAG
CTAACATGGTGAAACCCCATCTCTACAAAAATATA-AAAATTAGCCTGG-
GTGGTGATGGGCACCTGTAACCCCAGCTACTCGGGAGGCTGAGGTAGGAG
AATCACTTGAACCCGGGAGATGGAGGTTGCAGTGAGCCAAGATCGTGCCA
CTGCACTCCAGCCTGTGTGACAGAACAAGACTCTGTCTCAAAAAAAAATA
ATAATAATAATAATAATAAAAAGGAATAACATAGCTAGGAATAAATTTAA
TCAAAGAGGTGAAAGACTTATACACTTAAAACTACAAAAAAAAAATCACT
GAAGGAATTATAGACCCAAATAAAAATAAATAAAAAGACATTCTGTGTTT
TAGGGAAAGAAGACTTAATATTGTTAAGATGTCAATACTACCCAAAGTGA
TCTACAGATTCAACATAATCCCTATCAAAATTCCAACAGCCTACTTTGTA
GAAATGGAAAAGCCAATTTTCAAATTCAGATGGAATTGCGAGGGGTTNTG
AATAACAAAACACNATCTTGGGGAAAAAAAACAAAAAACAAAGTCAAAGA
ACTCACACTTCTNTATTTATAAATTTACTACAAAGTTATAGTAATCNAA
nt: 329
SEQ ID NO: 104
TACGCACACGAGAACATGCCTCTCGCAAAGGATCTCCTTCATCCCTCTCC
AGAAGAGGAGAAGAGGAAACACAAGAAGAAACGCCTGGTGCAGAGCCCCA
ATTCCTACTTCATGGATGTGAAATGCCCAGGATGCTATAAAATCACCACG
GTCTTTAGCCATGCACAAACGGTAGTTTTGTGTGTTGGCTGCTCCACTGT
CCTCTGCCAGCCTACAGGAGGAAAAGCAAGGCTTACAGAAGGATGTTCCT
TCAGGAGGAAGCAGCACTAAAAGCACTCTGAGTCAAGATGAGTGGGAAAC
CATCTCAATAAACACATTTTGGGTTAAAA
SEQ ID NO: 105
GAGCAGTGGCATGATCACACCTTACTGCGGCCTCCAACCCCTGAGCTTAA
GTGATTCTCCCGCATTATCCTCCTGAGTAGCTGAGACTACAGGTGCATGC
CACCATACACTACTAAATTTGGGTCGGGTGGTGGTGGTGATTTTTTAATA
TTTTTGTAGAGACAGGGTCTCACTGTGATGCCCAGGCTGGTCTTGAACTC
CTGGGCTCAAGCAGTCACCCACCTCAGCCTCCCAAAGCACTGGGATTACA
GGTGTGAGCCACCACACTGGCCAGCTTTGTTTTGTTTTGATGACTAAGCT
GCTCTTGCTAAAAGGGCTTCTCTCTGAACTTCCCTACCTTTCTTCTGTTT
CCCTGGGCTAGGGCTCCATGTTGGCAGTCCTACTCCCAATTAACCTGGGG
CTGTCTGGTTAACCTTTATAAGATCTGCAGTCATTGGGAGACCCGGGGAC
CAGGAATATTGTTGTTGAGGGAGCTACCCTGGAAAGTGGATGGGTGGCCA
AAGG
SEQ ID NO: 106
TTTGGCTTTGCCTCTAGGCATTAGATGTTATCTTTGGAGGCATCCTTCTA
TGAGCATTCATTTTTGGACCAAGCCTGGATTTACAATTCTATTACTGGCC
CAGACTTCATTTCTATCCAATTTCATTCCACTGTGCTATAGTTTACAACA
TATAATTTGACTTATAAATAATTCCTGACTATGGGTTTAAAGACTGAAAA
TGGATCAATAGAAACTTTGAAAATGTTAACATCTTGATTGCTTTTCTCAG
TGTAGAAATGGACAATGTTTAGCTTAAAAACTGCATGTTTTTAATGAGAT
ACGGGGTTGAAAGACTTATTCCTGGAATTTATTGTTCTGGAGAAAGCCTG
TTGCTATCTGCCATACCTTGGTTTACTTTGTGCAAAATGAGCTTCTTTTT
AAGTAATGAGCTCTTTCCATGTTCAGCTTAAATTGCTGTCTTAGACACTT
CATCAGGGTTCCCTGCTCTGCCTCATTCCCCCTTTTGCTCACTTGCAGCC
TTTGACATAATCCTGGGAGGCAATTGGCATCATACATATTTTGCTTTGTA
ATCTCCTGCTTTGATTCTGACTGGGACCCAGC
nt: 747
SEQ ID NO: 107
GGATCTAAGACCAGCCTGGCAGCCACCAGATGGTGATTCTAGTCCTGGCT
CAGTCAGTAATAGGTCACTGACCCCAGAGAAATCAATTCAGCCTCCCCAG
GTCCTTGGATTTCTTTCTGTGAAAATGAAAGCATAGGTAGGAATTTCCCA
TGGAACAGCTAGCAGAGGAGAAATATTAAAAGTCAGGAGACTCATGCTAT
AGTTTTCATACTTCATTACAACAATGTTGTTTAGGACAAGTGAGTTAACC
TGTTAGCTTCCTCTATATAAAATGGAAAGTCATTAAAAACCTACATAGCA
GGGTTCTTGTGAAGATCAAGTGATAATGTAGGAAGCATGTACAAATGTCA
CATTCTGCCGTCACGTAATGGTCCTCACAGCTTGAGGTAGCATTTAGCAT
GTGTCATGATTTAGTACAAGGGTTGGCAAACTGTTGCTCTTGGATTAAGT
CTGGCTCATTGCCTGTTTTTCAAAGAAAAAAATTGTATATGTGTGTATAT
ATGTTATATATAGGTACACACACATATGTGCTATATATAGCATATATACA
CACATAATATATAAACATGTACATATATAGCATTATATATATACCGTGTA
TAATATCTCCAGTCCTCATGACCAGCCATGCTTGTTCATTTACATTTGCA
TACTCTATGATTGCTTTCATGCAACAATGGCAGAGTTGAGTGATTGTTTT
GCACAGANACTGTATGGCCCACTAAACCTAAAATATTAATCTCTGCC
SEQ ID NO: 108
CTCCTGCCGGGCTCGTGGCGGCTTCTGTCCGCTCCGCGGAGGGAAGCGCC
TTCCCCACAGGACATCAATGCAAGCTTGAATAAGAAAAACAAATTCTTCC
TCCTAAGCCATGGCATATCAGTTATACAGAAATACTACTTTGGGAAACAG
TCTTCAGGAGAGCCTAGATGAGCTCATACAGTCTCAACAGATCACCCCCC
AACTTGCCCTTCAAGTTCTACTTCAGTTTGATAAGGCTATAAATGCAGCA
CTGGCTCAGAGGGTCAGGAACAGAGTCAATTTCAGGGGCTCTCTAAATAC
GTACAGATTCTGCGATAATGTGTGGACTTTTGTACTGAATGATGTTGAAT
TCAGAGAGGTGACAGAACTTATTAAAGTGGATAAAGTGAAAATTGTAGCC
TGTGATGGTAAAAATACTGGCTCCAATACTACAGAATGAATAGAAAAAAT
ATGACTTTTTTACACCATCTTCTGTTATTCATTGCTTTTGAAGAGAAGCA
TAGAAGAGACTTTTTATTTATT
nt: 682
SEQ ID NO: 109
TGCCACTGAAGATCCTGGTGTCGCCATGGGCCGCCGCCCCGCCCGTTGTT
ACCGGTATTGTAAGAACAAGCCGTACCCAAAGTCTCGCTTCTGCCGAGGT
GTCCCTGATGCCAAGATTCGCATTTTTGACCTGGGGCGGAAAAAGGCAAA
AGTGGATGAGTTTCCGCTTTGTGGCCACATGGTGTCAGATGAATATGAGC
AGCTGTCCTCTGAAGCCCTGGAGGCTGCCCGAATTTGTGCCAATAAGTAC
ATGGTAAAAAGTTGTGGCAAAGATGGCTTCCATATCCGGGTGCGGCTCCA
CCCCTTCCACGTCATCCGCATCAACAAGATGTTGTCCTGTGCTGGGGCTG
ACAGGCTCCAAACAGGCATGCGAGGTGCCTTTGGAAAGCCCCAGGGCACT
GTGGCCAGGGTTCACATTGGCCAAGTTATCATGTCCATCCGCACCAAGCT
GCAGAACAAGGAGCATGTGATTGAGGCCCTGCGCAGGGCCAAGTTCAAGT
TTCTGGCCGCAGAAGATCCACATCTCAAAGAAGTGGGGCTTCACCAAGTT
CAATGCTGATGAATTTGAAGACATGGTGGCTGAAAAGCGGCTCATCCCAN
ATGGCTGTGGGGTCAAGTACATCCCCAATCGTGGCCCTCTGGACAAGTGG
CGGCCCTGCACTCATGAAGGCTTTCAATGTGC
SEQ ID NO: 110
TCCCGGAATCGCGGCCGCGTCGACCTTGTCCTTGAGCGTCAACCTTCTTT
CCCTGAAGTGGCTGGGGTTCCTGTTTCCTTCTTTGATTGACAACTTGTGT
TAACCCTCGCACATCTCTGGGCCAATTTTTGCTTGTAAGTCTTTCCGGAG
ACCCCTGGAATTTAAATCATTAGCACCGCGCCCTTCCCCGAAGAGTCTTC
GAAGGGTTGCCGCTTTTCGGTGGCGCAGTTCTCGCGAGAAGGTGACTTTC
TTTCTCGGTATTTCCTGGTTTCCAGAATCCTTAGCGCGAGGCGGAAAAAA
TATTTCTCCCAGCTTGTGTTGATGCCGCGATTTTGACTGAGACTTCTTCC
CACGATTTCTGTTTTTGCTTCTCCAAGGAAAATGGCAGCTCCCGAGCAGC
CGCTTGCGATATCAAGGGGATGCACGAGCTCCTCCTCGCTTTCCCCGCCT
CGGGGCGACCGAACCCTTCTGGTCAGGCACCTGCCGGCTGAGCTTACTGC
TGAGGAGAAAGAGGACTTGCTGAAGTACTTCGGGGCTCAGTCTGTGCGGG
TCCTGTCAGATAAGGGGCGACTGAAACATACAGCTTTTGCCACATTCCCT
AATGAAAAAGCAGCTNTAAAGGCATTGACAAACTNCATCAACTGAAACTT
TTAGTCATACTTTAATCG
nt: 536
SEQ ID NO: 111
CAGAGATCAAAATAGGCCTTACACAGTGCGACGCGAATTTAAAAGATTAC
CCCATTCAGGTGTATGGATTTTGCAGTATTAAAGATGCTGCCTGGAATAG
GTCATTATCTTCTCCAAGTACTCTGTTAAGTCAATGAGTCACATAGAGTA
TAAGGTTTATTATCTGCTTTTCTTTCATTAAATAAATCTTTATTGAATTT
CTACTACATTAAAAAACCAAACCAAAACAAAACAAACAAAAAAAACACTT
CCCTGAGCCATAAAGGAGAAGGTAGTTTTGACTGGAACCTTGAAGGATGG
GTAAACTTTCAGCAGATAAAGATTGAGAGAAGACCTTCCAGGTAGAGAAA
GCAGTGTGGGCACAGGCAAAGATGGAAGAACACACGTGGCTGTGGGAAAC
ACAGCTAGAAGCCAGTGCGGATAGAGAGTAGGCTATGATGTGCAAAGGTT
ANACACTGGGAGAGACAGGTCCATGAGAGTAGCTTGGACTAACACAGGGA
GGGTTTGGAATCCCAACTGGGGAACCTANAAATCAA
SEQ ID NO: 112
TAGGAGGCTTATTCACTGATTTCCCCTATTCTCAGGCTACACCCTAGACC
AAACCTACGCCAAAATCCATTTCACTATCATATTCATCGGCGTAAATCTA
ACTTTCTTCCCACAACACTTTCTCGGCCTATCCGGAATGCCCCGACGTTA
CTCGGACTACCCCGATGCATACACCACATGAAACATCCTATCATCTGTAG
GCTCATTCATTTCTCTAACAGCAGTAATATTAATAATTTTCATGATTTGA
GAAGCCTTCGCTTCGAAGCGAAAAGTCCTAATAGTAGAAGAACCCTCCAT
AAACCTGGAGTGACTATATGGATGCCCCCCACCCTACCACACATTCGAAG
AACCCGTATACATAAAAT
SEQ ID NO: 113
TCTTTATCAAGTTGAGAAAGTTCCTCCCCTCTATTCCTAGTTTGCTAAGA
GTCCTTCTATCCTATTTCTTAATGGTTTAGTAGATGACTCTGTGGTACTT
TGAAGGTTGTTTGCAGAATTTCCATGCCATAGGCAATTTACCTTTCCTTG
ACATTTGAAGGATTGATGTTGGTGCCAAGTATAGAATCTTCACAGAGTCC
TCCTGTAGCTTCTAAAGGTTTAGCTTGAAAATGTTAATTGCTTAACGCTA
GTAAGTGAGTGAAAAAGCTGGGGATAAATTTTGTATCTTGCTTATATTTC
AGTTCCCACCTCTGTCCNGACNAAACCCCCATATATAA
SEQ ID NO: 114
TAGTTTACATATCCCAACCTTTAAAAATATTCCTCTTATTAGCTTTATAT
TCACTTTATAGAAGTTGAGTTTTAATTAAAATTCTTGGCATCCTGAAGTA
TGTCACATAGCATGTGCTCCTTATAAATATGTTGATATCTCAGAAGACAG
CATCCCGGTTTTCATTTTATAAAGTACCATACTTAAGAATGCTGTAATAC
TTATCTTTTATAACATGTTTCCTTCGCTTTGCTTGNCTTTTATGNCATCA
GTTTTAACTGTTTACTTCATTTAACAGNTTACATCATNCAACAGTTTACT
TCATTAAACAGTAGGTGGAAAAATAGATGCCAGTCTATGAAAATCTTCCC
ATCTATATCAAAATACTTTCAAGGATATACTTT
nt: 615
SEQ ID NO: 115
CGACTTTCAACCATCAAGTGAGGAATACCTTCACATAACTGAGCCTCCCT
CTTTATCTCCTGACACAAAATTAGAACCTTCAGAAGATGATGGTAAACCT
GAGTTATTAGAAGAAATGGAAGCTTCTCCCACAGAACTTATTGCTGTGGA
AGGAACTGAGATTCTCCAAGATTTCCAAAACAAAACCTATGGTCAAGTTT
CTGGAGAAGCAATCAAGATGTTTCCCACCATTAAAACACCTGAGGCTGGA
ACTGTTATTACAACTGCCGATGAAATTGAATTAGAAGGTGCTACACAGTG
GCCACACTCTACTTCTGCTTCTGCCACCTATGGGGTCGAGGCAGGTGTGG
TGCCTTGGCTAAGTCCACAGACTTCTGAGAGGCCCACGCTTTCTTCTTCT
CCAGAAATAAACCCTGAAACTCAAGCAGCTTTAATCAGAGGGCAGGATTC
CACGATAGCAGCATCAGAACAGCAAGTGGCAGCGAGAATTCTTGATTCCA
ATGATCAGGCAACAGTAAACCCTGTGGAATTTAATACTGAGGGTGCAACA
CCCCATTTTCCCTTCTGGAGACTTCTAATGAAACANATTTCCTGATTGGC
ATTAATGAANAGTCA
Sequence ID 469
GATTTTTAAAAATACATATAGCAAAAATATTACAGGGTCAGGGGAGACAA
TTAGAATGATATAATTCAAAGTGGATTAAAAAAAAAACTGTCACCCAGAA
TACAATACCCAGCAAAGTTGTCCTTCATAAATGAAAGAAAAATNAAATCT
TTNCCNAACNA
SEQ ID NO: 117
TCCCGGGAATCTGCAGGATCCGTCGACT
SEQ ID NO: 118
GACAGTGCCCAGGGCTCTGATATGTCTNTCACANCTTGNAAAGTGTGAGA
CAGCTGCCTTGTGTGGGACTGAAAGGCAAGATTTGTTCCTGCCCTTCCCT
TTGTGACTTGAAGAACCCTGACTTTGTTTCTGCAAAGGCACCTGCATGTG
TCTGTGTTCTTGTAGGCATAATGTGAGGAGGTGGGGANACCACCCCACCC
CCATGTCCACCATGACCCTCTTNCCACNCTNACCTGTGCTCCCTCCCCAA
TCATNTTT
nt: 694
SEQ ID NO: 119
TGGGCTTTGGGCTGGCTGCAGTCTGTCTGAGGGCGGCCGAAGTGGCTGGC
TCATTTAAGATGAGGCTTCTGCTGCTTCTCCTAGNGGCGGCGTCTGCGAT
GGTCCGGAGCGAGGCCTCGGCCAATCTGGGCGGCGTGCCCAGCAAGAGAT
TAAAGATGCAGTACGCCACGGGGCCGCTGCTCAAGTTCCAGATTTGTGTT
TCCTGAGGTTATAGGCGGGTGTTTGAGGAGTACATGCGGGTTATTAGCCA
GCGGTACCCAGACATCCGCATTGAAGGAGAGAATTACCTCCCTCAACCAA
TATATAGACACATAGCATCTTTCCTGTCAGTCTTCAAACTAGTATTAATA
GGCTTAATAATTGTTGGCAAGGATCCTTTTGCTTTCTTTGGCATGCAAGC
TCCTAGCATCTGGCAGTGGGGCCAAGAAAATAAGGTTTATGCATGTATGA
TGGTTTTCTTCTTGAGCAACATGATTGAGAACCAGTGTATGTCAACAGGT
GCATTTGAGATAACTTTAAATGATGTACCTGTGTGGTCTAAGCTGGAATC
TGGTCACCTTCCATCCATGCAACAACTTGTTCAAATTCTTGACAATGAAA
TGAAACTCAATGTGCATATGGGATTCAATCCCCACCATCGATCATAGCAC
CCCCTATCAGCACTGNAAACTCTTTTGCATTAAGGGATCATTGC
SEQ ID NO: 120
GGCAGCGCGGGGAGCCCGTCGGCGCCGGCGGGCGGGCCGGTTTCGAAGTT
GATGCAATCGGTTTAAACATGGCTGAACGCGTGTGTACACGGGACTGACG
CAACCCACGTGTAACTGTCAGCCGGGCCCTGAGTAATCGCTTAAAGATGT
TCCTACGGGCTTGTTGCTGTTGATGTTTTGTTTTGTTTTGTTTTTTGGTC
TTTTTTTGTATTATAAAAAATAATCTATTTCTATGAGAAAAGAGGCGTCT
GTATATTTTGGGAATCTTTTCCGTTTCAAGCATTAAGAACACTTTTAATA
AACTTTTTTTTGATAATGGTTAAAAAAAAAAAAAAAA
SEQ ID NO: 121
CATAATAAAAAACAATCAACAAACAGGGAATGGAAAGAAACTTCCTCAGC
ATGGTGAAGGCCACATATGAAAATCCCACAGCTAACATCATACTCAATGA
TGAAAGACTGAAAGCTTTTCTCCTGAGATCAGGAACAAGACAAAGATGTC
ACCTTTTGTCACTTCTATTCAACTCATTATTGGAAGTTTTTGCCAGAGCA
ATTAGGTAAG
nt: 476
SEQ ID NO: 122
CAGAATCTTTTCATAGGCTGAATGTTGCTCCACAATGTGTCCTTTGACTA
TCTCTGGCTAATTATTATTTTAATCTCTTCTCAGCTTTTCCAAGAACATA
ACGTTAACCAAAGATCTTAGGCCATTCACAACTCTTTTGTAAAAATTAAT
GTGGATGTGAAACGAGGCAACAAATCCTGAAGTAGAAAGTTATTCCTGGC
CAGGCACGGTGGCTCACGCCTGTAATCCTGGCACTTTGGGAGGCCGAGGT
GGGTGGATCATGAGGACAGGAGATCGAGACCATCCTGGCCAACATGATGA
AACCCCATCTCTACTAAAATACAAAAAATTAGCTGGGCATGGTGACGCGT
GCCTGTAGTCCCAGTTACTCGGGAGGCTGAGGCAGGGGAATTGCTTGAAC
CTCGGAGGTGGGAGGTTGCAGTGTGCCGAGATCACGCTACTGCACTCCAG
CCTGGCAACAGAGCAAGACTCCATCT
SEQ ID NO: 123
AAACAGAAAGTTTCTTCTAAAGGCATGATTCAGTTAAGTCATTCTTAAGT
GTTAAAAAATTGTGAAAAATGTGCCTGTAATCCCAACACTTTGGGAGGCC
GAGGCAGGCAGATCACGAGGTCAGGAGATCAAGACCATCCTGGCTAACAA
GGTGAAACCCCGTCTCTACGAAAAATACCAAAAACATTAGCCGGGCGTGG
TTGTGGGCGCCTGTAGTCCCAGCTACTTGAGAGGCTGAGGCAGGAGAATG
SEQ ID NO: 124
TTCTTGGGATATTGATGACTACTGTCTGAGAGGTGCTGTGGGGAGATTTT
CAGGATTGTGTGGTCTTTGAGGGGGGTGTTTTTTTAAGACAACATTGACC
ACTGTCCACTGTCCACATGATCATTGTAAAATTGCAATGCCGCATGCTAG
TTGGTTACATAAGACATAATTCCAGTGATTGAAGGTGGTTACACTGTATG
GTGGTGTGTTCAAGATGGCACTGGCATCTTTGAGCAGAGCCTGGCTATGC
AGCATCATTTGAGTTTTTTAAACACCCTANAGGTCTGGTTGTTGTTGCTG
TTGTCCTTTCCTGTGAAAGTCACAANANAAGTTACAGTCCAGGTGAACCT
GGAGTTTATAGGTTGGTTTTGTTTCTGNTATATATATATATATATATATT
TTTTTTTTTTTTTAACATTTACCTGTAGTGCTGTAGCTGTTGATACTATC
ACCTGCATGCTATTTCTAGTGAGTGCTAAATACAGTATGGTCCAATGACA
ATAACAGCCCATGGTACTGCCAG
SEQ ID NO: 125
CATCAGTCTGTTATCCATGCTGACTTTCCGAAGACTTGCAGCTACTGCAT
TGATATCTTTCCTGCCAATAAGCAAAGTGTTGAACACTTCACAAAATATT
TTACTGAGGCAGGCTTGAAAGAGCTTTCAGAATATGTTCGGAATCAGCAA
ACCATCGGAGCTCGTAAGGAGCTCCAGAAAGAACTTCAAGAACAGATGTC
CCGTGGTGATCCATTTAAGGATATAATTTTATATGTCAAGGAGGAGATGA
AAAAAAACAACATCCCAGAGCCAGTTGTCATCGGAATAGTCTGGTCAAGT
GTAATGAGCACTGTGGAATGGAACAAAAAAGAGGAGCTTGTAGCAGAGCA
AGCCATCAAGCACTTGAAGCAATACAGCCCTCTACTTGCTGCCTTTACTA
CTCAAGGTCAGTCTGAGCTGACTCTGTTACTGAAGATTAGGGAGTATTGC
TATGACAACATTCATTTCATGAAAGCCTTCCANAAAA
SEQ ID NO: 126
CACACTTTCATGATAAAAACAGAACCTAGGAATGAAAAGAAATTATAGCA
ACATAATAAAGACCATATATGAGAAGCCCACAGCTAACATACTGTATGGT
GAAAAACTGAAAGCTCTTCCTCTAAGATCAGGAACAAGGCAAGGATGCCC
ATTCTTGCCACTTCTATCGAACGTAGTACTGGAAGCCCTAGCCAGAACAA
CTAGGCAATAGAAAGAAATTAAAGGCATCCATNTCAGAAAGGAAGAANCA
AAATGCTGTCTGTTTAANATGACA
SEQ ID NO: 127
TTTCTATANAAAAAAATTTTTTAAAATAATTGTAAAGTTAGATTTAAAAT
TGTAAAATATAAAATCACAAAGGAATGTACCCAATAAAATGTAAATGCNC
CATAAAAAAAAAAAAAAAAAAAAAAAAAAA
SEQ ID NO: 128
CGNTAACGTGCAATCCGCCGCACGCCAGCAAACTGGACAAACTCCGGGAT
CTCATCGAAGCGATTGAGCACCAGTACCAGAGTAATACCGGACTGATGTA
ACGAGGCGAGTCGCTCATCCAGCTTGCTGACGTGAGGCAACATCCAGGCC
ATCGAACGGNTCATCAAGAATCAACAAGTCAGGCTCCGACATCAGCGCCT
GACACAGCAGGGTTTTTCGCGTCTCGCCAGTGGAAAGGTATTTAAAGCGT
CNGTCGAGGAGGGCGGTAATACCGAACTGCTGCGCCAGTTGCATGCAACG
CGGTGCATCCTTTACTTCATCCTGAATGATCTCAGCCGTAGTGCGTCCGG
TGCCATCTTCGCCAGGGCCGAGCATATCGGTGTTATTCCGCTGCCATTCG
TCGCTGACGAGTTTTTGCAATTGCTCGAAGGAGAGACGAGTGATGTGGGA
AAACTGGCTTTGCCGTTCACCTTTCAAAAGCGGGAAGTTCCCCCGCCAGC
GCGCGGGCCAGGGCCCGAT
SEQ ID NO: 129
TTTTTTTTTTTTATTCTATTAAAAAATGTTNNTGAAAAAAGATACTTAAA
TTTTAAAGATAACTNAATTCCTAANGATTTAAAATAATCCAAGCAGAGAT
GAAAGANCAAATGCAAATGCNTAAAAAGACCCCANAGCATTGTTAGCAAA
AAGCAAATATAGTTAGCCAAGCATATATATNTCATAAAAGCAATAANAAG
GCNTAAAGCAAGTTTGGGGAGAGCTTATTTAAAACTTGTAAAAATCATTT
GAATTTTTAAAAGTTTTCAAAC
nt: 551
SEQ ID NO: 130
TTTGGAACACAAAGTTCCCTTTTTAGAAGAATAGGTATTGAGCCCTTGAG
CGTGGGTAGAAAGATAGAGACAGAGTGATTTGCAAAATAATGGAGGATCA
TATTTATATATGAATTTTCACTTATTTGAACTTTCAGATATCANCTTNAA
AANCTTTGGTTTAAGTAAAGTNTNTTAATGAGACTCCTTGGATGAAAGTA
ACCAAAACCAGTAAAAATAAGGTAATAAGGATGTAATAGTTTCTTATGGA
CACTCAACAGCTAGAATGCAGTTAGTCTCAGAAAAGAATTAGAACAAATA
ACTGGAAGGCCATCAGGAGTCCAAAACCATCACTCTTTTATATTTTATAT
TTTATTTTTCTCTCTTCANATGAGCATTCTCTTTCTATGTCCATATGGTA
NAAGGCGGCAGCTCCATAGATTATGGCTTCAGATGTTACAGTTCCGCTNA
ATGCAGGGACAGACTTGCTATCTTTCAGTCCCCTTACATATCCTGGGGAG
AGAGCAAATGATTGACTGGCTTGAGTCAGGTGCCCGTTCCCTTTCCAAT
CT
nt: 224
SEQ ID NO: 131
GTTTGNTTGTGACCATCTGTACTTGTAATTTCTTTACNTTCATTGGTATG
AAAAATATGTTCTTAGAAGCANGAAAAAGAATTCAGNTTTGCTTTGTATA
CTAAATTAAATGCTGTAATTTTGATAAAATGAAAAATCTGCTTTATTTGC
AACAATTGGTTTCTTCCTTGACGTCAGCCTCACTCTTGGACTTTGGTATT
CAGCCNGNCACCCCTGGGAATTCC
nt: 349
SEQ ID NO: 132
GTGCCTCCCTGTGTGAGTAGCCTAAGGTGCATTGAAAAAGACTGGGATGT
GTTTTATTTTTTTGTATTAGATAGCATTAACCTTACTGTTGAAGTATTTT
TGGTGGAGTATTAGTGACAAGCCATTGAGTCTTAAGCCTTACGGCTTCCT
ATAAAATCACTAATTTCGTGTGTGTTTGTGTGTAGGTTACGTTATATATA
GGATTCGTGTTCGCCGTGGTGGCCGAAAACGCCCAGTTCCTAAGGGTGCA
ACTTACGGCAAGCCTGTCCATCATGGTGTTAACCAGCTAAAGTTTGCTCG
AAGCCTTCAGTCCGTTGCAGAGGANCGAGCTGGACNCCCTGGGGGGCTC
SEQ ID NO: 133
TTAACAGCTGCATAGAGTTTTAAAAGTACATTATATTTTGTCAGACAAGT
AAAATATCTGTTTTTCACGCAAAAAAAGCCATGAAATACGTAATTTTTTA
AAGACAAAAAATCATCTTTTGAGTTTGCTCTTTGGTTTTTCTTCATTCCT
TTTGAGGATTGGGAAAACAGAAAGATTCTTTGATTTGGGTAATGAAGAGG
TAATTTGGGACAGTGTGGTGGTACCAGGAAGAAAGAGGATTGGAAAGGCC
AGTACTGTTTTAGTTGCTCGGCACTGTTGGTTTTGTTTTAATGTGGTTGC
CCTGTCCACTACATGGTTCTATCAGTAGTGTAATCCATTTTCAATGTAAA
GCTCTTTTAGTTTTTGTCATAGACATAAATTAATATTTTGAGAGGCATCC
CTCACCTGTTCATTTCTTCTGTGTTGAAATGAAGTACTTAAAATTACCGT
TATACATGAACTTTGTGGACTGTAAGATTTGTTATATATGTTCAAATGCC
TTTTAGCTGGCTTTTTAATTAATATGCCTGTTTTGAGTGCTTAATACAAT
GTAATGNGGATTGTAAATCATACCTATTTTAAATCATTCCTTCCTGTATA
TTTGNACTCAGAGAGCCTTATTTTATTCTTCCAGC
nt: 382
SEQ ID NO: 134
TTTTCTTAGAACTTTATTTTTTCTGGCCAGGCGCAGTGGCTCACACCTGT
AATCCCAGCACTTTGGGAGGCCAAGGCAGGTCGATCACCTGAGGTCAGGA
GCTCAAGACCAGCCTGGCCAACATGGTGAAACCCTGTCTCTACTAAAAAT
ACAAAAATTAGCTGGGCGTGGTGGCGCATGCCTGTAATCCCANCTACTCA
GGAGGCTGAGGCAGGAGAATTGTTTGAACCCGGGAGGCGGAGGTTGCANT
GAGCCGAGATTGCGCCACTGCACTCCAGCCTGGGCAACAGAGCGAAACTC
CATCTCAAAAAAAAAAAAAAAAAACAACCTTTATTTTTTCTGATTTTAAA
AGTAATAACTAGTTTGTAGAAACATTAAAAGT
SEQ ID NO: 135
ACCCTAAACATAACTTAAAATTTGTTNGGAATTTGAAAGTACAGAATTTT
CCTGTAATTGAGACTNTTTAAACTTTTGTGGTTGGAGAAGGTATTCTATT
TTTTGAAAATATCTGTAAGTTTTATCTAAATAGTAAACTCTAAGTATTCT
TCCCCTTTACTTACAGCCACCCTGGGAATCTGAGACTAGAGAAAATAAAG
TTTGTCTCTTGTTCTAAGGAGGGTCTGGTTTAGAAATCTGATTTAGACAT
AGAAAAATTGCAAGAAGCTTGAGGTGATTGGAAGATACGATTTTGTTATC
AAAGNATGTTTCTGTTTTATAGATTTTATTCATCTACAACTCCTTATTAA
TATATTTAAGAAGTCATTAACCCACCATTGATTACTTGATATAAAAGGAG
AANCGGTGGTAAAAGGTGAAATANAATTTTTAATTTTTTTTTTTTTAAGT
TTAGGATTTTTTTTTAAATTCTAAGAGTTTCTGTCATTTGGGGACAATCA
GAA
SEQ ID NO: 136
TGGGAATCATAATTNGTTAACTGAAGCTNATAAGATGAGAGCATTCANAG
AGAAAAGAACGGAAAGATTGAATATCAGTTTCCCTTCTTTAAAAAAATTG
TGGATATGTGATCTAGCTTCTTGAGCATCACAGTGACTGATTGGCTCGTG
GTAATTGATCGCTATGCTGACAATCTTATCTCCACCTATGTCATTCAATT
TTCTAAGAGGCAAAATCCTTAATCAGGAGGAGAGTTTAGCTCTAGCTAAA
TTTCCCTTGTCCAGCATGCTCCTGCTCCCCCAACTTGTGGAAACAGCTAA
AGGATTGGACTAGGAGCANAAGTTTGGAATGGTTAAAATGTAGCAACATG
TGTTTCCTGAAACAAAATTCCACTATAATAAAAAAAGCATTTGAATGCTC
CCTTGTAATTCTGTTGGAGCTTGTTGCCTTTTTTATGACACAACCATAAT
CAGTGATAGACAGTAGCATAAAGAAGCAAGAGCAAAGCAATTAAGTAATA
ATAGCACTACAAAAATGTGTGCTGTACTTACCAAACACGACATTTATGAA
TTATTANATAGGAATAAGGGGATGGT
SEQ ID NO: 137
GACCCAGCCATCTAAATAAGTTRTACATGTTGCGTATTTTTTTGTTAGGG
ACTTATCTTCCGAAGAGGAAAGGTTTATGAAACCTAAAGTAACAATGATA
GCTTGGAATCAAAATGATAGCATTGTTGGCACAGCTGTGAATGATCATGT
CCTCAAAGTGTGGAATTCTTACACTGGACAACTGCTTCATAACTTAATGG
GACATGCTGATGAAGTATTTGTTCTGGAGACACATCCCTTTGATTCCAGA
ATTATGTTATCTGCAGGACATGATGGCAGCATATTTATATGGGATATTAC
AAAAGGTACCAAGATGAAACATTATTTTAATATGGTAAGTGAAGTGAGAT
GTACCTTGATACATGCTTGATAATTTGTTTAGAGTATTTGGGTTATGCGG
CTTACCCAGAAATTGATCTGCTTGTTTTGGCAGTTTGTTTTTACAAATCA
ACATATTCAAAGCCTGCTAAATATTAGACAGCTACATGTATATACGTACA
TACATGAA
SEQ ID NO: 138
TTTC
SEQ ID NO: 139
CTCGCTGGCGGGAGGCCACGGGCTTTCCACAGCGCGGGGGAACGGGAGGC
TGCAGGATGGTCAAGCTGACGGCGGAGCTGATCGAGCAGGCGGCGCAGTA
CACCAACGCGGTGCGCGACCGGGAGCTGGACCTCCGGGGGTGATCTGGAC
CCTCTGGCATCTCTCAAATCGCTGACTTACCTAAGTATCCTAAGAAATCC
GGTAACCAATAAGAAGCATTACAGATTGTATGTGATTTATAAAGTTCCGC
AAGTCATAGTACTGGATTTCCAGAAAGTGAAACTAAAATTTTAATCCAGG
TGCTGGTTTGCCAACTGACAAAAAGAAAGGTGGGCCATCTCCAGGGGATG
TAAAAGCAATCAAGAATGCCATAGCAAATGCTTNAACTCTGGCTGAAGTG
GANAGGCTGAANGGGTTGCTGCAGTCTGGTC
SEQ ID NO: 140
GAAGACCTCACATCTGAGAGCTCATCTGCGTTGGCATTCTGGAGAACGCC
CTTTTGTTTGTAACTGGATGTACTGTGGTAAAAGATTTACTCGAAGTGAT
GAATTACAGAGGCACAGAAGAACACATACAGGTGAGAAGAAATTTGTTTG
TCCAGAATGTTCAAAACGCTTTATGANAAGTGACCACCTTGCCAAACATA
TTAAAACACACCAGAATAAAAAAGGTATTCACTCTANCAGTACAGTGCTG
GCATCTGTGGAAGCTGCGCGAGATGATACTTTGATTACTGCAGGAGGAAC
AACGCTTATCCTTGCAAATATTCAACAAGGTTCTGTTTCAGGGATAGGAA
CTGTTAATACTTCCGCCACCAGCAATCAAGATATCCTTACCAACACTGAA
ATACCTTTACAGCTTGTCACAGTTTCTGGAAATGAGACAATGGGAGTAAA
TATTACACAAATACTTATTCATTGNGGTTATTTTTATACAGTAGTGAGAA
GAATATTGTTCCTAAGTTCTTAGATATCTTTTTTTGGATGTGCAAAAATT
TTTGGATTGACAGTAACTTGGGTATACATGACACTGAAATGCCTTACTTT
GGATGA
SEQ ID NO: 141
TGCCTGCGGGCCAGGACCTCGCCCAGCCCATGTTCATCCAGTCAGCCAAC
CAGCCCTCCGANGGGCAGGCCCCCCAGGTGACCGGCGACTGAGGGCCTGA
GCTGGCAAGGCCAAGGACACCCAACACAATTTTTGCCATACAGCCCCAGG
CAATGGGCACAGCCTTCCTCCCCANAGGACCCGGCCGACCTCAGCGCCTC
CTGCAGGCTAGGACACTGGTGCACTACACCCCATGCCTGGGGGCCGAGAT
TCTCCAGCAGAAAGATGCAATATTTTTTGTTTCCTTTTTTTCCATTTTTT
TCTCTAAGGAATCAATATTTCAATATGTTGAGTGTGTGTCCAATGCTATG
AAATTAAAATATTAAATAACATATTTATGGCATTTTCTTGAAGAGTGTGG
TTGAAGAAATATTTCTCCTTTTGTTTTTCTTTTTTTTTTGNTTGNTACTG
CCACTTCTTTTTAGGAGCAAATCTCCCCAGGGGTGTACGGNATTTCTTGA
CTCTGGGAACAGCTGCTACCCCCAAGACTTGCCACGTTGTTCTGCCCTCA
AATGGAATTAAGTG
nt: 390
SEQ ID NO: 142
GGAATATGGTCAGGATCTTCTCCATACTGTCTTCAAGAATGGCAAGGTGA
CAAAAAGCTATTCATTTGATGAAATAAGAAAAAATGCACAGCTGAATATT
GAACTGGAAGCAGCACATCATTAGGCTTTATGACTGGGTGTGTGTTGTGT
GTATGTAATACATAATGTTTATTGTACANATGTGTGGGGTTTGTGTTTTA
TGATACATTACAGCCAAATTATTTGTTGGTTNATGGACATACTGCCCTTT
CATTTTTTTCTTTTCCAGTGTTTAGGTGATCTCAAATTAAGAAATGCATT
TAACCATGTAAAANATGANTGCTAAAGTCAGCTTTTTAGGGCCCTTTGCC
AATAGGTANTCATTCAATCTGGTATTGATCTTTTCACAAA
SEQ ID NO: 143
ACCCGCCATCTTCCAGTAATTCGCCAAAATGACGAACACAAAGGGAAAGA
GGAGAGGCACCCGATATATGTTCTCTAGGCCTTTTANAAAACATGGAGTT
GTTCCTTTGGCCACATATATGCGAATCTATAAGAAAGGTGATATTGTAGA
CATCAAGGGAATGGGTACTGTTCAAAAAGGAATGCCCCACAAGTGTTACC
ATGGCAAAACTGGAAGAGTCTACAATGTTACCCAGCATGCTGTTGGCATT
GTTGTAAACAAACAAGTTAAGGGCAAGATTCTTGCCAAGAGAATTAATGT
GCGTATTGAGCACATTAAGCACTCTAAGAGCCGAGATAGCTTCCTGAAAC
GTGTGAAGGAAAATGATCAGAAAAAGAAAGAAGCCAAAGAGAAAGGTACC
TGGGTTCAACTAAAGCGCCAGCCTGCTCCACCCAGAGAAGCACACTTTGT
GAGAACCAATGGGAAGGAGCCTGAGCTGCTGGAACCTATTCCCTATGAAT
TCATGGCATAATAGGTGTTAAAAAAAAAAAATAAAGGACCTCTGGG
nt: 109
SEQ ID NO: 144
ACATTTTCCGGNCCTTTTGCCATACACAGTTACAGAGATCAGTCAAATCC
ATACCACCACTGAGATCTCATTTATTGCCACAGATGCACAAAATAAATAA
CCCAAAATC
nt: 374
SEQ ID NO: 145
CCAGCAACGACCCATACCTCAGACCCGACGGCCCGGAGCGGAGCGCGCCC
TGCCCTGGCGCAGCCAGAGCCGCCGGGTGCCCGCTGCAGTTTCTTGGGAC
ATAGGAGCGCAAAGAAGCTACAGCCTGGACTTACCACCACTAAACTGCGA
GAGAAGCTAAACGTGTTTATTTTCCCTTAAATTATTTTTGTAATGGTAGC
TTTTTCTACATCTTACTCCTGTTGATGCAGCTAAGGTACATTTGTAAAAA
GAAAAAAAACCAGACTTTTCANACAAACCCTTTGTATTGTANATAAGAGG
AAAAGACTGAGCATGCTCACTTTTTTATATTAATTTTTACAGTATTTGTA
AGAATAAAGCANCATTTGAAATCG
SEQ ID NO: 146
GTACAGGAGGTAAATTGGATACCCCATCTAAGGGGATCTGTGAGACCAGG
TAGTTATTTGGAATGAAAGAGTAAGATATTAAACCAGCCAGCATGTCAAC
AGGTGGGTGATAGTCTTGTTCTCACAGACAACAGATGGCCATCATCTTAA
AACAACATTTATGTTAACCAGCAGATAAGGGACTCCTGCATTGTCAGTGG
ACTTTGAGCCTGAGTTTTTCTACTTGCATAGGTGAAAGTGGACTGCAATG
CTAGTATAAATGCCGTATGATGACTAGTACCCCTTAGGGAGCTCCAGTTT
GCCTTCCTGGGGAACCACAGACCCCAAGTGTAATTTCCTGAGGACAGCCC
GACTTCT
SEQ ID NO: 147
GTTACTGTGAGCCTGTCAGTAGTGGGTACCAATCTTTTGTGACATATTGT
CATGCTGAGGTGNGACACCTGCTGCACTCATCTGATGTAAAACCATCCCA
NAGCTGGCGAGAGGATGGAGCTGGGTGGAAACTGCTTTGCACTATCGTTT
GCTTGGTGTTTGTTTTTAACGCACAACTTGCTTGTACAGTAAACTGTCTT
CTGTACTATTTAACTGTAAAATGGAATTTTGACTGATTTGTTACAATAAT
ATAACTCTGAGATGTGTGAAAAAAAAAAAAAAAAAAAAAAAAA
nt: 521
SEQ ID NO: 148
CTGCGGTGGAGCCGCCACCAAAATGCAGATTTTCGTGAAAACCCTTACGG
GGAAGACCATCACCCTCGAGGTTGAACCCTCGGATACGATAGAAAATGTA
AAGGCCAAGATCCAGGATAAGGAAGGAATTCCTCCTGATCAGCAGAGACT
GATCTTTGCTGGCAAGCAGCTGGAAGATGGACGTACTTTGTCTGACTACA
ATATTCAAAAGGAGTCTACTCTTCATCTTGTGTTGAGACTTCGTGGTGGT
GCTAAGAAAAGGAAGAAGAAGTCTTACACCACTCCCAAGAAGAATAAGCA
CAAGAGAAAGAAGGTTAAGCTGGCTGTCCTGAAATATTATAAGGTGGATG
AGAATGGCAAAATTAGTCGCCTTCGTCGAGAGTGCCCTTCTGATGAATGT
GGTGCTGGGGTGTTTATGGCAAGTCACTTTGACAGACATTATTGTGGCAA
ATGTTGTCTGACTTACTGTTTCAACAAACCAGAAGACAAGTAACTGTATG
AGTTAATAAAAGACATGAACT
SEQ ID NO: 149
AAGCTCATGATTTTAAATGTATTTTTCTAATAAACTATACTCCCATTTAA
AAATCACCAATACCTTAATGTTTCAATTATATAAGCTAATTAAAAATAAA
GGCTGGGCGTGGTGGCTCACTTTGGAAGACCGAGGCAGGCAGATCACCTG
AGGTCAGGAGTTCGAGACCAGCCTGCCCAACATGGAGAAACCCCATCTCT
ACTAAAAATACAAAATTAGCCAGGCATGGTGGCACATGCCCGTAATCCCA
GCTACTGGGGAAGCTGAGGCAGGAGAATCACTTGAACCTGGGAGGCAGGG
GCTGCAGTGAGCCGAGATCATGCCATTGCACTCCAGTCTGGGCAACAATA
GTGGAACTCCATCTCAAAAATAATAAAAAAAATAAAATAAAAATAAAATT
CAAACCTAAAATAGATGCTCTACTTCAGGAGTGGGCAAATTAATCACCTG
CATCCTTTTTTTGGGCTTTC
nt: 575
SEQ ID NO: 150
TTTTTTTCTAAATGGNGATTACTAATATATGTGGAGACTATTAATCTCTT
TTCTGTTGCCATTAGTTCATTTTTCCCCAAAAGCCAATACATGTTCATTA
CAAAAATGAATTATAAAATATAAGTTAAAAGAAAAACATAAAACCCTACA
ATCTTACCCACCCAGACAACTACTATTAATACCTTAGTATTAACATATAC
ACATCATGTATATGTATAAATTTATCTTAAACAAAAATAAAATTATTCTT
TACATATTGTTTTAAAACCTATTTATCTGGCCAGGTGCCGTGGCTCACGC
TTGTAATCCCAGCACTTTGGGAGGCTGAGGCACGTGGATCACCTGAGGTC
AGGAATTCGAGACCAGCCCAGCCAACATGGTGAAACCCTGTCTCTAATGG
TTTAAATACCAAAAAATTAGCTGGGCATGGTGGCACATGCCTGTAATATC
AGCTAACATGGGAGGCTGAGGCAGGAGAATCACTTGAACCANGGAGGGGG
AGGTTGCAGTGAGCCGAAATCACACCACTTCACTGCAGCCTGGGCAACAA
AGCAAGACTGTCTCAAAAAGAAAAA
SEQ ID NO: 151
CACTGTCATTCCCAGGAGGCTTTGGAGTCAGAACTGGATTCAAATTCTGA
CTNTATGTTGTGTGACTTGGGCCAATAGCTTCTTTNTGTGCCTCAGTTTC
TTTAGCTGTAAATANACGGGTAGGTCACCCCTTACCCCATAGGTTATGGG
GAAAGTTACAGAAAATGGTCAGCTGGGCNCAGTGGCTCAAGCCTGTGGTC
CCAGCNCCTTGGGAGGCCAAGGTGAGCAGATTGCTTGAGCCCAGGAGTTT
GACACCAGTNTGGCAACGTGACGAAACCCTATCNCTGTGAAAAATACAAA
AAATTAGCCAGGCATGGTGGTGTGTGTCTGTGGTTCCAGCTGCTTGAGAG
TTTGAAGTGGGAGGATCACCTGAGCCCAGAAGGTCGAGGCTGCAGTGAGC
TGTGATCGCGTCACTGCACTCCAGCCTGGC-GACAGAGTGAGA-CCCCT-
TTTGAAAAAAAAAAAAAAAAAAT
SEQ ID NO: 152
GTGAGCGGTGGTGGTTTATTCTTCCGTGGAGTTAAGGGCTCCGTGGACAT
CTCAGGTCTTCAGGGTCTTCCATCTGGAACTATATAAAGTTCAGAAAACA
TGTCTCGAAGATATGACTCCAGGACCACTATATTTTCTCCAGAAGGTCGC
TTATACCAAGTTGAATATGCCATGGAAGCTATTGGACATGCAGGCACCTG
TTTGGGAATTTTAGCAAATGATGGTGTTTTGCTTGCAGCAGAGAGACNCA
ACATCCACAAGCTTCTTGATGAAGTCTTTTTTTCTGAAAAAATTTATAAA
CTCAATGAGGACATGGCTTGCAGTGTGGCAGGCATAACTTCTGATGCTAA
TGTTCTGACTAATGAACTAAGGCTCATTGCTCAAAGGTATTTATTACAGT
ATCAGGAGCCAATACCTTGTGAGCAGTTGGTTACAGCGCTGTGTGATATC
AAACAAGCTTATACACAATTTGGAGGAAAACGTCCCTTTGGTGTTTCATT
GCTGTACATTGGCTGGGATAAGCACTATGGCTTTCAGCTCTATCAGAGTG
ACCCTAGTGGAAATTCGGGGGATGGGAAGGCCACATGCATTGGAAATAAT
ANCGCTGCAGCTGTGTCAATGTTGAAACAAG
SEQ ID NO: 153
TTTTTTTTTTATAAACTCCAATCATTTCCAGAGCTACTTAGCTCAGCATC
TTTTTTTTCCACGCTCTTAAGTTGTGTTTATACATTTTTGATACAGTTAG
ATTGTTTTTGTCACATTCTTCATTCTATCCTGGGATCCCCCAACCACCTA
AGTGGATTTTTTGATAATTTGCATGCTTTAAGGATAACTCTTCATTCTGN
AAAGGGCTATGGGTTTTGGCAAATGCAGAGTCATGTATCCAAGATTACAA
TATCGCACAGAAGAGTTTCATCACTATATAAAACTCACCAGTCTTCCTCC
TATTCAACCATCTCCATGCCTTCTTCCCAGCCCTAACTCCTTAAAACCAC
TCATATCTTTACTATTGCTATAGTATTGCCTCTTCCACCATGTCATATAA
ATGGAAACATACAGTATTAGTCTTCTCAAACTAGTTTCTTTTACCTAACA
ACATGCATTTAAGATTCATAGTGTCTTTTAATGACTTGATAGATTATTTC
TTTGTAGCTGAATAATATTGCATCTTATAGATGTAACCGTTTGTATATCC
ATATTTTCTCACAGCCTATGACTTGNCTTTTGATTCTCTGAACAGGCCAT
TCACAAAGCAGAAGTTTTAATTTTTATAAAGCTAATGNATCAACTT
SEQ ID NO: 154
CCTGGATGACAGCATATCTGTTTATAGCTCAGTTTACTGAATACTTTAAG
CCCACTGTTGAAACCTGCT
nt: 502
SEQ ID NO: 155
GATGCATGTCCAGCATAGGCAGGATTGCTCGGTGGTGAGAAGGTTAGGTC
CGGCTCAGACTGAATAAGAAGAGATAAAATTTGCCTTAAAACTTACCTGG
CAGTGGCTTTGCTGCACGGTCTGAAACCACCTGTTCCCACCCTCTTGACC
GAAATTTCCTTGTGACACAGAGAAGGGCAAAGGTCTGAGCCCAGAGTTGA
CGGAGGGAGTATTTCAGGGTTCACTTCAGGGGCTCCCAAAGCGACAAGAT
CGTTAGGGAGAGAGGCCCAGGGTGGGGACTGGGAATTTAAGGAGAGCTGG
GAACGGATCCCTTAGGTTCAGGAAGCTTCTGTGCAAGCTGCGAGGATGGC
TTGGGCCGAAGGGTTGCTCTGCCCGCCGCGCTAGCTGTGAGCTGAGCAAA
GCCCTGGGCTCACAGCACCCCAAAAGCCTGTGGCTTCAGTCCTGCGTCTG
CACCACACATTCAAAAGGATCGTTTTGTTTTGTTTTTAAAGAAAGGTGAN
AT
SEQ ID NO: 156
CTGCGATNGAGTTTTGAGAGGAAGGANTAAAGTNCTCATCTCNGACGGTG
AGAAAGATCATNACTAAGGAAACGCAGGGTTGGAAGCAGTGCTGANTGTC
CAGTTGAGTTTCATGANCAAACATTTGCTGTGGGACCAGTTTTCATGGNG
GTTTGTCATTTTGTCCAGCTGCCTGGAGCTGCTTGGTTGAAGGCACAGAA
TAATCAGGATTAATTGTTNAACTTGTATGAATTTCTTTATTTTAAAATAG
GAATAATATCTGCCTTGGGAGCAAGTTGTAAGAGTTAACTGAAAGCTTNA
GGAAAAACTTTCCCTTGCTATTTAAGTAGGGCTTTACAAGTTACAATTCT
ATCACAGTTTTAAGATTATAAAC
SEQ ID NO: 157
GCGGCGCANCTGCGGATCCANAAGGNCATAAACGANCNGAACCTGCCCAA
NNCGTGTGATATCACCTTCTNAGATCCAGACNACCTCCTCAACTTCAAGC
TGGTCATCTGTCCTGATGAGGGCTTCNACAAGAGTGGGAAGTTTGTCTCA
AAAAA
nt: 585
SEQ ID NO: 158
GATTTACTGTGGGAATTTGCTCATGCAATTATGGAAACCTAGAAGTCCCA
TAATATGCCATCTTCAAGCTGGAATCCCAGGAAAGCAGGTGGTGTAATTC
TGAGATTGAAGTCTTGAGAACCGGGGGAGTCAATGGTGTAACTCCCAATC
TAGGGCTTAAGGCCCAAGGACCAGGGCTGCTGGTGTGCAGATGCAAATCC
TGGAGTTCAAAGGATTGAGAACCAGGAGCTCTGGTGTCTGAGGGCAGTAG
AAGATGGATGTTCCAGCTCAAGAAGGGAAAGTAAGAATCCGTCCTTCCTC
CACTTTTTTGTTCTATTCAGATGAGCCCTCAATGGACTGAACGATGCTCA
CCCACACTGTGAGGGCTGGTCTTCTTTATTCAATCCACTGACTTAAGTGC
TGATCTCTTCTGGAAACACCTTCACAGACACACCCAGAAATAATGTTCTA
CCAGCCATGGGCCTGTTACTTAGCCCAGTCAAGTTGACACAGAAAATTAG
CTATCACAACATCTGTGTGTGTATATACATATGTATTTGCATGTGTGTGT
ATATATGGNGTATATATATTCATGTGTGTGTATAT
SEQ ID NO: 159
CTTTTGCCAGTAGGCCCCCTGAGTAGGTTCCTCTATCTTTTGGCATGACC
CCAGAAGTCTTTGATAACTTCCTTGCTTTCTGATGTGACAAGACATCCAG
GGCCAGATTGTCCATATCCTGCCCCGGATGCACGATGCACTGTTTCTCCA
AGAATCCCTGTGTCCTTTGCTGATGATGCCATGATTTTAAGTTCTCTAAT
ATAGTTTTATCTCTTTGTTTCAGATAATGCTTTTGTGTTCTCACATGTCC
TGCTCTCTCTCTCTCTCTCATTTTGGTGTTGATCAGTCTTTCCATAAGAT
TGTTTATTTCACTAGTCCTTCATTCTTCTTTTTTCTAAATTTACTCTTCT
TGACTAGTATCCTGTCACTTCTGAGGACTCATATTTTTGCAACTTGAAAA
TTATTCTTATTTATTTAAGTATATGTTNCTGAAACTCTCATTAGACACAT
TTTG
SEQ ID NO: 160
GTTAAAAAAAGTAAAAGGAACTCGGCAAATCTTACCCCGCCTGTTTACCA
AAAACATCACCTGGTAGCATCACCAGTATTAGAGGCACCGCCTGCCCAGT
GACACATGTTTAACGGCCGCGGTACCCTAACCGTGCAAAGGTAGCATAAT
CACTTGTTCCTTAAATAGGGACCTGTATGAATGGCTCCACNAGGGTTCAN
CTGTCTCTTACTTTTAACCAGTGAAATTGACCTGCCCGTGAAGAGGCGGG
CATAACACAGCTGAAAAAAAAAAAAAAAAAAAAAAAAAAAAAAAAAAAAA
AAAAAAAAAAAAAAAAAAAAAAAAAAAAAAAAAAAAAAAAAATTTT
nt: 516
SEQ ID NO: 161
CTTTTCATGGTCTCTTGTTCATTAATCATCTAAAATCCAAGCNCAGAGAA
TTCAATTTTAGATGGTCTCCAGAGCAGAATTTGATGTATAATCTTAATTA
CAAATCATAGATAATTAATATTGNTTACAAAATCANAATACGATTAGAGG
TAGGGATCCTGCACACACCCTATTTTCCTCCCCAGTGTTCTGACCGAGAG
ACTAATTAATAATTCAAGGAACTTACAGTGAATGANAACCCATGGTTTTG
CTTAATTATCAGAACAGCTAGATCTGAGAACAGCTGTCTCCCACATGGAT
AGACACTTATTCCACCCATTTGCAGGTAGAATAGCTGGCAATAATAAGTC
CTTCCCATTGGATATGTTGAAAGGTGCCTGCCATGGCATAGTTGCCACAA
GAGAGGAAGAAATGGACACAAATGTAGGCTGTTTTCAGGGCANAGGGAAG
GTGGGAGGAAACCAANTTGCTGGTTTTCACACACCCTCTGGGGAACACCC
ATGCACCTATGANATG
SEQ ID NO: 162
GACAAAAGCTGAGAGAATTTTTTTCTTGAATATTTGCACTAAAAGATAGG
TTAAAATTCTTCAGGCTGAAGAGAGCATACCAGGTGGAGATTTGGATCTA
CAAAAAGGAAGGAAGATTTGGAAATGGATTTGGCACCATTGACTCAATTT
CCAGAACAAGAAAGCAGGGACAGTTTTGGGAAGCTCAAGACACACTGCCC
ATGAGCAGCAATTTGGACCTCCTGCTGCATCCACTGTGCATCAAACACAC
ACTGTACAGACAAAGACTCCCAGGAAAAGAAGTATAAACATGGACTAACA
CAGAGATGGGCAAACTACAGCCTGTGACCCAGCCACCTGTTTATGTAGAA
TCCAAAGTAAGAATCTTTAACTTACACATAAACTT
660nt
SEQ ID NO: 163
GACAGCAGAGCACACAAGCTTNTAGGACAAGAGCCAGGAAGAAACCACCG
GAAGGAACCATCTCACTGTGTGTAAACATGACTTCCAAGCTGGCCGTGGC
TCTCTTGGCAGCCTTCCTGATTTCTGCAGCTCTGTGTGAAGGTGCAGTTT
TGCCAAGGAGTGCTAAAGAACTTAGATGTCAGTGCATAAAGACATACTCC
AAACCTTTCCACCCCAAATTTATCAAAGAACTGAGAGTGATTGAGAGTGG
ACCACACTGCGCCAACACAGAAATTATTGTAAAGCTTTCTGATGGAAGAN
AGCTCTGTCTGGACCCCAAGGAAAACTGGGTGCANAGGGTTGTGGANAAG
TTTTTGAAGAGGGCTGAGAATTCATAAAAAAATTCATTCTCTGTGGTATC
CAAGAATCAGTGAAGATGCCAGTGAAACTTCAAGCAAATCTACTTCAACA
CTTCATGTATTGTGTGGGTCTGTTGTAGGGTTGCCAGATGCAATACAAGA
TTCCTGGTTAAATTTGAATTTCAGTAAACAATGAATAGTTTTTCATTGTA
CCATGAAATATCCAGAACATACTTATATGTAAAGTATTATTTATTTGAAT
CTACAAAAAACAACAAATAATTTTTAGATATAAGGATTTTCCTGGATATT
GCACGGGAGA;
SEQ ID NO: 164
GACAGCAGAGCACACAAGCTTNTAGGACAAGAGCCAGGAAGAAACCACCG
GAAGGAACCATCTCACTGTGTGTAAACATGACTTCCAAGCTGGCCGTGGC
TCTCTTGGCAGCCTTCCTGATTTCTGCAGCTCTGTGTGAAGGTGCAGTTT
TGCCAAGGAGTGCTAAAGAACTTAGATGTCAGTGCATAAAGACATACTCC
AAACCTTTCCACCCCAAATTTATCAAAGAACTGAGAGTGATTGAGAGTGG
ACCACACTGCGCCAACACAGAAATTATTGTAAAGCTTTCTGATGGAAGAN
AGCTCTGTCTGGACCCCAAGGAAAACTGGGTGCANAGGGTTGTGGANAAG
TTTTTGAAGAGGGCTGAGAATTCATAAAAAAATTCATTCTCTGTGGTATC
CAAGAATCAGTGAAGATGCCAGTGAAACTTCAAGCAAATCTACTTCAACA
CTTCATGTATTGTGTGGGTCTGTTGTAGGGTTGCCAGATGCAATACAAGA
TTCCTGGTTAAATTTGAATTTCAGTAAACAATGAATAGTTTTTCATTGT
nt: 660
SEQ ID NO: 165
GACAGCAGAGCACACAAGCTTNTAGGACAAGAGCCAGGAAGAAACCACCG
GAAGGAACCATCTCACTGTGTGTAAACATGACTTCCAAGCTGGCCGTGGC
TCTCTTGGCAGCCTTCCTGATTTCTGCAGCTCTGTGTGAAGGTGCAGTTT
TGCCAAGGAGTGCTAAAGAACTTAGATGTCAGTGCATAAAGACATACTCC
AAACCTTTCCACCCCAAATTTATCAAAGAACTGAGAGTGATTGAGAGTGG
ACCACACTGCGCCAACACAGAAATTATTGTAAAGCTTTCTGATGGAAGAN
AGCTCTGTCTGGACCCCAAGGAAAACTGGGTGCANAGGGTTGTGGANAAG
TTTTTGAAGAGGGCTGAGAATTCATAAAAAAATTCATTCTCTGTGGTATC
CAAGAATCAGTGAAGATGCCAGTGAAACTTCAAGCAAATCTACTTCAACA
CTTCATGTATTGTGTGGGTCTGTTGTAGGGTTGCCAGATGCAATACAAGA
TTCCTGGTTAAATTTGAATTTCAGTAAACAATGAATAGTTTTTCATTGTA
CCATGAAATATCCAGAACATACTTATATGTAAAGTATTATTTATTTGAAT
CTACAAAAAACAACAAATAATTTTTAGATATAAGGATTTTCCTGGATATT
GCACGGGAGA
SEQ ID NO: 166
GAATTGTGATAGTTCAGCTTGAATGTCTCTTAGAGGGTGGGCTTTTGTTG
ATGAGGGAGGGGAAACTTTTTTTTTTTCTATAGACTTTTTTCANATAACA
TCTTCTGAGTCATAACCAGCCTGGCAGTATGATGGCCTANATGCAGAGAA
AACAGCTCCTTGGTGAATTGATAAGTAAAGGCAGAAAAGATTATATGTCA
TACCTCCATTGGGGAATAAGCATAACCCTGAGATTCTTACTACTGATGAG
AACATTATCTGCATATGCCAAAAAATTTTAAGCAAATGAAAGCTACCAAT
TTAAAGTTACGGAATCTACCATTTTAAAGTTAATTGCTTGTCAAGCTATA
ACCACAAAAATAATGAATTGATGAGAAATACAATGAAGAGGCAATGTCCA
TCTCAAAATACTGCTTTTACAAAAGCAGAATAAAAGCGAAAAGAAATGAA
AATGTTACACTACATTAATCCTGGAATAAAAGAAGCCGAAATAAATGAGA
GATGAGTTGGGATCAAGTGGGATTGANGANGCTGTGCTGTGT
SEQ ID NO: 167
CTTGAACCTCGGAGGCAGAGGTTGCAGTGAGCCGAGATCACGCCACTGCA
CTCCAGCCTCGGGGACAGAGCAAGACTCCATCTCAAAACACACACACACA
CACACACACACACACACACACACACAAAACAGATATACACTGAACACAGC
ACAAGTGGGACATAAGAGATTTAAAAGGGTTAGAGATGTAAAATGGATCT
AGGAATGGAAACCATAAGGNGGGATTTATCAACTGGATTCTGCANAATGC
TGTTAAGGCCAGATGTTAGCAGGTGTTACATAAAAAAGGGATACCATGAG
CAAAAGTATTTGAACATGGGCAATGGTTGAAACAAGTTTAAACAGATTAT
NTTTATTACCAAATCTCTCAAACCTTTAATATGCTATAAACATTGTGAAA
CAATAAAAAAACTTTCCAAAA
SEQ ID NO: 168
GGGAAGGGAGCTATGAGTGTGTGTGTTGTGTATGGACTCACTCCCAGGTT
CACCTGGCCACAGGTGCACCCTTCCCACACCCTTTACATTCCCCAGAGCC
AAGGGAGTTTAAGTTTGCAGTTACAGGCCAGTTCTCCAGCTCTCCATCTT
ANAGAGACAGGTCACCTTGCAGGCCTGCTTGCAGGAAATGAATCCAGCAG
CCAACTCGAATCCCCCTAGGGCTCAGGCACTGAGGGCCTGGGGACAGTGG
AGCATATGGGTGGGAGACAGATGGAGGGTACCCTATTTACAACTGAGTCA
GCCAAGCCACTGATGGGAATATACAGATTTAGGTGCTAAACCGTTTATTT
TCCACGGATGAGTCACAATCTGAAGAATCAAACTTCCATCCTGAAAATCT
ATATGTTTCAAAACCACTTGCCATCCTGTTAGATTGCCAGTTCCTGGGAC
CAGGCCTCANACTGTGAAAGTA
SEQ ID NO: 169
GGCGGAGGTTGCAGTGAGCTGAGATGGCGCCATTGCTCTCCCAGCCTGGG
TGACAAGAGCAAAACTCCGTCTCAAAAAAAAAAAAAAAAAAAAAAGCAAT
TTACTTAAAAACATACAAACACAGAGACAAGTATTTTTGAGAAACAAATA
CCTTTTTCATTTTTTATACCAATGTAACAATAATCCATTAAACACACCTT
TACTAACTGTTTTCTAGGAGTCTGATATGATGAGGAAATAGGTAAACCTT
TAATAGCCAGTACTAAATTAGAGTGGCACAACTTTCACTGGGAAAAAAGA
TGGGTATTTTACTTTTCTGTTTTAGAAAAGTGGCTTGACAACAGTATGCT
TATGTCTTAGAGTTTGAAATTCAAGTTCTTGAACATTATTAATGGCTACA
ATCATTCATACCCACATTGGGCTGTATTCTTGATGAATCCAAAGTGATTT
TCACCTCAACTCTGAATTTCATTCTCCTCTTTTGAATATAATACAACCAT
CTCACTAGAGGAAGCATTTCAGTCTTTTCTGATTGGAGATTCATTATTGT
TTTAGATAATGTTTTCATTTGCTTATGGGTATATAAAAAATTTTATCTTA
AAAATATTTCCTCTCATTTAGCTAGCAACATTGTTTTC
SEQ ID NO: 170
CTCAGGGTGATCTCTGAACCCAAACTTGCCCCAAAGAAGGTTGCTCTGTC
CTCTCCACATCCCCATCTCCTCCCTAGGGCCTTGTTGGGGAGAGGCTCCT
CCATCTTTCCCAAGTCACACCATCGTTTCCTACGTGGTCTGGACAAGAGC
AAGAGCACACCTTGTCCCCACCTTCTCCAGAGCAGCCAGAACCCACCTCA
GGTGCCTTCCCCATCCGGTGCAGTTAAGGCACTTCTGCCAGCACCATGGT
ATGAGCACTAGACTTGGAGTTAAGATTTGAGAGCCCCCTCTGTCACTGTG
GAAGCTTGAGCATGTTGCTTGATCTCTCTGAACCTTGTGTTTCTCATCTG
TGAAAGGTGATAATGTGGGGCTGCTGTGAGATTTAAAGGACATAATGCAC
CTACGGTCCAAGCACTGCCTGGAATACAGCANAAGCTCAACAGATACTGG
ACAACCCATCCCCTTAGTAGAGGCACTAACCATGTGACCCAAGGCAAAAG
TGCTTAAAAAAA
nt: 580
SEQ ID NO: 171
ATTGCATGCAAGTTTGCTGAGCTGAAGGAAAAGATTGATCGCCGTTCTGG
TAAAAAGCTGGAAGATGGCCCTAAATTCTTGAAGTCTGGTGATGCTGCCA
TTGTTGATATGGTTCCTGGCAAGCCCATGTGTGTTGAGAGCTTCTCAGAC
TATCCACCTTTGGGTCGCTTTGCTGTTCGTGATATGAGACAGACAGTTGC
GGTGGGTGTCATCAAAGCACTGGACAAGAAGGCTGCTGGAGCTGGCAAGG
TCACCAAGTCTGCCCAGAAAGCTCAGAAGGCTAAATGAATATTATCCCTA
ATACCTGCCACCCCACTCTTAATCAGTGGTGGAAGAACGGTCTCAGAACT
GTTTGTTTCAATTGGCCATTTAAGTTTAGTAGTAAAAGACTGGTTAATGA
TAACAATGCATCGTAAAACCTTCAGAAGGAAAGGAGAATGTTTTGTGGAC
CACTTTGGTTTTCTTTTTTGCGTGTGGCAGTTTTAAGTTATTAGTTTTTA
AAATCAGTACTTTTTAATGGAAACAACTTGACCAAAAATTTGTCACAGAA
TTTTGAGACCCATTAAAAAAGTTAAATGAG
SEQ ID NO: 172
GCAACCTGCACAACCCCGCCCTGTTCGAGGGCCGGAGCCCTGCCGTGTGG
GAGCTGGCCGAGGAGTATCTGGACATCGTGCGGGAGCACCCCTGCCCCCT
GTCCTACGTCCGGGCCCACCTCTTCAAGCTGTGGCACCACACGCTGCAGG
TGCACCAGGAGCTGCGAGAGGAGCTGGCCAAGGTGAANACCCTGGAGGGC
ATCGCTGCTGTGAGCCAGGAGCTGAAGCTGCGGTGTCAGGAGGAGATATC
CAGGCAGGAGGGAGCGAAGCCCACCGGCGACTTGCCCTTCCACTGGATCT
GCCAGCCCTACATCCGGCCGGGGCCCAGGGAGGGGAGCAAGGAGAAGGCA
GGTGCGCGCAGCAAGCGGGCCCTGGAGGAAGAGGAGGGTGGCACGGAGGT
CCTGTCCAAGAACAAGCAAAAGAAGCAGCTGAGGAACCCCCACAAGACCT
TCGACCCCTCTCTGAACCAAAATATGCAAAGTGTGACCAGTGTGGAAACC
CAAAGGGCAACAGATGTGTGTTCAGCCTGTGCCGCGGNTTG
nt: 671
SEQ ID NO: 173
GGAATAGAATTTTAAATAGTAATAACTGCTTGTTTTTTTTGTGCAAGTAC
TTTTATACATAAGATAAACAAAAACCTTACCACCAAACATACCAAAATGC
ACCTCTTTCATAAGTGAGTTACTAAGATTTCTATACCTGGAATATCATGT
ATGTTTCATTTACTGGATGTTTACATTTTAGGAAGGAAAATAGTTTTGTT
TATTTAAACAACTGAATACTTATAAACTGTTGTTCCTGGAAGTTATTTAT
TCCATAAAAAATTTGTTCTTTTGTCATGAATTTATAATTCCTAAATGAAG
ACCAGAAAGTACAAATTGCTGGGAGGAAGAATAGGCTTTATTAATCAACT
GATGTCTTGATTTTTCTAAATGGGAAGATTGCTTTATTTTTAACACTAAT
TATGGGAGCAGATTCTTAGCAAACTTCTTTGGAAAAGTTAATGTTATGAT
GTGCATTAGGCTGCCCCATCGTGTATATAAATGAAGCAGATTTGATTTTT
GTATTCTTACGTTTCTCTGCTTTGTAGTTGTGGCTGTACTTAAAGAAATA
CAGAATTTCATATATTTAAAAATGTTTAAAATGTGACCCACAGACATTGT
AAATGGATTNAAAACTAACATGAAAAATATTCAACCTAAAAGAATTCTTA
ACTTCACAAGTGTTTTACTTC
SEQ ID NO: 174
CTTGGTTCCGCGTTCCCTGCACAAAATGCCCGGCGAAGCCACAGAAACCG
TCCCTGCTACAGAGCAGGAGTTGCCGCAGCCCCAGGCTGAGACAGGGTCT
GGAACAGAATCTGACAGTGATGAATCAGTACCAGAGCTTGAAGAACAGGA
TTCCACCCAGGCAACCACACAACAAGCCCAGCTGGCGGCAGCAGCTGAAA
TCGATGAAGAACCAGTCAGTAAAGCAAAACAGAGTCGGAGTGAAAAGAAG
GCACGGAAGGCTATGTCCAAACTGGGTCTTCGGCAGGTTACAGGAGTTAC
TAGAGTCACTATCCGGAAATCTAAGAATATCCTCTTTGTCATCACAAAAC
CAGATGTCTACAAGAGCCCTGCTTCAGATACTTACATAGTTTTTGGGGAA
GCCAAGATCGAAGATTTATCCCAGCAAGCACAACTAGCAGCTGCTGAGAA
ATTCAAAGTTCAAGGTGAAGCTGTCTCAAACATTCAAGAAAACACACAGA
CTCCAACTGTACAAGAGGAGAGTGAAGAGGAAGAGGTCGATGAAACAGGT
GTAGAAGTTAAGGACATAGAATTTGGTCATTGTCACAAAGCAAATGTGTC
GAGAGCA
SEQ ID NO: 175
GTCACCAAGAGCTTGTTGTCAGGTTTTCACTTGCTATTCGCAGAGATTTT
TTTTAAAGGCACTATTTGTAGTGTTAAAAGGGTGAATTTATCANAAGGCA
TAATAATCATAAATGTGTATATGCCTAATAATAGAACTTTAAAAGGCATG
AAGCAACACTCAAAAGGATTAAAGGGAGATCATCTCACCCCCTTCTTACC
AATTGATAGAATGATCTGATGAAAACAGTAAAATAACAACAGATCTGAAC
ACTGTCAACCATCTTGACAAATACTTATGCCTAGTGTTCCATTATTGGAA
CACTAAACATGTGGAATGATTTATATCCTACTGCTCAAGGTCATCACCAA
GGTCTAATTGTAAAATTTCAAAAAATTGCAACCTCAGGCATAAATGGGTT
AATCGACATTTATAGCACACACATGCAACATGTACCAGAGATTCCTTCTT
TTCTATGAACATGGTACTTCCACCAAGATAGACCACATTGTGAACTATAA
AACAAATCTAAAAACATTTGAAATGAAGGAAATTATATAAAATATGTTCT
CTTGATCTCAATGAAATTAAATTAATACTATAT
SEQ ID NO: 176
CTCATGGCGGCCAATGTAGGCCCAAAACTTCCTCAAGTCAAACTCTCCAG
GCCCACCTTCTGCTTCCCGGTGGCATCAACAGGCCCAGCTTTGACTTGAG
AACAGCCTCTGCAGGCCCTGCTCTTGCCTCCCAGGGGCTTTTTCCAGGCC
CAGCTCTTGCCTCATGGCAGCTGCCCCAGGCCAAATTTCTGCCTGCCTGC
CAGCAGCCTCAACAGGCACAGCTCCTCCCTCACAGTGGCCCATTTAGGCC
CAACTCATGACTGTGAGGCCATTTCCAGGCCTAGTGCCTGCCTCGTGGCT
GACTCTTGAAGCCCAAAACTTCCTCAAATCAGCCTTTTGCCCAACTTCTG
TCTACTGTCGGACTCTACAGGTCAGCCTCTGCCTCACAGTGGACCCTCCA
GACCCAGATGGTGTCTNCTGTGGCATCCTCAGGCGAAGCTCCTGCCTTTC
GGCAGCCTCTCCAGGCCCAGCTCCTCCTGCTCCAGCCTTCTCTCCAGGCT
CTGAACTTTCTCAGGTCTCCCTCTGTTGTCCAAGGCTGGAGTGTAGTAG
SEQ ID NO: 177
TATATATGTAATGCCCTTAACCTAGTGTTTGGCATGATCGTTGCTGAAAG
GGAAGCTTGTGGGTACAGTGTCCCCTCAGAAGCCAAAGCCCAGGGAAGGT
CGCCTGCCCAGGTCAGGCTCCCAGCGAGTTTGTCTGGGGAGGGGCCATTC
ATACCTCCAGGTCAGGACAGAGGCTCGGGCTGAGGGAACCCTACACAGGT
CCTGGAAGCAGATCCTTCCTGCCTAAGCCAGCAGGACAGCTCAACAGGAA
GCATCTTCCAGCCACGGGAGGAGAGGCAGCACCTTTTTTGGAACCATACA
GAGCTAAGAATGGTGGTACAAGTAATAGATTCTGTACTGGCAACCCCACT
TGGTGGAGCAAGTTCTAGGAAAAGGGGGCTGTCCTTGAGTCAGCCATGGG
GTCAGCCACACAGTCACCGCAGCTGCTCTTTGGCACCGGGCGCTGGAAAG
ACCTAGGATGACACAGCCTGGAAAGAGCTTGGGAAAAGCTCATCTTCCAC
AGAACTACCTGCTATACCAGCCAGGGCAGGTGCTTATTCCCACAACAGCC
CTCTGTTGTAGGCGGCAGTGCCATCCTGAANGTGCCGTGGTACCTTCTGA
ANACCCAGCTGAGGGCCTGTAATGGCACTTGCATGCCACATGGNACACCC
TTTCCCGGTTAA
SEQ ID NO: 178
ACCGCGGCCGCGTNAANAAAAAAAAAAAAAGAATTCCACTTGATCAACTT
AATTCCTTNTCTTTATCTTCCCTCCCTCACTTCCCTTTTCTCCCACCCTC
TTTTCCAAGCTGTTTCGCTTTGCAATATATTACTGGTAATGAGTTGCAGG
ATAATGCAGTCATAACTTGTTTTCTCCTAAGTATTTGAGTTCAAAACTCC
TGTATCTAAAGAAATACGGTTGGGGTCATTAATAAAGAAAATCTTTCTAT
CTTAAAAAAAAAAAAAAAAAAAAAAAAAAAAAAAAAAAAAAAAAAAAAAA
AAAAAAAAAAAAAAAAAAAAAAAAAAAAAAAAAAAAAAA
nt: 457
SEQ ID NO: 179
TTAGAGAGGTGAGGATCTGGTATTTCCTGGACTAAATTCCCCTTGGGGAA
GACGAAGGGATGCTGCAGTTCCAAAAGAGAAGGACTCTTCCAGAGTCATC
TACCTGAGTCCCAAAGCTCCCTGTCCTGAAAGCCACAGACAATATGGTCC
CAAATGACTGACTGCACCTTCTGTGCCTCAGCCGTTCTTGACATCAAGAA
TCTTCTGTTCCACATCCACACAGCCAATACAATTAGTCAAACCACTGTTA
TTAACAGATGTAGCAACATGAGAAACGCTTATGTTACAGGTTACATGAGA
GCAATCATGTAAGTCTATATGACTTCAGAAATGTTAAAATAGACTAACCT
CTAACAACAAATTAAAAGTGATTGTTTCAAGGTGATGCAATTATTGATGA
CCTATTTTATTTTTCTATAATGATCATATATTACCTTTGTAATAAAACAT
TTTTCCC
SEQ ID NO: 180
CGTCTATTTGNGTTTCTTCTCACAATTGGTAAGTTCTCTGTATTGATTGA
TGGCTAAGTTTGATTAGTGTTTTTCTCTAGTTGGTAATTATATTCTAGTA
TTTTATCATCTTATTGTTTACTCAACTNAAAGTGNCACAGAAGAGTTGCC
AGGTTTCTCTTTGATATGAGATCTCTNNTTGATTTGGAATGCAAATCANA
AGTGTCATGTTTTGAATAAAGGGACCAGATGACTTATAGGTATTCTTTCT
CTAAATATAACTAAGGTAAGATTTTTGTTTTGAGGTACTTAATCTATATA
AGTGGTAAAGAATTTACTTGAATTTCTCCAAATTCTCATGTCTAAAGTCT
GATTGATTAAATTCATTCTTGGTATTTCATTTTGAAAAGAATGTAGCTTT
AGCAAACCTCTTTGTATAAATGCAGTGGGATTAAGGTCATTTAAAAAATT
GTTATATCATTGTATTTTTAAAATTTACCAGTTTTATTTTTCTTTTTACC
CTTTAGCCCGGCCTCAGAAAGTGTGTTTGTGTCCATTTCTCCCAGCGCAC
CCTCTGCATATCTCTACCCACTTGTCATAATTCAGCATCCAGCAGAGGAA
AACAAAGTGTTGCGTACAGTTCCTCTACTAGCAGCATGCCTCCCCCAGGA
CAAGTGTA
SEQ ID NO: 181
TTATTGCTGACATAAAAATGGTGCACATCGGCCAGGGCCCAGGATGAATC
AGCCAATCTGCACCATTTATACATGGAACTGGAGAACATTGTGCCAATAA
TCATTTAATATATGCCAAATCTTACACGTCTACTCTAAACTGCTCTAATG
AAGTTTCAGTGACCTTGAGGGCTAAAGATTGTTCTTCTGGGTAAGAGCTC
TTGGGCTGGTTTTTCANAGCAGAGTTCTTGTTGTGGGTAGACTGTGACTA
GGTTCACAGCCTTTGTGGAACATTCCGTATAACGGCATTGTGGAAGCAAT
AACTAGTTCCTATGAAAGAACCAGAGCTGGGAAGATGGCTGGGAAGCCAG
GCCAAAGTGGGGGCAACAGCTTGCTTCTCTTTCTCTTCTCACCCTCAGTT
TGTATGGGAAAATGGAGATGTCCTCTCCACTTTATCCCACGATATCTAAA
TG
nt: 209
SEQ ID NO: 182
CAGGATATCGAGACCATCCCAGACAGCATGGTGAAACTCCGTCTCTACTG
GAATACAAAAAGTTAGCCGTGTGTGGTGGCACGCGCCTCTAATCCCAGCT
ATTCGGGAGGCTTAGGCAGGAGAATTACTTGAACCCGGGAGGCGAAGGTT
GCAGTGAGCTGAGATCGCACCATTGCACTCCACCCTGG-CGACAGAGCAA
GACTCCGTCT
nt: 541
SEQ ID NO: 183
CAGCCAACCCAGAAGGAGCCAGTCTACAACTATGCCTGATCCTCCTCATG
GCAGGCCACGAAGCATTGCTGCCATGTGTTGAATTATAAAACCCACATTG
CTTTTTGAACCCTGTTGCGGGTAAAAATAACCAAATTATCAGTCCTTGGA
AACCCAGGCAATCAAGTGAGTACAAGGTAAAGATAAGTATGGTTTAGAGG
AGAAATTATGTTCCTGAACTGGTGTCCTTTGATGGCAGCGTCAGCCTTGC
TAAGTCAGAGTAGAGGGAGCAGTGACCTTAATAAGCTTTGGTGAGCATCA
TGTGCACGCGTGGGTGGGAGTCCCTTTCACTGATGCTTTTAAAAGTGCTT
TTGCAGACCCTGGAAGGGATCCTCCACACATATGAGGTGTGGGACAGGTA
GGCCAGAGAGGATTAGCCCTGCTTTCGAGACTAGAAATCTACAGTCCTGA
AGGAGCAGTAATTAATTGGTACACCTGTCAGGGCCAGCCCCCAGGTCTCC
TGGCTTTTTCCAGGTTTTCTGTCTCACATGATTTTGCTTTT
SEQ ID NO: 184
CTTTAATTTTTCAAGTGTTTAAAAAACAATTTTATACTTAAGCCAGCCTT
GAAGATAAGCACAAAATTTACCAGTTTACATTTAAAAAACAAACAAAAAA
CGACAACAACTCAAGCACCCGCTCTGTGCATAGCACTATTCTAGGTGCAA
TAAAAGGGAATCTTAACCTTAGAAATATGAGTTCACTTTCTGGAATTGTA
TTATCTCCTTTTCCAGAGAGTAAAAATAAATAAAATCACCATTGTTTACT
ACAGATCTGCCCCAAACCACATCTGGTTCACAGAAAGGCTAATTTCTGCC
AAATTAAAGATGTAATGAACTCAGTTCCTGCTTTCCCAAAAACACGAAAG
CAGAATTCCTTTTCACTGAAAAAAATAAACAGTTTTCCATGCAAGGGCAG
TTTGCTTCTAATAAGTATTTTTTAAAAAATTTTTTTTTCCTCTAGCTTTT
CTTTAAATTTTCTTCCTCTAATATTGCCTTTTCTTGTACAAGGCAGACCA
GGTATCTTTTTATGCTGTTTTTCCTTTACTAAGAAAAGTATTGCATCTTG
AAGACAAACCATTTCCCAGAGTAGTGATAAAAAATAACACTAAAAAAACT
TTAAAGGTGAGTCACTTCATCACCTTGATGAAGTAAAAAA
SEQ ID NO: 185
GGAAAAAATATTTCCACTTAGATATTTTACATGGTTTTGTTTAAAATTAC
CATTACTTGTTTTTTAAAAACACATGACCACATATGTATATGTATATCTA
CCTAAACATTGTATCATGGTTTCAGTATGTTATTCATGTATTACTGGGAG
ATGCTACCAAGAAACCAACCCAAAGAAAATTCTGGAAAATACATTTCTAT
TTATAGAATAAATGTTTCATTTATATAAAAGCAAAAGAACTTAGAGTTCT
AATAAATGGGATGTCTAATAAATTATGAAGTTACTGATTTGAATATATTA
TATTTTTATAACTTCCTTGCCAAAGTCCTGATTTAGTACATTAGAGAACC
TGTGTTTCCTCTCTCCTCTACCATTCATCTCTCTTCCATACAGTCATTTG
GGCTTTTTACTCAAAGAGAATCAAGAAATAATAAGGTATAACAAGCTTGG
CAAAGTGTTGGCTTTTTAAAAAAAAATTTTTTTAATCTCTAGCAGTTTGG
TAATTTAGCAGCATCATTTATTTGGGATTCTTTTATCTGATTTCAACAGT
GAAAAACATCCCTATGATAAAGCCTAATGACCCATTTCCAAAAGATGGAA
TTGCCCTTCCTAGAAAATATGACGGAGAAAAGT
nt: 502
SEQ ID NO: 186
CGAATAGCCAAGTGGTCTGACAAGATCGAGAGTAATGAGGCCCATACTTT
AGTACAGTCTTGAATGGCCAGATGGTGCTGGGCATACCCCAACCAGAGAT
ATGTAAGTCTTTATGTTGTCAAAATTTCCCAGAAACATGAATTTCCCACT
AAGATTCATTAAGGAAAACTAGAATGAAAACAAAAACGTTCCTTGTATAA
TATTCATTANAAAGAAATGAAGAAGGCCGGGCATGGTGGCTCACGCCTGT
AATCCCAGCACTTTGAGAGGCCAAGGTAGGCAGATCATGAGGTCAGGAGT
TTGAGACCAGCCTGGCCAACATAGTGAAATCCCGTCTCTACCAAAAATAC
AAAAAAATTAGCCGGGCATGGTGGCACACACCTGTCATCCCAGCTACTCA
GGAGGCTGAGGCAGGAGAATTGCTTGAACCTGGGAGGTGGAGGTTGCAGT
GAGCTGAGATTGCACCACTGTACTACAGCCTAGGTGACAGTGCAAGACTC
TG
nt: 316
SEQ ID NO: 187
CCTATGCCAAACTAAAGAAAGCTTGCCTGGCCTACAGGCCTAAAGGTTCA
AATGNGGATTAAAAAAACACAGTAGTCACATAAAATGTCTGCTGGCTGGC
TGGAATTCCATCACCTACAATTTACCTGCTTTCAAAAACTGTGTTCAACA
TTGAGAAAACAGAAAACCACTTATCTTGAGCTTAATATGGGCTTCTTTTT
CCTTAACTGTAGAACACTTACTGAAATATCAAATCAATGGTTAGGATATG
TATCCTAGGCAGGCCTAAACCATTAACACTTGGTTTAAGCAACTTTGTAT
AATTNACCTCCTAAAT
SEQ ID NO: 188
CTTCATGAGTGCCCGGTTGCCCAAGTCAAAAACCTGGGAGTGATATAAAC
TCCCCACACATCCAGTCAGTCACTCATCAACTCTATTGATTCTG-CTGCT
AAATATATCTCAATTGTATTAACTTAAACATATGCATAATACATCTTCTT
CTTCACTGCATTTTTGTGGGCTGCACTTACCTTTCAGGTAACAACAACAC
TGGCCCCTCTTGCCCTTCTAGTCAGAAGTGCCAAAATGATGAGAGCTAGC
CATGACAAACCCACAGCCAACATTACACTGAATGTGCAAAACTGGAAGGG
CATCCAAACAGAGGAGG
SEQ ID NO: 189
TAGAATTCTCGCCTGCCTTGGCTTCTCCCTCTAGTTGTTCCTTCTCTGTC
TTCTGTGGGCTTCTTATTGTCTGCTCACTCCTTCTTCAGTGTCCTCTCAT
GGGCTTCCTTCCCTTCTCAGCTGATGCCATCACCTGGGGAATCACAGTTA
CTCAGCAGCACTGGGGCCTCTCTATCTCTATGCTGGTCATGCCTATGTGT
GAGCTGCAGACCCAGTGGAATTTCCATTTGTGCATCCCATGCCCAGCCCA
CCCTCCACCAGCCTCGAATGCAGCTGTTCAGCCCTACCCCAGTCCTCAGA
AAAGTTCCTCTCCCTGGATCCTCTTTTTCCTTCATGAGTGCCCGGTTGCC
CAAGTCAAAAACCTGGGAGTGATATAAACTCCCCACACATCCAGTCAGTC
ACTCATCAACTCTATTGATTCTGTCTGCTAAATATATCTCAATTGTATTA
ACTTAAACATATGCATAATACATCTTCTTCTTCACTGCATTTTTGTGGGC
TGCACTTACCTTTCAGGTAACAACAACACTGGCCCCTCTTGCCCTTCTAG
TCAGAAGTGCCAAAATGATGAGAGCTAGCCATGACAAACCCACAGCCAAC
ATTACACTGAATGTGCAAAACTGGAAGGGCATCCAAACAGAGGA
nt: 631
SEQ ID NO: 190
CTGAGGTGGGAGGATTCCACTCTCACCCATTTCTTCTTTCATTTTCAGTT
TCTCCAGTTAGTAACTGAAGATGTTCTTTGAGTAATTAAGTGAGTGAGAA
AATTTTTAAGTGAGAAATCTATAAAAAGAACCATGTTAACATAAATATTT
CAGTCCTTACAAGTTGGTATTGACTTTTCTCATTGGTAATCTGACTGATT
TAATACTGCTCATTCCAATATCTGGTGATGTAATTCTGGTTATGAATCCT
TGTATTAATAACACCTCCTGGGAGGTTTTTTTTCCCCAACATTACATTCA
GAATATTAGAGCTGAAAATACCTTTTTTAAGGTTATCAGGAGGAGGGAGC
TTATGTTTAATGTGGTGGATAAAACTTAACTGCTGGTTAATACAATTGTT
ATTCAGGTGAAATTCCCTAAACTTTTCACGTGCAAAGTTTTGTATGTATA
CAGACATTTGGGGAAAAGTTTTATCATCCCTAAAACCGGTTACTGTCCAG
AAAATGATAAGAATCCCTGGGTTCCAAATCCTTCATAAGGTATTTATTCA
TTTATTTATTCAACACATTTACTCAATGCCTCCGCTCTGCTGCAACTACA
CTGACATTCTGCTTCTAATCTAACCGAAAAT
SEQ ID NO: 191
TTTCAAATTGTACAATAACACAAACAACTTTGTTAAGGCCATGTTTTATT
TGCTGATTAATGGACAAAAGGCAATGTAATTTATTTTCAAGTATTTTCTT
GAAAGTCTGTGCTCATAAAAATCATGAAAAGTTGGAAAGACTGTTAAATC
ACTGAAACTTCAAATATATCTTACACAATCTTGTTTGTACAAAAATACAA
GTTAAATATAAACATAAAGCAATCATGGTAATTTTATGCAAATCTGTTTT
ATGTGATCATCAGTTATATATAAAAGTTTCTCAGTTCTGTTATTTGTGAA
AAGATCAATACCAGATTGAATGACTACCTATTGGCAAAGGGCCCTAAAAA
GCTTACTTTAGCACTCATCTTTTACATGGTTAAATGCATTTCCTAATTTG
AGATCACCTAAACACTGGAAAAGAAAAAAAATGAAAGGGCAGTATGTCCA
TAAACCAACAAATAATTTGGCTGTAATGTATCATAAAACACAAACCCCAC
ACATCTGTACAATAAACATTATGTATTACATACACACAACACACACCCAG
TCATAAAGCCTAATGATGTGCTGCTTCCAGTTCAATATTCAGCTGTGCAT
TTTTTCTTATTTCATCAAATGAATAGCTTTTTGTCACC
SEQ ID NO: 192
GTAAACTGTTCTCTCCGAGGGAAAAAATGGAAGTTATCCTCACAGTTCAC
TGCCGTGGTATTTCTTCTGTCCCATGCTTTGCATGACTGCCATGGTACAG
CCTTGTTTCAAACTGTTCACTGTGATCTGTGGGTCTTTGAGTTTCAGTGA
GTTTGCTGAAATGTCGAAGAAGTAGTTCCAAACTTCAATGTTCAATGAAA
TTTTTGTTCAAGTTTGAAATGGAGAGAGCAGCTTTAAAAGGTACTAAGCC
TTTTACAAATTGGTGAGTACTGGCACATGAGAT
SEQ ID NO: 193
TTTTTTTTTTTCCTTAAAAGGTAACCCCTAAACACAGCTAAAACTATGCC
ATCAGCTGACTCCAAGGNACACACAGTCCTGTATCTGGAACTACTGAGTG
GCAGGCATCTTTCTCTGCCTCTGACAGTGGAGTCCCCATCACTGCAGAGC
ATAGCCAAAGGAGTCAAAGGTCTCAGCGGGTCACTGCCTTATCAACCCTC
ACCAGTCCCTTATGTTTTTTAATATTTTATAATCTTGACATGACACCAAG
ATGCTTTAATAAAAAAGCACCTCTAACTCGGTCTTGTATTCACTTACCTT
GAGCCTGGGACTTCTCTAGGCTCCTGAGGCAAAAACAGGTAGAGGGGAGA
TGGTGGAACATAAAACACAATTTTGCTTGGCACCCACCTTGGCGTCTGTC
CCCATGACCAGGTCTTTCAATTCGATGATTTTGTCATTGATGGAGGAGCG
ATATCGTTTCTCAATGATATTATGGGTTGTCCGCCTTTCTCCTTCTTTGG
GGGGCTCAAGCTGCTTGACTCCCCCAGGTACCTGCTTAATGGGGCACTTT
CTCTTGCCCCATCATTACAGGCATTGTGGTCAGAATGGTCCCACTGCTGC
CCACCAGGGTCTA
SEQ ID NO: 194
CTAGTCTTTTCATAGTCTGCATAGAGTCTGGCCATTACCATCAGTTTTTA
AGATGTCCATATTGTGGCCGGGCGCGGTGGCTCACGCCTGGTAGTCCCAG
CACTTTGGGAGGCTGAGGCAGGTGGATCATGAGGTCAGGAGATCGAGACC
ATCCTGGCTAACACGGTGAAACCCGTCTCTACTAAAAAAAATATTAAAAA
ATTGGCCAGGCCTGGTGGTGGGCGCCTGTGGTCCCGGCTGCTTGGGAGGC
TGAGGCAGGANAATGGTGTGAACCCGGAAGTCGGAGGTTGCAGTGAGCCA
AGATTGCACCTGGGCAACACAGCGAGACTCCGTCTCAAAAAAAAAAAAAA
SEQ ID NO: 195
CAATTATTTATTACCTTTCCATTTGTTCGCCTGATGATGTGACAATGCAT
GGTCTTTGTGCATGCTGCTAGACACTTTTCTTTCCCAGCCGAAAAGTCTA
TTATGTAATTTTTACATTCATAATTTTAATGTGGATGATCAGGATTAAAT
CAAGATATATATCTGGAACCTCTTATAAATGGAGCACTTAGAAATTTGTT
GTTCTGCACTTAACCTAGAGAGAGAAAAAATGCTTTTCTTTGTGAAAAAT
CTGAATTCCTGTCCTGACCTTCTGTGATGTGGAAACCCTAGGCTCTGAGA
CACACTCTCTGGTGTCTGAGACAGAACCAAAGCAATAACGTTGTGATGCC
CACAGGCCTGGAGCCAGCTAGCGACCTTGTGCCGCCCAGCTGTCCATGGC
CCGTGCAGAGCAGAGGACAGTGAGTGTCTGCACTGAGAACCTTAAACCAC
AGTTGAACATACCCACACCTGTTTGTCTTAAGCTATAGTGTAAAAACAAA
GTTTGGGCTCTGAAAATTTAACTGAAAAAGATTTCCTTGTT
SEQ ID NO: 196
GTGGCAGCAGGCGCAGCCCAGCCTCGAAATGCAGAACGACGCCGGCGAGT
TCGTGGACCTGTACGTGCCGCGGAAATGCTCCGCTAGCAATCGCATCATC
GGTGCCAAGGACCACGCATCCATCCAGATGAACGTGGCCGAGGTTGACAA
GGTCACAGGCAGGTTTAATGGCCAGTTTAAAACTTATGCTATCTGCGGGG
CCATTCGTAGGATGGGTGAGTCAGATGATTCCATTCTCCGATTGGCCAAG
GCCGATGGCATCGTCTCAAAGAACTTTTGACTGGAGAGAATCACAGATGT
GGAATATTTGTCATAAATAAATAATGAAAACCTAAA
SEQ ID NO: 197
CAGCAGCAGAAATGTTTGCAAGATAGGCCAAAATGAGTACAAAAGGTCTG
TCTTCCATCAGACCCAGTGATGCTGCGACTCACACGCTTCAATTCAAGAC
CTGACCGCTAGTAGGGAGGTTTATTCANATCGCTGGCAGCCTCGGCTGAG
CAGATGCACAGAGGGGATCACTGTGCAGTGGGACCACCCTCACTGGCCTT
CTGCAGCAGGGTTCTGGGATGTTTTCAGTGGTCAAAATACTCTGTTTAGA
GCAAGGGCTCAGAAAACAGAAATACTGTCATGGAGGTGCTGAACACAGGG
AAGGTCTGGTACATATTGGAAATTATGAGCAGAACAAATACTCAACTAAA
TGCACAAAGTATAAAGTGTAGCCATGT
SEQ ID NO: 198
TACTCAATGAAAAACCATGATAATTCTTTGTATATAAAATAAACATTTGA
AAAAAAAAAAAAA
nt: 565
SEQ ID NO: 199
CAGGATCAAGGTGAAAAGGAGAACCCCATGCGGGAACTTCGCATCCGCAA
ACTCTGTCTCAACATCTGTGTTGGGGAGAGTGGAGACAGACTGACGCGAG
CAGCCAAGGTGTTGGAGCAGCTCACAGGGCAGACCCCTGTGTTTTCCAAA
GCTAGATACACTGTCAGATCCTTTGGCATCCGGAGAAATGAAAAGATTGC
TGTCCACTGCACAGTTCGAGGGGCCAAGGCAGAAGAAATCTTGGAGAAGG
GTCTAAAGGTGCGGGAGTATGAGTTAAGAAAAAACAACTTCTCAGATACT
GGAAACTTTGGTTTTGGGATCCAGGAACACATCGATCTGGGTATCAAATA
TGACCCAAGCATTGGTATCTACGGCCTGGACTTCTATGTGGTGCTGGGTA
GGCCAGGTTTCAGCATCGCAGACAAGAAGCGCAGGACAGGCTGCATTGGG
GCCAAACACAGAATCAGCAAAGAGGAGGCCATGCGCTGGTTCCAGCAGAA
GTATGATGGGATCATCCTTCCTGGCAAATAAATTCCCGTTTCTATCCAAA
AGAGCAATAAAAAGT
SEQ ID NO: 200
CAGAAGAGTAAGCAAATCTCAAAGCAGCGAAAGGGAAGAAACTAAAAAAG
GTAGAGCAGAAATAAGAGAAAATAGAGAAGAGAACAATTGAGAAAAATAA
TTGAAACCAAAAGGTGGTTCTTTGAAAAGCCTAACAAAATGGACACATCT
TTAGTTAGAGTGACCAAGAAAAAAGGGCAGTGACTCAGATTACTTCATTC
AAGAGTGAAAGAGGGCACATCACTACCAATTTACAGAAATAAAAAGGATT
ATGAGGAAATACTACAGATAATTGATGACATTAACTTAGAAGAATATATT
TCAAGAAAGACACAAACTACTGAAACCGACTCAAGAAGAAACAGAAAATC
TGAACAGACCTATAAAAAATAGAGATTTAATTGATATTCAGAAAGTTTCC
CAAAAAGAAAAGCACTGGCCAAGATGACTTCACTGGTGAATTCTATCAAG
TGTCAAAGATGAATTACTGACATTCATTCACACTCCTTTAAGAAATAGAA
GAGGGGACATCACTTTTCAAAGCATCGACATTCTAATCATTAGTCCCTTG
GTTTCCTGCTCCCAAAGCCAGGTGATGTATCACAAAAAAACCCCTACAGA
CCCACTGGGCACAATGGCTTTATGCCTAT
nt: 98
SEQ ID NO: 201
CTTTGCTCGAATNGTCAGATAAGGATTCTGTGAANGGAGATGAGATTTCC
ATCCATGCTGACTTTGANAATACATGTTCCCGAATTGGGGNCCCCAAA
SEQ ID NO: 202
CTCAAGTGTTCCCTCAGCTTAGGCTTTGTTTAAATGATCCCACCCAGGGG
CGATGGTAGGGAACAACAGGGTCACTAAACTATTTGGCTGGCTACAACTC
TGGGAAATGGTAAGACAGGGAAAGGCCATGTTGTTCATTCCCTTGTGCAG
ATCTAGGGAGAACCGCAGAGAGAACAGTTAGCATTTCTTGTTCAATGAAT
TATCCTATTAAGAACACTGGATGT
SEQ ID NO: 203
CGGNCGCGGTCGACGCTACTCCTACCTATCTCCCCTTTTATACTAATAAT
CTTATAAAAAAAAAAAAAANAAAAAAAAAAA
nt: 362
SEQ ID NO: 204
GGCATGTGCCTGTAGTCCTAGTTGCTGAGGTAAGAGGATTGCTTGAGCCC
AAGAGTTCAAGGCTGCAACAAGCTTTGATTGCGCCACTGCACTCCANCCT
TGGCGACAGACTAAAACGCTGTCTCAAAAAAAAAACAAAAACGACNAAAA
AAAAACAAAACAGAAAAAATTAACTTAGGCAATGACAGTCCCTGGCAAAT
GCTGGGAGGGAGGCAACANTGGTCAAGGAAGGTAACCCTGAANCAGGACT
TGTAAAGCAAATAANATTGGGAGGCCAAGGTGGGTGGATCACNAGGTCAG
GAGTTCGAGACCAACCTGGCCAACATAGTGAAACCCCGTCTTTCTAAAAA
TACAAAAAAATT
SEQ ID NO: 205
GACAAAAGAACCATTTGGATACATAGGTATGGTCTGAGCTATGATATCAA
TTGGCTTCCTAGGGTTTATCGTGTGAGCACACCATATATTTACAGTAGGA
ATAGACGTAGACACACGAGCATATTTCACCTCCGCTACCATAATCATCGC
TATCCCCACCGGCGTCAAAGTATTTAGCTGACTCGCCACACTCCACGGAA
GCAATATGAAATGATCTGCTGCAGTGCTCTGAGCCCTAGGATTCATCTTT
CTTTTCACCGTAGGTGGCCTGACTGGCATTGTATTAGCAAACTCATCACT
AGACATCGTACTACACGACACGTACTACGTTGTAGCTCACTTCCACTATG
TCCTATCAATAGGAGCTGTATTTGCCATCATAGGAGGCTTCATTCACTGA
TTTCCCCTATTCTCAGGCTACACCCTAGACCAAACCTACGCCAAAATCCA
TTTCACTATCATATTCATCGGCGTAAATCTAACTTTCTTCCCACAACACT
TTCTCGGCCTGTCCGGAATGCCCCGACGTTACTCGGACTACCCCGATGCA
TACACCACATGAAACATCCTATCATCTGGAG
nt: 595
SEQ ID NO: 206
TTCAAATTCTTGNTAANAGTCTTTGTTCTGAATTTTACTTTGTCTGTTAT
TCCTATAGCCTTTCCAATTTTCTTTCGCTTGGATTTTACGTGATAAGTTT
TTTCCCCCATTTTACTTTTANCAACTCTATATTTTTTAGTTGAGGTTGGG
TTTCTTGTAAACAGCATATAATTTGGGTTTTTTAATCCAATCTGAAAATT
AATGTCCTTAATTTTGTGTTTATACCATTTACACATAATGTACTCATATA
TAAGGTTTAACTGAAACCTACTATCTTGCTAGTTGTGCTCTACTTGAATT
TTTTTTTAGTATTCTGTTTTAATTGACCAACATTTGACTGTATCTCTTTG
TGTAATTCTTTTACAGGTTGCTGTAGGCATGACAATATATACACTTAACT
TTTCTCAGTACACTGAGAGTTGAAATTGTAGTACTTCGAGGAAAACATAG
AAAACTTGCAATGATATCGGTTACATTTTACCACCTCCATATGTTGCAAT
TATTAAATGTATTAGATCTGCCTACCTCGAAAACCCATCAGTCTTTTAAC
TTTGCTCTCAATGGTGATTCATATTTTTAAAAAAACTTGAGGCAA
nt: 522
SEQ ID NO: 207
TCGACCGGGTTTGGAGCAGTGCCTTGTTTGCTGTGCAGCGGATACTCTAC
AGGTACATTTCCTTTTTGGAACCAAAAGGGAGGGATTTGACAATATTGAT
GGTAGATCTTTTTTCTTTAGCAAGAATTAAGGATTTTGGTGGGTGGGGGG
AGGCTTCTGTGGGGACCAAGACAATGTACTGTCAGTCAGGATTTAAGTCG
AACTACCTCATCCCTTGCCCCAGAGAACAGTTGATCGTGTTTTAAACCAA
AAGGTGCGGAATGGAGAGAGGGAGGCGGTGCATTGCAGCTTCCGATAGAG
CTTTTTATTTTTGGATATCAGGAACCAATTTTGAAGATTTCTTAAGAAAG
TCATTTACATCAGGGACATGAAGAGCAAAGTAGGTATTTTTGGTCAGTAC
TTGAATTTGATAGGCTTTATGCAAACAACTCTCCCTCTGCTGGAGTCTGG
CAAGTTTGCTTTTCACTGGACGCTAATTCAAGTGCCATACAAAACTAAAA
TAANAGTTTTACTTATAACACA
SEQ ID NO: 208
CAGAAATCGCAATTGAAGACCAGATTTGTCAAGGTTTGAAACTGACATTT
GATACTACCTTCTCACCAAACACAGGAAAGAAAAGTGGTAAAATCAAGTC
TTCTTACAAGAGGGAGTGTATAAACCTTGGTTGTGATGTTGACTTTGATT
TTGCTGGACCTGCAATCCATGGTTCAGCTGTCTTTGGTTATGAGGGCTGG
CTTGCTGGCTACCAGATGACCTTTGACAGTGCCAAATCAAAGCTGACAAG
GAATAACTTTGCAGTGGGCTACAGGACTGGGGACTTCCAGCTACACACTA
ATGTCAATGATGGGACAGAATTTGGAGGATCAATTTATCAGAAAGTTTGT
GAAGATCTTGACACTTCAGTAAACCTTGCTTGGACATCAGGTACCAACTG
CACTCGTTTTGGCATTGCAGCTAAATATCAGTTGGATCCCACTGCTTCCA
TTTCTGCAAAAGTCAACAACTCTAGCTTAATTGGAGTAGGCTATACTCAG
ACTCTGAGGCCTGGTGTGAAGCTTACACTCTCTGCTCTGGTAGATGGGAA
GAGCATTAATGCTGGAGGCCACAAGGTTGGGCTCG
nt: 624
SEQ ID NO: 209
GACACACGAGCATATTTCACCTCCGCTACCATAATCATCGCTATCCCCAC
CGGCGTCAAAGTATTTAGCTGACTCGCCACACTCCACGGAAGCAATATGA
AATGATCTGCTGCAGTGCTCTGAGCCCTAGGATTCATCTTTCTTTTCACC
GTAGGTGGCCTGACTGGCATTGTATTAGCAAACTCATCACTAGACATCGT
ACTACACGACACGTACTACGTTGTAGCCCACTTCCACTATGTCCTATCAA
TAGGAGCTGTATTTGCCATCATAGGAGGCTTCATTCACTGATTTCCCCTA
TTCTCAGGCTACACCCTAGACCAAACCTACGCCAAAATCCATTTCACTAT
CATATTCATCGGCGTAAATCTAACTTTCTTCCCACAACACTTTCTCGGCC
TATCCGGAATGCCCCGACGTTACTCGGACTACCCCGATGCATACACCACA
TGAAACATCCTATCATCTGTAGGCTCATTCATTTCTCTAACAGCAGTAAT
ATTAATAATTTTCATGATTTGAGAAGCCTTCGCTTCGAAGCGAAAAGTCC
TAATAGTAGAAGAACCCTCCATAAACCTGGAGTGACTATATGGATGCCCC
CCACCCTACCACACATTCGAAGAA
nt: 338
SEQ ID NO: 210
ACCTGAGGCCTCGGTGGGGCCAGTGCGACGCTGGCTTAAGGAGCTGGAGG
GGTTCCTAATACACATTTAATTCAGTTTCTCTTCCCTAAGAGGCTGCCGG
AGTTGGGGCCTCCTCCAGCAGAGACCCTCGGACCCCTGCAGGGCCTGGAC
TTGGGGTGAACAGGGCTTCAGTCAGCGCAAGTATTCCATTTGCATTTGGT
AATTTTTCATGCCACCTATTTATGAATATATAAATCTTTATACCAAATCT
ATTTTTTAAAACATGGAAAAGTTGCCTTTATGGAAACTTGGCAGAGCCAG
AGTGTACACATTCCTAAACCATTAAACAGATTTCTATA
nt: 556
SEQ ID NO: 211
GGATAATGATACCTCTGACCTTTCTTCCTTTTGGGAAGTACTTGAGTGTG
CAGCTGCATGAGGCCTCAGCAGGAGAGAGATTTTAGGTCCAAGAAGCTAT
ACCAGTAGGACAAGGCAGGAAAATACTACACTTTCAGGATCAAGCCCCTC
TGACTCTCATTTGGAAACTGGATGTTTGCTAAGCACCTGCTTCTTAAGGA
TGCCGAGGGATTTAATGATACTCCCAGAAACCTGGAGAGATTAATGGGGC
CTATGGAGAAGTGCTCTGAACTCAGTGTTGGGACTTGAATAAAATTAACC
ATTGTCATGTTTTCAGAACAACTAAGCTGTTTTATATTTCATGTGCATGA
AAGCCCTAGAACTAAGTTGTGTTATTTCCAGAAATGAAATAGATCCCACA
GTTAGATGATGTGGCCATTAGGAAGTACCAAATTTATAAAAATCACTGGA
GGTCTGTCTGAGCAGTACCTAATAAAATATAGTATACTGAAAGTGAACAG
ATCTTTGTCTCTTTCTTTGGCTGCTTGATACTTTATCTGTGTCTGCCGGA
CAGTGC
SEQ ID NO: 212
CAATAAAAGCAGGTTAACCTCAATGATAGCAGTTAAAATGTTCTATCTTA
TGTATTTCTTTTAAGTATTACCATTATGGTGCTACTGAGCGTTTTCTTTT
GGTAAAAAGAAAAATGCCATGGGCTGCAGTCTTCTTCCATCACTTTTCCC
TACCAGGTCCATTAATATGCTTATAACACTAGTGCCAGTTATTTTATTTG
ATAATGCTTATGGTATTTGTATATTTGTTTGCATTCCAATTTTGTTTAAT
AATGAGTGTGTAAACTGCATACGTTAAATAAATGTAAATACTAATGTACT
GCTGC
SEQ ID NO: 213
TTTTATTACCCAAGTTTTAACCTCTGTCTGGTGATTTGTTGTTGTTGTTG
TTGTNGTTGTTGTTGAAGTTCAGGCTGCATGTGGGATAGGTTTGCTCAGG
CATACTTCTTAGGAAGTAGTCACTTGCATGACTGTTTTTGGGATAACTCT
TTGAGTATTTGGAGAGGTCTATTGTAACTTCTGAAAGGCATTGTTTTTAC
GTATGAATGTTCTAAAATTCATTCTAAATGGTCATGAAAAGAAAAGGATT
CACATTTTAGAATGGCAATAGTCCCTGAGGACTATTATGTCTTTTAGATT
TCCTGTGGGTTTCTAGGAATGTTAGTGTAACTTANATTTCCACCTACCTG
ATTTCTGGATGTGCCTATTGGAACTTGCTGAGATCTTTTTTTTTCCTTAA
CATGTTGTCCCCTTGACCCGTACTTCGAAACTAAACATATTATTTTATTT
GCTTACACTTCAGGAGGCAATTGGCAGACACCAGGCCAACAGTCT
SEQ ID NO: 214
GCTCTGACCCCAGTTGGAAATGTATCTGTACTTTGTCCGGCTTCCACTCA
AGGACCATTTATGACATTGCTTGGTGTCAGCTGACAGGGGCTCTGGCCAC
AGCTTGTGGGGATGACGCGATCCGCGTGTTTCAGGAGGATCCCAACTCGG
ATCCACAGCAGCCCACCTTCTCCCTGACAGCCCACTTGCATCAGGCCCAT
TCCCAGGATGTCAACTGTGTGGCCTGGAACCCCAAGGAGCCAGGGCTACT
GGCCTCCTGCAGTGATGATGGGGAGGTGGCCTTCTGGAAGTATCAGCGGC
CTGAAGGCCTCTGAGCTACCTCGACTTTGGACAGAGTAATGACTCCCCAG
AAAACGTCATATAAGACTTTACCAGCCCCTGAGAGGACCAGGAGGAGCAT
CCTTGACCTTCATTTAACTTGGCTCACTTCTCTTCANACTTGGGTAGAAG
TGCAGAGCCACAAAATTGCTTTCCTTCCCCGCCTTTGACATGAGGCCTTC
AGTAAAG
SEQ ID NO: 215
TGCAGGATCCGTCGACT
nt: 576
SEQ ID NO: 216
GAGAAATATAAGATTATGTATAGATCAAATCTACCTCTATTTGGTGTCCT
GAAAGAGATGAGGAGAATGGGACAAACTTGGAAAGCTTATTTCAAGATAA
CATTCCTGAGAACTTCCCCAATCTTGCTAGAGAGGCCAACATTAAAATTC
AGTAAATGCTGAAAACTCCAGTAAGATATTTCTTAAGAAAATTATTCCCA
AGATATATACTCATCAAATTATCTAAGGTCAAATGAAGGAAAAAATTTTA
TAGGCAGCTAGAGAGAAATGTCAGGTCACCTACAAAGAGAATGGCATAAG
ACAAAAAGTAGAACTCCCAGCAGAAACTCTAAAAGCCAGAAGAGATTAGG
GGCCAATATTTAACATTCTGAAAGAAATTCCAACAAGGAATTTCATATCC
AGCCAAACTAAGCTTCATAATTGAAGGAGAAATAAGATATTTTCCAGACA
AGCAAATGCTGATGAAATCCATCACCACCAGACCTGCCTTATAAGAGCTC
CTGAGGGAAGCACTAAATATTGAAAGGGAAGAACTTTATGAACCATTTCA
AAAACACATTTAAGTNCACAAAGCAG
nt: 341
SEQ ID NO: 217
CCTTATTTTACAGGTGAAAAACCACGAATCAGATAGATTTTTATTTGCCC
AAGTCACATAATATTAAGAACAGGCCAAGTGTGGTGGCTCATGTCTGTAA
TCTGAGCACTTTGGGAGGCTAAGGCGGGTGGATTTCCTGAGCCTAGGAGT
TTGAGATCAGCCTGGGCAACATGGCGAAACCTCATCTCTACAAAACATAC
AAAAATTAGTCAGTGTGGTGGTGAGAGCCTGTAGTCCTGGCTACTCGTGA
GGCTGAGGTGGGAGCATCACCTGAGCCTGGGAAGTCGAGGCTGCAGTGGC
AACAGAATGGGTAACCTGGACATCAGAGTGAGACCCTGTCT
SEQ ID NO: 218
CTCACACCTGTAATTCCATTACTTTGGAAGGCTGAGAGAGGAGGATCAGT
GGAGCCCAGGAGTTTGAGACCAGCCTGGGCAATATAGGGAGACCCTGTCT
CTACAAAAATGAAATAGCCAGGCGAGGTGGCATGTGCCTGTGGTCCCAGC
TACTTGGGAGACTGAGGTGGAAGGCTGCCTTGAGCCCAGGAGTTCCAGGC
TGCAGTGAGCCATCATTATGCCACTGCACTCCAACCTGGGAGACAGAGTG
AGAGAGACCCTGTCTCAAACAAACAAACCCAAAATAGGCCAGGCACAGTG
ACTCATGCCTGTAATCCCAGCACTTTGGGAGGCTGAAATAGGCGGATCAT
TTGAGGTCAGGAGTTCAAATTCAAGACCAGCCCGGCCAACATGGCAAAAC
CACATCTCTACTACAAATAAAAAATTAGTTGGGTGTGGNGGAGCATTCCT
GTAATCACAGCTATTCAGGAGGCTGAGGCATGANAACCGCTTCA
nt: 379
SEQ ID NO: 219
TAAATTTAAAACATTTTAATTAGCTGGCATGATGGCATGCACCTGTAGTC
CTACCTACTTGGGAGGCCAAGGCAGGAAGATTGCTTGAGCCCAGGAGTTT
GAGCTTACTGTGAGCTGTGATCACACCACTGCACTCCAGCCTGGGTGACA
AAGGAAGACCGTATTTCTAAAAAATAAAAAATACAAATACAACTACAAAC
TAGCACTAGACCAACAGTGACTATGTACCATGAACTGAGGAATATTATTA
ATTCCACCATTTGCATCTGAGGTTAACAATATGTCAATGACTTAAATAAC
ATCATATCTCTGAGAGTAATTTCTCCTATATTTCCATGACAAATGTTAGA
TAATTTTCCATTTTTTCCATTCAACAAAA
SEQ ID NO: 220
TTTTCAGGCATGTCAGAGAAGGGAGGACTCACTAGAATTAGCAAACAAAA
CCACCCTGACATCCTCCTTCAGGAACACGGGGAGCAGAGGCCAAAGCACT
AAGGGGAGGGCGCATACCCGAGACGATTGTATGAAGAAAATATGGAGGAA
CTGTTACATGTTCGGTACTAAGTCATTTTCAGGGGATTGAAAGACTATTG
CTGGATTTCATGATGCTGACTGGCGTTAGCTGATTAACCCATGTAAATAG
GCACTTAAATAGAAGCAGGAAAGGGAGACAAAGACTGGCTTCTGGACTTC
CTCCCTGATCCCCACTCTTACTCATCACCTGCAGTGGCCAGAATTAGGGA
CTCAGAATCAAACCAGTGTAAGGCAGTGCTGGCTGCCATTGCCTGGTCAC
ATTGAAATTGGTGGCTTCATT
nt: 598
SEQ ID NO: 221
GATTAACTTTCATTTTAAGCTCTTCTCTACTAATTCTGTTCGTATGTTTA
TTCATTTTGCGTTGATCATATTTTGTACACCAGGCACTCTTCTCAGTTTT
ATATGTGTGTTAATTTACTCCTTTCAAGAGCCCTATGATACATGAATTTA
TCTCCATTTTATAGATGAGGAAATTAAGACCTAGAGTTACTGAACTTGCC
CAAGGTTATACAGCTGATGGGTAGGGCCAGAACTTTGCCTCAGAGAATCT
GAATTTCCAAAAAATAACCTAAAAGAGAAATTTAAGTACTAATTAGTAAG
CAAAGAAATGCACATTTAAGGAAGACAGTGCACATTTAAGGAAGACAGTA
ACCTTTTATCTATTAGAGAAAAACACACATTCTGTCTTTAACACACACAT
AAATCTTATATTGGCAGGGATTTTCTTTATTCAGCAATTATTTATTGGTT
GTCTGCTTTGTGGTACACATAAATGCTGGGGATAAACACTTAATAAAATA
TACTTCCTTCTCTTGAATATCTTGCACTTTAAGTGGGAAGGTAAGTCAAC
AGAGTAGAGGTGATATATCCAAGTGATAGACTGTTTCATTGCCAGTAG
SEQ ID NO: 222
GTTGCCTGAGAGTGACCTTTGCATCTGCCTGTCCAGCCAGCATGGAACCA
AAGCGGATCAGAGAGGGCTACCTTGTGAAGAAGGGGAGCGTGTTCAATAC
GTGGAAACCCATGTGGGTTGTATTGTTAGAAGATGGAATTGAATTCTATA
AGAAGAAAAGTGACAACAGCCCCAAAGGAATGATCCCGCTGAAAGGGAGC
ACTCTGACTAGCCCTTGTCAAGACTTTGGCAAAAGGATGTTTGTGTTTAA
GATCACTATGACCAAACAGCAGGACCACTTCTTCCAGGCAGCCTTCCTGG
AGGAGAGAGATGCCTGGGTTCGGGATATCAATAAGGCCATTAAATGCATT
GAAGGAGGCCAGAAATTTGCCAGGAAATCTACCAGGAGGTCCATTCGACT
GCCAGAAACCATTGACTTAGGTGCCTTATATTTGTCCATGAAAGACACTG
AAAAAGGAATAAAAGAACTGAAT
SEQ ID NO: 223
TGGTACTGAACCTACGAGTACACCGACTACGGCGGACTAATCTTCAACTC
CTACATACTTCCCCCATTATTCCTAGAACCAGGCGACCTGCGACTCCTTG
ACGTTGACAATCGAGTAGTACTCCCGATTGAAGCCCCCATTCGTATAATA
ATTACATCACAAGACGTCTTGCACTCATGAGCTGTCCCCACATTAGGCTT
AAAAACAGATGCAATTCCCGGACGTCTAAACCAAACCACTTTCACCGCTA
CACGACCGGGGGTATACTACGGTCAATGCTCTGAAATCTGTGGAGCAAAC
CACAGTTTCATGCCCATCGTCCTAGAATTAATTCCCCTAAAAATCTTTGA
AATAGGGCCCGTATTTACCCTATAGCACCCCCTCTACCCCCT
SEQ ID NO: 224
TTTTTCTTGTTTTTGTGTGTCTACCTTGGCATATACTAAAGGAAGGTGTG
TATTCATTTATTACATGATATCTCTGGGTTATAATTATTTACATATATGA
ATTTGAAAGAAAGATTGAGAGGGATATGTGTGACCTTTGTTTCATTATGA
TCATTTACATGACTAAAGATAAAGATCATATGTCTGATTTTCAGTTTAAT
GGCAAGTTACTTAAAATAAATGAAATATGTTTTTATTGTTTTCGTGGGTT
TGATGCTTTGTGTTTTATTTCAAGTAACTTGAGAATGCATTGTGTTTGGT
ACTGTTTTTTATGAATATCATTAAAAATTTATTTAAGGAGAGAGTAATTT
TGCAATAATATTTTTGATTTATTTGAAAATAAAATTCAAGATAAATGAAA
TAATTGAAATTTTCTAAAGAAGGAATTGAATATATTTTTACATTTGAATG
AACTAAGGATTAACTGAACCATTTATATATAGTACTTTCAGAACTGAATG
TCTTAAATGATAAAGCTCTAATTGGTTAAAGTGACTTTCTTTCAAGTCAA
AGAACCCAGAAACTGAATAGATGATCTAACTACTGCCACTGAGGTTTTGG
ATTAGTGAGTATAAATTT
SEQ ID NO: 225
TGCAGGATCCGTCGACT
SEQ ID NO: 226
GACAATCAGAGCAGATCTTGGGCTTCTGTGGCTCATCTCAGCCCTTTATA
ACTGGCCTGAGAAGAGGGTTTATCTACTTGTGCAAGTGGCCCAGAAATCT
CACTCGTACATGAGGCTTTGGAACATCCTTGCAAAGGTACGCTGAAAGCA
AATTGCTGTTTTCCTGGTGGTTCTGCACGTTTCCTAACTTTTATCATAGT
TTGATTTTCATTATTTAAGAAAAAATAAAAAATCCAAAGACCATAAGATG
GCATTAGATTTTTTACCATTAAATTATTAATGCCTATTTGGTGCTCATAA
AGATTAATCATGTCACGCATGTTTCCAATCTTTCTTTTGCAGTATATTAT
TTTCTAAAAATTGTTACATGCAAATTTAAACCAAGATTTATCAGTA
SEQ ID NO: 227
TTGGAAGAAATAAACCAAGGCAGAAAAATTTTAAATGGCCAAAATAAATT
GTATTGCTAACTTAGATGGCCACAGATGGGGGCAGGGGTGGAGAGAGGAG
AAATTGAAAACNCCACAAAGACCCCGCAATGGCTAGAACTTGAAATCTCT
GGATATTGCAACAATAGCAGCCTCCTTAAGTCAGCAAAAAGATAAAGATT
GATCCAATGTTCTATATTACAGAACAGAGCAGATTGTCAATATAGCAAAT
AAAGTTACCGTTGAGTGGACTGCGCTGTNTAAGCTGCTTGGTTGGCCTTA
AGTGCCGACAATTAAGAGATGAAGGCAATGAGAACTGAAACAAACATTTA
AGTTCAAGACCCAGTTTACTGACACTGGGACTATTACTATATCTCTTTGG
GCCTCAGTTTACTTATCTGTAACATTAAGAGGTTGGATTACATGATGTCT
CACGATTCTTTTTTTTTATTTAGAGATGGGGTTTTGCTCTGTTGCCCAGG
CTGGAGTGCAGTGGCATGATCATAGCTCACAGCAG
SEQ ID NO: 228
CCAGCCTGTCACTGGCCTGGCCAAGGAGGAGAGACAGGCCAGGGATTCTG
GTCCTAACTCTACTGGCCACACTGTGTGGCCTGAGACCCCCCTTTCCCTC
CCAAGCCCCTGCCTCCGCATCTGCGTGGTGAAGGCCATTGGCCCTCATCG
GTGGATCTGCGTTTCCTCGGGCCTACACTGTCTAGGATTGTGCGGGGCTG
GTGAGAGAACAAGATCTCTTCCGTGTTCAAGGCAGACTTCCTGCCCCCTG
CACCCTGCTCTCTCCCAGGCCTTGAGGTCAGTGTGAGCCCCAAGGGCAAG
AACACTTCTGGAAGGGAGAGTGGATTTGGCTGGGCCATCTGGATGGAAGG
TAAAAAAAAGAAAATCCCTTGAAAGGAGATTGAGGGAAGTTT
nt: 419
SEQ ID NO: 229
AAGAGAAAGGACTCAGTGTGTGATCCGGTTTCTTTTTGCTCGCCCCTGTT
TTTTGTAGAATCTCTTCATGCTTGACATACCTACCAGTATTATTCCCGAC
GACACATATACATATGAGAATATACCTTATTTATTTTTGTGTAGGTGTCT
GCCTTCACAAATGTCATTGTCTACTCCTAGAAGAACCAAATACCTCAATT
TTTGTTTTTGAGTACTGTACTATCCTGTAAATATATCTTAAGCAGGTTTG
TTTTCAGCACTGATGGAAAATACCAGTGTTGGGTTTTTTTTTAGTTGCCA
ACAGTTGTATGTTTGCTGATTATTTATGACCTGAAATAATATATTTCTTC
TTCTAAGAAGACATTTTGTTACATAAGGATGACTTTTTTATACAATGGGA
ATAAATTATGGCATTTTTT
SEQ ID NO: 230
CTGAGAGTCACTGTGTTTTTAGCCAAATCTAAGGGAGAAAATGAATATTG
ATAGCAGCATGCTGTAGCCAGCTCCTTAAAGGAAGGATGGTGCCTGGTAC
AGAGTTAGAGTTAGTGCTTCAGTAAATAATGAATGTGTGCTAGGTAGGTT
CTGCTGGGTAGGCTGCATGCATTGACCAATTTATTCCTCCTTGTTTCAAA
ACAGGATTTAAGGGCACTTATATATATATATTTTTTAGTTTTTTTAATGT
AAATGAGAGAATAAAGATATATATATATGTCTATATATGTATATATGTAT
ATATATGTCTATATGTCTATATGTATATATGTCTATATGTATATATGTGT
GTGTGTATATATATATATATATATATAAGTTTTCTGTTGCTAGCATAACA
AACTACCAGAAACTTAGCAACTGAAACAACATGAATTTATCTTACGGTTC
TATAGTTCAGAAGTCTAACGTGTCACTGGGATGAAATCCAGGTTTCAACA
GGACTGGGTTCCCTTCTAGCTCATTCAGCTACCTGGCTCATTCAGGTTGT
NGGCAGAATATACTTCCATGAAACTGTAGGGCTGAGACCCCGTTCCTTCC
TGGCTATCATCTGAAAACTTTC
SEQ ID NO: 231
AGGCGCAGCCCAGCCTCGAAATGCAGAACGACGCCGGCGAGTTCGTGGAC
CTGTACGTGCCGCGGAAATGCTCCGCTAGCAATCGCATCATCGGTGCCAA
GGACCACGCATCCATCCAGATGAACGTGGCCGAGGTTGACAAGGTCACAG
GCAGGTTTAATGGCCAGTTTAAAACTTATGCTATCTGCGGGGCCATTCGT
AGGATGGGTGAGTCAGATGATTCCATTCTCCGATTGGCCAAGGCCGATGG
CATCGTCTCAAAGAACTTTTGACTGGAGAGAATCACAGATGTGGAATATT
TGTCATAAATAAATAATGAAAACCTAAAAAAAAAAAAAAAAAAAAAAAAA
SEQ ID NO: 232
TNCACTCACACACTCCCAAACCTTAACAAACACATACATGTGCAGCCAAC
CCAATGGGCCAGCCTCTTTTATGCTCCTCACATGTTTCCTTTAACTGGAA
TACCCATGACAGCTCCCTACATAGTTACTTGTAAACTCCTCCTCTCTGTA
TAAGTTTTCCTGAATTTTTTTGATAAAATTAAGTTGTGCCACCCCTTTAT
GCTCTCTTANAACTTTGTTCTGTTCTCATGGCTGTTCTGCAACGAATCTC
ATTGTGTTCTCCTACTCAATTACATTCCTGCGTCTCCCACTAGATGGCAG
ACTCTTTGAGAGTAGGAGATTCCCTTGTTATCTCTGGATCCCTGGCACTT
GCAGAAAGCCTGTTACGTAATAATTGCTCAACAATTAGTTTTTAAATAAA
TGAATTATTTTTAAAACGCCAAAATTACAATGATTGTGCATTAAGTGAAA
GATGACCATCTAAAAACATAAAGCCATGCTTCATGACATTGGC
SEQ ID NO: 233
GACCATTCAGGGAAATTTTATAAAAAATGCAGATACTGTCTTGAGCAGAT
CGAAATGCCGATGAGGTGGATGCAATTTCCTTTTGTGCAAGCAGTGCACG
GTGCCCCCCCCTCGGGTGTCCGTGCTGTGCCTTAGCTTCCCCAGGTGCCG
GGACTCACACCTGCTAGGGGCTGGGCAAGGCCCCGGCTCTGCTTTCTCTG
AAGGGCTTGTCCAAGTTCATTGCCCTGTTACAGGTGGTCAAGACGTCCGG
CCGCCTTGACCCAGGCTACCCTTAGCCAATATCCTCTGCCCCTGGGTGGT
TGGTGGCTGGGCCTCAGGGTGGGCAACGTTAGGGGTTTGGCGAAAGCCCG
CCCCATGGGATTGAGGGACGGGGCTGCACTCCAACCGTCTGCACCTGCTC
TTCCCCCACCCCTGTGGGACCTCATCTTCACGTGCCATGTGTGCTGAAGG
CCCAGGGCCCAGCAGGGGGCAGTGGCACCTGTTGACGGAAAAGCCGAGGT
GCTTACCAATGGACCTTCTGGCCCGCCCTCCCCTGTACTTGTCGGGCATT
CAGGGCCCCGACCTGTGCCTACCCGCA
SEQ ID NO: 234
CAACTGTGTTCACTAGCAACCTCAAACAGACACCATGGTGCACCTGACTC
CTGAGGAGAAGTCTGCCGTTACTGCCCTGTGGGGCAAGGTGAACGTGGAT
GAAGTTGGTGGTGAGGCCCTGGGCAGGCTGCTGGTGGTCTACCCTTGGAC
CCAGAGGTTCTTTGAGTCCTTTGGGGATCTGTCCACTCCTGATGCTGTTA
TGGGCAACCCTAAGGTGAAGGCTCATGGCAAGAAAGTGCTCGGTGCCTTT
AGTGATGGCCTGGCTCACCTGGACAACCTCAAGGGCACCTTTGCCACACT
GAGTGAGCTGCACTGTGACAAGCTGCACGTGGATCCTGAGAACTTCAGGC
TCCTGGGCAACGTGCTGGTCTGTGTGCTGGCCCATCACTTTGGCAAAGAA
TTCACCCCACCAGTGCAGGCTGCCTATCANAAAGTGGTGGCTGGTGTGGG
CTAATGCCTGGCCCCACAAGTATCACTAAGCTCGCTTTCTTGCTGTCCAA
TTTCTATTAAAGGTTCCTTTGTTCCCTAAGTCCAACTACTAAACTGGGGG
ATATTATGAAGGGCCTTG
nt: 511
SEQ ID NO: 235
TTTTTTAATTTCACCAAAATTTGTTGACGTCCCTTGATTTGCTGATAGGG
ACAATAATTAAATATTTTCCACTTGTTTTTATAAAAACTGTAATGGTGAT
TTGTTTAACAGATGTTGACTTAGCACCTTCTCTCTTTTTTTTTTTTTTTT
TTTGAGTTGGAGTCTTGCTCTGTCACCCAGCTGGAGTGCAGTGGCACGAT
TTCGGCTCACTGCAACCTCCGCCTCCCAGGTTCGGGCGCTTCTCCTGCCT
CAGCCTCCCANATAGTTGGGATTACAGGTGCATGCCGCCACNCCTAGCTA
ATGTTTTTTGTATCTTGGTANANATGGNGTTTCACCTTGTTGCCCATGCC
GCTCTTGAACTCCTTGGCCTCCCAAAGTGTTAGGATTACAGGCGTGAGCC
ACTGTGCCTGGCCCCAATTTANCACCTTACTGGGTGCTGAGGCTGTGAGC
CATAGTAGAATGCATGTGATCCAGGGCCTTGCTGAATTCATGGGCTAATA
GGGAGCCTGAC
nt: 592
SEQ ID NO: 236
TGAGCGTTGGGCTGTAGGTCGCTGTGCTGTGTGATCCCCCAGAGCCATGC
CCGAGATAGTGGATACCTGTTCGTTGGCCTCTCCGGCTTCCGTCTGCCGG
ACCAAGCACCTGCACCTGCGCTGCAGCGTCGACTTTACTCGCCGGACGCT
GACCGGGACTGCTGCTCTCACGGTCCAGTCTCAGGAGGACAATCTGCGCA
GCCTGGTTTTGGATACAAAGGACCTTACAATAGAAAAAGTAGTGATCAAT
GGACAAGAAGTCAAATATGCTCTTGGAGAAAGACAAAGTTACAAGGGATC
GCCAATGGAAATCTCTCTTCCTATCGCTTTGAGCAAAAATCAAGAAATTG
TTATAGAAATTTCTTTTGAGACCTCTCCAAAATCTTCTGCTCTCCAGTGG
CTCACTCCTGAACAGACTTCTGGGAAGGAACACCCATATCTCTTTAGTCA
GTGCCAGGCCATCCACTGCAGAGCAATCCTTCCTTGTCAGGACACTCCTT
CTGNGAAATTAACCTATACTGCAGAGGTGTCTGTCCCTAAAGAACTGGTG
GCACTTATGAGTGCTATTCGTGATGGAGAAACACCTGACCCA
nt: 572
SEQ ID NO: 237
CTTANAAGAGTTGCTCATTCACACCCACGCCCTTGCCCAAGGCTGGCCCA
CTCAGAGCGAAACTTAACTTTTGTCTGGATGGGAAGAGAAGTAAGTCTAC
CCCGAGGTTGCCATGTTGAAGAGTGAGAGGTCCAAGTGATTCTGTGCATT
GAAACCAAGACACCCCACCCAGAACACTTCTTCCCTCCCTCAGCCCAAAC
CAAAGGCTGGGGTTCTCATCTCCAAGTGGCTGTTCTCCAACTTTCCCAAG
CCGCTTGCATTCCCCAGACTGGACTACTGTGGCGGTTAGGTTAGATTTGA
AGACGGGGCCCAGGCTGGGTATGAACGGGTGCAGCCCTCTTCTCCTCTTC
CCCCCCACATCTCTCATGAGAGAGGTAGTGGCATTTCCTTCTCAGGGAGC
TTCAATGGGAAAGGTCTCGAAAGCTTCAGGAGGAGCAGAATACCAACGCA
GGGGGATGGCTGTAACGATCTCACCGTCTCCTAACCTCAGTCCCTTTTTT
GAGAGTGAATGGTGGAGGGTGGGAAAGGGACCCAAATTTGTAGATCTCTT
TGTCTGGGGGAGGGGAANGATG
nt: 482
SEQ ID NO: 238
TTAAAACAGGCGCAGGGGTAAAAATGAGAATGAATCTGAAAAAAGAGAGT
TGGTGTTTAAAGAGGATGGACAAGAGTATGCTCAGGTAATCAAAATGTTG
GGAAATGGACGATTGGAAGCATTGTGTTTTGATGGTGTAAAGAGGTTATG
CCATATCAGAGGGAAATTGAGAAAAAAGGTTTGGATAAATACATCAGACA
TTATATTGGTTGGTCTACGGGACTATCAGGATAACAAAGCTGATGTAATT
TTAAAGTACAATGCAGATGAAGCTAGAAGCCTGAAGGCATATGGCGAGCT
TCCAGAACATGCTAAAATCAATGAAACAGACACATTTGGTCCTGGAGATG
ATGATGAAATCCAGTTTGACGATATTGGAGATGATGATGAAGACATTGAT
GATATCTAAATTGAACCAAGTGTTTTTACATGACAAGTTCTCTGAGGATG
GTTCTACAGTTGGGATTTTGGCCATCATCAAC
nt: 545
SEQ ID NO: 239
TTTGAAGGCAAAGAGGGATTAATCTGTGCTGGCATCATGTAAGGAGACTT
GATAGATAAGAAAAAGCTTTACCTAAGTTTTGAAGAATAGGTTTTTCATA
ATGGAAAATTTAAGGGAAAAATCTCCAAAAAAGTGCTACTCAAGTTTTAT
CCATTTGTATTTCCAACACAGCCTAGGACAGTACCTGCACATAGTAGGTG
ATTAATAAAAATTTAGAAAGCATTAATACTAAAGAGGAAAAATAGCAATG
GCAAGAAAACACATGTAGGGAACACATGTAGCCAAAAAATAATATATAAT
CAGAGAAATAATAGGACTTCTGGAAAAAAAAGATGAGATCAGATTGGTTA
GGATCTTTACTAACATGACAAGAGCATGAATTTTTTTTCTGTAGATAATA
AGTATGAAAGAATTTTAGCTTAAAAATTAGCATAATTTGGATCCACATAT
GCAAATCAATGAATGTAATTCATAATATAAACAGAACTAAACACAAAAAC
CACGTGATTATCTCAATAGACACAGAAAAGGCCTTCAAAAAAATT
nt: 624
SEQ ID NO: 240
GACACACGAGCATATTTCACCTCCGCTACCATAATCATCGCTATCCCCAC
CGGCGTCAAAGTATTTAGCTGACTCGCCACACTCCACGGAAGCAATATGA
AATGATCTGCTGCAGTGCTCTGAGCCCTAGGATTCATCTTTCTTTTCACC
GTAGGTGGCCTGACTGGCATTGTATTAGCAAACTCATCACTAGACATCGT
ACTACACGACACGTACTACGTTGTAGCCCACTTCCACTATGTCCTATCAA
TAGGAGCTGTATTTGCCATCATAGGAGGCTTCATTCACTGATTTCCCCTA
TTCTCAGGCTACACCCTAGACCAAACCTACGCCAAAATCCATTTCACTAT
CATATTCATCGGCGTAAATCTAACTTTCTTCCCACAACACTTTCTCGGCC
TATCCGGAATGCCCCGACGTTACTCGGACTACCCCGATGCATACACCACA
TGAAACATCCTATCATCTGTAGGCTCATTCATTTCTCTAACAGCAGTAAT
ATTAATAATTTTCATGATTTGAGAAGCCTTCGCTTCGAAGCGAAAAGTCC
TAATAGTAGAAGAACCCTCCATAAACCTGGAGTGACTATATGGATGCCCC
CCACCCTACCACACATTCGAAGAA
SEQ ID NO: 241
CAAGATGACAAAGAAAAGAAGGAACAATGGTCGTGCCAAAAAGGGCCGCG
GCCACGTGCAGCCTATTCGCTGCACTAACTGTGCCCGATGCGTGCCCAAG
GACAAGGCCATTAAGAAATTCGTCATTCGAAACATAGTGGAGGCCGCAGC
AGTCAGGGACATTTCTGAAGCGAGCGTCTTCGATGCCTATGTGCTTCCCA
AGCTGTATGTGAAGCTACATTACTGTGTGAGTTGTGCAATTCACAGCAAA
GTAGTCAGGAATCGATCTCGTGAAGCCCGCAAGGACCGAACACCCCCACC
CCGATTTAGACCTGCGGGTGCTGCCCCACGTCCCCCACCAAAGCCCATGT
AAGGAGCTGAGTTCTTAAAGACTGAAGACAGGCTATTCTCTGGAGAAAAA
TAAAATGGAAATTGTACTTAA
SEQ ID NO: 242
TGCTTGGCCCTCTACCTCCTGCCCTCTTCCTGTTCATCTCCCAACCACTG
CACTCTTGATTTTTATACCACACAGAAGGTAAGAAAATTCTAGGAACCCT
AAGGATCAATCCTCTCCATTTTCACTCAAATGCCTGGGGCCCAGCTCTGC
AATGACTGACTCCAGGGCCTCTTTCCTCACTGCCAGCATAGAAGTCAGGG
GAGCCAGCTGGGCCCTGCGGTCAGGAAGGTTCTCATTTTTGGAGCATTCC
CTGAGCCCAGATCATAGGAGCAGCTGTCCCTGGTGGGACACAGGAGTCAT
GACTCCTACCCTCCACCCTCCACACCCACCAGGCATTTAGCAGTCTGTCC
TATGCAAGACAGATGAATTCTCAGCCAGGATACCTCAAGGCAGGCAAAGG
TGAGTGGAGGGAAAATTCACAAACATTCAGGGTGTGTGGTGCTGGCATCA
CCATGGCCAAATCCAAGAGGTCTTCCTGGAAGAGGGCCCAAACTGGAACC
AAAAGAATGCTGTCAGCAGTTGGAATAGAGCTGTGAATT
SEQ ID NO: 243
CTTTCCAAGAGGAATCCTCGGCAGATAAACTGGACTGTCCTCTACAGAAG
GAAGCACAAAAAGGGACAGTCGGAAGAAATTCAAAAGAAAAGAACCCGCC
GAGCAGTCAAATTCCAGAGGGCCATTACTGGTGCATCTCTTGCTGATATA
ATGGCCAAGAGGAATCAGAAACCTGAAGTTAGAAAGGCTCAACGAGAACA
AGCTATCAGGGCTGCTAAGGAAGCAAAAAAGGCTAAGCAAGCATCTAAAA
AGACTGCAATGGCTGCTGCTAAGGCACCTACAAAGGCAGCACCTAAGCAA
AAGATTGTGAAGCCTGTGAAAGTTTCAGCTCCCCGAGTTGGTGGAAAACG
CTAAACTGGCAGATTAGATTTTTAAATAAAGATTGGATTATAACTCT
SEQ ID NO: 244
CTTTGATAGAGAAGAAAATTCTCCTAGGATACAAGAGCCTCAACATTTTA
AAGATTTTCTGCATCTCAAAAGCGTAGGCTCCTTGCTGGGCAAGGTGAGC
CTCTGTGAGTCCTCATAGGACCGAGCAAATCTGATTCACCCCAGAAAATC
CAATATCGAAGCTGAGCTTTGGCCTGAGCGGGTTCCATTTCCTCCCCAGA
TCCTATTTAGGAAGTGTCTCCTGACAACCTCCAAAAGGTGCTAACATGCA
ACGTTCTGAAGGGTTATTGCTCAAAAACAAGATTTTCCTTGTGGTCAAGA
CTCTGCGAGCCTCGAACACGATGAATCCGCTCGAATGGGCTTGGGCTTTG
CCCGGGTGGCGCACGCTCACACGCTGGAAGCACAGCTTTGACGATCTCCA
CACACGCACAGGCACACACGCCACAGATGATGCCGGCTCATTCTCAGGGG
GTGTCTAAGTTCTGCTTTAAATATTTACCCCCTAATTGTACAAACAATAG
GGGCATGAGCCTGGTACTCGATAAATGGGGACTTNCTTAAAA
nt: 649
SEQ ID NO: 245
CTACAGCCTGGGCAGCGCGCTGCGCCCCAGCACCAGCCGCAGCCTCTACG
CCTCGTCCCCGGGCGGCGTGTATGCCACGCGCTCCTCTGCCGTGCGCCTG
CGGAGCAGCGTGCCCGGGGTGCGGCTCCTGCAGGACTCGGTGGACTTCTC
GCTGGCCGACGCCATCAACACCGAGTTCAAGAACACCCGCACCAACGAGA
AGGTGGAGCTGCAGGAGCTGAATGACCGCTTCGCCAACTACATCGACAAG
GTGCGCTTCCTGGAGCAGCAGAATAAGATCCTGCTGGCCGAGCTCGAGCA
GCTCAAGGGCCAAGGCAAGTCGCGCCTGGGGGACCTCTACGAGGAGGAGA
TGCGGGAGCTGCGCCGGCAGGTGGACCAGCTAACCAACGACAAAGCCCGC
GTCGAGGTGGAGCGCGACAACCTGGCCGAGGACATCATGCGCCTCCGGGA
GAAATTGCAGGAGGAGATGCTTCAGAGAGAGGAAGCCGAAAACACCCTGC
AATCTTTCAGACAGGAAATCCAGGAGCTGCAGGCTCAGATTCAGGAACAG
CATGTCCAAATCGATGTGGATGTTTCCAAGCCTGACCTCACGGCTGCCTT
GCGTGACGTACGTANCAATATGAAAGTGTGGCTGCCAAAAACCTTGCAG
nt: 600
SEQ ID NO: 246
GAGATGTCTCGCTCCGTGGCCTTAGCTGTGCTCGCGCTACTCTCTCTTTC
TGGCCTGGAGGCTATCCAGCGTACTCCAAAGATTCAGGTTTACTCACGTC
ATCCAGCAGAGAATGGAAAGTCAAATTTCCTGAATTGCTATGTGTCTGGG
TTTCATCCATCCGACATTGAAGTTGACTTACTGAAGAATGGAGAGAGAAT
TGAAAAAGTGGAGCATTCAGACTTGTCTTTCAGCAAGGACTGGTCTTTCT
ATCTCTTGTACTACACTGAATTCACCCCCACTGAAAAAGATGAGTATGCC
TGCCGTGTGAACCATGTGACTTTGTCACAGCCCAAGATAGTTAAGTGGGA
TCGAGACATGTAAGCAGCATCATGGAGGTTTGAAGATGCCGCATTTGGAT
TGGATGAATTCCAAATTCTGCTTGCTTGCTTTTTAATATTGATATGCTTA
TACACTTACACTTTATGCACAAAATGTAGGGTTATAATAATGTTAACATG
GACATGATCTTCTTTATAATTCTACTTTGAGTGCTGTCTCCATGTTTGAT
GTATCTGAGCAGGGTGCTCCACAGGTAGCTCTAGGAGGGCTGGCAACTTA
SEQ ID NO: 247
CGAATGTGCAGGTTTGTTACATAGGTATATATATGCCATGATGGAAATAT
TTATTTTTTTAAGCGTAATTTTGCCAAATAATAAAAACAGAAGGAAATTG
AGATTAGAGGGAGGTGTTTAAAGAGAGGTTATAGAGTAGAAGATTTGATG
CTGGAGAGGTTAAGGTGCAATAAGAATTTAGGGAGAAATGTTGTTCATTA
TTGGAGGGTAAATGATGTGGTGCCTGAGGTCTGTACGTTACCTCTTAACA
ATTTCTGTCCTTCAGATGGAAACTCTTTAACTTCTCGTAAAAGTCATATA
CCTATATAATAAAGCTACTGATTTCCAAAAA
SEQ ID NO: 248
AAAAAAAAAAAAAAAAAAAAAAAAAAAAAAAAAAAAAAAAA
nt: 425
SEQ ID NO: 249
CAAAAAAACGAAGAAAAGTGACGACAGTCTGAGGGACTTATGGGAGATCA
TCAAGTGAACCACTATATGTGTAATGTAAGTCTTGGAATGAGAAGAGAGA
AGGAGAAGGAGGAGAGAGCTTATTTGTAGAAATAATGGCTGAAAACATCC
CAAACTTTCCTTTTTTTGAGGAAAGAAATAGGCATACAAGTTCAAGAAAC
TCAAGGAACTCCAGAGAGGACAATTCTAAAGACACCCCCTCTAACATACA
TTATAATCAAATTGTCAAAAGTAAAATACAAAGAGAATCTTTTAAATTGA
CAAGAGAAAAGCAGCTGGTCACGTTCAAGGGAGTTCTATAAGAATTTCAG
CAGATTTCTCAGCAGAAACCTTGCAGGCCAACAGGCAGTGGGATGATACA
TTCAAAGTGCAAAAAAAAAAAAAAA
SEQ ID NO: 250
CGAGAGTTTACCAGTNGCCTAATAATGCAATAAAAAATGCTTTGAGATAG
CTAACNGCCCATAAAACAAACTCAAATTGCTTATAAAGTTTCTTCCCATG
TTCCCATTTGATGAAAAGTCTTACATCACATATAACTGGGAAGCAGGGGT
CCCTCCTCAATTTTCAGACATTTTGAAAGGATGACAGTTCTGTTTGTTAG
ATGAGTAAACCTCTATATTCATAAGTTCTAAAATCCTTCATTATGAGGGA
TTCAAAGTATTTATAAAAACACTGCCCTCTAAAAATTTCCTCAGATCTGA
AGTATGGNCTTGGNCCTGAATATACAGTGTTATCCTATGTTTAAAAGGGT
GATCCAGACATGAGACGCAACTAGTTGGTGCATAAGAAGGCCCCACTTGG
CTATTTCATATCTACCTACAATTGACCAAAAAAAATTTTTTAGGCCAGCA
ATTATTATTTAGCTTCGCTCTTTCTAGTGCAAGAAACTGCAGGCTGGATC
AGTAGTTCAACAGCTAAACAGTCATAAAATAGTCATTGGCATGTTAAATT
TCTTTCAATGCTTCAAAGATAAATTCCAATTCTATTTACTTATTCATTGN
GACNGNATTACTAAACAGGTAAGGATGGGAATA
nt: 251
SEQ ID NO: 251
CTTTGGGAGGCCGAGGCGGGCGGATCACTTGAGGTCAGGGGTTCGAGACC
AGTCTGGCCAACATGGTGAAACCCCAACTCTACTAAAAATACAAAAGTTA
GCCAAGTGTGGTGGCAAGTGCCTGTAATCCCAGCTACTCGGGAGGCTGAG
ACAGGAGAATCACTTTGAACCTGGGAGGCGGAGGTTGCAGTGAGCCAAGA
TCGTGCCACTGCACTTCAGCCTGGGCAACAGAGCAAGATTCCGTCCATCT
C
SEQ ID NO: 252
CTTTCTTCAGCCTTGCAGACACCTAAACATCATGTAATTACCTAAGGAAT
TCCCAAGTGCCTCTTCCAGGTTATACGTGTAAATAGCTGTTTTTATGCAA
GATTAGTTAGATACTGCTCTTTACAGGATGAGTGGTGTTGTCTTTGGCTG
GGGGGGNCTTAAATGTGTTTCTAATGTGTGTGTCAAATAATTACCTGTTA
AACAGACTGCCAATCTGGCTGAAGCCAATGCTTCTGAAGAAGATAAAATT
AAAGCAATGATGTCGCAATCTGGCCATGAATACGACCCAATCAATTACAT
GAAGAAACCTCTAGGTCCACCACCTCCATCTTACACGTGTTTCCGTTGTG
GTAAACCTGGACATTATATTAAGAATTGCCCAACAAATGGGGATAAAAAC
TTTGAATCTGGTCCTAGGATTAAAAAGAGCACTGGAATTCCCAGAAGTTT
CATGATGGAAGTGAAAGATCCTAATATGAAAGGTGCAATGCTTACCAACA
CTGGAAAATATGCAATCCAACTATAGATGCAGAAGCATATGCAATTGGGA
AGAAAGAGAAACCTCCTTNTTACCAGAGAGCCATCTTNTTTCT
SEQ ID NO: 253
GTTGTGACTCGTTGGCATGTGATCTGAAGTTCCTGCCCTGCAGCTGACGA
GCCAGTGTTTCAATAATTAAAAACAACTCAACTCACTGTCCTCCTGCCTT
GAATTTGATCATTGCGCTTTGCATGTATGTATCACAATACCACATGTACC
CCATAAATATGTACAAAGATTATGTGTCAATAAAAAACAAAAATTAAAAT
CCCAATTTTTA
SEQ ID NO: 254
GTTGCTAGTAGCGGCAGGAAGATGTCAGGCTCACTTTCCTCTGATTCCCG
AAATGGGGGGAACCTCTAACCATAAAGGAATGGTAGAACAGTCCATTCCT
CGGATCAGAGAAAAATGCAGACATGGTGTCACCTGGATTTTTTTCTGCCC
ATGAATGTTGCCAGTCAGTACCTGTCCTCCTTGTTTCTCTATTTTTGGTT
ATGAATGTTGGGGTTACCACCTGCATTTAGGGGAAAATTGTGTTCTG
SEQ ID NO: 255
GTCCCCGGGAATCGCGGCCGCGTCGACGGTTTATTTTCAGTGCTTGAAGA
TACATTCACAAATACTTGGTTTGGGAAGACACCGTTTAATTTTAAGTTAA
CTTGCATGTTGTAAATGCGTTTTATGTTTAAATAAAGAGGAAAATTTTTT
GAAAAAAAAAAAAAAAAAAAAAAAAAAAAAAAAAAAAAAAAAAAAAAAAA
AAAAAAAAAAAAAAAAAAAAAAAAAAAAAAAAAAAAAAATTTTT
SEQ ID NO: 256
TAGAGGCCTGAATAGGTAGACAATGGCAGCAGCGTTTTTAATCACAGTCC
TATTCATGCCCTAATTCGGGAGTGATGATTAAAGGACATTAGAGGGAGCA
CTTTGACATCTGATCCTTTGAACTGACGTCTGTGCAGGCTGCACTCCATA
GAGCTCACTTGGCCAAACTGATTTCCTTAAATAAAGTGCTGTGATTTCCA
ATGTAGGAAATATTACATTAGAGCCTATTGAAATGATTAGGAATTGAGGA
GCTTTTCTTTAGGTGGGAATGTGGTGTATGCTGTATACTCACAAAAGTGA
GATCATTAATATTGCATGTACTACTTTGAATATCAGGGACCACAGAGAAA
TAGCATGAGAAACGCCTTCCTGCAGTCATGCACTTAAAATGAATATGAAC
AAAAATGTGGAACTCTGCTGTCATAGCTCTCCG
SEQ ID NO: 257
GGGGCTTCTGCTGAGGGGGCAGGCGGAGCTTGAGGAAACCGCAGATAAGT
TTTTTTCTCTTTGAAAGATAGAGATTAATACAACTCTTAAAAAATATAGT
CAATAGGTTACTAAGATATTGCTTAGCGTTAAGTTTTTAACGTAATTTTA
ATAGCTTAAGATTTTAAGAGAAAATATGAAGACTTAGAAGAGTAGCATGA
GGAAGGAAAAGATAAAAGGTTTCTAAAACATGACGGAGGTTGAGATGAAG
CTTCTTCATGGAGTAAAAAATGTATTTAAAAGAAAATTGAGAGAAAGGAC
TACAGAGCCCCGAATTAATACCAATAGAAGGGCAATGCTTTTAGATTAAA
ATGAAGGTGACTTAAACAGCTTAAAGTTTA
SEQ ID NO: 258
GACCTTTGAGAAAATTAATTTAAATCCTAGAACTTTGGGTGAACCGAAGA
AATTTATAATATTTGTTTAGTTAATAACAGATAAAAAGGAAAGATTCAAG
CCTATTGGATGAGAATTTGTACATTATTTTAGAGCTAATAATAATGGTTT
TCAGTTTAGTGAGGATTTAAAAAATGTTTTTGAATCAAACTTTTTTTCTT
TATAATCCTTTTTAACTAACTCAGGAAATAAGGTATTATGAAATCCACAC
ACTGTTACCTCCTTAAAGTATGAGGATACTTCCCACTGTTTGGTCCACTA
GTGGCTGATTATTTTGTTTGTGGATTATTTGTAATTTTCTTTTTAATTCT
TCCTTAAAGAGCATGGCATTTGGAGTCACAGACCTATATTTGAATCCTGT
CATTTACTAGCGTTTTGACCTTGAACAATTATGCTCAGAGTCTCAGTTTT
TTCTTGTAAAGTGATGATGATACTACTTAACTCACAGGGTTGTAGTGAAG
ATCAAATGAGATCATGTCTGTANAACACCCTGCCCGGCACTCAATAAGTA
TTAATAGGAACCCATATACCTC
SEQ ID NO: 259
TGTTTTTATTTTTTAAAAGGTATAAACACCAAAAAAAAAATTAACATTGT
ATGAAGATGGAAAATAAGAAGATGCACTTTCTGTAACTTTGTCTAAGGAT
TTAAATTACTAACTTATGAACTCCAATTTGAATTGAACTTAACTATCGGC
TTTCTTACTGGTAAAATTATATGGTTTATTTTAAATGCGTACATATTGAC
CAATGGCCTCTGAAAAAGCACATTTTAGATACTGAAATTGAAGGAAAGAA
AATGCATCTTCAAACATTTTTTGGAATCTCACCACATATACTTTGTTANA
TTTGTGTATTGTAGGGTGTTTGTTTTGTATTTTTGTATTGTATATGAACT
TTTTTTAAATGTGACAGTTAAACACATCTTTAAAAGCATAGTCACAGACA
AAAGCATACAGTATAAAAATTTCCTTGAAAACTCCTACAATATTATATTT
GGAGGCAGCTTCAGACTGTTTTATTGG
SEQ ID NO: 260
CTCTGGCACACATTAGTTCCTCTTATATTACATTGATATAAGCAAGTCAT
ATGGATTTATCTGAGTGTAAGGAGAGCTGGAAAAAATAGTTTCTAGCAGG
TCAGCCACCTCCCAGTGAGGGCTGCATACCATAGAAGGGGAGAATGAATT
TTGGGAAAACAGGTAATTATCTCTGTCACAGAAGGGGATGAAAAGTATGG
TAGTTACNCAAGTTANACATCTGTATGGAAAATACCACTTGGTTCTACAA
ATGNGG
nt: 627
SEQ ID NO: 261
GCCTCCCGGGTTCAGGGATTTCTCCTGCCTCAGCCTCCTGAGTGGCTGCA
TTGCAGGCACCTGCCACCACGCCTTGCAAATTTTTGTGTTTTTAGTGGAG
ATGGGGTTTTGCCATGTTGGCCAGGCTGGTCTCGGACTCCTGACCTCAGG
TGATCCGCCCGCCTCAGCCTCCCAGAGGGCTGGGATTACAGGCGTGAGCC
ACTGTGCCTGGCCCCAAGTTTTGCATCTTTTAATGCCCTCTGAACAAATA
CATAGAGAAAACTCTCAGAACAATTAAAACCTGCAGAGCAACAGTGTCCT
CCATGTCTTAGGTTTCAAGTTTGCCTCTAAAATTCTAATCCATATTTTTC
TACTTCTCAGATAATTTATGTGTGTGTACTCTTCCTAGACGTACAAGAGA
CTTTTTAATGCTAAATATTTGTCAGTGCTTAACAAAAACTCAATTTCACA
TTACTCATATTGTTTTTGTTTTAATTGAATGTGAATTAAATTTTTATTAG
TTATTTGATTTGGAATGTTATGTATGCCATTAACACTATTAGGGGAATCT
CTAGCATTTCTGTATTTTTAAAGAATTTGATTCTTTTGTANATTCTGCCT
GTGTGGCATTTTAAACATGTGTGACAT
nt: 345
SEQ ID NO: 262
ACCGGCGACATGGCCAAACGTACCAAGAAAGTCGGGATCGTCGGTAAATA
CGGGACCCGCTATGGGGCCTCCCTCCGGAAAATGGTGAAGAAAATTGAAA
TCAGCCAGCACGCCAAGTACACTTGCTCTTTCTGTGGCAAAACCAAGATG
AAGAGACGAGCTGTGGGGATCTGGCACTGTGGTTCCTGCATGAAGACAGT
GGCTGGCGGTGCCTGGACGTACAATACCACTTCCGCTGTCACGGTAAAGT
CCGCCATCAGAAGACTGAAGGAGTTGAAAGACCAGTAGACGCTCCTCTAC
TCTTTGAGACATCACTGGCCTATAATAAATGGGTTAATTTATGTA
nt: 252
SEQ ID NO: 263
ATAATTCAGAACTTCTTCATATGCTCGAGTCTCCAGAGTCACTCCGTTCT
AAGGTTGATGAAGCTGTAGCTGTACTACAAGCCCACCAAGCTAAAGAGGC
TGCCCAGAAAGCAGTTAACAGTGCCACCGGTGTTCCAACTGTTTAAAATT
GATCAGGGACCATGAAAAGAAACTTGTGCTTCACCGAAGAAAAATATCTA
AACATCGAAAAACTTAAATATTATGGAAAAAAAACATTGCAAAATATAAA
AT
SEQ ID NO: 264
TTACTTTTAACCAGNGAAATTGACCTGCCCGTGAANAGGCGGGCNTGACA
CAGCAAGACGAGAAGACCCTATGGAGCTTTAATTTATTAATGCAAACGGT
ACCTAACAAACCCACAGGTCCTAAACTACCAAACCTGCATTAAAAATTTC
GGTTGGGGCGACCTCGGAGCAGAACCCAACCTCCGAGCAGTACATGCTAA
GACTTCACCAGTCAAAGCGAACTACTATACTCAATTGATCCAATAACTTG
ACCAACGGAACAAGTTACCCTAGGGATAACAGCGCAATCCTATT
SEQ ID NO: 265
GGCTGATTCCTGAGCTATAAAAGCATAATTGCTTTATATTTTGGATCATT
TTTTACTGGGGGCGGACTTGGGGGGGGTTGCATACAAAGATAACATATAT
ATCCAACTTTCTGAAATGAAATGTTTTTAGATTACTTTTTCAACTGTAAA
TAATGTACATTTAATGTCACAAGAAAAAAATGTCTTCTGCAAATTTTCTA
GTATAACAGAAATTTTTGTAGATGAAAAAAATCATTATGTTTAGAGGTCT
AATGCTATGTTTTCATATTACAGAGTGAATTTGTATTTAAACAAAAATTT
AAATTTTGGAATCCTCTAAACATTTTTGTATCTTTAATTGGTTTATTATT
AAATAAATCATATAAAAATT
SEQ ID NO: 266
CAGGAAGTCACCTGGGATTGGCTGCCTCACCCACTCACAGTGCCATCCCT
GCCCCAGGCCTCCCAGTGGCAATTCCAAACCTGGGTCCCTCCCTGAGCTC
TCTGCCTTCTGCTCTGTCTTTAATGCTACCAATGGGTATTGGGGATCGAG
GGGTGATGTGTGGGTTACCTGAAAGAAACTACACCCTACCTCCACCACCT
TACCCTCACCTGGAGAGCAGTTATTTCAGAACCATTCTACCTGGCATTTT
ATCTTATTTAGCTGACAGACCACCTCCACAGTACATCCACCCTAACTCTA
TAAATGTTGATGGTAATACAGCATTATCTATCACCAATAACCCTTCAGCA
CTA
SEQ ID NO: 267
CAGGAAGTCACCTGGGATTGGCTGCCTCACCCACTCACAGTGCCATCCCT
GCCCCAGGCCTCCCAGTGGCAATTCCAAACCTGGGTCCCTCCCTGAGCTC
TCTGCCTTCTGCTCTGTCTTTAATGCTACCAATGGGTATTGGGGATCGAG
GGGTGATGTGTGGGTTACCTGAAAGAAACTACACCCTACCTCCACCACCT
TACCCTCACCTGGAGAGCAGTTATTTCANAACCATTCTACCTGGCATTTT
ATCTTATTTAGCTGACAGACCACCTCCACAGTACATCCACCCTAACTCTA
TAAATGTTGATGGTAATACAGCATTATCTATCACCAATAACCCTTCAGCA
CTAGATCCCTATCAGTCCAATGGAAATGTTGGATTANAACCAGGCATTGT
TTCAATANACTCTCGCTCTGTGAACACACATGG
SEQ ID NO: 268
GGGTTTTCTTTCGGAAGCGCGCCTTGTGTTGGTACCCGGGAATTCGCGGC
CGCGTCGACTGCTAAACAGAATACTGCTATTTTGAGAGAGTCAAGACTCT
TTCTTAAGGGCCAAGAAAGCCACNTGNNCCCTNGGNCTAATCTGGCTGAG
TAGTCAGTTATAAAAGCCNTAATNGCTTNNTNTTTGGNNTCNTTTTTNNC
NGGGGNCGGNCTTGGGGGGGGTTGCNTCCAAAGATANCATNTNTTTCCAA
CTTTNTNAANNNAANNGTTTTAAAATCCCTTTTCCNCCNGAAAANANNGC
CCTTTAAGNGCCNCAAAAAAAAANNGTNTTCTGCANNTTTTCTANTATNA
CAAANNTTTTNGTAGAANAAAAATTTTTTTTTAGNGGCTACCCTTTNTTT
NTTANNCANNGGAGTTTNTTTTTACAAAAAAAAAANATTGGGNCCCCTCC
ACAACCTTGGGTCTNTAATNGGGGGGTTTTTAAATAAANCNTNTNTAAAT
CCCCCNNNNNNNNNCNNNNNNNNNCCNNNNNNNNNNNNNNCCCNNNNAAA
AAATTTTTNCTCCCCCNCCCTTTTTCTTCCTGCCGGCCCCAATTTAAGCC
CNGGCGCTTGGGGCAAATCCCCCTTTAGNGGGGGGGTTTANAAAAACCNG
GGGCGGGGNTTTAAAACCNCGGGGNNNGGGGAA
SEQ ID NO: 269
ACCTCTAGCATCACCAGTATTAGAGGCACCGCCTGCCCAGTGACACATG-
TTTAACGGCCGCGGTACCCTAACCGTGCAAAGGTAGCATAATCACTTGTT
CCTTAATTAGGGACCTGTNTGAATGGCTCCACNAGGGTTCACTTGTCTCT
TACTTTTAACCAGTGAAATTGACCTGCC
nt: 591
SEQ ID NO: 270
GTATAGAAAATAATGTCCCCAGNGCATAGAAAAAATGAGTCTCTGGGCCA
GTGAATACAAAACATCATGTCGAGAATCATTGGAAGATATACAGAGTTCG
TATTTCAGCTTTGTTTATCCTTCCTGTTAAGAGCCTCTGAGTTTTTAGTT
TTAAAAGGATGAAAAGCTTATGCAACATGCTCAGCAGGAGCTTCATCAAC
GATATATGTCAGATCTAAAGGTATATTTTCATTCTGTAATTATGTTACAT
AAAAGCAATGTAAATCAGAATAAATATGTTAGACCAGAATAAAATTAATT
ATATTCTGGTCTTCAAAGGACACACAGAACAGATATCAGCAGAATCACTT
AATACTTCATAGAACAAAAATCACTCAAAACCTGTTTATAACCAAAGAAT
TCATGAAAAAGAAAGCCTTTGCCATTTGTCTTAGAAAGTTATTTTTTAAA
AAAAAATCATACTTACTATTAGTATCTATGGAAGTATATGTAACAATTTT
TATGTAAAGGTCATCTTTCTGTGATAGTGAAAAAATATGTCTTTACTAAG
TTGAAATGAATACTTTCTGNCTTTGCTAATGGATAGTTATT
SEQ ID NO: 271
CTCAATTCTACTAAAAAGCCCCCCAAGAAAAGCGAATGAGAAAACAGAGT
CATCCTCTGCACAGCAAGTAGCAGTGTCACGCCTTAGCGCTTCCAGCTCC
AGCTCAGATTCCAGCTCCTCCTCTTCCTCGTCGTCGTCTTCAGACACCAG
TGATTCAGACTCAGGCTAAGGGGTCAGGCCAGATGGGGCAGGAAGGCTNC
GCAGGACCGGACCCCTAGACCACCCTGCCCCACCTGCCCCTTCCCCCTTT
GCTGTGACACTTCTTCATCTCACCCCCCCCTGCCCCCCTCTAGGAGAGCT
GGCTCTGCAGTGGGGGAGGGATGCAGGGA
SEQ ID NO: 272
GNANCNTTTCCTNTCGNAAANCGCGCCTTGTGTTGGTACCCGGGAATTCG
CGGCCGCGTCGACAAAAAAAAAAAAAAAAAAAAAAAAAAAAANTNTAGAC
TCGANCAAGCTTATGCANGCNTGCGGCCGCAATTCGAGCTCGGCCGACTT
GGCCAATTCGCCCTATAGNGAGTCGTATTACAATTCACTGGCCGTCGTTT
TACAACGTCGNGACTGGGAAAACCCTGGCGTTACCCAACTTAATCGCCTT
GCAGCACATCCCCCTTTCGCCAGCTGGCGTAATANCGAANAGGCCCGCAC
CGATCGCCCTTCCCAACAGTTGCGCAGCCTGAATGGCGAANGGAAATTGT
AAGCGTTAATATTTTGTTAAAATTCGCGTTAAATTTTTGTTAAATCAGCT
CATTTTTTAACCAATAGGCCGAAATCGGCAAAATCCCTTATAAATCAAAA
GAATAGACCGAGATAGGGTTGAGNGTTGTTCCAGTTTGGAACAANAGTCC
ACTNTTAAAGAACGNGGACTCCAACGTCAAAGGGCGAAAAACCGTCTATC
AGGGCGATGGCCCACTACGTGAACCATCNCCCTAATCAAGTTTTTTGGGG
TCGAGGNGCCGTAAAGCACTAAATCGGAACCCTAAAGGGAGCCCCCGATT
TAAAGCTTGACGGGGAAAGCCCGGCGAACGTGGCGAAA
SEQ ID NO: 273
CACCTGCAGTCCAAGTACATCGGCACGGGCCACGCCGACACCACCAAGTG
GGAGTGGCTGGTGAACCAACACCGCGACTCGTACTGCTCCTACATGGGCC
ACTTCGACCTTCTCAACTACTTCGCCATTGCGGAGAATGAGAGCAAAGCG
CGAGTCCGCTTCAACTTGATGGAAAAGATGCTTCAGCCTTGTGGACCGCC
AGCCGACAAGCCCGAGGAAAACTGAAACTTTGCTTAACNACCGAATGGNG
GGGANCTTTTCCAACGNTTTT
SEQ ID NO: 274
TTGGTTTCATACTGNTGGGGNTTGAATGNTCCCTNCAACACTNATGTTGA
NACTTAATCCCTAATGNGGCAATACTGAAAGGTGGGGCCTTTGAGATGTG
ATTGGATCGTAAGGCTGTGCCTTCATTCATGGGTTAATGGATTAATGGGT
TATCACAGGAATGGGACTGGTGGCTTTATAAGAAGAGGAAAAGAGAACTG
AGCTTGCATGCCC
nt: 545
SEQ ID NO: 275
GTGGAAGNGACATCGTCTTTAAACCCTGCGTGGCAATCCCTGACGCACCG
CCGTGATGCCCANGGAAGACAGGGCGACCTGGAAGTCCAACTACTTCCTT
AAGATCATCCAACTATTGGATGATTATCCGAAATGTTTCATTGTGGGAGC
AGACAATGTGGGCTCCAAGCAGATGCAGCAGATCCGCATGTCCCTTCNCG
GGAAGGCTGTGGTGCTGATGGGCAAGAACACCATGATGCGCAAGGCCATC
CGAGGGCACCTGGAAAACAACCCAGCTCTGGAGAAACTGCTGCCTCATAT
CCGGGGGAATGTGGGCTTTGTGTTCACCAAGGAGGACCTCACTGANATCA
GGGACATGTTGCTGGCCAATAAGGTGCCAGCTGCTGCCCGTGCTGGTGCC
ATTGCCCCATGTGAAGTCACTGTGCCAGCCCAGAACACTGGTCTCGGGCC
CGATAAGACCTCCTTTTTCCAGGCTTTAGGTATCACCACTAAAATCTCCA
GGGGCACCATTGAAATCCTGAGTGATGTGCACTGATCAAGACTGG
SEQ ID NO: 276
GGAAAGGGCCATTTTATTGCCTAAAACCACCTGGNTTTTNAGGTAACAGT
TCCAACATGTCCTTTTTTGAATAGCTGTTCTAATTATTATATATTCAGCT
GATTAATAGGAGTACTTGATAGGTGGACTGTGTCAGGTAGCCTCAGGCAA
TCCTACTTCAACAAGCTGTCAGGGAGCCATGCCATGCTTCTTTATGACAT
AGGTGAATTTGATAGGCTCACTAGCAGAACATGGGATCACAAGGTGGAAC
CNTTCCNTTT
SEQ ID NO: 277
GACCCCTTCCTTACACCTTATACAAAAAAACTGAAACTGGACCCCTTCCT
TACACCTTATACAAAAATTAACTCAATTTTATTATGTTGTATTAAATTAA
GTTGGGTTTAATTAAGATGGATTAAAGACTTAATTATAAGACCTAAAACC
ATAAAAACCCTAGAAGAAAACCTAGGCCATACCATTCAGGACACGGGTAT
GGGCAAAGACTTCATAACTAAAACACCAAAAGCAATGGCAACGAAGTCCA
AATAGACAAATTGGACCTGATTAAACTAAAGAGCTTCAGCACAGCAGAAG
AGACTATCGTCAGAGTGAACAGGCAACCCACAGAATGGAAGAAAATTCTT
GCAATCTATCCATCTGACAAGGGGCTAATATCCAAAATCTACAAAGAACT
TAAACAAATTTACAAGGAAAAACACAAACAACCCCATCAAAAAGTGGGCT
AAGGATGTGAACAGACACTTCTCAAAAGAAAACATTTATGCAGCCAACAA
ACATGAAAAAAAGTTCATCATCACTGCTCATTAGAGACATGCAAATCAAA
ACCACAATGAGATCCCATCCCACACCAGTTAGAATGGCAATCATTAAAAA
TGT
nt: 268
SEQ ID NO: 278
TTTATGTGTTTTTGCTTGGGGGGCGCTGGGCCTAGCCCAGAGTAGTGCTT
GCTCCCCCTGCCTTGTCCCACCAGGGAGGCAGCAGACTCAGGCCCTCCAT
GGTCCTCTTTGTCATTTTGTTGACATGCATTCCTCCTTTTGTCATCTTGT
TGGGGGGAGGGGATTAACCAAAGGCCACCCTGACTTTGTTTTTGTGGACA
CACAATAAAAGCCCCGTTTATTTGTAAAAAAAAAAAAAAAAAAAAAAAAA
AAAAAAAAAAAAAAAAAA
nt: 569
SEQ ID NO: 279
CTTTAGCCAGCCTGATCAGAAAAAAACAAAAGAAGAGGAAAGACGTAGAT
TACCAACATCAAGAATGTGAGTTATGATATCACTACAGACTCTCCAGGTA
TTAAAAGCATAATTAGAGAATGATATGAGCAGCTATATGCAAATAAGTTC
AACATTGGACAAATGGACAAATTTCTTGAAAGATAAATTATGAAATTTCA
TTCTGAAAGAACTACATGACCTTAATTGTCTTACATCTATTAAATAAGTG
GAAATTGTAGTTTAGAAACTTTCCCACAAAGAAAACTCTAGGCCCAGATG
GCATCAAAATAATATTCAGATGAATGAAATGGAGAAAGGATAGCCTTTTC
AACAAATGGTGGTGGAACAATTGGATTTCCATATGCAAAAAAATAGAGAT
GGACGCAGAGGTGTGTGCTTAGGAGGCTGAGGTGAGAGGATTGTTTGAGG
CCAGCCTGGGCAACATAGCAAGACCCCATTTCAAAAACAAAAATAAAGAA
CTTGTAGCCTTACCTTGTGCCATATTATGAAAATGTATCATAGGCTTAAA
TGTGAAACGTAAAACAAAA
SEQ ID NO: 280
CGCAGGGGCTTCTGCTGAGGGGGCAGGCGGAGCTTGAGGAAACCGCAGAT
AAGTTTTTTTCTCTTTGAAAGATAGAGATTAATACAACTACTTAAAAAAT
ATAGTCAATAGGTTACTAAGATATTGCTTAGCGTTAAGTTTTTAACGTAA
TTTTAATAGCTTAAGATTTTAAGAGAAAATATGAAGACTTAGAAGAGTAG
CATGAGGAAGGAAAAGATAAAAGGTTTCTAAAACATGACGGAGGTTGAGA
TGAAGCTTCTTCATGGAGTAAAAAATGTATTTAAAAGAAAATTGAGAGAA
AGGACTACAGAGCCCCGAATTAATACTAATAGAAGGGCAATGCTTTTAGA
TTAAAATGAAGGTGACTTAAACAGCTTAAAGTTTAGTTTAAAAGTTGTAG
GTGATTAAAATAATTTGAAGGCGATCTTTTAAAAAGAGATTAAACCGAAG
GTGATTAAAAGACCTTGAAATCCATGACGCAGGGAGAATTGC
SEQ ID NO: 281
CGAAAAGCAAATATAACTTGCCACTAACCAAGATCACCTCTGCAAAAAGA
AATGAAAACAACTTTTGGCAGGATTCTGTTTCATCTGACAGAATTCAGAA
GCAGGAAAAAAAGCCTTTTAAAAATACCGAGAACATTAAAAATTCGCATT
TGAAGAAATCAGCATTTCTAACTGAAGTGAGCCAAAAGGAAAATTATGCT
GGGGCAAAGTTTAGTGATCCACCTTCTCCTAGTGTTCTTCCAAAGCCTCC
TAGTCACTGGATGGGAAGCACTGTTGAAAATTCCAACCAAAACAGGGAGC
TGATGGCAGTACACTTAAAAACGCTCCTCAAAGTTCAAACTTAGATTTCA
GATTT
SEQ ID NO: 282
CCGGTCTCTACACAATATATAGAAATCTGGGCATGGTGGTGCCTGGCTGT
AGTCTCAGCTACCTAGTTGGGTGAGGTGGGAGAGTCGCTTGAGTCCTGGA
GGTTGAGGCTGTAGTGAGCCAGGGCTGCACCACTGCATTCCAGCCTGGGT
AACAGAGTGAGACCCTGTCTCAAAAAGAAAAAAAAAAATTGCTAATTTTA
ACAAATCACAAAACTGACTCAGGCAAGTTGTCTGACTCAAAAGCCCTTGA
AAAACCATCAAAGACAGTAGAATGTTAACTGGTCATTTACGTAAAATAGT
GTTCATTAAATTTTTGGTTCATTTAGGATAATCATTTTAAATGAGACTGT
ATTTGAGACTGTATACACATACATATACATGTTTACACACATATACGTAC
AATATATGTACATTCTATCTAAAAGATCATACATGTGTGTACATATATGT
TTTTAAAAGTCAAACTGACATATTAATGGAAACAGTGCTTACATCTCTGG
TAGTGATTTTCTATTAGCAGCAGCCCTACATATGCTGCGTCTCTGAACAG
CATGTCAGTGCCATGACTGTCTAAACATGCAAATATGACTGACAGACTCT
TGAGACAGCTTTCACCTTG
SEQ ID NO: 283
AATTCGNGGCCGCGTCNNCCTANGAGGCACCAGGAAATCCCGCGGGGTGG
CCCATGCAGACCAGGCGCACGTGGCTCATGGGGCANAATTGCCAAGGACA
GCTCACGACAGTGCCACCTTCTCACCATTCCAGCCAAGGAGAGATGTGAC
GTTGGAACTGCTCTGGCACTTCTGTCAAGCCTCCCCCGCCCCAATTGCCT
TGAGATCTCTGCTCTTTGTCAGAGATTTGCAAAGACTCACGTTTTTGTTG
TTTTCTCATCATTCCATTGTGATACTAAGAAACTAAGAAGCTTAATGAAA
AGAAATAAAATGCCTATGTTGTTGTTCT
SEQ ID NO: 284
CTAGAACCCATGACTCCTAGGTCTTATACTGCAACCACAGTATCAGCAAA
TAATCTTTCATAAGGGGATTATTCTCTGATTAACAGGAAATACAGGAATT
TAATTTGTGAACACGCTAGGTAGAAGCAGAAACCCAAATCCAAATCCAAA
TTTAAACATTTAAAATTCATTCTATAACTAAGATCTAACAGTCATTTTCT
TCCCAGTAAGAAATAACCAAAGCATGCTAAAAATCACTGGACTAAATTGG
TGTCAAAACTGCCACATTGCCAGGCATGGGGGGGTCATACTTGTAATCCC
AGCACTTTGGGAGGCCGAGGTGGGAAAATTGCTTGAGGCCAGGAGTTCGA
AACCAGCCTGGGCAACACAGTGAGACCCCATCTCCACAAAAAAAAAAAAT
TAAAAAACAAAACAAAACATTAGCTGGGCATGGTGGTACACGCCTGTAGT
CCCAGCTACTCAGGAGCCTGAAGTGAGAGGATCACTGAAGCCCAGGAGGT
AGAGCTATGACTGTAGTGAGCTATGACTGTGCCACTACACTCCACCTGGG
TGACAGGGGACTC
SEQ ID NO: 285
CGACTTCCATTTGTATTAATGGAATACTAAGTCCCTCTGTGATTTCTGAA
CCAAGCTATTCCTAGGCCTGAGTTTTATTTTGTTGACACAGAAATAAATT
ANAAGGCCAAGCGTGGTGGCATGTGCCTGTAGTCCTAGTTGCTGAGGTAA
GAGGATTGCTTGAGCCCAGGAGTTCAAGGCTGCAGCAAGCTTTGATTGCG
CCACTGCACTCCAGCCTTGGCGACAGACTAAGACGCTGTCTCAAAAAAAA
ACAAAAA
SEQ ID NO: 286
GGTTATCAATGAGATTAAGAGACAACTAGAGTAAAAACAAAAGAAAAGAA
AAGAAANGAAAACAACAGAAGCTCTATTAACTGACCTCTAACCAATACAA
CAGGTTAACTGATGTTCTCCATTCTGTATATAAAAATCCCAGTGGACACC
CACAACACAGGCTTCAGGCTTGTAGGACACTTTCTAGTTCATCTGAGCAC
TTTTGTTCTCAGCAGTTGAGCTGTATACTTAGCAACATTTGGTGCTTCCA
AACCCATTTGTGCCTGTAGCACTTACTATTGAAATACATAATTTAATTAA
ATATTATATAAAGGAATGGAATACGAGTTGGACAAGAAAAAGAGTTAAAT
CTGAAGGTTAGGTAAAAAGAGCAACTTCTTTTCTCTGTTTTGCAGGTTGG
CAAAATCATTTAAAAACAATTGGAAGTATTATATGTTCTGCATTAAGTTG
TCATTTTACTTAAAAACTAGGCATCAAAGATGATGCATAATAAATTTAGT
GTATGCAAGAATGACTGCTTGGGACCTCAATATATGAATTCTTAATCCAA
GGAAAGTCCTTGGCCTTACATTTAAAAGTCGGCAAATAAGTGTACGTTCA
TT
SEQ ID NO: 287
GAACATTTAAAAATAATGCAAATAAGGCTGGGCGTGGGGGCTCACACCTG
TAATCCCAGCACTTTGGGAGGCCGAGGCAGGCAGATCACGAGGTCAGGAG
ATTGAGACCATCCTGGCTAACACAGTGAAACCCTGTCTCTACTTAAAAAA
TAAAAAAATTAGCCAGGCGTGGTGGTGGGCGCCTGTAGTCCCAGCTACTC
AGGAGGCTGAGGCAGGAGAATGGTGTGAACCCGGGAGGCGGAGCTTGCAN
TGAGCTGAGATCGTGCCACTGCACTCCAGCCTGAGCGACAGAGCGAGACT
CTGTCT
SEQ ID NO: 288
TCATTAGAATCCAAGCTTTGAAAATTTCTGATTAATGCTCATGTATTTCT
TTATCTTTGTTTTTCCTTGTGAAGAAAGACTTTCACCACTGTCTGAGTGA
TGATGCTGTTGATAAGGATGATGTCGATGACTACTATATTGCATCTCTCA
GGAACAGCTGATGGGAAGGGAGGGGCTGCTGAGTTCCCTTGTTCTAGCTA
GCAGCACGCTCCTCANAGAGGGGGCCGAGTTACAGACAGCAGCCGCATTC
TCATGCAAAATTAGTTTTAAACTGCTAGTGTGGGCATCGGTACCTTTTGC
CTGGGTGATACCGAAGAATTGTTGAGGATTTAGTATGCTCCGTAGAGACA
GTTCAGCCAGTCATTTCTGCATTGGAGAGACTTCTCATACTTTCTTTGAA
GACTCATAGAAAGCTGGAT
SEQ ID NO: 289
ATTAAGGTTTGTNCCCAACAAGAATAGATGTAATTAGAAAAAANTGNCTT
CCTTACCTATTGCCTCTGATNTTTACTTGCTTAAATTTTTTTTATTGNAA
ATCCAGAAAAAGNGGATTTAGAGAACAACACTAACTCCCACCTAATCTAT
GACAGANATGTACAANANAGTACCTGTGAAAAATGTGAAAGNATNTGAAA
AATGTAACCTTTGGCAGCCTGAGCATAGTCAACCAGAAAAACTATCTGAA
TTAAAATAATTGGTCCATAGGTACTATTTTATTTGGTCCATAAGGATTAT
TTTTTCAACTTTTTTTTCAAGTGTATTATTATGTCATTTCCCACGTAGGT
TACTGATACCTGAAGACTTTTTNCACCTTTAACCTTNCTCGTTGAGGAGC
TTTGTANTCTAATAAAAGAGAAATATAAGTAAATGTTAGATATATGGGNG
GATAATGGTAACTATGTGCTTAAAGAGGTATAAAAGAAGGGTAGGGAGCA
GATAAGACAAAGGAAGGGCTATATTATAANGAAGAATATTCCAAGTAGGG
AAGAGAAAAAGATATGTTATCCATATAATATTTTATGTGCAGTAGAGAAC
ATGTTCTATAGAANAGACAGAAGATG
SEQ ID NO: 290
CTTGAGCCCAGGAATTCCAGCCTGGGCAATATAGTAAGACTCCGTCTCTA
CAAAAGATACAAAAATTAGCCAGATGTGGTGGTGCGTGCCTGTAGTTCCA
GATACTGGAAAGACTGAGGCAGGAGGATTGCTTGAGCATGGGAAGTTGAG
GCTGCAATGAGCTGTGATTACGCCACTACACTCCAGCCTGGGCAACAGAG
TAAGATCTTGTCTCAAAAAAAAAATTGAATTCAGCTAAAAATAATAAAAT
TTTAAAATAATTTTAAAAAGCCCTCAACAGCTTTGTTTTTCTCTCCTTGC
CAGCTTCTCTGCAGCCTATAGCCTGCAGGCTGGCTGCTGCGAGCCAGGAC
AAGCGGTGGGAAATGCAATCACAGCGTGAAATCTCTGTGTTCAGAGACAC
GCAGGAAGCAGGTGAACCATGAAGGGCCAACACATGCCCCCAGTTAGCAG
GGTGTAGAGACCGGGGCAGGGCTTTCTTCTTCCTTCTGGGTTATAAATAT
CCATGTCCTGCCATTTGAAGCTGCAAGTGGCACACATGGATGCTGGACAG
GCGCTCGCACTTTCTGGGCAGGGCANGGGGCTCAAAGGCAGGACAGCTGG
GCAAAAGCACCTTGCGTGGGCCC
nt: 579
SEQ ID NO: 291
CTTTGGAGCTTCTGTCTGTGCTGTGGACCTCAATGCAGATGGCTTCTCAG
ATCTGCTCGTGGGAGCACCCATGCAGAGCACCATCAGAGAGGAAGGAAGA
GTGTTTGTGTACATCAACTCTGGCTCGGGAGCAGTAATGAATGCAATGGA
AACAAACCTCGTTGGAAGTGACAAATATGCTGCAAGATTTGGGGAATCTA
TAGTTAATCTTGGCGACATTGACAATGATGGCTTTGAAGGTAATTAAAAT
TATCAAATTGGTGCTTGATTTCTGCTTTTAAAATGGTTTATGGAAGAAAA
TATGATTAAAGTTTTGTATTGTTTTCCTTCCTATAGAAGATGGAGCCAGA
ATGGCATGCTAAGTTTTTTCTTTTCTTTAGTGTTATATATGACTTCTCCT
CAATTGTCACCCATTGATCTTTACCACTGTTAATAATGGATGATATTCAA
AATACCTTATTTCAGTGATTCTAAGGCACCATTGATTAGAAACTGCATTA
TTATTTATGTGTCCCTAAAAGCTACCTATTAAGCTGTTACACCCACCATT
TTTCTGTTAAGAAAATCCTGATTTCAGAA
SEQ ID NO: 292
GTNNTCCTCTCGGAACGCGCCTTNTGTAGCCAGGTGCTACCAGACCNAAT
ACACGGTTGTTCCAGCTTGCGCATTCACCGATGGCGTAGATATCCGGATC
GGAAGTCTGGCAGGAATCATTAATGACAATACCCCCACGCGGAGCAACGT
CCAGACCACACTGGGTTGCCAGCTTATCGCGCGGACGGATACCGGTAGAG
AAGACGATAAAGTCGACTTCCAGTTCGCTGCCGTCGGCAAAACGCATGGT
TTTACGCGCTTCAACACCTTCCTGCACAATCTCAAGGGTGTTTTTGCTGG
TGTGAACGCGCACGCCCATACTTTCGATTTTGCGACGCAGCTGCTCGCCG
CCCATCTGATCAAGCTGTTCTGCCATCAGCATAGGGGCAAATTCGATAAC
GTGGGTTTCAATACCTAAGTTTTTCAGCGCGCCTGCGGCTTCCAGACCTA
ACAGGCCGCAATTCGAGCTCGGCCGACTTGGCCAATTCGCCCTATAGTGA
GTCGTATTACAATTCACTGGCCGTCGTTTTACAACGTCGTGACTGGGAAA
ACCCTGGCGTTACCCAACTTAATCGCCTTGCAGCACATCCCCCTTTCGCC
AGCTGGCGTAATAGCGAAAGAGGCCCGCACCCGATCGCCCTTTCCAACAG
TTGCGCACCTGAATGGCGAATGGAAATTGTAAGCGTTAATATTTTGTTAA
AATTCGCGT
SEQ ID NO: 293
CTGCGCAGACCAGACTTCGCTCGTACTCGTGCGCCTCGCTTCGCTTTTCC
TCCGCAACCATGTCTGACAAACCCGATATGGCTGAGATCGAGAAATTCGA
TAAGTCGAAACTGAAGAAGACAGAGACGCAAGAGAAAAATCCACTGCCTT
CCAAAGAAACGATTGAACAGGAGAAGCAAGCAGGCGAATCGTAATGAGGC
GTGCGCCGCCAATATGCACTGTACATTCCACAAGCATTGCCTTCTTATTT
TACTTCTTTTAGCTGTTTAACTTTGTAAGATGCAAAGAGGTTGGATCAAG
TTTAAATGACTGTGCTGCCCCTTTCACATCAAAGAACTACTGACAACGAA
GGCCGCGCCTGCCTTTCCCATCTGTCTATCTATCTGGCTGGCAGGGAAGG
AAAGAACTTGCATGTTGGTGAAGGAAGAAGTGGGGTGGAAGAAGTGGGGG
TGGGACGACAGTGAAATCTAA
SEQ ID NO: 294
CTTGTATTCAAGAACTACTGTAATGCATTAGTGGTCTGGCTTCATTTTGT
ATGATGCCAGATCCTTAATTTACCCAGCACAATCATTTCAGTAGTTTCCT
ATGGCTCCTGCAAAAATGCAAACAGAAACCACCACAGGAACAGCCCCTTG
CTGCCTCCTGTTGCTGAGGTAGTAGTCGCTAAAGAAAATTGAAGGCTCCT
TACAATCTATATTTGAAAACTAGAACTTCTGTAGAAACACACAGATCCCG
ATCTTAGAAGTTGTACAGGACAATCTGGTAAAACTGACATAATTGTGATT
TATTAACATGAATTAAAATGCCCAACCAGTGCTTCAGTGTGACAGTATAT
TTAAAATAAAAAAGAAATTAAAGGTCATATACTGTACTACTTTCACAAAG
ATCCACAGTTTTGCAAAAGACTTGTCATATGTACAATGCTATATATCAAA
TGAGAAAAGCTGTAAGCAATTATATACGCAAAAGAAATGGCAGTA
SEQ ID NO: 295
TTCCAGTCCTTTCATTTAGTATAAAAGAAATACTGAACAAGCCAGTGGGA
TGGAATTGAAAGAACTAATCATGAGGACTCTGTCCTGACACAGGTCCTCA
AAGCTAGCAGAGATACGCAGACATTGTGGCATCTGGGTAGAAGAATACTG
TATTGTGTGTGCAGTGCACAGTGTGTGGTGTGTGCACACTCATTCCTTCT
GCTCTTGGGCACAGGCAGTGGGTGTAGAGGTAACCAGTAGCTTTGAGAAG
CTACATGTAGCTCACCAGTGGTTTTCTCTAAGGAATCACAAAGGTAAACT
ACCCAACCACATGCCACGTAATATTTCAGCCATTCAGAGGAAACTGTTTT
CTCTTTATTTGCTTATATGTTAATATGGTTTTTAAATTGGTAACTTTTAT
ATAGTATGGTAACAGTATGTTAATACACACATACATATGCACACATGCTT
TGGGTCCTTCCATAATACTTTTATATTTGTAAATCAATGTTTTTGGAGCA
ATCCCAAGTTTAAGGGAAATATTTTTGTAAA
nt: 496
SEQ ID NO: 296
CAACCCTCTCTCCTCAGCGCTTCTTCTTTCTTGGTTTGATCCTGACTGCT
GTCATGGCGTGCCCTCTGGAGAAGGCCCTGGATGTGATGGTGTCCACCTT
CCACAAGTACTCGGGCAAAGAGGGTGACAAGTTCAAGCTCAACAAGTCAG
AACTAAAGGAGCTGCTGACCCGGGAGCTGCCCAGCTTCTTGGGGAAAAGG
ACAGATGAAGCTGCTTTCCAGAAGCTGATGAGCAACTTGGACAGCAACAG
GGACAACGAGGTGGACTTCCAAGAGTACTGTGTCTTCCTGTCCTGCATCG
CCATGATGTGTAACGAATTCTTTGAAGGCTTCCCAGATAAGCAGCCCAGG
AAGAAATGAAAACTCCTCTGATGTGGTTGGGGGGTCTGCCAGCTGGGGCC
CTCCCTGTCGCCAGTGGGCACTTTTTTTTTTCCACCCTGGCTCCTTCAAC
ACGTGCTTGATGCTGAGCAAAGTTCAATAAAGATTTTGGGAAGTTT
nt: 397
SEQ ID NO: 297
CGGATGTGGTGGCAGGCGCCTCTAGTCCCAGCTACTCGGCAGGCTGAGGT
AGGAGAATGGCTTGAACCCAGGAGGTGGAGCTGACAGTGAGCCGAGATCG
CGCCACTGCACTCCAGCCTGGGCGGCAGAGCGAGACTCCATCTCAAAAAA
AAAAAAAAAAAAAATAGACTTTGAGACCAGCCTGACCAACATAGTGAAAC
CCGTCACTACTAAAAATACAAAAATTACCCGGGCGTGGTGACGGGCGCCT
GTAATCCCAGCTACTTGGGAGGCTGAGACAGGAGAATCACTTGAACCAGG
GAGGCGGAGGTTGTAGTGAACTGAAATCGTGCCCCTGCACTCCAGCCTGG
GTAACAAGAGCGAAACTCCGTCTCAAAAATAAATAAATAAATAAAAT
nt: 293
SEQ ID NO: 298
CCAGCTTTTTATGGTGTTTAATCTAATACACTTAAGCTGCAGTCCCAAAA
TTAGGGGTCCTTCAGTCTTGGAGACTATAAGGGAGCCTCTGCACCCAGGG
AAAATGTTACCCTTTACAGGGGGGAAGGGTAAACCAGTAGGGAATACAGT
ACAATCCCAACCCTACTGGGAGGGGCGGGAGGGAGGTGTTGCCGTCACTG
TATTAAGTCGATGTTGGGAAACGTTTTAACATCTGGAGCCTTTGTGGGTG
GAAATATGTCTCCAGTTACAACTCCGCAGTGGATGTGAAGAAG
SEQ ID NO: 299
GGAAGCTACAATGATTTTGGGAATTACAACAATCAGTCTTCAAATTTTGG
ACCCATGAAGGGAGGAAATTTTGGAGGCAGAAGCTCTGGCCCCTATGGCG
GTGGAGGCCAATACTTTGCAAAACCACGAAACCAAGGTGGCTATGGCGGT
TCCAGCAGCAGCAGTAGCTATGGCAGTGGCAGAAGATTTTAATTAGGAAA
CAAAGCTTANCAGGAGAGGAGAGCCAGAGAAGTGACAGGGAAGCTACAGG
TTACAACAGATTTGTGAACTCAGCCAAGCACAGTGGTGGCAGGGCCTAGC
TGCTACAAAGAAGACATGTTTTAGACAAATACTCATGTGTATGGGCAAAA
AACTCGAGGACTGTATTTGTGACTAATTGTATAACAGGTTATTTTAGTTT
CTGTTCTGTGGAAAGTGTAAAGCATTCCAACAAAGGGGTTTTAATGTANA
TT
SEQ ID NO: 300
TGGATTCCCGTCGTAACTTAAAGGGAAACTTTCACAATGTCCGGAGCCCT
TGATGTCCTGCAAATGAAGGAGGAGGATGTCCTTAAGTTCCTTGCAGCAG
GAACCCACTTAGGTGGCACCAATCTTGACTTCCAGATGGAACAGTACATC
TATAAAAGGAAAAGTGATGGCATCTATATCATAAATCTCAAGAGGACCTG
GGAGAAGCTTCTGCTGGCAGCTCGTGCAATTGTTGCCATTGAAAACCCTG
CTGATGTCAGTGTTATATCCTCCAGGAATACTGGCCAGAGGGCTGTGCTG
AAGTTTGCTGCTGCCACTGGAGCCACTCCAATTGCTGGCCGCTTCACTCC
TGGAACCTTCACTAACCAGATCCAGGCAGCCTTCCGGGAGCCACGGCTTC
TTGTGGTTACTGACCCCAGGGCTGACCACCAGCCTCTCACGGAGGCATCT
TATGTTAACCTACCTACCATTGCCCTGTGT
nt: 498
SEQ ID NO: 301
GTGGTACATATACACAAAGGAAAACTATGTAGCCATTAAAAGAAAAGGAA
CTCCTATCATTTGTAACAACATAAATAAATCTGGAGGAGATTAGGCTAAG
GTGAAATAAGCCAGGCACAAAAAGACAACTACCATATGATCTTACTTATA
CGTGTGTGGAATCTAAAAAGGTGGAATTTACAGAAGCAGAGAGTAGAATG
GTGATTACCAGAGGCTGGGGAGTGAGGGCAGGAGGTTGGAGAAATGTTGG
TCAAAGGATACAAAGTTTCAGTTATACAGGATGAATAAGTTCAAGAGATC
TATTGTACAACGTGGTGGCTATAGTTGATAACAATGTATTGTGTTCTTGA
AAAATGCTGAGAGAGTAGATTTTAAGTGTTCTCACCACAAAACATAAGTA
TGTGAGGTAATGCATGTGTTAATTANCTTAATTTAGACATTTCATAATGT
ATTATACATATTTCAAAACCACGTTGTACATGAGAAAGATACACAATT
SEQ ID NO: 302
GCCCAGTCGACCCATGTTCTCCTTTCTACACCAGCATTAGACGCTGTCTT
CACAGATTTGGAAATCCTGGCTGCCATTTTTGCAGCTGCCATCCATGACG
TTGATCATCCTGGAGTCTCCAATCAGTTTCTCATCAACACAAATTCAGAA
CTTGCTTTGATGTATAATGATGAATCTGTGTTGGAAAATCATCACCTTGC
TGTGGGTTTCAAACTGCTGCAAGAAGAACACTGTGACATCTTCATGAATC
TCACCAAGAAGCAGCGTCAGACACTCAGGAAGATGGTTATTGACATGGTG
TTAGCAACTGATATGTCTAAACATATGAGCCTGCTGGCAGACCTGAAGAC
AATGGTAGAAACGAAGAAAGTTACAAGTTCAGGCGTTCTTCTCCTAGACA
ACTATACCCGATCGCATTCAGGTCCTTCGCAACATGGTCACTGTGCAGAC
CTGAGCAACCCCACCAAGTCCTTG
SEQ ID NO: 303
CTGTAACAGAGATTCCTTTTTTCAATAATCTTAATTCAAAAGCATTATTA
GACTTGAAAGGGTTTGATAATCTCCCAGTCCTTAGTAAAGATTGAGAGAG
GCTGGAGCAGTTTTCAGTTTTAAATGAGTCTGCAGTTAATATCAAATGTG
AGTTTGGGACTGCCTGGCAACATTTATATTTCTTATTCAGAACCCTTGAT
GAGACTATTTTTAAACATACTAGTCTGCTGATAGAAAGCACTATACATCC
TATTGTTTCTTTCTTTCCAAAATCAGCCTTCTGTCTGTAACAAAAATGTA
CTTTATAGAGATGGAGGAAAAGGTCTAATACTACATAGCCTTAAGTGTTT
CTGTCATTGTTCAAGTGTATTTTCTGTAACAGAAACATATTTGGAATGTT
TTTCTTTTCCCCTTATAAATTGTAATTCCTGAAATACTGCTGCTTTAAAA
AGTCCCACTGTCAGATTATATTATCTAACAATTGAATATTGNAAATATAC
TTGGCTTACCTCTCAATAAAAGGGTCTTTTCTATT
SEQ ID NO: 304
TCCACCCACCTTGACCTCCCAAAGTGCTGGGATTATAGGCGTGAGCCACC
TCGCCCAGCCCGATACTAGGACTTATGCAGAAAAAACCTTGACATGGAGG
AAAGTAAGATCTAAATAAATACTGTATTCATAGATTAAAAGACTCAGCAT
AATAAATATACCATTTCTCCCCAGATTGATGTACAGATTTAACACAATTC
CTATCAAGATCCCAGCAAGATTTTTGTAGATATGTAAAAGATTATTCAAA
AATGTAAAAGGAAGGACAAAGGACTAGAATAGATAAAACAAAATGGAGAA
AGATTTAATAGGAATCACTGTAACTGATTTTAAGACATACAGAACAATAA
TAGAAACTGCTTGTATTAGTCCATTTTCACGCTGCTGATAAAGACATACC
TGAGATTGGCAATTACAAAGGAAAGANGTTTATTGGCTTACAGTTCCCAT
GGCTGGGGAGGCCT
SEQ ID NO: 305
CTCCTCTGGGTTGAAACCCGGGCGCCGCCAAGATGCCGGCTTACCACTCT
TCTCTCATGGATCCTGATACCAAACTCATCGGAAACATGGCACTGTTGCC
TATCAGAAGTCAATTCAAAGGACCTGCCCCCAGAGAGACAAAAGATACAG
ATATTGTGGATGAAGCCATCTATTACTTCAAGGCCAATGTCTTCTTCAAA
AACTATGAAATTAAGAATGAAGCTGATAGGACCTTGATATATATAACTCT
CTACATTTCTGAATGTCTGAAGAAACTGCAAAAGTGCAATTCCAAAAGCC
AAGGTGAGAAAGAAATGTATACGCTGGGAATCACTAATTTTCCCATTCCT
GGAGAGCCTGGTTTTCCACTTAACGCAATTTATGCCAAACCTGCAAACAA
ACAGGAAGATGAAGTGATGAGAGCCTATTTACAACAGCTAAGGCAAGAGA
CTGGACTGAGACTTTGTGAGAAAAGTTTTCGACCCTCAGAATGATAAACC
CAGCAAGTGGNGGGCTTGCTTTGTGAAGAGACAGTTCATGAACAANAGTC
TTTCAGGACCTGGACAGTGAAGGGAGCCCGGGCAGCCA
SEQ ID NO: 306
CGNGGCCGCGTNAACTTTTGATCGTCAGCTGGGGCTGGCAGGCACCTAAA
TGGGAAGGGTGATAGCAGTGTGTTGGGGGGAGTTTAGGGAACGGTCCTCT
ACCGATAGAGGCAGCANCTCATTGGAATTTCCTCCTGAAGTTGTCTTGCC
CCTTGAATCCTGCAGGAAGGCTGGCAAATGGCCATTTCCCTTCCACTTGA
ATAGAGACCCATAACTCAAGTATCTGCCCTTAAGACACCACAGGACTGTT
CTTCGCGGGCCCTGCCCCTGGATTTGGGAGAGGCAGTCCANCTCACCCAA
CTAGGCTCTGCANGGGGACCANGAGGGATGGGTTGTGTCCACAGGACCAG
CCAGACTGATGAGGGATGCGGCAAGCATATTCTCACCACCTTCTTTCACG
TTTACAACANACCAGCNTTCCCTGTGTGGCAGGGGTTACATTGGTCACCG
AGGACCTANAATCATGGAGTGCTCTGGGGATCCGGGCTTGGA
SEQ ID NO: 307
TCAGTGTTGAATTTTGTCAGACACTTTCTCTGCATCAATTGGTATGACCA
TGTGATTTTTTTTCTGTAGCCTGTTAATATGGTTAATTTTCAAATATTGA
GCTGATTAATTTTCAAATATTGAGCTCTCCTTGCATCTCTGGAATAAGTA
CCACTTGGTCGTGGTATATATTTCTTTTAATATATTGCTGAATTCTGTTT
GATCATGTTTTCTTAAAGACTTTCGTGTCTGTTTTCATGATAGATACTGG
TCTATAGTTTTGTTGTAATATCTTGGTTTGATTTTGATATCAGGATAATG
CTACCTTAATAGAATGAATTGGAGCCAAGTATGGTGGCAAATGCCTATAG
TCCTAGCTACTCAGGAGGCTGAGGTGGTGGGGACTGCTTGACCCANGAGT
TCAAATCTAGCTTGGGCAATGTAGCAAGAC
SEQ ID NO: 308
TAGAAGGAATGACTATTCATGTCCAAAGTGAATGGTTTTGTGCAGTGAAC
AACACATGGCGAGGTACTAACTGAGAAACTTTTTCATGCTTTATGCCTAC
CTCTTGTAGTTGTTGCAGAGCAAATATAAATTGTAATAAGATAGCTAGGC
CTTGCAGAAACAAACAGAAAAACTTAAAAAAAAATGATATAAGAGCTGGA
GTCTAGTATTTATATGAATCTGTGAGAGATAATTTTTTTGGTCTCACTGC
AATGAACCAAAAGCGGCTGAGTTTGGTTTTTAATTGTAGCCATGTATTGA
AGGCATCTTTTTGACCAACTCTTGTTGGTTCTGTCTTGAACCATTGTTAA
TCACTGTGCTGTAATTAGTATAGCTAAATCTTTTCCTTCCTTGCTCCTCC
CCCAGCCCACCCCGTCTTCCCTTAACATTTTTTCAGGGGGGGTTGGGAGT
GGTTTCATTTTAATGTGAGTGGATGTTTTGATAGTTGTAAGGAAAAAATG
CATTTCAGACACATTTCACACATGAGCTATTTTCTTACACAGTATGTCTT
ATTGGTAATAAGAATGTAATTCAT
SEQ ID NO: 309
CNTTCCNTAAGAATACAAAAAATTAGCTGGGCGTGGTGGCAGGCGCCTGT
AATCCCATCTACTCAGGAAGCTGAGGCTGGAGAATCGCTTGAACCCGGGA
GGCGGAGGTTGCAGTGAGCAGAGATCACGCCACTGCAGTCCAGCCTGGGC
AACAGTGCGAGACTCTGTCTCAAAAAAAAAATAAATAAATTACCTGGGTG
TGGCAGCGCGTGCCTGTAATCCCAGCTACCCAGGAGGCTGAGGCAAGAGA
ACTGCTTGAACCCAGGAGGCAGAGGTTGCATGGAGCTGAGATGGCGCCAC
TGCACTCCAGTCTGGTGACAGAGTGAG
SEQ ID NO: 310
CTCTCTACTAAAAATACAAAAATTAGCTGGGCACGGNGGTGCATGCCTGT
AAACCCAGCTACCAGGTACTCGGGAGGCTGAGGCAGGAGAATCGCTTGAA
CCAGGGAGTCGGAGGTTGCGGCGAGCTGAGATCATGCCACTGCACTGCGG
CCTGGAGACAAGAGCAAGACTCCGTCTCAAAAAAAAAAAAAAAAAAAAAA
AAAAAAGACNTCACCTAATTGCAGNGNGNGGACCTTATTTGGCTNTTAAT
TCAAACTATTAAAAATGTGAACN
nt: 260
SEQ ID NO: 311
CGGGGTCTGTACCGGGCTGGCCTGTGCCTATCACCTCTTATGCACACCTC
CCACCCCCTGTATTCCCACCCCTGGACTGGTGGCCCCTGCCTTGGGGAAG
GTCTCCCCATGTGCCTGCACCAGGAGACAGACAGAGAAGGCAGCAGGCGG
CCTTTGTTGCTCAGCAAGGGGCTCTGCCCTCCCTCCTTCCTTCTTGCTTC
TCATAGCCCCGGTGTGCGGTGCATACACCCCCACCTCCTGCAATAAAATA
GTAGCATCGG
SEQ ID NO: 312
CTGAGTNTAGAAATGATGCCATTAATACTGATTGCAAAAACATTACAACT
CAGTACTGCAGCTTTCATTCAAATAGGTTATATGTATAAACTGAGTTCAA
CAATATTGTATTTGAGATGGTAAAGTTAAAGAAATGCAATAATGTAAATA
ATACTTAAGAAAATAAGATCTCAGGAAACTGTATATACTCTGTACTTTTA
TGCAACTTTATCAGATCATTTCAGTATATGCATCAAGGATATAGTGTATA
TGACATGAACTTTGAGTGCAAAAACTGTACTATGTACCTTTTGTTTATTT
TGCTGTCAACATCTAAATAAAGGTTTTTTTGTTTGTTTTTTGTTTTTTTA
ATTGTTTTGTTTTAAAGATTGTTTTAATTAATTAAAAAATTAATTGTTTT
AATTAAACAATTGTTTAATTGTTTTAAAGTCGCCAGGCTGAGGCAGGTGA
ATCACAAGCTTAGGAGTTGGAGGCTAGCCTGCCAACATGGTGAAACCCCG
TCTCTACTAAAAATACAAAAAAATTAACTGGGTGTGGG
SEQ ID NO: 313
CCCATCTGCACCAGTACACAGGCAGGCATTATCATTCTTCACCTACTTTT
TAAATAGTGGCAACTTGGGATTCATTCTGGTGATTCTGAACCTTGCCTCA
TAGCTTAAAGTATAAAAAAGATTCAAGAGCAGTGAGGTTTGTTCTTTCCA
GTGAATGGTGGACTGAGTGGTGCGAGGTGGAGGGCTAACAAGAGGAAAGA
ACTACATTCTTCAGAATACAGTGATGAAAATTCATTTTGAAACTCAAATA
TTTTCATTTTGGATATTCTCCTGTTTTTATTAAACCAGTGATTACACCTG
GCCATCCCTCTAAATGTTCTAGGAAGGCATGTCTATTGTGATTTTGATGA
AGACAGAATTATTTTTCTCTGTAGAAACACAGATACCACTTTATCAGGGG
AAGTTAGTCAAATGAAATGGAAATTGGTAAATGGACAAAAGCTAGCTAGT
AAAAAGGACGACCCAGCAACATGCTTTAACCCCATTGTATGTTTGTGGAA
AGAGCATAGTTTAACATCTTGAGAAATTTGGGACATAAAAGTTTTCATNG
GTAGACAGTTCATGGCAGTATATGAATTGACATAATGGAAATAATCTGAT
TTTATTTTTACAACTAACATCCTTTCCCC
nt: 641
SEQ ID NO: 314
GGAATTCCAAGTGCTTGGGGATAATGATACCTCTGACCTTTCTTCCTTTT
GGGAAGTACTTGAGTGTGCAGCTGCATGAGGCCTCAGCAGGAGAGAGATT
TTAGGTCCAAGAAGCTATACCAGTAGGACAAGGCAGGAAAATACTACACT
TTCAGGATCAAGCCCCTCTGACTCTCATTTGGAAACTGGATGTTTGCTAA
GCACCTGCTTCTTAAGGATGCCGAGGGATTTAATGATACTCCCAGAAACC
TGGAGAGATTAATGGGGCCTATGGAGAAGTGCTCTGAACTCAGTGTTGGG
ACTTGAATAAAATTAACCATTGTCATGTTTTCAGAACAACTAAGCTGTTT
TATATTTCATGTGCATGAAAGCCCTAGAACTAAGTTGTGTTATTTCCAGA
AATGAAATAGATCCCACAGTTAGATGATGTGGCCATTAGGAAGTACCAAA
TTTATAAAAATCACTGGAGGTCTGTCTGAGCAGTACCTAATAAAATATAG
TATACTGAAAGTGAACAGATACTTTGTCTCTTTCTTTGGCTGCTTGATCT
TTATCTGTGTCTGCCGTACAGTGCACCCTTAAAGTATTCTACACCAGTGC
TTCTCAAACTGGAAATGTGCATGTAAGTCACCCANGGGTCT
SEQ ID NO: 315
TGCATGCCCATAGTCCCAGCTATTTGGGAGGCTGAGGCAGGAAAATCGCT
TGAACCCGGGAGCCAGAGGTTGCAGTGAGCCGAGATCGCACTCCAGCTTG
GCGACAGAACAAGACTCTGTCTCAAAAAAAAAAAAAAAAGAAATCTTGGG
ATCCTGAACCCCTTACTCGAAGGGCTAAGGTAGCATCTCAGCATGTCTTA
TTCGAGACTTCGTANAACCAGACCTGCTGTTTGTAGATGTTAATTAATCA
AACCTTTCTCTACTCATTCTGGACCAGTTAAGGTTTTCTCCTTCTCCGTA
TGAGTTTTGATTTTCGTCCTCCTTGGTTGGAGATCACACTTTGGTCTGCT
GCTAAGTTGGATGCCTCCCACTGTCTTTCCCTAAGTCTAGGGCTTCANAC
CCCAGTGTGGGGAGAGGGACTTTCGTTTCCTGCCCCTCACCACATCAGAC
ACAGGCAGGCAAGAATAAGATGGCCAAAAGGCCGATGAACTTCTTGACCT
AGCCTGGGACATTACCTGTTACTAGGTGGACTTCACTGCCTGTGAATGGA
AGCTGAAGGGCTGTTTTTTTGGTTTGTATTTGGACAGGCCAGGCTTANAG
AGGGAGAGAACTGGGCTACTCTTCAGCAGTGATCTTTAAAATGCC
SEQ ID NO: 316
CAGAGTGCAAGACGATGACTTGCAAAATGTCGCAGCTGGAACGCAACATA
GAGACCATCATCAACACCTTCCACCAATACTCTGTGAAGCTGGGGCACCC
AGACACCCTGAACCAGGGGGAATTCAAAGAGCTGGTGCGAAAAGATCTGC
AAAATTTTCTCAAGAAGGAGAATAAGAATGAAAAGGTCATAGAACACATC
ATGGAGGACCTGGACACAAATGCAGACAAGCAGCTGAGCTTCGAGGAGTT
CATCATGCTGATGGCGAGGCTAACCTGGGCCTCCCACGAGAAGATGCACG
AGGGTGACGAGGGCCCTGGCCACCACCATAAGCCAGGCCTCGGGGAGGGC
ACCCCCTAAGACCACAGTGGCCAAGATCACAGTGGCCACGGCCACGGCCA
CAGTCATGGTGGCCACGGCCACAGCCACTAATCAGGAGGCCAGGCCACCC
TGCCTCTACCCAACCAGGGCCCCGGGGCCTGTTATGTCAAACTGTCTTGG
CTGTGGGGCTAGGGGCTGGGGCCAAATAAAGTCTCTTTCCTC
nt: 583
SEQ ID NO: 317
GAACCCTGCGGAGGGACTTCAATCACATCAATGTAGAACTCAGCCTTCTT
GGAAAGAAAAAAAAGAGGCTCCGGGTTGACAAATGGTGGGGTAACAGAAA
GGAACTGGCTACCGTTCGGACTATTTGTAGTCATGTACAGAACATGATCA
AGGGTGTTACACTGGGCTTCCGTTACAAGATGAGGTCTGTGTATGCTCAC
TTCCCCATCAACGTTGTTATCCAGGAGAATGGGTCTCTTGTTGAAATCCG
AAATTTCTTGGGTGAAAAATACATCCGCAGGGTTCGGATGAGACCAGGTG
TTGCTTGTTCAGTATCTCAAGCCCAGAAAGATGAATTAATCCTTGAAGGA
AATGACATTGAGCTTGTTTCAAATTCAGCGGCTTTGATTCAGCAAGCCAC
AACAGTTAAAAACAAGGATATCAGGAAATTTTTGGATGGTATCTATGTCT
CTGAAAAAGGAACTGTTCAGCAGGCTGATGAATAAGATCTAAGAGTTACC
TGGCTACAGAAAGAAGATGCCAGATGACACTTAAGACCTACTTGTGATAT
TTAAATGATGCAATAAAAGACCTATTGATTTGG
nt: 424
SEQ ID NO: 318
CTTGGCTCCTGTGGAGGCCTGCTGGGAACGGGACTTCTAAAAGGAACTAT
GTCTGGAAGGCTGTGGTCCAAGGCCATTTTTGCTGGCTATAAGCGGGGTC
TCCGGAACCAAAGGGAGCACACAGCTCTTCTTAAAATTGAAGGTGTTTAC
GCCCGAGATGAAACAGAATTCTATTTGGGCAAGAGATGCGCTTATGTATA
TAAAGCAAAGAACAACACAGTCACTCCTGGCGGCAAACCAAACAAAACCA
GAGTCATCTGGGGAAAAGTAACTCGGGCCCATGGAAACAGTGGCATGGTT
CGTGCCAAATTCCGAAGCAATCTTCCTGCTAAGGCCATTGGACACAGAAT
CCGAGTGATGCTGTACCCCTCAAGGATTTAAACTAACGAAAAATCAATAA
ATAAATGTGGATTTGTGCTCTTGT
nt: 626
SEQ ID NO: 319
GATTTTTTTTTTTTTTTTGAGATGGAGTCTTTCTCTGTCGCCCAGGCTGG
AGTGCAGTGGTGAAATCTCGACTCACTGCAACCTCCGTCTCCTGGGTTCA
AGCAATTCTCCTGCCTCAGCCTCCTGAGTAGCTGGGATTACAGGCACCAG
CCACCACGCCCGGCTAATTTTTGTATTTTTAGTAGAGACAGGTTTTCACC
ATGTTGGCTAGGCTGATTTTGAACTCATGACCCCAAGTGATCTGCCCGCC
TCGGCCTCCCAAAGTGCTGGAATTACAGGTGTGAGCTACCACTCCCAGCC
AATGATTACATTTATAAGGTAAAATAACTTGTGCCAATCTGTACAAGTGA
ATTCAGATTTAAAATTTTAATTGTAAAAAGATATCCAGGTGATATTTCTC
CCTGAATAATTTAGTTTCCTTTTCTATTTCTTGATATAAAAGTACTCAGC
ATTGAAGTAATTGCTATCTTCACATTTCTTCCTATTTGAGCTGTCTAAAT
AAGTAGTCCTACATATTTTCCCCCCAACACAAAAAACCCAGAAAAGAATT
ATTTTATACTGGATTTTTTTGGTTGTAGCAGGAACCTAAAGGNGCCAATT
GTAACATGCATGTTCTTTTTGGCAAA
SEQ ID NO: 320
GTCCATCCTGCAGGCCACAAGCTCTGGATGAGGAACTTGAGGCAAGTCAC
CAGCCCCTGATCATTTCGCCTAAAAGAGCAAGGACTAGAGTTCCTGACCT
CCAGGCCAGTCCCTGATCCCTGACCTAATGTTATCGCGGAATGATGATAT
ATGTATCTACGGGGGCCTGGGGCTGGGCGGGCTCCTGCTTCTGGCAGTGG
TCCTTCTGTCCGCCTGCCTGTGTTGGCTGCATCGAAGAGTAAAGAGGCTG
GAGAGGAGCTGGGCCCAGGGCTCCTCAGAGCAGGAACTCCACTATGCATC
TCTGCAGAGGCTGCCAGTGCCCAGCAGTGAGGGACCTGACCTCAGGGGCA
GAGACAAGAGAGGCACCAAGGAGGATCCAAGAGCTGACTATGCCTGCATT
GCTGAGAACAAACCCACCTGAGCACCCCAGACACCTTCCTCAACCCAGGC
GGGTGGACAGGGTCCCCCTGTGGTCCAGCCAGTAAAAACCATGGTCCCCC
CACTTCTGTGTCTCAGTCCTCTCAGTCATCTCGAGCCTCCGTTCAAAATG
ATCATCATCAAAACTTATGTGGCTTTTTGACCTTTGAATAGGGAATTTTT
TAAAATTTTTTAAAAATT
SEQ ID NO: 321
CCAGCGCAGGGGCTTCTGCTGAGGGGGCAGGCGGAGCTTGAGGAAACCGC
AGATAAGTTTTTTTCTCTTTGAAAGATAGAGATTAATACAACTACTTAAA
AAATATAGTCAATAGGTTACTAAGATATTGCTTAGCGTTAAGTTTTTAAC
GTAATTTTAATAGCTTAAGATTTTAAGAGAAAATATGAAGACTTAGAAGA
GTAGCATGAGGAAGGAAAAGATAAAAGGTTTCTAAAACATGACGGAGGTT
GAGATGAAGCTTCTTCATGGAGTAAAAAATGTATTTAAAAGAAAATTGAG
AGAAAGGACTACAGAGCCCCGAATTAATACCAATAGAAGGGCAATGCTTT
TAGATTAAAATGAAGGTGACTTAAACAGCTTAAAGTTTAGTTTAAAAGTT
GTAGGTGATTAAAATAATTTGAAGGCGATCTTTTAAAAAGAGATTAAACC
GAAGGTGATTAAAAGACCTTGAAATCCATGACGCAGGGAGAATTGCGTCA
TTTAAAGCCTAGTTAACGCATTTACTAAACGCAGACGAAAATGGAAAGAT
TAATTGGGAGTGGTAGGATGAAACAATTTGGAGAAGATAGAAGTTT
SEQ ID NO: 322
GAGGAAAGGGGAGTTAATATTTAGTGGACAGAATTTCAGTTTTACAGATG
AAAAGAGTTCTGGAGATAGACGGTGTTGATAGTTGCACAGCAGTGTGAAT
GTGCTCATTGTTACCGAACTTAAAAATGTTTAACATAGTATTATGTGATT
TTTATTTTGCCACTTAAAAAAAAAGAATGAAGTACTGATACATGCTACAA
CATGGGTGAGCTTTAAATACATTCTGCTCAGTGAAATAAGCCAGATGCAA
AAGATCACATATTATATAATCCACTTATACGAGATACCTAGAATAGGCAA
ATTCATAGAGACAGAAAGTAGAATAGTGGTTCCCAGGGGCTGGGGACAAG
GGGGCAGTGAGAGATTGAGAGTTATTATTAATGCGTACAGAGTTTCAGTT
TGGGCTGATAAAAAAGTTCTGAAGATGGATGGTGATGATGGTTGTACATC
AATGTGAGTGTAATTACCGCCACTGAACTGCCCTTAAAAACGTTTAAAAG
AGTAAATTTTATGTTGNGTATATTTTACCATAAT
SEQ ID NO: 323
TTTTTTTTTCATAAGAGGCAAGTACAAGAAAAAGCTTAATTACTTTAACT
TCTAAGTAGTTTGGAATCTAAATAAATAGGAGTTACCAAATATATGCGCT
TCTGTGAATAGTTTTCCCCCACATGTTTATTTATATTTTTGCATCTCATC
AAACCTAACAGATTCTAAAGTCTCTGGTGATAATGACAATATCTGCTACG
GAGAGACTAGCCTGGGGGAAGAGGATCTCCCTGAACAAGGATAGCGGAGT
TGCTGCAGCTTTCAAATGAAGCTGGACATTTAGCTGCGGGGGTAGCACCC
TTTGATCAAGGCAGCCCAAAGATGAGTTTCAGGGATGGGACTGACAGAAG
AGAAAAGTTCTTCCCAGCCCTTTCTACTTTTTCTCTTTGTTTCTCAGGCT
TCTGGCCGTCTTCAGTTTTCACAAGTTTCACTCTCAACCCTAAACAGTAC
TTCTGTGAAGTACCCTTTGGCCCCTCGTTTTCAGCTCCTAAACTCACCTG
GAAATAGATGTCAATCTAATTTTGGGTCTGACTAGTGCAGTAGGCATTTT
TGGTGA
SEQ ID NO: 324
CTCACACAGAACAAAAATGAATGAGTGTGGCTGTGTGCCACTATCACTGT
GTCTACAAAAACAGCCAGTGGGCCTGATTTGGCCCTTGGCTGCAGTGCGC
CCGTCTCTGTTTTTGAGGAATAAAATCGCATCATTTCATATGGCTAATGC
AATTTTTTTCCCATCTGGAAGCAACATCTGATTGGACTCATCTTGTATGG
TGCTTGTTACAGTCTCTGTAAATGGGAGAGGGTCCGAGAATAGCTCTTCC
TGTTTTCATCAGGACTGTTTTTAGGGATGGCAAAGAAGTCAGTGTGTCCA
GCCTGTGTCCTCCTCACCACGTGGCTGATTCCTGAATCTGCATGTGCANC
ACNTGCCGTTGTCTGGGGCATGATCTGTGTGA
nt: 556
SEQ ID NO: 325
CTTTTCTCTGGGTATAGATTTACCCTAGCACCTATCTCATTATATTGAAT
TTTCCAGCATATTTAAATAAACTATTAATTAGTCACACTATTTCTTAAAA
GTCACACTATCAACTAATCGTGACCGCAATTATCTAGGGGTGATAATCTG
CTGAGTCTACTCTTTAAATACACTGGGACCCAGCATATTGAGTTATATTG
GCACAGAAACTTCACTCTGGGTATAGATTTACCCTAGTACCTTGCCGGCA
GGATCCTATTATTCATGGTTGTACAAGCAAGGTTCAGGGAAGAGGCTGGC
ACAGAGAAGGTACCTGGTAACTGTTGTTTGAGGCTGAATTCAGCTCAACT
CAGCTCCAGTAGAGATGGTGTCCCCTTCTCTACCGTGTTGAGATAGTGTG
CAGTCCCTTCCTAAGGGCTGTTACCCACCGCAATAGGACTTGTCAGCTTC
AACTTTTAAATTTCTCTGCTCCCGCTGGGACCCACCCGCTTCAAAAATCA
TCATGGNGGNTTTAGCACCAATTTAGTAAACACAAACTGTCTGAAATATT
TTGGAT
SEQ ID NO: 326
GAACATTCAAGATAGTGAGAGGAAGAAAAAGATATGGCTGTACGGGACCG
AGGTCTCTTCTATTATCGCCTCCTCTTAGTTGGCATTGATGAAGTTAAGC
GGATTCTGTGTAGCCCTAAATCTGACCCTACTCTTGGACTTTTGGAGGAT
CCGGCAGAAAGACCTGTGAATAGCTGGGCCTCAGACTTCAACACACTGGT
GCCAGTGTATGGCAAAGCCCACTGGGCAACTATCTCTAAATGCCAGGGGG
CAGAGCGTTGTGACCCAGAGCTTCCTAAAACTTCATCCTTTGCCGCATCA
GGACCCTTGATTCCTGAAGAGAACAAGGAGAGGGTACAAGAACTCCCTGA
TTCTGGAGCCCTCATGCTAGTCCCCAATCGCCAGCTTACTGCTGATTATT
TTGAGAAAACTTGGCTTAGCCTTAAAGTTGCTCATCAGCAAGTGTTGCCT
TGGCGGGGAGAATTCCATCCTGACACCCTCCAGATGGCTCTTCAAGTAGT
GAACATCCAGACCATCGCAATGAGTAGGGCTGGGTCTCGGCCATGGAAAG
CATACCTCAGTGCTCANGATGATACTGGCTGTCTGTTCTTAACAGAACTG
CTATTGGAGCCTGGAAACTCAGAATGCAGATCTTTTGTGAACAAAATGAA
GCAAGAACCGGAGACNCTGAATAGTTTTATTTCTGTATTAAAAACTGNGA
TTGGAACAATTGAAGA
SEQ ID NO: 327
CCACTCCACCTTACTACCAGACAACCTTAGCCAAACCATTTACCCAAATA
AAGTATAGGCGATAGAAATTGAAACCTGGCGCAATAGATATAGTACCGCA
AGGGAAAGATGAAAAATTATAACCAAGCATAATATAGCAAGGACTAACCC
CTATACCTTCTGCATAATGAATTAACTAGAAATGAGGATTCTGACCTTGA
CTTTGATATCAGCAAATTGGAACAGCAGAGCAAGGTGCAAAACACAGGAC
ATGGAAAACCAAGAGAAAAGTCCATAATAGACGAGAAATTCTTCCAACTC
TCTGAAATGGAGGCTTATTTAGAAAACAGAGAAAAAGAAGAGGAACGAAA
AGATGATAATGATGATGAGTCAGGTAAAAGTTCCAGAAATGTGAACAACA
AAGATTTTTTTGATCCAGTTGAAAGTGATGAAGACATAGCAAGTGATCAT
GATGATGAGCTGGGTTCAAACAAGATGATGAAATTGCTGAAGAAGAAGCA
GAAGAAGGAAGCATTTCTGAAATATGAATGAAAAAAATTACATCTTTAGA
AAAAGAGTTATTAGAAAAAAGCCTTGGCAGCCGTCNGGGGGAAGTGACGC
ACAGAAGAGACCAGAGAATAGCTTCCTGGANGAGACCCTGCACTTTACCC
ATGCTGCTGGATGG
nt: 641
SEQ ID NO: 328
CCGGGTTTTAGTATTTAACCAAGAGCCTTTTAAATATTGAAAACCCATAG
TTCAGAAAATGTTAGTATTGCTGCCCTTCTTCACATAAATTTTTTTTTAA
ATTATACTATTATTTTGCTTAATTTTATATTGGGTTAAAACAACCTTCAA
GAAGGTTAACTAGGAAAGAAGACCTTTTTGTTTTATTTTTACTATTTATA
TATAGAAGACAAATCAGCATTTGGTGATAGTTTTACATGACCAGTTATCA
AACGGTCATAGTATGAAGTGTGCAGTTGTTCATTATTAGTAAATTATGTT
TGATTTTTAAACTATTTAGTACTAATAGTTGAGATGAAAACTGAAGAAAA
ATGCCAATGTGACGTTTGTGTATAGCTAGCCTTAAAAAACTTCCCATGTT
TTTAGGTGACTTTTTTCCCCCTCTTAGTACTCTGGAGAAACAATGAAGAT
GGGCCATCTCAATTCCAGATGTAAACAAAAAGTAATTTTTATTTCAACAT
TTAATGTAACTGCTATTATTGNGGATTCTTGNCTTGNGTATTTTCTTTCC
CTTATTCAAGTAATATAGAATAACTTTCCTTAAAATGATTTGATCCAAGA
TACGTCATTTCTGTATTGGCAAAATGCCNCTATTAAAGTGT
nt: 132
SEQ ID NO: 329
GTTAAAGTGATACATTTTTATACCAAATGTGTTTATTTTTTTGTGCAAGT
AATCCTTAAAATTGCAATTGTATTAGGTGTTAAAATAAAGTTTTTAAAAA
ATTAAAAAAAAAAAAAAAAAAAAAAAAAAAAA
SEQ ID NO: 330
GACAACCTTAGCCAAACCATTTACCCAAATAAAGTATAGGCGATAGAAAT
TGAAACCTGGCGCAATAGATATAGTACCGTAAGGGAAAGATGAAAAATTA
TAACCAAGCATAATATAGCAAGGACTAACCCCTATACCTTCTGCATAATG
AATTAACTAGAAATAACTTTGCAAGGAGAGCCAAAGCTAAGACCCCCGAA
ACCAGACGAGCTACCTAAGAACAGCTAAAAGAGCACACCCGTCTATGTAG
CAAAATAGTGGGAAGATTTATAGGTAGAGGCGACAAACCTACCGAGCCTG
GTGATAGCTGGTTGTCCAAGATAGAATCTTAGTTCAACTTTAAATTTGCC
CACAGAACCCTCTAAATCCCCTTGTAAATTTAACTGTTAGTCCAAAGAGG
AACAGCTCTTTGGACACTAGGAAAAAACCTTGTAGAGAGAGTAAAAAATT
TAACACCCATAGTAGGCCTAAAAGCAGCCACCAATTAAGAAAGCGTTCAA
GCTCAACACCCACTACCTAAAAAAATCCCAAACATATAACTGAACTCCTC
ACACCCAATTGGACCAATCTATCACCCTATAGAAGACTAATGTTAGTATA
AGTAACATGAAAACATTCTTCTNCGCATAAGCCTGCGTCAGATTAAAACA
CTGAACTGACAATTAA
nt: 370
SEQ ID NO: 331
AAAGAGCTCCCAAATGCTATATCTATTCAGGGGCTCTCAAGAACAATGGA
ATATCATCCTGATTTANAAAATTTGGATGAAGATGGATATACTCAATTAC
ACTTCGACTCTCAAAGCAATACCAGGATAGCTGTTGTTTCANAGAAAGGA
TCGTGTGCTGCATCTCCTCCTTGGCGCCTCATTGCTGTAATTTTGGGAAT
CCTATGCTTGGTAATACTGGTGATAGCTGTGGTCCTGGGTACCATGGCTG
GTTTCAAAGCTGTGGAATTCAAAGGATAAATTAATGAAGAAAACAAGCGG
AGCTGAAGAAGAAAGTACAATATGGTGCTGTCTTCCTAATGAAATAAATT
CACTAAATGGACATTAAAAA
SEQ ID NO: 332
AGACTCGAGCAAGCTTATGCATGCATGCGGCCGCAATTCGAGCTCGGCCA
CTTGGCCAATTCGCCCTATAGTGAGTCGTATTACAATTCACTGGCCGTCG
TTTTACAACGTCGTGACTGGGAAAACCCTGGCGTTACCCAACTTAATCGC
CTTGCAGCACATCCCCCTTTCGCCAGCTGGCGTAATAGCGAAGAGGCCCG
CACCGATCGCCCTTCCCAACAGTTGCGCAGCCTGAATGGCGAATGGAAAT
TGTAAGCGTTAATATTTTGTTAAAATTCGCGTTAAATTTTTGTTAAATCA
GCTCATTTTTTAACCAATAGGCCGAAATCGGCAAAATCCCTTATAAATCA
AAAGAATAGACCGAGATAGGGTTGAGTGTTGTTCCAGTTTGGAACAAGAG
TCCACTATTAAAGAACGTGGACTCCAACGTCAAAGGGCGAAAAACCGTCT
ATCAGGGCGATGGCCCACTACGTGAACCATCACCCTAATCAAGTTTTTTG
GGGTCGAGGTGCCGTAAAGCACTAAATCGGAACCCTAAAGGGAGCCCCCG
ATTTAAAGCTTGACGGGGAAAGCCGGCGAACGTGGCGAGAAAGGAAGGGA
AAAAAGCCAAANGGAGCCGGCGCTAGGGCCTGGCAAGTGTACGGGCACGC
TGCGCGTAACCACCCACACCCCGCCGNGCTTAATGCCCCNTTCAGGGCGC
GTNCTGATGCCGNATTTTNTCTTACNCATNTGTGCNGGNTT
SEQ ID NO: 333
TAAAATAATGGCAAAAAACAAACAAAAAACAAGTTCTCTAAACAGAAAGG
AAATTACTAAAGAAGGAATCTTGAAATAACAGGAAAGAGGAAATACCACA
GTAGGCAACATTATGGGTAAATAAAACAGACTTTCCTTCTTTAGTTTCCT
AAAATATGTTTGATGATTAATGCAAAAATTACAATATTTTCTTATGTAGC
ACTAAAGGTATGTAGAGAAAATATTTAAGATAATTGTACTGTAAGCGGGA
GATGACAGTGACATAAAGGCAACGTTTTTATACTTCACTCAAACTTTATG
TATTAATGTAATCCATAAAGCAACCAAAAAAGCTATACTAAGTACATTCA
AAAACACAATAGATAAACCAAACAAAATTCTAAAGGATGTACAAGTAACC
CACTGGAAGCTGCAAAAAATGTAAACAGAAACTAAAAACAGAGAATAAAT
GAAAAATTAAAAACGAAATGGCAGACTTAGGCCCTAATATACAAATTATC
ACATTAAATATAAATGGTCTAAATACACCAACTGTAAGACAGAGATTAGC
AAAGTCGATTTAAAAACATGACTCAACTACGTGCTGTCTACAAGAAACTC
ACTTCAAATATACCAAGATAGGAAGGTTGAAAGTAAAACGATGGAAAAAG
ATGTATCATGTGAACATTAATCAAAGGAAAGCAGGGGTGGCTATATTAAC
ATCAGGTAAAATAAACTTT
nt: 603
SEQ ID NO: 334
TGAGGNTGGTCATGATGCANAAGCTACTCAAATGCAGTCGGCTTGTCCTG
GCTCTTGCCCTCATCCTGGTTCTGGAATCCTCAGTTCAAGGTTATCCTAC
GCGGAGAGCCAGGTACCAATGGGTGCGCTGCAATCCAGACAGTAATTCTG
CAAACTGCCTTGAAGAAAAAGGACCAATGTTCGAACTACTTCCAGGTGAA
TCCAACAAGATCCCCCGTCTGAGGACTGACCTTTTTCCAAAGACGAGAAT
CCAGGACTTGAATCGTATCTTCCCACTTTCTGAGGACTACTCTGGATCAG
GCTTCGGCTCCGGCTCCGGCTCTGGATCAGGATCTGGGAGTGGCTTCCTA
ACGGAAATGGAACAGGATTACCAACTAGTAGACGAAAGTGATGCTTTCCA
TGACAACCTTAGGTCTCTTGACAGGAATCTGCCCTCAGACAGCCAGGACT
TGGGTCAACATGGATTAGAAGAGGATTTTATGTTATAAAAGAGGATTTTC
CCACCTTGACACCAGGCAATGTAGTTAGCATATTTTATGTACCATGGNTA
TATGATTAATCTTGGGACAAAGAATTTTATAGAAATTTTTAAACATCTGA
AAA
nt: 71
SEQ ID NO: 335
ATTTATCTAATATTTGGTTTAATAAAATGTGAATAATGAAAAAAAAAAAA
AAAAAAAAAAAAAAAAAAAAA
nt: 622
SEQ ID NO: 336
TTTTTTTTTATTTTTTGAGAATGGAGTCTTGCTCTGCCGTCCAGGCTAGA
GTTCAGTGGTGCGATCTCAGCTCACTGCCACCTCACCTCCTAGGTTCCAG
AGATTCTTGTGCTTCAGCCTCCTCAGTAGTTGAGAATACAGGAACACGCC
ACCACGCCTAGCTAATTTTTGTATTTTTAGTAGAGATGGGGTTTCACCAT
GTTGGCCAGGCTGGTCTCAAACTCCTGGCCTAAGTGACCCACCTGCCTCA
GCCTCCCAAAGTGCTGGGATTATAGGCGTGAGTCATTGTCCCCAGCCGGA
TGTTTTCATCTTGATTTGCCTTAGTTTCTAAATCTCATCCTCTCCATTTT
CTCCTGTTAGTAGTCACAGAGAACCAAATTCTGTCAAGTTATGAAACTAA
AGTCTCTCTTCCACAAGTCTTCCTGTGTTCTGCCTCAAGTGAACTTGAAA
GAACATCAGTTTGTGGGAAGGTTGAAGACCGAATGATCTGCTGGGAAATC
ACTGAGGCATTGCCATTCTCTTGAGGAATTTCATTTTCATCGAAGTTTCG
GTTTATATCCCTTTCTTGGTGAGTACTATTGCTGTTATGTAAATTAAATG
AGTCGTCATCCTTCTTNTGAGC
nt: 501
SEQ ID NO: 337
GTGAAATCACTTTCATGGATTATTAATGGATTTAAGAGGGCATCAATCAG
CTCAACTCAAGATTTCATAATCATTTTTAGTATTTAGATTGTGCCTCAAA
GTTGTAGTACCTCACAATACCTCCACTGGTTTCCTGTTGTAAAAACCTTC
AGTGAGTTTGACCATTGTGCTCTTGGCTCTTGGGCTGGAGTACCGTGGTG
AGGGAGTAAACACTAGAAGTCTTTAGTACAAAACTGCTCTAGGGACACCT
GGTGATTCCTACACAAGTGATGTTTATATTTCTCATAAAGAGTCTTCCCT
ATCCCAAGGTCTTCATGATGCCAGTAGCCATATATGATAAATTATGTTCA
GTGATAACTTAGTTATCAGAAATCAGCTCAGTGGTCTTCCCCGCCATGAT
TCACATTTGATGAGTTTTTAAAAATCAAAGTGATTTTGAAAATCTCTAAT
GGCTCAGAAAATAAAAACATCCAGTTTGTGGATGACTATATTTAGATTTC
T
SEQ ID NO: 338
TTGTGTTTTTAGGACTCCTTATCTAAATTAAGGCAGAGAAGTTACAGTAT
TTATATCTGCATTAAATCTCAATTCCAGAAAAACCTTTTGAAAAATTATT
TAATCCTCTGGAAACTATTGATATGATACAGGAGAAATTTTCAGAAGTTT
ATTGAATAATTTAATATCATTTAATAGGACACTCTGGCTTGTATATAAGC
AGATACGTTACTCAGACTTCTTGGCTGTACTCTAAAATAATATATGTACT
AGTCTCCTAAATATTACTAGCTCACCTTTCAAAATGCATACTAATATTTC
AATGTCTTTCTTCAATTTGAAAAGCTCTTGAATATCTACTTGTGATAGCC
CTAAGAGCTGAGATAATTATTTCCAGGAGGTTGAATCCCTGATTCTTAAC
TGTTCAGCAATGCATAAGCAAGAGAGAATATGACATAAGAGGACCATTTC
TACATTAGCCATTTTTTTTCACAAGATACCTATGTGAATACAGGGCACCT
GGGAGGGTAAGTGGAGGACTATTTCTAACTATATTTATAAGCACATACTG
ATATTGGTGAATCAAAACCTACAGCAGTGCTTCTCAGATGGGAAGGGAGA
CAATGTGTAAGGAGATCAGGAATTCATTAG
nt: 122
SEQ ID NO: 339
CCANAATCCACTCTCCAGTCTCCCTCCCCTGACTCCCTCTGCTGTCCTCC
CCTCTCACGAGAATAAAGTGTCAAGCAAGAAAAAAAAAAAAAAAAAAAAA
AAAAAAAAAAAAAAAAAAAAAA
SEQ ID NO: 340
TTTTTTTTTTTTTTTTTTTCAGAGTCACAGATATTGTATAGCTGAGGTAA
GCATTTTACAACTTTTCAGACACAAGTAAGTACATAAATATTATTTTACA
ACCAACAATNTTTAATATTTCCACATTGAANAATAGATGTGATAATTAAA
TCTTTTATAAGGTTTTAAAAAGACATGAAACATAAACCTAATTATACATA
AAAGAAAAGAATTTTAAACAAGAGCTTATTGNGATGACATTACTCATAAC
TTTTACCTTTAAAACCTTTTCTTGGGTAGCTATTCAAAAGTAAAGACCAC
AAGTTTTGTTGCCCANATTTCTTATGTTTNGTATATTTAAGCTCTTTATT
TATTGAACAGATGNGTCATTAATTCATTNGGAGCATTACTATTATCAGTA
AAATTTGATTTTTTTTTCCCCTCAGTCATAGGTAAATCAGCTCCACCTGG
AATTTCTAAGGACCCAGTTTTAGTCAATATTTTCAAGTAATCATGACCTC
AGAAATAGTCTTAATTAAGATAACAAATATTAGCCATCAAAATGGAACCA
AGACAAGATTCTAATGTTTGTAAACAGTCAATCCATATTTATGAATATTA
GCATATATTGGNGAATAGTTAAGGCAAAAGGGTCTAGCAG
nt: 667
SEQ ID NO: 341
TTGTGTTTTTAGGACTCCTTATCTAAATTAAGGCAGAGAAGTTACAGTAT
TTATATCTGCATTAAATCTCAATTCCAGAAAAACCTTTTGAAAAATTATT
TAATCCTCTGGAAACTATTGATATGATACAGGAGAAATTTTCAGAAGTTT
ATTGAATAATTTAATATCATTTAATAGGACACTCTGGCTTGTATATAAGC
AGATACGTTACTCAGACTTCTTGGCTGTACTCTAAAATAATATATGTACT
AGTCTCCTAAATATTACTAGCTCACCTTTCAAAATGCATACTAATATTTC
AATGTCTTTCTTCAATTTGAAAAGCTCTTGAATATCTACTTGTGATAGCC
CTAAGAGCTGAGATAATTATTTCCAGGAGGTTGAATCCCTGATTCTTAAC
TGTTCAGCAATGCATAAGCAAGAGAGAATATGACATAAGAGGACCATTTC
TACATTAGCCATTTTTTTTCACAAGATACCTATGTGAATACAGGGCACCT
GGGANGGTAAGTGGAGGACTATTTCTAACTATATTTATAAGCACATACTG
ATATTGNTGAATCAAAACCTACAGCAGTGCTTCTCAGATGGGAAGGGAGA
CAATGTGTAAGGAGATCAGGAATTCATTAGTCACCTTTCAGATGGTTTAA
TGCATACAGCTGTACCG
SEQ ID NO: 342
GGAGTTTGAGCAGATCCTTCAGGAGCGGAATGAACTCAAAGCCAAAGTGT
TCCTGCTCAAGGAGGAACTGGCCTACTTCCAGCGGGAGCTGCTCACAGAC
CACCGGGTCCCCGGCCTTCTGCTCGAGGCCATGAAGGTGGCTGTCCGGAA
GCAGCGGAAGAAGATCAAGGCCAAGATGTTAGGGACACCAGAGGAAGCAG
AGAGCAGTGAGGATGAGGCTGGCCCATGGATCCTGCTCTCCGATGACAAG
GGAGACCATCCCCCACCCCCGGAGTCCAAAATACAGAGTTTCTTTGGCCT
ATGGTATCGGGGTAAAGCTGAATCCTCTGAGGATGAGACCAGCAGCCCTG
CACCCAGCAAGCTAGGGGGAGAAGAGGAGGCCCAACCACAGTCTCCAGCT
CCTGATCCGCCCTGTTCTGCCCTCCACGAACACCTTTGTCTGGGGGCCTC
AGCCGCCCCAGAGGCCTGACTTAGGGGTCTGGCTGTGGAAGGATGTGTGG
CCTCAAATGAGGACAGGGCTCCCGCCTTCACAGCCCTCGCCAGGGGTCTG
CCCCAATCCTGGCCTGCATCAGGCAAGGACGGGGTCTCAGC
nt: 642
SEQ ID NO: 343
GCAAGTCTTCAGTATGTACATTTATCCCCTAGAAGAAGAAAAATTAGTTG
TGCATGAAAAAGAAACATTAACTGCAAAGCTAAATGCTCACACTCTAAAT
CAGTGCTCTCCAAAGTACAGCAGGCGGGAAAAGAAAATGGTAGATTTTTT
TCTTCCAATTACTTTAACTTATTCTTTTTAATGGACACTTCATACATAAA
TATATTCACAATATATTAATATATACATAATGTATAAGCATACATATTGA
ATGTGCAGTCAAAAAATGTACTAATGGAATGCTCTACCAAAACAAGTTCA
CGTTCATCTGTAAAATGGGAATAATATTTTTAAAAGGCATACAGTCTGAA
CATTTTTAGATTATTCATAAAATCTATTCAGAAAGTTAAACTAAAAAATT
TAACGTATGCCTATAACAAATTTTGTACTTAATGTAATTGNTTTTCATCC
TGAGATCTAATATCCTCGTTTTTAAGTAGAGCCACTTGTTTGCTACAGTT
TAGTCAAAACGTTAACATTAGATGGGTAAAGTAATATGAAATCTTTCTAC
TACTCCAAAATAGAAAACAGAACATTAAAAAGATAAAAATTCAAACATAC
TTACCAGTAGATTTTCAACTGNGCAAAAGCTCATTGCATGGG
SEQ ID NO: 344
GTTTTCCACCGTGAAGAGAACATTTCCTCTGGGAATGACAAAGCCCTCAG
GAACNGCTTTTATTTCTATTGGAAGATGCCCATCATACTTCTGGCAGGAT
AAAATGATAAATTTATTTATTCAACAGATGATACTCAATTCCCTGCTGTT
TTACTAAAGGTTCTTTACGTTTTATAGAAGCTAAATTTACTGTCATAGAA
ATTGCAATTGTAGATGTTACTGTAATCTAGTCAGAATATCCTTATCCTTC
TAAAATAAAACTAGTTAAAATTATTAACATACGTACTGATATTAATTTTT
AAGTTTAATGCTGCCACGTGCTTCTGCTAAGAACATTTATCACTACAAGT
GGCAGAAAATTCCAAACTCATCAAAACCAAACTGTTGCTTCTTCCCTGCT
TTTTCAGAAAATGAGAAAGGATGACTTTATTCCAACATATTCTAAAAGTA
TTCCAAGAACACTACCTTTATTCTAAATTCGTTATTTTCACAAAATAAAG
GCTGCAGATTGAAAGATAAAGGATTGCTATTAAAGAACAAAAGAAAACAA
AACCGAGAGAGAAGGAGAGCTAGGGAAATCCCTGCANAANAACCGAATAN
GGTCCCTCTATTCTGGGCCGGGGCCTGAAACTATGAAACAGGCCAACACA
GAATCTTGGCA
SEQ ID NO: 345
CCTCTGACTCGCTCAGCTCACCCACGCTGCTGGCCCTGTGAGGGGGCAGG
GAAGGGGAGGCAGCCGGCACCCACAAGTGCCACTGCCCGAGCTGGTGCAT
TACAGAGAGGAGAAACACATCTTCCCTAGAGGGTTCCTGTANACCTAGGG
AGGACCTTATCTGTGCGTGAAACACACCAGGCTGTGGGCCTCAAGGACTT
GAAAGCATCCATGTGTGGACTCAAGTCCTTACCTCTTCCGGAGATGTAGC
AAAACGCATGGAGTGTGTATTGTTCCCAGTGACACTTCANAGAGCTGGTA
GTTAGTAGCATGTTGAGCCAGGCCTGGGTCTGTGTCTCTTTTCTCTTTCT
CCTTAGTCTTCTCATAGCATTAACTAATCTATTGGGTTCATTATTGGAAT
TAACCTGGTGCTGGATATTTTCAAATTGTATCTAGTGCAGCTGATTTTAA
CAATAACTACTGTGTTCCTGGCAATAGTGTGTTCTGATTAGAAATGACCA
ATATTATACTAAGAAAAGATACGACTTTATTTTCTGGTAGATAGAAATAA
ATAGCTATATCCATGTACTGNAGTTTTTCTTCAACATCAATGGTCATTGN
AATGTTACTGATCATGCATTGGTGAGGNGGTCTGAATGTTCTGACATTAA
CAATTTTCCAT
nt: 115
SEQ ID NO: 346
AAACTTTTGTGGCAACAGTGCACTAATTTGGATAATGTTTGTTCCCAATA
AATTAAGAGCCAAATTGTAAAAAAAAAAAAAAAAAAAAAAAAAAAAAAAA
AAAAAAAAAAAAAAA
nt: 634
SEQ ID NO: 347
GCCAGGCTTTGTGAATTACAGGACATTTGAGACAATCGTGAAACAGCAAA
TCAAGGCACTGGAAGAGCCGGCTGTGGATATGCTACACACCGTGACGGAT
ATGGTCCGGCTTGCTTTCACAGATGTTTCGATAAAAAATTTTGAAGAGTT
TTTTAACCTCCACAGAACCGCCAAGTCCAAAATTGAAGACATTAGAGCAG
AACAAGAGAGAGAAGGTGAGAAGCTGATCCGCCTCCACTTCCAGATGGAA
CAGATTGTCTACTGCCAGGACCAGGTATACAGGGGTGCATTGCAGAAGGT
CAGAGAGAAGGAGCTGGAAGAAGAAAAGAAGAAGAAATCCTGGGATTTTG
GGGCTTTCCAATCCAGCTCGGCAACAGACTCTTCCATGGAGGAGATCTTT
CAGCACCTGATGGCCTATCACCAGGAGGCCAGCAAGCGCATCTCCAGCCA
CATCCCTTTGATCATCCAGTTCTTCATGCTCCAGACGTACGGCCAGCAGC
TTCAAAAGGCCATGCTGCAGCTCCTGCAGGGACAAGGACACCTACAGCTG
GCTCCTGAAGGAGCGGAGCGACACCAGCGACAAGCGGAAGTTNCTGAAGG
AGCGGCTTGCACGGCTGACGCAGGCTCGGCGCCG
SEQ ID NO: 348
GTTGCCGGGTCCTGTGATAACTCTGTTTAACATTTTGAGGAACTGTTGAA
TGGTTTTTCACAGCAGCTGCCTCATTTTTTATTCCCATCAGCAGTACTTC
TTGGTTCTAATACCTCCACGTTCTCGCCAACACTTGTTGTTGTCTGTAAT
TTCGTTGTTAGCCATCCCAGTGGGGATGAAGTAGTATCTTACTGTGGTTT
TCAGTTGCGTTTCCCTGATAATTAATGATGGTGAACATCTTTTCATGTTC
TTGTTGGCCATTTGTATGTCTTCTTGGGAAAAAAAAAATGTCTGTTCAAA
TCCTTTACAAAGTATTTATTTTTTATGTCAACAATATAACCACTCAGTAC
ACTGCTTTTTANACAATGATCTTTTAAAGGTTTGTTTACAACATTTAGCA
CTTGAAATTTTAAGGTTATGCCCTCAAAAAAATTGCTGAGGGAGCTAAGC
TATGAAGATGCAAAGGCATAANAATTATACAATGGACTTTGGGGGAATCC
AGGGAAAGGGTGGGAGGGGGGTGANGGA
SEQ ID NO: 349
TCGACTCTGATTTTTTTTTCTCCTTCCTCGCAGCCGCGCCAGGGAGCTCG
CGGNGCGCGGCCCCTGTCCTCCGGCCCGAGATGAATCCTGCGGCAGAAGC
CGAGTTCAACATCCTCCTGGCCACCGACTCCTACAAGGTTACTCACTATA
AACAATATCCACCCAACACAAGCAAAGTTTATTCCTACTTTGAATGCCGT
GAAAAGAAGACAGAAAACTCCAAATTAAGGAAGGTGAAATATGAGGAAAC
AGTATTTTATGGGTTGCAGTACATTCTTAATAAGTACTTAAAAGGTAAAG
TAGTAACCAAAGAGAAAATCCAGGAAGCCAAAGATGTCTACAAAGAACAT
TTCCAAGATGATGTCTTTAATGAAAAGGGATGGAACTACATTCTTGAGAA
GTATGATGGGCATCTTCCAATANAAATAAAAGCTGTTCCTGAGGGCTTTG
TCATTCCCAGAGGAAATGTTCTCTTCACGGTGGAAAACACAGATCCAGAG
TGTTACTGGCTTACAAATTGGATTGAGACTATTCTTGTTCAGTCCTGGTA
TCCAATCACAGTGGCCACAAATT
SEQ ID NO: 350
TCATTTACATTAATACTCAAAACTGCTCGATTAAGCAGGTGCTGTTCTTA
TCGCCATTTTGCATATGATGAGAAAGGGTAAGGTCACCCAGCTAGTATTT
GGCTCACAGCAGGCCTTAAGACTTGGTTTGTGTGACTCATCAGTCCACGC
TCCTAAAACCACTAAGTTGTTCTACCCTTTAATGTTGAATTAACATTGGA
TAGTGTTCAAGTTTANATGGGTGGGTGAGGGCCCAAGGACCTTTCAAACT
CAGATCTCTTATTTAATAACCTGGTCCCAGATCCATTCCTCTGTCGAAGA
GGAAGTCATCCTTCAGTGGCTATTCATTGTGGGGTTAAGAGCGCAGACTA
TGAATTCAGTCTTTTTGGGTCCCAGTTTGCCAGACCTTGAGTGAGTGCCC
CGAGTTTACTTACTTGTAAAGGTAGGTGGAGGTAATATAATTAAATAAAC
TTAAAAAACTAATTAAAAACAAAACAAATGAACTAAGGTCTTAGGATATC
TGGCGTCTATTTTGCGCCAAATCACATAATGTCTATTGTTGTGTGTTGGA
CTATAGGATTGTCCTTTAACAGGGAAGGGTTTATTTCTGTAATCAAGTCT
GTCAATATTATGACCATGTTGATAATAGCTACCTTTAATTGAGGGCTTCC
ATGTGCCAA
SEQ ID NO: 351
TCAGTGGAAAAGGGCAGGTTGAATCAAGGTGAATCAATCTGAAATTGAGC
ACACCTGCCTGCCATCGCTGTTCCTTCAACTGAGTGCTGCACATCATGGG
CTCTGTCTGTGAGAGAAAAATCCCGGTGCTTGGTGTCCTTGCATGACATG
GAGTTTTGCATGTAGATCAATTTAAAATGTACCTCTTGTTTACATAATTT
GCATAATTTTAAAAGATAATGTTGCCAAACTTTGGAAATGTTAATGTTCA
NACTGAAAATCTCCACTACATGTAACTTTCTTCCTCTGGATCAGTGGCAT
GGCTTATAATCCCAGCCAGTGGTTTGAACTGTTCCAGTGTCAACTGCCAT
GTGCTCTGCTTCAAGGGGGAACTAGCCTTTTGTGAATTTTTTGTACATAA
GTATTTGTTACAAATATTTTAGCAAATGCTTTCTATTTCTCTTGCTTGTG
CATATCTTGGCTGGCGTTACAGAAAAATAGTGTAAACATTATTTCCTTAC
CGGGGAATGAGGGTTTT
SEQ ID NO: 352
AGCACCTGGCACAGAGTAGTAGCTAACACAGATGTTAATTTTGCTGCGTC
AAATGTTTTCACTTTGAATCTCTCTTGAGTATTGTTCTCCTTATTGATTA
CATGATGACATCCTGTTTTCTCTCCCTGACCTTTACTGTTTGTTTAGAAA
AAAAAAAAAAAAAAAAAAAAAAAA
SEQ ID NO: 353
CAGAGAGCTTGTTCCCTCCCTCCCTGTGCATGCAAACAAGAGGGCATGGG
AGCACACAGAGAGATGGCAGCCACCTACAAGCCAAGAGGAGAAGCCTCAC
AATCAAACTCTCGCTGCTGGCGAGAGTCTTGGACTCTGTCTTGGACTTCC
AGCCTCCAGACTGTGAGAAACAAATTTCTGTTGTTTCAGCTTCTCAGTCT
CTGGTGTTTTGTTATTGCAGCCTGAGAACACAGCTGTACNATTATNAGGG
AAACAGAAAACACTGATACTTAACAATGCTAATGCAATTATTTATTTGCT
TTTCAGTCTCTACAAAACGTTCTAAAACACTAATCTAAATATTAACAGTA
AAATATTTGCATAACTAATGGAAACTAAGAAATCATATGACCAATATTTC
ACTTATTGGTAATCTTACTCTACTGATTTCCCCCCAGACTGTGATTTTTG
AACTTCCTTGCCTTTCTCCTGTCTTTCTGNGTTTATTCATGGAATTCCAG
TTATCTGGGCTTGAAATTGCAGGCTCTCCTAACTTAAGCAAAATCTGACA
GATCAGCAAAATGAGATAAATGTTTCTTTTTTCTTTCTGACTGCATTAAA
TCAGATACAACTCAGCATTAAAAAGCTATCTTTGNAAAATGNTGGTACTA
ATAAATTAGTCTTA
SEQ ID NO: 354
CCAGTTCCACATTCAGTGAAGTCATGAACTTGAAATTGGCCATGATCAAA
AAGTATTTAAATCACAGAAGTTGCAAATGCCACAAATCAAGGTCTTTTTC
TCTTGGAGAACCTGTTAAACATTTACCAACTCACGACCGCCATGCACCCA
ATACTGCAATAGGTCTATAGATGCAGATACTGTCTCCATGAATCTTATAG
GCTAGAAAGGAAATAGATAAGTAGTCCTACCAGAAGAACATGATGAAGGC
ATTTGTGGTAAACAGAATGATGGCCCCCCAAAGATGTCCACATCCTAATC
CCTGAAGCCTATGAATATACTACTTTACTTGGCAAAAGGGACTTTGCCAC
AGGTTTTTAATTAAGGACCTTGAAATAGAGAGATTATCCTGGATAATCCA
GATGGCCCCAGTGTAATCCCAAGGGTCCTCACAAAGGGTAGGAAGGAGAG
CCAGAGTCAGAGAAGGAGACGTAGCAATGGAGGCAGAGGTCANAGAGAGA
TCTGCAGATGCTGCTGTGTTGGCTTTGAAAATGAGGAATGCAGGTGACCT
CAANGNGCTAGATGATGCAAGGAAACAAATAATCTCCTATGAACCCTAGG
ATGGGCATTATTATGAGTCCTATTTTATAAACAAGGAACTGACNTCCAGA
AAGATAAATGC
nt: 626
SEQ ID NO: 355
GGCAGAGGTTGCAGTGAACTGAGATCATGCCATTGCAATCCAGCCTGGGC
AACANGAGTGAGACTCCATCTCAAAAAAAAAAAAAAAAAGACAAGAGTNT
CCACTCTAAACACTTNTATTCAACATAGTCCTGAAAGTCGTAGCCACAGC
AATTTAACAAGATAAAGCAATAAAATGTATTCAAATAGAAAAAGAGGAAG
TCAAATTATCTTCACTGGNGATATAATTCTCTACCTGGGAAACTTCACCG
AAAAAGATTTCACCAAAAGATTTCTAAGCCTAAATAATGACTTCAGCAAA
GTCTCACCATACAAAATCAACATACACAAATGAGTAGCATTTCTGTGCAC
CAATAATATTCAAGCTGAGAAAAAAAGAACATGGTTCTATTTACAATAGC
TACAAACAAAAAAATATGTACCTAGTAATACATTAAATCAAGGNGGTAAA
ATATCTNTACAACAAGAACTACAAAACTGCTGAAAAAAAATAGAGACACG
CAAATAAGTAAAAAGGCACTCCATGCTCATGAATTTAAAGAATCAATATA
ATTAAAATGTCCGNGCTGCCTAAAGCAACTTACAGATTAAAGGCTATTTC
TCTCAAACTATAAATGCACCTTTTTA
nt: 585
SEQ ID NO: 356
GTCATTGCTGGGTGGCGCCAGCCCTCAGACTTGCCTCTTTGCAGTAGGAA
GAAGGCCTCCCCACATACCTTCCCACACTCATCACCTTAAGCCAGACTCG
GTGTCCAGTGAATATGACCATCTCTTGCCCATTTTCTAATGAGTGTTTTC
ATTAATGAGTTATAAGAATGTGGTGGGTAAATCTATGGGCTTTGAACTAG
TGAATCAACTTGGTTTCAGAATCTGGCACTGCTACTTACTAGTGAATTTA
AGCAAGTTATTTCACCTTTCAGAGTGTCAGTTCCCTCATGCATACAAGGA
AGATAAAAAATAATGTNTACNAAAGTATTGGAGTAATTAATACATGGAGA
ACTACATGTAAAGCGTTTAGCATGATGTCTGACATATTAAGCATCCAATA
TTAGTNGCTTGCAGAATTATTAGTAAAAGAGATTGCTTCTGAAAGCCATT
CCAATTCTTAAATTTTATAATGCCACATTTGAGGTCACCTGAAGTCGTGT
ATAACATGTGTACATTTTTGCGATTTATTTTTTCAATTCCCANATTAAAG
GCATAGAGATATCCTAGCNANGGACTCCAAGTGTG
nt: 560
SEQ ID NO: 357
GTAATTGCAGCCTGGGCAACGGAGTGAGAGACTGTCTCAGGAAAAAAAAA
AGAAAAAAAACTACTGAGGTAGTTGAATATATCCTCCATTCCCCATTTGT
GGATTAGTTAGTAAATGGGGCATCTTAGGGTTTAAATATGTCCAGGGTCA
CTGAGGATCAGATCCTAGGGTTCCTTTGACTCAAGGCTTTTGTCTCAGCA
AAACGTCACCTTCCAGCAGGAAGGCTTTCTCAGGCAAGTAGCAGGGTGGC
TACTATGTATCGCTTCTTTATTTTTTCTTTTTTAAAATAATGCAGGCACC
GTGCGCATAATTTAAAAAATCAGTGCTAAAACCCTTAAAAAAAAAAAGCT
GTTCTCATCTCCTGTCTTTCTTTTTTTTTTCTTTTTATTTTTTTCTTTTA
TTATTATTATACTTTAAGTTTTAGGGTACATGTGCACAACGTGCAGGTTT
GTTACATATGTATACATGTGCCATGTNGGTGAGCTGCACCCATTAACTCG
TCATTTAGCATTAGGTATATCTCCTAATGCTATCCCTCCCCCCTCCCCCC
TTTTTTTTTT
SEQ ID NO: 358
GGGAATGTCTTAGGCACTGGGACTGTAAGTGCAAAGACCCTGTGGCACAA
GGGAATGTTAATTATCTACCTTTCANAAACTGGAANAAGGCCTAGCCTAG
AGCATTGAAAACAATAAGGGAAAGGAGGAGTAAGGCTGGANAGATAGGAA
TGGTTTAAAGTCTTTGTTAAAAATTTTTTTAAAAAAATCTTTATCACAAG
AAGAGGATTGGCNTGATCAAATTTGACTTTTAAAAANATTACTTGGGTTG
GGCATGATCAAATACTACTTAGGGAGATTAGTTTANATGATAATGGCATT
CTGGACCANAGTGGAGTCAGAGGTGAAAAGAGGTAGATATTCCANAATTG
AGGGATTTGTGAGGTGAAATCATTTGTTACAGATATTAAAGGATAAGGAG
CTTTGTCAAAGGGGATCTTAAGTTTCTGGTATGGTAACTGGGTTAGAGAG
CCCTGGAACATGACCAGCTTTAAGGGAAGAGAGCTTGAGCTCTGTTCTTG
TTAAGCTCAGTTTGAGATCTTTGTGGAATCAAGTGGAGAGGTCTAAGCAG
GGAACTGGCTTGGCTAGGCTGTAAAGATGAATCTGAGAGTCCCAAGAATA
TGGTAATTATTAATAAAAGCCTTAGGTANATGAAATTGTTTTGGG
nt: 509
SEQ ID NO: 359
GCAAATCTACACATTTGATTAAATGATAGGGAACTATGCACACACATAAT
ACATATAATGCTAGTTTCTTGGTTTTGATATTGTACCATAGTTATGTAAG
ATGTAACCATTGGGGGAAACTGGGTGAAGGCTACATGAGACCTCTCTGTA
CTTAATCTTTGCAACTTATGTGAATCTATAATTATTCCAAAATAAAAAGT
TTTAAAGAACCTAAGTATCCTTATTACTGAGGGTCATCGTGCTAGACAGC
AAGGTTGGGCCAGAGCTTCTAGTTATTTAAAATACTAAATACCAGCCTGG
GCAACATAGCAAGACCCTGCCTCTACAAAAAGCAAAAAAATTAGCTGGGC
ATGGTGGTACATGCCTGTGGTCCTAGTTACTCTTGGAGGAGTCTGAGGTG
GGGAGCTTGAGCCTAGGAGTTTGAGGCCGCAGTGAGCCTTGATTGTGTCT
CTGTACTCCAGTCTGGGCCACAGAGCAAGACCCGGTCTCTAAAAATAAAT
AAATAAATA
SEQ ID NO: 360
ANTGCACTCCAGCTTGGTGACAGAGGGAGACTCCATNTTAAAAAAAAAAA
AAAAAAAAAAAAAGGGAGTAGCTTGAAGCCACATAGTAGTTAGTGGTAAA
GGCCACCCCTTTTCCCACAACTCACACCAGCACCACAAGCTAGCCTTTNT
AATTTCCAAGCCAGTGCCCTTTCAACGCACACACCCCTGTGTCAGTTCCC
TTTCTGCTGCAAGCTCTCTGGAGGCAGATACTGTTGAGTCCCTGGCCTGC
CTATGAGAACGGCTCATGATCTCTATTTCTTCTGCTTAATGACCATCTCG
AAGTAACAAGTTTAGCCTAAAATAAACTTGCTAAGTTAGCAAAGGAAGTC
CTTAGCAGCCACCATTTCTCGATTCCTCCATCACCTCCCCTGCCCCTCAA
CTCCCTCATTTCTCCCAAGATATGGGCTCCAGGCTGGGCGCGGTGGCTCA
CGCCTATAATCCTAGCACTTTGGGAGGCTGAGGTGAGCAGATCACTGAGG
TCAGGAGTTCG
SEQ ID NO: 361
TCNTTCGGAACGCGCC
SEQ ID NO: 362
CTGGAGGGATGGGTAGGATTTTGACAAGAGTGGTTGAAGGTATTCTAATT
CACTTAGTACCTACATGTGCGAGGCAGCATGAAGGCAAAAAAGCCTGGGG
CATGTTCAGAGAATAGCAAGTATTCTAGTTTGAGTGGCACCTGGTACGTA
TATAAGGGAATAGTAAAAGATCTGGCTGGAAAGGAAAAGTAGGGGCAGGT
TACGAAGGACCTCTGAAAGTCAGACTGTGGAACTGGAACTTTTATCAGGA
AGCAGTAGTTAGTTTTTTCAAGCAAAAGCTAATTAGAGTTGATATTTAGG
AGGATGAATCTAACAGTTGTGTGCAAGGATGCCTTCAAACTGAGTGAGAC
TAGTACTGGAGACTGGTTAAGAGACTACAACAATAACCTGAGTAAGAATT
AATACAGGCCTGACCTAGTTTTGAGTGAGTAGGATTGGAAACAAGAGTTT
TAGGTATTATAGGATTTATGCATATAAAATGGACTTGACAGAACTTGAAG
AAAGAGAAAGTGTCAAAAGGACACAGAAAGTGAGGCAGGATATCTTACAA
TGTTAAAGGAAAGGAATAATAGAAGTTAC
SEQ ID NO: 363
GGAAACATAAGCTTGTTTCAGTACACTCACGCTGTAGATTAATTCTGATA
TTACATATCTCCATCAGACTTTGTACCCTCTCTCTTCCATCCCTTACCCT
TACCGATTAGGTTGGTATTACCTAAAAATCCATAGAAAATGTCCAGGTGA
ATTGCCTTATGCTTTCTACCCCATAAGGTATAATT
SEQ ID NO: 364
CTCTGTGGTGTGAGAACACAGTGGGTGACCAAGGCTTTCCAGATGAACCC
AAGGAAAGTGAAAAAGCTGATGCTAATAACCAGACAACAGAACCTCAGCT
TAAGAAAGGCAGCCAAGTGGAGGCACTCTTCAGTTATGAGGCTACCCAAC
CAGAGGACCTGGAGTTTCAGGAAGGGGATATAATCCTGGTGTTATCAAAG
GTGAATGAAGAATGGCTGGAAGGGGAGTGCAAAGGGAAGGTGGGCATTTT
CCCCAAAGTTTTTGTTGAAGACTGCGCAACTACAGATTTGGAAAGCACTC
GGAGAGAAGTCTAGGATGTTTCACAAACTACAAAGCTGAAGAAAATGAAG
CCCTATTACTTGTTTGTAAGATTTAGCACCCTTCTGCTGTATACTGTACT
GAGACATTACAGTTTGGAAGTGTTAACTATTTATTCCCTGTTAAAATTTA
ACCTACTAGACAATGATGTGAGTACCCAGGATGATTTCCTGGGGCACAGT
GGGTGAGGAGATGGGGACAGGTGAATGGAGGAGTTAGGGGAGAGGAAAAG
TGGATGGAAGTGTCTGGAAAGGGCACCAAAAAAGTCTTCCAGGTCTGATC
CTGTTTCTTGCTCTGAGTGCTAGCTACCACTGTGTCACACTGTAACATN
nt: 655
SEQ ID NO: 365
CTCAGCTCTTGCCTGGTCACCTTGTGGCTTTTACCATCCTCATCCCCTGT
GCCACCCACATCCTGCCACTTCTGCATGGAGTTGGGGTGGGGCCATTGGA
GAAAAGAGGTTAAACAAGCAGTAATTTACTTGAGTACAGTCTTTGAGCCA
ATGAAATGCCAGTCATCATTTCCCAGGGGTACTTGTCATCTTGTCAACAA
CCCGCTGATAATGCTCCTTCAATGTGAATAGCAAAAGTAGGGAGAGACGC
TGAATGAAGAAGATGCCTACCCCTCAGGAAGACTGCTGTCCGCCTCCAGG
CCTGCATGCACACACCCATGCCCACCTGCACCCCCAGCACCACGCCCACA
CTCACTCGCACACACCCACATGCCAGTGTTTTGGGGTTGGCAGCCTGGAC
ACTGCTGAGGCAAACACAAGTCATCAAGCATAATTCTCATTCTCTCCTTC
TGTCTCTGTTTTAGTTACAGGAATTTGGTCAGTTTAGAGGATTTAATAAG
TCCGTGGAAAATTTGTTTCTGTCTCTTGCTACCCACGTGAAAAGTAAGTG
CATGCTTCATGATGTGTTTTCCCACTACCTTCCAGGCCAGCCGAGCCCAC
TGGCCANGGCCTGGCCCGGTGACCTCGGTTGACACTGTCCTCANGCCACT
CACTT
SEQ ID NO: 366
CAGAATTTCATGTTTATGCTGCACAAGGCCTGTATTTTATAATGGTGGCT
CTTTTGGACGATGACTTCCTCGATGGTGAAACTTCCAGTAATCTCCCTCA
TCATACTGAAATGATATCAGTATATCATCAGAACACCATGGAGCTTGTCA
TTTGAGGGACACAGCTTGCTTGTGTGCTTGGGAAAGAAGAGGTTTAGCAT
GGTTTCAGGTCAGTGATGAGTCCAATGATCTCTGCAAGTTCCCTTAGCTC
TGANAATTCTGATGTCATATGCACTTCTGCCGCCAGAGTTGCTGCTTACT
GGATGCGTAAGAAGAAAAGAAAAAAAAAAAAAAA
nt: 582
SEQ ID NO: 367
CTTCCATTGGGGGTAAAGATCAAACTTTAGGCGAGCCAGGTCTGTATCTC
CATTCCTGTCTCTGACTGCTTCCCTGTAGGGATTGTCTGCAAGCGCACAC
CTGCATTTTCTTGTCCACAAGTCTATGCTCTAACTCTGTCACCTGCATGG
CTGCAAATTAGCTTCCTTCTTCCTGCCCTCTTCTCTCTAGCTTGGATTTT
GAATTTGAATGGCAGGCATGGGATGTCCGTGTGTGTGTACTGCTGATGTG
TACAGCCGCTTGTTAGCGCTCTCATTGTCTTCAAATGTAAGTCATTTTGG
CTGGGTGCGGTGGCTCATGCGTATAATCCCACGCTTTGGGAGGCTGAGGT
GAGCTGATCATTTGAGGTTAGGAGTTCGAGACCAGCCTGGCCAACATGGC
AAAACTCCATCTCTACCAAAAATACAAAAATTAGCTGGGTATGGTAGTGC
ACGCCTGTAATCCCAGCTACTTGGAATGCTGAAGCAGGAGAATTGCCTGA
ACCCANGAGGCGGAGGTTGCGGTGAGCCAAGATCACGCCACTGCACTCCA
ACCTGGGTGACAGAGCAAGGCTGTGTCTCAAA
SEQ ID NO: 368
ACCTGACTTCAAACTATACTACGAGGCTACAGTAATCAAAACAGCATGGT
ACTAGTACAAAAACAGACCAATGGAACAGAATAGAGATCTCAGAAATAAA
ACTGCACATCTACAACCATCTGATCTTCAACAAACCTGACAAAACGAGCA
ATGGGGAAAGGATTCCCTATTTAATAAATGGTGCTGGGAGAACTGGCT-A
GCCATGTGCAGAAAATTGAAACTGGACCCCTTCCTTACACCTTATACAAA
AATTAACTCAAGATGGATTAAAGACTTAAATGTAGAACCCAAAACGATAA
AAACCCTAGAAGAAAATCTAGGCAATATCATTAAGGACATAGACATGGGC
AAAAATTTCATGATGAAAACATCAAAAGCAATGGCAACAAAAGCAGAAAC
TGACAAATGGGCTTCTGCACAGCAAAAGAAACTATCGTCAGAGTGAACAG
ACAACCTACAGAATGGGAGACAGTTTTTGCAATCTATCCATCTGACAAAA
GTCTAATATCCAGAATCTACAAGGAATTTAA
SEQ ID NO: 369
CAAAAAACAAGAATTACCCGGGCTTGGTGGTGCATGTCTGTAGTCCTATC
TACTCAGGAGGCTGAGGCTGAAGGATCACTTGAGCCCAGGAGTTTGAGGC
TGCAGTGAGTGAGCCATGATCATGCCAGTGTACTCCAGCCTTGGCAGACT
GAGCAAAACTTGGTCCCTCGCAAAATGTTGAAGCCCAGTTTTCACTATTA
ACCTGTATTTCAGTTTCCCCATGCTAACTTTGAAACACTGGGGCTGGCCT
GAGGGTATAAAGGCTTATTCAAACTCAGTAATTTAAACTTAAAATCCTAA
GGAACTTCAAAAAGTGTAATCTAGTCCAAATGGGGCATCAATTCTAAAGC
ATTTGCTTGTTTGAGCAGATTTTCTGTGTCTGAGGTATATAGATAACTTA
TCTTTTTATGACTAAATCCAAGTCCTTAGTTCCTGTTGGAATTCAAAATC
ATATTTAAAAATTGATGCTTTGTTCTATAATTAATGCTTTGATTGTATAA
ATAATAAGTATTCTTCCAAATCCCTTTTTACAGATGATGATTCTGATACC
GAGACGTCAAATGACTTGCCAAAATTTGCAGATGGAATCAAGGCCNGAAA
CAGAAATCAGAACTACCTGGNTCCCAGTCCTGTNCTTAAAATTCTAACTC
GAC
nt: 595
SEQ ID NO: 370
GAGGGTGTAGAAGAGAAGAAGAAGGAGGTTCCTGCTGTGCCANAAACCCT
TAAGAAAAAGCGAAGGAATTTCGCAGAGCTGAAGATCAAGCGCCTGAGAA
AGAAGTTTGCCCAAAAGATGCTTCGAAAGGCAAGGAGGAAGCTTATCTAT
GAAAAANCAAAGCACTATCACAAGGAATATAGGCAGATGTACAAANCTGA
AATTCGAATGGCGAGGATGGCAAGAAAAGCTGGCAACTTCTATGTACCTG
CAGAACCCAAATTGGCGTTTGTCATCAGAATCAGAGGTATCAATGGAGTG
AGCCCAAAGGTTCGAAAGGTGTTGCAGCTTCTTCGCCTTCGTCAAATCTT
CAATGGAACCTTTGTGAAGCTCAACAAGGCTTCGATTAACATGCTGAGGA
TTGTAGAGCCATATATTGCATGGGGGTACCCCAATCTGAAGTCAGTAAAT
GAACTAATCTACAAGCGTGGTTATGGCAAAATCAATAAGAAGCGAATTGC
TTTGACAGATAACGCTTTGATTGCTCGATCTCTTGGTAAATACNGCATCA
TCTGCATGGAGGATTTGATTCATGAGATCTATACTGTTGGAAAAC
nt: 651
SEQ ID NO: 371
CATTTCCAGAGTTTATGTGAATTGAATTGAACTATGGTTTTATGTTACTG
TCAGTAGAATGAAGTACGAATATTTGAAAAATACACCTTCAACTTCAAAG
TGATTCTTGACAAAAATTATAAGGAATCATTTTGGACACATTTTCTGGTA
GAGCCTTGTAAAAATTAAAACCAAGTGTTGTTTTCAAGAAGAACTGTAAT
ACATAATCAGGAATTTGAGTAGGGAGATTATTTTGTTATTTAAAATTAAA
GTGGCTGTGTAGTTTTAACTTTAGTATTGCAGGTAGAGTAAGCTTACATG
ATAACAAAAATCTTGGTCTTAGTGACTTAATGATTCTGATATTTATTGAT
TGATTGGTTATCATTCCAAATATTTTAAAAGATAATAGCTGGCTGGGTGC
GGTGGCTCATGCCTGTAATCCCAGCACTTTGGGAGGCCAGGACGGGCGGA
TCACGAGGTCAGGAGATCAAGACCATCCTGGCTAACACGGTGAAACCCCG
TCTCTACTAAAAATCAAAAAATTAGCCGGGTGTAGTGGCGGGCACCTGTA
GTCCCAGCTACTCAGGAGGCTGAGGCAGGAGAATGGCATGAACCTGGGAG
GCGGAGCTTGCAGTGAGCTGAAATCGTGCCACTGCCTCCACCTGGCGACA
A
SEQ ID NO: 372
GTGAGGTGGGGACTTCATTCATTGTCCTATTTCTATCTCCACTTTGTGCC
TGGAGAGCTTTCAGGGGAGGTGGAGGAGGAGGGTCTGCCAAGCTACTGCA
ACATCTGTCACCCACTATACCCAGTTACTTGGGGGAGGACAGACACTGTG
GTGTCATTAAAGTTGTTTGAACCAAAGTGGCGGCTGCATCTTTGTCCCGA
TGCTAGCCGTGCCGGTCTCCCATCATCCGCTCGCCCTCCTTTNCCCTGGG
CTGCGCCCACTTGTCTTCCTGGATATTTGGGGGTGACTCGCCATGCTTGG
CACCCTCTGCTTCCTGGTGCTGCTCTGACTCGAAGACGGGACAGTCCCTG
GTGCACATCCAGGGAAGAGGAGTGTCGGTAGTTCTTGCAGTAGGCACTTT
ATCAGGACCTGACCTGTTGCTGGGTGATTTTAGTCTCTACAAACAGAAAG
CGTTTCAAAGCGTCAGCTGTGGGAGCAGAGTGACCCTTTGCTGATGCTGG
GGGGAGGGGATCTAAATCCTCATTTATCTCT
SEQ ID NO: 373
GGCGCCTGCTGGAGGAGGAGAGAGCTCTGCTGGCATGAGCCACAGTTTCT
TGACTGGAGGCCATCAACCCTCTTGGTTGAGGCCTTGTTCTGAGCCCTGA
CATGTGCTTGGGCACTGGTGGGCCTGGGCTTCTGAGGTGGCCTCCTGCCC
TGATCAGGGACCCTCCCCGCTTTCCTGGGCCTCTCAGTTGAACAAAGCAG
CAAAACAAAGGCAGTTTTATATGAAAGATTANAAGCCTGGAATAATCAGG
CTTTTTAAATGATGTAATTCCCACTGTAATAGCATAGGGATTTTGGAAGC
AGCTGCTGGTGGCTTGGGACATCAGTGGGGCCAAGGGTTCTCTGTCCCTG
GTTCAACTGTGATTTGGCTTTCCCGTGTCTTTCCTGGTGATGCCTTGTTT
GGGGTTCTGTGGGTTTGGGTGGGAAGAGGGCCATCTGCCTGAATGTAACC
TGCTAGCTCTCCGAAGCCCTGCGGGCCTGCTTGTGTGAACCGTGTGGACA
GTGGTGGCCGCGCTGTGCCTGCTCGTGTTGCCTACATGTCCCTGGCTGTT
GAGGCGCTGCTTTAACCTGCACCCCTNCCTTG-CTCATANATGCTCCTTT
TGA
nt: 230
SEQ ID NO: 374
TTTGAGACCAGCCTAGCCAACATGGTGAAACCCCATCTCTACTAAAAATA
CAAAAATTAGCCGGGCGTGGCGGCACATGCCTATAATCCCACTTACTTGG
GAGGCTGANGTAGGAGAATCGCTTGAACCCANANAGGCAGAGTTTGCAGT
GAGCCGAGATTGTGCCATTGCACTCCAGCCTGGGCGACAGAGCGAGACTC
CATCTAAAANAAAATAAATGAATAAAATAA
SEQ ID NO: 375
NNCAGATTTTTTTTTTTTTTTCAGNGTTAGACCATCTTTCAATTCCTGGA
ACAAACTTAACTTTCCATGATATGTATTTTTTATACATTGCTGGATTTTA
TTTGCTAATATTTTACTTAGGATTTAATTTTCTAAGTNGACCTATAATTN
TCCTGTATAAAATTGCATTTGTCACATTTTAGTATCAAGGTTGTCCTANC
NCCATGAAATGGATTTANAATGGTTTATGTAANATAAAGTACATTTCTTC
TAAAGGTTTGNGTGGATTAACTTTCAAATCTGCCANAGNGNGTTTTTTTC
CTTTTTTTTTTTTTTTCATTTNAAGGGAGNGCAAGTANCTTTTCAAATNC
TGATTTAATTTTTAAAATATTTNCAAGTNTNTTTANAGTTTTTATTTNTT
NTNGAANGTTAACATTTTTATANAAAANGGTNTTATCTTTTTAAATTCTT
TGACATCAGTTTCTTCANAATTCCTTCTTTTAA
SEQ ID NO: 376
GTCATATCTCTTCCCAGGGAAAGCAGGAGCCCTTCTGGAGCCCTTCAGCA
GGGTCAGGGCCCCTCGTCTTCCCCTCCTTTCCCAGAGCCATCTTCCCAGT
CCACCATCCCCATCGTGGGCATTGTTGCTGGCCTGGCTGTCCTAGCAGTT
GTGGTCATCGGAGCTGTGGTCGCTACTGTGATGTGTAGGAGGAAGAGCTC
AGGTAGGGAAGGGGTGAGGGGTGGGGTCTGGGTTTTCTTGTCCCACTGGG
GGTTTCAAGCCCCAGGTAGAAGTGTTCCCTGCCTCATTACTGGGAAGCAG
CATCCACACAGGGGCTAACGCAGCCTGGGACCCTGTGTGCCAGCACTTAC
TCTTTTGTGCAGCACATGTGACAATGAAGGACGGATGTATCACCTTGATG
GTTGTGGTGTTGGGGTCCTGATTTCAGCATTCATGAGTCAGGGGAAGGTC
CCTGCTAAGGACAGACCTTAGGAGGGCAGTTGGTCCAGGACCCACACTTG
CTTTCCTCGTGTTTCCTGATCCTGCCTTGGGTCTGTAG
SEQ ID NO: 377
TGGCCATCCTTTTCCCCCCAAACACACCCCCTTAACCTATCTCTTGGGAC
TTAGCCCGACCCTCCCTCTCATTTCCCATTAAGTCTGAGAGGCAAGAGCT
AGGTTAGGCAAGGAGGTGGTTGGCCAGAGATGGGGAACAGCCAGGTGCCC
CAGTCCTCTGATTTTTCCTCCATCCTGCTTACCACCTCCCTGGGTACTTA
CAGCCTTCTCTTGGGAACAGCCGGGGCCAGGACTGGGTCACCTATGAGCT
GAATCAGCATCTCCTCCTGAGTCCCAGGGCCCCTGCAGTTCCCAGTCTCT
TCTGTCCTGCAGCCCTTGCCTCTTTCCCACAGGTTCCACTTTATATCCAC
CTTTTCCTTTTGTTCAATTTTTATTTTTATTTTTTTTATTATTAAATGAT
GTGGTCTATGGAAAAAAAAATAAAAATCTGACTTAGTTTT
nt: 513
SEQ ID NO: 378
GGAACCCAGTGTATTACCTGCTGGAACCAAGGAAACTAACAATGTAGGTT
ACTAGTGAATACCCCAATGGTTTCTCCAATTATGCCCATGCCACCAAAAC
AATAAAACAAAATTCTCTAACACTGCAAAGAGTGAGCCATGCCTGTTAAC
ACTGTAAAGAATGTAACATGTGGGGGACACACAGGGGCAGATGGGATGGT
TTAGTTTAGGATTTTATTAGTGCATGCCCTACCCTCTGGGGGAACGTCCC
ATCTGAGGTTTTCTTCTCGGTGGGGGGATTTAACTTCTGTCCTAGGGAAA
ACAGTGTCTGATGAGGAGTGTTTCCAACACAGGCTACATGAATTCCCCTA
TACCAGTGCGAAAGCAGCCAGGAGTCCCCGTTGGAAAAGAACAATGCCAC
TCTCTTTTATGTATCTTGGTTCTGCAACTCATTTGTTGTAAGTAGGGTTA
ATCGAGTATCAGGTTCACAGTATCCTGCCCTTATTATTTTATGATTCACT
GACTCAAGTTCCA
SEQ ID NO: 379
GAGAGTGAAAAAATTCTGGTACAAATTGGGAAATTAGTATATAACAACAT
AGTGTTAAATTCAATGGGAAAAGTTTAATAAGAGGATTTGGTATCAACTG
GCTGTCCAAAGATAAAAATGGACCGTCCTATCACATACAAAATTGTTTTT
TAGATAAAGATTTAAATACAGGCACTCCTTCATTTGCGTGGTGCACCTTG
AGGTGTTGCAGAAATGATGAGAGCTGAAACTGCAAAGCAATTTTAATACT
TTATCTGTTGGAAATCTTATAGTTTTCCTGTGACCGTTAAAATTTTCATT
AAACTATTAAAAACACCCATGACTGGTCACAAATGTATTGGGAAATGGAA
AAGAATTAATACACTAAAAATACAAAAAATAGAAAATATTTAAAATTATC
TAAAAATTTGAAACATTAGAAAAATTGAGAACTAGGCAGGGCGTGGTGGC
TCACATCTGTAATTTTAGCCCTTTGGGAGGCTGANGCAGGTGGATCACCT
GANGTCAGGAGTTCGAGACCAGCCTGCCAACGTGGGGAAACCCCGTCTCT
ACTGAAAATACAAAAATTANCCGGGCATGGTGGCACAAGCCTGTAATNCT
TGCTNACCAGGANGCTGAGGCAGGAGAATCACTTGAACCCANGANG
SEQ ID NO: 380
GTTTCACATGAGAAGGTAGTATTATGTACAGTGACCTTGTTTAAAGTGTC
NGTTTAATGTTACCACTAAGGCCCTGCCCCAGCTTTATCACCTGAGCACT
AACAAGTGCTGTGTGGAGTTCAGTCCATGCTGGTAACTNTTGAGTATTCA
GTGGGTCTTTTAACAATTACCACCGTGGAGGANANAGCAAGGAAGAGAAA
TGCTGTGATCTTTTNCTGTTTTTAATTAGNGAAAGAGGGATTANATTAAA
CAAATGTTACAGAGNTGTGACTNTGATCCCCCAGNGGTAAGCAATAATTG
TANAGACTGGATTTNANAAGCCCTGAGAGTTTATTTTCAACCTATNTATT
ATAGNNCAATCC
SEQ ID NO: 381
ACAAGGCTTGGGGGCTGGACTCCCTCTACTGCCTCTGGCCATACCCCCTC
CTGGAGATGGGGTCAAGGCACCAGGACTGA
nt: 435
SEQ ID NO: 382
TCGCTTGTAAAGCCTGAGACAGCTGCCTGTGTGGGACTGAGATGCAGGAT
TTCTTCACACCTCTCCTTTGTGACTTCAAGAGCCTCTGGCATCTCTTTCT
GCAAAGGCATCTGAATGTGTCTGCGTTCCTGTTAGCATAATGTGAGGAGG
TGGAGAGACAGCCCACCCCCGTGTCCACCGTGACCCCTGTCCCCACACTG
ACCTGTGTTCCCTCCCCGATCATCTTTCCTGTTCCAGAGAAGTGGGCTGG
ATGTCTCCATCTCTGTCTCAACTTCATGGTGCGCTGAGCTGCAACTTCTT
ACTTCCCTAATGAAGTTAAGAACCTGAATATAAATTTGTTTTCTCAAATA
TTTGCTATGAAGGGTTGATGGATTAATTAAATAAGTCAATTCCTGGAAGT
TGAGAGAGCAAATAAAGACCTGAGAACCTTCCAGA
SEQ ID NO: 383
NGATATAGTNCCGCATGGGAAAGATGANCAGGTATAACCNAGCNTNATAT
AGCAAGGACTAACCCCCCTGCCTTCTGCATAATGAATTAACTAGAAATAA
CTTNGCAAGGAGAGCCAAAGCTAAGACCCCNGAAACCAGACGAGCTACCT
AAGAACAGNTAAAAGAGCACACCCGTCTATGTAGCAAAATAGTGGGAAGA
TTTATAGGTAGAGGCGACAAACCTACCGAGCCTGGTGATAGCTGGTTGTC
CAAGATAGAATCTTAGTTCAACTTTAAATTNGCCCACAGAACCCTCTAAA
TCCCCTTGTAAATTTAACTGTTAGTCCAAAGAGGAACAGCTCTTTGGACA
CTAGGAAAAAACCTTGTAGAGAGAGTAAAAAATTTAACACCCATAGTAGG
CCTAAAAGCAGCCACCAATTAAGAAAGCGTTCAAGCTCAACACCCACTAC
CTAAAAAATCCCAAACATATAACTGAACTCCTNACACCCAATTGGACCAA
TCTATCACCCTATAGAAGAACTAATGTTAGTATAAGTAACATGAAAACAT
TCTCCTCCGCATAAGCCTGCN
nt: 689
SEQ ID NO: 384
GGGAGGCGGAGGCTGCAGTGAGCTGAGATCGTGCCACTTCATTCCAGCCT
GGGCAACAAAGCGAAACTCTGTCTCAAAAAAAAAAAAAAAAAAAATTTGT
TGACTGTTGTAATTTAAAGCTTGTCATTTTTTATTTAGTAATAACACTCA
TTAGTGTAGTATCTATGATGAACCAGGTTCTGCACAAAGTACCTTATGTT
CATGGCCTCATATCGTCTTCTCCAAAACTCTGCAAGATAGGATTCATCAC
CACTTATAGGGAGAGATCTGAAAGTTTAAAATTGTACCCAAGGTCACACA
GCTGGTAAGTGCCAGAGCTGGGATTCCGTAGGGTGTTCANAGTGCCTCTC
CTGCCGTAGGCTTATCACAAAAAGTCAAAGTTTGGTCATAATAAAGCCTG
AAGTTTGGCAGGATTTAAAAATAGTCACCANACTTTTGAGTTGGAGCATC
CCACCTCACTGCTGTTCACCTTCTGTGGCAGGGAGAGTCATCATTTCCAT
TTCAGCTTGTGGAATATCTTGTCATTAACATTCTCATGCAAAAGCCATTT
TATGGTGCCCAATGAANATGGTTAAGCTACTGCCCCAAGCCTNTGGAAGC
CTTCCTAATTTTGGACTTGCACTATGCAAATTGNATAATATTTTCTCTAC
CCTAAGCCAAATATTTTCTTCACTTTTCATTCATTCTAC
SEQ ID NO: 385
CGCCGCCGCGCCGCCGTCGCTCTCCAACGCCAGCGCCGCCTCTCGCTCGC
CGAGCTCCAGCCGAAGGAGAAGGGGGGTAAGTAAGGAGGTCTCTGTACCA
TGGCTCGTACAAAGCAGACTGCCCGCAAATCGACCGGTGGTAAAGCACCC
AGGAAGCAACTGGCTACAAAAGCCGCTCGCAAGAGTGCGCCCTCTACTGG
AGGGGTGAAGAAACCTCATCGTTACAGGCCTGGTACTGTGGCGCTCCGTG
AAATTAGACGTTATCAGAAGTCCACTGAACTTCTGATTCGCAAACTTCCC
TTCCAGCGTCTGGTGCGAGAAATTGCTCAGGACTTTAAAACAGATCTGCG
CTTCCAGAGCGCANCTATCGGTGCTTTGCAGGAGGCAAGTGAGGCCTATC
TGGTTGGCCTTTTTGAAGACACCAACCTGTGTGCTATCCATGCCAAACGT
GTAACAATTATGCCAAAAGACATCCAGCTAGCACGCCGCATACGTGGAGA
ACGTGCTTAAGAATCCACTATGATGGGAAACATTTCATTCTC
nt: 198
SEQ ID NO: 386
GCGCGTCGACTTTGTTTAGACATTGAATGACTTTGTTAAAGGCACAATTA
ATCACATTGGTTGTACTCTGNNGACAGCCTTCTTTAAAAAAAAAATAAAC
AATTTAAAACAAAAAAAAAAAAAAAAAAAAAAAAAAAAAAAAAAAAAAAA
AAAAAAAAAAAAAAAAAAAAAAAAAAAAAAAAAAAAAAANTTTTAACC
nt: 198
SEQ ID NO: 387
GCGCGTCGACTTTGTTTAGACATTGAATGACTTTGTTAAAGGCACAATTA
ATCACATTGGTTGTACTCTGNNGACAGCCTTCTTTAAAAAAAAAATAAAC
AATTTAAAACAAAAAAAAAAAAAAAAAAAAAAAAAAAAAAAAAAAAAAAA
AAAAAAAAAAAAAAAAAAAAAAAAAAAAAAAAAAAAAAANTTTTAACC
nt: 561
SEQ ID NO: 388
TGCATGCTTGTGGATTGGAAAAACTTTGGAGACTGATTACTTTTCATTAT
ATATGTGTCACAGTGAAACAGCTTTTATGTGTCATGTAAGATTACTGCTT
GCCTCTCTAAGGAAGGTCGTGACTGTTTAAATAGACGGGCAAGGTGGAAC
CTTTTGAAAGATGAGCTTTTGAATATAAGTTGTCTGCTAGATCATGGTTT
GTATTGAACTAACAAGGTTTGCAGATCTGCTGACTTATATAAAGCTTTTT
GATTCCTACTAAGCTTTAAGATTTAAAAAATGTTCAATGTTGAAATTTCT
GTGGGGCTCTATTTTTGCTTTGGCTTTCTGGTGAGAGAGTGAGGAAGCAT
TCTTTCCTTCACTAAGTTTGTCTTTCTTGTCTTCTGGATAGATTGATTTT
AAGAGACTAAGGGAATTTACAAACTAAAGATTTTAGTCATCTGGTGGAAA
AGGAGACTTTAAGATTGTTTAGGGCTGGGCGGGGTGACTCACATCTGTAA
TCCCAGCACTTTGGGAGGCCAAGGCAGGCAGAACACTTGAAGGAGTTCAA
GACCAGCGTGG
SEQ ID NO: 389
TTTGNCGGTNTTGGANNNNNANAANTTTCTTCCANNCNTNACNTNTTGGT
GGNCTAAATTAANATGGNTTTNGNGGGTTCNTTNCTNNNTNNNNCATGGG
ANANAATTNATTNTCNTNCNNNTTCCTTNNCCCTNAANCTACCTTCCCCC
NATTTTCTCCCCTNTTCNTNAATTANCATCCTCTCCNCNTANNTCNANAC
NTTAATGGCAANACTATCTAATANCNANNATAANANCTCCTGTNNNCCAC
ATNTCTTATTNNNCGCNNCANGTTNCANNCCCNCAGAGTNAACTCATCCT
CNNCNNAANTTCATATCGTGNNCTNTNNNCNNTNGCGCGANATATTAANN
ANACCNGTANNTNNNANACANNANNTNNGNAANAANCCTTCTNANNTTTT
AGCNTCNNGCNNTAACNNNNNTCTTNGTGNNNNCNCAGCTTTCNCNNCAT
NATNCTNCNNCGAANTNTCANNCNTCTCCNCTTNAATGNNTTCCCATGNA
TTAANTNCCTCGNNNANAGCACTATCGTNNNNGAGNNNATTATNGNCNNT
TTACNTCATGTGGTCCANTNNCGTTNGNCGCNNNNAATNTTCGTNNNNCN
N
SEQ ID NO: 390
GGATTTTAGAGGAAGGCGCTNGGTTACATTGGAGAACTGGAGTGGTCTGG
AGTTCCACGGTGTAGTGGACCAGAGGCCACCTCTCCTGGGCTTCTCAGTG
TCTCGCCGGCGGGGTTCGGCCTGAGCTGGATTGACATAGCCCTTGGCGGA
TTTAAACAACCTAAACATTAAGCAGTACAGCTGCCTCAAACCTTTGGGAT
TTTCAGAATGACTGACACTGCCGAAGCTGTTCCAAAGTTTGAAGAGATGT
TTGCTAGTAGATTCACAGAAAATGACAAGGAGTATCAGGAATACCTGAAA
CGCCCTCCTGAGTCTCCTCCAATTGTTGAGGAATGGAATAGCANAGCTGG
TGGGAACCAAAGAAACAGAGGCAATCGGTTGCAAGACAACAGACAGTTCA
GAGGCAGGGACAACAGATGGGGGTGGCCAAGTGACAATCGATCCAATCAG
TGGCATGGACGATCCTGGGGTAACAACTACCCGCAACACAGACAAGAACC
TTACTATCCCCAGCAATATGGACATTATGGTTACAACCAGCGGCCTCCTT
ACGGTTACTACTGATAGAAATGTTGGCAGCTTTTAGTAAAAGCATTTACT
CTGTTACCATGAGAAA
SEQ ID NO: 391
NGACTGGCTCCCGAAAAGAAGGGTGGCGAGAANAAAAAGGGCCGTTCTGC
CATGGACGAAGTGGTAACCCGCGAATACACCATCAACATTNACAAGCGCA
TCCATGGAGTGGGCTTCAAGAANCGTGCACCTCGGGCACTCAAAGAGATT
CGGAAATTTGCCATGAAGGAGATGGGAACTCCATATGTGCGCATTGACAC
CAGGCTCAACAAANCTGTCTGGGCCAAAGGAATAAGGAATGTGCCATACC
GAATCCGTGTGCGGCTGTCCANAAAACGTAATGAGGATGAAGATTCACCA
AATAAGCTNTATACTTTGGTTACCTATGTACCTGTTACCACTTTCAAAAA
TCTACAGACAGTCAATGTGGATGANAACNAATCGCTGATCGTCAGATCAA
ANAAANT
nt: 503
SEQ ID NO: 392
CAGCACTGCCAGTGGAGATGGGCGTCACTACTGCTACCCTCATTTCACCT
GCGCTGTGGACACTGAGAACATCCGCCGTGTGTTCAACGACTGCCGTGAC
ATCATTCAGCGCATGCACCTTCGTCAGTACGAGCTGCTCTAAGAAGGGAA
CCCCCAAATTTAATTAAAGCCTTAAGCACAATTAATTAAAAGTGAAACGT
AATTGTACAAGCAGTTAATCACCCACCATAGGGCATGATTAACAAAGCAA
CCTTTCCCTTCCCCCGAGTGATTTTGCGAAACCCCCTTTTCCCTTCAGCT
TGCTTAGATGTTCCAAATTTAGAAAGCTTAAGGCGGCCTACAGAAAAAGG
AAAAAAGGCCACAAAAGTTCCCTCTCACTTTCAGTAAAAATAAATAAAAC
AGCAGCAGCAAACAAATAAAATGAAATAAAAGAAACAAATGAAATAAATA
TTGTGTTGTGCAGCATTAAAAAAAATCAAAATAAAAATTAAATGTGAGCA
AAG
nt: 587
SEQ ID NO: 393
TGAAAAATAAAGTTTTTATGTATATTCTACATATGTATATGTTGGTAGAA
AGCAAAAACGCTAGGTAAAAATAAATGTAATACAATTTTAGCTATGAACC
AAAAAACCATTTGTGGTGTGGATGCAAGAAAGTCTGGATGGGTGCAGAGT
TCTCCATGTTTCACTTCTGACATTTGAAAATACGCAGTTTGCATTTGATA
CGTCAAATGTTATTTTTAAGAAAACCAATAAAATCATTAAAACCGAAAAG
GCAGTTTTGCTTGTTTTTACCTTAGTTGGAGTTATCTGCAATTGCCGTAT
TAGTGTTTTAAGGAACTTGTAAGTAAGCTCCTTAGTCCCCTTTAGAGCTA
CGAAACATGTCAATTTTACTTTTCTCCAGCTTTTTGGAATCTTATCTAAA
TTACCATGTAGAGTTCTGCATAGCTTCAAATTCTCTTAGCCAATGTGGTC
TGTAAGTGTCTATCGATGAATTTCACCGTTAATTGCCGTAGTATACTGTC
CTGTACCGGATGTGAAGAGGAGCAACTCTGCACAGTGCACTGGTTGCTCC
CATGGTAGGAANGAATGGCTTATCAATGGTCGGATTT
nt: 650
SEQ ID NO: 394
GGAGGATGGAGCAGTGAGCGGGTCTGGGCGGCTGCTGGCAGCGCCATGGA
GACGGTACAGCTGAGGAACCCGCCGCGCCGGCAGCTGAAAAAGTTGGATG
AAGATAGTTTAACCAAACAACCAGAAGAAGTATTTGATGTCTTAGAGAAA
CTTGGAGAAGGGTGAGTGTAAAGAAACTATAGGTAGGTCATTGGGTCCCA
GTCTTTTTCCTGCCCCAGAAGAAGCAGAAGGATATGAACCTTTCAGCATT
GTTCTAGGTGGGGTGGAAGGTAAATTTACAGCTTGTGATGTCCTTCTTCG
CTTTACTCCAATCCCTATTATAGACAGATTTAGTGATTCCTGGTCTTTTT
AACACGAAGAATATCTATTGTTTTCTCTTTTGTAGGATCTGTATGATTTT
ATCTACTTAACAGATAGCACTAATTAGATTAAAATTCTATAAGAAACTTT
TTAATTTGCTGTTCATAATTTCTGATTGGTATGCAATAACTGTTTCAATG
AAAATCAATGTAATTTAGTATTTTAATATTTGCACCTTTGTGAAATATAG
TAAATAAATTAAGCACTATCACCACCTTCACAGCTACTTAGGAGATCCAC
AATCCTGGGTTGGGAGCCAGTGGATTTCCTGAAACACAGATTTGTTAATG
nt: 502
SEQ ID NO: 395
CTCAAGTGAATCCTGGCTTCTTGGAAGCGCTTGCCTAGACGAGACACAGT
GCATAAAAACAACTTTTGGGGGACAGGTATGTTTTCTTGCAGCTGCGGTT
GTAAGGTCTTGGCAAGACAAGCAGTGTGGCCAGAATTTTGAACTTCTGAT
GAATGTGTAATGCAAAGGACCTTGTACATTTTTTTGTTTCAAGGTCCTCA
AAATGAGCACATGAAGAGGTTGCTGTGAAACTTTAAGTGGCCCTACTGCG
CAGAAGCATTCAGATGTCACTTGATGATCTGTAAGGGAACTTGCTGATTT
GGGAATGTGCTTAGGGAACACACATTCCTTTTGACAGGGTCTGTCACTGG
GTGGGTGATGAATTATACAGATGACATGTGCTTTTTTTTCTTTTTTCAAC
CTCAATGGTATTCCTACAGGAAATGGATAACCATTTTAACTGTATTTTTT
GCAGCCCGTACCTTCTTGGGAATACAATTGTCTAACTTTTTATTTTTGGT
CT
nt: 648
SEQ ID NO: 396
CCACAATAATAAGAGAAAAACAGGAGCAAAAGGATATACAAAACCACCAG
AAAACAAATAACAAAGTGACAGGAGTAAGTCCTTAACTGGCAATAATAAC
CATGAATCTAAATGGATTCCATTTCCCACTTAAAAGATAAAGACATGCTG
AATGGATAAAAAGCTGTCACCCAGTTATATGCTGCCTACAACAAACTCAC
TTCACCTGTAAACATACATATGGATGGAAAGAGAAGGCATGGGAAAAGAT
ACTCTACTCAAATGAAAACAAAAACCAAACAAAGGTGGCTATTCTTATAT
GAGATAATACAGACATTAAATCAAAAACTGGAAACAAACACAAAGTCATT
GTATAATGATGAATTCAATTATATCATGATGAATTCAATTATATCCTCCT
TCCTGATCAATTCAGAAAGGAGGATATAATCTTTTTAAATATATATACAC
CCAACACCAGAGCATATAAATATGTAAAGGAAGATAAAGGGAGTCCTGTG
ATCAAGAATAAATATAACAATTATAAATATTTTATCTAAAGTGATAGATA
GACTGTAATACAATAATAGGGTGGTGACATTAACACCCCCTCTCACATTG
GACTGATCATCTAGAAGGGAGAAAAAGCTTTATGATTGGAAAAGCCAT
SEQ ID NO: 397
ATTGTGTTGGCCACCCGGGAATTCGCGGCCGCGTCGACCTACGCACACGA
GAACATGCCTCTCGCAAAGGATCTCCTTCATCCCTCTCCAGAAGAGGAGA
AGAGGAAACACAAGAAGAAACGCCTGGTGCAGAGCCCCAATTCCTACTTC
ATGGATGTGAAATGCCCAGGTGAGGAGACGGCTTGCTGTAGTGGGGAAAG
CACTGGACCTCAACAGTTGGAAAATGTTGTAGTGTTAGCTGTCTCGTATC
CTTGAAGCTGTGCAGCAGCTTCAGTTTCTTCGCCTGTGGAAAATATTTTC
CCTGATACTCTTAAAATTTGAATGTATGAGACTGGCAAAGTTTTGCATCT
TAGGAGGAGTGATTCATTTCACCGTGATCTCTCATCACATTTCACATACA
ACCCCTACGTTTTTTTGTGTTGGGAAACAATGTAATGGATGATGAGTTGG
GCATAAGTGCAGGAAAGACGGGTGTAATAGAGGAAAAAAATGTTATCTGC
TTTTCTTTCAGGATGCTATAAAATCACCACGGTCTTTAGCCATGCACAAA
CGGTAGTTTTGTGTGTTGGCTGCTCCACTGTCCTCTGCCAGCCTACAGGA
GGAAAAGCAAGGCTTACAGAAGGATGTTCCTTCAGGAGGAAGCAGCACTA
AAAGCACTCTGAGTCAANATGAGTGGGAAACCATCTCAATAAACACATTT
TGGAT
nt: 622
SEQ ID NO: 398
CTTTTCCTCCCGCTGTCCCCCACGGGAGGGGACTGCTCTCCCCCGCTGCA
TCCTTTCTGTGAGGTACCTTACCCACCTCAGCACCTGAGAGGGTGAAATA
GAATTCTAACCTCGACATTCGGGAAGTGTTTTTGAGAAGTCTCGGTCGGT
AAGGGAAGTCTTCCAAGTCCGTGCAGCACTAACGTATTGGCACCTGCCTC
CTCTTCGGCCACCCCCCAGATGAGGCAGCTGTGACTGTGTCAAGGGAAGC
CACGACTCTGACCATAGTCTTCTCTCAGCTTCCACTGCCGTCTCCACAGG
AAACCCAGAAGTTCTGTGAACAAGTCCATGCTGCCATCAAGGCATTTATT
GCAGTGTACTATTTGCTTCCAAAGGATCAGGCCCTGAGAACAATGACCTT
ATTTCCTACAACAGTGTCTGGGTTGCGTGCCAGCAGATGCCTCAGATACC
AAGAGATAACAAAGCTGCAGCTCTTTTGATGCTGACCAAGAATGTGGATT
TTGTGAAGGATGCACATGAAGAAATGGAGCAGGCTGTGGAAGAATGTGAC
CCTTACTCTGGCCTCTTGAATGATACTGAGGAGAACAACTCTGACANCCA
CAATCATGAGGATGATGTGTTG
nt: 155
SEQ ID NO: 399
CGCCACTTATCCAGTGAACCACTATCACGAAAAAAACTCTACCTCTCTAT
ACTAATCTCCCTACAAATCTCCTTAATTATAACATTCACAGCCACAGAAC
TAATCATATTAAAAAAAAAAAAAAAAAAAAAAAAAAAAAAAAAAAAAAAA
AAAAA
SEQ ID NO: 400
CATTGTGTTGGCNCCCGGGAATTCGCGGCCGCGTCGACTTTTTGTGTTGT
TTGGAGCAGAAATACTAAAGAAGATTCCGGGCCGAGTATCCACAGAAGTG
GACGCAAGGCTCTCCTTTGATAAAGATGCGATGGTGGCCAGAGCCAGGCG
GCTCATCGAGCTCTACAAGGAAGCTGGGATCAGCAAGGACCGAATTCTTA
TAAAGCTGTCATCAACCTGGGAAGGAATTCAGGCTGGAAAGGAGCTCGAG
GAGCAGCACGGCATCCACTGCAACATGACGTTACTCTTCTCCTTCGCCCA
GGCTGTGGCCTGTGCCGAGGCGGGTGTGACCCTCATCTCCCCATTTGTTG
GGCGCATCCTTGATTGGCATGTGGCAAACACCGACAAGAAATCCTATGAG
CCCCTGGAAGACCCTGGGGTAAAGAGTGTCACTAAAATCTACAACTACTA
CAAGAAGTTTAGCTACAAAACCATTGTCATGGGCGCCTCCTTCCGCAACA
CGGGCGAGATCAAAGCACTGGCCGGCTGTGACTTCCTCACCATCTCACCC
AAGCTCCTGGGAGAGCTGCTGCAGGACAACGCCAAGCTGGTGCCTGTGCT
CTCAGCCAAGGCGGCCCAAGCCAGTGACCTGGAAAAAATCCACCTGGATG
AGAAGTCTTTCCGTTGGTTGCACAACGAGGACCAGATGGCTGTGGAGAAG
nt: 479
SEQ ID NO: 401
CGTGGCAGCCATCTCCTTCTCGGCATCATGGCCGCCCTCAGACCCCTTGT
GAAGCCCAAGATCGTCAAAAAGAGAACCAAGAAGTTCATCCGGCACCAGT
CAGACCGATATGTCAAAATTAAGCGTAACTGGCGGAAACCCAGAGGCATT
GACAACAGGGTTCGTAGAAGATTCAAGGGCCAGATCTTGATGCCCAACAT
TGGTTATGGAAGCAACAAAAAAACAAAGCACATGCTGCCCAGTGGCTTCC
GGAAGTTCCTGGTCCACAACGTCAAGGAGCTGGAAGTGCTGCTGATGTGC
AACAAATCTTACTGTGCCGAGATCGCTCACAATGTTTCCTCCAAGAACCG
CAAAGCCATCGTGGAAAGAGCTGCCCAACTGGCCATCAGAGTCACCAACC
CCAATGCCAGGCTGCGCAGTGAAGAAAATGAGTAGGCAGCTCATGTGCAC
GTTTTCTGTTTAAATAAATGTAAAAACTG
nt: 628
SEQ ID NO: 402
CTTTGATTACCTTTGAGTATTAGGTTGAAAGCTTCTCTGTGCTTGATTGA
ACATTGTGATGATGTTGATTGGGTCATGTCAGATTTAGACAGTGTTGTGT
TTAAGATAAATGTTTAATGGCTCTTAGCAGTGTTCATGCCTCCCCTTTTC
CCCTGATACTTTAAAAACAGAATATACAGAAAAGGGGAGTTGGGTGAAGA
ATCACCATATTCTCATTACCAGAGTAGTGTCTACCAGCTGTTTTCACATT
TTTCTGTTTCCTTCTGTCCTTGGAATCCTTTTTTTAGATCCTTGTAATAC
TAGTAAAGATATTCCACTCTGTGTTGTAAGCATTTTTCCATTTTGCTCCA
TGGTCTTCATAATGCCCTGTGGTCCTTTATTAAGGGGATGCACCATGTAG
AGGTGAAAGGCTTTCCTTGACTTGGCCACCATTTCTGTATTTTCCTTAGA
GGAGGAGGTTTCCAACATTTCTTTTTTAGAGACAGAGTCTCGTTCTGACA
CGCAGGCAGGAGTGCAGTGGCATGATAACAGCTCACTGCAGCCTCGAACT
CCTGGGCTCAAGTTATCCTCCCACCTCAGCTTCCTGAGTAGCTAGGACTG
CAGGTGCCTGCCACCACACCCAGCTAAT
nt: 494
SEQ ID NO: 403
CAGCCCTCCGTCACCTCTTCACCGCACCCTCGGACTGCCCCAAGGCCCCC
GCCGCCGCCTCCAGCGCCGCGCAGCCACCGCCGCCGCCGCCGCCTCTCCT
TAGTCGCCGCCATGACGACCGCGTCCACCTCGCAGGTGCGCCAGAACTAC
CACCAGGACTCAGAGGCCGCCATCAACCGCCAGATCAACCTGGAGCTCTA
CGCCTCCTACGTTTACCTGTCCATGTCTTACTACTTTGACCGCGATGATG
TGGCTTTGAAGAACTTTGCCAAATACTTTCTTCACCAATCTCATGAGGAG
AGGGGAACATGCTGAGAAACTGATGAAGCTGCAGAACCAACGAGGGTGGC
CGAATCTTCCTTCAGGATATCAAGAAACCAGACTGTGATGACTGGGAGAG
CGGGCTGAATGCAATGGAGTGTGCATTACATTTGGAAAAAAATGTGAATC
AGTCACTACTGGAACTGCACAAACTGGCCACTGACAAAAATGAC
nt: 599
SEQ ID NO: 404
GGGAGACAAGCCCAGCCTTTCGGCGAGNATACGTCTAACCCTGTGCAACA
GCCACTACATTACTTCAAACTGAGATCCTTCCTTTTGAGGGAGCAAGTCC
TTCCCTTTCATTTTTTCCAGTCTTCCTCCCTGTGTATTCATTCTCATGAT
TATTATTTTAGTGGGGGCGGGGTGGGAAAGATTACTTTTTCTTTATGTGT
TTGACGGGAAACAAAACTAGGTAAAATCTACAGTACACCACAAGGGTCAC
AATACTGTTGTGCGCACATCGCGGTAGGGCGTGGAAAGGGGCAGGCCANA
GCTACCCGCAGAGTTCTCAGAATCATGCTGAGAGAGCTGGAGGCACCCAT
GCCATCTCAACCTCTTCCCCGCCCGTTTTACAAAGGGGGAGGCTAAAGCC
CAGAGACAGCTTGATCAAAGGCACACAGCAAGTCAGGGTTGGAGCAGTAG
CTGGAGGGACCTTGTCTCCCAGCTCAGGGCTCTTTCCTCCACACCATTCA
GGTCTTTCTTTCCGAGGCCCCTGTCTCAGGGTGAGGTGCTTGAGTCTCCA
ACGGCAAGGGAACAAGTACTTCTTGATACCTGGGATACTGTGCCCAGAG
SEQ ID NO: 405
GGGAGACAAGCCCAGCCTTTCGGCGAGATACGTCTAACCCTGTGCAACAG
CCACTACATTACTTCAAACTGAGATCCTTCCTTTTGAGGGAGCAAGTCCT
TCCCTTTCATTTTTTCCAGTCTTCCTCCCTGTGTATTCATTCTCATGATT
ATTATTTTAGTGGGGGCGGGGTGGGAAAGATTACTTTTTCTTTATGTGTT
TGACGGGAAACAAAACTAGGTAAAATCTACAGTACACCACAAGGGTCACA
ATACTGTTGTGCGCACATCGCGGTAGGGCGTGGAAAGGGGCAGGCCAGAG
CTACCCGCAGAGTTCTCAGAATCATGCTGAGAGAGCTGGAGGCACCCATG
CCATCTCAACCTCTTCCCCGCCCGTTTTACAAAGGGGGAGGCTAAAGCCC
AGAGACAGCTTGATCAAAGGCACACAGCAAGTCAGGGTTGGAGCAGTAGC
TGGAGGGACCTTGTCTCCCAGCTCAGGGCTCTTTCCTCCACACCATTCAG
GTCTTTCTTTCCGAGGCCCCTGTCTCAGGGTGAGGTGCTTGAGTCTCCAA
CGGCAAGGGAACAAGTACTTCTTGATACCTGGGATACTGTGCCCAGAGCC
TCGAGGAGGT
SEQ ID NO: 406
GTTTAAATTTGACAAACTAAAGCTNATNACTGCTATAAGAGTAATAACTG
CTCATTTTCCATAACTCATTCTTAAAGTTTTAGTAATGTAAAAGTTATTT
TTTTGCAGTAAGTTATAATGATAGAAGCTTACATGTTTTTTCATGCCTCA
TCTGTTTCCCCTTAAAACTATAATTATCAGTAAAGTCCTGTGGTATTTTT
CAATTTGTAAGAAACTAGGCTATATATACATTGGGAAAAACAGCCTTCAT
TTGTCAATGCACTAGTGTTCCAAAGGTTTCTGGTAATTGTGTGCTATTGC
TTTTTGTTGACTTGCAAAAAAAAAAAAAAAAAAATTACTATGACTTGNGG
TAGCCCTGCAACCTTCGGAAGTGCTTAGCCCAGTCTGACCATACATTTAT
ATTTANAATGCTTAGGTAAATAAATAATATGCCTAAACCCAATGCTATAA
GATACTATATAATATCTCATAATTTTAAAAATCACTGTTTTGTATAATAA
TAAAACAAGGCAGGCAAGCTGTTCTACAATGACTGTTGGTAAGGGTGCTG
AGGAAGAAAAACAAACAATCTTGATTCAGGGATAGTGAATAGACAAAAAA
TGTCCTAATCAATGAAGCTGTGTGATGATTCTGATTGACAGAGA
SEQ ID NO: 407
GTGCAAAGTGTTATATCCACTTTCAACAAAGAGAGAAGCTGAAAAGCTAA
CCCAATGTTAATTTTGGATCACACACATTCAGTGTAGACTTTAAGATTTT
ACTTCTGTTGGAGTAGCTATATTATTTCTAGTTAAAAAACTCTCTATATA
CATATTTATTTGTTTTTCTACTTGTTTAATATTTTTCTCTTCCAATTAGG
AACTCAATATGGAATAAAAAATATTTAAATGTATTTTACTCAAACGTGTG
TGTATATATGTTTGTGTGCATGATAAGGAGAGTGAGAGCAAGAGTAAGAG
AGAGAGAGCACGCATAGATGGAAGCACACATTTAATGTCTATGAAATGAG
AAAACATTAAGGCTAAGATATTTTTCCTTCTGAACTAGCAGATTGTATCA
ATGGCTGGTCACTTAAATTAATCAGTTTGTAAAGATATTTAAAAGGTATG
TCTACCTTCTTGCAATTAATTTGATTATGTTCTAATGGCATGGCAAGAGA
AATGAAAGAAGATAACTAAAAGTTAAAAGTCGTTGCATGTTTTTGTTGCA
GCATACCCTTCTTTCAGGCTACCGAATAACCTTGATTGACATTGGATTAG
TAGTAGAATACCTCATTGGTAGAGCATATCGCAGCANCTACACTAGAAAA
CAT
SEQ ID NO: 408
GTCTGGAACTCCAGACCTCAGGTGATACCCCTGCCTCAGCCTCCCAATGT
GCTGGGATTACAGCTGTGAAGCCACCGCGCCCGGCTGCTGTGATAGTTGA
GATGTAAACCAAAAATAAAATTCTAAGCCACCCAATCCGACTGAATGGAC
CCTTCCTGTTGAGCAAGGACATTCCAAAGTAAACTGAAAAGACCAGCTTA
GGCCATGATGGGAAGGGGAGGTGTCAACATGCCTCATTCTACCTTCCTCC
CTCTGGAATCCAGACACAACTGACCAGCATTAACATTAAAACAGAGATCT
TAAGCTGGGCACGGTGGCTCATGCCTGTAATCCCAGCACTTTGGGAGGCC
AAGGTGGGATCACCTGAGGTCGGAAGTTCAAGACCAGCCTGGCCGGTATG
GTGAAGCCATGTCTCTACTGAAAATGCAAAATTGGCCGGACATTGTGGTG
CA
SEQ ID NO: 409
TNCNTTTTTTTTCCCNCGGGAAAGCGCGCCATTGTGTTGGTCCCCGGGAA
TTCGCGGCCGCGTCGACGAGAAATGGCTTGAACCCAGTAGGCAGAGGTTG
TAGTGAGCCCAGAATNGGNCACCTGCACNTTTANCCNTGGGTGACAAAAN
TGAAAACTTTGTCTNAAAAAAAAAAAAAAAAAATTTTAANTNAAATNAAA
AANCCTTTNCNTTNTTTTTNAAANNGGGGGGGGNNTTTTTNGGGNTTNGN
NNTGGTAAAAANTNNNTTTTTTTTTTTTTAGGGGCCNANNCCCCNTTTTA
NAAAANCCNGNTTTTNAAAAAANTTTTTTNCCCNCNNTTNGGGGGGGGGG
NTTTTNANCNNTNTTNGGGGGGGNNCCCCTNTTANNACCNNCAAANTTTT
TANTTTTTTGNNNAANNNCCCCCTTTTTTNNTTTTTTTTGNGGGGGGGGG
GNNGCCCCCNNCCTTTNGGGGGGGGGGNTTNNGNAAAANNACTTTTNAAA
ANNAAGGGNNGGGGGNANATNNCCCCCCCNGGNTTTTTTTTTTAAAAANT
NAANNGGGGGGGGNNNCTNANTNGGGGCNCCCANNGGGGGNTTANAANNA
TTTTCTNCCCAAACCCCCNGNTTTTATNNCCCCCCCCCCCCNCNNNNGAA
NGGGNGGNCCNTTTTTTTTATTTTTNNGGNGGGNAAAAAANTTTNAAAAA
NNANNATNTTTTTTCCCCCCCCCCCCNCTTTTNGGNAAANCCNNGGGGGG
NTCCTTTTTNAAANNNNCCCCCAAAAAAAANTTTTTTTNTTNTNTTTTTC
TCTNGGGGNCCNNANTTNTANANTTTTNCNCCNAAAAAAAANGGGNCCCC
TTTTTTTNCNGGNNGGNNCCCAAAANNTTTTTTTTNAAAAAAAAAAAAAA
SEQ ID NO: 410
GTTCGTGACNTTCGGAGCTACCTGACAGAGCAGAGTCAACCAGGNTCTGC
CCAAAGAGAGTGTTAGGCCTGAGCTTGAGAGCCCTGGAGAGACGTGTGCA
CAAAATGTGACCTGAGGCCCTAGTCTAGCAAGAGGACATAGCACCCTCAT
CTGGGAATAGGGAAGGCACCTTGCAGAAAATATGAGCAATTTGATATTAA
CTAACATCTTCAATGTGCCATAGACCTTCCCACAAAGACTGTCCAATAAT
AAGAGATGCTTATCTATTTTA
nt: 412
SEQ ID NO: 411
GTCGACGCGGCCGCGGTCGCTGGAGNCGATCAACTCTAGGCTCCAACTCG
TTATGAAAAGTGGGAAGTACGTCCTGGGGTACAAGCAGACTCTGAAGATG
ATCAGACAAGGCAAAGCGAAATTGGTCATTCTCGCTAACAACTGCCCAGC
TTTGAGGAAATCTGAAATAGAGTACTATGCTATGTTGGCTAAAACTGGTG
TCCATCACTACAGTGGCAATAATATTGAACTGGGCACAGCATGCGGAAAA
TACTACAGAGTGTGCACACTGGCTATCATTGATCCAGGTGACTCTGACAT
CATTAGAAGCATGCCAGAACAGACTGGTGAAAAGTAAACCTTTTCACCTA
CAAAATTTCACCTGCAAACCTTAAACCTGCAAAATTTTCCTTTAATAAAA
TTTGCTTGTTTT
SEQ ID NO: 412
CCGCCAACATGGGCCGCGTTCGCACCAAAACCGTGAAGAAGGCGGCCCGG
GTCATCATAGAAAAGTACTACACGCGCCTGGGCAACGACTTCCACACGAA
CAAGCGCGTGTGCGAGGAGATCGCCATTATCCCCAGCAAAAAGCTCCGCA
ACAAGATAGCAGGTTATGTCACGCATCTGATGAAGCGAATTCAGAGAGGC
CCAGTAAGAGGTATCTCCATCAAGCTGCAGGAGGAGGAGAGAGAAAGGAG
AGACAATTATGTTCCTGAGGTCTCAGCCTTGGATCAGGAGATTATTGAAG
TAGATCCTGACACTAAGGAAATGCTGAAGCTTTTGGACTTCGGCAGTCTG
TCCAACCTTCAGGTCACTCAGCCTACAGTTGGGATGAATTTCAAAACGCC
TCGGGGACCTGTTTGAATTTTTTCTGTAGTGCTGTATTATTTTCAATAAA
TCTGGGACAA
SEQ ID NO: 413
CAGAGGTGGGAGGATTGCTTCAGTTCAAGAGTTTGAGACCAGCCTGGGTA
ACATGGCGAAACCCTGTCTTTACAAAAAATGCAAACCTTTGCCGCATGTG
TTGGGGTGCGCCTGTAGTCCCAGCTTCTCGGGAGGCTGAGGTGGGGGGAC
CACCTGAGCCATGGAGGTTGAGGCTGCAGTGAGCCGTGATACCACCACTG
TACTCTAGCCTGGGCCATAGAGTGAGACACCCTGCCTCAGAAATA
nt: 439
SEQ ID NO: 414
CCCATCCCCTCGACCGCTCGCGTCGCATTTGGCCGCCTCCCTACCGCTCC
AAGCCCAGCCCTCAGCCATGGCATGCCCCCTGGATCAGGCCATTGGCCTC
CTCGTGGCCATCTTCCACAAGTACTCCGGCAGGGAGGGTGACAAGCACAC
CCTGAGCAAGAAGGAGCTGAAGGAGCTGATCCAGAAGGAGCTCACCATTG
GCTCGAAGCTGCAGGATGCTGAAATTGCAAGGCTGATGGAAGACTTGGAC
CGGAACAAGGACCAGGAGGTGAACTTCCAGGAGTATGTCACCTTCCTGGG
GGCCTTGGCTTTGATCTACAATGAAGCCCTCAAGGGCTGAAAATAAATAG
GGAAGATGGAGACACCCTCTGGGGGTCCTCTCTGAGTCAAATCCAGTGGT
GGGTAATTGTACAATAAATTTTTTTTTGGTCAAATTTAA
nt: 526
SEQ ID NO: 415
CTGGAGACGACGTGCAGAAATGGCACCTCGAAAGGGGAAGGAAAAGAAGG
AAGAACAGGTCATCAGCCTCGGACCTCAGGTGGCTGAAGGAGAGAATGTA
TTTGGTGTCTGCCATATCTTTGCATCCTTCAATGACACTTTTGTCCATGT
CACTGATCTTTCTGGCAAGGAAACCATCTGCCGTGTGACTGGTGGGATGA
AGGTAAAGGCAGACCGAGATGAATCCTCACCATATGCTGCTATGTTGGCT
GCCCAGGATGTGGCCCAGAGGTGCAAGGAGCTGGGTATCACCGCCCTACA
CATCAAACTCCGGGCCACAGGAGGAAATAGGACCAAGACCCCTGGACCTG
GGGCCCAGTCGGCCCTCANAGCCCTTGCCCGCTCGGGTATGAAGATCGGG
CGGATTGAGGATGTCACCCCCATCCCCTCTGACAGCACTCGCAGGAAGGG
GGGTCGCCGTGGTCGCCGTCTGTGAACAAGATTCCTCAAAATATTTTCTG
TTAATAAATTGCCTTCATGTAAACTG
nt: 613
SEQ ID NO: 416
CTTAAGTATGCCCTGACAGGAGNATGAAGTAAAGAAGATTTGCATGCAGC
GGTTCATTAAAATCGATGGCAAGGTCCGAACTGATATAACCTACCCTGCT
GGATTCATGGATGTCATCAGCATTGACAAGACGGGAGAGAATTTCCGTCT
GATCTATGACACCAAGGGTCGCTTTGCTGTACATCGTATTACACCTGAGG
AGGCCAAGTACAAGTTGTGCAAAGTGAGAAAGATCTTTGTGGGCACAAAA
GGAATCCCTCATCTGGTGACTCATGATGCCCGCACCATCCGCTACCCCGA
TCCCCTCATCAAGGTGAATGATACCATTCAGATTGATTTAGAGACTGGCA
AGATTACTGATTTCATCAAGTTCGACACTGGTAACCTGTGTATGGTGACT
GGAGGTGCTAACCTAGGAAGAATTGGTGTGATCACCAACAGAGAGAGGCA
CCCTGGATCTTTTGACGTGGTTCACGTGAAAGATGCCAATGGCAACAGCT
TTGCCACTCGACTTTCCAACATTTTTGTTATTGGCAAGGGCAACAAACCA
TGGATTTCTCTTCCCCGAGGAAAGGGTATCCGCCTCACCATTGCTGAAGA
GAGAGACAAAAGA
SEQ ID NO: 417
GGAATTCGCGGCCGCGTCGACCTCTGCTCGAATTGACAGAAAAGGATTCT
GTGAAGAGTGATGAGATTTCCATCCATGCTGACTTTGAGAATACATGTTC
CCGAATTGTGGTCCCCAAAGCTGCCATTGTGGCCCGCCACACTTACCTTG
CCAATGGCCAGACCAAGGTGCTGACTCAGAAGTTGTCATCAGTCAGAGGC
AATCATATTATCTCAGGGACATGCGCATCATGGCGTGGCAAGAGCCTTCG
GGTTCAGAAGATCAGGCCTTCTATCCTGGGCTGCAACATCCTTCGAGTTG
AATATTCCTTACTGATCTATGTTAGCGTTCCTGGATCCAAGAAGGTCATC
CTTGACCTGCCCCTGGTAATTGGCAGCAGATCAGGTCTAAGCAGCAGAAC
ATCCAGCATGGCCAGCCGAACCAGCTCTGAGATGAGTTGGGTAGATCTGA
ACATCCCTGATACCCCAGAAGCTCCTCCCTGCTATATGGATGTCATTCCT
GAAGATCACCGATTGGAGAGCCCAACCACTCCTCTGCTAGATGACATGGA
TGGCTCTCAAGACAGCCCTATCTTTATGTATGCCCCTGAGTTCAAGTTCA
TGCCACCACCGACTTATACTGAGGTGGATCCCTGCATCCTCAACAACAAT
GTGCAGTGAGCAT
nt: 692
SEQ ID NO: 418
TGCAGAGGGGTCCATACGGCGTTGTTCTGGATTCCCGTCGTAACTTAAAG
GGAAACTTTCACAATGTCCGGAGCCCTTGATGTCCTGCAAATGAAGGAGG
AGGATGTCCTTAAGTTCCTTGCAGCAGGAACCCACTTAGGTGGCACCAAT
CTTGACTTCCAGATGGAACAGTACATCTATAAAAGGAAAAGTGATGGCAT
CTATATCATAAATCTCAAGAGGACCTGGGAGAAGCTTCTGCTGGCAGCTC
GTGCAATTGTTGCCATTGAAAACCCTGCTGATGTCAGTGTTATATCCTCC
AGGAATACTGGCCAGAGGGCTGTGCTGAAGTTTGCTGCTGCCACTGGAGC
CACTCCAATTGCTGGCCGCTTCACTCCTGGAACCTTCACTAACCAGATCC
AGGCAGCCTTCCGGGAGCCACGGCTTCTTGTGGTTACTGACCCCAGGGCT
GACCACCAGCCTCTCACGGAGGCATCTTATGTTAACCTACCTACCATTGC
GCTGTGTAACACAGATTCTCCTCTGCGCTATGTGGACATTGCCATCCCAT
GCAACAACAAGGGAGCTCACTCAGTGGGTTTAATGTGGTGGATGCTGGCT
CGGGAAGTTCTGCGCATGCGTGGCACCATTTCCCGTGAACACCCATGGGA
GGTCATGCCTGATCTGTACTTCTACAGAGATCCTGAAGAGAT
SEQ ID NO: 419
TTTTTTTTTTTTTCCTGCGGGAAAGCGCGCCATTGTGTTGGTACCCGGGA
AATTCGCGGCCGCGTCGACACAGGCCCCAGCATCAAGATCTGGGATTTAG
AGAGGAAAGATCATTGTAGATGAACTGAAGCAAGAAGTTATCAGTACCAG
CAGCAAGGCAGAACCACCCCAGTGCACCTCCCTGGCCTGGTCTGCTGATG
ACACAGGTTGGGCNGGNNCNCNGGGGNGGNNNNGNNNNGCNGNNGGNNCN
GNNNNCNNNNNGCNNNNGNNNNTNNNCNNNGNNCNNNNNNNNNNNNNNNN
NGNTCNNGNNGCNGGGGCCNGGNCGNCGCGGNCGCGNNTNNNNGGGTNCN
NNCNCNNNGGCGCGC
SEQ ID NO: 420
CAGACTCTGACCCAGCCTCAGTCCTAACTCCTGGGGCTGGGCTGAGGGGA
ACAAGCATTTGCTGAAACTTGAAAAAACAAAGCAAATCAAAAACAGGAAA
AAATTGTACCTGGTACTTTTTTTTAGAAAAAAAGATTAAAAAAGAAAGAA
TAAATTCTTGTTTGGAAACTTGAAAAAAAAAAAAAAAAAAAAAAAAAAAA
AAAAAAAAAAAAAAAAAAAAAAAAAAAAAATTTTAAACTCTNNNNNTNNC
NNCNANTAANNCANNTCNANNNNANNNAATTACTTNNANGTNNNTCACN
nt: 642
SEQ ID NO: 421
ACGAGAAGCCAGATACTAAAGAGAAGAANCCCGAAGCCAAGAAGGTTGAT
GCTGGTGGCAAGGTGAAAAAGGGTAACCTCAAAGCTAAAAAGCCCAAGAA
GGGGAAGCCCCATTGCAGCCGCAACCCTGTCCTTGTCAGAGGAATTGGCA
GGTATTCCCGATCTGCCATGTATTCCANAAAGGCCATGTACAAGAGGAAG
TACTCAGCCGCTAAATCCAAGGTTGAAAAGAAAAAGAAGGAGAAGGTTCT
CGCAACTGTTACAAAACCAGTTGGTGGTGACAAGAACGGCGGTACCCGGG
TGGTTAAACTTCGCAAAATGCCTAGATATTATCCTACTGAAGATGTGCCT
CGAAAGCTGTTGAGCCACGGCAAAAAACCCTTCAGTCAGCACGTGAGAAA
ACTGCGAGCCAGCATTACCCCCGGGACCATTCTGATCATCCTCACTGGAC
GCCACAGGGGCAAGAGGGTGGTTTTCCTGAAGCAGCTGGCTAGTGGCTTA
TTACTTGTGACTGGACCTCTGGTCCTCAATCGAGTTCCTCTACGAAGAAC
ACACCAGAAATTTGTCATTGCCACTTCAACCAAAATCGATATCAGCAATG
TAAAAATCCCAAAACATCTTACTGATGCTTACTTCAAAAAGA
SEQ ID NO: 422
CCCTATACCTTCTGCATAATGAATTANCTAGAAATAACTTTGCAAGGGAG
AGCCAAAGCTAAGACCCCCGAAACCAGACGAGCTACCTAAGAACAGCTAA
AAGAGCACACCCGTCTATGTAGCAAAATAGTGGGAAGATTTATAGGTAGA
GGCGACAAACCTACCGAGCCTGGTGATAGCTGGTTGTCCAAGATAGAATC
TTAGTTCAACTTTAAATTTGCCCACAGAACCCTCTAAATCCCCTTGTAAA
TTTAACTGTTAGTCCAAAGAGGAACAGCTCTTTGGACACTAGGAAAAAAC
CTTGTAGAGAGAGTAAAAAATTTAACACCCATAGTAGGCCTAAAAGCAGC
CACCAATTAAGAAAGCGTTCAAGCTCAACACCCACTACCTAAAAAATCCC
AAACATATAACTGAACTCCTCACACCCAATTGGACCAATCTATCACCCTA
TAGAAGAACTAATGTTAGTATAAGTAACATGAAAACATTCTCCTCCGCAT
AAG
nt: 620
SEQ ID NO: 423
CTCTCCTGTCAACAGCGGCCAGCCTCCCAACTACGAGAATGCTCAAGGAG
GAGCAGGAAGTGGCTATGCTGGGGGCGCCCCACAACCCTGCTCCCCCGAC
GTCCACCGTGATCCACATCCGCAGCGAGACCTCCGTGCCCGACCATGTCG
TCTGGTCCCTGTTCAACACCCTCTTCATGAACACCTGCTGCCTGGGCTTC
ATAGCATTCGCCTACTCCGTGAAGTCTAGGGACAGGAAGATGGTTGGCGA
CGTGACCGGGGCCCAGGCCTATGCCTCCACCGCCAAGTGCCTGAACATCT
GGGCCCTGATTTTGGGCATCTTCATGACCATTCTGCTCGTCATCATCCCA
GTGTTGGTCGTCCAGGCCCAGCGATAGATCAGGAGGCATCATTGAGGCCA
GGAGCTCTGCCCGTGACCTGTATCCCACGTACTCTATCTTCCATTCCTCG
CCCTGCCCCCAGAGGCCAGGAGCTCTGCCCTTGACCTGTATTCCACTTAC
TCCACCTTCCATTCCTCGCCCTGTCCCCACAGCCGAGTCCTGCATCAGCC
CTTTATCCTCACACGCTTTTCTACAATGGCATTCAATAAAGTGTATATGT
TTCTGGTGCTGCTGTGACTT
SEQ ID NO: 424
TTCGTAATTAGAATACTGTTTGGACTTGCTCAACAAGCACCTTATCTTAA
CAAAAAGTAACTTATAGAAAAGGGAGACATTCATTTAACTTCAAGCCCAT
ATTATTCTTAAAAGCTGACTCTTGAAATAGTATTTATTGAGTCATAGTGG
AGTCATGGGACTTTTTAAGGGCCGGAAGGGACTATTTAGATCATCCAGTC
CCACCCTGTCATTTTATGGAGGAGGAAACTGAGGCCTAGATAAGATAACC
AGTTAGTGGGTCCACTGACCTTTAGGACAGTAGTCTATCCGTAAGAGACA
ACATGGAGAAAGAAATACAACGTTTTTATAGTGAATTATCATCTTACAAA
GAATATTCTTCCCATATCGCACTTTTAAAAAGTGGGTACCTTAGTCAAAT
AGGAGAAAAAACCACTTGAGTAGTTTCATCCTCAGGTTTTAGGTGAGGAA
ACTGATACTCAGATTAAATAACTTTAAGCACACAGAGCCTGAATGATAGT
CTTATTTGAGCTCATCTGTGCTTTTAATGTGTACTACGTTAGGTGTTTTC
ACTTGCATTTCCTTTAGTCTTATTTGAGCTCATCTGTGCTTTTAATGTGT
ACTACGTTAGGTGTTTTCACTTGCATTTCCTTGTTTGACGTTGACAATAA
ATCGTGAAGCTGCCTTATCTAAGGAAGTCCTAAAGTAAATCATTGGAACA
CA
SEQ ID NO: 425
CCATTGTGTTGGNACCCGGGAATTCGCGGCCGCGTCGACGGAGTTTTACC
TTATTACACTTTAATCTCTGGATTTACCCCATCTCATTTCTCTTTTAGGA
AAACTGTTTGTATGTGGTGGCTTTGATGGTTCTCATGCCATCAGTTGTGT
GGAAATGTATGATCCAACTAGAAATGAATGGAAGATGATGGGAAATATGA
CTTCACCAAGGAGCAATGCTGGGATTGCAACTGTAGGGAACACCATTTAT
GCAGTGGGAGGATTCGATGGCAATGAATTTCTGAATACGGTGGAAGTCTA
TAACCTTGAGTCAAATGAATGGAGCCCCTATACAAAGATTTTCCAGTTTT
AACAAATTTAAGACCCTCTCAAACTAACAGGCTTAGTGATGTAATTATGG
TTAGCAGAGGTACACTTGTGAATAAAGAGGGTGGGTGGGTATAGATGTTG
CTAACAGCAACACAAAGCTTTTGCATATTGCATACTATTAAACATGCTGT
ACATACTTTTTGGGTTTATTTGGAAAGGAATGCAAAGATGAAGGTCTGTT
TTGTGTACTTTTAAGACTTTGGTTATTTTACTTTTTGGAAAAGAATAAAC
CAAGAATTGATTGGGCACATCATTTCAAGAAG
nt: 374
SEQ ID NO: 426
AGAGCAGCAGCCATGGCCCTACGCTACCCTATGGCCGTGGGCCTCAACAA
GGGCCACAAAGTGACCAAGAACGTGAGCAAGCCCAGGCACAGCCGACGCC
GCGGGCGTCTGACCAAACACACCAAGTTCGTGCGGGACATGATTCGGGAG
GTGTGTGGCTTTGCCCCGTACGAGCGGCGCGCCATGGAGTTACTGAAGGT
CTCCAAGGACAAACGGGCCCTCAAATTTATCAAGAAAAGGGTGGGGACGC
ACATCCGCGCCAAGAGGAAGCGGGAGGAGCTGAGCAACGTACTGGCCGCC
ATGAGGAAAGCTGCTGCCAAGAAAGACTGAGCCCCTCCCCTGCCCTCTCC
CTGAAATAAAGAACAGCTTGACAG
nt: 567
SEQ ID NO: 427
GAATTATTGACTTTGAATTGCATTTCAGTACCATGAAGTCAAAGTCAGTG
GTGTATTTGCTCATTTGTTCATTCTTTCTTTTCCACCAACATTACTGCCT
GCAGAGCCAGAGGTGAGTGCAGAAATCCTGTCAATTCGTCACTTGTGGAC
AACCTGCAGCTTGCCACAGCCTACAGTTCCACCACTGTGACCTCTGAAAA
CCTCCTGAACAAAAGGAAGGAGACTTGGAAATCCTGAATGGGCTTGGAGA
CATTAAGGGAGAACTGCCTCCCTGGACCAAGGCAGAATTCAATAGAACCA
GCAAGAAATTTTCCTATGAATGGGAAAGCAGGTGGCAGGGGGCAGGGGTG
GAAAAGCTTTGTACAGGAATTGTGGAAAAGCTTTTGCATTATCTCTAGTC
TGAAAGTCACATTTCTCAGTTCCTTTCCACTCTCTTCTGTCAACTTGCTG
TGAGTAAATGACATCTGTCACCTGTGACACGGGCCAGGGACTATCACCAT
ATGGCCCCCACACATTATCTAGTACCAGCCTGCCTGGGCCATGCCTTTTC
CAGTCACTGTACCAGCC
nt: 620
SEQ ID NO: 428
CTCTCCTGTCAACAGCGGCCAGCCTCCCAACTACGAGAATGCTCAAGGAG
GAGCAGGAAGTGGCTATGCTGGGGGCGCCCCACAACCCTGCTCCCCCGAC
GTCCACCGTGATCCACATCCGCAGCGAGACCTCCGTGCCCGACCATGTCG
TCTGGTCCCTGTTCAACACCCTCTTCATGAACACCTGCTGCCTGGGCTTC
ATAGCATTCGCCTACTCCGTGAAGTCTAGGGACAGGAAGATGGTTGGCGA
CGTGACCGGGGCCCAGGCCTATGCCTCCACCGCCAAGTGCCTGAACATCT
GGGCCCTGATTTTGGGCATCTTCATGACCATTCTGCTCGTCATCATCCCA
GTGTTGGTCGTCCAGGCCCAGCGATAGATCAGGAGGCATCATTGAGGCCA
GGAGCTCTGCCCGTGACCTGTATCCCACGTACTCTATCTTCCATTCCTCG
CCCTGCCCCCAGAGGCCAGGAGCTCTGCCCTTGACCTGTATTCCACTTAC
TCCACCTTCCATTCCTCGCCCTGTCCCCACAGCCGAGTCCTGCATCAGCC
CTTTATCCTCACACGCTTTTCTACAATGGCATTCAATAAAGTGTATATGT
TTCTGGTGCTGCTGTGACTT
SEQ ID NO: 429
CACAAGATAGAATGGTAAAAAAAAAAAAAAAAAAAAAAAAAAAAAAAAAA
TTTTAAGTGACAGTGCCATAGTTTGGACAGTACCTTTCAATGATTAATTT
TAATAGCCTGTGAGTCCAAGTAAATGATCACTTTATTTGCTAGGGAGGGA
AGTCCTAGGGTGGTTTCAGTTTCTCCCAGACATACCTAAATTTTTACATC
AATCCTTTTAAAGAAAATCTGTATTTCAAAGAATCTTTCTCTGCAGTAAA
TCTCGCAGGGGAATTTGCACTATTACACTTGAAAGTTGTTATTGTTAACC
TTTTCGGCAGCTTTTAATAGGAAAGTTAAACGTTTTAAACATGGTAGTAC
TGGAAATTTTACAAGACTTTTACCTAGCACTTAAATATGTATAAATGTAC
ATAAAGACAAACTAGTAAGCATGACCTGGGGAAATGGTCAGACCTTGTAT
TGTGTTTTTGGCCTTGAAAGTAGCAAGTGACCAGAATCTGCCATGGCAAC
AGGCTTTAAAAAAGACCCTTAAAAAGACACTGTCTCAACTGTGGTGTTAG
CACCAGCCAGCTCTCTGTACATTTGCTAGCTTGTAGTTTTCTAAGACTGA
GTAAACTTCTTATTTTTAGAAAGTGGAGGTCTGGTTTGTAACTTTCCTTG
TACTTAATTGGGTAAAAGT
nt: 484
SEQ ID NO: 430
CAACCTTAGCCAAACCATTTACCCAAATAAAGTATAGGCGATAGAAATTG
AAACCTGGCGCAATAGATATAGTACCGCAAGGGAAAGATGAAAAATTATA
ACCAAGCATAATATAGCAAGGACTAACCCCTATACCTTCTGCATAATGAA
TTAACTAGAAATAACTTTGCAAGGAGAGCCAAAGCTAAGACCCCCGAAAC
CAGACGAGCTACCTAAGAACAGCTAAAAGAGCACACCCGTCTATGTAGCA
AAATAGTGGGAAGATTTATAGGTAGAGGCGACAAACCTACCGAGCCTGGT
GATAGCTGGTTGTCCAAGATAGAATCTTAGTTCAACTTTAAATTTGCCCA
CAGAACCCTCTAAATCCCCTTGTAAATTTAACTGTTAGTCCAAAGAGGAA
CAGCTCTTTGGACACTAGGAAAAAACCTTGTAGAGAGAGTAAAAAATTTA
ACACCCATAGTAGGCCTAAAAGCAGCCACCAATT
SEQ ID NO: 431
GACAGGCGGGGGCCCAGCGGCCGGGTGAAGGCCGGGTGGCTCTGTGAATC
AAAGGAGAGTCCCAGAAAACCTGTGACTGTTGAAGAAAATTCATCTGTGA
ATTTTTATATTCAAGGAGTCAGTATTTATATTCATCTTTTAAACTGGGAA
GATTTATATTTTACTTTAAAACTTCTTGATAATAATTTACAATGAATGGA
CACAGTGATGAAGAAAGTGTTAGAAACAGTAGTGGAGAATCAAGGTAAGT
AAGCACTTTGTTATCAATTGTTTACTATGAAGAGAGTTGAAAACTTGACT
TTTTTCTTTATTGTTATTGTTGTTATTTAGTTTTCCTCATAGGTAGCAGA
GTTTTCAGGTTTTCCTCTTAGCTATCCAAATACTAAAAAAATTCTGATAT
ACGAACCTTTTTTCATAATACAGGTTTTAATTATATTTTTCATTCAGATA
CACAGTAGATCTTAAATATAGAAAGTTTTTGTTTACTTAAATCTATTTGG
AAGTTTATATTTGAGCTAATAATTAAGCTGGAGCATGTATAATAGATTTA
AATTGTTTTGACTGTTAGTGAAATTT
SEQ ID NO: 432
CTCACTTGGTGGGTGAGCCTCCAATGACTACACCCAAGGAGGATTTAACA
CAGGGATTTTATGACTTGCAACAAGTCAGGAGGACATGGGGTTGGGGTAG
TTCAGCAGTGCCTGTCTGAACAAAGGTGAAAATTGGGCTTTTATTGGGCT
GATCAAGGGGGAGTAAAGGCAGCCAGGAGCAGTCGCCTGTCATGCTTCTA
CCTATATTGCATGTATAGAAAAGGGAAAATAAACTCCTTCCTGGGCAGGG
TTTTAGTATGCTAAGGAGGGGAGTTATTCAACTTCAATCCAACTCAAGCA
TCAGCATTGCTGCGTCCATCCCAGTTTTGTTTTGCTGGGGCTGAACTTCT
TCCTATAACTTTTTGAAACAACAAGAACTCAAGGTGTGACAGTTACAAGT
GGGCCCTTTTTCACAGTGTGTACCTAAACACGTGAGGACCCTGGATTACA
GAATGACAGACTCGAAGTGACTCAAGTTCCGGTTGTTCATCTTTAGATGG
TAAAGATGGCTGTACGTACTATCCTTGCTTATTTCCAATCTATTGTTTAA
ACTCTTGTATATGTAATACCGCAGAGGCTAGAGATACAACCTTTGACCAA
ATGAGTGAATTCAAGTAATCCATTACTAATGTGATCTGGAAACAAACATG
GTGTTGAATGTGCATATGT
nt: 559
SEQ ID NO: 433
CTTGGCAGCTCCGTTATGTGCCCAGCTCTTTGCAAGGGCATACTGGGAAA
TGAGTGGAGATAAAGGACCCAATCATAAGCATTTTACAGTATGGATACCC
CATTTTAAAAAGGTAAACTGAGGCACAATGCAATTTTTTTTTTTTTTTAA
GGAGTTTATTTGAGCAAACAGTGATTCATGAATCAGGCAGCACCAAACCA
GAAGGAGGCTTTGCTGAANAAGGATGAGGGACAAGCATTTATAAAGTGAA
TGTAGATGTAATACAAAGAAAATATTTGAACCGGGTGCGGTGGCTTACAC
TTGTAATCCCAACACTTTGGGAGGCCAAGGCGGGCAGATCACAAGATCAA
GAGATCGAGACCATCCTGGTCAACATGGTGAAACCCCATCTNTACTAAAA
AATACAAAAATTANCTGGGCGTGGTGGTGCGTGCCTGTAGTCCCAGCTAC
TTGGGCGGCTGAGGCAGGANAATTGCTTGAACCCGGGAGGTGGAGGTTGC
AGTAAGCCGAGATTGCACCATTGCACTACTCCAGCCTGGTGACAGAGAGA
GACTCCATC
SEQ ID NO: 434
GANNNGTGCGATANNATGNNTGTCTTTTTTTTAAAGTNTTTCNNATNGNA
GNGAANCCCCCNNANNTNNCATAANGAGAGATNACTACNGTACANATAGN
GNCANACNGATAGTAGTANCAANATTGTNTTAGCTANATNANTCAATAGA
TATCNAGATANAANAANANCNNGGATATACAGCGATGTNTNANNGGNNNN
NNNANGGAACGAACATCNACNTTAANNATAAGCTNGNGGAGAGAGACANG
TANGTTATANANNAGAATNGNAGTAGGNGTGATCATAATAGNNNNNANNT
ANTATATANGATNTTANTGNNCTNTNNTNNGTTTATCNNNAATNTCTATN
CTNGAGAGNAGCNNNATNNNNAGGCGANGANATTGGGNNNTNCTCNTNAT
AGANANCTGGTGTCNNANAANTACNTCATCTATTNANCTCTCACNANATG
GNANNATANAGNAGNGNNNTNNANAGGANTANGCATAGNGNNTNNCTNAA
ACAAAANNNATAAGANNTCTCGNNAANANGGGCCTNTNNTNTAGCGAGGN
NTTANTTTNTATANTTNTTCNCTCTTNNAATANNTANGATANATGANCTN
GNNGTGATANATANNNNNTACNGTNAANNTNTANTCNTATAATAGATANA
AATATAGGATNTTNCTCTGGCNGGTNGAANANTTNNTNCNNTTTNAATAA
TGNTGTTAGNGACNGNGNTNTNANANNNNNTTAGAAAGGTACTCTATATA
CTNNTATGNTNCGGCNNATAATANAACAGATGTTTGTATNAATATNAAAN
AAGGTCNNTTTCGNCAAGAGAANNNTGNCTGGTNATAGAATTAGCATAAN
TTANNTANTATGATNNANTNNTNCTACNANTNTTAGCNNTTNGCAGNAGT
CATTNNGNATNTATNNNGNNTANTAGTNANTTGGGNCTNNTNCAGANTAT
ATTNTGNGAANATGAANNTACGNANTCCTNNGNANTATNATNNTGANTAN
GANAANCNANANNTNTTNTANNANTGNCTATANATTGCCNNGATANATTN
TNNNAATGAANCGATAGCCCGCNCTAAGGANNTNNGTNANNTAAANNTCT
CAGATAANNTACNTNTTNNTTATTAANCNANNATCACANTATANCNGNGA
CANNNGCGANANTATATGTATGNNANTATNACNGNTCCNNNCCGNGAANN
TANTCNTANNAGGCATTCNGNNGAGCTNTTCTNCTAGACNATTTNNANTG
AAANNATGCNGNNAAAAACGACNNNCTTNAANTTNTGTCTACANTCCGCN
NTNTTTNTACAGATNGCAGNTAAGNNNANTNANNGCTCTCANCTNGCTNN
NACT
nt: 741
SEQ ID NO: 435
AAGCAGAANTNTCTCTAAAAACATTATCTCCTTAAAATCTTGAGGTGCAT
ATNAGAGCCACAGGCAATCTCTGACATATAAAATTGCAGTACAGGCCTTT
CAAATTTGGCATTTCACTGGTACAATACAACAACCAAGATATATAATAAC
TGTACAGTGCCTAGACATTCCAGTAAGAACCATTATTTTCTTTAATGTAG
AATGATTAATACATATTCTACAAGGGGCAGTAAGGTTAGTAATTCTATAG
GGTATGTCCCGACATAATTTTCAAATTGTACAATAACACAAACAACTTTG
TTAAGGCCATGTTTTATTTGCTGATTAATGGACAAAAGGCAATGTAATTT
ATTTTCAAGTATTTTCTTGAAAGTCTGTGCTCATAAAAATCATGAAAAGT
TGGAAAGACTGTTAAATCACTGAAACTTCAAATATATCTTACACAATCTT
GTTTGTACAAAAATACAAGTTAAATATAAACATAAAGCAATCATGGTAAT
TTTATGCAAATCTGTTTTATGTGATCATCAGTTATATATAAAAGTTTCTC
AGTTCTGTTATTTGTGAAAAGATCAATACCAGATTGAATGACTACCTATT
GGCAAAGGGCCCTAAAAAGCTTACTTTAGCACTCATCTTTTACATGGTTA
AATGCATTTCCTAATTTGAGATCACCTAAACACTGGAAAAGAAAAAAAAT
GAAAGGGCAGTATGTCCATAAACCAACAAATAATTTGGCTG
nt: 485
SEQ ID NO: 436
CGAAATTTCCTTGTGACACAGAGGAAGGGCAAAGGTCTGAGCCCAGAGTT
GACGGAGGGAGTATTTCAGGGTTCACTTCAGGGGCTCCCAAAGCGACAAG
ATCGTTAGGGAGAGAGGCCCAGGGTGGGGACTGGGAATTTAAGGAGAGCT
GGGAACGGATCCCTTAGGTTCAGGAAGCTTCTGTGCAAGCTGCGAGGATG
GCTTGGGCCGAAGGGTTGCTCTGCCCGCCGCGCTAGCTGTGAGCTGAGCA
AAGCCCTGGGCTCACAGCACCCCAAAAGCCTGTGGCTTCAGTCCTGCGTC
TGCACCACACAATCAAAAGGATCGTTTTGTTTTGTTTTTAAAGAAAGGTG
AGATTGGCTTGGTTCTTCATGAGCACATTTGATATAGCTCTTTTTCTGTT
TTTCCTTGCTCATTTCGTTTTGGGGAAGAAATCTGTACTGTATTGGGATT
GTAAAGAACATCTCTGCACTCAGACAGTTTACAGA
SEQ ID NO: 437
GGTTTTTATACTTGCCATGAAACTGTTCTTTGGGATATTATTTTGTTCAG
GTTCCCCACTTGGACAGCAGAGGGGGTGACTCTGCCCATCCCTGCCACTG
GTAGCCAGGCGGGCAATGTCTGCTAGCAGTCTGCTTCTGTCTGAACTCAG
CCAGCAGAGGCAAACTCCCGGTTCCCCGAGAAACACTCTGAAGGCAGGGT
GGGTGACTCCACCCACCACCGCCTCTCCTAGCCATGCAGGCCATGTCTGC
TAGAGCTTCCAGCGCAGTGGTCCTAATTCTGTCTGAATCCGGCTGAGGGG
TGCAGCCTCCTGTTACTGCCCAGGGAAACACCCAGATGGCAGGGTGGGTG
ACTCCAACCACCTCTGCCTGTGGTAGCCAGATGGGCCACACCTGCTAGAG
CTTCCAGCCCAGCAGTCCCGCTACTCTGTGGGTGGGTGCCATCCCCTGTT
CCTCTGGGAAGCACCCAGACAGCTGATTACGTGACCCCACCCACTTCTGC
AGATCCTAGCTGAGCAGGACTTGCTGGTTTGGACAATGCCCAAGCAGGGA
AGAGCCCTCATTCTCTTATCACTGACAGAGGTGAGATGTCCGANTTTGTA
NGCTGGTGGAGGAGTGAGGTGGAGGAGGTATGCCTCT
SEQ ID NO: 438
GTTATTCAGGTATCCATCAAAATTTTATAAGAGGGCCGGAAACATCGGCT
CACACCTGTAATCCCAGCACTTTGGGAGGCTGAGGCAGGTGGTTCACTTG
AGGTCAGGAGTTCGAGACCAGCCTGGCCAACATGGCAAAACCCCGTCACT
ATTAAAAATACAAAACATTAGCTGGGTGTAGTGGCAGGTGCCTGTAATCC
CAGCTATTCGGGAGGCCTAGGAAGGAAAATGGCTTGAACCTGGGGGTGGA
GGTTGGAGTGAGGCAAGATCACACCACTGCACTCCAGCCTGGGCGACAGA
GCGAGACTCCATCTCAAAAGAAGAAAAAAAAAACAACAAAAAAACCTTTA
TCAGATTATCAGAGGTTATCACTACAGAGGGAGGTAAAATTGGAGGGAAA
AGGGTACAAATTTATTTCAC
nt: 741
SEQ ID NO: 439
AAGCAGAANTNTCTCTAAAAACATTATCTCCTTAAAATCTTGAGGTGCAT
ATNAGAGCCACAGGCAATCTCTGACATATAAAATTGCAGTACAGGCCTTT
CAAATTTGGCATTTCACTGGTACAATACAACAACCAAGATATATAATAAC
TGTACAGTGCCTAGACATTCCAGTAAGAACCATTATTTTCTTTAATGTAG
AATGATTAATACATATTCTACAAGGGGCAGTAAGGTTAGTAATTCTATAG
GGTATGTCCCGACATAATTTTCAAATTGTACAATAACACAAACAACTTTG
TTAAGGCCATGTTTTATTTGCTGATTAATGGACAAAAGGCAATGTAATTT
ATTTTCAAGTATTTTCTTGAAAGTCTGTGCTCATAAAAATCATGAAAAGT
TGGAAAGACTGTTAAATCACTGAAACTTCAAATATATCTTACACAATCTT
GTTTGTACAAAAATACAAGTTAAATATAAACATAAAGCAATCATGGTAAT
TTTATGCAAATCTGTTTTATGTGATCATCAGTTATATATAAAAGTTTCTC
AGTTCTGTTATTTGTGAAAAGATCAATACCAGATTGAATGACTACCTATT
GGCAAAGGGCCCTAAAAAGCTTACTTTAGCACTCATCTTTTACATGGTTA
AATGCATTTCCTAATTTGAGATCACCTAAACACTGGAAAAGAAAAAAAAT
GAAAGGGCAGTATGTCCATAAACCAACAAATAATTTGGCTG
nt: 203
SEQ ID NO: 440
TTGAGGAAGGGTCTACTGTCTTTTTAAATGGCACAATTTTAAGAGGTTTG
AGAGGTACAGTCCCTTAACCTGCCACGGGAGAGGGGCCCCCAAACTTTCT
TCCCCCCACACTTCTGGTTTTCTGTGTGGAGGGGGAGCAGGGATATCTAA
GCTGTGGTGTGAAAGGGTAGGAGAGATGCTGGAGGTGGGGGTGCTGTGTT
CTA
SEQ ID NO: 441
TTTCCTCGGGAAGCGCGCCATTGTGTTGGTACCCGGGAATTCGCGGCCGC
GTCGACATTTTTTTTTTTTTTTTTTTTTAGAATGATTAACAATTTATTGA
GTTTTATTTATCTACAAAAATATAGCAATACAGNGAACTTCACCAAATCC
TAAATATTCAGTACCTGAACTGGCTACAACACCGNGTGCACACCCAGTTC
CTGCAGAATCTCTTGCAGATATGGGAGAGTCAGCCAGTGAAAAGATCCAT
TTCTTGGGAATCCTTGTCAACAAGACCAGTTCAGAAATCCAGGATATATA
GAAGCCTACTGTAATTTAAAAACAGTAACAAAAACCCCAACAAAACCCAA
ATCAACAAAGACCAAGATAAAGGNGTGATAAACATTAATTGTAATGGTTT
TCCTTTACATGCAATACATGCATTTTAAAATCACTAAGAAACACGAAATT
TTGTAGAGCAAAGTTTGNGTTTCACGTAAGTGCAAATGAATATATATTTT
ATTTTTTATACTATTAAATTATATATATTTTTTCCATACAAAAGCACACA
GTGTTAATCTATAAAATGACATCCAAGTGGATGATGATTGTTTTTGCATG
TCCCCCTGCTTAGATTTTTTTAAAATATATAGTCAAAAATTAACATCCTT
CTTTAAAAATACAGAAGGGAAAAANGGGCAAAAAAAAAAATCTAGACTCG
AGCAAGCTTATGCATGCATGCGGCCGCAATTCGANCTCGGNCGACTTGGC
CAATTCGCCCTATAGNGAGTCGNATTACAATTCACTGGGCCGNCGNTTTA
CAACGTCGNGACTGGGAAAACCCTGGCGTTACCCNNCTNATCGNCTTGNA
ACAATNCCCNTTTNGCCAGNGGGG
SEQ ID NO: 442
TCACTTCGTATNGAANCTGTTTGGACTTGCTCAACAAGACCTTATCTTAA
CAAAAAGTAACTTATAGAAAAGGGAGACATTCATTTAACTTCAAGCCCAT
ATTATTCTTAAAAGCTGACTCTTGAAATAGTATTTATTGAGTCATAGTGG
AGTCATGGGACTTTTTAAGGGCCGGAAGGGACTATTTAGATCATCCAGTC
CCACCCTGTCATTTTATGGAGGAGGAAACTGAGGCCTAGATAAGATAACC
AGTTAGTGGGTCCACTGACCTTTAGGACAGTAGTCTATCCGTAAGAGACA
ACATGGAGAAAGAAATACAACGTTTTTATAGTGAATTATCATCTTACAAA
GAATATTCTTCCCATATCGCACTTTTAAAAAGTGGGTACCTTAGTCAAAT
AGGAGAAAAAACCACTTGAGTAGTTTCATCCTCAGGTTTTAGGTGAGGAA
ACTGATACTCAGATTAAATAACTTTAAGCACACAGAGCCTGAATGATAGT
CTTATTTGAGCTCATCTGTGCTTTTAATGTGTACTACGTTAGGTGTTTTC
ACTTGCATTTCCTTTAGTCTTATTTGAGCTCATCTGTGCTTTTAATGTGT
ACTACGTTAGGTGTTTTCACTTGCATTTCCTTGTTTGACGTTGACAATAA
ATCGTGAAGCTGCCTTATCTAAGNAGTCCTAAAGTAAATCATTGGAACAC
ATGTANCCAGTTTGTTGTTTTTATTTGCCAGGTNTCAAATATAACTGAAA
ACCCATGCTAACTGACTNATTTTAAAAGNTGTNTGGGGCATGAAANGATT
GCTCTGCCTGGGCGGGNGGTTNANCCTGNGTCCCCCNTTTNGGAGNCCAC
CCANGANGCGATATTTNAGGGNNGATTCNAAACCCCTGGCACGNGNNAAC
CCCNTTTTTAAANANAAAANANCGGNNG
SEQ ID NO: 443
TTGTGTTGGTACCCGGGAATTCGCGGCCGCGTCGACGGAGTTTTACCTTA
TTACACTTTAATCTCTGGATTTACCCCATCTCATTTCTCTTTTAGGAAAA
CTGTTTGTATGTGGTGGCTTTGATGGTTCTCATGCCATCAGTTGTGTGGA
AATGTATGATCCAACTAGAAATGAATGGAAGATGATGGGAAATATGACTT
CACCAAGGAGCAATGCTGGGATTGCAACTGTAGGGAACACCATTTATGCA
GTGGGAGGATTCGATGGCAATGAATTTCTGAATACGGTGGAAGTCTATAA
CCTTGAGTCAAATGAATGGAGCCCCTATACAAAGATTTTCCAGTTTTAAC
AAATTTAAGACCCTCTCAAACTAACAGGCTTAGTGATGTAATTATGGTTA
GCAGAGGTACACTTGTGAATAAAGAGGGTGGGTGGGTATAGATGTTGCTA
ACAGCAACACAAAGCTTTTGCATATTGCATACTATTAAACATGCTGTACA
TACTTTTTGGGTTTATTTGGAAAGGAATGCAAAGATGAAGGTCTGTTTTG
TGTACTTTTAAGACTTTGGTTATTTTACTTTTTGGAAAAGAATAAACCAA
GAATTGATTGGGCACATCATTTCAAGAAGTCCCCTCTCCTCCACATTTGT
TTTGCCAATTTGCACATTAAATGACTCTTCCCTCAAATGTGTACTATGGG
GTAAAAGGGGTAGGGNTTAAANATGTAAACAGTTGGGTTTTTTAAGGGNC
CTTTTTCATAACTGGAACACTCTNTACAAGGNTNCTTNTTAAATAAATAA
CTTGACTTTTTTGTTTTNTAAANGNANCTTCNTGCTTCCATAAAAAAAAA
AATTTAANTNGNCANCTNTGCTGCTGCGNCCANTTNGCTNGNCCNTGGCA
TTCCCTAGGGANGNTNAATANTGGCNNNTTAACNNGGCNGNAACNNNNNC
CANT
SEQ ID NO: 444
GGGCGATGCATGCTTTATTAAGGCTCTTGTTTCACCTGGCAGTGTACTGT
ATCAACGTATAATACAGAAAAAAAATCTCTTTAAGGTCCTCCTTCACAAA
GACATAGAGTGAAACTCCCTTTACATGTCAGTATTTGTTCAACACTTTAG
GCAACTTGACTGTCAGTGTTAAAATGGAAAACAGGAAAATGGAAAAATCT
GACCAATTCTGCCACCTTGAGACTTTCATATAGACCTTGCACAACAATTG
TATAGATCACACACCGGCTGTATTTAATATGTAACATTTTCACACATATT
AAAGATACAGAAGTATTAAAAAACCCCCAATGTTAATGTATTTGCTTAAA
AGGCACAAGTTTCACATATCTGTCTAGCTATCTGTTGGTAATACAGAAAG
TATACTACTTTTTTAAAAAAGTGGGCAGAATTCTTGTGTATGTATATTTG
TGTGTACAGTATGTGTATGTGTGTATATATATATATTATATATATAGATA
ATATATAAATATTTTTTTTAAGGAGAAACTAGAATGTTTAGCTAGAAAAT
TCCACAGCCTGTGAAGAAATATTTCAAAATGGCCATAAAGGAGGTAAAAA
TGAAAACCATAACCTAACTTTTATAGAGGCTTTATCTTTAATTTAACGAT
GTGCGGAGGACTTTCTTGCTTGAATCTGTTCCGGGCTGTCTGCTCTGTCC
ATCAAATGGGCAGGTCTGGGAATGAGGCACCTTCGGCCGTTCAGAAGTGG
CCTGAACAGAATGCTGGAACCCAGGCTGGACTCGGAC
SEQ ID NO: 445
CAAACCTGCATGTTCTGCACATGTATCCAGGAACTTAAAAAAAAAAAAAG
ATAGTTTGTGTGTCTTAATTGAATAATAGTAGATTTATAGATTAAAGATC
TATGGGTTTTTAATATGGATTAGAAATCTGTGGGTTTTTGATATGGATTA
GAAATCTGTGGGTTTTTAATATGGATTGGAAATCTGTGGGTTTTTAATAT
GGATTAAAAAACATCTGTGGGTTTTTAATATGGATTAAACATCTGTGGGT
TTTTAATATGGATTAAACATCTGGGTTTTTAATATGGATTAAACATCTGT
GGGTTTTTAATATGGGTTAAAAATCAAAAGAAAATGAACTATTTGCTCCA
GTGCAGGAAAATACAGGCAATACTGGATACAATTAGATGGTCAGGAGCGA
TAACCCGGTTGCCATTGTTTGAAGAAGAGAATAAGGTGCTAGCATTCCTA
TCCGTAGATAATTTGACAGCTAGGAAATAGGGGGAGTCTTCTATGTAGTT
AGTGAAGGCTAAATGAACTATTATATGCAGTTATCGTAGAAGAGTACTCA
AAAAAATCTGTAAAAAATAAAGAAAGGCCGGGCGCGGTGGCTCACGCCTG
TAATCCCAGCACTTTGGGAGGCCGAGGCGGGTGGATCATGAGGTCAGGAG
ATCGAGACCATCCTGGCTACCANGGTGAAACCCCCGTCT
SEQ ID NO: 446
CAAGACTCCATCTCAAAAAAAAAAAAAAATCTACAGTGCTGAGTATATAA
AATTATTAACACATTTCACAACAATATGTGTTTGTGGAGTTAAATATTTT
TTGTCTTTAAAACAGGTAATTTTAGTGCATACTTAATTTGATGATTAAAT
ATGGTAGAATTAAGCATTTTAAATGTTAATGTTTGTTACATTGTTCAAGA
AATAAGTAGAAATATATTCCTTTGTTTTTTATTTAAATTTTTGTTCCTCT
GTAAACTAAAAGAACACGAAGTAATTGGTCACAATTACTGGTGTTTAACT
GCCAAATATGGGTAAATAAGGGAAAATTTTGTTTAATATTTAGTCCTTCT
GAGATGGCTTGAATATTTGAATTTTGTTGTACGTCTATACTGGGTAGTCA
CAAGTCTTATAAACACTTTAGAGGAAAGATGGATTTCAGTCTGTATTTTT
AAACATCATTTATTTTAAATCTGGTGCTGAAAAATAAGAAAAAAATTAAA
CTGCATTCTGCTGTTCTTCTTTAGAAGCATTCCTGCGTAAATACTGCTGT
AATACTGTCATGCAAAGTGTATCCTTTCTTGTCGTATCCTTTTTGGGGCA
GTGTTTTTTTGTTTTTTTCCTAGAAATGTTTGTCCTTCCCCCACCTGTTG
ATCCAGGTTAAGGAATACTTTTTTACACTTTATTCAAA
SEQ ID NO: 447
CTTTTCCTCCCGCTGTCCCCCACGGAGGGGACTGCTCTCCCCCGCTGCAT
CCTTTCTGTGAGGTACCTTACCCACCTCAGCACCTGAGAGGGTGAAATAG
AATTCTAACCTCGACATTCGGGAAGTGTTTTTGAGAAGTCTCGGTCGGTA
AGGGAAGTCTTCCAAGTCCGTGCAGCACTAACGTATTGGCACCTGCCTCC
TCTTCGGCCACCCCCCAGATGAGGCAGCTGTGACTGTGTCAAGGGAAGCC
ACGACTCTGACCATAGTCTTCTCTCAGCTTCCACTGCCGTCTCCACAGGA
AACCCAGAAGTTCTGTGAACAAGTCCATGCTGCCATCAAGGCATTTATTG
CAGTGTACTATTTGCTTCCAAAGGATCAGGCCCTGAGAACAATGACCTTA
TTTCCTACAACAGTGTCTGGGTTGCGTGCCAGCAGATGCCTCAGATACCA
AGAGATAACAAAGCTGCAGCTCTTTTGATGCTGACCAAGAATGTGGATTT
TGTGAAGGATGCACATGAAGAAATGGAGCAGGCTGTGGAAGAATGTGACC
CTTACTCTGGCCTCTTGAATGATACTGAGGAGAACAACTCTGACAACCAC
AATCATGAGGATGATGTGTTGGGGTTTCCCAGCAATCAGGACTTGTATTG
GTCAGAGGACGATCAAGAGCTCATAATCCCATGCCTTGCGCTGGTGAGAG
CATCCAAAGCCTGCCTGAAGAAAA
SEQ ID NO: 448
CAAGAACTCTGGGACATTTGCAAAGGGTATGGCATATGTGTAATGGGAAT
ACCAGAGGAGAGGAAAGACAGGAAGTCAAAAAAAGAATTTTTCCAAATTA
ATGATAGGTTCCAAACCACAGATGCAGGAAGCTTAAACACCAACAGGATA
AATAAAACAAAATCTACGCTTAAGCATATCATACTTAACCTGCAGAAAAT
TACAGACAAAGAAAAAACACCAGAGGGGAAGCTGGCAGAAACATACCACC
TATAGCGGAAGAAGAATAAGAATTACATCAGACTTCCCTTCAGAAATCTT
GCAAACAAAAAGATGTAGCACAATATTTAAAGTATTAAAGGAGGCCGGGC
CCGGTGGCTCGGGCCTGTAATCCTAACACTTTGGGAGGCTGAGGCAGGAG
GACCATGAGGTCAGGAGATCGAGACCATCCTGGTGATGGTGATACCCCAT
CTCTACTAAAAATACAAAAAATTAACCGGGCATGGTGACACGCACCTGTA
ATCCCAGCTACTTGGGAGGCTGAAGCAGGAGAATCGTTTGAGCCCAGGAG
GTGGAGGTTGCAGTGAGCCGAGATCACATCACTGCACGCCTGGGCAACAG
AGCGAGACTCCATCTCAAAAAA
SEQ ID NO: 449
CGACCCGTTTTAGTCAGGATGGTCTCGATCTCCTGACCTCGTGATCCGCC
TGCCTCGGCCTCCCAAAGTGCTGGGATTACAGGCGTGAGCCACCGCGCCC
GGCGTAAATCAGGTTTTTTAAATGTTTGCCAAACCTTATCACTGACTTTT
ATAACAAAATTATTTACTATAATCATTAGGGAATATTTAAGTTCTGCTAA
TACTTAAAATTGCAGAGTGCTAAAACCAGCAGTGAGTTTAGAATCAAGCT
AAGCTTTATTGTTGCTACTATTTGAGGCATATTAGTTGACTGGTGTTCAT
ATGCAAGGCAGTCTACTGGGTGCAACAAGGGTTAGAAGGATATTTTTAAA
AAACTGACCCTATTCTCAGGATGAAAATAATACACTAGTAATAGTCTGCT
CTGTTGGTTAACTCCTCGTAAGGAGGTACAATTAAAATGCTGTAGTGTTG
CAAGGGAAGGAGAGGAAGAATCATATTCCTTCACTAGCAGGATCAAGAAA
GCTTTTATAGAAATATACAAAATCTTCACTTCTTGAAGGATTGGTAAAAT
TTAATAGCCAACATTGGGCACTTATTCATTCTCTGAGTAAATATTTATTG
CATGCTTATCTTGTATCAAGCATTGTGATGAAAGCACAAGAATGAAAGAG
GAGGGAGAATGTTTAGAGAATAAGGGCTGAAACACAGATTTTGTAGGGAG
CGTAGGGGAGACTGANAAGACAGGTTCAGGTTAGTAAGGGCGCTCATATT
TTGACCCTGAATGTTAACTATGTGCACATCATGCTAGCTATTCTAAATCA
GGCATTTTCAAATGGAAGCAGGCACTGACATTTT
SEQ ID NO: 450
CGTGAAGGGTCTTTATGTATTAGTATTAGAGTGATCTTTTGATTATTTTC
CTCACTATAAGGAAATTATTTCCTCAGGATGAGCTGCCATAACATTCCAC
TGTCTGATGGCAATTTTAAAGCCTGAAATTGAAGCCCATGGCTAGGCTAT
GAGAACCCTAGTTCGTATAGTAAAGTTGATATCTTCTGGATGTATACTAA
TTTTAGGCTTTATTTTAAAACTGCTGGAAACTGAAACTTAGACAAAAGTA
TTTTCAGGACATCATTTACAATGTTTAGCCCTAAAGAGTCAAGCTGTGGG
ATTCTGAGTCTTTCATATGTTACAGCAGAAACTTAAAAGCAAGAGGAAAT
TGGCTGGGCACAGTGGCTCTGTAATCCCAGCACTTTGGGAGGCTGAGGTG
GGTGGATCATGAGGTCAAGAGATTGAGACCATCCTAGCCAACATGGTGAA
ACCCCATCTCTACTAAAAATACAAAAATTAGCTGGGCGTGGTGGCACACG
CCTGTAATCCCAGCTAGTCAGGAGGCTGAGGCAGGAGAATATCTTGAACT
TGGGAGGCAGAGGTTGCAGTGAGCCAAGATTACATCACTGCACTCCAGCC
TGGTGACAGAGCGAGACTCCGACT
SEQ ID NO: 451
CTGAAACTGCACTGAACCCACAGGTAGGTTACATCACAGGACAGAAATCT
GAGGAGCTGGAGAAAGCAAAAGAATAAAGGATGGGCTGACACCAGAAGGA
ATTAAAGGAATTTTTATACTGAACTTCAATTACTTGTTCATTTGAAGTTT
GTTTTTTTAATGAACGTTTTTGCTGTTACTTAAATATAGTGTTTTGAAAG
TGTTTCAAATGTATTCAAGTTGGGATTTTCCATATTTTACTACAGTTCTG
TCTTAGTATGTTCACCATAAAACACTTATCATTAAAGCTCACAAAGTGCT
TTTTTGTAATATGAGGATAAAATGAAGCCATATAAGAATTTTTTTATATC
TGTACATTTAACCCACATTTGAGCTTTAGCCAAAATATATAGCTTTTTTT
TTTCTGACCTGGCCAACGTATTATCCAGCAAACATCAACTGAAGCAATAT
GGAAACACTTCCAAATGTTTGCCAATAATGCTATTAAGTGACTGATGTCA
ACATTAGTTACATGGCAAACTAAAGAGGCATTATACATTTTTAAAACACA
CTAACATATAACTGTAGATAATGTAAGGTTTATTTATATGCATATTTCAT
AGTATATTTAAATGTTTAAATATAAAAAAGGGTTTTTAAACACTTTTAAT
TTTTATCTTTGATTTTTTTTATTGATATCTCTTTCCAGGCTACTAATAAA
ATTGCCAGAACTAAACTATCAGGTAAAGGTTAAGGCATCAATTGACAAGT
AAGTTTTCTAATTTCGTTTTGAATTACAATTCCAAATGTAAGACTTTTAA
AAATGAATGGCCTTTATTTTATAGAATAATTTTGACCTTTTAAATTTACT
TATCTAACATTATATAATGAATGTACTTCAAATATTTGACTTTGAAGTCA
ACATTAACAAATTCATGGATCCTAATTAAAATTTACTATAAAACTGGAAT
CATTTATTACTTCCTT
SEQ ID NO: 452
TTTTTTTTTTTTTAAAAGAGATGGGTTCTCACTATGTTGCCCATAATGTT
TATGAGATTAAGTTCATCTTTTTTATCTGAGTAGTATTTTATTGTATGAA
TATACCACCATTTATTTATCTGTTGGTTATTTCCAGTTTTGGGCTATAAT
CCAAAATGCTTTTTTCAAACAATAGGCTATATATCATTAATGTCCGTTTA
TCAGCAGTATAAAATATCTTACCATAAATATTAATAAAAGAAGCATTCAT
ATATAAAATATAGATATTTCAAACCCTACAGAGGGCCTTTTAATGATTAA
ATATTTTGTCCTTACAAAAAGGTCCAGGTAATTACACCCATGAGGTTAAC
CTGCCTTAGTGCAGGACTTAAAATAAGGCTTCTCCTGCCATCTCTCTCCA
TTTGTAGAATGTGAAATTCTTTAAAATGCATCCTATATTAGGAATACTAT
AGCTGTGCACTGGTGTTTGTTCTCTTCTTTAAACTCGGGACCGTATATAT
CTGCTCAAATTGCCCAAGTATACATATGCTGCACTCCATCAAGTGTCAGG
CCACATTCTATCAGCACAGCGTGACTGCCTATCAGTGACAATATAAGTGA
GCTCTATTTGGATCCCTCTTACCCTACCTTTTATATTTATGACAGCATTA
TCATAAAACTCCAATATTCTTCAATAACTTACATGTTTGTTGTAGGATAA
AATTATTACCCTCAATGAACTACAT
SEQ ID NO: 453
ACCAGCTTCTTCACAGGTTCCACGAGTCATGTCAACACAGCGTGTTGCTA
ACACATCAACACAGACAATGGGTCCACGTCCTGCAGCTGCAGCCGCTGCA
GCTACTCCTGCTGTCCGCACCGTTCCACAGTATAAATATGCTGCAGGAGT
TCGCAATCCTCAGCAACATCTTAATGCACAGCCACAAGTTACAATGCAAC
AGCCTGCTGTTCATGTACAAGGTCAGGAACCTTTGACTGCTTCCATGTTG
GCATCTGCCCCTCCTCAAGAGCAAAAGCAAATGTTGGGTGAACGGCTGTT
TCCTCTTATTCAAGCCATGCACCCTACTCTTGCTGGTAAAATCACTGGCA
TGTTGTTGGAGATTGATAATTCAGAACTTCTTCATATGCTCGAGTCTCCA
GAGTCACTCCGTTCTAAGGTTGATGAAGCTGTAGCTGTACTACAAGCCCA
CCAAGCTAAAGAGGCTGCCCAGAAAGCAGTTAACAGTGCCACCGGTGTTC
CAACTGTTTAAAATTGATCAGGGACCATGAAAAGAAACTTGTGCTTCACC
GAAGAAAAATATCTAAACATCGAAAAACTTAAATATTATGGAAAAAAAAC
ATTGCAAAATATAAAATAAATAAAAAAAGGAAAGGAAACTTTGAACCTTA
TGTACCGAGCAAATGCCAGGTCTAGCAAACATAATGCTAGTCCTAGATTA
CTTATTGATTTAAAA
SEQ ID NO: 454
ACATTCTGGAAAAGGCAAAAGGGAGGAAGAACTGATTAGTGGTTAGCCCA
GGGTTAGAGTTGGGGAGAGGATATAATGAGGGAACTTTTGTGGATTCTGT
ACCATGATTATGATTACACAAACCTATGCATACATTGAAACACATAGAAC
TATACGTTGAAAAAAGTGAATCTGCCTGTATGTAAATTTAAAAGAAAAAT
ATTTTTTTAAAAAAACAGATGCTTCTTAACACATTATCATCTATGTCAGT
TTAACAGTTAGTAGACTTAGGCCAGGTGTCATGGCTCACTCCTGTAATCC
CAGTGCTTTGGGAGTCTGAGGTGGGACGATCTCTTGAGACTAGGAGGGAG
TTTGAGACAAACCTAGGCAATGTAATGAGACTCTTTCTCTACAAAAAATT
TTAAAGTTATCTGGACATGGTGGTGCCTGCCTGTAGTCCCAGCTACTTGG
GAGGCTGAGGTGGGAGGATTCCTTGAGCCCAGAAGTTCAAGGCTACAGTG
TGCTATGATAGAGCCACTGCACTCCAGCCTGGGCAACCAGGTGAGACCTT
GTCTCTAAAATGAATAAATAAAT
SEQ ID NO: 455
TGGTCTTTCACCCAGCCAGGGAGAAGGTTCTTCGCTCAGTATGAAGAAAA
GCAACCCAAAACTCTCAATCTGATTTGTTTTTGTTTATGTCGATGCCCTG
TAGTTTGAAAGTGAAGTAAAGATTTAGAATTCACCTAAGTCCAAAGGAAA
ACACGTGGTTTTTAAAGCCATTAGGTAAAAAAAGTTCTCAATAAAGGCAT
TACAATTTTTTAGGTTTAGAAAGATGGACTTTTCTGATAAATCTTGGCAG
ACATCTAAAAAAAAAACCATATTTTTCACAAGAAAATGCAAGTTACTTTT
TTTGGAAATAATACTCACTGATTATGGATAAAATGGAATATTTTCAGATA
CTATATTGGCTGTTTCAAAATAGTACTATTCTTTAAACTTGTAATTTTTG
CTAAGTTATTTGTCTTTGTTGTATCTATAAATATGTAAAAAATATTTAAA
TAGATGTACCTGTTTTGCTTTCACACTTAATAAAAAATTTTTTTTTGT
SEQ ID NO: 456
CGGGATCCCTAGTATAACACATTCAGTGTTCCCCTTTCAGTCTTACTACT
TTGACCGCGATGATGTGGCTTTGAAGAACTTTGCCAAATACTTTCTTCAC
CAATCTCATGAGGAGAGGGAACATGCTGAGAAACTGATGAAGCTGCAGAA
CCAACGAGGTGGCCGAATCTTCCTTCAGGATATCAAGAAACCAGACTGTG
ATGACTGGGAGAGCGGGCTGAATGCAATGGAGTGTGCATTACATTTGGAA
AAAATGTGAATCAGTCACTACTGGAACTGCACAAACTGGCCACTGACAAA
AATGACCCCCATGTGAGTATTGGAACCCCAGGAAATAAATGGAGGAAATC
ATTTGCCTTAGGGATTGGGAAAGCTGCCCACTAACTGTCTTCCCCATTGT
TTTGCAGTTGTGTGACTTCATTGAGACACATTACCTGAATGAGCAGGTGA
AAGCCATCAAAGAATTGGGTGACCACGTGACCAACTTGCGCAAGATGGGA
GCGCCCGAATCTGGCTTGGCGGAATATCTCTTTGACAAGCACACCCTGGG
AGACAGTGATAATGAAAGCTAAGCCTCGGGCTAATTTCCCCATAGCCGTG
GGGTGACTTCCCTGGTCACCAAGGCAGTGCATGCATGTTGGGGTTTCCTT
TACCTTTTCTATAAGTTGTACCAAAACATCCACTTAAGTTCTTTGATTTG
TACCATTCCTTCAAATAAAGAAATTTGGTACC
SEQ ID NO: 457
TGCGCAGACCAGACTTCGCTCGTACTCGTGCGCCTCGCTTCGCTTTTCCT
CCGCAACCATGTCTGACAAACCCGATATGGCTGAGATCGAGAAATTCGAT
AAGTCGAAACTGAAGAAGACAGAGACGCAAGAGAAAAATCCACTGCCTTC
CAAAGAAACGATTGAACAGGAGAAGCAAGCAGGCGAATCGTAATGAGGCG
TGCGCCGCCAATATGCACTGTACATTCCACAAGCATTGCCTTCTTATTTT
ACTTCTTTTAGCTGTTTAACTTTGTAAGATGCAAAGAGGTTGGATCAAGT
TTAAATGACTGTGCTGCCCCTTTCACATCAAAGAACTACTGACAACGAAG
GCCGCGCCTGCCTTTCCCATCTGTCTATCTATCTGGCTGGCAGGGAAGGA
AAGAACTTGCATGTTGGTGAAGGAAGAAGTGGGGTGGAAGAAGTGGGGTG
GGACGACAGTGAAAT
SEQ ID NO: 458
TATAAATACACTCCGGGATGATTTACCCCCGGAGGTCAGCTAGTAAAATA
CATGAGTAGAATTCCTTAAAGTATGTGATAATTGCTCATCACTATCCAAG
TGTGACATAAATCATAAAAAGAATTGACAAAATCAGGGTCGCAAAGAGAA
TTGAAAAAAATCTGTCACAACCAAAATTTAAATTGACCTCTGTCCTAGAG
TATGAGAGCCACACTGAACAGAAAAACCAGATAAATCTTTTATAAAATAT
TCATTTGCAGCCCCATTAACGTTGCTTGTCACCCCACCTCCCCATGTCCT
TGGACAAACTGAATGTATAGTAACATCATCCCAGGCCAGGCGCGGTGGCT
CATGCCTGTAATCCCAGCACTTTGTGAGGCTAAGGCAGGCAGATCAGGAG
GTCAGGAGTTCAGGACCAGCCTGGCCAAAAAGGTGAAACTCCGTCTCTAC
TAACAATACAAAAATTAGCTGGGTGCGGTAGTAGGCGCCTGTAATCCCAG
CTACTCGGGAGGCTGAGGCAGGAGAATTGCTCAAACCCGGAAGGTGGAGG
TTGCAGTGAGCTGAGATCGTGCCACTGCACTCCAGCCTGGGTGACAGAGC
AAGACTCTGTCTCGGGGAGGGGGGTGGCGGAGATAAAGAAATAACATCAT
CTTATACTGTCAAGCTCAAGGTGTCTGCAGCCTTATCTTCAGGGGAAGTT
GTGTCTTTCTCAGGGAAGATACAGATTTCAATTTAGAGCAAGACAGAGAG
AAGTTACATTCAGAGAGGAAAATGCAGTAGTCTAACTG
SEQ ID NO: 459
GCGGCCGCGCTCTTTTCAATTTTTAAAAAGAAGTTTGTTTTCCATTTCAG
TAATTTCTGCTTTGATCTTCCTTATGTCCTCCTATTGAGTTGATCAGCTT
TCTTTATTCTTGCCTTTTCTCCTCTGTGTGCCCTTTCTATTAACGTATTT
ACCCTTAGGCTGGGCACAATGGCTGATGCCTGTAATCCCTGCACTTTGGG
AGGCCGAGGCAGGTGGATCACCTAAGGTCAGGAGTTCAAGACCAGCCTGG
CCAACATGGTGAAACCTGGTCTCTACTAAAAACACAAAAATTAGCCAGGC
ATGGTGGTGTGCACCTGTAATCCCAGCTACTCAGGAGGCTGAGGCAGGAG
AATTGCTTGAACCTGGGAGGCGGAGATTGTGCCAAAGCACTCCAGCCTGG
GCAACAAAATGAGACTTTGTGTC
SEQ ID NO: 460
CACCAGGCTGTCTTCAGATACTTCATACAGAAATGAGCCTCCCTGTGGGG
TCCTCTTCCCTCCTTCAGCCTGTCCATCAACACAGCATTGCGGGATCCTT
ACCATGGCATCCAGCCCTGGAGATGCTTCAGGAAAGTTGCAGGTCCATGC
TGCAGGACAGGCTCAGATCAGCAGAGACGCATCTCACATCGGGCTGTGAA
ATTCAAGTTGAGCTGCAATTGGCAATGAGAA
SEQ ID NO: 461
GTTATTCACTGAGACCGTGCCCCGGTTATGAGGTTGTACCAGAAAGCAAG
TATTCACTATGCACACTATTCACCGCTCACCCTAGCATTGAAGCCAGCCT
GTAGCCTGAAAGCCTTTGCTTTGAGGGCAGGTCTTTCCCCAAAATGCAGA
CACGAAGGTGCAAAGTGAAGCTGCCAGTCTTGCAAAAGATGTAACTTGTC
ACGAAGGCCACGAGTGGCAGGGAGAGCTGTCCCACATTTGCGGAAGTGGC
TATGTGAGGACGGGGGAGGCGGGTCCCTTAGAGATGAGACAATCATAAGG
GGAGATATCAGAGAAAATCGTAAGGGGAGCAGATGGTTGTCAAGAGAATA
GGCTGACCATCGAAGGACTGGCAGAAGCTTTCAGAAAACCACTGGACGGC
TGGGCACAGTGGCTTAGGCCTGTAATCCCAGCACTTTGGGAGGCTGACGC
AGGTGAATCACTTGAGGTCAGGAGTTCCAGACCAGCCTGGCCAACATGGT
GAAACCCCATCTCTACAGAAAATATAAAAATTAGCCAGGCGTGGTGGCAC
AAGCCTAGAATCCCAGCTACTTGGGAGGCTGAGGCAGGCGAATGGCTTGA
ACCCAGGAGTCAGAGGCTGCAGTGAGTCGAGATTGTTCCACTGCACTCCA
GCCTGGGTGACAGTGCAAGACTCCTTCCAAAAAAAAA
SEQ ID NO: 462
TTCGTGAGTGATGGCGTCCCGGGTTGCTTGCCGGTGCTGGCCGCCGCCGG
GAGAGCCCGGGGCAGAGCAGAGGTGCTCATCAGCACTGTAGGCCCGGAAG
ATTGTGTGGTCCCGTTCCTGACCCGGCCTAAGGTCCCTGTCTTGCAGCTG
GATAGCGGCAACTACCTCTTCTCCACTAGTGCAATCTGCCGATATTTTTT
TTTGTTATCTGGCTGGGAGCAAGATGACCTCACTAACCAGTGGCTGGAAT
GGGAAGCGACAGAGCTGCAGCCAGCTTTGTCTGCTGCCCTGTACTATTTA
GTGGTCCAAGGCAAGAAGGGGGAAGATGTTCTTGGTTCAGTGCGGAGAGC
CCTGACTCACATTGACCACAGCTTGAGTCGTCAGAACTGTCCTTTCCTGG
CTGGGGAGACAGAATCTCTAGCCGACATTGTTTTGTGGGGAGCCCTATAC
CCATTACTGCAAGATCCCGCCTACCTCCCTGAGGAGCTGAGTGCCCTGCA
CAGCTGGTTCCAGACACTGAGTACCCAGGAACCATGTCAGCGAGCTGCAG
AGACTGTACTGAAACAGCAAGGTGTCCTGGCTCTCCGGCCTTACCTCCAA
AAGCAGCCCCAGCCCAGCCCCGCTGAGGGAAGGGCTGTCACCAATGAGCC
TGAGGAGGAGGAGCTGGCTACCCTATCTGAGGAGGAGATTGCTATGGCTG
TTACTGCTTGGGAGAANGGCCTAGAAAGTTTTGCCCCCGCTGCGGCCCCA
GCANAATCCAGTGTTGCCTGTGGCTGGAGAAAGGAATGTGCTCATCACCA
GTGCCCTCCNTTACGTCAACAATGTCCCCCACCTTGGGAACATCATTGGT
TGTGTGCTCAGTGCCCGATGTCTT
SEQ ID NO: 463
CAGTGAGCCAAGATCACACCACTGCACTCCAGCCTGGACAACAGAACGAG
ACTCCATATCAAAAAAATTAAATTAAAATATAATAAATTTCTTGCCGGGC
GCAGTGGCTCACACCTGTAATCCCAGCACTTTGGGAGGCCGAGGTGGGCG
GATCACGAAGTCAGGAGATTGAGACCATCCTGGCTAATACAGTGAAACCC
CGTCTCTACTATAAATACAAAAAATTAGCTGGGCATGGTGGCGGGCGTCT
GTAGTCCCAGCTACTCAGGAGTCTGAGGCAGGAGAATGGTGTGAACCCGG
GAGGCGGAGCTTGCAGTGAGCCGAGATCGTGCCACTGCAATCCAGCCTGG
GCAGCAGAACGAGACTCCATCTCAAATAAATAAATAAATAAAATGAATTT
CAGCTAGAAGAGCCTTATTCCATTTTCCTTTTTATTAAACATCTGGCATA
AGTTGGTAAGTATGTGAAGTTTATCATATATTCTTATGCGAATTATTATT
TTCGCCTTTTTTTTTATAATTCTGTCTGGGATTTGAATAGTAGAGTTTGA
ATTCAGGAAGGACACCTGTGATAGGACAATAAAAT
SEQ ID NO: 464
CTGATTGCAAAAACATTACAACTCAGTACTGCGGCTTTCATTCAAATAGG
TTATATGTATAAACTGAGGTTCAACAATATTGTATTTGAGATGGGAAAGT
TAAAGAAATGCAATAATGTAAATAATACTTAAGAAAATAAGATCTCAGGA
AACTGTGTATACTCTGTACTTTTATGCAACTTTATCAGATCATTTCAGTA
TATGCATCAAGGATATAGTGTATATGACATGAACTTTGAGTGCAAAAACT
GTACTATGTACCTTTTGTTTATTTTGCTGTCAACATCTAAATAAAGGTTT
TTTTG
SEQ ID NO: 465
CGAAAGGACTACAGAGCCCCGAATTAATACCAATAGAAGGGCAATGCTTT
TAGATTAAAATGAAGGTGACTTAAACAGCTTAAAGTTTAGTTTAAAAGTT
GTAGGTGATTAAAATAATTTGAAGGCGATCTTTTAAAAAGAGATTAAACC
GAAGGTGATTAAAAGACCTTGAAATCCATGACGCAGGGAGAATTGCGTCA
TTTAAAGCCTAGTTAACGCATTTACTAAACGCAGACGAAAATGGAAAGAT
TAATTGGGAGTGGTAGGATGAAACAATTTGGAGAAGATAGAAGTTTGAAG
TGGAAAACTGGAAGACAGAAGTACGGGAAGGCGAAGAAAAGAATAGATAA
GATAGGGAAATTAGAAGATAAAAACATACTTTTAGAAGAAAAAAGATAAA
TTTAAACCTGAAAAGTAGGAAG
SEQ ID NO: 466
GTCCAGNAGAAAGTTCAGTGACTTGTCCAGAGCTGCAGGTCTTAAGAGGC
TGAAATCTCGCCTCTGCCTCGAGGCTGCGGTTCCACTGACCCATACTACT
TGCCTTCAGGAAAGAGAAATGGTGTAGGAAGGCTGTGGATGAAGACGCTT
ACATTCATGAAGGATTTGGATAGGCGAACATGAGCTTTTCCACCAAATTT
CAGAATTTTAAGAAATGCCTTAAATTATTTCTTAAAAATCAATTTGGGGC
AGACGAGAAGTTCTGATAATAGTTTTTAGGGAACATGATAAAATTCTGAC
CTTAGAAGTGGTATACCAGTTTGAGAAGAAGAACAAGCTATAAACGGTGT
AGATAACATTCACGGCTATTTAAGAAAGAGTTACTAAGGGAAACCAGAAT
GACTTAAGAGTGTTACTCTTCTTTTTCTGAGAGAACAATAGCATCATCTC
AGAAAGCCTTTCATGCCATTAATAGGTAAGAATCTGGGCTTCTTGGACCA
TGGGTTAGACTTTCTTACAAAACCATAATATGCATTTCCTAGCAAAATTT
ATGCTATTACATTTCCTTATCTCAACAAAGACTGGTAAATTCAGTACTTA
TTCCTCAATTTTCCTACCCTTAAAATGGGGATATTCTGCCTCTCCAAGGA
ATGCTGGGAACAAGCAAGTCCTCATGTTAGGGGTCTTTGAGTTTTCATGG
AAGTTTAGGTTATTTATATGATGACATAGTTGTCAACTTACTTTCAGGAT
GGACTTTTCTTTTGTGAGTTTGTGACCTAAATACAATAGTTGTTATGCAT
GTCCAGTTTATGGAAGTACCACTGCAATANCAG
SEQ ID NO: 467
CAGTGCAGCCAAGTATCACACCACTGCACTCCAGTCCTGGACAACAGAAA
CGANTACTCCATATCAAAAAAATTAAATTAAANGATAATAAATTTCTTGC
CGGGCGCAGTGGCTCACACCTGTAATCCCAGCACTTTGGGAGGCCGAGGT
GGGCGGATCACGAAGTCAGGAGATTGAGACCATCCTGGCTAATACAGTGA
AATCCCCGTCTCTACTATAAATACAAAAAATTAGCTGGGCATGGTGGCGG
GCGTCTGTAGTCCCAGCTACTCAGGAGTCTGAGGCAGGAGAATGGTGTGA
ACCCGGGAGGCGGAGCTTGCAGTGAGCCGAGATCGTGCCACTGCAATCCA
GCCTGGGCAGCAGAACGAGACTCCATCTCAAATAAATAAATAAATAAAAT
GAATTTCAGCTAGAAGAGCCTTATTCCATTTTCCTTTTTATTAAACATCT
GGCATAAGTTGGTAAGTATGTGAAGTTTATCATATATTCTTATGCGAATT
ATTATTTTCGCCTTTTTTTTTATAATTCTGTCTGGGATTTGAATAGTAGA
GTTTGAATTCAGGAAGGACACCTGTGATAGGACAATAAAATCTA
SEQ ID NO: 468
GAAAGCACATATGATATACATGTGTGTCATATGTATTATTTTGTTTGCCA
TCTGAGTCTTCAAAATTTGTTACAGAATACCTGCATATTAATATTTCAAG
GTATGGATTAAT
SEQ ID NO: 469
CTGAGTATTAACTAAAAAAAAAAAAAAAAAAAAAAAAAAA
SEQ ID NO: 470
CCAAACCCAACTGGTCCAGTAGGATACTCACCTTACAGGGGGCGTCTCAA
GAGTCTCACAGTTCCCTTGGGTCTTAAGAGACTCACTGTTGGACCAGGCG
TGGTGACTCACGCCTGTAAAACCAGCACTTTGGGAGGCCGAGGCGGGCGG
ATCAGTTGAGGTCAAGAGTTCAAGACCAGCCTGACCAAGGTGCTGAAACC
CCGTCTCTACTAAAAATACAAAAATTAGCCAGGCATGGTGGTGTGCGCCT
GTAATCCCAGCTACTCCAGAGGCTGAGGCAGGAGAATCTCTTGAACCCAG
GAGGTGGAGGTTGCAGTGAGTCGAGATCATGCCACTGCACTCCAGCCTGG
GTGACAGAGCGAGACTCCGTCTTAGAAAAAAAAAAAAAAAAAAAAAGAAC
CTCACAGTTCAGCAGGGTTCTAGCATGAGACAATGAGGACAAGGGTAGGT
GAGCAGGTGGAAAGAGTGAGAACAGGTCAATTGTGATGGAGAAAATAATA
AAGACAGAAAAGGCAGAAGACTGCCTGGCAGAAGACCTGTCCCAGCAGAT
ACAAAAATACAGACAACAGGAGCCAGCATAGACCCTTGACCTGTGTAAGT
CTTTCTCAGGCCTTCTTTTAAGTAGAAACATGCCTTTGAAAAAAAGTTTT
AATAAACAGGAAAATCATAAATCCCTATTTACATAAATAATATATCCTGG
TCTTATTCTTAAAACCATTGATTTTTCACGGCTCATTAANAAAGCTGGGC
GAGGTGGCTCACGCCCGTCATCCTAGCACTTTGGGAGGCCGAGGCGGGCA
NATCACAAGGTGAGGAGTTGGGAGACCAGCCTGACCAACACGGTGAAACC
CAGTCTCTACTAAAAATACAAAAATTANCTGGGGGTGGTGGTGTGTGCCT
GTAATCCAAGCTACTCGGGAGGCTGAGGCAGGA
SEQ ID NO: 471
CTTACTACCTCCAACATGAAACAAGCAGCCCCGCACTTCTCGAAGGTCTG
AGTTACTTGGAATCGTTTTACCACATGATGGACAGAAGGAATATTTCAGA
TATCTCTGAAAACCTCAAGCGTTACCTTCTTCAGTATTTTAAGCCAGTGA
TTGACAGGCAAAGCTGGAGTGACAAGGGCTCAGTCTGGGACAGGATGCTC
CGCTCGGCTCTCTTGAAGCTGGCCTGTGACCTGAACCATGCTCCTTGCAT
CCAGAAAGCTGCTGAACTCTTCTCCCAGTGGATGGAATCCAGTGGAAAAT
TAAATATACCAACAGATGTTTTAAAGATTGTGTATTCTGTGGGTGCTCAG
ACAACAGCAGGATGGAATTACCTTTTAGAGCAATATGAACTGTCAATGTC
AAGTGCTGAACAAAACAAAATTCTGTATGCTTTGTCAACGAGCAAGCATC
AGGAAAAGTTACTGAAGTTAATTGAACTAGGAATGGAAGGAAAGGTTATC
AAGACACAGAACTTGGCAGCTCTCCTTCATGCGATTGCCAGACGTCCAAA
GGGGCAGCAACTAGCATGGGATTTTGTAAGAGAAAATTGGACCCATCTTC
TGAAAAAATTTGACTTGGGCTCATATGACATAAGGATGATCATCTCTGGC
ACAACAGCTCACTTTTCTTCCAAGGATAAGTTGCAAGAGGTGAAACTATT
TTTTGAATCTCTTGAGGCTCAAGGATCACATCTGGATATTTTTCAAACTG
TTCTGGAAACGATAACCAAAAATATAAAATGGCTGGAGAAGAATCTTCCG
ACTCTGAGGACTTGGCTAATGGTTAATACTTAAATGGTCAATAGAAAAAG
TAGGCTGGGCGCGGTGGCTCACGCCTGTAATCCCAGCACTTTGGGA
SEQ ID NO: 472
AAAATTTTTTTTTTTTTTTTTTTTTTTTTTTTTTTTTTTTTTTTTTTTTT
TTTTTTTTTTTTTTTTTTTTTTTTTTTTTTTTTTTTTTTTTTTTTTTTTT
TTTTTTTTCAGTGTTAAAGTAGGTTTGTCGACGCGGCCACGAATTTCCCG
GGGACCAA
SEQ ID NO: 473
TTTTTTTTTTTTTTTGGGAGTCAGTTTTCTTTTCTTTTCTTTCTTTTTTT
TTTTTTGNTTTTCGGAAACGGAGTCTCGCTTTCTCGCCCACTCTGGAGTG
GNGCAGTGGGGNGGTCTCAGCTCACCACAGCCTCCACCTCCTGGGCCCAA
GCGATCCTNTCACCTCAGCCTCCTGCGTAGCTGGGACTACAGGCGTGCAC
CACCATTCCCAGGTAATTTTTGTATTTTTTGTANANACAGGGTTTCACTG
TTGTTGCCCAGGCTGGTCTCGAACTCCTGCTTCAGTCTGCCANAATGCTG
GATTCTAGGCGTGAGCCACCGNGCCTGGCCCAAAAGTTACTTTTCTTACA
GAAGCAAAGCTTTAATGCATTTTACTGAATGCTTATAGCTTTGTAGATAC
TGAAAAGAGTATGAGCGTCACATACAGACACATNTAACAGCACTGCCTCC
AACCAGCCCCTACCCACTGGTCAGGNGAGTAANAATCAAAATTCTTTTCT
GNGAGTGGAACGGAAATTTCATCTCTCCTCCTCAGGCAAGTAGTTAANAG
GCTGGNGGGAGTCATGGCCCCATTTTGTTCAAAATACAAGCTCCACAGGA
ACAAAAGGCTGAACTGCTCACCTCCCAACTGATGAACCTCGTCTTTGTTC
CATGTCAAAGGGGCCTTTGTGTTACTGCAGCAGAAACTCCAGCTATCAAA
CCATCAGGCACCAAAAGTAAAACTCCTTTCTCTAAAAAGACCTCTCTTTA
CCTGAGCCTTTCAATGCATCTTTGCCCCCANATAATCCTGGATGAGATAA
TCCCCAGAGGAANACCAGCGCTTGCCTAGTGAAATTATACTATGAGACAA
GGGTAAAAGACCTCAAANACCGGGTTGGCAGGTAAGGGAGTAGGGN
SEQ ID NO: 474
TCNGTGGCACCCGTTTCCGGCACCTTCAGACTCTGAAGAGCCACCTGCGA
ATCCACACAGGAGAGAAACCTTACCATGTACGTAAGCCTCTTGAGGCCGC
TCTCTGACCTGCGGGGATGTGGAGGGCAGGGAAGGAGGTGGAGCGCAGGG
AAGGAGGTGGAGCAGGGAGGCAGTGGAACTGTTTGCTCCCATCTCAAGCA
CACAGTGGGGCAACCACTACGCTAATGGTTGGAAGACCTAGATCTGGGCC
CAATGGCCAGACACCCTGCTTGACCTTGGCCCAAGCATTAGGGGACTCAT
CTTTAAAATGAGGGTATGGGACTAGATGATCTGGGCCTTAGGAGAGGAGT
SEQ ID NO: 475
CGGCTNCTACCCTGCGGAGATCACACTGACCTGGCAGTGGGATGGGGAGG
ACCAAACTCAGGACACCGAGCTTGTGGAGACCAGGCCAGCAGGAGATGGA
ACCTTCCAGAAGTGGGCAGCTGTGGTGGTGCCTTCTGGAGAAGAGCAGAG
ATACACGTGCCATGTTCAGCACGAGGGGCTGCCGGAGCCCCTCACCCTGA
GATGGAAGCCGTCTTCCCAGCCCACCATCCCCATCGTGGGCATCGTTGCT
GGCCTGGCTGTCCTGGCTGTCCTAGCTGTCCTAGGAGCTATGGTGGCTGT
TGTGATGTGTAGGAGGAAGAGCTCAGGTGGAAAAGGAGGGAGCTGCTCTC
AGGCTGCGTCCAGCAACAGTGCCCAGGGCTCTGATGAGTCTCTCATCGCT
TGTAAAGCCTGAGACAGCTGCCTGTGTGGGACTGAGATGCAGGATTTCTT
CACACCTCTCCTTTGTGACTTCAAGAGCCTCTGGCATCTCTTTCTGCAAA
GGCATCTGAATGTGTCTGCGTTCCTGTTAGCATAATGTGAGGAGGTGGAG
AGACAGCCCACCCCCGTGTCCACCGTGACCCCTGTCCCCACACTGACCTG
TGTTCCCTCCCCGATCATCTTTCCTGTTCCAGAGAAGTGGGCTGGATGTC
TCCATCTCTGTCTCAACTTCATGGTGCGCTGAGCTGCAACTTCTTACTTC
CCTAATGAAGTTAAGAACCTGAATATAAATTTGTTTTCTCAAATATTTGC
TATGAAGGGTTGATGGATTAATTAAATAAGTCAATTCCTGGAAGTTGAGA
GAGCAAATAAAGACCTGAGAACCTTCCANAATCCG
SEQ ID NO: 476
TGAAACAAAATGAATTTNTATGGGTAAGAGAGGGTAATATTTTAGAGTTG
TGTTACAAAACTACAAATTTTTATTAAATTAATAAATCAGAATACTAAAT
CCATGTGTTTTTTTCTTTCTTAAAAAATATCTTTTGGCTGGGCACGGTAG
CTCATGGCTGTAATCCCAGCACTTTGGGAGGCTGAGGTGGGTGGATCGCC
TGATGTCAGGAGTTCAAGACCAGCCTGGTCAACATGTTGAAACCCCATCT
CTACTAAAAATATAAAAATTAGCCGGTGTGGTGGTGGGCGCCTGTAATCC
CAGCTACTCAGGAGGCTAAGGCAGGAGAATTGCGTGAACCCAGGAGTTCA
GTGATGTAGCGGGGAGCTGAGATTGTGCCACTACACTCCAGCCTGGATGA
CAGAGTGAGACTCCATCTCAAAAAAAAAAAAAAAAAA
SEQ ID NO: 477
GCATAATGTGAGGAGGTGGAGAGACAGCCCACCCCCGTGTCCACCGTGAC
CCCTGTTCCCATGCTGACTTGTGTTTCCTCCCCAGTCATCTTTCCTGTTC
CAGAGAGGTGGGGCTGGATGTCTCCATCTCTGTCTCAACTTTATGTGCAC
TGAGCTGCAACTTCTTACTTCCCTACTGAAAATAAGAATCTGAATATAAA
TTTGTTTTCTCAAATATTTGCTATGAGAGGTTGATGGATTAATTAAATAA
GTCAATTCCTGGAATTTGAGAGAGCAAATAAAGACCTGAGAACCTTCCAG
AAAAAAAAAAAAAAAAAAAAAAAAAAAAAAAAAAAAAAAAAAAAAAAAAA
AAAAAAAAAAAAAAAAAAA
SEQ ID NO: 478
CTTACCATGTCAGTGCACAGAAATGCTGTCTTGGGATGTAGGAAAAATAA
ATCCACAAAAGCTACCAAGTTTGAAGGGGACCATGAGTCTTCAGGCTGGA
GCTTCCAAACCAGATGAAAACCCCACAATTAACCTGCAGTTTAAGATCCA
GCAGCTGGCCATTTCTGGACTCAAGGTGAATCGTCTGGATATGTATGGAG
AAAAGTACAAACCCTTTAAGGGCATAAAATACATGACCAAAGCTGGGAAG
TTCCAAGTTCGAACCTGAAGGGAGCATTTGCTGAGGGAATAGTCTTGCAC
ATTTTTTCATTTCTTACTTGTCTAAAAGTAAAAAAAAATATCAGCCTGTC
TCCTAGGTCAGTCCCCTCCTGGACCCACCCGCTCCCTTTTTTCCTTAGCC
TTCAGTGCCATGGAACTAATCAAGGGAGGAAAAGGTCACCAGGGAGAACT
GGACAGAACTGAAACACAGCAACACCAGTTCTCAAGGACAAGGTGTGTGA
TGGGGGTAGGAAGCTTGGTGCTTATGTAACCATTTTAAACGTGGTTTCTA
TAGGAAAGACCAACATTTGTTTAGCTTGCTTGGCTTTAATTATCTAAAGC
CAATGAAAGACTTCTTTGTTGATTTTTTAAGATAGAAAGATT
SEQ ID NO: 479
CAAACACTATGTTATTTTATGAANAAGACTTGAACATCTATGGATTTTGG
TATTTGCAAGGGGTGAATGGGGTATTTGCAAGCAGTGAATGAGGAGGCCT
GGAACCAATCTTCTGCTGATATTGAGGCACAACTGAAAAAGGTATATTAC
TTAAATCTCTTATTGTATTGTAAACTGTATAAGTAATGAAATTAAAAGGC
AGAAATTGTCAGACTGAATAAAATGAAAAGACCAAACAATATGCTGCTTA
CAAGAAACACAATTCAAATATAAGGACACAATTAGTTTAAAGGAAAAGAA
CTGGAAAAGATATACCATGATAACACAAGTCAGAAGAAAGCTGCTGTGGA
TATATTAATATGAGATGTAGATTTCAGAGCAGTGAATATTGCCAGGCATA
AAGAAAGTTATTACATAATAATTAAGGTATCAGTTCATCAAGAAGATGTA
ATAACCCTAAGTATTTATACAACTAATATCAGAGCTTCAAAATACATGAA
GCAAAAACCAGTGGAATTGATAGGAGAAACACACAATTACACAATTATAG
TCAGAATTTTCAACATATCTTTCTCAATGGAGAAAACAACTAGACAGGAA
ATCATTAAGGATATAGATGATTTAAATTATATGATCAACTACCTGGACGT
AATTGGCATTTATGGAACACTGCACCACCAACAGCAGAGTACATATTATT
TTCAAGTACACAGAAAACAGTTACCAATATAGACCATTTTCTGGGTCATA
AAACACATCTCAATAAATGTAAAACAATTAATGTTATATAAAGTATGTGC
TCTGACCNCAAAGGAATTAGAGATCAATAAAAGAACATCTTTGAAAAATC
TCACNTATTTAAAAACTAATAACTCACTTCTAAATAACTCCTGTNTCAAG
AGAATNAAANGG
SEQ ID NO: 480
CCCAGCCTCACTGCGCCCCGTCAGGCCAGGCAGCTGCCCTCAGGGTCTGC
CAAGGTGGGGGTCAAGGGCCATGGGGGCAGGTAGCTCTGCCTGCAAAGCC
CACAAGCATGTCAGATCACCTGGGCTGCAGACAGACAAACACCTGAGCTG
TTCTGAATACCTTCAGGTTCCTGGCCTCGCTGAGCAAGTGCAGAAATTTT
TACCTTCAAGGATCAGGGTTTTTCTGTTTGTTTGTTTTTTAACACACACA
TATGTGAACAAAGAGTATGCGTTTGTACTGGCAGAAGAAGCGTCTGGTAA
GACAACCAGCAAGTTAACAATGGTCACCTCCAGAAATGGGCTGGGTAAAC
CAAAGAATTTTTTTGTTTTTGTTTTTTTTGAGTCAGGGTCTAGCTCTGTC
ACCCAGGCTGGAACGCACTGGTGTGATCACGGCTCACTGCAGCCTTGACC
TCCCTGGCTCAAGCAATCCTCCCAGCTCAGCCTCCTGAGTCGTTGGGACT
ACAGGCACGTGCCACCACGCCTGACACATTTTTTAAATTTTTGTAGAGAC
AGTGTTTCACCATGTTGCCCAGGCAGGTCTCAAACTCCTGGGCTCAAGTG
GTCCTCCAGCTTCAGCCTCCCAAAGTGCTAGGATTATAGGTGTGAGCCAC
AGTGCCCAGCCCCGTAGTGGAGAATTTCTGTTGAATGAACCAAAAGCAAC
TGCCAACCTCTCCATGCACCATGTGTTTCAGAGGAGAAAGCACAGTGAAG
AATGCAGTGTGTTCTGAGGTCCTGTCACCCCTGAGGCTGTGTGTGTCCTT
TGCCAAATTAAAGAGTCTTACTGAATGCGGTGCATCCAGGAGACAGGCCN
AGGTTTGGACTGGTAAAAAAAAA
SEQ ID NO: 481
CAGACACCTGGNAGAACGGGAAGGAGACGCTGCAGCGCGCGGACCCCCCA
AAGACACATGTGACCCACCACCCCATCTNTGACCATGAGGCCACCCTGAG
GTGCTGGGCCCTGGGCTTCTACCCTGCGGAGATCACACTGACCTGGCAGC
GGGATGGCGAGGACCAAACTCAGGACACCGAGCTTGTGGAGACCAGACCA
GCAGGAGACAGAACCTTCCAGAAGTGGGCAGCTGTGGTGGTGCCTTCTGG
AGAAGAGCAGAGATACACATGCCATGTACAGCATGAGGGGCTGCCGAAGC
CCCTCACCCTGAGATGGGAGCCATCTTCCCAGTCCACCGTCCCCATCGTG
GGCATTGTTGCTGGCCTGGCTGTCCTAGCAGTTGTGGTCATCGGAGCTGT
GGTCGCTGCTGTGATGTGTAGGAGGAAGAGTTCAGGTGGAAAAGGAGGGA
GCTACTCTCAGGCTGCGTCCAGCGACAGTGCCCAGGGCTCTGATGTGTCT
CTCACAGCTTGAAAAGCCTGAGACAGCTGTNTTGTGAGGGACTGAGATGC
AGGATTTCTTCACGCCTCCCCTTTGTGACTTCAAGAGCCTCTGGCATCTC
TTTCTGCAAAGGCACCTGAATGTGTCTGCGTCCTTGTTAGCATAATGTGA
GGAGGTGGAGAGACAGCCCACCCTTGTGTCAACTGTGACCCCCTGTTCCC
ATGCTGACCTGTGTTTCCTCCCCAGTCATCTTTTTTGTTCNCAATAGGTG
GGGCCTGGATGTCTCCATCTCTGTNTCA
SEQ ID NO: 482
TTATAAGGTACTTTTAAGGTATTTTAGTTGTCTTAGTCTATATTTCTGTA
CTCACCTTTCTTTATCCACTCATCAGTTGATGGGCATGTAGGTTGGTTCC
ATATCTTTGCAATTCTGAATTGTGCTGTGATCAGGTGTCTTTTTAGTATA
ATGATTTACTCTCCTTTGGGTAGATACCCAGTAGTGGGATTGCTGGATCG
AATGGTTTTTATAATTTTCTATTTTACCACAGTTTCTCTCTGCATTTTTC
CTCTTTGACCACTAACCATGTGAAATTCTCATATTGACCTTTATAATGAT
CATGAACTCTTAGTATCATTGGGAAGGCCACATTTGCCACTTATGATTGT
AAACCTTATCCTCCATTTTTCCTGTTATTGTTGGTGCAAAAAGCACCTAT
TATACCAGGACTTTAAAAATCAGTCTGATAAGTCTTTGATAAGTCTAATA
ATAATAACTGATAAGTCCATTGAATTTGCTTCTGATTACTTTTTCTTTAG
TAGCTAAACATGTATGTACTCCTATGATTACAATGAACACTCCTCTCCAT
TTAAATTAATTATTTACATTGATGAAATAGCAAAATGTTAATGACTAAAT
ACTGTCTTGGTTTTTTCGTTCCAGGTCAGTCAATATTAACTTCTTATAAT
TTTCTTTTTTTTCTTT
SEQ ID NO: 483
GCAAGGACTAACCCCTATACCTTCTGCATAATGAATTAACTAGAAATAAC
TTTGCAAGGAGAGCCAAAGCTAAGACCCCCGAAACCAGACGAGCTACCTA
AGAACAGCTAAAAGAGCACACCCGTCTATGTAGCAAAATAGTGGGAAGAT
TTATAGGTAGAGGCGACAAACCTACCGAGCCTGGTGATAGCTGGTTGTCC
AAGATAGAATCTTAGTTCAACTTTAAATTTGCCCACAGAACCCTCTAAAT
CCCCTTGTAAATTTAACTGTTAGTCCAAAGAGGAACAGCTCTTTGGACAC
TAGGAAAAAACCTTGTAGAGAGAGTAAAAAATTTAACACCCATAGTAGGC
CTAAAAGCAGCCACCAATTAAGAAAGCGTTCAAGCTCAACACCCACTACC
TAAAAAATCCCAAACATATAACTGAACTCCTCACACCCAATTGGACCAAT
CTATCACCCTATAGAAGAACTAATGTTAGTATAAGTAACATGAAAACATT
CTCCTCCGCATAAGCCTGCGTCAGATTAAAACACTGAACTGACAATTAAC
AGCCCAATATCTACAATCAACCAACAAGTCATTATTACCCTCACTGTCAA
CCCAACACAGGCATGCTCATAAGGAAAGGT
SEQ ID NO: 484
GGCCACCGGGTGCAAGGTCAGGGCTGGGGTGGAGGCTGGGAAGCCCAGGG
CTTGGCCCACTGTGGCCGCCTTGTGTGGTCACTGCTTTCCTGGGCCTGCT
GTGAGCTCCCTCTAGGACCCCAGGCCTGTCTGGTGGGTCACTGTGACCAC
CACCTTGCACAGCACCTGGCGCGTGGCAGGTGCTCAAACATTACTTGTTT
CGGAATGAACTTCATCTTGCTCTTGGCTTTTTGACTAATGCTGTGGAACA
TCTGACTAATTAGTGACTCTTTGGGGCCCCCAGTTTCCCAGCTATAAAGT
GGTAATATTAAGATAATAATTCGGCCGGGCGCGGTGGCTCACGCCTGTAA
TCCCAGCAGCACTTTGGGAGGCCGAGGTGGGCAGATCACGAGGTCAGAAG
ATCGAGACCATCCTGGCTAACACGGTGAAACCCCATCTCTACTAAAAATA
CAAAAAATTANCCGGGCGTGGTGGCGGGCGCCTGTAGTCCCAGCTACTCA
NGAGGCTGANGCAGGAGAATGGTGTGAACCCGGGAGGCAGAGGTTGCAGT
GAACCAAGATCGNNCCACTGCACTCCAGCCTGGGCAACAGAGCGAGACTC
CATCTTAAAAAA
SEQ ID NO: 485
AATCAGGGCCGCAGTGTGTTCTGCGCCTGCCCAGAGCTGACTCCTGATTT
AACCGCTGGCGTAACCGCGGGTTGCACGCATGCGTGCTGAAAAGCCTTTC
ACCCTCACGTGGTTTCTTTTTTAACCAGTCATCAAGCGAGGCTCGCGCGC
AGGCCCCGCGTTGGAAAATGGCGGGGAAGCTGAAACCTCTGAATGTGGAG
GCGCCAGAAGCTGCTGAGGAGGCTGAAGGTAGTGAGGGCAAGTGGGCTGC
ACTCCTTTCTCTCCAACCAGGGCAGAAAGGAGGGAGGATTCGTCCCATTA
CAATAATGAAATAATGATATTCTAATTTTTTTAAATAAAATGTTAAGCCT
TTTGTTATTGAA
SEQ ID NO: 486
GGAAANCATGAGGCTTCGGGAGCCGCTCCTGAGCGGCAGCGCCGCGATGC
CAGGCGCGTCCCTACAGCGGGCCTGCCGCCTGCTCGTGGCCGTCTGCGCT
CTGCACCTTGGCGTCACCCTCGTTTACTACCTGGCTGGCCGCGACCTGAG
CCGCCTGCCCCAACTGGTCGGAGTCTCCACACCGCTGCAGGGCGGCTCGA
ACAGTGCCGCCGCCATCGGGCAGTCCTCCGGGGAGCTCCGGACCGGAGGG
GCCCGGCCGCCGCCTCCTNTAGGCGCCTCCTCCCAGCCGCGCCCGGGTGG
CGACTCCAGCCCAGTCGTGGATTCTGGCCCTGGCCCCGCTAGCAACTTGA
CCTCGGTCCCAGTGCCCCACACCACCGCACTGTCGCTGCCCGCCTGCCCT
GAGGAGTCCCCGCTGCTTGGTAAGGACTCGGGTCGGCGCCAGTCGGAGGA
TTGGGACCCCCCCGGATTTCCCCGACAGGGTCCCCCANACATTCCCTCAG
GCTGGCTCTTCTACGACAGCCAGCCTCCCTCTTCTGGATCAGAGTTTTAA
ATCCCANACAGAGGCTTGGGACTGGATGGGAGAGAAGGTTTGCGAGGTGG
GTCCCTGGGGAGTCCTGTTGGAGGCGTGGGGCCGGGACCGCACAGGGAAG
TCCCGAGGCCCCTCTAGCCCCAAAACCANAGAAGGCCTTGGAGACTTCCC
TGCTGTGGCCCGAGGCTNAGGAAGTTTTGGAGTTTTGGGTCTGCTTANGG
CTTCNAGCAGCCTTGCACTGAGAACTTTGGTAGGGACCTCGAGTAATCCA
CTCCNTTTTNGGGACTGACGTGAGGCTCCCGGTGGGGAAAGANACTGACC
TNTC
SEQ ID NO: 487
CCGACCTGTCTCGCTCCGTGGCCTTAGCTGTGCTCGCGCTACTCTCTCTT
TCTGGCCTGGAGGCTATCCAGCGTACTCCAAAGATTCAGGTTTACTCACG
TCATCCAGCAGAGAATGGAAAGTCAAATTTCCTGAATTGCTATGTGTCTG
GGTTTCATCCATCCGACATTGAAGTTGACTTACTGAAGAATGGAGAGAGA
ATTGAAAAAGTGGAGCATTCAGACTTGTCTTTCAGCAAGGACTGGTCTTT
CTATCTCTTGTACTACACTGAATTCACCCCCACTGAAAAAGATGAGTATG
CCTGCCGTGTGAACCATGTGACTTTGTCACAGCCCAAGATAGTTAAGTGG
GATCGAGACATGTAAGCAGCATCATGGAGGTTTGAAGATGCCGCATTTGG
ATTGGATGAATTCCAAATTCTGCTTGCTTGCTTTTTAATATTGATATGCT
TATACACTTACACTTTATGCACAAAATGTAGGGTTATAATAATGTTAACA
TGGACATGATCTTCTTTATAATTCTACTTTGAGTGCTGTCTCCATGTTTG
ATGTATCTGAGCAGGTTGCTCCACAGGTAGCTCTAGGAGGGCTGGCAACT
TAGAGGTGGGGAGCAGAGAATTCTCTTATCCAACATCAACATCTTGGTCA
GATTTGAACTCTTCAATCTCTTGCACTCAAAGCTTGTTAAGATAGTTAAG
CGTGCATAAGTTAACTTCCAATTTACATACTCTGCTTAGAATTTGGGGGA
AAATTTAGAAATATAATTGACAGGATTATTGGAAATTTGTTATAATGAAT
GAAACATTTTTGTCATATAAGATTCATATTTACTTCTTATACA
SEQ ID NO: 488
TAAATAGGGAATCCTTTCCCCATTGCTTGTTTTTCTCAGGTTTGTCAAAG
ATCAGATAGTTGTAGATATGCGACGTTATTTCTGAGGGCTCTGTTCTGTT
CCATTGATCTATATCTCTGTCACATGCACACGTATGTTTGTTGTGGCACT
ATTCACAGTGGCAAAGACTTGGAACCAACCCAAATGTCCAACAATGATAG
ACCGGGTTAAGAAAATGCGGCACATATACACCATGGAATACTATGTAGCC
ATAAAAAATGATGAGTTCGTGTCCTTTGTAGGGACATGGATGAAATTGGA
AATCATCATTCTCAGTAAACTATCGCAGGAACAAAAAACCAAACACTGCA
TATTCTCACTCATAGGTGGGAATTGAACAGTGGGAACACATGGACACAGG
AAGGGGAACATCACACTCTGAGGACTGTTGTGGGGTGGGGGGAGGGAGGA
GGGATAGCATTGGGAGATATACCTAGTGCTGGATGACGAGTTAGTGGGTG
CAGCGCACCAGCATGTCACATGTATACATATGTAACTAACCTGCACATTG
TGCACATGTACCCTAAAACTTAAGGTAT
SEQ ID NO: 489
CCGCAACAAACACGGGAGTGCAGATATCGCTGCGATGGGCTGATTTCCTT
TATTTGGGTATATACCCAGCAGTGGGATTGCTGGATTGTATGGTAGCTCT
ATTAGTTTTTTGAGGAACCTCCAAACTGTTCTNCATAGTGGTTGTACTCA
TTTACATTCCCACTGTGAACCCTGAAAATTTGAGGCAGGTCTCAGTTAAA
TTAGAAAGTTGATTTTGCCAAGTTGGGGACACGCACTCGTGACACAGCCT
CAGGAGGAACTGATGACATGTGCCCAGGTGGTCAGAGCACAGCTTGGTTT
TATACATTTTAGGGAAACCTGAGCCATCAATCAACATACGTAAAATGGGC
CGGGCACAGCAGCTCAAGCTGTAATCCCAGCACTCTGGGAGGCCGAGGCG
GGTGGATCACTTGAGGTCAGGAGTTCGAGACCAGCCTGGCCAACATGGTG
AAACCCCGTCTCTATTAAAAATACAAAGCTTAGCTGGATGTGGTGGCGCA
TGCCTGTAGTCCCAGCTGCTCTAGGAGGCTGAGGCATGAGAATTGCTTGA
ACCTGGGAGGCAGAGGCTGCAGTGAGCCGAGATCGAGCCACTATACTCCA
GCCTGGTCAACAGAGTGAGACCCTGTCT
SEQ ID NO: 490
CCACAACTGTGTTCACTAGCAACCTCAAACAGACACCATGGTGCACCTGA
CTCCTGAGGAGAAGTCTGCCGTTACTGCCCTGTGGGGCAAGGTGAACGTG
GATGAAGTTGGTGGTGAGGCCCTGGGCAGGCTGCTGGTGGTCTACCCTTG
GACCCAGAGGTTCTTTGAGTCCTTTGGGGATCTGTCCACTCCTGATGCTG
TTATGGGCAACCCTAAGGTGAAGGCTCATGGCAAGAAAGTGCTCGGTGCC
TTTAGTGATGGCCTGGCTCACCTGGACAACCTCAAGGGCACCTTTGCCAC
ACTGAGTGAGCTGCACTGTGACAAGCTGCACGTGGATCCTGAGAACTTCA
GGCTCCTGGGCAACGTGCTGGTCTGTGTGCTGGCCCATCACTTTGGCAAA
GAATTCACCCCACCAGTGCAGGCTGCCTATCAGAAAGTGGTGGCTGGTGT
GGCTAATGCCCTGGCCCACAAGTATCACTAAGCTCGCTTTCTTGCTGTCC
AATTTCTATTAAAGGTTCCTTTGTTCCCTAAGTCCAACTACTAAACTGGG
GGATATTATGAAGGGCCTTGAGCATCTGGATTCTGCCTAATAAAAAACAT
TTATTTTCATTG
SEQ ID NO: 491
ATGGGCATCTCTCGGGACAACTGGCACAAGCGCCGCAAAACCGGGGGCAA
GAGAAAGCCCTACCACAAGAAGCGGAAGTATGAGTTGGGGCGCCCAGCTG
CCAACACCAAGATTGGCCCCCGCCGCATCCACACAGTCCGTGTGCGGGGA
GGTAACAAGAAATACCGTGCCCTGAGGTTGGACGTGGGGAATTTCTCCTG
GGGCTCANAGTGTTGTACTCGTAAAACAAGGATCATCGATGTTGTCTACA
ATGCATCTAATAACGAGCTGGTTCGTACCAAGACCCTGGTGAAGAATTGC
ATCGTGCTCATCGACAGCACACCGTACCGACAGTGGTACGAGTCCCACTA
TGCGCTGCCCCTGGGCCGCAAGAAGGGAGCCAAGCTGACTCCTGAGGAAG
AAGAGATTTTAAACAAAAAACGATCTAAAAAAATTCAGAAGAAATATGAT
GAAAGGAAAAAGAATGCCAAAATCAGCAGTCTCCTGGAGGAGCAGTTCCA
GCAGGGCAAGCTTCTTGCGTGCATCGCTTCAAGGCCGGGACAGTGTGGCC
GAGCAGATGGCTATGTGCTAGAGGGCAAAGAGTTGGAGTTCTATCTTAGG
AAAATCAAGGCCCGCAAAGGCAAATAAATCCTTGTTTTGTCTTCACCCAT
GTAATAAAGGTGTTTATTGTTTTTGTT
SEQ ID NO: 492
CTTNCACATACTGATTGATGTCTCATGTCTCTCTAAAATGTGTAAAACCA
AGCTGTGCCCCAACCACCTTGGGNACATGTGGNGAGGACCTCCTGAGGCT
GTGTCATGGGCACACCTTAACCCTGGGAAAATAAACTTTCTAAACTGACT
TGAGAGCTGTCTCAGATATTCTGAGCTTACAGTTATTGTGAAATCATTTT
AATTATAAATTAAGTGGAGATTTACTTAAAATCATGTGTAGAAGTAGCCT
GTGATATAGTCCTAGATACATACATTATCATCTTATGTATCTTCCCTCCC
TCTTCCAGGTTCTGATAAAAACAGATGAAATCTGAAAGACCATGACAGTA
GTATTTTGAAAATGACAGTATTTGAAATTAAAAAATTGTAAAAGTGTTCT
GTTCTATCACTGCCAAAGGATAAGTTACAAATTGGTTCTTGGAACGTAAT
ATGTACTATGTGCTTGCTATTTAATAATTTACCAGTCTTAGTCTTTTTTA
TTCAGACTAATTTTACCTTTTTTTAACCTATGACTCTTTAGTTATAGTAG
TACAAAAAAGTAGTTTTAGTTATAGTTTTAGTTGTAGTACAAAAAAGCAT
TTTCTGTAAGCTTAATTTCTTTCCCCTTCCCGCTTTCCCAGTCAGATGAC
TTTAGTGATTTGGAGTTGTGTGCTTTATAAGTGCATTCCTCAGAGGACTT
AATATTACTAAGATTTTAGCAACNCTGAAATATGTT
SEQ ID NO: 493
TGTNCCTGTAGTCCTGTGTGGGAGGATTGCCTGAGCCTAGGAGCTCAAAG
TTGCAGTGAGCCCAGATCGNGNCATTGCAGTCCAGCCTGGGTGACAGAGT
GAGACCCCATGTCAAAAAAAAAAAAACAAAAAACAGGGGCCTGCCTCANC
CAGCAGGTGAGGTCTGCCACTGAGAGCACTTCTAGCAGCAGGAACAGCCT
CCACCCCCACACTGCAATCAAGTTTTTTGGGTCAGCCTTAGGAGCTAANA
AAGGGCCTAGTTTGNCTAAATAGCAGGAGTTATATCCAGGGATCTTCAGG
CCCAGGAATGCTAATGAGTAGGCATTCCATGGGCCCTGGGAATGGCTTTG
TGTGCCANAAATGATGGCCACAAAGGCCTTGCTGCCTTTTTTCAAAATGG
CTGCATCCAGCTGAGTGCTCTCTGCCAAAGGGGANAANAAAATAAGTCTC
CAGTGCATTTAGATTGGTCTCTCATCATCTCTCTCCTTTTTGTTTTTATT
AGTCTCCTTAACCAAAACTGCCAAGAAAGGCTTGGAATTGAAACAAAACC
TGATANAANAGGTAAGAGGTTGTTCTTTT
SEQ ID NO: 494
TGTNTCAAAAAAAAAAAAAAGAACGGNAATGTACTGGAGATGTATTTGAT
AACCAAGGNTTTAGGTAAATTTTCACCAGTATTAGTTNTATTTGCAAACT
GAAAAATGTTGTAGGCTTAATATAAAATAACCACATTAGTGAACATTATA
TCTCTTAGAAGAAAGGCCATATTTTGCTCCTGCTTCTGTAAAAATATTAT
TTGTTTGAAGGGGAAATAATGGTAGTGTGACCTTTCACTTAATTCCTACT
CCCTTAATGTGAGAGAGACAAAATGAGCTGAAGAAGGAAAATTCTGGAGT
TACACTCCACAACCTTGAACATACTGACGGACATCTCTGTTTTGACAACG
ATTTCTCCATGCCACCCATGCTNTAATGCCTTGTGGATCACGGACAACCC
TCTTTGCACAAGCTACAGCATCAGCGATGTTATCTTGCAGCAAAGCACTG
CAGGATAAATGACAGGCATTAACTGCTCCTGGGGTTTTGCCATCATTACA
CCAGTAGCGGCTATTGATCTGAAATATCCCATAATCAGTGCTTCTGTCTC
CAGCATTGTAGTTTGTAGCTCGTGTGTTGTAACCACTCTCCCATTTGGCC
AAACACATCCAGTTTGCTAGGCTGATTCCCCTGTAGCCATCCATTCCCAA
TCTTTTCAGAGTTCTGGCCAACTCACACCTTTCAAAGACCTTGCCCTGGA
CCGTAACAGAAAGGAGGACAAGCCCCAGAACAATGAGAGCCTTCATGTTG
AC
SEQ ID NO: 495
TTGGTACCCGGGAAATTCTTTGCCGCGTCGACGGCCGGTGAGGCAGATCA
CCTGAGCCCAGGAGTTCAGGACCAGCCTGGGCAGCATACCGGGATTCCAT
CTNNACTAAAAACAGTAGGCTGGGTGTGGTGGCTCATGTCTGTAAGCTCA
GGACTTTGGAAGGCCAAGATGGGAGGATCACTTGAGCCTGGGAGTTTGAC
ACCAGCTTGAGCATCGTAGCCAGGCCCTGACTCTACAAAAAAGTGAAATA
ATTAGCCGAGTGTGGTGGTTCACACCTGTAATCCCAGCTGCTCAGGAGGC
TGAGGTAGGAGAATCATTTGAACCCGGGAGGTGGAGGTTGCAGTTAGCCG
AGATCACGCCATTGCACTCCGGCCTGGGCGATAAAGCGAGACTCTGTCTC
AAAAAAAAAAAAAA
SEQ ID NO: 496
ATTCGGGCCGAGATGTCTCGCTCCGTGGCCTTAGCTGTGCTCGCGCTACT
CTCTCTTTCTGGCCTGGAGGCTATCCAGCGTACTCCAAAGATTCAGGTTT
ACTCACGTCATCCAGCAGAGAATGGAAAGTCAAATTTCCTGAATTGCTAT
GTGTCTGGGTTTCATCCATCCGACATTGAAGTTGACTTACTGAAGAATGG
AGAGAGAATTGAAAAAGTGGAGCATTCAGACTTGTCTTTCAGCAAGGACT
GGTCTTTCTATCTCTTGTACTACACTGAATTCACCCCCACTGAAAAAGAT
GAGTATGCCTGCCGTGTGAACCATGTGACTTTGTCACAGCCCAAGATAGT
TAAGTGGGATCGAGACATGTAAGCAGCATCATGGAGGTTTGAAGATGCCG
CATTTGGATTGGATGAATTCCAAATTCTGCTTGCTTGCTTTTTAATATTG
ATATGCTTATACACTTACACTTTATGCACAAAATGTAGGGTTATAATAAT
GTTAACATGGACATGATCTTCTTTATAATTCTACTTTGAGTGCTGTCTCC
ATGTTTGATGTATCTGAGCAGGTTGCTCCACAGGTAGCTCTAGGAGGGCT
GGCACCTTAGAGGTGGGGAGCAGAGAATTCTCTTATCCAACATCAACATC
TTGGTCAGATTTGAACTCTT
SEQ ID NO: 497
GGATTTTTGGTCCGCACGCTCCTGCTCCTGACTCACCGCTGTTCGCTCTC
GCCGAGGAACAAGTCGGTCAGGAAGCCCGCGCGCAACAGCCATGGCTTTT
AAGGATACCGGAAAAACACCCGTGGAGCCGGAGGTGGCAATTCACCGAAT
TCGAATCACCCTAACAAGCCGCAACGTAAAATCCTTGGAAAAGGTGTGTG
CTGACTTGATAAGAGGCGCAAAAGAAAAGAATCTCAAAGTGAAAGGACCA
GTTCGAATGCCTACCAAGACTTTGAGAATCACTACAAGAAAAACTCCTTG
TGGTGAAGGTTCTAAGACGTGGGATCGTTTCCAGATGAGAATTCACAAGC
GACTCATTGACTTGCACAGTCCTTCTGAGATTGTTAAGCAGATTACTTCC
ATCAGTATTGAGCCAGGAGTTGAGGTGGAAGTCACCATTGCAGATGCTTA
AGTCAACTATTTTAATAAATTGATGACCAGTTGTTAAAA
nt: 362
SEQ ID NO: 498
CTTATTGAAAATTTTACTAATTTCTTACTTTTTAGGTTTTAGGAGAATAC
TTTTGGATAATTGACTAGCCTCACATTATATTGATAGAGGTTCTTGAAAA
CTTTAATGCCAATTCATGTATCTTATGACTAAAATAGATAATCCATTTAG
AAATTTAAGTCATTCTTGCGTGCTTGATATGTGTCAGCACTATCCAAGTT
GCTAGGGGATACAATGGTGAAGTGAAAATATCAGCTAGGTGCCGGTGGCT
CACACCTGTTATCCCAACAGTTTGGGAGGCCAGGGTGGGAGGATCACTCA
AGCACANGCGTTTCACACCAGCCTGGACAACATACAAGACCCCATCTTTA
CCAAAAGTTAAG
nt: 382
SEQ ID NO: 499
TTTTCTTAGAACTTTATTTTTTCTGGCCAGGCGCAGTGGCTCACACCTGT
AATCCCAGCACTTTGGGAGGCCAAGGCAGGTCGATCACCTGAGGTCAGGA
GCTCAAGACCAGCCTGGCCAACATGGTGAAACCCTGTCTCTACTAAAAAT
ACAAAAATTAGCTGGGCGTGGTGGCGCATGCCTGTAATCCCANCTACTCA
GGAGGCTGAGGCAGGAGAATTGTTTGAACCCGGGAGGCGGAGGTTGCANT
GAGCCGAGATTGCGCCACTGCACTCCAGCCTGGGCAACAGAGCGAAACTC
CATCTCAAAAAAAAAAAAAAAAAACAACCTTTATTTTTTCTGATTTTAAA
AGTAATAACTAGTTTGTAGAAACATTAAAAGT
nt 556
SEQ ID NO: 500
TCTTTCGGAAGCGCGCCTTGTGTTGGTACCCGGGAATTCGCGGCCGCGTC
GACGCGGTCGTAAGGGCTGAGGATTTTTGGTCCGCACGCTCCTGCTCCTG
ACTCACCGCTGTTCGCTCTCGCCGAGGAACAAGTCGGTCAGGAAGCCCGC
GCGCAACAGCCATGGCTTTTAAGGATACCGGAAAAACACCCGTGGAGCCG
GAGGTGGCAATTCACCGAATTCGAATCACCCTAACAAGCCGCAACGTAAA
ATCCTTGGAAAAGGTGTGTGCTGACTTGATAAGAGGCGCAAAAGAAAAGA
ATCTCAAAGTGAAAGGACCAGTTCGAATGCCTACCAAGACTTTGAGAATC
ACTACAAGAAAAACTCCTTGTGGTGAAGGTTCTAAGACGTGGGATCGTTT
CCAGATGAGAATTCACAAGCGACTCATTGACTTGCACAGTCCTTCTGAGA
TTGTTAAGCAGATTACTTCCATCAGTATTGAGCCAGGAGTTGAGGTGGAA
GTCACCATTGCAGATGCTTAAGTCAACTATTTTAATAAATTGATGACCAG
TTGTTT
nt: 464
SEQ ID NO: 501
GCGGCTGCTGTTGGTTGGGGGCCGTCCCGCTCCTAAGGCAGGAAGATGGT
GGCCGCAAAGAAGACGAAAAAGTCGCTGGAGTCGATCAACTCTAGGCTCC
AACTCGTTATGAAAAGTGGGAAGTACGTCCTGGGGTACAAGCAGACTCTG
AAGATGATCAGACAAGGCAAAGCGAAATTGGTCATTCTCGCTAACAACTG
CCCAGCTTTGAGGAAATCTGAAATAGAGTACTATGCTATGTTGGCTAAAA
CTGGTGTCCATCACTACAGTGGCAATAATATTGAACTGGGCACAGCAGCA
TGCGGAAAATACTACAGAGTGTGCACACTGGCTATCATTGATCCAGGTGA
CTCTGACATCATTAGAAGCATGCCAGAACAGACTGGTGAAAAGTAQAACC
TTTTCACCTACAAAATTTCACCTGCAAACCTTAAACCTGCAAAATTTTCC
TTTAATAAAATTTGCTTG

1. A set of oligonucleotide probes, wherein said set consists of not more than 1000 oligonucleotide probes and said set comprises at least 10 different oligonucleotide-probes, wherein each oligonucleotide probe is selected from:
an oligonucleotide having a sequence as set forth in SEQ ID NO: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 342, 343, 344, 347, 348, 350, 351, 352, 353, 354, 356, 357, 358, 359, 360, 361, 362, 363, 364, 365, 366, 367, 368, 369, 370, 371, 372, 373, 374, 397, 398, 399, 400, 401, 402, 403, 404, 405, 406, 407, 409, 415, 445, 447, 448, 451, 452, 454, 455, 456, 458, 459, 460, 461, 462, 463, 464, 465, 467, 468, 471, 472, 473, 474, 475, 480, 481, 482, 483, 484, 485, 486, 487, 488, 491, 492, 493, 494, 495, 496, 500 and 501,
with the proviso that at least one of said at least 10 oligonucleotide probes may be replaced,
wherein the one of said at least 10 oligonucleotides may be replaced with either
(i) an oligonucleotide fragment of the one of said at least 10 oligonucleotides which is to be replaced which is at least 20 nucleotides in length, which fragment is at least 20 nucleotides in length,
(ii) an oligonucleotide which has the complementary sequence to (a) the one of said at least 10 oligonucleotides which is to be replaced, or (b) a fragment of at least 20 nucleotides in length of the one of said at least 10 oligonucleotides which is to be replaced, or
(iii) an oligonucleotide having at least 80% identity to (a) the one of said at least 10 oligonucleotides which is to be replaced or (b) a fragment of at least 20 nucleotides in length of the one of said at least 10 oligonucleotides which is to be replaced, wherein said fragments do not bind to a polyA sequence.

2-3. (canceled)

4. A set of oligonucleotide probes as claimed in claim 1, wherein each oligonucleotide probe in said set binds to a different transcript.

5. A set as claimed in claim 1 consisting of from 10 to 500 oligonucleotide probes.

6. (canceled)

7. A set of oligonucleotide probes as claimed in claim 1, wherein each of said oligonucleotide probes is from 15 to 200 bases in length.

8. A set of oligonucleotide probes as claimed in claim 1, wherein the transcript to which said probe binds is derived from a gene which is constitutively moderately or highly expressed.

9. A set of oligonucleotide probes as claimed in claim 1, wherein said oligonucleotide probes are immobilized on one or more solid supports.

10. A set of oligonucleotide probes as claimed in claim 9, wherein said solid support is a sheet, filter, membrane, place or biochip.

11-12. (canceled)

13. A kit comprising a set of oligonucleotide probes as defined in claim 1 immobilized on one or more solid supports.

14. A kit as claimed in claim 13 wherein said oligonucleotide probes are immobilized on a single solid support and each unique probe is attached to different region of said solid support.

15. A kit as claimed in claim 13 further comprising standardizing materials.

16. A method for determining the gene expression pattern of a cell, comprising at least the steps of:
a) isolating mRNA from said cell, which may optionally be reverse transcribed to cDNA;
b) hybridizing the mRNA or cDNA of step (a) to a set of oligonucleotide probes as defined in claim 1; and
c) assessing the amount of mRNA or cDNA hybridizing to each of said oligonucleotide probes to produce said pattern.

17. A method of preparing a standard gene transcript pattern characteristic of Alzheimer's disease or a stage thereof in an organism comprising at least the steps of:
a) isolating mRNA from the cells of a sample of one or more organisms having Alzheimer's disease or a stage thereof, which may optionally be reverse transcribed to cDNA;
b) hybridizing the mRNA or cDNA of step (a) to a set of oligonucleotide probes as defined in claim 1 specific for Alzheimer's disease or a stage thereof in an organism and sample thereof corresponding to the organism and sample thereof under investigation; and
c) assessing the amount of mRNA or cDNA hybridizing to each of said oligonucleotide probes to produce a characteristic pattern reflecting the level of gene expression of genes to which said oligonucleotide probes bind, in the sample with Alzheimer's disease or a stage thereof.

18. A method of preparing a test gene transcript pattern comprising at least the steps of:
a) isolating mRNA from the cells of a sample of said test organism, which may optionally be reverse transcribed to cDNA;
b) hybridizing the mRNA or cDNA of step (a) to a set of oligonucleotide probes as defined in claim 1 specific for Alzheimer's disease or a stage thereof in an organism and sample thereof corresponding to the organism and sample thereof under investigation; and
c) assessing the amount of mRNA or cDNA hybridizing to each of said oligonucleotide probes to produce said pattern reflecting which reflects the level of gene expression of genes to which said oligonucleotide probes bind, in said test sample.

19. A method of diagnosing or identifying or monitoring Alzheimer's disease or a stage thereof in an organism, comprising the steps of:
a) isolating mRNA from the cells of a sample of said organism, which may optionally be reverse transcribed to cDNA;
b) hybridizing the mRNA or cDNA of step (a) to a set of oligonucleotide probes as defined in claim 1 specific for Alzheimer's disease or a stage thereof in an organism and sample thereof corresponding to the organism and sample thereof under investigation;
c) assessing the amount of mRNA or cDNA hybridizing to each of said oligonucleotide probes to produce a characteristic pattern reflecting the level of gene expression of genes to which said oligonucleotide probes bind in said sample; and
d) comparing said pattern to a standard diagnostic pattern prepared by
(i) isolating mRNA from the cells of a sample of one or more organisms having Alzheimer's disease or a stage thereof, which may optionally be reverse transcribed to cDNA;
(ii) hybridizing the RNA or cDNA of step i) to said set of oligonucleotide probes; and
(iii) assessing the amount of mRNA or cDNA hybridizing to each of said oligonucleotide probes to produce a characteristic pattern reflecting the level of gene expression of genes to which said oligonucleotide probes bind, in the sample with Alzheimer's disease or a stage thereof, wherein the sample is from an organism corresponding to the organism and sample under investigation, to thereby determine the degree of correlation indicative of the presence of Alzheimer's disease or a stage thereof in the organism under investigation.

20. A method as claimed in claim 18 wherein said mRNA or cDNA is amplified prior to step b).

21. A method as claimed in claim 18 wherein the oligonucleotide probes and/or the mRNA or cDNA are labelled.

22-27. (canceled)

28. A method as claimed in claim 18 wherein said pattern is expressed as an array of numbers relating to the expression level associated with each probe.

29. A method as claimed in claim 18 wherein said organism is a eukaryotic organism, preferably a mammal.

30. A method as claimed in claim 29 wherein said organism is a human.

31. A method as claimed in claim 18 wherein the data making up said pattern is mathematically projected onto a classification model.

32. (canceled)

33. A method as claimed in claim 18 wherein said sample is tissue, body fluid or body waste.

34. A method as claimed in claim 18 wherein said sample is peripheral blood.

35-37. (canceled)

38. A set of oligonucleotide probes as claimed in claim 1, where said set comprises each of the following 388 oligonucleotides having the sequences as set forth in SEQ ID NO: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 342, 343, 344, 347, 348, 350, 351, 352, 353, 354, 356, 357, 358, 359, 360, 361, 362, 363, 364, 365, 366, 367, 368, 369, 370, 371, 372, 373, 374, 397, 398, 399, 400, 401, 402, 403, 404, 405, 406, 407, 409, 415, 445, 447, 448, 451, 452, 454, 455, 456, 458, 459, 460, 461, 462, 463, 464, 465, 467, 468, 471, 472, 473, 474, 475, 480, 481, 482, 483, 484, 485, 486, 487, 488, 491, 492, 493, 494, 495, 496, 500 and 501, with the proviso that at least one of said 388 oligonucleotide probes may be replaced,
wherein the one of said 388 oligonucleotides may be replaced with either
(i) an oligonucleotide fragment of the one of said 388 oligonucleotides which is to be replaced which is at least 20 nucleotides in length, which fragment is at least 20 nucleotides in length,
(ii) an oligonucleotide which has the complementary sequence to (a) the one of said 388 oligonucleotides which is to be replaced, or (b) a fragment of at least 20 nucleotides in length of the one of said 388 oligonucleotides which is to be replaced, or
(iii) an oligonucleotide having at least 80% identity to (a) the one of said 388 oligonucleotides which is to be replaced or (b) a fragment of at least 20 nucleotides in length of the one of said 388 oligonucleotides which is to be replaced, wherein said fragments do not bind to a polyA sequence.


SEQUENCE LISTING (501 sequences)

SEQ ID NO: 1 - DNA, 405 bp, Homo sapiens
ggatcctgtggcccacagagctgccccagcagacgctccgccccacccggtgatggagccccggggggacaatcgtgcctggggaggagcagggtacagcccattcccccagccctggctgacctggcctagcagtttggccctgctggccttagcagggagacaggggagcaaagaacgccaagccggaggcccgaggccagccggcctctcgagagccagagcagcagttgaatgtaatgctggggacaggcatgctgccgccagtagggcggggacccggacagccaggtgactaccagtcctggggacacactcaccataaacacatccccaggcaggacagatcggggaaggggtgtgtaccaggctatgatttctcttgcattaaaatgtattattatt

SEQ ID NO: 2 - DNA, 550 bp, Homo sapiens
  misc_feature (61)..(61): n is a, c, g, or t
ggctttgacagagtgcaagacgatgacttgcaaaatgtcgcatctggaacgcaacataganaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccacggccacagtcatggtggccacggccacagccactaatcaggaggccaggccaccctgcctntacccaaccagggccccggggcctgttatgtcaaactgtcttggctgtggggctaggggctggggccaaataaagtctctttctcc

SEQ ID NO: 3 - DNA, 423 bp, Homo sapiens
acgaagacagacatctgtggaatgattcacatcctctcaagttaggaggatggaggcctgcttcattaagaagctgggggtagggtgggggtggggagaacacttaacaacatggggaccagtcaggggaatccccttatttctgttttgcatatgaggaaccctagagcagccaggtgaggctctctagtttaataaaaatcatggaaagactcttaatgcagactcttcttaagtgttaatagggattttttcagcttattttggttgcagtttccaatttttaaaaatgttgaggtaatctttcccaccttcccaaacctaattcttgtagatgcattagtgttgaaccaatgctttctcatgtctcaattctttgtatatgcattcttttcagatgtattaaacaaacaaaaacccttc

SEQ ID NO: 4 - DNA, 286 bp, Homo sapiens
  misc_feature (64)..(65): n is a, c, g, or t
ccggtaatagaatagaaaagggagagtgtcttcatgcaatgtggcatcctggattgggtctcgnnacaaaaacaggacattagtgggaaaattggaaatctgaaaaaagtctgaattttagttaatataccaatttcagtctcttggttttgacagatgtaccatggtgatgtaagatgttgaccttggggtaggctgggtgaagggtatacaggaactctttgtactatctctgcaacttctctgtaaatctagtatcattccaaaataaaagtttatttaattt

SEQ ID NO: 5 - DNA, 545 bp, Homo sapiens
gtggaagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcactgatcaagactgg

SEQ ID NO: 6 - DNA, 591 bp, Homo sapiens
  misc_feature (85)..(85): n is a, c, g, or t
cagcgcaggggcttctgctgagggggcaggcggagcttgaggaaaccgcagataagtttttttctctttgaaagatagagattgntacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcanggagaattgcgcatttaaagcctagttacgcatttactaaacgcagacgaaaatgggaagattaattgggagtggtaggatgaaacaattttggagaagatagaag

SEQ ID NO: 7 - DNA, 297 bp, Homo sapiens
ctcaaaggagaaaaaaaaccttgtaaaaaaagcaaaaatgacaacagaaaaacaatcttattccgagcattccagtaacttttttgtgtatgtacttagctgtactataagtagttggtttgtatgagatggttaaaaaggccaaagataaaaggtttctttttttttccttttttgtctatgaagttgctgtttattttttttggcctgtttgatgtatgtgtgaaacaatgttgtccaacaataaacaggaattttattttgctgagttgttctaaaaaaaaaaaaaaaaaaaaa

SEQ ID NO: 8 - DNA, 282 bp, Homo sapiens
agtagagacggggtttcactgtgttagccaggatggtctcgatctcctgacctcgtgatccggccacctcggcctcccgaaagtgctgggattacaggcgtgagccacggcgcccagccccagcctgtcacttaaactgataaacgacagattaacagtagaaaaattttattttgcatacataatgaggcttcacaaaagagaagtgaaaacccaagtaggagtttagggctgggggcttatataccatttaacaaggggtgataaattgtaagagaatag

SEQ ID NO: 9 - DNA, 619 bp, Homo sapiens
tccttggtttcgatttgtggcaacaatccagtctttttgtttttttcagggataccatatgtaacaggtgccattgttactgtaacttttcacacatgccttcagtttgatgtcaaagtcatcatttagtgtaaacagcaagttatctgttaggctgcacatcatgaactttacttttagaaagtcttatcttttatgccacagaaatagcatttggctattagtcatggatggcaaagaaattaattttgagttgtttggataaaaatgtttcagttgactgtagtgtgtattgagagacactgccagtaaacaaactctcttggtaggtggaaatcccctagaagttacagaaaattgggaggaggtgaacttaattaaataacttgaattgtttagacatattcagagcttcttatgaccttgaagaaatcacccaacttcaaaagacctcggtttcttcatttgtaaaattagggagtttgactagatgtgtaaatctagttgttagttaacttctaagatgtaaaaaccctcttgtttaacaaaaacctacaagatcaagttgcttatctgaaatctttatgaatcaacactagtcactaagtctagctcgacc

SEQ ID NO: 10 - DNA, 536 bp, Homo sapiens
  misc_feature (513)..(513): n is a, c, g, or t
cttttcctcccgctgtcccccacggaggggactgctctcccccgctgcatcctttctgtgaggtaccttacccacctcagcacctgagagggtgaaatagaattctaacctcgacattcgggaagtgtttttgagaagtctcggtcggtaagggaagtcttccaagtccgtgcagcactaacgtattggcacctgcctcctcttcggccaccccccagatgaggcagctgtgactgtgtcaagggaagccacgactctgaccatagtcttctctcagcttccactgccgtctccacaggaaacccagaagttctgtgaacaagtccatgctgccatcaaggcatttattgcagtgtactatttgcttccaaaggatcaggccctgagaacaatgaccttatttcctacaacagtgtctgggttgcgtgccagcagatgcctcagataccaagagataacaaagctgcagctcttttgatgctgaccaagaatgtggattttgtgaaggatgcncatgaanaaatggacnagctgtg

SEQ ID NO: 11 - DNA, 373 bp, Homo sapiens
  misc_feature (27)..(27): n is a, c, g, or t
aagtgggtcttgccatccctgaactgnaatcatccctaacatattcatacctgttttcattttaaaagttgggtcagtttttttattagtacatgtatttctatcctactgatttatttgctatatcatctaatttagtttgaatattccataatttacttaattagtcctgtatggagacctagctcttctcagtgtctactattataaacaatgctacagtgaatattggtgnataaatccatacncaccacgtacatatcttaagttctggaagagatattgctaaaccagaagataacctgcatttaaaatttgactgctagggncagggncacatttaattaaattagaacaangaatgcataatgnc

SEQ ID NO: 12 - DNA, 796 bp, Homo sapiens
  misc_feature (601)..(601): n is a, c, g, or t
ccggaatcgcggccgcgtcgacgaaaatatgtgccctggccaactccacaggactagttctaggcaatctgaaggaaaccagaaaatgtgaatttctcttccctcaaaaagctatactgaagtagtatttaatattcaagtacttgtaaatttgcagaacagtactttttaatttgacccatgaattctatttaaatttgtcacttaatatttagccaagaagcaaaccatctaaaaagatttctggtttatttctccaactcctaataaatagggtcacatattttttaacttttttctaatttgaaaagtaatacaggcatatggtattttaaaaatgaaacaacacaaagggatatgttttgaaaagtggtcttgccatccctgaactgtaatcatccctaacatattcatacctgttttcattttaaaagttgggtcagtttttttattagtacatgtatttctatcctactgatttatttgctatatcatctaatttagtttgaatattccataatttacttaattagtcctgtatggagacctagctcttctcagtgtctactattataaacaatgctacagtgaatattggtgnataaatcctacacaccacgtaacatatcttaagttcctggaagagatattgctaaaccagaagataacctgcatttaaaatttgactgctagggtcagggtcacatttaaattaaattagaacaaggaatgcataatgtcttcgatagcaatctattcaaggtgcaccgtggtcacaaaggaaagcaaaactgtc

SEQ ID NO: 13 - DNA, 564 bp, Homo sapiens
  misc_feature (6)..(6): n is a, c, g, or t
cctggncagaggcctctatcctgtantgataattgccatcaaaattgtcaaaaangatttaatttctatgggnaatagtccttttcttagcttctgccnntcacttgcttattttttgtgtgggaatggggttggataaaccaatgaactttattataaacaaatcccacctatatctancaaatttatattttcggtgaaatacagatatttgcctttctggagtantatagaagctgtcaatatgtatctactgtacagtactaaatagtattcatttatgaaatgagtagtgtttgggtggctggggttaaggaaaaatgagacttggaattgtagcttttatccaagttttgagtataaatagggttttgttttgttttttttaacctaaaaactgaaatgccatatagaaaaacagcattgtttttacagtttgtagtaagtaactttttaaagattttatcaaaaagaattttgtctatngtgagtaaaagaagttctaataatggcctaatcactgcatttttaaaaaacaaagttcaacacaaatgacatttgttt

SEQ ID NO: 14 - DNA, 230 bp, Homo sapiens
cctctcctccatctaaaggcaacattccttacccattagtctcagaaattgtcttaagcaacagccccaaatgctggctgcccccggccaagcattggggccgccatcctgcctggcactggctgatgggcacctctgttggttccatcagccagagctctgccaaaggccccgcagtccctctcccaggaggaccctagaggcaattaaatgatgtcctgttccattgg

SEQ ID NO: 15 - DNA, 554 bp, Homo sapiens
  misc_feature (149)..(149): n is a, c, g, or t
cccggaatcgcggcccgcgtcgacaacaaacctgcatgttctgcacatgtatccaggaacttaaaaaaaaaaaaagatagtttgtgtgtcttaattgaataatagtagatttatagattaaagatctatgggtttttaatatggattanaaatctgtgggtttttgatatggattanaaatctgtgggtttttaatatggattggaaatctgtgggtttttaatatggattaaaaaacatctgtgggtttttaatatggattaaacatctgtgggtttttaatatggattaaacatctgggtttttaatatggattaaacatctgtgggtttttaatatgggttaaaaatcaaaagaaaatgaactatttgctccagtgcaggaaaatacaggcaatactggatacaattagatggtcaggagcgataacccggttgccattgtttgaagaagagaataaggngctagcattcctatccgtagataatttgacagctaggaaatagggggagtcttctatgtagttagtgaaggctaaatgaactattatatgc

SEQ ID NO: 16 - DNA, 610 bp, Homo sapiens
cttttcctcccgctgtcccccacggaggggactgctctcccccgctgcatcctttctgtgaggtaccttacccacctcagcacctgagagggtgaaatagaattctaacctcgacattcgggaagtgtttttgagaagtctcggtcggtaagggaagtcttccaagtccgtgcagcactaacgtattggcacctgcctcctcttcggccaccccccagatgaggcagctgtgactgtgtcaagggaagccacgactctgaccatagtcttctctcagcttccactgccgtctccacaggaaacccagaagttctgtgaacaagtccatgctgccatcaaggcatttattgcagtgtactatttgcttccaaaggatcaggccctgagaacaatgaccttatttcctacaacagtgtctgggttgcgtgccagcagatgcctcagataccaagagataacaaagctgcagctcttttgatgctgaccaagaatgtggattttgtgaaggatgcacatgaagaaatggagcaggctgtggaagaatgtgacccttactctggcctcttgaatgatactgaggagaacaactctgacaaccacaatcatgagg

SEQ ID NO: 17 - DNA, 359 bp, Homo sapiens
tggtacagatacaaactggactctcaggacaaaacgacaccagccaaaccagcagcccctcagcatccagcagcatgagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtacgggccaagcactgcccagctgggggtcaataaagttacccttgtacttg

SEQ ID NO: 18 - DNA, 154 bp, Homo sapiens
cgccacttatccagtgaaccactatcacgaaaaaaactctacctctctatactaatctccctacaaatctccttaattataacattcacagccacagaactaatcatattaaaaaaaaaaaaaaaaaaaaaaaaaaaaaaaaaaaaaaaaaaaa

SEQ ID NO: 19 - DNA, 661 bp, Homo sapiens
cagaacagtactttttaatttgacccatgaattctatttaaatttgtcacttaatatttagccaagaagcaaaccatctaaaaagatttctggtttatttctccaactcctaataaatagggtcacatattttttaacttttttctaatttgaaaagtaatacaggcatatggtattttaaaaatgaaacaacacaaagggatatgttttgaaaagtggttcttgccatccctgaactgtaatcatccctaacatattcatacctgttttcattttaaaagttgggtcagtttttttattagtacatgtatttctatcctactgatttatttgctatatcatctaatttagtttgaatattccataatttacttaattagtcctgtatggagacctagctcttctcagtgtctactattataaacaatgctacagtgaatattggtgtataaatccatacacaccacgtaacatatcttaagttcctggaagagatattgctaaaccagaagataacctgcatttaaaattttgactgctagggtcagggtcacatttaaattaaattagaacaaggaatgcataatgtcttcgatagcaatctattccaggtgcaccgtggtcacaaaggaaagcaaaactgtcaataactttcttctca

SEQ ID NO: 20 - DNA, 770 bp, Homo sapiens
  misc_feature (557)..(557): n is a, c, g, or t
tagcatttggccttttaaaacatttgtttattttttttctgagaatggctaacacactttattgaggttcgaaattaataaagaaaataaaagaaatgtatcttcattcattctgtatgttagtgttttaattacccttagaatatatggataaaaaatactattctttgtcttggagaaggtaagagtctagttagatgaataagggttatctatgtagaacaactagagaatgagaagagagcttatgagattgagtactacgttatgcagtagagtagcacgtcatctgctactgagtatggtgtgataacattgtgtaacaggaaagtatgatcaatatctacttaaaattaaggacaatattagcactacattgctttattttaaagtaaaaattagagaactaaacacaagcattgtaagtacaataaaagctgatctttctagttaagcagaataatacatgttcaagcatctgctaaatcattaaatataagaatataggggttttctataatcttattttctttggaagagtacctcattttcaagangagaagtttctaattgccacttctttaaaaataaaacagggttttaatgttcccagcacaaaaattaatatctcttcaaaaagtctcttgtgattaagtttgaatcccttgtcatactgcttctaatattgacactgacctccttaggtatttttcaggggttataatcttttcttaaggtatcttttttcaagaattggataccttgggctt

SEQ ID NO: 21 - DNA, 654 bp, Homo sapiens
cgcgtcgacttttaaagtcatctctataggaaggtgctgggcagggatcccagagaaagaaagggtccaagactccattaactgccctggatgaagggcactgctacagcagctagtaccagagactctcctatctcacggttgaggcagacccaggatagaatagagaataaaaggaatgcttataggaaacaattttgtatggaatgctagatggccaagcctcagcctttggtccagtgcaacccttgcctcgcttgtcaacagtgaaaaattagtttggttagaagaaccatctggaaacacaccagcttctgctaccttcatgctcattgttaaaaaaagattaaccagtgtgaacattctgatctgttaattccagggactgttttctttccaatggactgtttgttggtagaataacccccaaaagctcaaagctaaaatgcatcatcagtcctagtcggcagttccttaagaatggactggcggcgtggttgagctgatatggaaaagctgcaccttcctgcagaagatcaactgacctgctatcccaccccaaattcaacctgaggtatatttcagtgaagcaggtagctgtgcttctcaaagcagagaagcagttttaagaaccaaaaaggtagaggaaatcta

SEQ ID NO: 22 - DNA, 676 bp, Homo sapiens
  misc_feature (199)..(199): n is a, c, g, or t
gtttgttacaggcagaattggatagatacagccctacaaatgtatatgccctcccctgaaaaaaattggatgaaaatctgcacagcaaagtgaaacacacagataataggaacaaaatgtagttcccatgtgccaaacaaaataaatgaaatctctgcatgtttgcagcatatctgccttttgggaatgtaatcaaggnataatctttggctagtgttatgtgcctgtatttttttaaaatggtacaccagaaaaggactggcagtctacttctaccatagttaaacttcaccctctttaatttcacaacatattctttggaagcaggaagaaatgctcataaagaggatcagaccttctttcccgtgaaaccagtatttggcgccatatataagcctggttaaattggtcatctaaagctgtcaaataagacattctgtgaaaggtaaacatcgaaactggttataagtaaaaccatcaagccaacaacagggtcttgagataacctttgaagcttattgtctggcctgcaccagaagatgtctgcattactcattgctaaaaatgtgtacacagaactgcactaggattaattggttcaagaagaaatttaaacttacgtttgggtttccatacagcactctattgaatacatgcatctgaatttaagttgcaa

SEQ ID NO: 23 - DNA, 609 bp, Homo sapiens
gaccagtaatggcttttaagagtccattttgtcattgtctccctagttaattacaggtgggggatcttttgcctctattctcttcatattgaaatgaatcatactcatgttttgtggaactccttaaagttgtagctgtcatgatcagattttttttatatttcctcagcttaactctgctacttgatttacagtgacccataacctactcatccttggtttatagtgacacataatcttatctctttatagaaccttaaattttatcattattttcgcttagaatacagcatttctttgcttctgttgctggtttgacttaagaaataaggcagtaactctgatcaatcaattatccataaggaagggcttttcatgggttctattaatttgttagtaccctaagtatatctgaaaaatatgtctattgagagaagattttggcattccagatggtatagtctatatatatttaaagttttgaatttgcttatatatactcagctttctttttctagcatttttgcatttacctgttaattgaagtataccccccacatataaaagttcctcttaaagacactggactctttctggggggctaaaata

SEQ ID NO: 24 - DNA, 554 bp, Homo sapiens; identical to SEQ ID NO: 15
  misc_feature (149)..(149): n is a, c, g, or t

SEQ ID NO: 25 - DNA, 674 bp, Homo sapiens
  misc_feature (388)..(388): n is a, c, g, or t
cggctaccgacagaaggactatttcatcgccacccaggggccactggcacacacggttgaggacttctggaggatgatctgggaggggaagtcccacactatcgtgatgctgacggaggtgcaggagagagagcaggataaatgctaccagtattggccaaccgagggctcagttactcatggagaaataacgattgagataaagaatgataccctttcagaagccatcagtatacgagactttctggtcactctcaatcagccccaggcccgccaggaggagcaggtccgagtagtgcgccagtttcacttccacggctggcctgagatcgggattcccgccgagggcaaaggcatgattgacctcatcgcagccgtgcagaagcancagcagcagacaggcaaccaccccatcaccgtgcactgcagtgccggagctgggcgaacaggtacattcatagccctcagcaacattttggagcgagtaaaagccgagggacttttanatgtatttcaagctgtgaagagtttacgacttcagagaccacatatggtgcaaccctggaacagtatgaaatgtgctacaaagtggtacaagatttattgatatatttctgattatgctaatttcaatgaagatcctgccttaaatattttttaatttaatggcanat

SEQ ID NO: 26 - DNA, 609 bp, Homo sapiens
  misc_feature (524)..(524): n is a, c, g, or t
caagactccatctcaaaaaaaaaaaaaaatctacagtgctgagtatataaaattattaacacatttcacaacaatatgtgtttgtggagttaaatattttttgtctttaaaacaggtaattttagtgcatacttaatttgatgattaaatatggtagaattaagcattttaaatgttaatgtttgttacattgttcaagaaataagtagaaatatattcctttgttttttatttaaatttttgttcctctgtaaactaaaagaacacgaagtaattggtcacaattactggtgtttaactgccaaatatgggtaaataagggaaaattttgtttaatatttagtccttctgagatggcttgaatatttgaattttgttgtacgtctatactgggtagtcacaagtcttataaacactttagaggaaagatggatttcagtctgtatttttaaacatcatttattttaaatctggtgctgaaaaataagaaaaaaattaaactgcattctgctgttcttctttanaagcattcctgcgtaaatactgctgtaatactgtcatgcaaagtgtatcctttcttgtcgtatcctttttggggcagtggttttt

SEQ ID NO: 27 - DNA, 383 bp, Homo sapiens
gcgggaatcgcggcccgcgtcgacctcaaaggagaaaaaaaaccttgtaaaaaaagcaaaaatgacaacagaaaaacaatcttattccgagcattccagtaacttttttgtgtatgtacttagctgtactataagtagttggtttgtatgagatggttaaaaaggccaaagataaaaggtttctttttttttccttttttgtctatgaagttgctgtttattttttttggcctgtttgatgtatgtgtgaaacaatgttgtccaacaataaacaggaattttattttgctgagttgttctaaaaaaaaaaaaaaaaaaaaaaaaaaaaaaaaaaaaaaaaaaaaaaaaaattttaaaatttttaaaataaaacccttggttat

SEQ ID NO: 28 - DNA, 729 bp, Homo sapiens
  misc_feature (213)..(213): n is a, c, g, or t
gccgcgtcgacctgcatgagccacagtttcttgactggaggccatcaaccctcttggttgaggccttgttctgagccctgacatgtgcttgggcactggtgggcctgggcttctgaggtggcctcctgccctgatcagggaccctccccgctttcctgggcctctcagttgaacaaagcagcaaaacaaaggcagttttatatgaaagattanaagcctggaataatcaggctttttaaatgatgtaattcccactgtaatagcatagggattttggaagcagctgctggtggcttgggacatcantggggccaagggttctctgtccctggttcaactgtgatttggctttcccgtgtctttcctggtgatgccttgtttggggttctgtgggtttgggtgggaagagggccatctgcctgaatgtaacctgctagctctccgaagccctgcgggcctggcttgtgtgagcgtgtggacagtggtggccgcgctgtgcctgctcgtgttgcctacatgtccctggcttgttgaggcgctgcttcaacctgcacccctccttgtctcatagatgctccttttgaccttttcaaaattaatatggatgggaaagctcctatgccttttggcttcctggtagaaggcgggatgcccaagggtctgcctgggtgtggattggatgcttggggtgtgggggttggaaactgtcttgtggcccacttgggcccc

SEQ ID NO: 29 - DNA, 552 bp, Homo sapiens
cccgcgtcgacttttaaagtcatctctataggaaggtgctgggcagggatcccagagaaagaaagggtccaagactccattaactgccctggatgaagggcactgctacagcagctagtaccagagactctcctatctcacggttgaggcagacccaggatagaatagagaataaaaggaatgcttataggaaacaattttgtatggaatgctagatggccaagcctcagcctttggtccagtgcaacccttgcctcgcttgtcaacagtgaaaaattagtttggttagaagaaccatctggaaacacaccagcttctgctaccttcatgctcattgttaaaaaaagattaaccagtgtgaacattctgatctgttaattccagggactgttttctttccaatggactgtttgttggtagaataacccccaaaagctcaaagctaaaatgcatcatcagtcctagtcggcagttccttaagaatggactggcggcgtgggtgagctgatttggaaaactgcccttctgcaaaaaacactggcctgctttcca

SEQ ID NO: 30 - DNA, 606 bp, Homo sapiens
caagactccatctcaaaaaaaaaaaaaaatctacagtgctgagtatataaaattattaacacatttcacaacaatatgtgtttgtggagttaaatattttttgtctttaaaacaggtaattttagtgcatacttaatttgatgattaaatatggtagaattaagcattttaaatgttaatgtttgttacattgttcaagaaataagtagaaatatattcctttgttttttatttaaatttttgttcctctgtaaactaaaagaacacgaagtaattggtcacaattactggtgtttaactgccaaatatgggtaaataagggaaaattttgtttaatatttagtccttctgagatggcttgaatatttgaattttgttgtacgtctatactgggtagtcacaagtcttataaacactttagaggaaagatggatttcagtctgtatttttaaacatcatttattttaaatctggtgctgaaaaataagaaaaaaattaaactgcattctgctgttcttctttagaagcattcctgcgtaaatactgctgtaatactgtcatgcaaagtgtatcctttcttgtcgtatcctttttggggcagtggtt

SEQ ID NO: 31 - DNA, 734 bp, Homo sapiens
  misc_feature (448)..(448): n is a, c, g, or t
ctggactgcatgaccagatctgatgggtgagactcaggtggcatggaagagccgaaagaggataccatatgtgggtgccgggggggataggtgagaagtactagaaggcggaatggaaggacacttctgctcagctctgtgacacgggcagggaccctgcagggctcaggtcctttaacacagcagcttcattctaacaccagcagcgttggaacacacgtacaagtatgcagactaagctcttgcttggctgatacggctttttgggtttttagagaacatgcatatatgttctcattcatggtacatgaactcagaagccttactgcctatttttgttaatacttctgggcaaacattaccacttacaactcacaccagttagaaatcatttgtaaaatgttatttaataaagccaaagaactaaatcatatttattttccaaggntttctaagatctctgaaactaatgaggttttttaaatccccattaagtactcatcactgctagtaaaagcagttgtctttacctttaattccagtgagtccccttaaatttattttttattatctttggctacattgccttagacaaaatgtggtcaccctaatttaanggataaaattcacatcctcacagatttcttattaagagggtctaanccttgaataatcancagtggaaatggaagtcttctttactggntttnatcctttcccttttttatcccatg

SEQ ID NO: 32 - DNA, 517 bp, Homo sapiens
  misc_feature (53)..(53): n is a, c, g, or t
tttttttttaaataaagctgtcggcactcaagggtaatttcatatcagtgtgntctacaagctgggggaaaatgagttctaattgtcanagctaccaaatccttcacctttagcataaaggtttaaagatatcacaaagatgccaagtgattaataatgttttaaaccacccctttttctgtctgaaaaaacaactaaaacaatattacaacagtatagttacagaagggttctattttcatatgttttatgcacactgtgcctcaaaggtactatttaaatatatatacttttgagggggtggctaatgcagaaacacccaagacctaaggaagatacaaccccatttctaggtgtgaggtctaaatgcttcacacacccacttgtgaccttttttcatgaagaatcataacactgtgcagtgagaaacagtggcaaagcaatactgaaagcattttaaattatttactaggttaaaagggtgaactgatactttaaatacatcaaatttcatcat

SEQ ID NO: 33 - DNA, 536 bp, Homo sapiens
gcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggta

SEQ ID NO: 34 - DNA, 622 bp, Homo sapiens
  misc_feature (5)..(5): n is a, c, g, or t
ctgtnatngaatctgcttgtnactnaaatgctaaactcaattctgtaattcaataggtgcacctntctgagaaacatannagacaatgaggaaaaggattcancattccgtggaatttgtaccatgatcagtgtgaatcccantggcgtaatccaagtaagatgttcacaaagatttgtttttaatgtctaattaataaaattttaaaggaagaaacattctaatactttaattataaaaagttaactattttcaaaggtatcaaaatacagttaaacctttaaaatgtatatttcttaatatcttgaaattgtaatgccttttttttttcctaaattttttttgtcatgaaatgagatagtaacagcagattgggacaacaaggttatattcttgtcttgaatcaggccatggcttctttcatccaaatttcagacctcatttatttactttgtccctgcctcccatccctggatatcangtttgtggatatctacagttaatagagtgaccaaatagtaggaatactgtctctctattctgaataaaatactttgaatcagatttagaaataatgaataaaatacaaatcaccattgaaattgctctaattttgagagct

SEQ ID NO: 35 - DNA, 628 bp, Homo sapiens
  misc_feature (6)..(6): n is a, c, g, or t
atcacntgaggcaagagtttgagccagcctagctaacatggtgaaaccccatctctacaaaaatataaaaattagcctgggtggtgatgggcacctgtaaccccagctactcgggaggctgaggtaggagaatcacttgaacccgggagatggaggttgcagtgagccaagatcgtgccactgcactccagcctgtgtgacagaacaagactctgtctcaaaaaaaaataataataataataataataaaaaggaataacatagctaggaataaatttaatcaaagaggtgaaagacttatacacttaaaactacaaaaaaaaaatcactgaaggaattatagacccaaataaaaataaataaaaagacattctgtgttttagggaaagaagacttaatattgttaagatgtcaatactacccaaagtgatctacagattcaacataatccctatcaaaattccaacagcctactttgtagaaatggaaaagccaattttcaaattcagatggaattgcgaggggttctgaataacaaaaacaatcttggggaaaaaaaacaaaaaacaaagtcaaagaactcacacttctctatttataaatttactacaaagttatagtaatcaaa

SEQ ID NO: 36 - DNA, 527 bp, Homo sapiens
  misc_feature (525)..(525): n is a, c, g, or t
tgaacatccagccatgtcatttcttccattcctgccctggagtaaagtagatttactgagctgatgacttgtgtgcatttgtacattgcaaccttagcttacctcttgaagcatgtagagcattcatcacccaccattcattcactgcctactcccaccacagctgtttcgtggtctgtctgctccctgtgccacccccaccccatcaggtgggccttttgcaagtgatgaagtcacctgtgggggaagagctttcctttcctctcctcaactcagaaggcctcttcctcttgctcaagagggtgctgctgctttctgcctccttccccggccggcctccatcccagttcaccttttcagaaatggcccctcagtcaactcttcccttttctcctggctttttatttctcccagtctcttaagagtatccttagctttaaaaacaataacacagaggatgggtgcagtggctcatgcctgtaatcccagcactttggagcctggggcgggcggatcacttgaggnca

SEQ ID NO: 37 - DNA, 403 bp, Homo sapiens
gtcccggaatcgcggccgcgtcgaccttttctatgcctgctatataaacagtaccttgcaagatgtcctgtctgatatccacaaaggggtattgtcaaccccaagttcagacagctttgtattcttctgtccctggatacatgaattactgccatctttacacagcgccctaaaataccaacgcgaagttacctgctcagcttgaagctgcgctgtaccctggaaccagcacttctgctgaatgactcaggatgaagcctcgacttctccttcccatcccatgcccagaccccagtggctcctttcccaatctgatccagtgactttaagtccagctgttgcaacctgggcatgaggaggagtgcaagatggctttgtcctacctggaaagaggctttctgga

SEQ ID NO: 38 - DNA, 111 bp, Homo sapiens
caccatttacacacagtgggtccttgaatagcatcgttttattcaatgtcattttgttataacattgagaaaaaaattgattcccggctggggccactgtctgtgcaccgt

SEQ ID NO: 39 - DNA, 329 bp, Homo sapiens
gaaagatctaaaatcgacaccctaacatcacaattaaaagaactagagaagcaagagcaaattcaaaagctagcagaaggcaagaaataactaagatcagagcagagctgaaagagatagagacacaaaaaaccattcaaaaaaaaacaatgaatccaggagttttttttttaaaaagatcaacagaattgacagactgctagcaagactaataaagaagagagaagcatcaaatagactcaataaaaaatgataaaggggatatcaccaccaatcccacagaaatacaaactaccatcagagaacactataaacacctctatgcaaat

SEQ ID NO: 40 - DNA, 341 bp, Homo sapiens
gaaagatctaaaatcgacaccctaacatcacaattaaaagaactagagaagcaagagcaaattcaaaagctagcagaaggcaagaaataactaagatcagagcagagctgaaagagatagagacacaaaaaaccattcaaaaaaaaacaatgaatccaggagttttttttttaaaaagatcaacagaattgacagactgctagcaagactaataaagaagagagaagcatcaaatagactcaataaaaaatgataaaggggatatcaccaccaatcccacagaaatacaaactaccatcagagaacactataaacacctctatgcaaataaactagaaaat

SEQ ID NO: 41 - DNA, 185 bp, Homo sapiens
gaaagatctaaaatcgacaccctaacatcacaattaaaagaactagagaagcaagagcaaattcaaaagctagcagaaggcaagaaataactaagatcagagcagagctgaaagagatagagacacaaaaaaccattcaaaaaaaaacaatgaatccaggagttttttttttaaaaagatcaaca

SEQ ID NO: 42 - DNA, 553 bp, Homo sapiens
  misc_feature (119)..(119): n is a, c, g, or t
gcccggaatcgcggccgcgtcgacgtaagctcggctgaatccacggttcaagaacaggaaagaaggccaaggcatagggagtggggcagttgggtgaatattagtacctttccctcagntncattaattacccctgcctactctgcacaaaaggatntaacaacagtttcctttttaatggccaggtacagctgcttatatggangggcatttntnaatgatatccttnatcactgtcttaatcatcacatncttaaaacaatcactttattgtgttaaggaagataaaaatggctgggttcaatttccgttctggaagaaatcgantnaaaaggtaaccatttaataatgcanagggcantttcactgcagaccctaatactggaaatttttaaaaacaaatgaaaaacttctactttttcttctaagcttacttaaccacccaaattttccagccacatatcttcctagtctacaactgcctttaactttaagagatgctcaaaaaaatgtaaattctcaaatacattcttattacaattactgctaacct

SEQ ID NO: 43 - DNA, 510 bp, Homo sapiens
ccagtgtgctgggattacaggcatgagccctgcacccagcctcttaaactgatcatatgatattggttctcaaccaagggtgactttgcccccagaggatacttggcaatgtctggagatactcagttgtcatgacttggacaggtgctactgtcacccagtgggtagaggtcagggatggtgctaaacataggacagctgtcaagagaaaagaatgtacccagccccaaatgtcagtagggctgaggttgagaaacccagctgtagctgacgtgtgaaggacagactggcctggaagtgtgttttctgcccctttccacccctgcatattagttaaggccaaaggaaaaaaggaatgcaggaaatgcccgttaaaaatcttcaaaacaatataaaatgatcaattccactaaaaccctttacacatttaagtataaaggtattggtaggaaaatttgttattcactgcttttctcagtgtcatgaaataattatttctgctgtcagttt

SEQ ID NO: 44 - DNA, 335 bp, Homo sapiens
aaaaaaaaaatcactgaaggaattatagacccaaataaaaataaataaaaagacattctgtgttttagggaaagaagacttaatattgttaagatgtcaatactacccaaagtgatctacagattcaacataatccctatcaaaattccaacagcctactttgtagaaatggaaaagccaattttcaaattcagatggaattgcgaggggttctgaataacaaaaacaatcttggggaaaaaaaacaaaaaacaaagtcaaagaactcacacttctctatttataatttactacaaagttatagtaatcaaagtcgacgcggccgcgattccggg

SEQ ID NO: 45 - DNA, 314 bp, Homo sapiens
  misc_feature (304)..(304): n is a, c, g, or t
cgactgcggctcttcctcgggcagcggaagcggcgcggcggtcggagaagtggcctaaaacttcggcgttgggtgaaagaaaatggcccgaaccaagcagactgctcgtaagtccaccggtgggaaagccccccgcaaacagctggccacgaaagccgccaggaaaagcgctccctctaccggcggggtgaagaagcctcatcgctacaggcccgggaccgtggcgcttcgagagattcgtcgttatcagaagtcgaccgagctgctcatccggaagctgcccttccagaggttggtgaggganatcgcccagg

SEQ ID NO: 46 - DNA, 537 bp, Homo sapiens
gcaatttaatttttaataacaaagatactgtattttaacatggtgaaatatacttggctaagtccagattaaaaaaaaaaagtatctagcccaacagtacaattatacagctttgtacagaacattccatagatcaacagaaaatacatttgagcgcaaaaataaaaaatatttaaggagaatctctaagcagcattttatttctgcaaaagacatatcttgtctgattaaatatctacaagtgcttttcctttcaaaaatacatatattcttaatagactaagtcattaacaatgacctggtaattctttcacttcaatttgaatgatttataagctaaatcttcaaccacaaaaaggtttttatttgtattaagatgttaccacttttgacaaaaagcttaaaatattttatatttcaaaggaaaattagcaacataactttacaatatattctatgatattttgattgtgagggctactctatttaaaactgatgatctctgttgtgttgctcagatgcaggaaagcagcaaaa

SEQ ID NO: 47 - DNA, 534 bp, Homo sapiens
  misc_feature (7)..(7): n is a, c, g, or t
gacttanatctaaatggaccacattctctacttaaaaaaatgctattaaccatgtgatcttctcagtcatgaggtaatctggtgactacccttcctcaaagccagttgggatattctttgaatagagtaaaacagtgtttctaggctgggagacaccagacatagttgaggacagaggtgctagaaaataggaagtttaaaagcatgtgcggtgatgctcagaggaggtaaaccccaccctcatgctcatagcttccaatcattttctctagttcttaactcttaaatgtgagaaatgcttgaagattctagtcatctgaagaaagtctctttattaaagattttcataaaagagaccaaagcagacaaacagaaaaagacatcttggggaaaaaaacaaggataatgggaagagaaggaaagttttaaaaattatcaatatcctcagggggacaaaatattatatcctataaagacagatttttattttttaaaaaaatagaaagcaaaacaagctcctaaaaataaagtttg

SEQ ID NO: 48 - DNA, 444 bp, Homo sapiens
gttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagcttttcctggggacaatgtgggcttcaatgtcaagaatgtgtctgtcaag

SEQ ID NO: 49 - DNA, 566 bp, Homo sapiens
ctttgaagaactttgccaaatactttcttaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtccattccttcaaataaagaaatttggta

SEQ ID NO: 50 - DNA, 400 bp, Homo sapiens
ttttggggtttatatataagcctggttcttgctgaaactgcttatgttgataaccagttagtgagttcctctctattgacttgctgggaagtttatagagacattttttatgcattcagagatttcagtacaaatcttgaaaaagggacatttaggccgggcgcggtggctcacatctgtaaccctagcactctgggaggctgaggtgggtggatcatgaagtcaagagatagagaccatcctggcaaaaattagctgggcgtggtggggtgcgcccgtagtcccagctactcgggaggctgaggcaggagaattgcttgagcccgggaggcggaggtttcattgagccgagatagtgccactgcactccagcctggacaacagagcgagactgtgtctt

SEQ ID NO: 51 - DNA, 562 bp, Homo sapiens
ctaagggtttaaagatggaaagaggcattgatgaacagctggggaaggagtagtttgaggtagatgtgcagatggaatgaagagaaggtctcaagaagagggtggagccaaagagggctgcagatttagaaggctaaagtctttagatggctttggatagcctgttgtatcttggaccatgcaggttacagtggagcatggagtggggacagaagtggaggaaggaaccagggaacatggagtgagaagctaaaggaaagtgatgcagtagatacatggctctaaagtactcaggactttcagaggcttaaacatagggtgaccaactatcccactatgcctgatactaagggcattccctggatgtggacctttcattccccaaattaggaaagtcttgggcataccaagacaagttggccaccctactcaaaagtatgtaagctaacatatctgttctctaagaggttaaagctggatggggataccagatgtatgtacgtgatgcagttaaacagcaatacaagggggcaagtctacctgatcggccaattcaatggga

SEQ ID NO: 52 - DNA, 630 bp, Homo sapiens
gaagccaaaccaaaggagcttctacttcatgatgccatttatgtaaagttcaggcagagaaaatcagtggtttaagaagttagaataatgattatctttggagggattgcaactggaagaagtcatgattgggatttctgggtcctaatagtgctctgtgtcttgatctgagtgccgactacatgagtggttaggtttgcaaaattcattgagttatgcacttaatggtgttgtcttattagagctgatggaggagagagggcttcaatttgcacaactgagtaatcagctaggcccagtcactaggtgaacaacttactgctccaatcagccttagagcaggaatcaaactcatgtctcagaaaagttattaattcagcttgtcttgggacttccttcagagtcactcttgaatagctgaaatagtaaatgttaaatctgtggatgcaagtgtgtaaattattttagtcatcagctctaataagatggcctttggggaaatgagtataaggtcacgaaaatgaaatggcaagaaggaggtctactatttcttctgtaatactgatttttaccccatcagggtcagtccccagaggttgtaaatgtgaagcttgtctttttctttaataa

SEQ ID NO: 53 - DNA, 685 bp, Homo sapiens
ctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaattagggacctgtatgaatggctccacgagggttcagctgtctcttacttttaaccagtgaaattgacctgcccgtgaagaggcgggcataacacagcaagacgagaagaccctatggagctttaatttattaatgcaaacagtcctaacaaaccccaggtcctaaactccaaacctgcattaaa

SEQ ID NO: 54 - DNA, 533 bp, Homo sapiens
cgacccggaattcgcggccgcgtcgactgagttcttgacaagagtgtttttcccttcccgtcacagagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttct

SEQ ID NO: 55 - DNA, 523 bp, Homo sapiens
  misc_feature (462)..(462): n is a, c, g, or t
gaatccctagaaaaagagaattcccaacttgatgaggaaaacttagaactgcgaaggaatgtagaatctttgaagtgtgcaagcatgaaaatggctcagctacagctagaaaacaaagaactggaaagtgaaaaagagcaacttaagaagggtttggagctcctgaaagcatctttcaagaaaacagaacgcttagaagttagctaccagggtttagatatagaaaatcaaagactgcaaaaaactttagagaacagcaataaaaaaatccagcaattagagagtgaactacaagacttagagatggaaaatcaaacattgcagaaaaacctagaagaactaaaaatatctagcaaaagactagaacagctggaaaaagaaaataaatcattagagcaagagacttctcaactggaaaaggataagaaacaattggagaaggaaaataagagactccgacancaagcagaaattaaagatccacatttgaagaaaataatgtgaagattggaaatttggaaaa

SEQ ID NO: 56 - DNA, 566 bp, Homo sapiens; identical to SEQ ID NO: 49

SEQ ID NO: 57 - DNA, 616 bp, Homo sapiens
  misc_feature (571)..(571): n is a, c, g, or t
gacccggaatcgcggccgcgtcgaccattttagccaaggtgcctctataggggtcaagacatcatgtgcccagacctaaggtcaggaatgtcatatttttctgttaaaatcattttatttctgtgtatcttacctttaaatcattgtggtttactctgagattctgtagtcctaatattgtatcattgtgctgtctgcaaaacaacttgaatctattttgtttgcatcttttgttacatgtaacgcagctgtactttatgttctttgcaactgtttccattatgagaacgctgtgctatttacaaggttacatttttcttggccaggcgaggtggtcatgcctgtgatcccagcactttgggaggccaaggtgggcggatcacttgaggtaaagagttgagaccagcctggctagcatggcgaagcccagtctctactaaaaatacaaaaattggccgggtgaaattagccgggcgtggtggtgtgtgcttgtaatcccagctactcgggaggctgaggcaggagaatcgcttgaatccgggaggcagaggttgcagtgagccaagatcangccactgcactccacctcggggtcaagagcgaaactctgtctcaa

SEQ ID NO: 58 - DNA, 598 bp, Homo sapiens
ccgttttagtcaggatggtctcgatctcctgacctcgtgatccgcctgcctcggcctcccaaagtgctgggattacaggcgtgagccaccgcgcccggcgtaaatcaggttttttaaatgtttgccaaaccttatcactgacttttataacaaaattatttactataatcattagggaatatttaagttctgctaatacttaaaattgcagagtgctaaaaccagcagtgagtttagaatcaagctaagctttattgttgctactatttgaggcatattagttgactggtgttcatatgcaaggcagtctactgggtgcaacaagggttagaaggatatttttaaaaaactgaccctattctcaggatgaaaataatacactagtaatagtctgctctgttggttaactcctcgtaaggaggtcaattaaaatgctgtagtgttgcaagggaaggagaggaagaatcatattccttcactagcaggatcaagaaagcttttatagaaatatacaaaatcttcacttcttgaaggattggtaaaatttaatagccaacattgggcacttattcattctctgagtaaatatttattgcat

SEQ ID NO: 59 - DNA, 524 bp, Homo sapiens
cttaaatctaaatggaccacattctctacttaaaaaaatgctattaaccatgtgatcttctcagtcatgaggtaatctggtgactacccttcctcaaagccagttgggatattctttgaatagagtaaaacagtgtttctaggctgggagacaccagacatagttgaggacagaggtgctagaaaataggaagtttaaaagcatgtgcggtgatgctcagaggaggtaaaccccaccctcatgctcatagcttccaatcattttctctagttcttaactcttaaatgtgagaaatgcttgaagattactagtcatctgaagaaagtctctttattaaagattttcataaaagagaccaaagcagacaaacagaaaaagacatcttggggaaaaaaacaaggataatgggaagagaaggaaagttttaaaaattatcaatatcctcagggggacaaaatattatatcctataaagacagatttttattttttaaaaaaatagaaagcaaaacaagctcctaaaaa

SEQ ID NO: 60 - DNA, 534 bp, Homo sapiens
gacccggaatcgcggccgcgtcgacggaagctcctgcccctcctaaagctgaagccaaagcgaaggctttaaaggccaagaaggcagtgttgaaaggtgtccacagccacaaaaagaaggagatccgcacgtcacccaccttccggcggccgaagacactgcgactccggagacagcccaaatatcctcggaagagcgctcccaggagaaacaagcttgaccactatgctatcatcaagtttccgctgaccactgagtctgccatgaagaagatagaagacaacaacacacttgtgttcattgtggatgttaaagccaacaagcaccagattaaacaggctgtgaagaagctgtatgacattgatgtggccaaggtcaacaccctgattcggcctgatggagagaagaaggcatatgttcgactggctcctgattacgatgctttggatgttgccaacaaaattgggatcatttaaactgagtccagctgcctaattctgaatatatatatatatatatatcttttcaccataa

SEQ ID NO: 61 - DNA, 512 bp, Homo sapiens
ggggagccccctcttccctcagttgttcctactcagactgttgcactctaaacctagggaggttgaagaatgagacccttaggttttaacacgaatcctgacaccaccatctatagggtcccaacttggttattgtaggcaaccttccctctctccttggtgaagaacatcccaagccagaaagaagttaactacagtgttttcctttgcaccgatccccaccccaattcaatcccggaagggacttacttaggaaacccttctttactagatatcctggccccctgggcttgtgaacacctcctagccacatcactacagtacagtgagtgaccccagcctcctgcctaccccaagatgcccctccccaccctgaccgtgctaactgtgtgtacatatatattctacatatatgtatattaaaactgcactgccatgtctgcccttttttgtggtgtctagcattaacttattgtctaggccaaagcgggggtgggaggggaatgccacag

SEQ ID NO: 62 - DNA, 642 bp, Homo sapiens
ttttggcattacttaatccaattataaaaactgaatttttaaaaaacagcacttgttttttcttccaagattaatttgaatttttttatggacattagaaaacattgcagtttagtcataatcaaaaataaatcttgaggctggtagagcagctttgttgctgtttatatttttattgcttactggatttcagtgttacctagtgccatcagtttggtattttgccaccttgcacattcagtgatgtttgatttttctttttcctttttttcatattacttttaaatcctgaatagtttgtggcagctggagatcacctagtccaccactgtccaacatggcaatggtaagtaatattgagtaaagaatagaaaattagtaaaatgcatggcttcagaattatagcaatttgcaaaataggttaatggatgaaaattagaatgaccagtttaactttccccccagcagattcttctgttaaacaatgccccttcaaaataaaggaagaacaagtgggtgttatacctatgttatttggctatgttagcacaatatgatggactaatttgagaaaaagcatttacttcctttactattacttcttttctttatagggctaagtctgccttctgggtctttgaa

SEQ ID NO: 63 - DNA, 446 bp, Homo sapiens
  misc_feature (441)..(441): n is a, c, g, or t
gaagaagcgcgaagagccgttagtcatgccggtgtggtggcggcggcggagactgcgggcccgtagctgggctctgcgaggtgcaagaaagcctttgaggtgaaggtgtatgaaagtcatcataacagatgttttccaaaaacttgtagaaggttgtgaaaaaactactaggatcacgcggcatgtattgagcatataggttgctgtagatgaatgttcttagctgtcatgtttaaaaatacttctgcttcgttacctcaagtgtggcatgcagcattttggaaggaaaattgaagacgtgttcaagaaaacatgaacagaagcaaatgatgaaaatgagcattttacttgatgttgataacatcacaataaattatggagaaaaatacatatttggctaacttttaattgctgaacaataaagtgttttcttttaaatcnaaaaa

SEQ ID NO: 64 - DNA, 629 bp, Homo sapiens
gaagccaaaccaaagggagcttctacttcatgatgccatttatgtaaagttcaggcagagaaaatcagtggtttaagaagttagaataatgattatctttggagggattgcaactggaagaagtcatgattgggatttctgggtcctaatagtgctctgtgtcttgatctgagtgccgactacatgagtggttaggtttgcaaaattcattgagttatgcacttaatggtgttgtcttattagagctgatggaggagagagggcttcaatttgcacaactgagtaatcagctaggcccagtcactaggtgaacaacttactgctaccaatcagccttagagcaggaatcaaactcatgtctcagaaaagttattaattcagcttgtcttgggacttccttcagagtcactcttgaatagctgaaatagtaaatgttaaatctgtggatgcaagtgtgtaaattattttagtcatcagctctaataagatggcctttggggaaatgagtataaggtcacgaaaatgaaatggcaagaaggaggtctactatttcttctgtaatactgatttttaccccatcagggtcagtccccaaaggttgtaaatgtgaagcttggtctttttcttta

SEQ ID NO: 65 - DNA, 366 bp, Homo sapiens
  misc_feature (270)..(270): n is a, c, g, or t
gaccctattctcaggatgaaaataatacactagtaatagtctgctctgttggttaactcctcgtaaggaggtacaattaaaatgctgtagtgttgcaagggaaggagaggaagaatcatattccttcactagcaggatcaagaaagcttttatagaaatatacaaaatcttcacttcttgaaggattggtaaaatttaatagccaacattgggcacttattcattctctgagtaaatatttattgcatgcttatcttgtatcaacattgngatgaaagcncaagaatgaaagaggagggagaatgtttanagaataaggctgaaacacagattttgtagggagcgtaggggagactganaaaacag

SEQ ID NO: 66 - DNA, 202 bp, Homo sapiens
  misc_feature (195)..(195): n is a, c, g, or t
aagacacctgatagattgtcttgtattatttttcctttgccttcttacaatctcagtgattagaattgggctgaaaacaatacatcaaattctcagcaaaatccttatgggttgctggataccgagggtttttaagatctttagacttcactatatagaacaaatgttgaatgggaattttctttatttctatancgtttng

SEQ ID NO: 67 - DNA, 634 bp, Homo sapiens
  misc_feature (606)..(606): n is a, c, g, or t
cccggaatcgcggccgcgtcgacgatgagcattttttcatgtgtcttttggctgcataaatgtcttcttttgagaagtgtcggttcatatcctttgcccactttttgatggggttgtttttttcttgtaaatttgtttgagttcattgtagattctggatattagccctttgtcagatgagtaggttgcgaaaattttctcccattttgtaggttgcctgttcactctgatggtagtttcatttgctgtgcagaagctctttagtttaattagatcccatttgtcaattttggcttttgttgccattgcttttggtgttttagacttgaagtccttgcccatgcctatgtcctgaatggtaatgcctaggttttcttctagggttttgatggttttaggtctaacgtttcagtctttaatccatcttttaaaagtctcttcacagtacatgagtagtagtgacaccaataatgtcagagcagggaactcccaggttctgcccatccacaaaaacaacaaataagctggcaaaaactttaagaatcaacttttgcagatctctgaaatctagtcaaaacttaaacagaggaaagattaataaagacnggctgcctgagataacactaacacacac

SEQ ID NO: 68 - DNA, 644 bp, Homo sapiens
  misc_feature (92)..(92): n is a, c, g, or t
catcaaataaataaataaataaattttaaaagtcacagcattgaatttttaaatgtttgggatgataaagcacctgcttatcatgaagctanagaaattcaatgacacgtttgccagggtctttgctagtgatgttggaacaagtctgtaatgctgatgaaacatcactgttcgggcattattgccccagaaagacactgactgcagctgatgaaacagcccttccaagaattaaggatgccaaagaccaaataactgtgctgagatatacttacgcagcaggcatgcataagtgtaaacttgctgttataagcaaaagcttgcgttctcactgttttcaaggagtgaatttcataccaatccattattatgctaataaaaaggcatggatcaccagggacatcttttcagattggtttcacaaacattttgtaccagcagcttgtgcttactgcagggaagctgactggatgatgactgcaagattttgttatatcttaacaactgttgtgctcatcctccagctgaaattctcatcaaaaataatgtttatggctcacacctgtaatctcaacactttgggaggattgcctgacccaggagttcaagcccaccctgggcaacacagcaagacccaacctntc

SEQ ID NO: 69 - DNA, 651 bp, Homo sapiens
  misc_feature (44)..(44): n is a, c, g, or t
ttttaaaaatcataaaacgtttcttacaaaagagcattacattntgcacactgctctgaacagatgccagggacatgtggactattgttacttttcctccctgtcccaccccccaaatgttacagtgaccacaaagcaaggtgttcacaataattacatggggggaattttttaaaccaccaacaataacgaaaaataaaatccactcactctgctgctgtttcaaaatttcaatgttagtttttgcacgcccttcccccccccaaccctgtttgtaaggaactaaaacattacatctggtgaacagcaaagatttcactacacctcaaatgcagaacacctatgaagcagaggaatgttggctttttaaacagaagcagataaaaaaaaaagatgcaggactccttcagttcttcactagtcttagaaaaactttccagaatactgcttcacactataaaaaagaaaaaatatcttgcattagaatccttcaacatctgcatactgcttcacactgttcgtttctaggagcactttgtcacaggacacttctgcttatatttctttaatcagaacttagttggatgggccgggcatggtggctcacgcctgtaatcccagcactttgggaggccgagggggtggatcacc

SEQ ID NO: 70 - DNA, 752 bp, Homo sapiens
  misc_feature (684)..(684): n is a, c, g, or t
ccatctccaaatttagtattcattctgtttagcatattatcagttgccatctatttgttttaactgattacttgaatctgattaaacatcacagaaatgggctttgataagaacaatattgaataagaaattttaaataacaaaacagcttatagaaaaattcagcataacttttccatcaccttcaccacccttgccttttattatcctgtcctgtatcactgctttctgttagcagtgttgtgtgagttaggatttgggcaggaaagcaaaagcaaccacccgtcattttcccagaatgaagggtttgacgtaggatgtagactttgtatagtagttgggagagctgtgggagtgaaggtcagggatgtcacctacagaagtcagggaatctgccaccagagatcctgcatcagaaacagccaacagcgtgcttctgaagaactagtggggaagtggctataattcttaggaatcccagcaagtccgcaccactgtctcagtctacagcagtggagaaaggggtttccaggagctctctggaaagttcctgcccacactttgcaacaatcttcagaggataatgggcttctcttccagcttccacacccaacaagagtgcctttcatcggccaactctaacctggaaccctatggcagaggggatttaggagacagtttgtnatgtctgtggaatgcaaatgaanangtancaatgcttanttgacagcggncatacacaaatntngaaa

SEQ ID NO: 71 - DNA, 12 bp, Homo sapiens
  misc_feature (8)..(8): n is a, c, g, or t
gatccgtngact

SEQ ID NO: 72 - DNA, 505 bp, Homo sapiens
ctcttcccagcccctgagcccagccccttcccaagtggtgccagacaaaaaactacatggccctttcgtgtcttgggggtggaaagggagggatgaattggggtgatagaaccctggtgaattcagagtaatctttctttagaaaactggtgttttctaaagaaacaggataggagtttagagaaggcaccaaagctttcactttggtttggcaccagtttctaaccatctgttttttctaccctagctatcttttattggtaaaatataaatgtataattatgtttgtagagctttaccaaggagtttccctccttttttgtttgttgattagcaaatttttgattctccattttccaaaagtaagagactccagcatggccttctgtttgccccgcagtaaagtaacttccatataaaatggtatttgaaagtgagagttcatgacaacagaccgttttccatttcatctgtattttatctccgtgactccacttgtgggttt

SEQ ID NO: 73 - DNA, 505 bp, Homo sapiens
  misc_feature (280)..(280): n is a, c, g, or t
tggagctgaaaaattcctattacctaggggcatcacaacgcattgcatttcgcccgtgtttgggatgatgctggtgtaaacctactatgctgccagtcatgtaaaagtatagcacacacaattagtaggtaatgcttgcaaataataatgaaagactctgctactggtttatgtatttactatgctatactttttgtcattactttagagtgtactcctactttttttttttttttttttgagatggagtttcactcttgtcctgtaggctggagcgaantggcgcgatctcggcttactgcaacctccacctcctgggttcaagcgattctcctgcctcancttcccagagtagctgagattacaggcatgcaccgccacgcacgggtaattttgtatttttggtagagacagggtttcaccatgttggccaggctggtcaccaactcctgacctcaggtgacccgcctcctcacctccagagtgttgggattacaggngtgag

SEQ ID NO: 74 - DNA, 580 bp, Homo sapiens
  misc_feature (552)..(552): n is a, c, g, or t
ataaaaattagctgggggtgatgggccctgtaccccagctactcgggaggtgaggtaggagaatcacttgaacccgggagatggaggttgcagtgagccaagatcgtgccactgcactccagcctgtgtgacagaacaagactctgtctcaaaaaaaaataataataataataataataaaaaggaataacatagctaggaataaatttaatcaaagaggtgaaagacttatacacttaaaactacaaaaaaaaaatcactgaaggaattatagacccaaataaaaataaataaaaagacattctgtgttttagggaaagaagacttaatattgttaagatgtcaatactacccaaagtgatctacagattcaacataatccctatcaaaattccaacagcctactttgtagaaatggaaaagccaattttcaaattcagatggaattgcgaggggttctgaataacaaaacacaatcttggggaaaaaaaacaaaaaacaaagtcaaagaactcacacttctctatttataatttactacaaagttatagnatcaaagtcgacgcgccgcgatccgggc

SEQ ID NO: 75 - DNA, 535 bp, Homo sapiens
cacagtactccattttggggtccaaactgtaatgctcaaaataataaatgcttacacgaaaattatttattgagaatattcatataaaaattacctaaagcaaagtaaaaaaagtaaaatcaaggtggtatatttgaagtgaatggtgattggaaatttttagctgtaacaaaaagaaagaaaacaactttttttaaagcctcattctcttttctttcaaaatgtaccttattcccacacactcttgggctgacctttattttatcaataagctcaatattactttgtttaaaataagatgcttcagcaaaagtcattctctctttaaccatataatttaaaaactcctcttcacgattgatagcaaaatcagaaacgttagggcaccagtgagttgaaaaaactggtcttaagttggaaaaactattattaataatattatcctatccatccatatctattgaaattgtcaggtccataatttcattttaattaattataggaaagaagaaaagataatacccatttgttctat

SEQ ID NO: 76 - DNA, 505 bp, Homo sapiens
  misc_feature (461)..(461): n is a, c, g, or t
ctcagactctttctgccctaatggccattactatccagtctgtattgctacaagggacccactggtaccccttttagattctatcaaaaggaacagggttttcctagaggcaggcagcctggtggtatggcacagcagaagcttactgctaatgaaatgggaacctccccctcccttgtggtttcagcacagaacctgaatgccaggaaaaattcctgggccaagaagctaaagctaaagaaaccttccttttttcaacgtttttttttctttcaaactgtagggtcacttttgattgaggcaaaggggtcctactgtaagtggaaaagactcactcccctaacataagttttcactgtggtgggatggtgccgcccgatatgcttgatatgcttttccttccacatgttaagctaggaaacctaacaggatgtcagcagggcagttaactctggactcanagccctcaagggcatgtggcanaacctcatggcatncaagacca

SEQ ID NO: 77 - DNA, 596 bp, Homo sapiens
  misc_feature (567)..(567): n is a, c, g, or t
gtataattgattcttttgaacctaaagtataagacttcacgattagaaaaaaattatccaaagactaatgtaattaagtgaggaaaaggtgctggaggaactggataaccacatggaaatgtatgaaccatgacctctatgtcacatactatatataaaacttaatttgaggtgtatcacagagctaactgtgggggctaaaacgttgaagcctttggatggccgcacaagagatgtctgcattcataaccttggggagggtatgaacatttcttggtaacatgcaaaaagcactaactgtaaaagagaacagttggtcagttgaatttcatgaaacattgtaaacttctgctaaacaactgacaccattaagaatgtggaaaaaggctgggcacagtggctcatgcctataatcccagcattttgggaggccggggcgggagaatcacttgaggccaggagtttgaaaccagcctgggcaacatggcaagaccccgactctacaaaaatatttttaaaaattagttgggtgtggtgatgcactcctgtagtcctagctgccaggangctaaggnggaaggatcacttaaccctgg

SEQ ID NO: 78 - DNA, 504 bp, Homo sapiens
ctggtggcggcggtcgtgcggacgcaaacatgcagatctttgtgaagaccctcactggcaaaaccatcacccttgaggtcgagcccagtgacaccattgagaatgtcaaagccaaaattcaagacaaggagggtatcccacctgaccagcagcgtctgatatttgccggcaaacagctggaggatggccgcactctctcagactacaacatccagaaagagtccaccctgcacctggtgttgcgcctgcgaggtggcattattgagccttctctccgccagcttgcccagaaatacaactgcgacaagatgatctgccgcaagtgctatgctcgccttcaccctcgtgctgtcaactgccgcaagaagaagtgtggtcacaccaacaacctgcgtcccaagaagaaggtcaaataaggttgttctttccttgaagggcagcctcctgcccaggccccgtggccctggagcctcaataaagtgtccctttcattgactggagcag

SEQ ID NO: 79 - DNA, 564 bp, Homo sapiens
gcaggggcttctgctgagggggcaggcggagcttgaggaaacccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagaccaaaatggaaagattaattgggagtggtagga

SEQ ID NO: 80 - DNA, 270 bp, Homo sapiens
cccggaatcgcggccgcgtcgacgggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttataaaaaa

SEQ ID NO: 81 - DNA, 268 bp, Homo sapiens
cccggaatcgcggccgcgtcgacgggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttataaaa

SEQ ID NO: 82 - DNA, 334 bp, Homo sapiens
cttcatttgaaatggttgaatctgctgtgtaataaagtggttcaaccatgattaggaactgaaatttagtagaagagggaaaaggagttaatgtaacaaattattttagctacaaaccccggtaatagagcacttgggggatgggatggggtgggttggtgagacaatcagaatggtaaattgattaaatgctcctaaccctgtaattttgtgcatagagcaccctatgctgtggaaataactgttcttagatttcattgtaactggactgttcaggttgcccagagggaaagaacattcctaattctaataaaataaacttttattttgttta

SEQ ID NO: 83 - DNA, 675 bp, Homo sapiens
tgtcattgaatctgcttgttacttaaatgctaaactcaattctgtaattcaataggtgcacctctctgagaaacataagagacaatgaggaaaaggattcagcattccgtggaatttgtaccatgatcagtgtgaatcccagtggcgtaatccaagtaagatgttcacaaagatttgtttttaatgtctaattaataaaattttaaaggaagaaacattctaatactttaattataaaaagttaactattttcaaaggtatcaaaatacagttaaacctttaaaatgtatatttcttaatatcttgaaattgtaatgccttttttttttcctaaattttttttgtcatgaaatgagatagtaacagcagattgggacaacaaggttatattcttgtcttgaatcaggccatggcttctttcatccaaatttcagacctcatttatttactttgtccctgcctcccatccctggatatcagtttgtggatatctacagttaatagagtgaccaaatagtaggaatactgtctctctattctgaataaaatctttgaatcagatttagaaataatgaataaaatacaaatcagccattgaaattgctctaattttgagagcttatgatttattcatctttggtttccaagttcaagttatatgtagacattttaatt

SEQ ID NO: 84 - DNA, 485 bp, Homo sapiens
  misc_feature (223)..(223): n is a, c, g, or t
gcttcctaggtgaggtcacgaggaaacctgctggccaagtgacctggcagggtgtggccagtgtggccagggccgccgagcctgctttccttccctgcagcaggaacccttctggggctgtgatcctgcgatggtgcctgggtgggagtgggggtggggggcgggatggtctccctacctgccagcttcttggtttgaggtgaggacagccccggaagctcanacttggctcctgtccatgtacttggggccatgagctctgcagggaccttggaaaganagagacgggtggtgtanggcangggaaggcattgtcttcaaacaggaaaaagctganaatggaaacaggcgaaacttaccaagtgtaacatcacctggaactgaaggagggtgggaaggttttaattattttaaaaatagagatggggtctcactatgttgcccaggctggtctcaaactactgggctcaagtgaacctccttct

SEQ ID NO: 85 - DNA, 387 bp, Homo sapiens
  misc_feature (9)..(9): n is a, c, g, or t
tgtttctcnagggcgagaggctgtcttanagcaccattctctggccctngtcccatgagaaggaaccgcactcaggagccacactctcccactncccttgcccanaagactcacagagggcacggagctggctgtggtgagaggaggtccancaaattcctgtctgcanaagggttctgaacaccaccgcctggcagcgtgctggaggagggattcctcttttcctcacagcaattctgaccagaaacctgtcaaatcaggaatggctaaaataagaccagggtatgaatgaccatcagccacagtaaaaccaaggcacagctctcctgagcccacccaagctgctgtggcccagactggtgacatcacctcagggcaaaaaaaaaa

SEQ ID NO: 86 - DNA, 420 bp, Homo sapiens
cgcagaatggctcccgcaaagaagggtggcgagaagaaaaagggccgttctgccatcaacgaagtggtaacccgagaatacaccatcaacattcacaagcgcatccatggagtgggcttcaagaagcgtgcacctcgggcactcaaagagattcggaaatttgccatgaaggagatgggaactccagatgtgcgcattgacaccaggctcaacaaagctgtctgggccaaaggaataaggaatgtgccataccgaatccgtgtgcggctgtccagaaaacgtaatgaggatgaagattcaccaaataagctatatactttggttacctatgtacctgttaccactttcaaaaatctacagacagtcaatgtggatgagaactaatcgctgatcgtcagatcaaataaagttataaaattg

SEQ ID NO: 87 - DNA, 675 bp, Homo sapiens
ggaaactgatgccagtcagaaactcagatcaaatgaaggggtgaagagaaccagaattgatctctctgtaggagaatataaatgacttttttaaagtacatattttctgtgaaagacagttttttgtttaatgcaaaaatgttaacaatgtttatatcatgtagaagtaaaagatcgtgaaacagcacagagaacagtagtaagacagattgaattgcactgttgtaagatgatgaacttacaatattaagtgaaggtagactgtgatagattaaggatatatattgtaatccctagagcaattgtcaaagtggtacaggtaaaaagccaatagaggtgataaaatggaatactaaaaaatatcagatgaataataaagaagacaggaaatgaggaacagtggaacagaatgaataaaaaacaagaccattaacttaatcattaataattactttaaatgggttaaacattatggttataaggcagagattttcagactagataaaagagcaagctccactatatactgtctacaagagatatactttaaagtgtatattatatttaaatataaagatttggaataaataaacctaagaataagcttactagggaagtgaaagatctgtacaacaagaattacaaaacactgctgaacgaaatcataggtgacca

SEQ ID NO: 88 - DNA, 594 bp, Homo sapiens
  misc_feature (529)..(529): n is a, c, g, or t
gtcccggaatcgcggccgcgtcgacgtttcctcaaaatttatcttcctgttaatgtcaggcatgtatctccttagcttgccacaaataactatatataccacagaccttcctttgtagggctaacagtgttgcattgtaagtggaggcctcatagatacctggccttttcctaccttattccaaagatggttgcatcttataaataatgtcattcttcagcaaatggtatggaaatgagattgtaatgtcattatttcctctttaaataatcaggacaactcatgatacaaagagctcttctctataaaaggtgggacttttttttttagtaatagcaaaaataaaattgtacctccttaatcttctacagaaagatggatttcattttcaacattaagaggtagttttaagaagcagtagaagtcagcctgggcagcatggtgaaaccccgtctctacaaaaaagttagctgggcttagtagttgcaatcccagctactctggaggctgaggttggagatcatctgancctggggaggtcnaggctgcaatgatacantgagccctgattgtgccactccacctggttgcaga

SEQ ID NO: 89 - DNA, 530 bp, Homo sapiens
ttccaatcttcgtgttcactttaagaacactcgtgaaactgctcaggccatcaagggtatgcatatacgaaaagccacgaagtatctgaaagatgtcactttacagaaacagtgtgtaccattccgacgttacaatggtggagttggcaggtgtgcgcaggccaagcaatggggctggacacaaggtcggtggcccaaaaagagtgctgaatttttgctgcacatgcttaaaaacgcagagagtaatgctgaacttaagggtttagatgtagattctctggtcattgagcatatccaagtgaacaaagcacctaagatgcgccgccggacctacagagctcatggtcggattaacccatacatgagctctccctgccacattgagatgatccttacggaaaaggaacagattgttcctaaaccagaagaggaggttgcccagaagaaaaagatatcccagaagaaactgaagaaacaaaaacttatggcacgggagtaaattcagcattaaaataaatgtaattaaaagg

SEQ ID NO: 90 - DNA, 381 bp, Homo sapiens
tgcaggatccgtcgactctagataacatggctagaaaagagaatgaaaaagttggaatttttaattgccatggtatggggggtaatcaggttttctcttatactgccaacaaagaaattagaacagatgacctttgcttggatgtttccaaacttaatggcccagttacaatgctcaaatgccaccacctaaaaggcaaccaactctgggagtatgacccagtgaaattaaccctgcagcatgtgaacagtaatcagtgcctggataaagccacagaagaggatagccaggtgcccagcattagagactgcaatggaagtcggtcccagcagtggcttcttcgaaacgtcaccctgccagaaatattctgagaccaaattt

SEQ ID NO: 91 - DNA, 535 bp, Homo sapiens; identical to SEQ ID NO: 75

SEQ ID NO: 92 - DNA, 619 bp, Homo sapiens
caggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacc

SEQ ID NO: 93 - DNA, 131 bp, Homo sapiens
  misc_feature (70)..(70): n is a, c, g, or t
cgctgggtgcctgcagcgcctcccttgtctcatatggtgtgtccagcactctattgttgtaaactgttgntttgnctgacctaaattntctttactaaacanatttaatagttnaaaaaaaaaaaananca

SEQ ID NO: 94 - DNA, 607 bp, Homo sapiens
ttttaaagtcatctctataggaaggtgctgggcagggatcccagagaaagaaagggtccaagactccattaactgccctggatgaagggcactgctacagcagctagtaccagagactctcctatctcacggttgaggcagacccaggatagaatagagaataaaaggaatgcttataggaaacaattttgtatggaatgctagatggccaagcctcagcctttggtccagtgcaacccttgcctcgcttgtcaacagtgaaaaattagtttggttagaagaaccatctggaaacacaccagcttctgctaccttcatgctcattgttaaaaaaagattaaccagtgtgaacattctgatctgttaattccagggactgttttctttccaatggactgtttgttggtagaataacccccaaaagctcaaagctaaaatgcatcatcagtcctagtcggcagttccttaagaatggactggcggcgtggttgagctgatatggaaaagctgcaccttcctgcagaagatcaactgacctgctatcccaccccaaatttcaacctgaggtatatttcaatgaaggcaggtagctgtgcttctcagagca

SEQ ID NO: 95 - DNA, 687 bp, Homo sapiens
  misc_feature (650)..(650): n is a, c, g, or t
tcccggaatcgcggccgcgtcgacccgccgccgaggattcagcagcctcccccttgagccccctcgcttcccgacgttccgttcccccctgcccgccttctcccgccaccgccgccgccgccttccgcaggccgtttccaccgaggaaaaggaatcgtatcgtatgtccgctatccagaacctccactctttcgacccctttgctgatgcaagtaagggtgatgacctgcttcctgctggcactgaggattatatccatataagaattcaacagagaaacggcaggaagacccttactactgtccaagggatcgctgatgattacgataaaaagaaactagtgaaggcgtttaagaaaaagtttgcctgcaatggtactgtaattgagcatccggaatatggagaagtaattcagctacagggtgaccaacgcaagaacatatgccagttcctcgtagagattggactggctaaggacgatcagctgaaggttcatgggttttaagtgcttgtggctcactgaagcttaagtgaggatttccttgcaatgagtagaatttcccttcctcccttgtcacaggtttaaaaacctcacagcttgtataatgtaaccatttggggtccgcttttaacttggactagtgtaactncttcatgcaataaactgaaaagaccatgctgctantc

SEQ ID NO: 96 - DNA, 462 bp, Homo sapiens
  misc_feature (204)..(204): n is a, c, g, or t
ttcggacgcaagaagacagcgacagctgtggcgcactgcaaacgcggcaatggtctcatcaaggtgaacgggcggcccctggagatgattgagccgcgcacgctacagtacaagctgctggagccagttctgcttctcggcaaggagcgatttgctggtgtagacatccgtgtccgtgtaaagggtggtggtcacgtggcccanatttatgctatccgtcagtccatctccaaagccctggtggcctattaccanaaatatgtggatgaggcttccaagaaggagatcaaagacatcctcatccagtatgaccggaccctgctggtagctgaccctcgtcgctgcgagtccaaaaagtttggaggccctggtgcccgcgctcgctaccagaaatcctaccgataagcccatcgtgactcaaaactcacttgtataataaacagtttttgagggattttaaaa

SEQ ID NO: 97 - DNA, 535 bp, Homo sapiens
  misc_feature (248)..(248): n is a, c, g, or t
ctgcaatgtgcaatagttgcaccactgcactccagcctgggtgacagagtgagaacctatctcttaaaaaaaaaaaaaaaaaaaggaagaagagacatgagagggcccaagtcacttgctcactcactttccgtgtacatgtaccaagaaaaggccatgtgggaaagagcaagaaggcagccgccttcaagacaggaagagagccctcaccagaaactgagccagaaccttggaattccagcctccanaactgtgagaaaagaattttctgttgtttcagtcccccacactatggcattttgttacggcagcctgagctaatactcctactttgtcctgcatttacttggtcttccagttagttttttagactttgggaatcagagcagtcagttgtcagattttagcttacagttgtcctacctgtgcaactgaaatttcttccattttaaaccagagcagagttttagagtcaaaagaaaccagatcttttagtgcagaagctttccactgtattanaagtgaggaagttggt

SEQ ID NO: 98 - DNA, 512 bp, Homo sapiens
  misc_feature (425)..(425): n is a, c, g, or t
aaaaaaactccagagaagtttatagaaagagatgacatgtaaaccctgctgaaaaatagtttcatttgttagaatataattgtcttccactaaaaaaagaaaaaaaaaagcatttaaggctctaagatctcttgaagtaccacttttcctgaatcccagagtttttatgtgcattatttttatgcgtttgtagtttgatatgttgtatttataagtagttttagctttccattatgaattcttctttgacccatgagttatttaggtaagtgtttaaaaatttacaatagtttatatatgcaaatattatgttgttagagttggttttcatgtcatttttacatatacaggggcagtttccccaactaaattgtatattccttaaagcagcactcttaaattttatttctgtgtcaatttcttgnctgtgtttcctggcatggaatacatggcataaaatttgttatgtaattaaatgaaatattattatactttctattttttagaaaaaa

SEQ ID NO: 99 - DNA, 577 bp, Homo sapiens
  misc_feature (552)..(552): n is a, c, g, or t
gtcgacagggatgacataactattagtggcaggttagttgttggtcactttcaactctgggttcaagcgattctcctacctcagcctcccgagtagctgggattacaggcatgcaccgccacacctaattttctattcttagtagagacggggtttctccctgttggtcaggctggtctcgaactcccgacctcaggtgatctgcctgcctcagtctcccaaagtcctggaaccacagacatgagccaccacgcctggccccttttaaaatatttctgctcattgatgatgcacccagtcacccaagtgctctgatggagatgtataaggagatgaatgctgttttcatggctgctaatacaacattcattctgcaacccccaaatcaagaagtaattttgactttcaagtcttattatttaagaaatatattttgcaagactatagctgccatagaccgtgattcctctgatggatcagacaaactaaaatgaaaacctcctgcaacgtattcatcattctagatccctgaggaatcgccacactgacttncacaatgggtgaactgggttacagt

SEQ ID NO: 100 - DNA, 552 bp, Homo sapiens
aaacaaaattattctctgagagggaaaggacatttgagggaaacatcaaatttccccataaataaatgaatggagtttgcaggaaggtgagggtgagcagagatgtgtgtggacatctctgaccatccatcgctgtattcaaatggattgttttattccattctggtctcaggcatgaccacgtccagtgaagacatttgaggcagcacatctcaggacccaggcaatagactggccccaactcaggctggactaaggtgtgattaattctttgttttttgtgtggaacagctcaccttgtcagacagcctcagggcatctctgagacacaggggcagaaaatgacattcatcttttgagtcctcatccatggagtgctgtgtttggggggctgcatctgctgaagcgagaaccccattctgccaccccaccaggatgcccattctccaggacttctccaacttactattagactaaaccagaacaagcaacaaactgtatttatgcaagcaaaattgatgagaaaattatattcaaataaagcaaaaatta

SEQ ID NO: 101 - DNA, 606 bp, Homo sapiens
  misc_feature (565)..(565): n is a, c, g, or t
tcgtgccactgcactccagcctggacgacagagtgagactccatctcaaaataaataaataaataaataaataaataaataaataaaaaaataaaaaatacttctgctatgaaaaacctagttggtatttttgcttatttaatactatagaaatatggtgatctcatctttaatagagtgcttttaaggtccccagtgataatctcctaaaatcatgaactttaagaatttataatgttaatatgaggaaatgaaatctggattatctcaccacatattatataattcattagtgacagagcaagaactccaggtcacctgtctattccatgtttttcctatctgcctttaaatgttgagatactacccttatctcatgtgaatggagaaactgcctaaaatgctaaaactgactcagaggcacccagacataagtgaagtgtgattagaaaatcctggtcagttgagtcttagccaaatgtgtacctactgtgtctgcctctatcaagtcaatgaaaacatgatctgagaactgtaagtccatttatggaaagggttgatttanagatattttgaacttncagtgatgagccccttctcaaatag

SEQ ID NO: 102 - DNA, 341 bp, Homo sapiens
  misc_feature (86)..(86): n is a, c, g, or t
cggactcctgtgctaattgtcagcttacatatcattgtatagagactgtttattctgtaccaaactgatttcaaaagtactacatngaaaataaaccggtgactgtttttcttcataaagttctgcgtttggcatcttcactctttccaaaatgtatctgtacatcanaaatgtcactattccaagtgtctttttagtgtggctttagtatggcttccttttaatattgnacatacattgnatctttgttttatggnaataagtaataaaaatgtagacttcatattttgtacaaaatgtcctatgtacagaataaaaaagttcatagaaacagccnanaa

SEQ ID NO: 103 - DNA, 647 bp, Homo sapiens
  misc_feature (20)..(20): n is a, c, g, or t
aggccgaggcaggcagatcncntgaggtcaagagtttgagaccagcntagctaacatggtgaaaccccatctctacaaaaatataaaaattagcctgggtggtgatgggcacctgtaaccccagctactcgggaggctgaggtaggagaatcacttgaacccgggagatggaggttgcagtgagccaagatcgtgccactgcactccagcctgtgtgacagaacaagactctgtctcaaaaaaaaataataataataataataataaaaaggaataacatagctaggaataaatttaatcaaagaggtgaaagacttatacacttaaaactacaaaaaaaaaatcactgaaggaattatagacccaaataaaaataaataaaaagacattctgtgttttagggaaagaagacttaatattgttaagatgtcaatactacccaaagtgatctacagattcaacataatccctatcaaaattccaacagcctactttgtagaaatggaaaagccaattttcaaattcagatggaattgcgaggggttntgaataacaaaacacnatcttggggaaaaaaaacaaaaaacaaagtcaaagaactcacacttctntatttataaatttactacaaagttatagtaatcnaa

SEQ ID NO: 104 - DNA, 329 bp, Homo sapiens
tacgcacacgagaacatgcctctcgcaaaggatctccttcatccctctccagaagaggagaagaggaaacacaagaagaaacgcctggtgcagagccccaattcctacttcatggatgtgaaatgcccaggatgctataaaatcaccacggtctttagccatgcacaaacggtagttttgtgtgttggctgctccactgtcctctgccagcctacaggaggaaaagcaaggcttacagaaggatgttccttcaggaggaagcagcactaaaagcactctgagtcaagatgagtgggaaaccatctcaataaacacattttgggttaaaa

SEQ ID NO: 105 - DNA, 504 bp, Homo sapiens
gagcagtggcatgatcacaccttactgcggcctccaacccctgagcttaagtgattctcccgcattatcctcctgagtagctgagactacaggtgcatgccaccatacactactaaatttgggtcgggtggtggtggtgattttttaatatttttgtagagacagggtctcactgtgatgcccaggctggtcttgaactcctgggctcaagcagtcacccacctcagcctcccaaagcactgggattacaggtgtgagccaccacactggccagctttgttttgttttgatgactaagctgctcttgctaaaagggcttctctctgaacttccctacctttcttctgtttccctgggctagggctccatgttggcagtcctactcccaattaacctggggctgtctggttaacctttataagatctgcagtcattgggagacccggggaccaggaatattgttgttgagggagctaccctggaaagtggatgggtggccaaagg

SEQ ID NO: 106 - DNA, 582 bp, Homo sapiens
tttggctttgcctctaggcattagatgttatctttggaggcatccttctatgagcattcatttttggaccaagcctggatttacaattctattactggcccagacttcatttctatccaatttcattccactgtgctatagtttacaacatataatttgacttataaataattcctgactatgggtttaaagactgaaaatggatcaatagaaactttgaaaatgttaacatcttgattgcttttctcagtgtagaaatggacaatgtttagcttaaaaactgcatgtttttaatgagatacggggttgaaagacttattcctggaatttattgttctggagaaagcctgttgctatctgccataccttggtttactttgtgcaaaatgagcttctttttaagtaatgagctctttccatgttcagcttaaattgctgtcttagacacttcatcagggttccctgctctgcctcattcccccttttgctcacttgcagcctttgacataatcctgggaggcaattggcatcatacatattttgctttgtaatctcctgctttgattctgactgggacccagc

SEQ ID NO: 107 - DNA, 747 bp, Homo sapiens
  misc_feature (708)..(708): n is a, c, g, or t
ggatctaagaccagcctggcagccaccagatggtgattctagtcctggctcagtcagtaataggtcactgaccccagagaaatcaattcagcctccccaggtccttggatttctttctgtgaaaatgaaagcataggtaggaatttcccatggaacagctagcagaggagaaatattaaaagtcaggagactcatgctatagttttcatacttcattacaacaatgttgtttaggacaagtgagttaacctgttagcttcctctatataaaatggaaagtcattaaaaacctacatagcagggttcttgtgaagatcaagtgataatgtaggaagcatgtacaaatgtcacattctgccgtcacgtaatggtcctcacagcttgaggtagcatttagcatgtgtcatgatttagtacaagggttggcaaactgttgctcttggattaagtctggctcattgcctgtttttcaaagaaaaaaattgtatatgtgtgtatatatgttatatataggtacacacacatatgtgctatatatagcatatatacacacataatatataaacatgtacatatatagcattatatatataccgtgtataatatctccagtcctcatgaccagccatgcttgttcatttacatttgcatactctatgattgctttcatgcaacaatggcagagttgagtgattgttttgcacaganactgtatggcccactaaacctaaaatattaatctctgcc

SEQ ID NO: 108 - DNA, 522 bp, Homo sapiens
ctcctgccgggctcgtggcggcttctgtccgctccgcggagggaagcgccttccccacaggacatcaatgcaagcttgaataagaaaaacaaattcttcctcctaagccatggcatatcagttatacagaaatactactttgggaaacagtcttcaggagagcctagatgagctcatacagtctcaacagatcaccccccaacttgcccttcaagttctacttcagtttgataaggctataaatgcagcactggctcagagggtcaggaacagagtcaatttcaggggctctctaaatacgtacagattctgcgataatgtgtggacttttgtactgaatgatgttgaattcagagaggtgacagaacttattaaagtggataaagtgaaaattgtagcctgtgatggtaaaaatactggctccaatactacagaatgaatagaaaaaatatgacttttttacaccatcttctgttattcattgcttttgaagagaagcatagaagagactttttatttatt

SEQ ID NO: 109 - DNA, 682 bp, Homo sapiens
  misc_feature (600)..(600): n is a, c, g, or t
tgccactgaagatcctggtgtcgccatgggccgccgccccgcccgttgttaccggtattgtaagaacaagccgtacccaaagtctcgcttctgccgaggtgtccctgatgccaagattcgcatttttgacctggggcggaaaaaggcaaaagtggatgagtttccgctttgtggccacatggtgtcagatgaatatgagcagctgtcctctgaagccctggaggctgcccgaatttgtgccaataagtacatggtaaaaagttgtggcaaagatggcttccatatccgggtgcggctccaccccttccacgtcatccgcatcaacaagatgttgtcctgtgctggggctgacaggctccaaacaggcatgcgaggtgcctttggaaagccccagggcactgtggccagggttcacattggccaagttatcatgtccatccgcaccaagctgcagaacaaggagcatgtgattgaggccctgcgcagggccaagttcaagtttctggccgcagaagatccacatctcaaagaagtggggcttcaccaagttcaatgctgatgaatttgaagacatggtggctgaaaagcggctcatcccanatggctgtggggtcaagtacatccccaatcgtggccctctggacaagtggcggccctgcactcatgaaggctttcaatgtgc

SEQ ID NO: 110 - DNA, 668 bp, Homo sapiens
  misc_feature (616)..(616): n is a, c, g, or t
tcccggaatcgcggccgcgtcgaccttgtccttgagcgtcaaccttctttccctgaagtggctggggttcctgtttccttctttgattgacaacttgtgttaaccctcgcacatctctgggccaatttttgcttgtaagtctttccggagacccctggaatttaaatcattagcaccgcgcccttccccgaagagtcttcgaagggttgccgcttttcggtggcgcagttctcgcgagaaggtgactttctttctcggtatttcctggtttccagaatccttagcgcgaggcggaaaaaatatttctcccagcttgtgttgatgccgcgattttgactgagacttcttcccacgatttctgtttttgcttctccaaggaaaatggcagctcccgagcagccgcttgcgatatcaaggggatgcacgagctcctcctcgctttccccgcctcggggcgaccgaacccttctggtcaggcacctgccggctgagcttactgctgaggagaaagaggacttgctgaagtacttcggggctcagtctgtgcgggtcctgtcagataaggggcgactgaaacatacagcttttgccacattccctaatgaaaaagcagctntaaaggcattgacaaactncatcaactgaaacttttagtcatactttaatcg

SEQ ID NO: 111 - DNA, 536 bp, Homo sapiens
  misc_feature (452)..(452): n is a, c, g, or t
cagagatcaaaataggccttacacagtgcgacgcgaatttaaaagattaccccattcaggtgtatggattttgcagtattaaagatgctgcctggaataggtcattatcttctccaagtactctgttaagtcaatgagtcacatagagtataaggtttattatctgcttttctttcattaaataaatctttattgaatttctactacattaaaaaaccaaaccaaaacaaaacaaacaaaaaaaacacttccctgagccataaaggagaaggtagttttgactggaaccttgaaggatgggtaaactttcagcagataaagattgagagaagaccttccaggtagagaaagcagtgtgggcacaggcaaagatggaagaacacacgtggctgtgggaaacacagctagaagccagtgcggatagagagtaggctatgatgtgcaaaggttanacactgggagagacaggtccatgagagtagcttggactaacacagggagggtttggaatcccaactggggaacctanaaatcaa

SEQ ID NO: 112 - DNA, 368 bp, Homo sapiens
taggaggcttattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaat

SEQ ID NO: 113 - DNA, 338 bp, Homo sapiens
  misc_feature (318)..(318): n is a, c, g, or t
tctttatcaagttgagaaagttcctcccctctattcctagtttgctaagagtccttctatcctatttcttaatggtttagtagatgactctgtggtactttgaaggttgtttgcagaatttccatgccataggcaatttacctttccttgacatttgaaggattgatgttggtgccaagtatagaatcttcacagagtcctcctgtagcttctaaaggtttagcttgaaaatgttaattgcttaacgctagtaagtgagtgaaaaagctggggataaattttgtatcttgcttatatttcagttcccacctctgtccngacnaaacccccatatataa

SEQ ID NO: 114 - DNA, 383 bp, Homo sapiens
  misc_feature (236)..(236): n is a, c, g, or t
tagtttacatatcccaacctttaaaaatattcctcttattagctttatattcactttatagaagttgagttttaattaaaattcttggcatcctgaagtatgtcacatagcatgtgctccttataaatatgttgatatctcagaagacagcatcccggttttcattttataaagtaccatacttaagaatgctgtaatacttatcttttataacatgtttccttcgctttgcttgncttttatgncatcagttttaactgtttacttcatttaacagnttacatcatncaacagtttacttcattaaacagtaggtggaaaaatagatgccagtctatgaaaatcttcccatctatatcaaaatactttcaaggatatacttt

SEQ ID NO: 115 - DNA, 615 bp, Homo sapiens
  misc_feature (586)..(586): n is a, c, g, or t
cgactttcaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaacctatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgagggtgcaacaccccattttcccttctggagacttctaatgaaacanatttcctgattggcattaatgaanagtca

SEQ ID NO: 116 - DNA, 161 bp, Homo sapiens
  misc_feature (144)..(144): n is a, c, g, or t
gatttttaaaaatacatatagcaaaaatattacagggtcaggggagacaattagaatgatataattcaaagtggattaaaaaaaaaactgtcacccagaatacaatacccagcaaagttgtccttcataaatgaaagaaaaatnaaatctttnccnaacna

SEQ ID NO: 117 - DNA, 28 bp, Homo sapiens
tcccgggaatctgcaggatccgtcgact

SEQ ID NO: 118 - DNA, 258 bp, Homo sapiens
  misc_feature (28)..(28): n is a, c, g, or t
gacagtgcccagggctctgatatgtctntcacancttgnaaagtgtgagacagctgccttgtgtgggactgaaaggcaagatttgttcctgcccttccctttgtgacttgaagaaccctgactttgtttctgcaaaggcacctgcatgtgtctgtgttcttgtaggcataatgtgaggaggtgggganaccaccccacccccatgtccaccatgaccctcttnccacnctnacctgtgctccctccccaatcatnttt

SEQ ID NO: 119 - DNA, 694 bp, Homo sapiens
  misc_feature (85)..(85): n is a, c, g, or t
tgggctttgggctggctgcagtctgtctgagggcggccgaagtggctggctcatttaagatgaggcttctgctgcttctcctagnggcggcgtctgcgatggtccggagcgaggcctcggccaatctgggcggcgtgcccagcaagagattaaagatgcagtacgccacggggccgctgctcaagttccagatttgtgtttcctgaggttataggcgggtgtttgaggagtacatgcgggttattagccagcggtacccagacatccgcattgaaggagagaattacctccctcaaccaatatatagacacatagcatctttcctgtcagtcttcaaactagtattaataggcttaataattgttggcaaggatccttttgctttctttggcatgcaagctcctagcatctggcagtggggccaagaaaataaggtttatgcatgtatgatggttttcttcttgagcaacatgattgagaaccagtgtatgtcaacaggtgcatttgagataactttaaatgatgtacctgtgtggtctaagctggaatctggtcaccttccatccatgcaacaacttgttcaaattcttgacaatgaaatgaaactcaatgtgcatatgggattcaatccccaccatcgatcatagcaccccctatcagcactgnaaactcttttgcattaagggatcattgc

SEQ ID NO: 120 - DNA, 337 bp, Homo sapiens
ggcagcgcggggagcccgtcggcgccggcgggcgggccggtttcgaagttgatgcaatcggtttaaacatggctgaacgcgtgtgtacacgggactgacgcaacccacgtgtaactgtcagccgggccctgagtaatcgcttaaagatgttcctacgggcttgttgctgttgatgttttgttttgttttgttttttggtctttttttgtattataaaaaataatctatttctatgagaaaagaggcgtctgtatattttgggaatcttttccgtttcaagcattaagaacacttttaataaacttttttttgataatggttaaaaaaaaaaaaaaaa

SEQ ID NO: 121 - DNA, 210 bp, Homo sapiens
cataataaaaaacaatcaacaaacagggaatggaaagaaacttcctcagcatggtgaaggccacatatgaaaatcccacagctaacatcatactcaatgatgaaagactgaaagcttttctcctgagatcaggaacaagacaaagatgtcaccttttgtcacttctattcaactcattattggaagtttttgccagagcaattaggtaag

SEQ ID NO: 122 - DNA, 476 bp, Homo sapiens
cagaatcttttcataggctgaatgttgctccacaatgtgtcctttgactatctctggctaattattattttaatctcttctcagcttttccaagaacataacgttaaccaaagatcttaggccattcacaactcttttgtaaaaattaatgtggatgtgaaacgaggcaacaaatcctgaagtagaaagttattcctggccaggcacggtggctcacgcctgtaatcctggcactttgggaggccgaggtgggtggatcatgaggacaggagatcgagaccatcctggccaacatgatgaaaccccatctctactaaaatacaaaaaattagctgggcatggtgacgcgtgcctgtagtcccagttactcgggaggctgaggcaggggaattgcttgaacctcggaggtgggaggttgcagtgtgccgagatcacgctactgcactccagcctggcaacagagcaagactccatct

SEQ ID NO: 123 - DNA, 250 bp, Homo sapiens
aaacagaaagtttcttctaaaggcatgattcagttaagtcattcttaagtgttaaaaaattgtgaaaaatgtgcctgtaatcccaacactttgggaggccgaggcaggcagatcacgaggtcaggagatcaagaccatcctggctaacaaggtgaaaccccgtctctacgaaaaataccaaaaacattagccgggcgtggttgtgggcgcctgtagtcccagctacttgagaggctgaggcaggagaatg

SEQ ID NO: 124 - DNA, 523 bp, Homo sapiens
  misc_feature (280)..(280): n is a, c, g, or t
ttcttgggatattgatgactactgtctgagaggtgctgtggggagattttcaggattgtgtggtctttgaggggggtgtttttttaagacaacattgaccactgtccactgtccacatgatcattgtaaaattgcaatgccgcatgctagttggttacataagacataattccagtgattgaaggtggttacactgtatggtggtgtgttcaagatggcactggcatctttgagcagagcctggctatgcagcatcatttgagttttttaaacaccctanaggtctggttgttgttgctgttgtcctttcctgtgaaagtcacaananaagttacagtccaggtgaacctggagtttataggttggttttgtttctgntatatatatatatatatatatttttttttttttttaacatttacctgtagtgctgtagctgttgatactatcacctgcatgctatttctagtgagtgctaaatacagtatggtccaatgacaataacagcccatggtactgccag

SEQ ID NO: 125 - DNA, 487 bp, Homo sapiens
  misc_feature (483)..(483): n is a, c, g, or t
catcagtctgttatccatgctgactttccgaagacttgcagctactgcattgatatctttcctgccaataagcaaagtgttgaacacttcacaaaatattttactgaggcaggcttgaaagagctttcagaatatgttcggaatcagcaaaccatcggagctcgtaaggagctccagaaagaacttcaagaacagatgtcccgtggtgatccatttaaggatataattttatatgtcaaggaggagatgaaaaaaaacaacatcccagagccagttgtcatcggaatagtctggtcaagtgtaatgagcactgtggaatggaacaaaaaagaggagcttgtagcagagcaagccatcaagcacttgaagcaatacagccctctacttgctgcctttactactcaaggtcagtctgagctgactctgttactgaagattagggagtattgctatgacaacattcatttcatgaaagccttccanaaaa

SEQ ID NO: 126 - DNA, 274 bp, Homo sapiens
  misc_feature (233)..(233): n is a, c, g, or t
cacactttcatgataaaaacagaacctaggaatgaaaagaaattatagcaacataataaagaccatatatgagaagcccacagctaacatactgtatggtgaaaaactgaaagctcttcctctaagatcaggaacaaggcaaggatgcccattcttgccacttctatcgaacgtagtactggaagccctagccagaacaactaggcaatagaaagaaattaaaggcatccatntcagaaaggaagaancaaaatgctgtctgtttaanatgaca

SEQ ID NO: 127 - DNA, 130 bp, Homo sapiens
  misc_feature (9)..(9): n is a, c, g, or t
tttctatanaaaaaaattttttaaaataattgtaaagttagatttaaaattgtaaaatataaaatcacaaaggaatgtacccaataaaatgtaaatgcnccataaaaaaaaaaaaaaaaaaaaaaaaaaa

SEQ ID NO: 128 - DNA, 519 bp, Homo sapiens
  misc_feature (3)..(3): n is a, c, g, or t
cgntaacgtgcaatccgccgcacgccagcaaactggacaaactccgggatctcatcgaagcgattgagcaccagtaccagagtaataccggactgatgtaacgaggcgagtcgctcatccagcttgctgacgtgaggcaacatccaggccatcgaacggntcatcaagaatcaacaagtcaggctccgacatcagcgcctgacacagcagggtttttcgcgtctcgccagtggaaaggtatttaaagcgtcngtcgaggagggcggtaataccgaactgctgcgccagttgcatgcaacgcggtgcatcctttacttcatcctgaatgatctcagccgtagtgcgtccggtgccatcttcgccagggccgagcatatcggtgttattccgctgccattcgtcgctgacgagtttttgcaattgctcgaaggagagacgagtgatgtgggaaaactggctttgccgttcacctttcaaaagcgggaagttcccccgccagcgcgcgggccagggcccgat

SEQ ID NO: 129 - DNA, 272 bp, Homo sapiens
  misc_feature (31)..(32): n is a, c, g, or t
ttttttttttttattctattaaaaaatgttnntgaaaaaagatacttaaattttaaagataactnaattcctaangatttaaaataatccaagcagagatgaaagancaaatgcaaatgcntaaaaagaccccanagcattgttagcaaaaagcaaatatagttagccaagcatatatatntcataaaagcaataanaaggcntaaagcaagtttggggagagcttatttaaaacttgtaaaaatcatttgaatttttaaaagttttcaaac

SEQ ID NO: 130 - DNA, 551 bp, Homo sapiens
  misc_feature (144)..(144): n is a, c, g, or t
tttggaacacaaagttccctttttagaagaataggtattgagcccttgagcgtgggtagaaagatagagacagagtgatttgcaaaataatggaggatcatatttatatatgaattttcacttatttgaactttcagatatcancttnaaaanctttggtttaagtaaagtntnttaatgagactccttggatgaaagtaaccaaaaccagtaaaaataaggtaataaggatgtaatagtttcttatggacactcaacagctagaatgcagttagtctcagaaaagaattagaacaaataactggaaggccatcaggagtccaaaaccatcactcttttatattttatattttatttttctctcttcanatgagcattctctttctatgtccatatggtanaaggcggcagctccatagattatggcttcagatgttacagttccgctnaatgcagggacagacttgctatctttcagtccccttacatatcctggggagagagcaaatgattgactggcttgagtcaggtgcccgttccctttccaatct

SEQ ID NO: 131 - DNA, 224 bp, Homo sapiens
  misc_feature (6)..(6): n is a, c, g, or t
gtttgnttgtgaccatctgtacttgtaatttctttacnttcattggtatgaaaaatatgttcttagaagcangaaaaagaattcagntttgctttgtatactaaattaaatgctgtaattttgataaaatgaaaaatctgctttatttgcaacaattggtttcttccttgacgtcagcctcactcttggactttggtattcagccngncacccctgggaattcc

SEQ ID NO: 132 - DNA, 349 bp, Homo sapiens
  misc_feature (325)..(325): n is a, c, g, or t
gtgcctccctgtgtgagtagcctaaggtgcattgaaaaagactgggatgtgttttatttttttgtattagatagcattaaccttactgttgaagtatttttggtggagtattagtgacaagccattgagtcttaagccttacggcttcctataaaatcactaatttcgtgtgtgtttgtgtgtaggttacgttatatataggattcgtgttcgccgtggtggccgaaaacgcccagttcctaagggtgcaacttacggcaagcctgtccatcatggtgttaaccagctaaagtttgctcgaagccttcagtccgttgcagaggancgagctggacnccctggggggctc

SEQ ID NO: 133 - DNA, 635 bp, Homo sapiens
  misc_feature (557)..(557): n is a, c, g, or t
ttaacagctgcatagagttttaaaagtacattatattttgtcagacaagtaaaatatctgtttttcacgcaaaaaaagccatgaaatacgtaattttttaaagacaaaaaatcatcttttgagtttgctctttggtttttcttcattccttttgaggattgggaaaacagaaagattctttgatttgggtaatgaagaggtaatttgggacagtgtggtggtaccaggaagaaagaggattggaaaggccagtactgttttagttgctcggcactgttggttttgttttaatgtggttgccctgtccactacatggttctatcagtagtgtaatccattttcaatgtaaagctcttttagtttttgtcatagacataaattaatattttgagaggcatccctcacctgttcatttcttctgtgttgaaatgaagtacttaaaattaccgttatacatgaactttgtggactgtaagatttgttatatatgttcaaatgccttttagctggctttttaattaatatgcctgttttgagtgcttaatacaatgtaatgnggattgtaaatcatacctattttaaatcattccttcctgtatatttgnactcagagagccttattttattcttccagc

SEQ ID NO: 134 - DNA, 382 bp, Homo sapiens
  misc_feature (193)..(193): n is a, c, g, or t
ttttcttagaactttattttttctggccaggcgcagtggctcacacctgtaatcccagcactttgggaggccaaggcaggtcgatcacctgaggtcaggagctcaagaccagcctggccaacatggtgaaaccctgtctctactaaaaatacaaaaattagctgggcgtggtggcgcatgcctgtaatcccanctactcaggaggctgaggcaggagaattgtttgaacccgggaggcggaggttgcantgagccgagattgcgccactgcactccagcctgggcaacagagcgaaactccatctcaaaaaaaaaaaaaaaaaacaacctttattttttctgattttaaaagtaataactagtttgtagaaacattaaaagt

SEQ ID NO: 135 - DNA, 503 bp, Homo sapiens
  misc_feature (27)..(27): n is a, c, g, or t
accctaaacataacttaaaatttgttnggaatttgaaagtacagaattttcctgtaattgagactntttaaacttttgtggttggagaaggtattctattttttgaaaatatctgtaagttttatctaaatagtaaactctaagtattcttcccctttacttacagccaccctgggaatctgagactagagaaaataaagtttgtctcttgttctaaggagggtctggtttagaaatctgatttagacatagaaaaattgcaagaagcttgaggtgattggaagatacgattttgttatcaaagnatgtttctgttttatagattttattcatctacaactccttattaatatatttaagaagtcattaacccaccattgattacttgatataaaaggagaancggtggtaaaaggtgaaatanaatttttaatttttttttttttaagtttaggatttttttttaaattctaagagtttctgtcatttggggacaatcagaa

SEQ ID NO: 136 - DNA, 576 bp, Homo sapiens
  misc_feature (15)..(15): n is a, c, g, or t
tgggaatcataattngttaactgaagctnataagatgagagcattcanagagaaaagaacggaaagattgaatatcagtttcccttctttaaaaaaattgtggatatgtgatctagcttcttgagcatcacagtgactgattggctcgtggtaattgatcgctatgctgacaatcttatctccacctatgtcattcaattttctaagaggcaaaatccttaatcaggaggagagtttagctctagctaaatttcccttgtccagcatgctcctgctcccccaacttgtggaaacagctaaaggattggactaggagcanaagtttggaatggttaaaatgtagcaacatgtgtttcctgaaacaaaattccactataataaaaaaagcatttgaatgctcccttgtaattctgttggagcttgttgccttttttatgacacaaccataatcagtgatagacagtagcataaagaagcaagagcaaagcaattaagtaataatagcactacaaaaatgtgtgctgtacttaccaaacacgacatttatgaattattanataggaataaggggatggt

SEQ ID NO: 137 - DNA, 508 bp, Homo sapiens
gacccagccatctaaataagttrtacatgttgcgtatttttttgttagggacttatcttccgaagaggaaaggtttatgaaacctaaagtaacaatgatagcttggaatcaaaatgatagcattgttggcacagctgtgaatgatcatgtcctcaaagtgtggaattcttacactggacaactgcttcataacttaatgggacatgctgatgaagtatttgttctggagacacatccctttgattccagaattatgttatctgcaggacatgatggcagcatatttatatgggatattacaaaaggtaccaagatgaaacattattttaatatggtaagtgaagtgagatgtaccttgatacatgcttgataatttgtttagagtatttgggttatgcggcttacccagaaattgatctgcttgttttggcagtttgtttttacaaatcaacatattcaaagcctgctaaatattagacagctacatgtatatacgtacatacatgaa

SEQ ID NO: 138 - DNA, 4 bp, Homo sapiens
tttc

SEQ ID NO: 139 - DNA, 431 bp, Homo sapiens
  misc_feature (384)..(384): n is a, c, g, or t
ctcgctggcgggaggccacgggctttccacagcgcgggggaacgggaggctgcaggatggtcaagctgacggcggagctgatcgagcaggcggcgcagtacaccaacgcggtgcgcgaccgggagctggacctccgggggtgatctggaccctctggcatctctcaaatcgctgacttacctaagtatcctaagaaatccggtaaccaataagaagcattacagattgtatgtgatttataaagttccgcaagtcatagtactggatttccagaaagtgaaactaaaattttaatccaggtgctggtttgccaactgacaaaaagaaaggtgggccatctccaggggatgtaaaagcaatcaagaatgccatagcaaatgcttnaactctggctgaagtgganaggctgaangggttgctgcagtctggtc

SEQ ID NO: 140 - DNA, 606 bp, Homo sapiens
  misc_feature (177)..(177): n is a, c, g, or t
gaagacctcacatctgagagctcatctgcgttggcattctggagaacgcccttttgtttgtaactggatgtactgtggtaaaagatttactcgaagtgatgaattacagaggcacagaagaacacatacaggtgagaagaaatttgtttgtccagaatgttcaaaacgctttatganaagtgaccaccttgccaaacatattaaaacacaccagaataaaaaaggtattcactctancagtacagtgctggcatctgtggaagctgcgcgagatgatactttgattactgcaggaggaacaacgcttatccttgcaaatattcaacaaggttctgtttcagggataggaactgttaatacttccgccaccagcaatcaagatatccttaccaacactgaaatacctttacagcttgtcacagtttctggaaatgagacaatgggagtaaatattacacaaatacttattcattgnggttatttttatacagtagtgagaagaatattgttcctaagttcttagatatctttttttggatgtgcaaaaatttttggattgacagtaacttgggtatacatgacactgaaatgccttactttggatga

SEQ ID NO: 141 - DNA, 564 bp, Homo sapiens
  misc_feature (62)..(62): n is a, c, g, or t
tgcctgcgggccaggacctcgcccagcccatgttcatccagtcagccaaccagccctccgangggcaggccccccaggtgaccggcgactgagggcctgagctggcaaggccaaggacacccaacacaatttttgccatacagccccaggcaatgggcacagccttcctccccanaggacccggccgacctcagcgcctcctgcaggctaggacactggtgcactacaccccatgcctgggggccgagattctccagcagaaagatgcaatattttttgtttcctttttttccatttttttctctaaggaatcaatatttcaatatgttgagtgtgtgtccaatgctatgaaattaaaatattaaataacatatttatggcattttcttgaagagtgtggttgaagaaatatttctccttttgtttttcttttttttttgnttgntactgccacttctttttaggagcaaatctccccaggggtgtacggnatttcttgactctgggaacagctgctacccccaagacttgccacgttgttctgccctcaaatggaattaagtg

SEQ ID NO: 142 - DNA, 390 bp, Homo sapiens
  misc_feature (179)..(179): n is a, c, g, or t
ggaatatggtcaggatcttctccatactgtcttcaagaatggcaaggtgacaaaaagctattcatttgatgaaataagaaaaaatgcacagctgaatattgaactggaagcagcacatcattaggctttatgactgggtgtgtgttgtgtgtatgtaatacataatgtttattgtacanatgtgtggggtttgtgttttatgatacattacagccaaattatttgttggttnatggacatactgccctttcatttttttcttttccagtgtttaggtgatctcaaattaagaaatgcatttaaccatgtaaaanatgantgctaaagtcagctttttagggccctttgccaataggtantcattcaatctggtattgatcttttcacaaa

SEQ ID NO: 143 - DNA, 546 bp, Homo sapiens
  misc_feature (87)..(87): n is a, c, g, or t
acccgccatcttccagtaattcgccaaaatgacgaacacaaagggaaagaggagaggcacccgatatatgttctctaggccttttanaaaacatggagttgttcctttggccacatatatgcgaatctataagaaaggtgatattgtagacatcaagggaatgggtactgttcaaaaaggaatgccccacaagtgttaccatggcaaaactggaagagtctacaatgttacccagcatgctgttggcattgttgtaaacaaacaagttaagggcaagattcttgccaagagaattaatgtgcgtattgagcacattaagcactctaagagccgagatagcttcctgaaacgtgtgaaggaaaatgatcagaaaaagaaagaagccaaagagaaaggtacctgggttcaactaaagcgccagcctgctccacccagagaagcacactttgtgagaaccaatgggaaggagcctgagctgctggaacctattccctatgaattcatggcataataggtgttaaaaaaaaaaaataaaggacctctggg

SEQ ID NO: 144 - DNA, 109 bp, Homo sapiens
  misc_feature (12)..(12): n is a, c, g, or t
acattttccggnccttttgccatacacagttacagagatcagtcaaatccataccaccactgagatctcatttattgccacagatgcacaaaataaataacccaaaatc

SEQ ID NO: 145 - DNA, 374 bp, Homo sapiens
  misc_feature (272)..(272): n is a, c, g, or t
ccagcaacgacccatacctcagacccgacggcccggagcggagcgcgccctgccctggcgcagccagagccgccgggtgcccgctgcagtttcttgggacataggagcgcaaagaagctacagcctggacttaccaccactaaactgcgagagaagctaaacgtgtttattttcccttaaattatttttgtaatggtagctttttctacatcttactcctgttgatgcagctaaggtacatttgtaaaaagaaaaaaaaccagacttttcanacaaaccctttgtattgtanataagaggaaaagactgagcatgctcacttttttatattaatttttacagtatttgtaagaataaagcancatttgaaatcg

SEQ ID NO: 146 - DNA, 357 bp, Homo sapiens
gtacaggaggtaaattggataccccatctaaggggatctgtgagaccaggtagttatttggaatgaaagagtaagatattaaaccagccagcatgtcaacaggtgggtgatagtcttgttctcacagacaacagatggccatcatcttaaaacaacatttatgttaaccagcagataagggactcctgcattgtcagtggactttgagcctgagtttttctacttgcataggtgaaagtggactgcaatgctagtataaatgccgtatgatgactagtaccccttagggagctccagtttgccttcctggggaaccacagaccccaagtgtaatttcctgaggacagcccgacttct

SEQ ID NO: 147 - DNA, 293 bp, Homo sapiens
  misc_feature (63)..(63): n is a, c, g, or t
gttactgtgagcctgtcagtagtgggtaccaatcttttgtgacatattgtcatgctgaggtgngacacctgctgcactcatctgatgtaaaaccatcccanagctggcgagaggatggagctgggtggaaactgctttgcactatcgtttgcttggtgtttgtttttaacgcacaacttgcttgtacagtaaactgtcttctgtactatttaactgtaaaatggaattttgactgatttgttacaataatataactctgagatgtgtgaaaaaaaaaaaaaaaaaaaaaaaaa

SEQ ID NO: 148 - DNA, 521 bp, Homo sapiens
ctgcggtggagccgccaccaaaatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaactgtatgagttaataaaagacatgaact

SEQ ID NO: 149 - DNA, 470 bp, Homo sapiens
aagctcatgattttaaatgtatttttctaataaactatactcccatttaaaaatcaccaataccttaatgtttcaattatataagctaattaaaaataaaggctgggcgtggtggctcactttggaagaccgaggcaggcagatcacctgaggtcaggagttcgagaccagcctgcccaacatggagaaaccccatctctactaaaaatacaaaattagccaggcatggtggcacatgcccgtaatcccagctactggggaagctgaggcaggagaatcacttgaacctgggaggcaggggctgcagtgagccgagatcatgccattgcactccagtctgggcaacaatagtggaactccatctcaaaaataataaaaaaaataaaataaaaataaaattcaaacctaaaatagatgctctacttcaggagtgggcaaattaatcacctgcatcctttttttgggctttc

SEQ ID NO: 150 - DNA, 575 bp, Homo sapiens
  misc_feature (16)..(16): n is a, c, g, or t
tttttttctaaatggngattactaatatatgtggagactattaatctcttttctgttgccattagttcatttttccccaaaagccaatacatgttcattacaaaaatgaattataaaatataagttaaaagaaaaacataaaaccctacaatcttacccacccagacaactactattaataccttagtattaacatatacacatcatgtatatgtataaatttatcttaaacaaaaataaaattattctttacatattgttttaaaacctatttatctggccaggtgccgtggctcacgcttgtaatcccagcactttgggaggctgaggcacgtggatcacctgaggtcaggaattcgagaccagcccagccaacatggtgaaaccctgtctctaatggtttaaataccaaaaaattagctgggcatggtggcacatgcctgtaatatcagctaacatgggaggctgaggcaggagaatcacttgaaccanggagggggaggttgcagtgagccgaaatcacaccacttcactgcagcctgggcaacaaagcaagactgtctcaaaaagaaaaa

SEQ ID NO: 151 - DNA, 470 bp, Homo sapiens
  misc_feature (53)..(53): n is a, c, g, or t
cactgtcattcccaggaggctttggagtcagaactggattcaaattctgactntatgttgtgtgacttgggccaatagcttctttntgtgcctcagtttctttagctgtaaatanacgggtaggtcaccccttaccccataggttatggggaaagttacagaaaatggtcagctgggcncagtggctcaagcctgtggtcccagcnccttgggaggccaaggtgagcagattgcttgagcccaggagtttgacaccagtntggcaacgtgacgaaaccctatcnctgtgaaaaatacaaaaaattagccaggcatggtggtgtgtgtctgtggttccagctgcttgagagtttgaagtgggaggatcacctgagcccagaaggtcgaggctgcagtgagctgtgatcgcgtcactgcactccagcctggcgacagagtgagaccccttttgaaaaaaaaaaaaaaaaaat

SEQ ID NO: 152 - DNA, 631 bp, Homo sapiens
  misc_feature (248)..(248): n is a, c, g, or t
gtgagcggtggtggtttattcttccgtggagttaagggctccgtggacatctcaggtcttcagggtcttccatctggaactatataaagttcagaaaacatgtctcgaagatatgactccaggaccactatattttctccagaaggtcgcttataccaagttgaatatgccatggaagctattggacatgcaggcacctgtttgggaattttagcaaatgatggtgttttgcttgcagcagagagacncaacatccacaagcttcttgatgaagtctttttttctgaaaaaatttataaactcaatgaggacatggcttgcagtgtggcaggcataacttctgatgctaatgttctgactaatgaactaaggctcattgctcaaaggtatttattacagtatcaggagccaataccttgtgagcagttggttacagcgctgtgtgatatcaaacaagcttatacacaatttggaggaaaacgtccctttggtgtttcattgctgtacattggctgggataagcactatggctttcagctctatcagagtgaccctagtggaaattcgggggatgggaaggccacatgcattggaaataatancgctgcagctgtgtcaatgttgaaacaag

SEQ ID NO: 153 - DNA, 646 bp, Homo sapiens
  misc_feature (200)..(200): n is a, c, g, or t
ttttttttttataaactccaatcatttccagagctacttagctcagcatcttttttttccacgctcttaagttgtgtttatacatttttgatacagttagattgtttttgtcacattcttcattctatcctgggatcccccaaccacctaagtggattttttgataatttgcatgctttaaggataactcttcattctgnaaagggctatgggttttggcaaatgcagagtcatgtatccaagattacaatatcgcacagaagagtttcatcactatataaaactcaccagtcttcctcctattcaaccatctccatgccttcttcccagccctaactccttaaaaccactcatatctttactattgctatagtattgcctcttccaccatgtcatataaatggaaacatacagtattagtcttctcaaactagtttcttttacctaacaacatgcatttaagattcatagtgtcttttaatgacttgatagattatttctttgtagctgaataatattgcatcttatagatgtaaccgtttgtatatccatattttctcacagcctatgacttgncttttgattctctgaacaggccattcacaaagcagaagttttaatttttataaagctaatgnatcaactt

SEQ ID NO: 154 - DNA, 69 bp, Homo sapiens
cctggatgacagcatatctgtttatagctcagtttactgaatactttaagcccactgttgaaacctgct

SEQ ID NO: 155 - DNA, 502 bp, Homo sapiens
  misc_feature (500)..(500): n is a, c, g, or t
gatgcatgtccagcataggcaggattgctcggtggtgagaaggttaggtccggctcagactgaataagaagagataaaatttgccttaaaacttacctggcagtggctttgctgcacggtctgaaaccacctgttcccaccctcttgaccgaaatttccttgtgacacagagaagggcaaaggtctgagcccagagttgacggagggagtatttcagggttcacttcaggggctcccaaagcgacaagatcgttagggagagaggcccagggtggggactgggaatttaaggagagctgggaacggatcccttaggttcaggaagcttctgtgcaagctgcgaggatggcttgggccgaagggttgctctgcccgccgcgctagctgtgagctgagcaaagccctgggctcacagcaccccaaaagcctgtggcttcagtcctgcgtctgcaccacacattcaaaaggatcgttttgttttgtttttaaagaaaggtganat

SEQ ID NO: 156 - DNA, 373 bp, Homo sapiens
  misc_feature (8)..(8): n is a, c, g, or t
ctgcgatngagttttgagaggaaggantaaagtnctcatctcngacggtgagaaagatcatnactaaggaaacgcagggttggaagcagtgctgantgtccagttgagtttcatgancaaacatttgctgtgggaccagttttcatggnggtttgtcattttgtccagctgcctggagctgcttggttgaaggcacagaataatcaggattaattgttnaacttgtatgaatttctttattttaaaataggaataatatctgccttgggagcaagttgtaagagttaactgaaagcttnaggaaaaactttcccttgctatttaagtagggctttacaagttacaattctatcacagttttaagattataaac

SEQ ID NO: 157 - DNA, 155 bp, Homo sapiens
  misc_feature (9)..(9): n is a, c, g, or t
gcggcgcanctgcggatccanaaggncataaacgancngaacctgcccaanncgtgtgatatcaccttctnagatccagacnacctcctcaacttcaagctggtcatctgtcctgatgagggcttcnacaagagtgggaagtttgtctcaaaaaa

SEQ ID NO: 158 - DNA, 585 bp, Homo sapiens
  misc_feature (559)..(559): n is a, c, g, or t
gatttactgtgggaatttgctcatgcaattatggaaacctagaagtcccataatatgccatcttcaagctggaatcccaggaaagcaggtggtgtaattctgagattgaagtcttgagaaccgggggagtcaatggtgtaactcccaatctagggcttaaggcccaaggaccagggctgctggtgtgcagatgcaaatcctggagttcaaaggattgagaaccaggagctctggtgtctgagggcagtagaagatggatgttccagctcaagaagggaaagtaagaatccgtccttcctccacttttttgttctattcagatgagccctcaatggactgaacgatgctcacccacactgtgagggctggtcttctttattcaatccactgacttaagtgctgatctcttctggaaacaccttcacagacacacccagaaataatgttctaccagccatgggcctgttacttagcccagtcaagttgacacagaaaattagctatcacaacatctgtgtgtgtatatacatatgtatttgcatgtgtgtgtatatatggngtatatatattcatgtgtgtgtatat

SEQ ID NO: 159 - DNA, 454 bp, Homo sapiens
  misc_feature (428)..(428): n is a, c, g, or t
cttttgccagtaggccccctgagtaggttcctctatcttttggcatgaccccagaagtctttgataacttccttgctttctgatgtgacaagacatccagggccagattgtccatatcctgccccggatgcacgatgcactgtttctccaagaatccctgtgtcctttgctgatgatgccatgattttaagttctctaatatagttttatctctttgtttcagataatgcttttgtgttctcacatgtcctgctctctctctctctctcattttggtgttgatcagtctttccataagattgtttatttcactagtccttcattcttcttttttctaaatttactcttcttgactagtatcctgtcacttctgaggactcatatttttgcaacttgaaaattattcttatttatttaagtatatgttnctgaaactctcattagacacattttg

SEQ ID NO: 160 - DNA, 346 bp, Homo sapiens
  misc_feature (191)..(191): n is a, c, g, or t
gttaaaaaaagtaaaaggaactcggcaaatcttaccccgcctgtttaccaaaaacatcacctggtagcatcaccagtattagaggcaccgcctgcccagtgacacatgtttaacggccgcggtaccctaaccgtgcaaaggtagcataatcacttgttccttaaatagggacctgtatgaatggctccacnagggttcanctgtctcttacttttaaccagtgaaattgacctgcccgtgaagaggcgggcataacacagctgaaaaaaaaaaaaaaaaaaaaaaaaaaaaaaaaaaaaaaaaaaaaaaaaaaaaaaaaaaaaaaaaaaaaaaaaaaaaaaatttt

SEQ ID NO: 161 - DNA, 516 bp, Homo sapiens
  misc_feature (43)..(43): n is a, c, g, or t
cttttcatggtctcttgttcattaatcatctaaaatccaagcncagagaattcaattttagatggtctccagagcagaatttgatgtataatcttaattacaaatcatagataattaatattgnttacaaaatcanaatacgattagaggtagggatcctgcacacaccctattttcctccccagtgttctgaccgagagactaattaataattcaaggaacttacagtgaatganaacccatggttttgcttaattatcagaacagctagatctgagaacagctgtctcccacatggatagacacttattccacccatttgcaggtagaatagctggcaataataagtccttcccattggatatgttgaaaggtgcctgccatggcatagttgccacaagagaggaagaaatggacacaaatgtaggctgttttcagggcanagggaaggtgggaggaaaccaanttgctggttttcacacaccctctggggaacacccatgcacctatganatg

SEQ ID NO: 162 - DNA, 385 bp, Homo sapiens
gacaaaagctgagagaatttttttcttgaatatttgcactaaaagataggttaaaattcttcaggctgaagagagcataccaggtggagatttggatctacaaaaaggaaggaagatttggaaatggatttggcaccattgactcaatttccagaacaagaaagcagggacagttttgggaagctcaagacacactgcccatgagcagcaatttggacctcctgctgcatccactgtgcatcaaacacacactgtacagacaaagactcccaggaaaagaagtataaacatggactaacacagagatgggcaaactacagcctgtgacccagccacctgtttatgtagaatccaaagtaagaatctttaacttacacataaactt

SEQ ID NO: 163 - DNA, 660 bp, Homo sapiens
  misc_feature (22)..(22): n is a, c, g, or t
gacagcagagcacacaagcttntaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaaganagctctgtctggaccccaaggaaaactgggtgcanagggttgtgganaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttagatataaggattttcctggatattgcacgggaga

SEQ ID NO: 164 - DNA, 549 bp, Homo sapiens
  misc_feature (22)..(22): n is a, c, g, or t
gacagcagagcacacaagcttntaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaaganagctctgtctggaccccaaggaaaactgggtgcanagggttgtgganaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgt

SEQ ID NO: 165 - DNA, 660 bp, Homo sapiens; identical to SEQ ID NO: 163
  misc_feature (22)..(22): n is a, c, g, or t

SEQ ID NO: 166 - DNA, 542 bp, Homo sapiens
  misc_feature (94)..(94): n is a, c, g, or t
gaattgtgatagttcagcttgaatgtctcttagagggtgggcttttgttgatgagggaggggaaactttttttttttctatagacttttttcanataacatcttctgagtcataaccagcctggcagtatgatggcctanatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtgggattgangangctgtgctgtgt

SEQ ID NO: 167 - DNA, 421 bp, Homo sapiens
  misc_feature (220)..(220): n is a, c, g, or t
cttgaacctcggaggcagaggttgcagtgagccgagatcacgccactgcactccagcctcggggacagagcaagactccatctcaaaacacacacacacacacacacacacacacacacacacacaaaacagatatacactgaacacagcacaagtgggacataagagatttaaaagggttagagatgtaaaatggatctaggaatggaaaccataaggngggatttatcaactggattctgcanaatgctgttaaggccagatgttagcaggtgttacataaaaaagggataccatgagcaaaagtatttgaacatgggcaatggttgaaacaagtttaaacagattatntttattaccaaatctctcaaacctttaatatgctataaacattgtgaaacaataaaaaaactttccaaaa

SEQ ID NO: 168 - DNA, 472 bp, Homo sapiens
  misc_feature (152)..(152): n is a, c, g, or t
gggaagggagctatgagtgtgtgtgttgtgtatggactcactcccaggttcacctggccacaggtgcacccttcccacaccctttacattccccagagccaagggagtttaagtttgcagttacaggccagttctccagctctccatcttanagagacaggtcaccttgcaggcctgcttgcaggaaatgaatccagcagccaactcgaatccccctagggctcaggcactgagggcctggggacagtggagcatatgggtgggagacagatggagggtaccctatttacaactgagtcagccaagccactgatgggaatatacagatttaggtgctaaaccgtttattttccacggatgagtcacaatctgaagaatcaaacttccatcctgaaaatctatatgtttcaaaaccacttgccatcctgttagattgccagttcctgggaccaggcctcanactgtgaaagta

SEQ ID NO: 169 - DNA, 638 bp, Homo sapiens
ggcggaggttgcagtgagctgagatggcgccattgctctcccagcctgggtgacaagagcaaaactccgtctcaaaaaaaaaaaaaaaaaaaaaagcaatttacttaaaaacatacaaacacagagacaagtatttttgagaaacaaatacctttttcattttttataccaatgtaacaataatccattaaacacacctttactaactgttttctaggagtctgatatgatgaggaaataggtaaacctttaatagccagtactaaattagagtggcacaactttcactgggaaaaaagatgggtattttacttttctgttttagaaaagtggcttgacaacagtatgcttatgtcttagagtttgaaattcaagttcttgaacattattaatggctacaatcattcatacccacattgggctgtattcttgatgaatccaaagtgattttcacctcaactctgaatttcattctcctcttttgaatataatacaaccatctcactagaggaagcatttcagtcttttctgattggagattcattattgttttagataatgttttcatttgcttatgggtatataaaaaattttatcttaaaaatatttcctctcatttagctagcaacattgttttc

SEQ ID NO: 170 - DNA, 512 bp, Homo sapiens
  misc_feature (432)..(432): n is a, c, g, or t
ctcagggtgatctctgaacccaaacttgccccaaagaaggttgctctgtcctctccacatccccatctcctccctagggccttgttggggagaggctcctccatctttcccaagtcacaccatcgtttcctacgtggtctggacaagagcaagagcacaccttgtccccaccttctccagagcagccagaacccacctcaggtgccttccccatccggtgcagttaaggcacttctgccagcaccatggtatgagcactagacttggagttaagatttgagagccccctctgtcactgtggaagcttgagcatgttgcttgatctctctgaaccttgtgtttctcatctgtgaaaggtgataatgtggggctgctgtgagatttaaaggacataatgcacctacggtccaagcactgcctggaatacagcanaagctcaacagatactggacaacccatccccttagtagaggcactaaccatgtgacccaaggcaaaagtgcttaaaaaaa

SEQ ID NO: 171 - DNA, 580 bp, Homo sapiens
attgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcactggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgag

SEQ ID NO: 172 - DNA, 541 bp, Homo sapiens
  misc_feature (188)..(188): n is a, c, g, or t
gcaacctgcacaaccccgccctgttcgagggccggagccctgccgtgtgggagctggccgaggagtatctggacatcgtgcgggagcacccctgccccctgtcctacgtccgggcccacctcttcaagctgtggcaccacacgctgcaggtgcaccaggagctgcgagaggagctggccaaggtgaanaccctggagggcatcgctgctgtgagccaggagctgaagctgcggtgtcaggaggagatatccaggcaggagggagcgaagcccaccggcgacttgcccttccactggatctgccagccctacatccggccggggcccagggaggggagcaaggagaaggcaggtgcgcgcagcaagcgggccctggaggaagaggagggtggcacggaggtcctgtccaagaacaagcaaaagaagcagctgaggaacccccacaagaccttcgacccctctctgaaccaaaatatgcaaagtgtgaccagtgtggaaacccaaagggcaacagatgtgtgttcagcctgtgccgcggnttg

SEQ ID NO: 173 - DNA, 671 bp, Homo sapiens
  misc_feature (610)..(610): n is a, c, g, or t
ggaatagaattttaaatagtaataactgcttgttttttttgtgcaagtacttttatacataagataaacaaaaaccttaccaccaaacataccaaaatgcacctctttcataagtgagttactaagatttctatacctggaatatcatgtatgtttcatttactggatgtttacattttaggaaggaaaatagttttgtttatttaaacaactgaatacttataaactgttgttcctggaagttatttattccataaaaaatttgttcttttgtcatgaatttataattcctaaatgaagaccagaaagtacaaattgctgggaggaagaataggctttattaatcaactgatgtcttgatttttctaaatgggaagattgctttatttttaacactaattatgggagcagattcttagcaaacttctttggaaaagttaatgttatgatgtgcattaggctgccccatcgtgtatataaatgaagcagatttgatttttgtattcttacgtttctctgctttgtagttgtggctgtacttaaagaaatacagaatttcatatatttaaaaatgtttaaaatgtgacccacagacattgtaaatggattnaaaactaacatgaaaaatattcaacctaaaagaattcttaacttcacaagtgttttacttc

SEQ ID NO: 174 - DNA, 607 bp, Homo sapiens
cttggttccgcgttccctgcacaaaatgcccggcgaagccacagaaaccgtccctgctacagagcaggagttgccgcagccccaggctgagacagggtctggaacagaatctgacagtgatgaatcagtaccagagcttgaagaacaggattccacccaggcaaccacacaacaagcccagctggcggcagcagctgaaatcgatgaagaaccagtcagtaaagcaaaacagagtcggagtgaaaagaaggcacggaaggctatgtccaaactgggtcttcggcaggttacaggagttactagagtcactatccggaaatctaagaatatcctctttgtcatcacaaaaccagatgtctacaagagccctgcttcagatacttacatagtttttggggaagccaagatcgaagatttatcccagcaagcacaactagcagctgctgagaaattcaaagttcaaggtgaagctgtctcaaacattcaagaaaacacacagactccaactgtacaagaggagagtgaagaggaagaggtcgatgaaacaggtgtagaagttaaggacatagaatttggtcattgtcacaaagcaaatgtgtcgagagca

SEQ ID NO: 175 - DNA, 583 bp, Homo sapiens
  misc_feature (94)..(94): n is a, c, g, or t
gtcaccaagagcttgttgtcaggttttcacttgctattcgcagagattttttttaaaggcactatttgtagtgttaaaagggtgaatttatcanaaggcataataatcataaatgtgtatatgcctaataatagaactttaaaaggcatgaagcaacactcaaaaggattaaagggagatcatctcacccccttcttaccaattgatagaatgatctgatgaaaacagtaaaataacaacagatctgaacactgtcaaccatcttgacaaatacttatgcctagtgttccattattggaacactaaacatgtggaatgatttatatcctactgctcaaggtcatcaccaaggtctaattgtaaaatttcaaaaaattgcaacctcaggcataaatgggttaatcgacatttatagcacacacatgcaacatgtaccagagattccttcttttctatgaacatggtacttccaccaagatagaccacattgtgaactataaaacaaatctaaaaacatttgaaatgaaggaaattatataaaatatgttctcttgatctcaatgaaattaaattaatactatat

SEQ ID NO: 176 - DNA, 549 bp, Homo sapiens
  misc_feature (417)..(417): n is a, c, g, or t
ctcatggcggccaatgtaggcccaaaacttcctcaagtcaaactctccaggcccaccttctgcttcccggtggcatcaacaggcccagctttgacttgagaacagcctctgcaggccctgctcttgcctcccaggggctttttccaggcccagctcttgcctcatggcagctgccccaggccaaatttctgcctgcctgccagcagcctcaacaggcacagctcctccctcacagtggcccatttaggcccaactcatgactgtgaggccatttccaggcctagtgcctgcctcgtggctgactcttgaagcccaaaacttcctcaaatcagccttttgcccaacttctgtctactgtcggactctacaggtcagcctctgcctcacagtggaccctccagacccagatggtgtctnctgtggcatcctcaggcgaagctcctgcctttcggcagcctctccaggcccagctcctcctgctccagccttctctccaggctctgaactttctcaggtctccctctgttgtccaaggctggagtgtagtag

SEQ ID NO: 177 - DNA, 662 bp, Homo sapiens
  misc_feature (581)..(581): n is a, c, g, or t
tatatatgtaatgcccttaacctagtgtttggcatgatcgttgctgaaagggaagcttgtgggtacagtgtcccctcagaagccaaagcccagggaaggtcgcctgcccaggtcaggctcccagcgagtttgtctggggaggggccattcatacctccaggtcaggacagaggctcgggctgagggaaccctacacaggtcctggaagcagatccttcctgcctaagccagcaggacagctcaacaggaagcatcttccagccacgggaggagaggcagcaccttttttggaaccatacagagctaagaatggtggtacaagtaatagattctgtactggcaaccccacttggtggagcaagttctaggaaaagggggctgtccttgagtcagccatggggtcagccacacagtcaccgcagctgctctttggcaccgggcgctggaaagacctaggatgacacagcctggaaagagcttgggaaaagctcatcttccacagaactacctgctataccagccagggcaggtgcttattcccacaacagccctctgttgtaggcggcagtgccatcctgaangtgccgtggtaccttctgaanacccagctgagggcctgtaatggcacttgcatgccacatggnacaccctttcccggttaa

SEQ ID NO: 178 - DNA, 339 bp, Homo sapiens
  misc_feature (14)..(14): n is a, c, g, or t
accgcggccgcgtnaanaaaaaaaaaaaaagaattccacttgatcaacttaattccttntctttatcttccctccctcacttcccttttctcccaccctcttttccaagctgtttcgctttgcaatatattactggtaatgagttgcaggataatgcagtcataacttgttttctcctaagtatttgagttcaaaactcctgtatctaaagaaatacggttggggtcattaataaagaaaatctttctatcttaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 179 - DNA, 457 bp, Homo sapiens
ttagagaggtgaggatctggtatttcctggactaaattccccttggggaagacgaagggatgctgcagttccaaaagagaaggactcttccagagtcatctacctgagtcccaaagctccctgtcctgaaagccacagacaatatggtcccaaatgactgactgcaccttctgtgcctcagccgttcttgacatcaagaatcttctgttccacatccacacagccaatacaattagtcaaaccactgttattaacagatgtagcaacatgagaaacgcttatgttacaggttacatgagagcaatcatgtaagtctatatgacttcagaaatgttaaaatagactaacctctaacaacaaattaaaagtgattgtttcaaggtgatgcaattattgatgacctattttatttttctataatgatcatatattacctttgtaataaaacatttttccc

SEQ ID NO: 180 - DNA, 658 bp, Homo sapiens
  misc_feature (11)..(11): n is a, c, g, or t
cgtctatttgngtttcttctcacaattggtaagttctctgtattgattgatggctaagtttgattagtgtttttctctagttggtaattatattctagtattttatcatcttattgtttactcaactnaaagtgncacagaagagttgccaggtttctctttgatatgagatctctnnttgatttggaatgcaaatcanaagtgtcatgttttgaataaagggaccagatgacttataggtattctttctctaaatataactaaggtaagatttttgttttgaggtacttaatctatataagtggtaaagaatttacttgaatttctccaaattctcatgtctaaagtctgattgattaaattcattcttggtatttcattttgaaaagaatgtagctttagcaaacctctttgtataaatgcagtgggattaaggtcatttaaaaaattgttatatcattgtatttttaaaatttaccagttttatttttctttttaccctttagcccggcctcagaaagtgtgtttgtgtccatttctcccagcgcaccctctgcatatctctacccacttgtcataattcagcatccagcagaggaaaacaaagtgttgcgtacagttcctctactagcagcatgcctcccccaggacaagtgta

SEQ ID NO: 181 - DNA, 452 bp, Homo sapiens
  misc_feature (217)..(217): n is a, c, g, or t
ttattgctgacataaaaatggtgcacatcggccagggcccaggatgaatcagccaatctgcaccatttatacatggaactggagaacattgtgccaataatcatttaatatatgccaaatcttacacgtctactctaaactgctctaatgaagtttcagtgaccttgagggctaaagattgttcttctgggtaagagctcttgggctggtttttcanagcagagttcttgttgtgggtagactgtgactaggttcacagcctttgtggaacattccgtataacggcattgtggaagcaataactagttcctatgaaagaaccagagctgggaagatggctgggaagccaggccaaagtgggggcaacagcttgcttctctttctcttctcaccctcagtttgtatgggaaaatggagatgtcctctccactttatcccacgatatctaaatg

SEQ ID NO: 182 - DNA, 209 bp, Homo sapiens
caggatatcgagaccatcccagacagcatggtgaaactccgtctctactggaatacaaaaagttagccgtgtgtggtggcacgcgcctctaatcccagctattcgggaggcttaggcaggagaattacttgaacccgggaggcgaaggttgcagtgagctgagatcgcaccattgcactccaccctggcgacagagcaagactccgtct

SEQ ID NO: 183 - DNA, 541 bp, Homo sapiens
cagccaacccagaaggagccagtctacaactatgcctgatcctcctcatggcaggccacgaagcattgctgccatgtgttgaattataaaacccacattgctttttgaaccctgttgcgggtaaaaataaccaaattatcagtccttggaaacccaggcaatcaagtgagtacaaggtaaagataagtatggtttagaggagaaattatgttcctgaactggtgtcctttgatggcagcgtcagccttgctaagtcagagtagagggagcagtgaccttaataagctttggtgagcatcatgtgcacgcgtgggtgggagtccctttcactgatgcttttaaaagtgcttttgcagaccctggaagggatcctccacacatatgaggtgtgggacaggtaggccagagaggattagccctgctttcgagactagaaatctacagtcctgaaggagcagtaattaattggtacacctgtcagggccagcccccaggtctcctggctttttccaggttttctgtctcacatgattttgctttt

SEQ ID NO: 184 - DNA, 640 bp, Homo sapiens
ctttaatttttcaagtgtttaaaaaacaattttatacttaagccagccttgaagataagcacaaaatttaccagtttacatttaaaaaacaaacaaaaaacgacaacaactcaagcacccgctctgtgcatagcactattctaggtgcaataaaagggaatcttaaccttagaaatatgagttcactttctggaattgtattatctccttttccagagagtaaaaataaataaaatcaccattgtttactacagatctgccccaaaccacatctggttcacagaaaggctaatttctgccaaattaaagatgtaatgaactcagttcctgctttcccaaaaacacgaaagcagaattccttttcactgaaaaaaataaacagttttccatgcaagggcagtttgcttctaataagtattttttaaaaaatttttttttcctctagcttttctttaaattttcttcctctaatattgccttttcttgtacaaggcagaccaggtatctttttatgctgtttttcctttactaagaaaagtattgcatcttgaagacaaaccatttcccagagtagtgataaaaaataacactaaaaaaactttaaaggtgagtcacttcatcaccttgatgaagtaaaaaa

SEQ ID NO: 185 - DNA, 633 bp, Homo sapiens
ggaaaaaatatttccacttagatattttacatggttttgtttaaaattaccattacttgttttttaaaaacacatgaccacatatgtatatgtatatctacctaaacattgtatcatggtttcagtatgttattcatgtattactgggagatgctaccaagaaaccaacccaaagaaaattctggaaaatacatttctatttatagaataaatgtttcatttatataaaagcaaaagaacttagagttctaataaatgggatgtctaataaattatgaagttactgatttgaatatattatatttttataacttccttgccaaagtcctgatttagtacattagagaacctgtgtttcctctctcctctaccattcatctctcttccatacagtcatttgggctttttactcaaagagaatcaagaaataataaggtataacaagcttggcaaagtgttggctttttaaaaaaaaatttttttaatctctagcagtttggtaatttagcagcatcatttatttgggattcttttatctgatttcaacagtgaaaaacatccctatgataaagcctaatgacccatttccaaaagatggaattgcccttcctagaaaatatgacggagaaaagt

SEQ ID NO: 186 - DNA, 502 bp, Homo sapiens
  misc_feature (210)..(210): n is a, c, g, or t
cgaatagccaagtggtctgacaagatcgagagtaatgaggcccatactttagtacagtcttgaatggccagatggtgctgggcataccccaaccagagatatgtaagtctttatgttgtcaaaatttcccagaaacatgaatttcccactaagattcattaaggaaaactagaatgaaaacaaaaacgttccttgtataatattcattanaaagaaatgaagaaggccgggcatggtggctcacgcctgtaatcccagcactttgagaggccaaggtaggcagatcatgaggtcaggagtttgagaccagcctggccaacatagtgaaatcccgtctctaccaaaaatacaaaaaaattagccgggcatggtggcacacacctgtcatcccagctactcaggaggctgaggcaggagaattgcttgaacctgggaggtggaggttgcagtgagctgagattgcaccactgtactacagcctaggtgacagtgcaagactctg

SEQ ID NO: 187 - DNA, 316 bp, Homo sapiens
  misc_feature (55)..(55): n is a, c, g, or t
cctatgccaaactaaagaaagcttgcctggcctacaggcctaaaggttcaaatgnggattaaaaaaacacagtagtcacataaaatgtctgctggctggctggaattccatcacctacaatttacctgctttcaaaaactgtgttcaacattgagaaaacagaaaaccacttatcttgagcttaatatgggcttctttttccttaactgtagaacacttactgaaatatcaaatcaatggttaggatatgtatcctaggcaggcctaaaccattaacacttggtttaagcaactttgtataattnacctcctaaat

SEQ ID NO: 188 - DNA, 316 bp, Homo sapiens
cttcatgagtgcccggttgcccaagtcaaaaacctgggagtgatataaactccccacacatccagtcagtcactcatcaactctattgattctgctgctaaatatatctcaattgtattaacttaaacatatgcataatacatcttcttcttcactgcatttttgtgggctgcacttacctttcaggtaacaacaacactggcccctcttgcccttctagtcagaagtgccaaaatgatgagagctagccatgacaaacccacagccaacattacactgaatgtgcaaaactggaagggcatccaaacagaggagg

SEQ ID NO: 189 - DNA, 644 bp, Homo sapiens
tagaattctcgcctgccttggcttctccctctagttgttccttctctgtcttctgtgggcttcttattgtctgctcactccttcttcagtgtcctctcatgggcttccttcccttctcagctgatgccatcacctggggaatcacagttactcagcagcactggggcctctctatctctatgctggtcatgcctatgtgtgagctgcagacccagtggaatttccatttgtgcatcccatgcccagcccaccctccaccagcctcgaatgcagctgttcagccctaccccagtcctcagaaaagttcctctccctggatcctctttttccttcatgagtgcccggttgcccaagtcaaaaacctgggagtgatataaactccccacacatccagtcagtcactcatcaactctattgattctgtctgctaaatatatctcaattgtattaacttaaacatatgcataatacatcttcttcttcactgcatttttgtgggctgcacttacctttcaggtaacaacaacactggcccctcttgcccttctagtcagaagtgccaaaatgatgagagctagccatgacaaacccacagccaacattacactgaatgtgcaaaactggaagggcatccaaacagagga

SEQ ID NO: 190 - DNA, 631 bp, Homo sapiens
ctgaggtgggaggattccactctcacccatttcttctttcattttcagtttctccagttagtaactgaagatgttctttgagtaattaagtgagtgagaaaatttttaagtgagaaatctataaaaagaaccatgttaacataaatatttcagtccttacaagttggtattgacttttctcattggtaatctgactgatttaatactgctcattccaatatctggtgatgtaattctggttatgaatccttgtattaataacacctcctgggaggttttttttccccaacattacattcagaatattagagctgaaaataccttttttaaggttatcaggaggagggagcttatgtttaatgtggtggataaaacttaactgctggttaatacaattgttattcaggtgaaattccctaaacttttcacgtgcaaagttttgtatgtatacagacatttggggaaaagttttatcatccctaaaaccggttactgtccagaaaatgataagaatccctgggttccaaatccttcataaggtatttattcatttatttattcaacacatttactcaatgcctccgctctgctgcaactacactgacattctgcttctaatctaaccgaaaat

SEQ ID NO: 191 - DNA, 638 bp, Homo sapiens
tttcaaattgtacaataacacaaacaactttgttaaggccatgttttatttgctgattaatggacaaaaggcaatgtaatttattttcaagtattttcttgaaagtctgtgctcataaaaatcatgaaaagttggaaagactgttaaatcactgaaacttcaaatatatcttacacaatcttgtttgtacaaaaatacaagttaaatataaacataaagcaatcatggtaattttatgcaaatctgttttatgtgatcatcagttatatataaaagtttctcagttctgttatttgtgaaaagatcaataccagattgaatgactacctattggcaaagggccctaaaaagcttactttagcactcatcttttacatggttaaatgcatttcctaatttgagatcacctaaacactggaaaagaaaaaaaatgaaagggcagtatgtccataaaccaacaaataatttggctgtaatgtatcataaaacacaaaccccacacatctgtacaataaacattatgtattacatacacacaacacacacccagtcataaagcctaatgatgtgctgcttccagttcaatattcagctgtgcattttttcttatttcatcaaatgaatagctttttgtcacc

SEQ ID NO: 192 - DNA, 283 bp, Homo sapiens
gtaaactgttctctccgagggaaaaaatggaagttatcctcacagttcactgccgtggtatttcttctgtcccatgctttgcatgactgccatggtacagccttgtttcaaactgttcactgtgatctgtgggtctttgagtttcagtgagtttgctgaaatgtcgaagaagtagttccaaacttcaatgttcaatgaaatttttgttcaagtttgaaatggagagagcagctttaaaaggtactaagccttttacaaattggtgagtactggcacatgagat

SEQ ID NO: 193 - DNA, 613 bp, Homo sapiens
  misc_feature (68)..(68): n is a, c, g, or t
tttttttttttccttaaaaggtaacccctaaacacagctaaaactatgccatcagctgactccaaggnacacacagtcctgtatctggaactactgagtggcaggcatctttctctgcctctgacagtggagtccccatcactgcagagcatagccaaaggagtcaaaggtctcagcgggtcactgccttatcaaccctcaccagtcccttatgttttttaatattttataatcttgacatgacaccaagatgctttaataaaaaagcacctctaactcggtcttgtattcacttaccttgagcctgggacttctctaggctcctgaggcaaaaacaggtagaggggagatggtggaacataaaacacaattttgcttggcacccaccttggcgtctgtccccatgaccaggtctttcaattcgatgattttgtcattgatggaggagcgatatcgtttctcaatgatattatgggttgtccgcctttctccttctttggggggctcaagctgcttgactcccccaggtacctgcttaatggggcactttctcttgccccatcattacaggcattgtggtcagaatggtcccactgctgcccaccagggtcta

SEQ ID NO: 194 - DNA, 350 bp, Homo sapiens
  misc_feature (261)..(261): n is a, c, g, or t
ctagtcttttcatagtctgcatagagtctggccattaccatcagtttttaagatgtccatattgtggccgggcgcggtggctcacgcctggtagtcccagcactttgggaggctgaggcaggtggatcatgaggtcaggagatcgagaccatcctggctaacacggtgaaacccgtctctactaaaaaaaatattaaaaaattggccaggcctggtggtgggcgcctgtggtcccggctgcttgggaggctgaggcagganaatggtgtgaacccggaagtcggaggttgcagtgagccaagattgcacctgggcaacacagcgagactccgtctcaaaaaaaaaaaaaa

SEQ ID NO: 195 - DNA, 541 bp, Homo sapiens
caattatttattacctttccatttgttcgcctgatgatgtgacaatgcatggtctttgtgcatgctgctagacacttttctttcccagccgaaaagtctattatgtaatttttacattcataattttaatgtggatgatcaggattaaatcaagatatatatctggaacctcttataaatggagcacttagaaatttgttgttctgcacttaacctagagagagaaaaaatgcttttctttgtgaaaaatctgaattcctgtcctgaccttctgtgatgtggaaaccctaggctctgagacacactctctggtgtctgagacagaaccaaagcaataacgttgtgatgcccacaggcctggagccagctagcgaccttgtgccgcccagctgtccatggcccgtgcagagcagaggacagtgagtgtctgcactgagaaccttaaaccacagttgaacatacccacacctgtttgtcttaagctatagtgtaaaaacaaagtttgggctctgaaaatttaactgaaaaagatttccttgtt

SEQ ID NO: 196 - DNA, 336 bp, Homo sapiens
gtggcagcaggcgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatggccagtttaaaacttatgctatctgcggggccattcgtaggatgggtgagtcagatgattccattctccgattggccaaggccgatggcatcgtctcaaagaacttttgactggagagaatcacagatgtggaatatttgtcataaataaataatgaaaacctaaa

SEQ ID NO: 197 - DNA, 377 bp, Homo sapiens
  misc_feature (128)..(128): n is a, c, g, or t
cagcagcagaaatgtttgcaagataggccaaaatgagtacaaaaggtctgtcttccatcagacccagtgatgctgcgactcacacgcttcaattcaagacctgaccgctagtagggaggtttattcanatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgt

SEQ ID NO: 198 - DNA, 63 bp, Homo sapiens
tactcaatgaaaaaccatgataattctttgtatataaaataaacatttgaaaaaaaaaaaaaa

SEQ ID NO: 199 - DNA, 565 bp, Homo sapiens
caggatcaaggtgaaaaggagaaccccatgcgggaacttcgcatccgcaaactctgtctcaacatctgtgttggggagagtggagacagactgacgcgagcagccaaggtgttggagcagctcacagggcagacccctgtgttttccaaagctagatacactgtcagatcctttggcatccggagaaatgaaaagattgctgtccactgcacagttcgaggggccaaggcagaagaaatcttggagaagggtctaaaggtgcgggagtatgagttaagaaaaaacaacttctcagatactggaaactttggttttgggatccaggaacacatcgatctgggtatcaaatatgacccaagcattggtatctacggcctggacttctatgtggtgctgggtaggccaggtttcagcatcgcagacaagaagcgcaggacaggctgcattggggccaaacacagaatcagcaaagaggaggccatgcgctggttccagcagaagtatgatgggatcatccttcctggcaaataaattcccgtttctatccaaaagagcaataaaaagt

SEQ ID NO: 200 - DNA, 629 bp, Homo sapiens
cagaagagtaagcaaatctcaaagcagcgaaagggaagaaactaaaaaaggtagagcagaaataagagaaaatagagaagagaacaattgagaaaaataattgaaaccaaaaggtggttctttgaaaagcctaacaaaatggacacatctttagttagagtgaccaagaaaaaagggcagtgactcagattacttcattcaagagtgaaagagggcacatcactaccaatttacagaaataaaaaggattatgaggaaatactacagataattgatgacattaacttagaagaatatatttcaagaaagacacaaactactgaaaccgactcaagaagaaacagaaaatctgaacagacctataaaaaatagagatttaattgatattcagaaagtttcccaaaaagaaaagcactggccaagatgacttcactggtgaattctatcaagtgtcaaagatgaattactgacattcattcacactcctttaagaaatagaagaggggacatcacttttcaaagcatcgacattctaatcattagtcccttggtttcctgctcccaaagccaggtgatgtatcacaaaaaaacccctacagacccactgggcacaatggctttatgcctat

SEQ ID NO: 201 - DNA, 98 bp, Homo sapiens
  misc_feature (13)..(13): n is a, c, g, or t
ctttgctcgaatngtcagataaggattctgtgaanggagatgagatttccatccatgctgactttganaatacatgttcccgaattggggnccccaaa

SEQ ID NO: 202 - DNA, 224 bp, Homo sapiens
ctcaagtgttccctcagcttaggctttgtttaaatgatcccacccaggggcgatggtagggaacaacagggtcactaaactatttggctggctacaactctgggaaatggtaagacagggaaaggccatgttgttcattcccttgtgcagatctagggagaaccgcagagagaacagttagcatttcttgttcaatgaattatcctattaagaacactggatgt

SEQ ID NO: 203 - DNA, 81 bp, Homo sapiens
  misc_feature (4)..(4): n is a, c, g, or t
cggncgcggtcgacgctactcctacctatctccccttttatactaataatcttataaaaaaaaaaaaaanaaaaaaaaaaa

SEQ ID NO: 204 - DNA, 362 bp, Homo sapiens
  misc_feature (97)..(97): n is a, c, g, or t
ggcatgtgcctgtagtcctagttgctgaggtaagaggattgcttgagcccaagagttcaaggctgcaacaagctttgattgcgccactgcactccanccttggcgacagactaaaacgctgtctcaaaaaaaaaacaaaaacgacnaaaaaaaaacaaaacagaaaaaattaacttaggcaatgacagtccctggcaaatgctgggagggaggcaacantggtcaaggaaggtaaccctgaancaggacttgtaaagcaaataanattgggaggccaaggtgggtggatcacnaggtcaggagttcgagaccaacctggccaacatagtgaaaccccgtctttctaaaaatacaaaaaaatt

SEQ ID NO: 205 - DNA, 581 bp, Homo sapiens
gacaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctgtccggaatgccccgacgttactcggactaccccgatgcatacaccacatgaaacatcctatcatctggag

SEQ ID NO: 206 - DNA, 595 bp, Homo sapiens
  misc_feature (13)..(13): n is a, c, g, or t
ttcaaattcttgntaanagtctttgttctgaattttactttgtctgttattcctatagcctttccaattttctttcgcttggattttacgtgataagttttttcccccattttacttttancaactctatattttttagttgaggttgggtttcttgtaaacagcatataatttgggttttttaatccaatctgaaaattaatgtccttaattttgtgtttataccatttacacataatgtactcatatataaggtttaactgaaacctactatcttgctagttgtgctctacttgaatttttttttagtattctgttttaattgaccaacatttgactgtatctctttgtgtaattcttttacaggttgctgtaggcatgacaatatatacacttaacttttctcagtacactgagagttgaaattgtagtacttcgaggaaaacatagaaaacttgcaatgatatcggttacattttaccacctccatatgttgcaattattaaatgtattagatctgcctacctcgaaaacccatcagtcttttaactttgctctcaatggtgattcatatttttaaaaaaacttgaggcaa

SEQ ID NO: 207 - DNA, 522 bp, Homo sapiens
  misc_feature (504)..(504): n is a, c, g, or t
tcgaccgggtttggagcagtgccttgtttgctgtgcagcggatactctacaggtacatttcctttttggaaccaaaagggagggatttgacaatattgatggtagatcttttttctttagcaagaattaaggattttggtgggtggggggaggcttctgtggggaccaagacaatgtactgtcagtcaggatttaagtcgaactacctcatcccttgccccagagaacagttgatcgtgttttaaaccaaaaggtgcggaatggagagagggaggcggtgcattgcagcttccgatagagctttttatttttggatatcaggaaccaattttgaagatttcttaagaaagtcatttacatcagggacatgaagagcaaagtaggtatttttggtcagtacttgaatttgataggctttatgcaaacaactctccctctgctggagtctggcaagtttgcttttcactggacgctaattcaagtgccatacaaaactaaaataanagttttacttataacaca

SEQ ID NO: 208 - DNA, 585 bp, Homo sapiens
cagaaatcgcaattgaagaccagatttgtcaaggtttgaaactgacatttgatactaccttctcaccaaacacaggaaagaaaagtggtaaaatcaagtcttcttacaagagggagtgtataaaccttggttgtgatgttgactttgattttgctggacctgcaatccatggttcagctgtctttggttatgagggctggcttgctggctaccagatgacctttgacagtgccaaatcaaagctgacaaggaataactttgcagtgggctacaggactggggacttccagctacacactaatgtcaatgatgggacagaatttggaggatcaatttatcagaaagtttgtgaagatcttgacacttcagtaaaccttgcttggacatcaggtaccaactgcactcgttttggcattgcagctaaatatcagttggatcccactgcttccatttctgcaaaagtcaacaactctagcttaattggagtaggctatactcagactctgaggcctggtgtgaagcttacactctctgctctggtagatgggaagagcattaatgctggaggccacaaggttgggctcg

SEQ ID NO: 209 - DNA, 624 bp, Homo sapiens
gacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaa

SEQ ID NO: 210 - DNA, 338 bp, Homo sapiens
acctgaggcctcggtggggccagtgcgacgctggcttaaggagctggaggggttcctaatacacatttaattcagtttctcttccctaagaggctgccggagttggggcctcctccagcagagaccctcggacccctgcagggcctggacttggggtgaacagggcttcagtcagcgcaagtattccatttgcatttggtaatttttcatgccacctatttatgaatatataaatctttataccaaatctattttttaaaacatggaaaagttgcctttatggaaacttggcagagccagagtgtacacattcctaaaccattaaacagatttctata

SEQ ID NO: 211 - DNA, 556 bp, Homo sapiens
ggataatgatacctctgacctttcttccttttgggaagtacttgagtgtgcagctgcatgaggcctcagcaggagagagattttaggtccaagaagctataccagtaggacaaggcaggaaaatactacactttcaggatcaagcccctctgactctcatttggaaactggatgtttgctaagcacctgcttcttaaggatgccgagggatttaatgatactcccagaaacctggagagattaatggggcctatggagaagtgctctgaactcagtgttgggacttgaataaaattaaccattgtcatgttttcagaacaactaagctgttttatatttcatgtgcatgaaagccctagaactaagttgtgttatttccagaaatgaaatagatcccacagttagatgatgtggccattaggaagtaccaaatttataaaaatcactggaggtctgtctgagcagtacctaataaaatatagtatactgaaagtgaacagatctttgtctctttctttggctgcttgatactttatctgtgtctgccggacagtgc

SEQ ID NO: 212 - DNA, 305 bp, Homo sapiens
caataaaagcaggttaacctcaatgatagcagttaaaatgttctatcttatgtatttcttttaagtattaccattatggtgctactgagcgttttcttttggtaaaaagaaaaatgccatgggctgcagtcttcttccatcacttttccctaccaggtccattaatatgcttataacactagtgccagttattttatttgataatgcttatggtatttgtatatttgtttgcattccaattttgtttaataatgagtgtgtaaactgcatacgttaaataaatgtaaatactaatgtactgctgc

SEQ ID NO: 213 - DNA, 495 bp, Homo sapiens
  misc_feature (55)..(55): n is a, c, g, or t
ttttattacccaagttttaacctctgtctggtgatttgttgttgttgttgttgtngttgttgttgaagttcaggctgcatgtgggataggtttgctcaggcatacttcttaggaagtagtcacttgcatgactgtttttgggataactctttgagtatttggagaggtctattgtaacttctgaaaggcattgtttttacgtatgaatgttctaaaattcattctaaatggtcatgaaaagaaaaggattcacattttagaatggcaatagtccctgaggactattatgtcttttagatttcctgtgggtttctaggaatgttagtgtaacttanatttccacctacctgatttctggatgtgcctattggaacttgctgagatctttttttttccttaacatgttgtccccttgacccgtacttcgaaactaaacatattattttatttgcttacacttcaggaggcaattggcagacaccaggccaacagtct

SEQ ID NO: 214 - DNA, 507 bp, Homo sapiens
  misc_feature (437)..(437): n is a, c, g, or t
gctctgaccccagttggaaatgtatctgtactttgtccggcttccactcaaggaccatttatgacattgcttggtgtcagctgacaggggctctggccacagcttgtggggatgacgcgatccgcgtgtttcaggaggatcccaactcggatccacagcagcccaccttctccctgacagcccacttgcatcaggcccattcccaggatgtcaactgtgtggcctggaaccccaaggagccagggctactggcctcctgcagtgatgatggggaggtggccttctggaagtatcagcggcctgaaggcctctgagctacctcgactttggacagagtaatgactccccagaaaacgtcatataagactttaccagcccctgagaggaccaggaggagcatccttgaccttcatttaacttggctcacttctcttcanacttgggtagaagtgcagagccacaaaattgctttccttccccgcctttgacatgaggccttcagtaaag

SEQ ID NO: 215 - DNA, 17 bp, Homo sapiens
tgcaggatccgtcgact

SEQ ID NO: 216 - DNA, 576 bp, Homo sapiens
  misc_feature (566)..(566): n is a, c, g, or t
gagaaatataagattatgtatagatcaaatctacctctatttggtgtcctgaaagagatgaggagaatgggacaaacttggaaagcttatttcaagataacattcctgagaacttccccaatcttgctagagaggccaacattaaaattcagtaaatgctgaaaactccagtaagatatttcttaagaaaattattcccaagatatatactcatcaaattatctaaggtcaaatgaaggaaaaaattttataggcagctagagagaaatgtcaggtcacctacaaagagaatggcataagacaaaaagtagaactcccagcagaaactctaaaagccagaagagattaggggccaatatttaacattctgaaagaaattccaacaaggaatttcatatccagccaaactaagcttcataattgaaggagaaataagatattttccagacaagcaaatgctgatgaaatccatcaccaccagacctgccttataagagctcctgagggaagcactaaatattgaaagggaagaactttatgaaccatttcaaaaacacatttaagtncacaaagcag

SEQ ID NO: 217 - DNA, 341 bp, Homo sapiens
ccttattttacaggtgaaaaaccacgaatcagatagatttttatttgcccaagtcacataatattaagaacaggccaagtgtggtggctcatgtctgtaatctgagcactttgggaggctaaggcgggtggatttcctgagcctaggagtttgagatcagcctgggcaacatggcgaaacctcatctctacaaaacatacaaaaattagtcagtgtggtggtgagagcctgtagtcctggctactcgtgaggctgaggtgggagcatcacctgagcctgggaagtcgaggctgcagtggcaacagaatgggtaacctggacatcagagtgagaccctgtct

SEQ ID NO: 218 - DNA, 494 bp, Homo sapiens
  misc_feature (439)..(439): n is a, c, g, or t
ctcacacctgtaattccattactttggaaggctgagagaggaggatcagtggagcccaggagtttgagaccagcctgggcaatatagggagaccctgtctctacaaaaatgaaatagccaggcgaggtggcatgtgcctgtggtcccagctacttgggagactgaggtggaaggctgccttgagcccaggagttccaggctgcagtgagccatcattatgccactgcactccaacctgggagacagagtgagagagaccctgtctcaaacaaacaaacccaaaataggccaggcacagtgactcatgcctgtaatcccagcactttgggaggctgaaataggcggatcatttgaggtcaggagttcaaattcaagaccagcccggccaacatggcaaaaccacatctctactacaaataaaaaattagttgggtgtggnggagcattcctgtaatcacagctattcaggaggctgaggcatganaaccgcttca

SEQ ID NO: 219 - DNA, 379 bp, Homo sapiens
taaatttaaaacattttaattagctggcatgatggcatgcacctgtagtcctacctacttgggaggccaaggcaggaagattgcttgagcccaggagtttgagcttactgtgagctgtgatcacaccactgcactccagcctgggtgacaaaggaagaccgtatttctaaaaaataaaaaatacaaatacaactacaaactagcactagaccaacagtgactatgtaccatgaactgaggaatattattaattccaccatttgcatctgaggttaacaatatgtcaatgacttaaataacatcatatctctgagagtaatttctcctatatttccatgacaaatgttagataattttccattttttccattcaacaaaa

SEQ ID NO: 220 - DNA, 421 bp, Homo sapiens
ttttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccactcttactcatcacctgcagtggccagaattagggactcagaatcaaaccagtgtaaggcagtgctggctgccattgcctggtcacattgaaattggtggcttcatt

SEQ ID NO: 221 - DNA, 598 bp, Homo sapiens
gattaactttcattttaagctcttctctactaattctgttcgtatgtttattcattttgcgttgatcatattttgtacaccaggcactcttctcagttttatatgtgtgttaatttactcctttcaagagccctatgatacatgaatttatctccattttatagatgaggaaattaagacctagagttactgaacttgcccaaggttatacagctgatgggtagggccagaactttgcctcagagaatctgaatttccaaaaaataacctaaaagagaaatttaagtactaattagtaagcaaagaaatgcacatttaaggaagacagtgcacatttaaggaagacagtaaccttttatctattagagaaaaacacacattctgtctttaacacacacataaatcttatattggcagggattttctttattcagcaattatttattggttgtctgctttgtggtacacataaatgctggggataaacacttaataaaatatacttccttctcttgaatatcttgcactttaagtgggaaggtaagtcaacagagtagaggtgatatatccaagtgatagactgtttcattgccagtag

SEQ ID NO: 222 - DNA, 473 bp, Homo sapiens
gttgcctgagagtgacctttgcatctgcctgtccagccagcatggaaccaaagcggatcagagagggctaccttgtgaagaaggggagcgtgttcaatacgtggaaacccatgtgggttgtattgttagaagatggaattgaattctataagaagaaaagtgacaacagccccaaaggaatgatcccgctgaaagggagcactctgactagcccttgtcaagactttggcaaaaggatgtttgtgtttaagatcactatgaccaaacagcaggaccacttcttccaggcagccttcctggaggagagagatgcctgggttcgggatatcaataaggccattaaatgcattgaaggaggccagaaatttgccaggaaatctaccaggaggtccattcgactgccagaaaccattgacttaggtgccttatatttgtccatgaaagacactgaaaaaggaataaaagaactgaat

SEQ ID NO: 223 - DNA, 392 bp, Homo sapiens
tggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccct

SEQ ID NO: 224 - DNA, 618 bp, Homo sapiens
tttttcttgtttttgtgtgtctaccttggcatatactaaaggaaggtgtgtattcatttattacatgatatctctgggttataattatttacatatatgaatttgaaagaaagattgagagggatatgtgtgacctttgtttcattatgatcatttacatgactaaagataaagatcatatgtctgattttcagtttaatggcaagttacttaaaataaatgaaatatgtttttattgttttcgtgggtttgatgctttgtgttttatttcaagtaacttgagaatgcattgtgtttggtactgttttttatgaatatcattaaaaatttatttaaggagagagtaattttgcaataatatttttgatttatttgaaaataaaattcaagataaatgaaataattgaaattttctaaagaaggaattgaatatatttttacatttgaatgaactaaggattaactgaaccatttatatatagtactttcagaactgaatgtcttaaatgataaagctctaattggttaaagtgactttctttcaagtcaaagaacccagaaactgaatagatgatctaactactgccactgaggttttggattagtgagtataaattt

SEQ ID NO: 225 - DNA, 17 bp, Homo sapiens
tgcaggatccgtcgact

SEQ ID NO: 226 - DNA, 396 bp, Homo sapiens
gacaatcagagcagatcttgggcttctgtggctcatctcagccctttataactggcctgagaagagggtttatctacttgtgcaagtggcccagaaatctcactcgtacatgaggctttggaacatccttgcaaaggtacgctgaaagcaaattgctgttttcctggtggttctgcacgtttcctaacttttatcatagtttgattttcattatttaagaaaaaataaaaaatccaaagaccataagatggcattagattttttaccattaaattattaatgcctatttggtgctcataaagattaatcatgtcacgcatgtttccaatctttcttttgcagtatattattttctaaaaattgttacatgcaaatttaaaccaagatttatcagta

SEQ ID NO: 227 - DNA, 535 bp, Homo sapiens
  misc_feature (112)..(112): n is a, c, g, or t
ttggaagaaataaaccaaggcagaaaaattttaaatggccaaaataaattgtattgctaacttagatggccacagatgggggcaggggtggagagaggagaaattgaaaacnccacaaagaccccgcaatggctagaacttgaaatctctggatattgcaacaatagcagcctccttaagtcagcaaaaagataaagattgatccaatgttctatattacagaacagagcagattgtcaatatagcaaataaagttaccgttgagtggactgcgctgtntaagctgcttggttggccttaagtgccgacaattaagagatgaaggcaatgagaactgaaacaaacatttaagttcaagacccagtttactgacactgggactattactatatctctttgggcctcagtttacttatctgtaacattaagaggttggattacatgatgtctcacgattctttttttttatttagagatggggttttgctctgttgcccaggctggagtgcagtggcatgatcatagctcacagcag

SEQ ID NO: 228 - DNA, 392 bp, Homo sapiens
ccagcctgtcactggcctggccaaggaggagagacaggccagggattctggtcctaactctactggccacactgtgtggcctgagacccccctttccctcccaagcccctgcctccgcatctgcgtggtgaaggccattggccctcatcggtggatctgcgtttcctcgggcctacactgtctaggattgtgcggggctggtgagagaacaagatctcttccgtgttcaaggcagacttcctgccccctgcaccctgctctctcccaggccttgaggtcagtgtgagccccaagggcaagaacacttctggaagggagagtggatttggctgggccatctggatggaaggtaaaaaaaagaaaatcccttgaaaggagattgagggaagttt

SEQ ID NO: 229 - DNA, 419 bp, Homo sapiens
aagagaaaggactcagtgtgtgatccggtttctttttgctcgcccctgttttttgtagaatctcttcatgcttgacatacctaccagtattattcccgacgacacatatacatatgagaatataccttatttatttttgtgtaggtgtctgccttcacaaatgtcattgtctactcctagaagaaccaaatacctcaatttttgtttttgagtactgtactatcctgtaaatatatcttaagcaggtttgttttcagcactgatggaaaataccagtgttgggtttttttttagttgccaacagttgtatgtttgctgattatttatgacctgaaataatatatttcttcttctaagaagacattttgttacataaggatgacttttttatacaatgggaataaattatggcatttttt

SEQ ID NO: 230 - DNA, 622 bp, Homo sapiens
  misc_feature (551)..(551): n is a, c, g, or t
ctgagagtcactgtgtttttagccaaatctaagggagaaaatgaatattgatagcagcatgctgtagccagctccttaaaggaaggatggtgcctggtacagagttagagttagtgcttcagtaaataatgaatgtgtgctaggtaggttctgctgggtaggctgcatgcattgaccaatttattcctccttgtttcaaaacaggatttaagggcacttatatatatatattttttagtttttttaatgtaaatgagagaataaagatatatatatatgtctatatatgtatatatgtatatatatgtctatatgtctatatgtatatatgtctatatgtatatatgtgtgtgtgtatatatatatatatatatataagttttctgttgctagcataacaaactaccagaaacttagcaactgaaacaacatgaatttatcttacggttctatagttcagaagtctaacgtgtcactgggatgaaatccaggtttcaacaggactgggttcccttctagctcattcagctacctggctcattcaggttgtnggcagaatatacttccatgaaactgtagggctgagaccccgttccttcctggctatcatctgaaaactttc

SEQ ID NO: 231 - DNA, 350 bp, Homo sapiens
aggcgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatggccagtttaaaacttatgctatctgcggggccattcgtaggatgggtgagtcagatgattccattctccgattggccaaggccgatggcatcgtctcaaagaacttttgactggagagaatcacagatgtggaatatttgtcataaataaataatgaaaacctaaaaaaaaaaaaaaaaaaaaaaaaa

SEQ ID NO: 232 - DNA, 493 bp, Homo sapiens
  misc_feature (2)..(2): n is a, c, g, or t
tncactcacacactcccaaaccttaacaaacacatacatgtgcagccaacccaatgggccagcctcttttatgctcctcacatgtttcctttaactggaatacccatgacagctccctacatagttacttgtaaactcctcctctctgtataagttttcctgaatttttttgataaaattaagttgtgccacccctttatgctctcttanaactttgttctgttctcatggctgttctgcaacgaatctcattgtgttctcctactcaattacattcctgcgtctcccactagatggcagactctttgagagtaggagattcccttgttatctctggatccctggcacttgcagaaagcctgttacgtaataattgctcaacaattagtttttaaataaatgaattatttttaaaacgccaaaattacaatgattgtgcattaagtgaaagatgaccatctaaaaacataaagccatgcttcatgacattggc

SEQ ID NO: 233 - DNA, 577 bp, Homo sapiens
gaccattcagggaaattttataaaaaatgcagatactgtcttgagcagatcgaaatgccgatgaggtggatgcaatttccttttgtgcaagcagtgcacggtgcccccccctcgggtgtccgtgctgtgccttagcttccccaggtgccgggactcacacctgctaggggctgggcaaggccccggctctgctttctctgaagggcttgtccaagttcattgccctgttacaggtggtcaagacgtccggccgccttgacccaggctacccttagccaatatcctctgcccctgggtggttggtggctgggcctcagggtgggcaacgttaggggtttggcgaaagcccgccccatgggattgagggacggggctgcactccaaccgtctgcacctgctcttcccccacccctgtgggacctcatcttcacgtgccatgtgtgctgaaggcccagggcccagcagggggcagtggcacctgttgacggaaaagccgaggtgcttaccaatggaccttctggcccgccctcccctgtacttgtcgggcattcagggccccgacctgtgcctacccgca

SEQ ID NO: 234 - DNA, 568 bp, Homo sapiens
  misc_feature (430)..(430): n is a, c, g, or t
caactgtgttcactagcaacctcaaacagacaccatggtgcacctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcanaaagtggtggctggtgtgggctaatgcctggccccacaagtatcactaagctcgctttcttgctgtccaatttctattaaaggttcctttgttccctaagtccaactactaaactgggggatattatgaagggccttg

SEQ ID NO: 235 - DNA, 511 bp, Homo sapiens
  misc_feature (261)..(261): n is a, c, g, or t
ttttttaatttcaccaaaatttgttgacgtcccttgatttgctgatagggacaataattaaatattttccacttgtttttataaaaactgtaatggtgatttgtttaacagatgttgacttagcaccttctctctttttttttttttttttttgagttggagtcttgctctgtcacccagctggagtgcagtggcacgatttcggctcactgcaacctccgcctcccaggttcgggcgcttctcctgcctcagcctcccanatagttgggattacaggtgcatgccgccacncctagctaatgttttttgtatcttggtananatggngtttcaccttgttgcccatgccgctcttgaactccttggcctcccaaagtgttaggattacaggcgtgagccactgtgcctggccccaatttancaccttactgggtgctgaggctgtgagccatagtagaatgcatgtgatccagggccttgctgaattcatgggctaatagggagcctgac

SEQ ID NO: 236 - DNA, 592 bp, Homo sapiens
  misc_feature (504)..(504): n is a, c, g, or t
tgagcgttgggctgtaggtcgctgtgctgtgtgatcccccagagccatgcccgagatagtggatacctgttcgttggcctctccggcttccgtctgccggaccaagcacctgcacctgcgctgcagcgtcgactttactcgccggacgctgaccgggactgctgctctcacggtccagtctcaggaggacaatctgcgcagcctggttttggatacaaaggaccttacaatagaaaaagtagtgatcaatggacaagaagtcaaatatgctcttggagaaagacaaagttacaagggatcgccaatggaaatctctcttcctatcgctttgagcaaaaatcaagaaattgttatagaaatttcttttgagacctctccaaaatcttctgctctccagtggctcactcctgaacagacttctgggaaggaacacccatatctctttagtcagtgccaggccatccactgcagagcaatccttccttgtcaggacactccttctgngaaattaacctatactgcagaggtgtctgtccctaaagaactggtggcacttatgagtgctattcgtgatggagaaacacctgaccca

SEQ ID NO: 237 - DNA, 572 bp, Homo sapiens
  misc_feature (5)..(5): n is a, c, g, or t
cttanaagagttgctcattcacacccacgcccttgcccaaggctggcccactcagagcgaaacttaacttttgtctggatgggaagagaagtaagtctaccccgaggttgccatgttgaagagtgagaggtccaagtgattctgtgcattgaaaccaagacaccccacccagaacacttcttccctccctcagcccaaaccaaaggctggggttctcatctccaagtggctgttctccaactttcccaagccgcttgcattccccagactggactactgtggcggttaggttagatttgaagacggggcccaggctgggtatgaacgggtgcagccctcttctcctcttcccccccacatctctcatgagagaggtagtggcatttccttctcagggagcttcaatgggaaaggtctcgaaagcttcaggaggagcagaataccaacgcagggggatggctgtaacgatctcaccgtctcctaacctcagtcccttttttgagagtgaatggtggagggtgggaaagggacccaaatttgtagatctctttgtctgggggaggggaangatg

SEQ ID NO: 238 - DNA, 482 bp, Homo sapiens
ttaaaacaggcgcaggggtaaaaatgagaatgaatctgaaaaaagagagttggtgtttaaagaggatggacaagagtatgctcaggtaatcaaaatgttgggaaatggacgattggaagcattgtgttttgatggtgtaaagaggttatgccatatcagagggaaattgagaaaaaaggtttggataaatacatcagacattatattggttggtctacgggactatcaggataacaaagctgatgtaattttaaagtacaatgcagatgaagctagaagcctgaaggcatatggcgagcttccagaacatgctaaaatcaatgaaacagacacatttggtcctggagatgatgatgaaatccagtttgacgatattggagatgatgatgaagacattgatgatatctaaattgaaccaagtgtttttacatgacaagttctctgaggatggttctacagttgggattttggccatcatcaac

SEQ ID NO: 239 - DNA, 545 bp, Homo sapiens
tttgaaggcaaagagggattaatctgtgctggcatcatgtaaggagacttgatagataagaaaaagctttacctaagttttgaagaataggtttttcataatggaaaatttaagggaaaaatctccaaaaaagtgctactcaagttttatccatttgtatttccaacacagcctaggacagtacctgcacatagtaggtgattaataaaaatttagaaagcattaatactaaagaggaaaaatagcaatggcaagaaaacacatgtagggaacacatgtagccaaaaaataatatataatcagagaaataataggacttctggaaaaaaaagatgagatcagattggttaggatctttactaacatgacaagagcatgaattttttttctgtagataataagtatgaaagaattttagcttaaaaattagcataatttggatccacatatgcaaatcaatgaatgtaattcataatataaacagaactaaacacaaaaaccacgtgattatctcaatagacacagaaaaggccttcaaaaaaatt

SEQ ID NO: 240 - DNA, 624 bp, Homo sapiens; identical to SEQ ID NO: 209

SEQ ID NO: 241 - DNA, 421 bp, Homo sapiens
caagatgacaaagaaaagaaggaacaatggtcgtgccaaaaagggccgcggccacgtgcagcctattcgctgcactaactgtgcccgatgcgtgcccaaggacaaggccattaagaaattcgtcattcgaaacatagtggaggccgcagcagtcagggacatttctgaagcgagcgtcttcgatgcctatgtgcttcccaagctgtatgtgaagctacattactgtgtgagttgtgcaattcacagcaaagtagtcaggaatcgatctcgtgaagcccgcaaggaccgaacacccccaccccgatttagacctgcgggtgctgccccacgtcccccaccaaagcccatgtaaggagctgagttcttaaagactgaagacaggctattctctggagaaaaataaaatggaaattgtacttaa

SEQ ID NO: 242 - DNA, 539 bp, Homo sapiens
tgcttggccctctacctcctgccctcttcctgttcatctcccaaccactgcactcttgatttttataccacacagaaggtaagaaaattctaggaaccctaaggatcaatcctctccattttcactcaaatgcctggggcccagctctgcaatgactgactccagggcctctttcctcactgccagcatagaagtcaggggagccagctgggccctgcggtcaggaaggttctcatttttggagcattccctgagcccagatcataggagcagctgtccctggtgggacacaggagtcatgactcctaccctccaccctccacacccaccaggcatttagcagtctgtcctatgcaagacagatgaattctcagccaggatacctcaaggcaggcaaaggtgagtggagggaaaattcacaaacattcagggtgtgtggtgctggcatcaccatggccaaatccaagaggtcttcctggaagagggcccaaactggaaccaaaagaatgctgtcagcagttggaatagagctgtgaatt

SEQ ID NO: 243 - DNA, 397 bp, Homo sapiens
ctttccaagaggaatcctcggcagataaactggactgtcctctacagaaggaagcacaaaaagggacagtcggaagaaattcaaaagaaaagaacccgccgagcagtcaaattccagagggccattactggtgcatctcttgctgatataatggccaagaggaatcagaaacctgaagttagaaaggctcaacgagaacaagctatcagggctgctaaggaagcaaaaaaggctaagcaagcatctaaaaagactgcaatggctgctgctaaggcacctacaaaggcagcacctaagcaaaagattgtgaagcctgtgaaagtttcagctccccgagttggtggaaaacgctaaactggcagattagatttttaaataaagattggattataactct

SEQ ID NO: 244 - DNA, 542 bp, Homo sapiens
  misc_feature (535)..(535): n is a, c, g, or t
ctttgatagagaagaaaattctcctaggatacaagagcctcaacattttaaagattttctgcatctcaaaagcgtaggctccttgctgggcaaggtgagcctctgtgagtcctcataggaccgagcaaatctgattcaccccagaaaatccaatatcgaagctgagctttggcctgagcgggttccatttcctccccagatcctatttaggaagtgtctcctgacaacctccaaaaggtgctaacatgcaacgttctgaagggttattgctcaaaaacaagattttccttgtggtcaagactctgcgagcctcgaacacgatgaatccgctcgaatgggcttgggctttgcccgggtggcgcacgctcacacgctggaagcacagctttgacgatctccacacacgcacaggcacacacgccacagatgatgccggctcattctcagggggtgtctaagttctgctttaaatatttaccccctaattgtacaaacaataggggcatgagcctggtactcgataaatggggacttncttaaaa

SEQ ID NO: 245 - DNA, 649 bp, Homo sapiens
  misc_feature (615)..(615): n is a, c, g, or t
ctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggaaatccaggagctgcaggctcagattcaggaacagcatgtccaaatcgatgtggatgtttccaagcctgacctcacggctgccttgcgtgacgtacgtancaatatgaaagtgtggctgccaaaaaccttgcag

SEQ ID NO: 246 - DNA, 600 bp, Homo sapiens
gagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcagggtgctccacaggtagctctaggagggctggcaactta

SEQ ID NO: 247 - DNA, 331 bp, Homo sapiens
cgaatgtgcaggtttgttacataggtatatatatgccatgatggaaatatttatttttttaagcgtaattttgccaaataataaaaacagaaggaaattgagattagagggaggtgtttaaagagaggttatagagtagaagatttgatgctggagaggttaaggtgcaataagaatttagggagaaatgttgttcattattggagggtaaatgatgtggtgcctgaggtctgtacgttacctcttaacaatttctgtccttcagatggaaactctttaacttctcgtaaaagtcatatacctatataataaagctactgatttccaaaaa

SEQ ID NO: 248 - DNA, 41 bp, Homo sapiens
aaaaaaaaaaaaaaaaaaaaaaaaaaaaaaaaaaaaaaaaa

SEQ ID NO: 249 - DNA, 425 bp, Homo sapiens
caaaaaaacgaagaaaagtgacgacagtctgagggacttatgggagatcatcaagtgaaccactatatgtgtaatgtaagtcttggaatgagaagagagaaggagaaggaggagagagcttatttgtagaaataatggctgaaaacatcccaaactttcctttttttgaggaaagaaataggcatacaagttcaagaaactcaaggaactccagagaggacaattctaaagacaccccctctaacatacattataatcaaattgtcaaaagtaaaatacaaagagaatcttttaaattgacaagagaaaagcagctggtcacgttcaagggagttctataagaatttcagcagatttctcagcagaaaccttgcaggccaacaggcagtgggatgatacattcaaagtgcaaaaaaaaaaaaaaa

SEQ ID NO: 250 - DNA, 633 bp, Homo sapiens
  misc_feature (16)..(16): n is a, c, g, or t
cgagagtttaccagtngcctaataatgcaataaaaaatgctttgagatagctaacngcccataaaacaaactcaaattgcttataaagtttcttcccatgttcccatttgatgaaaagtcttacatcacatataactgggaagcaggggtccctcctcaattttcagacattttgaaaggatgacagttctgtttgttagatgagtaaacctctatattcataagttctaaaatccttcattatgagggattcaaagtatttataaaaacactgccctctaaaaatttcctcagatctgaagtatggncttggncctgaatatacagtgttatcctatgtttaaaagggtgatccagacatgagacgcaactagttggtgcataagaaggccccacttggctatttcatatctacctacaattgaccaaaaaaaattttttaggccagcaattattatttagcttcgctctttctagtgcaagaaactgcaggctggatcagtagttcaacagctaaacagtcataaaatagtcattggcatgttaaatttctttcaatgcttcaaagataaattccaattctatttacttattcattgngacngnattactaaacaggtaaggatgggaata

SEQ ID NO: 251 - DNA, 251 bp, Homo sapiens
ctttgggaggccgaggcgggcggatcacttgaggtcaggggttcgagaccagtctggccaacatggtgaaaccccaactctactaaaaatacaaaagttagccaagtgtggtggcaagtgcctgtaatcccagctactcgggaggctgagacaggagaatcactttgaacctgggaggcggaggttgcagtgagccaagatcgtgccactgcacttcagcctgggcaacagagcaagattccgtccatctc

SEQ ID NO: 252 - DNA, 593 bp, Homo sapiens
  misc_feature (157)..(157): n is a, c, g, or t
ctttcttcagccttgcagacacctaaacatcatgtaattacctaaggaattcccaagtgcctcttccaggttatacgtgtaaatagctgtttttatgcaagattagttagatactgctctttacaggatgagtggtgttgtctttggctgggggggncttaaatgtgtttctaatgtgtgtgtcaaataattacctgttaaacagactgccaatctggctgaagccaatgcttctgaagaagataaaattaaagcaatgatgtcgcaatctggccatgaatacgacccaatcaattacatgaagaaacctctaggtccaccacctccatcttacacgtgtttccgttgtggtaaacctggacattatattaagaattgcccaacaaatggggataaaaactttgaatctggtcctaggattaaaaagagcactggaattcccagaagtttcatgatggaagtgaaagatcctaatatgaaaggtgcaatgcttaccaacactggaaaatatgcaatccaactatagatgcagaagcatatgcaattgggaagaaagagaaacctccttnttaccagagagccatcttntttct

SEQ ID NO: 253 - DNA, 211 bp, Homo sapiens
gttgtgactcgttggcatgtgatctgaagttcctgccctgcagctgacgagccagtgtttcaataattaaaaacaactcaactcactgtcctcctgccttgaatttgatcattgcgctttgcatgtatgtatcacaataccacatgtaccccataaatatgtacaaagattatgtgtcaataaaaaacaaaaattaaaatcccaattttta

SEQ ID NO: 254 - DNA, 247 bp, Homo sapiens
gttgctagtagcggcaggaagatgtcaggctcactttcctctgattcccgaaatggggggaacctctaaccataaaggaatggtagaacagtccattcctcggatcagagaaaaatgcagacatggtgtcacctggatttttttctgcccatgaatgttgccagtcagtacctgtcctccttgtttctctatttttggttatgaatgttggggttaccacctgcatttaggggaaaattgtgttctg

SEQ ID NO: 255 - DNA, 244 bp, Homo sapiens
gtccccgggaatcgcggccgcgtcgacggtttattttcagtgcttgaagatacattcacaaatacttggtttgggaagacaccgtttaattttaagttaacttgcatgttgtaaatgcgttttatgtttaaataaagaggaaaattttttgaaaaaaaaaaaaaaaaaaaaaaaaaaaaaaaaaaaaaaaaaaaaaaaaaaaaaaaaaaaaaaaaaaaaaaaaaaaaaaaaaaaaaaaattttt

SEQ ID NO: 256 - DNA, 433 bp, Homo sapiens
tagaggcctgaataggtagacaatggcagcagcgtttttaatcacagtcctattcatgccctaattcgggagtgatgattaaaggacattagagggagcactttgacatctgatcctttgaactgacgtctgtgcaggctgcactccatagagctcacttggccaaactgatttccttaaataaagtgctgtgatttccaatgtaggaaatattacattagagcctattgaaatgattaggaattgaggagcttttctttaggtgggaatgtggtgtatgctgtatactcacaaaagtgagatcattaatattgcatgtactactttgaatatcagggaccacagagaaatagcatgagaaacgccttcctgcagtcatgcacttaaaatgaatatgaacaaaaatgtggaactctgctgtcatagctctccg

SEQ ID NO: 257 - DNA, 380 bp, Homo sapiens
ggggcttctgctgagggggcaggcggagcttgaggaaaccgcagataagtttttttctctttgaaagatagagattaatacaactcttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagttta

SEQ ID NO: 258 - DNA, 572 bp, Homo sapiens
  misc_feature (523)..(523): n is a, c, g, or t
gacctttgagaaaattaatttaaatcctagaactttgggtgaaccgaagaaatttataatatttgtttagttaataacagataaaaaggaaagattcaagcctattggatgagaatttgtacattattttagagctaataataatggttttcagtttagtgaggatttaaaaaatgtttttgaatcaaactttttttctttataatcctttttaactaactcaggaaataaggtattatgaaatccacacactgttacctccttaaagtatgaggatacttcccactgtttggtccactagtggctgattattttgtttgtggattatttgtaattttctttttaattcttccttaaagagcatggcatttggagtcacagacctatatttgaatcctgtcatttactagcgttttgaccttgaacaattatgctcagagtctcagttttttcttgtaaagtgatgatgatactacttaactcacagggttgtagtgaagatcaaatgagatcatgtctgtanaacaccctgcccggcactcaataagtattaataggaacccatatacctc

SEQ ID NO: 259 - DNA, 477 bp, Homo sapiens
  misc_feature (299)..(299): n is a, c, g, or t
tgtttttattttttaaaaggtataaacaccaaaaaaaaaattaacattgtatgaagatggaaaataagaagatgcactttctgtaactttgtctaaggatttaaattactaacttatgaactccaatttgaattgaacttaactatcggctttcttactggtaaaattatatggtttattttaaatgcgtacatattgaccaatggcctctgaaaaagcacattttagatactgaaattgaaggaaagaaaatgcatcttcaaacattttttggaatctcaccacatatactttgttanatttgtgtattgtagggtgtttgttttgtatttttgtattgtatatgaactttttttaaatgtgacagttaaacacatctttaaaagcatagtcacagacaaaagcatacagtataaaaatttccttgaaaactcctacaatattatatttggaggcagcttcagactgttttattgg

SEQ ID NO: 260 - DNA, 256 bp, Homo sapiens
  misc_feature (208)..(208): n is a, c, g, or t
ctctggcacacattagttcctcttatattacattgatataagcaagtcatatggatttatctgagtgtaaggagagctggaaaaaatagtttctagcaggtcagccacctcccagtgagggctgcataccatagaaggggagaatgaattttgggaaaacaggtaattatctctgtcacagaaggggatgaaaagtatggtagttacncaagttanacatctgtatggaaaataccacttggttctacaaatgngg

SEQ ID NO: 261 - DNA, 627 bp, Homo sapiens
  misc_feature (591)..(591): n is a, c, g, or t
gcctcccgggttcagggatttctcctgcctcagcctcctgagtggctgcattgcaggcacctgccaccacgccttgcaaatttttgtgtttttagtggagatggggttttgccatgttggccaggctggtctcggactcctgacctcaggtgatccgcccgcctcagcctcccagagggctgggattacaggcgtgagccactgtgcctggccccaagttttgcatcttttaatgccctctgaacaaatacatagagaaaactctcagaacaattaaaacctgcagagcaacagtgtcctccatgtcttaggtttcaagtttgcctctaaaattctaatccatatttttctacttctcagataatttatgtgtgtgtactcttcctagacgtacaagagactttttaatgctaaatatttgtcagtgcttaacaaaaactcaatttcacattactcatattgtttttgttttaattgaatgtgaattaaatttttattagttatttgatttggaatgttatgtatgccattaacactattaggggaatctctagcatttctgtatttttaaagaatttgattcttttgtanattctgcctgtgtggcattttaaacatgtgtgacat

SEQ ID NO: 262 - DNA, 345 bp, Homo sapiens
accggcgacatggccaaacgtaccaagaaagtcgggatcgtcggtaaatacgggacccgctatggggcctccctccggaaaatggtgaagaaaattgaaatcagccagcacgccaagtacacttgctctttctgtggcaaaaccaagatgaagagacgagctgtggggatctggcactgtggttcctgcatgaagacagtggctggcggtgcctggacgtacaataccacttccgctgtcacggtaaagtccgccatcagaagactgaaggagttgaaagaccagtagacgctcctctactctttgagacatcactggcctataataaatgggttaatttatgta

SEQ ID NO: 263 - DNA, 252 bp, Homo sapiens
ataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaaaattgatcagggaccatgaaaagaaacttgtgcttcaccgaagaaaaatatctaaacatcgaaaaacttaaatattatggaaaaaaaacattgcaaaatataaaat

SEQ ID NO: 264 - DNA, 294 bp, Homo sapiens
  misc_feature (15)..(15): n is a, c, g, or t
ttacttttaaccagngaaattgacctgcccgtgaanaggcgggcntgacacagcaagacgagaagaccctatggagctttaatttattaatgcaaacggtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctatt

SEQ ID NO: 265 - DNA, 370 bp, Homo sapiens
ggctgattcctgagctataaaagcataattgctttatattttggatcattttttactgggggcggacttggggggggttgcatacaaagataacatatatatccaactttctgaaatgaaatgtttttagattactttttcaactgtaaataatgtacatttaatgtcacaagaaaaaaatgtcttctgcaaattttctagtataacagaaatttttgtagatgaaaaaaatcattatgtttagaggtctaatgctatgttttcatattacagagtgaatttgtatttaaacaaaaatttaaattttggaatcctctaaacatttttgtatctttaattggtttattattaaataaatcatataaaaatt

SEQ ID NO: 266 - DNA, 353 bp, Homo sapiens
caggaagtcacctgggattggctgcctcacccactcacagtgccatccctgccccaggcctcccagtggcaattccaaacctgggtccctccctgagctctctgccttctgctctgtctttaatgctaccaatgggtattggggatcgaggggtgatgtgtgggttacctgaaagaaactacaccctacctccaccaccttaccctcacctggagagcagttatttcagaaccattctacctggcattttatcttatttagctgacagaccacctccacagtacatccaccctaactctataaatgttgatggtaatacagcattatctatcaccaataacccttcagcacta

SEQ ID NO: 267 - DNA, 433 bp, Homo sapiens
  misc_feature (229)..(229): n is a, c, g, or t
caggaagtcacctgggattggctgcctcacccactcacagtgccatccctgccccaggcctcccagtggcaattccaaacctgggtccctccctgagctctctgccttctgctctgtctttaatgctaccaatgggtattggggatcgaggggtgatgtgtgggttacctgaaagaaactacaccctacctccaccaccttaccctcacctggagagcagttatttcanaaccattctacctggcattttatcttatttagctgacagaccacctccacagtacatccaccctaactctataaatgttgatggtaatacagcattatctatcaccaataacccttcagcactagatccctatcagtccaatggaaatgttggattanaaccaggcattgtttcaatanactctcgctctgtgaacacacatgg

SEQ ID NO: 268 - DNA, 683 bp, Homo sapiens
  misc_feature (124)..(124): n is a, c, g, or t
gggttttctttcggaagcgcgccttgtgttggtacccgggaattcgcggccgcgtcgactgctaaacagaatactgctattttgagagagtcaagactctttcttaagggccaagaaagccacntgnnccctnggnctaatctggctgagtagtcagttataaaagccntaatngcttnntntttggnntcntttttnncnggggncggncttggggggggttgcntccaaagatancatntntttccaactttntnaannnaanngttttaaaatcccttttccnccngaaaananngccctttaagngccncaaaaaaaaanngtnttctgcannttttctantatnacaaannttttngtagaanaaaaatttttttttagnggctaccctttntttnttanncannggagtttntttttacaaaaaaaaaanattgggncccctccacaaccttgggtctntaatnggggggtttttaaataaancntntntaaatcccccnnnnnnnnncnnnnnnnnnccnnnnnnnnnnnnnncccnnnnaaaaaatttttnctcccccnccctttttcttcctgccggccccaatttaagcccnggcgcttggggcaaatccccctttagngggggggtttanaaaaaccnggggcggggntttaaaaccncggggnnnggggaa

SEQ ID NO: 269 - DNA, 177 bp, Homo sapiens
  misc_feature (118)..(118): n is a, c, g, or t
acctctagcatcaccagtattagaggcaccgcctgcccagtgacacatgtttaacggccgcggtaccctaaccgtgcaaaggtagcataatcacttgttccttaattagggacctgtntgaatggctccacnagggttcacttgtctcttacttttaaccagtgaaattgacctgcc

SEQ ID NO: 270 - DNA, 591 bp, Homo sapiens
  misc_feature (23)..(23): n is a, c, g, or t
gtatagaaaataatgtccccagngcatagaaaaaatgagtctctgggccagtgaatacaaaacatcatgtcgagaatcattggaagatatacagagttcgtatttcagctttgtttatccttcctgttaagagcctctgagtttttagttttaaaaggatgaaaagcttatgcaacatgctcagcaggagcttcatcaacgatatatgtcagatctaaaggtatattttcattctgtaattatgttacataaaagcaatgtaaatcagaataaatatgttagaccagaataaaattaattatattctggtcttcaaaggacacacagaacagatatcagcagaatcacttaatacttcatagaacaaaaatcactcaaaacctgtttataaccaaagaattcatgaaaaagaaagcctttgccatttgtcttagaaagttattttttaaaaaaaaatcatacttactattagtatctatggaagtatatgtaacaatttttatgtaaaggtcatctttctgtgatagtgaaaaaatatgtctttactaagttgaaatgaatactttctgnctttgctaatggatagttatt

SEQ ID NO: 271 - DNA, 329 bp, Homo sapiens
  misc_feature (199)..(199): n is a, c, g, or t
ctcaattctactaaaaagccccccaagaaaagcgaatgagaaaacagagtcatcctctgcacagcaagtagcagtgtcacgccttagcgcttccagctccagctcagattccagctcctcctcttcctcgtcgtcgtcttcagacaccagtgattcagactcaggctaaggggtcaggccagatggggcaggaaggctncgcaggaccggacccctagaccaccctgccccacctgccccttccccctttgctgtgacacttcttcatctcacccccccctgcccccctctaggagagctggctctgcagtgggggagggatgcaggga

SEQ ID NO: 272 - DNA, 688 bp, Homo sapiens
  misc_feature (2)..(2): n is a, c, g, or t
gnancntttcctntcgnaaancgcgccttgtgttggtacccgggaattcgcggccgcgtcgacaaaaaaaaaaaaaaaaaaaaaaaaaaaaantntagactcgancaagcttatgcangcntgcggccgcaattcgagctcggccgacttggccaattcgccctatagngagtcgtattacaattcactggccgtcgttttacaacgtcgngactgggaaaaccctggcgttacccaacttaatcgccttgcagcacatccccctttcgccagctggcgtaatancgaanaggcccgcaccgatcgcccttcccaacagttgcgcagcctgaatggcgaanggaaattgtaagcgttaatattttgttaaaattcgcgttaaatttttgttaaatcagctcattttttaaccaataggccgaaatcggcaaaatcccttataaatcaaaagaatagaccgagatagggttgagngttgttccagtttggaacaanagtccactnttaaagaacgnggactccaacgtcaaagggcgaaaaaccgtctatcagggcgatggcccactacgtgaaccatcnccctaatcaagttttttggggtcgaggngccgtaaagcactaaatcggaaccctaaagggagcccccgatttaaagcttgacggggaaagcccggcgaacgtggcgaaa

SEQ ID NO: 273 - DNA, 271 bp, Homo sapiens
  misc_feature (239)..(239): n is a, c, g, or t
cacctgcagtccaagtacatcggcacgggccacgccgacaccaccaagtgggagtggctggtgaaccaacaccgcgactcgtactgctcctacatgggccacttcgaccttctcaactacttcgccattgcggagaatgagagcaaagcgcgagtccgcttcaacttgatggaaaagatgcttcagccttgtggaccgccagccgacaagcccgaggaaaactgaaactttgcttaacnaccgaatggnggggancttttccaacgntttt

SEQ ID NO: 274 - DNA, 213 bp, Homo sapiens
  misc_feature (15)..(15): n is a, c, g, or t
ttggtttcatactgntggggnttgaatgntccctncaacactnatgttganacttaatccctaatgnggcaatactgaaaggtggggcctttgagatgtgattggatcgtaaggctgtgccttcattcatgggttaatggattaatgggttatcacaggaatgggactggtggctttataagaagaggaaaagagaactgagcttgcatgccc

SEQ ID NO: 275 - DNA, 545 bp, Homo sapiens
  misc_feature (8)..(8): n is a, c, g, or t
gtggaagngacatcgtctttaaaccctgcgtggcaatccctgacgcaccgccgtgatgcccanggaagacagggcgacctggaagtccaactacttccttaagatcatccaactattggatgattatccgaaatgtttcattgtgggagcagacaatgtgggctccaagcagatgcagcagatccgcatgtcccttcncgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactganatcagggacatgttgctggccaataaggtgccagctgctgcccgtgctggtgccattgccccatgtgaagtcactgtgccagcccagaacactggtctcgggcccgataagacctcctttttccaggctttaggtatcaccactaaaatctccaggggcaccattgaaatcctgagtgatgtgcactgatcaagactgg

SEQ ID NO: 276 - DNA, 260 bp, Homo sapiens
  misc_feature (35)..(35): n is a, c, g, or t
ggaaagggccattttattgcctaaaaccacctggnttttnaggtaacagttccaacatgtccttttttgaatagctgttctaattattatatattcagctgattaataggagtacttgataggtggactgtgtcaggtagcctcaggcaatcctacttcaacaagctgtcagggagccatgccatgcttctttatgacataggtgaatttgataggctcactagcagaacatgggatcacaaggtggaaccnttccnttt

SEQ ID NO: 277 - DNA, 603 bp, Homo sapiens
gaccccttccttacaccttatacaaaaaaactgaaactggaccccttccttacaccttatacaaaaattaactcaattttattatgttgtattaaattaagttgggtttaattaagatggattaaagacttaattataagacctaaaaccataaaaaccctagaagaaaacctaggccataccattcaggacacgggtatgggcaaagacttcataactaaaacaccaaaagcaatggcaacgaagtccaaatagacaaattggacctgattaaactaaagagcttcagcacagcagaagagactatcgtcagagtgaacaggcaacccacagaatggaagaaaattcttgcaatctatccatctgacaaggggctaatatccaaaatctacaaagaacttaaacaaatttacaaggaaaaacacaaacaaccccatcaaaaagtgggctaaggatgtgaacagacacttctcaaaagaaaacatttatgcagccaacaaacatgaaaaaaagttcatcatcactgctcattagagacatgcaaatcaaaaccacaatgagatcccatcccacaccagttagaatggcaatcattaaaaatgt

SEQ ID NO: 278 - DNA, 268 bp, Homo sapiens
tttatgtgtttttgcttggggggcgctgggcctagcccagagtagtgcttgctccccctgccttgtcccaccagggaggcagcagactcaggccctccatggtcctctttgtcattttgttgacatgcattcctccttttgtcatcttgttggggggaggggattaaccaaaggccaccctgactttgtttttgtggacacacaataaaagccccgtttatttgtaaaaaaaaaaaaaaaaaaaaaaaaaaaaaaaaaaaaaaaaaaa

SEQ ID NO: 279 - DNA, 569 bp, Homo sapiens
ctttagccagcctgatcagaaaaaaacaaaagaagaggaaagacgtagattaccaacatcaagaatgtgagttatgatatcactacagactctccaggtattaaaagcataattagagaatgatatgagcagctatatgcaaataagttcaacattggacaaatggacaaatttcttgaaagataaattatgaaatttcattctgaaagaactacatgaccttaattgtcttacatctattaaataagtggaaattgtagtttagaaactttcccacaaagaaaactctaggcccagatggcatcaaaataatattcagatgaatgaaatggagaaaggatagccttttcaacaaatggtggtggaacaattggatttccatatgcaaaaaaatagagatggacgcagaggtgtgtgcttaggaggctgaggtgagaggattgtttgaggccagcctgggcaacatagcaagaccccatttcaaaaacaaaaataaagaacttgtagccttaccttgtgccatattatgaaaatgtatcataggcttaaatgtgaaacgtaaaacaaaa

SEQ ID NO: 280 - DNA, 492 bp, Homo sapiens
cgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaatactaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgc

SEQ ID NO: 281 - DNA, 355 bp, Homo sapiens
cgaaaagcaaatataacttgccactaaccaagatcacctctgcaaaaagaaatgaaaacaacttttggcaggattctgtttcatctgacagaattcagaagcaggaaaaaaagccttttaaaaataccgagaacattaaaaattcgcatttgaagaaatcagcatttctaactgaagtgagccaaaaggaaaattatgctggggcaaagtttagtgatccaccttctcctagtgttcttccaaagcctcctagtcactggatgggaagcactgttgaaaattccaaccaaaacagggagctgatggcagtacacttaaaaacgctcctcaaagttcaaacttagatttcagattt

SEQ ID NO: 282 - DNA, 619 bp, Homo sapiens
ccggtctctacacaatatatagaaatctgggcatggtggtgcctggctgtagtctcagctacctagttgggtgaggtgggagagtcgcttgagtcctggaggttgaggctgtagtgagccagggctgcaccactgcattccagcctgggtaacagagtgagaccctgtctcaaaaagaaaaaaaaaaattgctaattttaacaaatcacaaaactgactcaggcaagttgtctgactcaaaagcccttgaaaaaccatcaaagacagtagaatgttaactggtcatttacgtaaaatagtgttcattaaatttttggttcatttaggataatcattttaaatgagactgtatttgagactgtatacacatacatatacatgtttacacacatatacgtacaatatatgtacattctatctaaaagatcatacatgtgtgtacatatatgtttttaaaagtcaaactgacatattaatggaaacagtgcttacatctctggtagtgattttctattagcagcagccctacatatgctgcgtctctgaacagcatgtcagtgccatgactgtctaaacatgcaaatatgactgacagactcttgagacagctttcaccttg

SEQ ID NO: 283 - DNA, 328 bp, Homo sapiens
  misc_feature (7)..(7): n is a, c, g, or t
aattcgnggccgcgtcnncctangaggcaccaggaaatcccgcggggtggcccatgcagaccaggcgcacgtggctcatggggcanaattgccaaggacagctcacgacagtgccaccttctcaccattccagccaaggagagatgtgacgttggaactgctctggcacttctgtcaagcctcccccgccccaattgccttgagatctctgctctttgtcagagatttgcaaagactcacgtttttgttgttttctcatcattccattgtgatactaagaaactaagaagcttaatgaaaagaaataaaatgcctatgttgttgttct

SEQ ID NO: 284 - DNA, 563 bp, Homo sapiens
ctagaacccatgactcctaggtcttatactgcaaccacagtatcagcaaataatctttcataaggggattattctctgattaacaggaaatacaggaatttaatttgtgaacacgctaggtagaagcagaaacccaaatccaaatccaaatttaaacatttaaaattcattctataactaagatctaacagtcattttcttcccagtaagaaataaccaaagcatgctaaaaatcactggactaaattggtgtcaaaactgccacattgccaggcatgggggggtcatacttgtaatcccagcactttgggaggccgaggtgggaaaattgcttgaggccaggagttcgaaaccagcctgggcaacacagtgagaccccatctccacaaaaaaaaaaaattaaaaaacaaaacaaaacattagctgggcatggtggtacacgcctgtagtcccagctactcaggagcctgaagtgagaggatcactgaagcccaggaggtagagctatgactgtagtgagctatgactgtgccactacactccacctgggtgacaggggactc

SEQ ID NO: 285 - DNA, 257 bp, Homo sapiens
  misc_feature (102)..(102): n is a, c, g, or t
cgacttccatttgtattaatggaatactaagtccctctgtgatttctgaaccaagctattcctaggcctgagttttattttgttgacacagaaataaattanaaggccaagcgtggtggcatgtgcctgtagtcctagttgctgaggtaagaggattgcttgagcccaggagttcaaggctgcagcaagctttgattgcgccactgcactccagccttggcgacagactaagacgctgtctcaaaaaaaaacaaaaa

SEQ ID NO: 286 - DNA, 602 bp, Homo sapiens
  misc_feature (57)..(57): n is a, c, g, or t
ggttatcaatgagattaagagacaactagagtaaaaacaaaagaaaagaaaagaaangaaaacaacagaagctctattaactgacctctaaccaatacaacaggttaactgatgttctccattctgtatataaaaatcccagtggacacccacaacacaggcttcaggcttgtaggacactttctagttcatctgagcacttttgttctcagcagttgagctgtatacttagcaacatttggtgcttccaaacccatttgtgcctgtagcacttactattgaaatacataatttaattaaatattatataaaggaatggaatacgagttggacaagaaaaagagttaaatctgaaggttaggtaaaaagagcaacttcttttctctgttttgcaggttggcaaaatcatttaaaaacaattggaagtattatatgttctgcattaagttgtcattttacttaaaaactaggcatcaaagatgatgcataataaatttagtgtatgcaagaatgactgcttgggacctcaatatatgaattcttaatccaaggaaagtccttggccttacatttaaaagtcggcaaataagtgtacgttcatt

SEQ ID NO: 287 - DNA, 306 bp, Homo sapiens
  misc_feature (250)..(250): n is a, c, g, or t
gaacatttaaaaataatgcaaataaggctgggcgtgggggctcacacctgtaatcccagcactttgggaggccgaggcaggcagatcacgaggtcaggagattgagaccatcctggctaacacagtgaaaccctgtctctacttaaaaaataaaaaaattagccaggcgtggtggtgggcgcctgtagtcccagctactcaggaggctgaggcaggagaatggtgtgaacccgggaggcggagcttgcantgagctgagatcgtgccactgcactccagcctgagcgacagagcgagactctgtct

SEQ ID NO: 288 - DNA, 419 bp, Homo sapiens
  misc_feature (216)..(216): n is a, c, g, or t
tcattagaatccaagctttgaaaatttctgattaatgctcatgtatttctttatctttgtttttccttgtgaagaaagactttcaccactgtctgagtgatgatgctgttgataaggatgatgtcgatgactactatattgcatctctcaggaacagctgatgggaagggaggggctgctgagttcccttgttctagctagcagcacgctcctcanagagggggccgagttacagacagcagccgcattctcatgcaaaattagttttaaactgctagtgtgggcatcggtaccttttgcctgggtgataccgaagaattgttgaggatttagtatgctccgtagagacagttcagccagtcatttctgcattggagagacttctcatactttctttgaagactcatagaaagctggat

SEQ ID NO: 289 - DNA, 626 bp, Homo sapiens
  misc_feature (13)..(13): n is a, c, g, or t
attaaggtttgtncccaacaagaatagatgtaattagaaaaaantgncttccttacctattgcctctgatntttacttgcttaaattttttttattgnaaatccagaaaaagnggatttagagaacaacactaactcccacctaatctatgacaganatgtacaananagtacctgtgaaaaatgtgaaagnatntgaaaaatgtaacctttggcagcctgagcatagtcaaccagaaaaactatctgaattaaaataattggtccataggtactattttatttggtccataaggattattttttcaacttttttttcaagtgtattattatgtcatttcccacgtaggttactgatacctgaagactttttncacctttaaccttnctcgttgaggagctttgtantctaataaaagagaaatataagtaaatgttagatatatgggnggataatggtaactatgtgcttaaagaggtataaaagaagggtagggagcagataagacaaaggaagggctatattataangaagaatattccaagtagggaagagaaaaagatatgttatccatataatattttatgtgcagtagagaacatgttctatagaanagacagaagatg

SEQ ID NO: 290 - DNA, 623 bp, Homo sapiens
  misc_feature (576)..(576): n is a, c, g, or t
cttgagcccaggaattccagcctgggcaatatagtaagactccgtctctacaaaagatacaaaaattagccagatgtggtggtgcgtgcctgtagttccagatactggaaagactgaggcaggaggattgcttgagcatgggaagttgaggctgcaatgagctgtgattacgccactacactccagcctgggcaacagagtaagatcttgtctcaaaaaaaaaattgaattcagctaaaaataataaaattttaaaataattttaaaaagccctcaacagctttgtttttctctccttgccagcttctctgcagcctatagcctgcaggctggctgctgcgagccaggacaagcggtgggaaatgcaatcacagcgtgaaatctctgtgttcagagacacgcaggaagcaggtgaaccatgaagggccaacacatgcccccagttagcagggtgtagagaccggggcagggctttcttcttccttctgggttataaatatccatgtcctgccatttgaagctgcaagtggcacacatggatgctggacaggcgctcgcactttctgggcagggcanggggctcaaaggcaggacagctgggcaaaagcaccttgcgtgggccc

SEQ ID NO: 291 - DNA, 579 bp, Homo sapiens
ctttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaaggtaattaaaattatcaaattggtgcttgatttctgcttttaaaatggtttatggaagaaaatatgattaaagttttgtattgttttccttcctatagaagatggagccagaatggcatgctaagttttttcttttctttagtgttatatatgacttctcctcaattgtcacccattgatctttaccactgttaataatggatgatattcaaaataccttatttcagtgattctaaggcaccattgattagaaactgcattattatttatgtgtccctaaaagctacctattaagctgttacacccaccatttttctgttaagaaaatcctgatttcagaa

SEQ ID NO: 292 - DNA, 709 bp, Homo sapiens
  misc_feature (3)..(4): n is a, c, g, or t
gtnntcctctcggaacgcgccttntgtagccaggtgctaccagaccnaatacacggttgttccagcttgcgcattcaccgatggcgtagatatccggatcggaagtctggcaggaatcattaatgacaatacccccacgcggagcaacgtccagaccacactgggttgccagcttatcgcgcggacggataccggtagagaagacgataaagtcgacttccagttcgctgccgtcggcaaaacgcatggttttacgcgcttcaacaccttcctgcacaatctcaagggtgtttttgctggtgtgaacgcgcacgcccatactttcgattttgcgacgcagctgctcgccgcccatctgatcaagctgttctgccatcagcataggggcaaattcgataacgtgggtttcaatacctaagtttttcagcgcgcctgcggcttccagacctaacaggccgcaattcgagctcggccgacttggccaattcgccctatagtgagtcgtattacaattcactggccgtcgttttacaacgtcgtgactgggaaaaccctggcgttacccaacttaatcgccttgcagcacatccccctttcgccagctggcgtaatagcgaaagaggcccgcacccgatcgccctttccaacagttgcgcacctgaatggcgaatggaaattgtaagcgttaatattttgttaaaattcgcgt

SEQ ID NO: 293 - DNA, 471 bp, Homo sapiens
ctgcgcagaccagacttcgctcgtactcgtgcgcctcgcttcgcttttcctccgcaaccatgtctgacaaacccgatatggctgagatcgagaaattcgataagtcgaaactgaagaagacagagacgcaagagaaaaatccactgccttccaaagaaacgattgaacaggagaagcaagcaggcgaatcgtaatgaggcgtgcgccgccaatatgcactgtacattccacaagcattgccttcttattttacttcttttagctgtttaactttgtaagatgcaaagaggttggatcaagtttaaatgactgtgctgcccctttcacatcaaagaactactgacaacgaaggccgcgcctgcctttcccatctgtctatctatctggctggcagggaaggaaagaacttgcatgttggtgaaggaagaagtggggtggaagaagtgggggtgggacgacagtgaaatctaa

SEQ ID NO: 294 - DNA, 495 bp, Homo sapiens
cttgtattcaagaactactgtaatgcattagtggtctggcttcattttgtatgatgccagatccttaatttacccagcacaatcatttcagtagtttcctatggctcctgcaaaaatgcaaacagaaaccaccacaggaacagccccttgctgcctcctgttgctgaggtagtagtcgctaaagaaaattgaaggctccttacaatctatatttgaaaactagaacttctgtagaaacacacagatcccgatcttagaagttgtacaggacaatctggtaaaactgacataattgtgatttattaacatgaattaaaatgcccaaccagtgcttcagtgtgacagtatatttaaaataaaaaagaaattaaaggtcatatactgtactactttcacaaagatccacagttttgcaaaagacttgtcatatgtacaatgctatatatcaaatgagaaaagctgtaagcaattatatacgcaaaagaaatggcagta

SEQ ID NO: 295 - DNA, 531 bp, Homo sapiens
ttccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaggtaaactacccaaccacatgccacgtaatatttcagccattcagaggaaactgttttctctttatttgcttatatgttaatatggtttttaaattggtaacttttatatagtatggtaacagtatgttaatacacacatacatatgcacacatgctttgggtccttccataatacttttatatttgtaaatcaatgtttttggagcaatcccaagtttaagggaaatatttttgtaaa

SEQ ID NO: 296 - DNA, 496 bp, Homo sapiens
caaccctctctcctcagcgcttcttctttcttggtttgatcctgactgctgtcatggcgtgccctctggagaaggccctggatgtgatggtgtccaccttccacaagtactcgggcaaagagggtgacaagttcaagctcaacaagtcagaactaaaggagctgctgacccgggagctgcccagcttcttggggaaaaggacagatgaagctgctttccagaagctgatgagcaacttggacagcaacagggacaacgaggtggacttccaagagtactgtgtcttcctgtcctgcatcgccatgatgtgtaacgaattctttgaaggcttcccagataagcagcccaggaagaaatgaaaactcctctgatgtggttggggggtctgccagctggggccctccctgtcgccagtgggcacttttttttttccaccctggctccttcaacacgtgcttgatgctgagcaaagttcaataaagattttgggaagttt

SEQ ID NO: 297 - DNA, 397 bp, Homo sapiens
cggatgtggtggcaggcgcctctagtcccagctactcggcaggctgaggtaggagaatggcttgaacccaggaggtggagctgacagtgagccgagatcgcgccactgcactccagcctgggcggcagagcgagactccatctcaaaaaaaaaaaaaaaaaaaatagactttgagaccagcctgaccaacatagtgaaacccgtcactactaaaaatacaaaaattacccgggcgtggtgacgggcgcctgtaatcccagctacttgggaggctgagacaggagaatcacttgaaccagggaggcggaggttgtagtgaactgaaatcgtgcccctgcactccagcctgggtaacaagagcgaaactccgtctcaaaaataaataaataaataaaat

SEQ ID NO: 298 - DNA, 293 bp, Homo sapiens
ccagctttttatggtgtttaatctaatacacttaagctgcagtcccaaaattaggggtccttcagtcttggagactataagggagcctctgcacccagggaaaatgttaccctttacaggggggaagggtaaaccagtagggaatacagtacaatcccaaccctactgggaggggcgggagggaggtgttgccgtcactgtattaagtcgatgttgggaaacgttttaacatctggagcctttgtgggtggaaatatgtctccagttacaactccgcagtggatgtgaagaag

SEQ ID NO: 299 - DNA, 452 bp, Homo sapiens
  misc_feature (210)..(210): n is a, c, g, or t
ggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttancaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaaggggttttaatgtanatt

SEQ ID NO: 300 - DNA, 480 bp, Homo sapiens
tggattcccgtcgtaacttaaagggaaactttcacaatgtccggagcccttgatgtcctgcaaatgaaggaggaggatgtccttaagttccttgcagcaggaacccacttaggtggcaccaatcttgacttccagatggaacagtacatctataaaaggaaaagtgatggcatctatatcataaatctcaagaggacctgggagaagcttctgctggcagctcgtgcaattgttgccattgaaaaccctgctgatgtcagtgttatatcctccaggaatactggccagagggctgtgctgaagtttgctgctgccactggagccactccaattgctggccgcttcactcctggaaccttcactaaccagatccaggcagccttccgggagccacggcttcttgtggttactgaccccagggctgaccaccagcctctcacggaggcatcttatgttaacctacctaccattgccctgtgt

SEQ ID NO: 301 - DNA, 498 bp, Homo sapiens
  misc_feature (426)..(426): n is a, c, g, or t
gtggtacatatacacaaaggaaaactatgtagccattaaaagaaaaggaactcctatcatttgtaacaacataaataaatctggaggagattaggctaaggtgaaataagccaggcacaaaaagacaactaccatatgatcttacttatacgtgtgtggaatctaaaaaggtggaatttacagaagcagagagtagaatggtgattaccagaggctggggagtgagggcaggaggttggagaaatgttggtcaaaggatacaaagtttcagttatacaggatgaataagttcaagagatctattgtacaacgtggtggctatagttgataacaatgtattgtgttcttgaaaaatgctgagagagtagattttaagtgttctcaccacaaaacataagtatgtgaggtaatgcatgtgttaattancttaatttagacatttcataatgtattatacatatttcaaaaccacgttgtacatgagaaagatacacaatt

SEQ ID NO: 302 - DNA, 474 bp, Homo sapiens
gcccagtcgacccatgttctcctttctacaccagcattagacgctgtcttcacagatttggaaatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactatacccgatcgcattcaggtccttcgcaacatggtcactgtgcagacctgagcaaccccaccaagtccttg

SEQ ID NO: 303 - DNA, 535 bp, Homo sapiens
  misc_feature (492)..(492): n is a, c, g, or t
ctgtaacagagattccttttttcaataatcttaattcaaaagcattattagacttgaaagggtttgataatctcccagtccttagtaaagattgagagaggctggagcagttttcagttttaaatgagtctgcagttaatatcaaatgtgagtttgggactgcctggcaacatttatatttcttattcagaacccttgatgagactatttttaaacatactagtctgctgatagaaagcactatacatcctattgtttctttctttccaaaatcagccttctgtctgtaacaaaaatgtactttatagagatggaggaaaaggtctaatactacatagccttaagtgtttctgtcattgttcaagtgtattttctgtaacagaaacatatttggaatgtttttcttttccccttataaattgtaattcctgaaatactgctgctttaaaaagtcccactgtcagattatattatctaacaattgaatattgnaaatatacttggcttacctctcaataaaagggtcttttctatt

SEQ ID NO: 304 - DNA, 464 bp, Homo sapiens
  misc_feature (427)..(427): n is a, c, g, or t
tccacccaccttgacctcccaaagtgctgggattataggcgtgagccacctcgcccagcccgatactaggacttatgcagaaaaaaccttgacatggaggaaagtaagatctaaataaatactgtattcatagattaaaagactcagcataataaatataccatttctccccagattgatgtacagatttaacacaattcctatcaagatcccagcaagatttttgtagatatgtaaaagattattcaaaaatgtaaaaggaaggacaaaggactagaatagataaaacaaaatggagaaagatttaataggaatcactgtaactgattttaagacatacagaacaataatagaaactgcttgtattagtccattttcacgctgctgataaagacatacctgagattggcaattacaaaggaaagangtttattggcttacagttcccatggctggggaggcct

SEQ ID NO: 305 - DNA, 588 bp, Homo sapiens
  misc_feature (511)..(511): n is a, c, g, or t
ctcctctgggttgaaacccgggcgccgccaagatgccggcttaccactcttctctcatggatcctgataccaaactcatcggaaacatggcactgttgcctatcagaagtcaattcaaaggacctgcccccagagagacaaaagatacagatattgtggatgaagccatctattacttcaaggccaatgtcttcttcaaaaactatgaaattaagaatgaagctgataggaccttgatatatataactctctacatttctgaatgtctgaagaaactgcaaaagtgcaattccaaaagccaaggtgagaaagaaatgtatacgctgggaatcactaattttcccattcctggagagcctggttttccacttaacgcaatttatgccaaacctgcaaacaaacaggaagatgaagtgatgagagcctatttacaacagctaaggcaagagactggactgagactttgtgagaaaagttttcgaccctcagaatgataaacccagcaagtggngggcttgctttgtgaagagacagttcatgaacaanagtctttcaggacctggacagtgaagggagcccgggcagcca

SEQ ID NO: 306 - DNA, 492 bp, Homo sapiens
  misc_feature (3)..(3): n is a, c, g, or t
cgnggccgcgtnaacttttgatcgtcagctggggctggcaggcacctaaatgggaagggtgatagcagtgtgttggggggagtttagggaacggtcctctaccgatagaggcagcanctcattggaatttcctcctgaagttgtcttgccccttgaatcctgcaggaaggctggcaaatggccatttcccttccacttgaatagagacccataactcaagtatctgcccttaagacaccacaggactgttcttcgcgggccctgcccctggatttgggagaggcagtccanctcacccaactaggctctgcanggggaccangagggatgggttgtgtccacaggaccagccagactgatgagggatgcggcaagcatattctcaccaccttctttcacgtttacaacanaccagcnttccctgtgtggcaggggttacattggtcaccgaggacctanaatcatggagtgctctggggatccgggcttgga

SEQ ID NO: 307 - DNA, 430 bp, Homo sapiens
  misc_feature (396)..(396): n is a, c, g, or t
tcagtgttgaattttgtcagacactttctctgcatcaattggtatgaccatgtgattttttttctgtagcctgttaatatggttaattttcaaatattgagctgattaattttcaaatattgagctctccttgcatctctggaataagtaccacttggtcgtggtatatatttcttttaatatattgctgaattctgtttgatcatgttttcttaaagactttcgtgtctgttttcatgatagatactggtctatagttttgttgtaatatcttggtttgattttgatatcaggataatgctaccttaatagaatgaattggagccaagtatggtggcaaatgcctatagtcctagctactcaggaggctgaggtggtggggactgcttgacccangagttcaaatctagcttgggcaatgtagcaagac

SEQ ID NO: 308 - DNA, 574 bp, Homo sapiens
tagaaggaatgactattcatgtccaaagtgaatggttttgtgcagtgaacaacacatggcgaggtactaactgagaaactttttcatgctttatgcctacctcttgtagttgttgcagagcaaatataaattgtaataagatagctaggccttgcagaaacaaacagaaaaacttaaaaaaaaatgatataagagctggagtctagtatttatatgaatctgtgagagataatttttttggtctcactgcaatgaaccaaaagcggctgagtttggtttttaattgtagccatgtattgaaggcatctttttgaccaactcttgttggttctgtcttgaaccattgttaatcactgtgctgtaattagtatagctaaatcttttccttccttgctcctcccccagcccaccccgtcttcccttaacattttttcagggggggttgggagtggtttcattttaatgtgagtggatgttttgatagttgtaaggaaaaaatgcatttcagacacatttcacacatgagctattttcttacacagtatgtcttattggtaataagaatgtaattcat

SEQ ID NO: 309 - DNA, 327 bp, Homo sapiens
  misc_feature (2)..(2): n is a, c, g, or t
cnttccntaagaatacaaaaaattagctgggcgtggtggcaggcgcctgtaatcccatctactcaggaagctgaggctggagaatcgcttgaacccgggaggcggaggttgcagtgagcagagatcacgccactgcagtccagcctgggcaacagtgcgagactctgtctcaaaaaaaaaataaataaattacctgggtgtggcagcgcgtgcctgtaatcccagctacccaggaggctgaggcaagagaactgcttgaacccaggaggcagaggttgcatggagctgagatggcgccactgcactccagtctggtgacagagtgag

SEQ ID NO: 310 - DNA, 273 bp, Homo sapiens
  misc_feature (37)..(37): n is a, c, g, or t
ctctctactaaaaatacaaaaattagctgggcacggnggtgcatgcctgtaaacccagctaccaggtactcgggaggctgaggcaggagaatcgcttgaaccagggagtcggaggttgcggcgagctgagatcatgccactgcactgcggcctggagacaagagcaagactccgtctcaaaaaaaaaaaaaaaaaaaaaaaaaaaagacntcacctaattgcagngngnggaccttatttggctnttaattcaaactattaaaaatgtgaacn

SEQ ID NO: 311 - DNA, 260 bp, Homo sapiens
cggggtctgtaccgggctggcctgtgcctatcacctcttatgcacacctcccaccccctgtattcccacccctggactggtggcccctgccttggggaaggtctccccatgtgcctgcaccaggagacagacagagaaggcagcaggcggcctttgttgctcagcaaggggctctgccctccctccttccttcttgcttctcatagccccggtgtgcggtgcatacacccccacctcctgcaataaaatagtagcatcgg

SEQ ID NO: 312 - DNA, 538 bp, Homo sapiens
  misc_feature (7)..(7): n is a, c, g, or t
ctgagtntagaaatgatgccattaatactgattgcaaaaacattacaactcagtactgcagctttcattcaaataggttatatgtataaactgagttcaacaatattgtatttgagatggtaaagttaaagaaatgcaataatgtaaataatacttaagaaaataagatctcaggaaactgtatatactctgtacttttatgcaactttatcagatcatttcagtatatgcatcaaggatatagtgtatatgacatgaactttgagtgcaaaaactgtactatgtaccttttgtttattttgctgtcaacatctaaataaaggtttttttgtttgttttttgtttttttaattgttttgttttaaagattgttttaattaattaaaaaattaattgttttaattaaacaattgtttaattgttttaaagtcgccaggctgaggcaggtgaatcacaagcttaggagttggaggctagcctgccaacatggtgaaaccccgtctctactaaaaatacaaaaaaattaactgggtgtggg

SEQ ID NO: 313 - DNA, 629 bp, Homo sapiens
  misc_feature (549)..(549): n is a, c, g, or t
cccatctgcaccagtacacaggcaggcattatcattcttcacctactttttaaatagtggcaacttgggattcattctggtgattctgaaccttgcctcatagcttaaagtataaaaaagattcaagagcagtgaggtttgttctttccagtgaatggtggactgagtggtgcgaggtggagggctaacaagaggaaagaactacattcttcagaatacagtgatgaaaattcattttgaaactcaaatattttcattttggatattctcctgtttttattaaaccagtgattacacctggccatccctctaaatgttctaggaaggcatgtctattgtgattttgatgaagacagaattatttttctctgtagaaacacagataccactttatcaggggaagttagtcaaatgaaatggaaattggtaaatggacaaaagctagctagtaaaaaggacgacccagcaacatgctttaaccccattgtatgtttgtggaaagagcatagtttaacatcttgagaaatttgggacataaaagttttcatnggtagacagttcatggcagtatatgaattgacataatggaaataatctgattttatttttacaactaacatcctttcccc

SEQ ID NO: 314 - DNA, 641 bp, Homo sapiens
  misc_feature (635)..(635): n is a, c, g, or t
ggaattccaagtgcttggggataatgatacctctgacctttcttccttttgggaagtacttgagtgtgcagctgcatgaggcctcagcaggagagagattttaggtccaagaagctataccagtaggacaaggcaggaaaatactacactttcaggatcaagcccctctgactctcatttggaaactggatgtttgctaagcacctgcttcttaaggatgccgagggatttaatgatactcccagaaacctggagagattaatggggcctatggagaagtgctctgaactcagtgttgggacttgaataaaattaaccattgtcatgttttcagaacaactaagctgttttatatttcatgtgcatgaaagccctagaactaagttgtgttatttccagaaatgaaatagatcccacagttagatgatgtggccattaggaagtaccaaatttataaaaatcactggaggtctgtctgagcagtacctaataaaatatagtatactgaaagtgaacagatactttgtctctttctttggctgcttgatctttatctgtgtctgccgtacagtgcacccttaaagtattctacaccagtgcttctcaaactggaaatgtgcatgtaagtcacccangggtct

SEQ ID NO: 315 - DNA, 645 bp, Homo sapiens
  misc_feature (215)..(215): n is a, c, g, or t
tgcatgcccatagtcccagctatttgggaggctgaggcaggaaaatcgcttgaacccgggagccagaggttgcagtgagccgagatcgcactccagcttggcgacagaacaagactctgtctcaaaaaaaaaaaaaaaagaaatcttgggatcctgaaccccttactcgaagggctaaggtagcatctcagcatgtcttattcgagacttcgtanaaccagacctgctgtttgtagatgttaattaatcaaacctttctctactcattctggaccagttaaggttttctccttctccgtatgagttttgattttcgtcctccttggttggagatcacactttggtctgctgctaagttggatgcctcccactgtctttccctaagtctagggcttcanaccccagtgtggggagagggactttcgtttcctgcccctcaccacatcagacacaggcaggcaagaataagatggccaaaaggccgatgaacttcttgacctagcctgggacattacctgttactaggtggacttcactgcctgtgaatggaagctgaagggctgtttttttggtttgtatttggacaggccaggcttanagagggagagaactgggctactcttcagcagtgatctttaaaatgcc

SEQ ID NO: 316 - DNA, 542 bp, Homo sapiens
cagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccacggccacagtcatggtggccacggccacagccactaatcaggaggccaggccaccctgcctctacccaaccagggccccggggcctgttatgtcaaactgtcttggctgtggggctaggggctggggccaaataaagtctctttcctc

SEQ ID NO: 317 - DNA, 583 bp, Homo sapiens
gaaccctgcggagggacttcaatcacatcaatgtagaactcagccttcttggaaagaaaaaaaagaggctccgggttgacaaatggtggggtaacagaaaggaactggctaccgttcggactatttgtagtcatgtacagaacatgatcaagggtgttacactgggcttccgttacaagatgaggtctgtgtatgctcacttccccatcaacgttgttatccaggagaatgggtctcttgttgaaatccgaaatttcttgggtgaaaaatacatccgcagggttcggatgagaccaggtgttgcttgttcagtatctcaagcccagaaagatgaattaatccttgaaggaaatgacattgagcttgtttcaaattcagcggctttgattcagcaagccacaacagttaaaaacaaggatatcaggaaatttttggatggtatctatgtctctgaaaaaggaactgttcagcaggctgatgaataagatctaagagttacctggctacagaaagaagatgccagatgacacttaagacctacttgtgatatttaaatgatgcaataaaagacctattgatttgg

SEQ ID NO: 318 - DNA, 424 bp, Homo sapiens
cttggctcctgtggaggcctgctgggaacgggacttctaaaaggaactatgtctggaaggctgtggtccaaggccatttttgctggctataagcggggtctccggaaccaaagggagcacacagctcttcttaaaattgaaggtgtttacgcccgagatgaaacagaattctatttgggcaagagatgcgcttatgtatataaagcaaagaacaacacagtcactcctggcggcaaaccaaacaaaaccagagtcatctggggaaaagtaactcgggcccatggaaacagtggcatggttcgtgccaaattccgaagcaatcttcctgctaaggccattggacacagaatccgagtgatgctgtacccctcaaggatttaaactaacgaaaaatcaataaataaatgtggatttgtgctcttgt

SEQ ID NO: 319 - DNA, 626 bp, Homo sapiens
  misc_feature (593)..(593): n is a, c, g, or t
gattttttttttttttttgagatggagtctttctctgtcgcccaggctggagtgcagtggtgaaatctcgactcactgcaacctccgtctcctgggttcaagcaattctcctgcctcagcctcctgagtagctgggattacaggcaccagccaccacgcccggctaatttttgtatttttagtagagacaggttttcaccatgttggctaggctgattttgaactcatgaccccaagtgatctgcccgcctcggcctcccaaagtgctggaattacaggtgtgagctaccactcccagccaatgattacatttataaggtaaaataacttgtgccaatctgtacaagtgaattcagatttaaaattttaattgtaaaaagatatccaggtgatatttctccctgaataatttagtttccttttctatttcttgatataaaagtactcagcattgaagtaattgctatcttcacatttcttcctatttgagctgtctaaataagtagtcctacatattttccccccaacacaaaaaacccagaaaagaattattttatactggatttttttggttgtagcaggaacctaaaggngccaattgtaacatgcatgttctttttggcaaa

SEQ ID NO: 320 - DNA, 618 bp, Homo sapiens
gtccatcctgcaggccacaagctctggatgaggaacttgaggcaagtcaccagcccctgatcatttcgcctaaaagagcaaggactagagttcctgacctccaggccagtccctgatccctgacctaatgttatcgcggaatgatgatatatgtatctacgggggcctggggctgggcgggctcctgcttctggcagtggtccttctgtccgcctgcctgtgttggctgcatcgaagagtaaagaggctggagaggagctgggcccagggctcctcagagcaggaactccactatgcatctctgcagaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggacagggtccccctgtggtccagccagtaaaaaccatggtccccccacttctgtgtctcagtcctctcagtcatctcgagcctccgttcaaaatgatcatcatcaaaacttatgtggctttttgacctttgaatagggaattttttaaaattttttaaaaatt

SEQ ID NO: 321 - DNA, 596 bp, Homo sapiens
ccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagttt

SEQ ID NO: 322 - DNA, 534 bp, Homo sapiens
  misc_feature (517)..(517): n is a, c, g, or t
gaggaaaggggagttaatatttagtggacagaatttcagttttacagatgaaaagagttctggagatagacggtgttgatagttgcacagcagtgtgaatgtgctcattgttaccgaacttaaaaatgtttaacatagtattatgtgatttttattttgccacttaaaaaaaaagaatgaagtactgatacatgctacaacatgggtgagctttaaatacattctgctcagtgaaataagccagatgcaaaagatcacatattatataatccacttatacgagatacctagaataggcaaattcatagagacagaaagtagaatagtggttcccaggggctggggacaagggggcagtgagagattgagagttattattaatgcgtacagagtttcagtttgggctgataaaaaagttctgaagatggatggtgatgatggttgtacatcaatgtgagtgtaattaccgccactgaactgcccttaaaaacgtttaaaagagtaaattttatgttgngtatattttaccataat

SEQ ID NO: 323 - DNA, 556 bp, Homo sapiens
tttttttttcataagaggcaagtacaagaaaaagcttaattactttaacttctaagtagtttggaatctaaataaataggagttaccaaatatatgcgcttctgtgaatagttttcccccacatgtttatttatatttttgcatctcatcaaacctaacagattctaaagtctctggtgataatgacaatatctgctacggagagactagcctgggggaagaggatctccctgaacaaggatagcggagttgctgcagctttcaaatgaagctggacatttagctgcgggggtagcaccctttgatcaaggcagcccaaagatgagtttcagggatgggactgacagaagagaaaagttcttcccagccctttctactttttctctttgtttctcaggcttctggccgtcttcagttttcacaagtttcactctcaaccctaaacagtacttctgtgaagtaccctttggcccctcgttttcagctcctaaactcacctggaaatagatgtcaatctaattttgggtctgactagtgcagtaggcatttttggtga

SEQ ID NO: 324 - DNA, 382 bp, Homo sapiens
  misc_feature (349)..(349): n is a, c, g, or t
ctcacacagaacaaaaatgaatgagtgtggctgtgtgccactatcactgtgtctacaaaaacagccagtgggcctgatttggcccttggctgcagtgcgcccgtctctgtttttgaggaataaaatcgcatcatttcatatggctaatgcaatttttttcccatctggaagcaacatctgattggactcatcttgtatggtgcttgttacagtctctgtaaatgggagagggtccgagaatagctcttcctgttttcatcaggactgtttttagggatggcaaagaagtcagtgtgtccagcctgtgtcctcctcaccacgtggctgattcctgaatctgcatgtgcancacntgccgttgtctggggcatgatctgtgtga

SEQ ID NO: 325 - DNA, 556 bp, Homo sapiens
  misc_feature (507)..(507): n is a, c, g, or t
cttttctctgggtatagatttaccctagcacctatctcattatattgaattttccagcatatttaaataaactattaattagtcacactatttcttaaaagtcacactatcaactaatcgtgaccgcaattatctaggggtgataatctgctgagtctactctttaaatacactgggacccagcatattgagttatattggcacagaaacttcactctgggtatagatttaccctagtaccttgccggcaggatcctattattcatggttgtacaagcaaggttcagggaagaggctggcacagagaaggtacctggtaactgttgtttgaggctgaattcagctcaactcagctccagtagagatggtgtccccttctctaccgtgttgagatagtgtgcagtcccttcctaagggctgttacccaccgcaataggacttgtcagcttcaacttttaaatttctctgctcccgctgggacccacccgcttcaaaaatcatcatggnggntttagcaccaatttagtaaacacaaactgtctgaaatattttggat

SEQ ID NO: 326 - DNA, 716 bp, Homo sapiens
  misc_feature (567)..(567): n is a, c, g, or t
gaacattcaagatagtgagaggaagaaaaagatatggctgtacgggaccgaggtctcttctattatcgcctcctcttagttggcattgatgaagttaagcggattctgtgtagccctaaatctgaccctactcttggacttttggaggatccggcagaaagacctgtgaatagctgggcctcagacttcaacacactggtgccagtgtatggcaaagcccactgggcaactatctctaaatgccagggggcagagcgttgtgacccagagcttcctaaaacttcatcctttgccgcatcaggacccttgattcctgaagagaacaaggagagggtacaagaactccctgattctggagccctcatgctagtccccaatcgccagcttactgctgattattttgagaaaacttggcttagccttaaagttgctcatcagcaagtgttgccttggcggggagaattccatcctgacaccctccagatggctcttcaagtagtgaacatccagaccatcgcaatgagtagggctgggtctcggccatggaaagcatacctcagtgctcangatgatactggctgtctgttcttaacagaactgctattggagcctggaaactcagaatgcagatcttttgtgaacaaaatgaagcaagaaccggagacnctgaatagttttatttctgtattaaaaactgngattggaacaattgaaga

SEQ ID NO: 327 - DNA, 664 bp, Homo sapiens
  misc_feature (586)..(586): n is a, c, g, or t
ccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaatgaggattctgaccttgactttgatatcagcaaattggaacagcagagcaaggtgcaaaacacaggacatggaaaaccaagagaaaagtccataatagacgagaaattcttccaactctctgaaatggaggcttatttagaaaacagagaaaaagaagaggaacgaaaagatgataatgatgatgagtcaggtaaaagttccagaaatgtgaacaacaaagatttttttgatccagttgaaagtgatgaagacatagcaagtgatcatgatgatgagctgggttcaaacaagatgatgaaattgctgaagaagaagcagaagaaggaagcatttctgaaatatgaatgaaaaaaattacatctttagaaaaagagttattagaaaaaagccttggcagccgtcngggggaagtgacgcacagaagagaccagagaatagcttcctggangagaccctgcactttacccatgctgctggatgg

SEQ ID NO: 328 - DNA, 641 bp, Homo sapiens
  misc_feature (522)..(522): n is a, c, g, or t
ccgggttttagtatttaaccaagagccttttaaatattgaaaacccatagttcagaaaatgttagtattgctgcccttcttcacataaatttttttttaaattatactattattttgcttaattttatattgggttaaaacaaccttcaagaaggttaactaggaaagaagacctttttgttttatttttactatttatatatagaagacaaatcagcatttggtgatagttttacatgaccagttatcaaacggtcatagtatgaagtgtgcagttgttcattattagtaaattatgtttgatttttaaactatttagtactaatagttgagatgaaaactgaagaaaaatgccaatgtgacgtttgtgtatagctagccttaaaaaacttcccatgtttttaggtgacttttttccccctcttagtactctggagaaacaatgaagatgggccatctcaattccagatgtaaacaaaaagtaatttttatttcaacatttaatgtaactgctattattgnggattcttgncttgngtattttctttcccttattcaagtaatatagaataactttccttaaaatgatttgatccaagatacgtcatttctgtattggcaaaatgccnctattaaagtgt

SEQ ID NO: 329 - DNA, 132 bp, Homo sapiens
gttaaagtgatacatttttataccaaatgtgtttatttttttgtgcaagtaatccttaaaattgcaattgtattaggtgttaaaataaagtttttaaaaaattaaaaaaaaaaaaaaaaaaaaaaaaaaaaa

SEQ ID NO: 330 - DNA, 666 bp, Homo sapiens
  misc_feature (623)..(623): n is a, c, g, or t
gacaaccttagccaaaccatttacccaaataaagtataggcgatagaaattgaaacctggcgcaatagatatagtaccgtaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaaatcccaaacatataactgaactcctcacacccaattggaccaatctatcaccctatagaagactaatgttagtataagtaacatgaaaacattcttctncgcataagcctgcgtcagattaaaacactgaactgacaattaa

SEQ ID NO: 331 - DNA, 370 bp, Homo sapiens
  misc_feature (67)..(67): n is a, c, g, or t
aaagagctcccaaatgctatatctattcaggggctctcaagaacaatggaatatcatcctgatttanaaaatttggatgaagatggatatactcaattacacttcgactctcaaagcaataccaggatagctgttgtttcanagaaaggatcgtgtgctgcatctcctccttggcgcctcattgctgtaattttgggaatcctatgcttggtaatactggtgatagctgtggtcctgggtaccatggctggtttcaaagctgtggaattcaaaggataaattaatgaagaaaacaagcggagctgaagaagaaagtacaatatggtgctgtcttcctaatgaaataaattcactaaatggacattaaaaa

SEQ ID NO: 332 - DNA, 741 bp, Homo sapiens
  misc_feature (612)..(612): n is a, c, g, or t
agactcgagcaagcttatgcatgcatgcggccgcaattcgagctcggccacttggccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttaaagcttgacggggaaagccggcgaacgtggcgagaaaggaagggaaaaaagccaaanggagccggcgctagggcctggcaagtgtacgggcacgctgcgcgtaaccacccacaccccgccgngcttaatgccccnttcagggcgcgtnctgatgccgnattttntcttacncatntgtgcnggntt

SEQ ID NO: 333 - DNA, 719 bp, Homo sapiens
taaaataatggcaaaaaacaaacaaaaaacaagttctctaaacagaaaggaaattactaaagaaggaatcttgaaataacaggaaagaggaaataccacagtaggcaacattatgggtaaataaaacagactttccttctttagtttcctaaaatatgtttgatgattaatgcaaaaattacaatattttcttatgtagcactaaaggtatgtagagaaaatatttaagataattgtactgtaagcgggagatgacagtgacataaaggcaacgtttttatacttcactcaaactttatgtattaatgtaatccataaagcaaccaaaaaagctatactaagtacattcaaaaacacaatagataaaccaaacaaaattctaaaggatgtacaagtaacccactggaagctgcaaaaaatgtaaacagaaactaaaaacagagaataaatgaaaaattaaaaacgaaatggcagacttaggccctaatatacaaattatcacattaaatataaatggtctaaatacaccaactgtaagacagagattagcaaagtcgatttaaaaacatgactcaactacgtgctgtctacaagaaactcacttcaaatataccaagataggaaggttgaaagtaaaacgatggaaaaagatgtatcatgtgaacattaatcaaaggaaagcaggggtggctatattaacatcaggtaaaataaacttt

SEQ ID NO: 334 - DNA, 603 bp, Homo sapiens
  misc_feature (6)..(6): n is a, c, g, or t
tgaggntggtcatgatgcanaagctactcaaatgcagtcggcttgtcctggctcttgccctcatcctggttctggaatcctcagttcaaggttatcctacgcggagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagacagccaggacttgggtcaacatggattagaagaggattttatgttataaaagaggattttcccaccttgacaccaggcaatgtagttagcatattttatgtaccatggntatatgattaatcttgggacaaagaattttatagaaatttttaaacatctgaaaa

SEQ ID NO: 335 - DNA, 71 bp, Homo sapiens
atttatctaatatttggtttaataaaatgtgaataatgaaaaaaaaaaaaaaaaaaaaaaaaaaaaaaaaa

SEQ ID NO: 336 - DNA, 622 bp, Homo sapiens
  misc_feature (617)..(617): n is a, c, g, or t
tttttttttattttttgagaatggagtcttgctctgccgtccaggctagagttcagtggtgcgatctcagctcactgccacctcacctcctaggttccagagattcttgtgcttcagcctcctcagtagttgagaatacaggaacacgccaccacgcctagctaatttttgtatttttagtagagatggggtttcaccatgttggccaggctggtctcaaactcctggcctaagtgacccacctgcctcagcctcccaaagtgctgggattataggcgtgagtcattgtccccagccggatgttttcatcttgatttgccttagtttctaaatctcatcctctccattttctcctgttagtagtcacagagaaccaaattctgtcaagttatgaaactaaagtctctcttccacaagtcttcctgtgttctgcctcaagtgaacttgaaagaacatcagtttgtgggaaggttgaagaccgaatgatctgctgggaaatcactgaggcattgccattctcttgaggaatttcattttcatcgaagtttcggtttatatccctttcttggtgagtactattgctgttatgtaaattaaatgagtcgtcatccttcttntgagc

SEQ ID NO: 337 - DNA, 501 bp, Homo sapiens
gtgaaatcactttcatggattattaatggatttaagagggcatcaatcagctcaactcaagatttcataatcatttttagtatttagattgtgcctcaaagttgtagtacctcacaatacctccactggtttcctgttgtaaaaaccttcagtgagtttgaccattgtgctcttggctcttgggctggagtaccgtggtgagggagtaaacactagaagtctttagtacaaaactgctctagggacacctggtgattcctacacaagtgatgtttatatttctcataaagagtcttccctatcccaaggtcttcatgatgccagtagccatatatgataaattatgttcagtgataacttagttatcagaaatcagctcagtggtcttccccgccatgattcacatttgatgagtttttaaaaatcaaagtgattttgaaaatctctaatggctcagaaaataaaaacatccagtttgtggatgactatatttagatttct

SEQ ID NO: 338 - DNA, 630 bp, Homo sapiens
ttgtgtttttaggactccttatctaaattaaggcagagaagttacagtatttatatctgcattaaatctcaattccagaaaaaccttttgaaaaattatttaatcctctggaaactattgatatgatacaggagaaattttcagaagtttattgaataatttaatatcatttaataggacactctggcttgtatataagcagatacgttactcagacttcttggctgtactctaaaataatatatgtactagtctcctaaatattactagctcacctttcaaaatgcatactaatatttcaatgtctttcttcaatttgaaaagctcttgaatatctacttgtgatagccctaagagctgagataattatttccaggaggttgaatccctgattcttaactgttcagcaatgcataagcaagagagaatatgacataagaggaccatttctacattagccattttttttcacaagatacctatgtgaatacagggcacctgggagggtaagtggaggactatttctaactatatttataagcacatactgatattggtgaatcaaaacctacagcagtgcttctcagatgggaagggagacaatgtgtaaggagatcaggaattcattag

SEQ ID NO: 339 - DNA, 122 bp, Homo sapiens
  misc_feature (4)..(4): n is a, c, g, or t
ccanaatccactctccagtctccctcccctgactccctctgctgtcctcccctctcacgagaataaagtgtcaagcaagaaaaaaaaaaaaaaaaaaaaaaaaaaaaaaaaaaaaaaaaaaa

SEQ ID NO: 340 - DNA, 640 bp, Homo sapiens
  misc_feature (110)..(110): n is a, c, g, or t
tttttttttttttttttttcagagtcacagatattgtatagctgaggtaagcattttacaacttttcagacacaagtaagtacataaatattattttacaaccaacaatntttaatatttccacattgaanaatagatgtgataattaaatcttttataaggttttaaaaagacatgaaacataaacctaattatacataaaagaaaagaattttaaacaagagcttattgngatgacattactcataacttttacctttaaaaccttttcttgggtagctattcaaaagtaaagaccacaagttttgttgcccanatttcttatgtttngtatatttaagctctttatttattgaacagatgngtcattaattcattnggagcattactattatcagtaaaatttgatttttttttcccctcagtcataggtaaatcagctccacctggaatttctaaggacccagttttagtcaatattttcaagtaatcatgacctcagaaatagtcttaattaagataacaaatattagccatcaaaatggaaccaagacaagattctaatgtttgtaaacagtcaatccatatttatgaatattagcatatattggngaatagttaaggcaaaagggtctagcag

SEQ ID NO: 341 - DNA, 667 bp, Homo sapiens
  misc_feature (505)..(505): n is a, c, g, or t
ttgtgtttttaggactccttatctaaattaaggcagagaagttacagtatttatatctgcattaaatctcaattccagaaaaaccttttgaaaaattatttaatcctctggaaactattgatatgatacaggagaaattttcagaagtttattgaataatttaatatcatttaataggacactctggcttgtatataagcagatacgttactcagacttcttggctgtactctaaaataatatatgtactagtctcctaaatattactagctcacctttcaaaatgcatactaatatttcaatgtctttcttcaatttgaaaagctcttgaatatctacttgtgatagccctaagagctgagataattatttccaggaggttgaatccctgattcttaactgttcagcaatgcataagcaagagagaatatgacataagaggaccatttctacattagccattttttttcacaagatacctatgtgaatacagggcacctggganggtaagtggaggactatttctaactatatttataagcacatactgatattgntgaatcaaaacctacagcagtgcttctcagatgggaagggagacaatgtgtaaggagatcaggaattcattagtcacctttcagatggtttaatgcatacagctgtaccg

SEQ ID NO: 342 - DNA, 591 bp, Homo sapiens
ggagtttgagcagatccttcaggagcggaatgaactcaaagccaaagtgttcctgctcaaggaggaactggcctacttccagcgggagctgctcacagaccaccgggtccccggccttctgctcgaggccatgaaggtggctgtccggaagcagcggaagaagatcaaggccaagatgttagggacaccagaggaagcagagagcagtgaggatgaggctggcccatggatcctgctctccgatgacaagggagaccatcccccacccccggagtccaaaatacagagtttctttggcctatggtatcggggtaaagctgaatcctctgaggatgagaccagcagccctgcacccagcaagctagggggagaagaggaggcccaaccacagtctccagctcctgatccgccctgttctgccctccacgaacacctttgtctgggggcctcagccgccccagaggcctgacttaggggtctggctgtggaaggatgtgtggcctcaaatgaggacagggctcccgccttcacagccctcgccaggggtctgccccaatcctggcctgcatcaggcaaggacggggtctcagc

SEQ ID NO: 343 - DNA, 642 bp, Homo sapiens
  misc_feature (441)..(441): n is a, c, g, or t
gcaagtcttcagtatgtacatttatcccctagaagaagaaaaattagttgtgcatgaaaaagaaacattaactgcaaagctaaatgctcacactctaaatcagtgctctccaaagtacagcaggcgggaaaagaaaatggtagatttttttcttccaattactttaacttattctttttaatggacacttcatacataaatatattcacaatatattaatatatacataatgtataagcatacatattgaatgtgcagtcaaaaaatgtactaatggaatgctctaccaaaacaagttcacgttcatctgtaaaatgggaataatatttttaaaaggcatacagtctgaacatttttagattattcataaaatctattcagaaagttaaactaaaaaatttaacgtatgcctataacaaattttgtacttaatgtaattgnttttcatcctgagatctaatatcctcgtttttaagtagagccacttgtttgctacagtttagtcaaaacgttaacattagatgggtaaagtaatatgaaatctttctactactccaaaatagaaaacagaacattaaaaagataaaaattcaaacatacttaccagtagattttcaactgngcaaaagctcattgcatggg

SEQ ID NO: 344 - DNA, 661 bp, Homo sapiens
  misc_feature (55)..(55): n is a, c, g, or t
gttttccaccgtgaagagaacatttcctctgggaatgacaaagccctcaggaacngcttttatttctattggaagatgcccatcatacttctggcaggataaaatgataaatttatttattcaacagatgatactcaattccctgctgttttactaaaggttctttacgttttatagaagctaaatttactgtcatagaaattgcaattgtagatgttactgtaatctagtcagaatatccttatccttctaaaataaaactagttaaaattattaacatacgtactgatattaatttttaagtttaatgctgccacgtgcttctgctaagaacatttatcactacaagtggcagaaaattccaaactcatcaaaaccaaactgttgcttcttccctgctttttcagaaaatgagaaaggatgactttattccaacatattctaaaagtattccaagaacactacctttattctaaattcgttattttcacaaaataaaggctgcagattgaaagataaaggattgctattaaagaacaaaagaaaacaaaaccgagagagaaggagagctagggaaatccctgcanaanaaccgaatanggtccctctattctgggccggggcctgaaactatgaaacaggccaacacagaatcttggca

SEQ ID NO: 345 - DNA, 661 bp, Homo sapiens
  misc_feature (142)..(142): n is a, c, g, or t
cctctgactcgctcagctcacccacgctgctggccctgtgagggggcagggaaggggaggcagccggcacccacaagtgccactgcccgagctggtgcattacagagaggagaaacacatcttccctagagggttcctgtanacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcanagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgnagtttttcttcaacatcaatggtcattgnaatgttactgatcatgcattggtgaggnggtctgaatgttctgacattaacaattttccat

SEQ ID NO: 346 - DNA, 115 bp, Homo sapiens
aaacttttgtggcaacagtgcactaatttggataatgtttgttcccaataaattaagagccaaattgtaaaaaaaaaaaaaaaaaaaaaaaaaaaaaaaaaaaaaaaaaaaaaaa

SEQ ID NO: 347 - DNA, 634 bp, Homo sapiens
  misc_feature (593)..(593): n is a, c, g, or t
gccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccaatccagctcggcaacagactcttccatggaggagatctttcagcacctgatggcctatcaccaggaggccagcaagcgcatctccagccacatccctttgatcatccagttcttcatgctccagacgtacggccagcagcttcaaaaggccatgctgcagctcctgcagggacaaggacacctacagctggctcctgaaggagcggagcgacaccagcgacaagcggaagttnctgaaggagcggcttgcacggctgacgcaggctcggcgccg

SEQ ID NO: 348 - DNA, 528 bp, Homo sapiens
  misc_feature (362)..(362): n is a, c, g, or t
gttgccgggtcctgtgataactctgtttaacattttgaggaactgttgaatggtttttcacagcagctgcctcattttttattcccatcagcagtacttcttggttctaatacctccacgttctcgccaacacttgttgttgtctgtaatttcgttgttagccatcccagtggggatgaagtagtatcttactgtggttttcagttgcgtttccctgataattaatgatggtgaacatcttttcatgttcttgttggccatttgtatgtcttcttgggaaaaaaaaaatgtctgttcaaatcctttacaaagtatttattttttatgtcaacaatataaccactcagtacactgctttttanacaatgatcttttaaaggtttgtttacaacatttagcacttgaaattttaaggttatgccctcaaaaaaattgctgagggagctaagctatgaagatgcaaaggcataanaattatacaatggactttgggggaatccagggaaagggtgggaggggggtgangga

SEQ ID NO: 349 - DNA, 573 bp, Homo sapiens
  misc_feature (54)..(54): n is a, c, g, or t
tcgactctgatttttttttctccttcctcgcagccgcgccagggagctcgcggngcgcggcccctgtcctccggcccgagatgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatanaaataaaagctgttcctgagggctttgtcattcccagaggaaatgttctcttcacggtggaaaacacagatccagagtgttactggcttacaaattggattgagactattcttgttcagtcctggtatccaatcacagtggccacaaatt

SEQ ID NO: 350 - DNA, 659 bp, Homo sapiens
  misc_feature (216)..(216): n is a, c, g, or t
tcatttacattaatactcaaaactgctcgattaagcaggtgctgttcttatcgccattttgcatatgatgagaaagggtaaggtcacccagctagtatttggctcacagcaggccttaagacttggtttgtgtgactcatcagtccacgctcctaaaaccactaagttgttctaccctttaatgttgaattaacattggatagtgttcaagtttanatgggtgggtgagggcccaaggacctttcaaactcagatctcttatttaataacctggtcccagatccattcctctgtcgaagaggaagtcatccttcagtggctattcattgtggggttaagagcgcagactatgaattcagtctttttgggtcccagtttgccagaccttgagtgagtgccccgagtttacttacttgtaaaggtaggtggaggtaatataattaaataaacttaaaaaactaattaaaaacaaaacaaatgaactaaggtcttaggatatctggcgtctattttgcgccaaatcacataatgtctattgttgtgtgttggactataggattgtcctttaacagggaagggtttatttctgtaatcaagtctgtcaatattatgaccatgttgataatagctacctttaattgagggcttccatgtgccaa

SEQ ID NO: 351 - DNA, 517 bp, Homo sapiens
  misc_feature (251)..(251): n is a, c, g, or t
tcagtggaaaagggcaggttgaatcaaggtgaatcaatctgaaattgagcacacctgcctgccatcgctgttccttcaactgagtgctgcacatcatgggctctgtctgtgagagaaaaatcccggtgcttggtgtccttgcatgacatggagttttgcatgtagatcaatttaaaatgtacctcttgtttacataatttgcataattttaaaagataatgttgccaaactttggaaatgttaatgttcanactgaaaatctccactacatgtaactttcttcctctggatcagtggcatggcttataatcccagccagtggtttgaactgttccagtgtcaactgccatgtgctctgcttcaagggggaactagccttttgtgaattttttgtacataagtatttgttacaaatattttagcaaatgctttctatttctcttgcttgtgcatatcttggctggcgttacagaaaaatagtgtaaacattatttccttaccggggaatgagggtttt

SEQ ID NO: 352 - DNA, 174 bp, Homo sapiens
agcacctggcacagagtagtagctaacacagatgttaattttgctgcgtcaaatgttttcactttgaatctctcttgagtattgttctccttattgattacatgatgacatcctgttttctctccctgacctttactgtttgtttagaaaaaaaaaaaaaaaaaaaaaaaaaaa

SEQ ID NO: 353 - DNA, 664 bp, Homo sapiens
  misc_feature (240)..(240): n is a, c, g, or t
cagagagcttgttccctccctccctgtgcatgcaaacaagagggcatgggagcacacagagagatggcagccacctacaagccaagaggagaagcctcacaatcaaactctcgctgctggcgagagtcttggactctgtcttggacttccagcctccagactgtgagaaacaaatttctgttgtttcagcttctcagtctctggtgttttgttattgcagcctgagaacacagctgtacnattatnagggaaacagaaaacactgatacttaacaatgctaatgcaattatttatttgcttttcagtctctacaaaacgttctaaaacactaatctaaatattaacagtaaaatatttgcataactaatggaaactaagaaatcatatgaccaatatttcacttattggtaatcttactctactgatttccccccagactgtgatttttgaacttccttgcctttctcctgtctttctgngtttattcatggaattccagttatctgggcttgaaattgcaggctctcctaacttaagcaaaatctgacagatcagcaaaatgagataaatgtttcttttttctttctgactgcattaaatcagatacaactcagcattaaaaagctatctttgnaaaatgntggtactaataaattagtctta

SEQ ID NO: 354 - DNA, 661 bp, Homo sapiens
  misc_feature (493)..(493): n is a, c, g, or t
ccagttccacattcagtgaagtcatgaacttgaaattggccatgatcaaaaagtatttaaatcacagaagttgcaaatgccacaaatcaaggtctttttctcttggagaacctgttaaacatttaccaactcacgaccgccatgcacccaatactgcaataggtctatagatgcagatactgtctccatgaatcttataggctagaaaggaaatagataagtagtcctaccagaagaacatgatgaaggcatttgtggtaaacagaatgatggccccccaaagatgtccacatcctaatccctgaagcctatgaatatactactttacttggcaaaagggactttgccacaggtttttaattaaggaccttgaaatagagagattatcctggataatccagatggccccagtgtaatcccaagggtcctcacaaagggtaggaaggagagccagagtcagagaaggagacgtagcaatggaggcagaggtcanagagagatctgcagatgctgctgtgttggctttgaaaatgaggaatgcaggtgacctcaangngctagatgatgcaaggaaacaaataatctcctatgaaccctaggatgggcattattatgagtcctattttataaacaaggaactgacntccagaaagataaatgc

SEQ ID NO: 355 - DNA, 626 bp, Homo sapiens
  misc_feature (55)..(55): n is a, c, g, or t
ggcagaggttgcagtgaactgagatcatgccattgcaatccagcctgggcaacangagtgagactccatctcaaaaaaaaaaaaaaaaagacaagagtntccactctaaacacttntattcaacatagtcctgaaagtcgtagccacagcaatttaacaagataaagcaataaaatgtattcaaatagaaaaagaggaagtcaaattatcttcactggngatataattctctacctgggaaacttcaccgaaaaagatttcaccaaaagatttctaagcctaaataatgacttcagcaaagtctcaccatacaaaatcaacatacacaaatgagtagcatttctgtgcaccaataatattcaagctgagaaaaaaagaacatggttctatttacaatagctacaaacaaaaaaatatgtacctagtaatacattaaatcaaggnggtaaaatatctntacaacaagaactacaaaactgctgaaaaaaaatagagacacgcaaataagtaaaaaggcactccatgctcatgaatttaaagaatcaatataattaaaatgtccgngctgcctaaagcaacttacagattaaaggctatttctctcaaactataaatgcaccttttta

SEQ ID NO: 356 - DNA, 585 bp, Homo sapiens
  misc_feature (317)..(317): n is a, c, g, or t
gtcattgctgggtggcgccagccctcagacttgcctctttgcagtaggaagaaggcctccccacataccttcccacactcatcaccttaagccagactcggtgtccagtgaatatgaccatctcttgcccattttctaatgagtgttttcattaatgagttataagaatgtggtgggtaaatctatgggctttgaactagtgaatcaacttggtttcagaatctggcactgctacttactagtgaatttaagcaagttatttcacctttcagagtgtcagttccctcatgcatacaaggaagataaaaaataatgtntacnaaagtattggagtaattaatacatggagaactacatgtaaagcgtttagcatgatgtctgacatattaagcatccaatattagtngcttgcagaattattagtaaaagagattgcttctgaaagccattccaattcttaaattttataatgccacatttgaggtcacctgaagtcgtgtataacatgtgtacatttttgcgatttattttttcaattcccanattaaaggcatagagatatcctagcnanggactccaagtgtg

SEQ ID NO: 357 - DNA, 560 bp, Homo sapiens
  misc_feature (477)..(477): n is a, c, g, or t
gtaattgcagcctgggcaacggagtgagagactgtctcaggaaaaaaaaaagaaaaaaaactactgaggtagttgaatatatcctccattccccatttgtggattagttagtaaatggggcatcttagggtttaaatatgtccagggtcactgaggatcagatcctagggttcctttgactcaaggcttttgtctcagcaaaacgtcaccttccagcaggaaggctttctcaggcaagtagcagggtggctactatgtatcgcttctttattttttcttttttaaaataatgcaggcaccgtgcgcataatttaaaaaatcagtgctaaaacccttaaaaaaaaaaagctgttctcatctcctgtctttcttttttttttctttttatttttttcttttattattattatactttaagttttagggtacatgtgcacaacgtgcaggtttgttacatatgtatacatgtgccatgtnggtgagctgcacccattaactcgtcatttagcattaggtatatctcctaatgctatccctcccccctcccccctttttttttt

SEQ ID NO: 358 - DNA, 645 bp, Homo sapiens
  misc_feature (76)..(76): n is a, c, g, or t
gggaatgtcttaggcactgggactgtaagtgcaaagaccctgtggcacaagggaatgttaattatctacctttcanaaactggaanaaggcctagcctagagcattgaaaacaataagggaaaggaggagtaaggctgganagataggaatggtttaaagtctttgttaaaaatttttttaaaaaaatctttatcacaagaagaggattggcntgatcaaatttgacttttaaaaanattacttgggttgggcatgatcaaatactacttagggagattagtttanatgataatggcattctggaccanagtggagtcagaggtgaaaagaggtagatattccanaattgagggatttgtgaggtgaaatcatttgttacagatattaaaggataaggagctttgtcaaaggggatcttaagtttctggtatggtaactgggttagagagccctggaacatgaccagctttaagggaagagagcttgagctctgttcttgttaagctcagtttgagatctttgtggaatcaagtggagaggtctaagcagggaactggcttggctaggctgtaaagatgaatctgagagtcccaagaatatggtaattattaataaaagccttaggtanatgaaattgttttggg

SEQ ID NO: 359 - DNA, 509 bp, Homo sapiens
gcaaatctacacatttgattaaatgatagggaactatgcacacacataatacatataatgctagtttcttggttttgatattgtaccatagttatgtaagatgtaaccattgggggaaactgggtgaaggctacatgagacctctctgtacttaatctttgcaacttatgtgaatctataattattccaaaataaaaagttttaaagaacctaagtatccttattactgagggtcatcgtgctagacagcaaggttgggccagagcttctagttatttaaaatactaaataccagcctgggcaacatagcaagaccctgcctctacaaaaagcaaaaaaattagctgggcatggtggtacatgcctgtggtcctagttactcttggaggagtctgaggtggggagcttgagcctaggagtttgaggccgcagtgagccttgattgtgtctctgtactccagtctgggccacagagcaagacccggtctctaaaaataaataaataaata

SEQ ID NO: 360 - DNA, 511 bp, Homo sapiens
  misc_feature (2)..(2): n is a, c, g, or t
antgcactccagcttggtgacagagggagactccatnttaaaaaaaaaaaaaaaaaaaaaaaagggagtagcttgaagccacatagtagttagtggtaaaggccaccccttttcccacaactcacaccagcaccacaagctagcctttntaatttccaagccagtgccctttcaacgcacacacccctgtgtcagttccctttctgctgcaagctctctggaggcagatactgttgagtccctggcctgcctatgagaacggctcatgatctctatttcttctgcttaatgaccatctcgaagtaacaagtttagcctaaaataaacttgctaagttagcaaaggaagtccttagcagccaccatttctcgattcctccatcacctcccctgcccctcaactccctcatttctcccaagatatgggctccaggctgggcgcggtggctcacgcctataatcctagcactttgggaggctgaggtgagcagatcactgaggtcaggagttcg

SEQ ID NO: 361 - DNA, 16 bp, Homo sapiens
  misc_feature (3)..(3): n is a, c, g, or t
tcnttcggaacgcgcc

SEQ ID NO: 362 - DNA, 579 bp, Homo sapiens
ctggagggatgggtaggattttgacaagagtggttgaaggtattctaattcacttagtacctacatgtgcgaggcagcatgaaggcaaaaaagcctggggcatgttcagagaatagcaagtattctagtttgagtggcacctggtacgtatataagggaatagtaaaagatctggctggaaaggaaaagtaggggcaggttacgaaggacctctgaaagtcagactgtggaactggaacttttatcaggaagcagtagttagttttttcaagcaaaagctaattagagttgatatttaggaggatgaatctaacagttgtgtgcaaggatgccttcaaactgagtgagactagtactggagactggttaagagactacaacaataacctgagtaagaattaatacaggcctgacctagttttgagtgagtaggattggaaacaagagttttaggtattataggatttatgcatataaaatggacttgacagaacttgaagaaagagaaagtgtcaaaaggacacagaaagtgaggcaggatatcttacaatgttaaaggaaaggaataatagaagttac

SEQ ID NO: 363 - DNA, 185 bp, Homo sapiens
ggaaacataagcttgtttcagtacactcacgctgtagattaattctgatattacatatctccatcagactttgtaccctctctcttccatcccttacccttaccgattaggttggtattacctaaaaatccatagaaaatgtccaggtgaattgccttatgctttctaccccataaggtataatt

SEQ ID NO: 364 - DNA, 649 bp, Homo sapiens
  misc_feature (649)..(649): n is a, c, g, or t
ctctgtggtgtgagaacacagtgggtgaccaaggctttccagatgaacccaaggaaagtgaaaaagctgatgctaataaccagacaacagaacctcagcttaagaaaggcagccaagtggaggcactcttcagttatgaggctacccaaccagaggacctggagtttcaggaaggggatataatcctggtgttatcaaaggtgaatgaagaatggctggaaggggagtgcaaagggaaggtgggcattttccccaaagtttttgttgaagactgcgcaactacagatttggaaagcactcggagagaagtctaggatgtttcacaaactacaaagctgaagaaaatgaagccctattacttgtttgtaagatttagcacccttctgctgtatactgtactgagacattacagtttggaagtgttaactatttattccctgttaaaatttaacctactagacaatgatgtgagtacccaggatgatttcctggggcacagtgggtgaggagatggggacaggtgaatggaggagttaggggagaggaaaagtggatggaagtgtctggaaagggcaccaaaaaagtcttccaggtctgatcctgtttcttgctctgagtgctagctaccactgtgtcacactgtaacatn

SEQ ID NO: 365 - DNA, 655 bp, Homo sapiens
  misc_feature (607)..(607): n is a, c, g, or t
ctcagctcttgcctggtcaccttgtggcttttaccatcctcatcccctgtgccacccacatcctgccacttctgcatggagttggggtggggccattggagaaaagaggttaaacaagcagtaatttacttgagtacagtctttgagccaatgaaatgccagtcatcatttcccaggggtacttgtcatcttgtcaacaacccgctgataatgctccttcaatgtgaatagcaaaagtagggagagacgctgaatgaagaagatgcctacccctcaggaagactgctgtccgcctccaggcctgcatgcacacacccatgcccacctgcacccccagcaccacgcccacactcactcgcacacacccacatgccagtgttttggggttggcagcctggacactgctgaggcaaacacaagtcatcaagcataattctcattctctccttctgtctctgttttagttacaggaatttggtcagtttagaggatttaataagtccgtggaaaatttgtttctgtctcttgctacccacgtgaaaagtaagtgcatgcttcatgatgtgttttcccactaccttccaggccagccgagcccactggccanggcctggcccggtgacctcggttgacactgtcctcangccactcactt

SEQ ID NO: 366 - DNA, 334 bp, Homo sapiens
  misc_feature (254)..(254): n is a, c, g, or t
cagaatttcatgtttatgctgcacaaggcctgtattttataatggtggctcttttggacgatgacttcctcgatggtgaaacttccagtaatctccctcatcatactgaaatgatatcagtatatcatcagaacaccatggagcttgtcatttgagggacacagcttgcttgtgtgcttgggaaagaagaggtttagcatggtttcaggtcagtgatgagtccaatgatctctgcaagttcccttagctctganaattctgatgtcatatgcacttctgccgccagagttgctgcttactggatgcgtaagaagaaaagaaaaaaaaaaaaaaa

SEQ ID NO: 367 - DNA, 582 bp, Homo sapiens
  misc_feature (506)..(506): n is a, c, g, or t
cttccattgggggtaaagatcaaactttaggcgagccaggtctgtatctccattcctgtctctgactgcttccctgtagggattgtctgcaagcgcacacctgcattttcttgtccacaagtctatgctctaactctgtcacctgcatggctgcaaattagcttccttcttcctgccctcttctctctagcttggattttgaatttgaatggcaggcatgggatgtccgtgtgtgtgtactgctgatgtgtacagccgcttgttagcgctctcattgtcttcaaatgtaagtcattttggctgggtgcggtggctcatgcgtataatcccacgctttgggaggctgaggtgagctgatcatttgaggttaggagttcgagaccagcctggccaacatggcaaaactccatctctaccaaaaatacaaaaattagctgggtatggtagtgcacgcctgtaatcccagctacttggaatgctgaagcaggagaattgcctgaacccangaggcggaggttgcggtgagccaagatcacgccactgcactccaacctgggtgacagagcaaggctgtgtctcaaa

SEQ ID NO: 368 - DNA, 530 bp, Homo sapiens
acctgacttcaaactatactacgaggctacagtaatcaaaacagcatggtactagtacaaaaacagaccaatggaacagaatagagatctcagaaataaaactgcacatctacaaccatctgatcttcaacaaacctgacaaaacgagcaatggggaaaggattccctatttaataaatggtgctgggagaactggctagccatgtgcagaaaattgaaactggaccccttccttacaccttatacaaaaattaactcaagatggattaaagacttaaatgtagaacccaaaacgataaaaaccctagaagaaaatctaggcaatatcattaaggacatagacatgggcaaaaatttcatgatgaaaacatcaaaagcaatggcaacaaaagcagaaactgacaaatgggcttctgcacagcaaaagaaactatcgtcagagtgaacagacaacctacagaatgggagacagtttttgcaatctatccatctgacaaaagtctaatatccagaatctacaaggaatttaa

SEQ ID NO: 369 - DNA, 653 bp, Homo sapiens
  misc_feature (596)..(596): n is a, c, g, or t
caaaaaacaagaattacccgggcttggtggtgcatgtctgtagtcctatctactcaggaggctgaggctgaaggatcacttgagcccaggagtttgaggctgcagtgagtgagccatgatcatgccagtgtactccagccttggcagactgagcaaaacttggtccctcgcaaaatgttgaagcccagttttcactattaacctgtatttcagtttccccatgctaactttgaaacactggggctggcctgagggtataaaggcttattcaaactcagtaatttaaacttaaaatcctaaggaacttcaaaaagtgtaatctagtccaaatggggcatcaattctaaagcatttgcttgtttgagcagattttctgtgtctgaggtatatagataacttatctttttatgactaaatccaagtccttagttcctgttggaattcaaaatcatatttaaaaattgatgctttgttctataattaatgctttgattgtataaataataagtattcttccaaatccctttttacagatgatgattctgataccgagacgtcaaatgacttgccaaaatttgcagatggaatcaaggccngaaacagaaatcagaactacctggntcccagtcctgtncttaaaattctaactcgac

SEQ ID NO: 370 - DNA, 595 bp, Homo sapiens
  misc_feature (43)..(43): n is a, c, g, or t
gagggtgtagaagagaagaagaaggaggttcctgctgtgccanaaacccttaagaaaaagcgaaggaatttcgcagagctgaagatcaagcgcctgagaaagaagtttgcccaaaagatgcttcgaaaggcaaggaggaagcttatctatgaaaaancaaagcactatcacaaggaatataggcagatgtacaaanctgaaattcgaatggcgaggatggcaagaaaagctggcaacttctatgtacctgcagaacccaaattggcgtttgtcatcagaatcagaggtatcaatggagtgagcccaaaggttcgaaaggtgttgcagcttcttcgccttcgtcaaatcttcaatggaacctttgtgaagctcaacaaggcttcgattaacatgctgaggattgtagagccatatattgcatgggggtaccccaatctgaagtcagtaaatgaactaatctacaagcgtggttatggcaaaatcaataagaagcgaattgctttgacagataacgctttgattgctcgatctcttggtaaatacngcatcatctgcatggaggatttgattcatgagatctatactgttggaaaac

SEQ ID NO: 371 - DNA, 651 bp, Homo sapiens
catttccagagtttatgtgaattgaattgaactatggttttatgttactgtcagtagaatgaagtacgaatatttgaaaaatacaccttcaacttcaaagtgattcttgacaaaaattataaggaatcattttggacacattttctggtagagccttgtaaaaattaaaaccaagtgttgttttcaagaagaactgtaatacataatcaggaatttgagtagggagattattttgttatttaaaattaaagtggctgtgtagttttaactttagtattgcaggtagagtaagcttacatgataacaaaaatcttggtcttagtgacttaatgattctgatatttattgattgattggttatcattccaaatattttaaaagataatagctggctgggtgcggtggctcatgcctgtaatcccagcactttgggaggccaggacgggcggatcacgaggtcaggagatcaagaccatcctggctaacacggtgaaaccccgtctctactaaaaatcaaaaaattagccgggtgtagtggcgggcacctgtagtcccagctactcaggaggctgaggcaggagaatggcatgaacctgggaggcggagcttgcagtgagctgaaatcgtgccactgcctccacctggcgacaa

SEQ ID NO: 372 - DNA, 531 bp, Homo sapiens
  misc_feature (243)..(243): n is a, c, g, or t
gtgaggtggggacttcattcattgtcctatttctatctccactttgtgcctggagagctttcaggggaggtggaggaggagggtctgccaagctactgcaacatctgtcacccactatacccagttacttgggggaggacagacactgtggtgtcattaaagttgtttgaaccaaagtggcggctgcatctttgtcccgatgctagccgtgccggtctcccatcatccgctcgccctcctttnccctgggctgcgcccacttgtcttcctggatatttgggggtgactcgccatgcttggcaccctctgcttcctggtgctgctctgactcgaagacgggacagtccctggtgcacatccagggaagaggagtgtcggtagttcttgcagtaggcactttatcaggacctgacctgttgctgggtgattttagtctctacaaacagaaagcgtttcaaagcgtcagctgtgggagcagagtgaccctttgctgatgctggggggaggggatctaaatcctcatttatctct

SEQ ID NO: 373 - DNA, 602 bp, Homo sapiens
  misc_feature (232)..(232): n is a, c, g, or t
ggcgcctgctggaggaggagagagctctgctggcatgagccacagtttcttgactggaggccatcaaccctcttggttgaggccttgttctgagccctgacatgtgcttgggcactggtgggcctgggcttctgaggtggcctcctgccctgatcagggaccctccccgctttcctgggcctctcagttgaacaaagcagcaaaacaaaggcagttttatatgaaagattanaagcctggaataatcaggctttttaaatgatgtaattcccactgtaatagcatagggattttggaagcagctgctggtggcttgggacatcagtggggccaagggttctctgtccctggttcaactgtgatttggctttcccgtgtctttcctggtgatgccttgtttggggttctgtgggtttgggtgggaagagggccatctgcctgaatgtaacctgctagctctccgaagccctgcgggcctgcttgtgtgaaccgtgtggacagtggtggccgcgctgtgcctgctcgtgttgcctacatgtccctggctgttgaggcgctgctttaacctgcacccctnccttgctcatanatgctccttttga

SEQ ID NO: 374 - DNA, 230 bp, Homo sapiens
  misc_feature (109)..(109): n is a, c, g, or t
tttgagaccagcctagccaacatggtgaaaccccatctctactaaaaatacaaaaattagccgggcgtggcggcacatgcctataatcccacttacttgggaggctgangtaggagaatcgcttgaacccananaggcagagtttgcagtgagccgagattgtgccattgcactccagcctgggcgacagagcgagactccatctaaaanaaaataaatgaataaaataa

SEQ ID NO: 375 - DNA, 483 bp, Homo sapiens
  misc_feature (1)..(2): n is a, c, g, or t
nncagatttttttttttttttcagngttagaccatctttcaattcctggaacaaacttaactttccatgatatgtattttttatacattgctggattttatttgctaatattttacttaggatttaattttctaagtngacctataattntcctgtataaaattgcatttgtcacattttagtatcaaggttgtcctancnccatgaaatggatttanaatggtttatgtaanataaagtacatttcttctaaaggtttgngtggattaactttcaaatctgccanagngngtttttttcctttttttttttttttcatttnaagggagngcaagtancttttcaaatnctgatttaatttttaaaatatttncaagtntntttanagtttttatttnttntngaangttaacatttttatanaaaanggtnttatctttttaaattctttgacatcagtttcttcanaattccttcttttaa

SEQ ID NO: 376 - DNA, 538 bp, Homo sapiens
gtcatatctcttcccagggaaagcaggagcccttctggagcccttcagcagggtcagggcccctcgtcttcccctcctttcccagagccatcttcccagtccaccatccccatcgtgggcattgttgctggcctggctgtcctagcagttgtggtcatcggagctgtggtcgctactgtgatgtgtaggaggaagagctcaggtagggaaggggtgaggggtggggtctgggttttcttgtcccactgggggtttcaagccccaggtagaagtgttccctgcctcattactgggaagcagcatccacacaggggctaacgcagcctgggaccctgtgtgccagcacttactcttttgtgcagcacatgtgacaatgaaggacggatgtatcaccttgatggttgtggtgttggggtcctgatttcagcattcatgagtcaggggaaggtccctgctaaggacagaccttaggagggcagttggtccaggacccacacttgctttcctcgtgtttcctgatcctgccttgggtctgtag

SEQ ID NO: 377 - DNA, 440 bp, Homo sapiens
tggccatccttttccccccaaacacacccccttaacctatctcttgggacttagcccgaccctccctctcatttcccattaagtctgagaggcaagagctaggttaggcaaggaggtggttggccagagatggggaacagccaggtgccccagtcctctgatttttcctccatcctgcttaccacctccctgggtacttacagccttctcttgggaacagccggggccaggactgggtcacctatgagctgaatcagcatctcctcctgagtcccagggcccctgcagttcccagtctcttctgtcctgcagcccttgcctctttcccacaggttccactttatatccaccttttccttttgttcaatttttatttttattttttttattattaaatgatgtggtctatggaaaaaaaaataaaaatctgacttagtttt

SEQ ID NO: 378 - DNA, 513 bp, Homo sapiens
ggaacccagtgtattacctgctggaaccaaggaaactaacaatgtaggttactagtgaataccccaatggtttctccaattatgcccatgccaccaaaacaataaaacaaaattctctaacactgcaaagagtgagccatgcctgttaacactgtaaagaatgtaacatgtgggggacacacaggggcagatgggatggtttagtttaggattttattagtgcatgccctaccctctgggggaacgtcccatctgaggttttcttctcggtggggggatttaacttctgtcctagggaaaacagtgtctgatgaggagtgtttccaacacaggctacatgaattcccctataccagtgcgaaagcagccaggagtccccgttggaaaagaacaatgccactctcttttatgtatcttggttctgcaactcatttgttgtaagtagggttaatcgagtatcaggttcacagtatcctgcccttattattttatgattcactgactcaagttcca

SEQ ID NO: 379 - DNA, 646 bp, Homo sapiens
  misc_feature (485)..(485): n is a, c, g, or t
gagagtgaaaaaattctggtacaaattgggaaattagtatataacaacatagtgttaaattcaatgggaaaagtttaataagaggatttggtatcaactggctgtccaaagataaaaatggaccgtcctatcacatacaaaattgttttttagataaagatttaaatacaggcactccttcatttgcgtggtgcaccttgaggtgttgcagaaatgatgagagctgaaactgcaaagcaattttaatactttatctgttggaaatcttatagttttcctgtgaccgttaaaattttcattaaactattaaaaacacccatgactggtcacaaatgtattgggaaatggaaaagaattaatacactaaaaatacaaaaaatagaaaatatttaaaattatctaaaaatttgaaacattagaaaaattgagaactaggcagggcgtggtggctcacatctgtaattttagccctttgggaggctgangcaggtggatcacctgangtcaggagttcgagaccagcctgccaacgtggggaaaccccgtctctactgaaaatacaaaaattanccgggcatggtggcacaagcctgtaatncttgctnaccaggangctgaggcaggagaatcacttgaacccangang

SEQ ID NO: 380 - DNA, 362 bp, Homo sapiens
  misc_feature (51)..(51): n is a, c, g, or t
gtttcacatgagaaggtagtattatgtacagtgaccttgtttaaagtgtcngtttaatgttaccactaaggccctgccccagctttatcacctgagcactaacaagtgctgtgtggagttcagtccatgctggtaactnttgagtattcagtgggtcttttaacaattaccaccgtggaggananagcaaggaagagaaatgctgtgatcttttnctgtttttaattagngaaagagggattanattaaacaaatgttacagagntgtgactntgatcccccagnggtaagcaataattgtanagactggatttnanaagccctgagagtttattttcaacctatntattatagnncaatcc

SEQ ID NO: 381 - DNA, 80 bp, Homo sapiens
acaaggcttgggggctggactccctctactgcctctggccataccccctcctggagatggggtcaaggcaccaggactga

SEQ ID NO: 382 - DNA, 435 bp, Homo sapiens
tcgcttgtaaagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggagagacagcccacccccgtgtccaccgtgacccctgtccccacactgacctgtgttccctccccgatcatctttcctgttccagagaagtgggctggatgtctccatctctgtctcaacttcatggtgcgctgagctgcaacttcttacttccctaatgaagttaagaacctgaatataaatttgttttctcaaatatttgctatgaagggttgatggattaattaaataagtcaattcctggaagttgagagagcaaataaagacctgagaaccttccaga

SEQ ID NO: 383 - DNA, 571 bp, Homo sapiens
  misc_feature (1)..(1): n is a, c, g, or t
ngatatagtnccgcatgggaaagatgancaggtataaccnagcntnatatagcaaggactaacccccctgccttctgcataatgaattaactagaaataacttngcaaggagagccaaagctaagaccccngaaaccagacgagctacctaagaacagntaaaagagcacacccgtctatgtagcaaaatagtgggaagatttataggtagaggcgacaaacctaccgagcctggtgatagctggttgtccaagatagaatcttagttcaactttaaattngcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctnacacccaattggaccaatctatcaccctatagaagaactaatgttagtataagtaacatgaaaacattctcctccgcataagcctgcn

SEQ ID NO: 384 - DNA, 689 bp, Homo sapiens
  misc_feature (340)..(340): n is a, c, g, or t
gggaggcggaggctgcagtgagctgagatcgtgccacttcattccagcctgggcaacaaagcgaaactctgtctcaaaaaaaaaaaaaaaaaaaatttgttgactgttgtaatttaaagcttgtcattttttatttagtaataacactcattagtgtagtatctatgatgaaccaggttctgcacaaagtaccttatgttcatggcctcatatcgtcttctccaaaactctgcaagataggattcatcaccacttatagggagagatctgaaagtttaaaattgtacccaaggtcacacagctggtaagtgccagagctgggattccgtagggtgttcanagtgcctctcctgccgtaggcttatcacaaaaagtcaaagtttggtcataataaagcctgaagtttggcaggatttaaaaatagtcaccanacttttgagttggagcatcccacctcactgctgttcaccttctgtggcagggagagtcatcatttccatttcagcttgtggaatatcttgtcattaacattctcatgcaaaagccattttatggtgcccaatgaanatggttaagctactgccccaagcctntggaagccttcctaattttggacttgcactatgcaaattgnataatattttctctaccctaagccaaatattttcttcacttttcattcattctac

SEQ ID NO: 385 - DNA, 542 bp, Homo sapiens
  misc_feature (364)..(364): n is a, c, g, or t
cgccgccgcgccgccgtcgctctccaacgccagcgccgcctctcgctcgccgagctccagccgaaggagaaggggggtaagtaaggaggtctctgtaccatggctcgtacaaagcagactgcccgcaaatcgaccggtggtaaagcacccaggaagcaactggctacaaaagccgctcgcaagagtgcgccctctactggaggggtgaagaaacctcatcgttacaggcctggtactgtggcgctccgtgaaattagacgttatcagaagtccactgaacttctgattcgcaaacttcccttccagcgtctggtgcgagaaattgctcaggactttaaaacagatctgcgcttccagagcgcanctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaatccactatgatgggaaacatttcattctc

SEQ ID NO: 386 - DNA, 198 bp, Homo sapiens
  misc_feature (71)..(72): n is a, c, g, or t
gcgcgtcgactttgtttagacattgaatgactttgttaaaggcacaattaatcacattggttgtactctgnngacagccttctttaaaaaaaaaataaacaatttaaaacaaaaaaaaaaaaaaaaaaaaaaaaaaaaaaaaaaaaaaaaaaaaaaaaaaaaaaaaaaaaaaaaaaaaaaaaaaaaaaanttttaacc

SEQ ID NO: 387 - DNA, 198 bp, Homo sapiens; identical to SEQ ID NO: 386
  misc_feature (71)..(72): n is a, c, g, or t

SEQ ID NO: 388 - DNA, 561 bp, Homo sapiens
tgcatgcttgtggattggaaaaactttggagactgattacttttcattatatatgtgtcacagtgaaacagcttttatgtgtcatgtaagattactgcttgcctctctaaggaaggtcgtgactgtttaaatagacgggcaaggtggaaccttttgaaagatgagcttttgaatataagttgtctgctagatcatggtttgtattgaactaacaaggtttgcagatctgctgacttatataaagctttttgattcctactaagctttaagatttaaaaaatgttcaatgttgaaatttctgtggggctctatttttgctttggctttctggtgagagagtgaggaagcattctttccttcactaagtttgtctttcttgtcttctggatagattgattttaagagactaagggaatttacaaactaaagattttagtcatctggtggaaaaggagactttaagattgtttagggctgggcggggtgactcacatctgtaatcccagcactttgggaggccaaggcaggcagaacacttgaaggagttcaagaccagcgtgg

SEQ ID NO: 389 - DNA, 601 bp, Homo sapiens
  misc_feature (5)..(5): n is a, c, g, or t
tttgncggtnttggannnnnanaantttcttccanncntnacntnttggtggnctaaattaanatggntttngngggttcnttnctnnntnnnncatgggananaattnattntcntncnnnttccttnnccctnaanctaccttcccccnattttctcccctnttcntnaattancatcctctccncntanntcnanacnttaatggcaanactatctaatancnannataananctcctgtnnnccacatntcttattnnncgcnncangttncanncccncagagtnaactcatcctcnncnnaanttcatatcgtgnnctntnnncnntngcgcganatattaannanaccngtanntnnnanacannanntnngnaanaanccttctnannttttagcntcnngcnntaacnnnnntcttngtgnnnncncagctttcncnncatnatnctncnncgaantntcanncntctccncttnaatgnnttcccatgnattaantncctcgnnnanagcactatcgtnnnngagnnnattatngncnntttacntcatgtggtccantnncgttngncgcnnnnaatnttcgtnnnncnn

SEQ ID NO: 390 - DNA, 616 bp, Homo sapiens
  misc_feature (21)..(21): n is a, c, g, or t
ggattttagaggaaggcgctnggttacattggagaactggagtggtctggagttccacggtgtagtggaccagaggccacctctcctgggcttctcagtgtctcgccggcggggttcggcctgagctggattgacatagcccttggcggatttaaacaacctaaacattaagcagtacagctgcctcaaacctttgggattttcagaatgactgacactgccgaagctgttccaaagtttgaagagatgtttgctagtagattcacagaaaatgacaaggagtatcaggaatacctgaaacgccctcctgagtctcctccaattgttgaggaatggaatagcanagctggtgggaaccaaagaaacagaggcaatcggttgcaagacaacagacagttcagaggcagggacaacagatgggggtggccaagtgacaatcgatccaatcagtggcatggacgatcctggggtaacaactacccgcaacacagacaagaaccttactatccccagcaatatggacattatggttacaaccagcggcctccttacggttactactgatagaaatgttggcagcttttagtaaaagcatttactctgttaccatgagaaa

SEQ ID NO: 391 - DNA, 407 bp, Homo sapiens
  misc_feature (1)..(1): n is a, c, g, or t
ngactggctcccgaaaagaagggtggcgagaanaaaaagggccgttctgccatggacgaagtggtaacccgcgaatacaccatcaacattnacaagcgcatccatggagtgggcttcaagaancgtgcacctcgggcactcaaagagattcggaaatttgccatgaaggagatgggaactccatatgtgcgcattgacaccaggctcaacaaanctgtctgggccaaaggaataaggaatgtgccataccgaatccgtgtgcggctgtccanaaaacgtaatgaggatgaagattcaccaaataagctntatactttggttacctatgtacctgttaccactttcaaaaatctacagacagtcaatgtggatganaacnaatcgctgatcgtcagatcaaanaaant

SEQ ID NO: 392 - DNA, 503 bp, Homo sapiens
cagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaag

SEQ ID NO: 393 - DNA, 587 bp, Homo sapiens
  misc_feature (562)..(562): n is a, c, g, or t
tgaaaaataaagtttttatgtatattctacatatgtatatgttggtagaaagcaaaaacgctaggtaaaaataaatgtaatacaattttagctatgaaccaaaaaaccatttgtggtgtggatgcaagaaagtctggatgggtgcagagttctccatgtttcacttctgacatttgaaaatacgcagtttgcatttgatacgtcaaatgttatttttaagaaaaccaataaaatcattaaaaccgaaaaggcagttttgcttgtttttaccttagttggagttatctgcaattgccgtattagtgttttaaggaacttgtaagtaagctccttagtcccctttagagctacgaaacatgtcaattttacttttctccagctttttggaatcttatctaaattaccatgtagagttctgcatagcttcaaattctcttagccaatgtggtctgtaagtgtctatcgatgaatttcaccgttaattgccgtagtatactgtcctgtaccggatgtgaagaggagcaactctgcacagtgcactggttgctcccatggtaggaangaatggcttatcaatggtcggattt

SEQ ID NO: 394 - DNA, 650 bp, Homo sapiens
ggaggatggagcagtgagcgggtctgggcggctgctggcagcgccatggagacggtacagctgaggaacccgccgcgccggcagctgaaaaagttggatgaagatagtttaaccaaacaaccagaagaagtatttgatgtcttagagaaacttggagaagggtgagtgtaaagaaactataggtaggtcattgggtcccagtctttttcctgccccagaagaagcagaaggatatgaacctttcagcattgttctaggtggggtggaaggtaaatttacagcttgtgatgtccttcttcgctttactccaatccctattatagacagatttagtgattcctggtctttttaacacgaagaatatctattgttttctcttttgtaggatctgtatgattttatctacttaacagatagcactaattagattaaaattctataagaaactttttaatttgctgttcataatttctgattggtatgcaataactgtttcaatgaaaatcaatgtaatttagtattttaatatttgcacctttgtgaaatatagtaaataaattaagcactatcaccaccttcacagctacttaggagatccacaatcctgggttgggagccagtggatttcctgaaacacagatttgttaatg

SEQ ID NO: 395 - DNA, 502 bp, Homo sapiens
ctcaagtgaatcctggcttcttggaagcgcttgcctagacgagacacagtgcataaaaacaacttttgggggacaggtatgttttcttgcagctgcggttgtaaggtcttggcaagacaagcagtgtggccagaattttgaacttctgatgaatgtgtaatgcaaaggaccttgtacatttttttgtttcaaggtcctcaaaatgagcacatgaagaggttgctgtgaaactttaagtggccctactgcgcagaagcattcagatgtcacttgatgatctgtaagggaacttgctgatttgggaatgtgcttagggaacacacattccttttgacagggtctgtcactgggtgggtgatgaattatacagatgacatgtgcttttttttcttttttcaacctcaatggtattcctacaggaaatggataaccattttaactgtattttttgcagcccgtaccttcttgggaatacaattgtctaactttttatttttggtct

SEQ ID NO: 396 - DNA, 648 bp, Homo sapiens
ccacaataataagagaaaaacaggagcaaaaggatatacaaaaccaccagaaaacaaataacaaagtgacaggagtaagtccttaactggcaataataaccatgaatctaaatggattccatttcccacttaaaagataaagacatgctgaatggataaaaagctgtcacccagttatatgctgcctacaacaaactcacttcacctgtaaacatacatatggatggaaagagaaggcatgggaaaagatactctactcaaatgaaaacaaaaaccaaacaaaggtggctattcttatatgagataatacagacattaaatcaaaaactggaaacaaacacaaagtcattgtataatgatgaattcaattatatcatgatgaattcaattatatcctccttcctgatcaattcagaaaggaggatataatctttttaaatatatatacacccaacaccagagcatataaatatgtaaaggaagataaagggagtcctgtgatcaagaataaatataacaattataaatattttatctaaagtgatagatagactgtaatacaataatagggtggtgacattaacaccccctctcacattggactgatcatctagaagggagaaaaagctttatgattggaaaagccat

SEQ ID NO: 397 - DNA, 705 bp, Homo sapiens
  misc_feature (668)..(668): n is a, c, g, or t
attgtgttggccacccgggaattcgcggccgcgtcgacctacgcacacgagaacatgcctctcgcaaaggatctccttcatccctctccagaagaggagaagaggaaacacaagaagaaacgcctggtgcagagccccaattcctacttcatggatgtgaaatgcccaggtgaggagacggcttgctgtagtggggaaagcactggacctcaacagttggaaaatgttgtagtgttagctgtctcgtatccttgaagctgtgcagcagcttcagtttcttcgcctgtggaaaatattttccctgatactcttaaaatttgaatgtatgagactggcaaagttttgcatcttaggaggagtgattcatttcaccgtgatctctcatcacatttcacatacaacccctacgtttttttgtgttgggaaacaatgtaatggatgatgagttgggcataagtgcaggaaagacgggtgtaatagaggaaaaaaatgttatctgcttttctttcaggatgctataaaatcaccacggtctttagccatgcacaaacggtagttttgtgtgttggctgctccactgtcctctgccagcctacaggaggaaaagcaaggcttacagaaggatgttccttcaggaggaagcagcactaaaagcactctgagtcaanatgagtgggaaaccatctcaataaacacattttggat

SEQ ID NO: 398 - DNA, 622 bp, Homo sapiens
  misc_feature (597)..(597): n is a, c, g, or t
cttttcctcccgctgtcccccacgggaggggactgctctcccccgctgcatcctttctgtgaggtaccttacccacctcagcacctgagagggtgaaatagaattctaacctcgacattcgggaagtgtttttgagaagtctcggtcggtaagggaagtcttccaagtccgtgcagcactaacgtattggcacctgcctcctcttcggccaccccccagatgaggcagctgtgactgtgtcaagggaagccacgactctgaccatagtcttctctcagcttccactgccgtctccacaggaaacccagaagttctgtgaacaagtccatgctgccatcaaggcatttattgcagtgtactatttgcttccaaaggatcaggccctgagaacaatgaccttatttcctacaacagtgtctgggttgcgtgccagcagatgcctcagataccaagagataacaaagctgcagctcttttgatgctgaccaagaatgtggattttgtgaaggatgcacatgaagaaatggagcaggctgtggaagaatgtgacccttactctggcctcttgaatgatactgaggagaacaactctgacanccacaatcatgaggatgatgtgttg

SEQ ID NO: 399 - DNA, 155 bp, Homo sapiens
cgccacttatccagtgaaccactatcacgaaaaaaactctacctctctatactaatctccctacaaatctccttaattataacattcacagccacagaactaatcatattaaaaaaaaaaaaaaaaaaaaaaaaaaaaaaaaaaaaaaaaaaaaa

SEQ ID NO: 400 - DNA, 700 bp, Homo sapiens
  misc_feature (13)..(13): n is a, c, g, or t
cattgtgttggcncccgggaattcgcggccgcgtcgactttttgtgttgtttggagcagaaatactaaagaagattccgggccgagtatccacagaagtggacgcaaggctctcctttgataaagatgcgatggtggccagagccaggcggctcatcgagctctacaaggaagctgggatcagcaaggaccgaattcttataaagctgtcatcaacctgggaaggaattcaggctggaaaggagctcgaggagcagcacggcatccactgcaacatgacgttactcttctccttcgcccaggctgtggcctgtgccgaggcgggtgtgaccctcatctccccatttgttgggcgcatccttgattggcatgtggcaaacaccgacaagaaatcctatgagcccctggaagaccctggggtaaagagtgtcactaaaatctacaactactacaagaagtttagctacaaaaccattgtcatgggcgcctccttccgcaacacgggcgagatcaaagcactggccggctgtgacttcctcaccatctcacccaagctcctgggagagctgctgcaggacaacgccaagctggtgcctgtgctctcagccaaggcggcccaagccagtgacctggaaaaaatccacctggatgagaagtctttccgttggttgcacaacgaggaccagatggctgtggagaag

SEQ ID NO: 401 - DNA, 479 bp, Homo sapiens
cgtggcagccatctccttctcggcatcatggccgccctcagaccccttgtgaagcccaagatcgtcaaaaagagaaccaagaagttcatccggcaccagtcagaccgatatgtcaaaattaagcgtaactggcggaaacccagaggcattgacaacagggttcgtagaagattcaagggccagatcttgatgcccaacattggttatggaagcaacaaaaaaacaaagcacatgctgcccagtggcttccggaagttcctggtccacaacgtcaaggagctggaagtgctgctgatgtgcaacaaatcttactgtgccgagatcgctcacaatgtttcctccaagaaccgcaaagccatcgtggaaagagctgcccaactggccatcagagtcaccaaccccaatgccaggctgcgcagtgaagaaaatgagtaggcagctcatgtgcacgttttctgtttaaataaatgtaaaaactg

SEQ ID NO: 402 - DNA, 628 bp, Homo sapiens
ctttgattacctttgagtattaggttgaaagcttctctgtgcttgattgaacattgtgatgatgttgattgggtcatgtcagatttagacagtgttgtgtttaagataaatgtttaatggctcttagcagtgttcatgcctccccttttcccctgatactttaaaaacagaatatacagaaaaggggagttgggtgaagaatcaccatattctcattaccagagtagtgtctaccagctgttttcacatttttctgtttccttctgtccttggaatcctttttttagatccttgtaatactagtaaagatattccactctgtgttgtaagcatttttccattttgctccatggtcttcataatgccctgtggtcctttattaaggggatgcaccatgtagaggtgaaaggctttccttgacttggccaccatttctgtattttccttagaggaggaggtttccaacatttcttttttagagacagagtctcgttctgacacgcaggcaggagtgcagtggcatgataacagctcactgcagcctcgaactcctgggctcaagttatcctcccacctcagcttcctgagtagctaggactgcaggtgcctgccaccacacccagctaat

SEQ ID NO: 403 - DNA, 494 bp, Homo sapiens
cagccctccgtcacctcttcaccgcaccctcggactgccccaaggcccccgccgccgcctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagaggggaacatgctgagaaactgatgaagctgcagaaccaacgagggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgac

SEQ ID NO: 404 - DNA, 599 bp, Homo sapiens
  misc_feature (28)..(28): n is a, c, g, or t
gggagacaagcccagcctttcggcgagnatacgtctaaccctgtgcaacagccactacattacttcaaactgagatccttccttttgagggagcaagtccttccctttcattttttccagtcttcctccctgtgtattcattctcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccanagctacccgcagagttctcagaatcatgctgagagagctggaggcacccatgccatctcaacctcttccccgcccgttttacaaagggggaggctaaagcccagagacagcttgatcaaaggcacacagcaagtcagggttggagcagtagctggagggaccttgtctcccagctcagggctctttcctccacaccattcaggtctttctttccgaggcccctgtctcagggtgaggtgcttgagtctccaacggcaagggaacaagtacttcttgatacctgggatactgtgcccagag

SEQ ID NO: 405 - DNA, 610 bp, Homo sapiens
gggagacaagcccagcctttcggcgagatacgtctaaccctgtgcaacagccactacattacttcaaactgagatccttccttttgagggagcaagtccttccctttcattttttccagtcttcctccctgtgtattcattctcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccagagctacccgcagagttctcagaatcatgctgagagagctggaggcacccatgccatctcaacctcttccccgcccgttttacaaagggggaggctaaagcccagagacagcttgatcaaaggcacacagcaagtcagggttggagcagtagctggagggaccttgtctcccagctcagggctctttcctccacaccattcaggtctttctttccgaggcccctgtctcagggtgaggtgcttgagtctccaacggcaagggaacaagtacttcttgatacctgggatactgtgcccagagcctcgaggaggt

SEQ ID NO: 406 - DNA, 644 bp, Homo sapiens
  misc_feature (25)..(25): n is a, c, g, or t
gtttaaatttgacaaactaaagctnatnactgctataagagtaataactgctcattttccataactcattcttaaagttttagtaatgtaaaagttatttttttgcagtaagttataatgatagaagcttacatgttttttcatgcctcatctgtttccccttaaaactataattatcagtaaagtcctgtggtatttttcaatttgtaagaaactaggctatatatacattgggaaaaacagccttcatttgtcaatgcactagtgttccaaaggtttctggtaattgtgtgctattgctttttgttgacttgcaaaaaaaaaaaaaaaaaaattactatgacttgnggtagccctgcaaccttcggaagtgcttagcccagtctgaccatacatttatatttanaatgcttaggtaaataaataatatgcctaaacccaatgctataagatactatataatatctcataattttaaaaatcactgttttgtataataataaaacaaggcaggcaagctgttctacaatgactgttggtaagggtgctgaggaagaaaaacaaacaatcttgattcagggatagtgaatagacaaaaaatgtcctaatcaatgaagctgtgtgatgattctgattgacagaga

SEQ ID NO: 407 - DNA, 653 bp, Homo sapiens
  misc_feature (637)..(637): n is a, c, g, or t
gtgcaaagtgttatatccactttcaacaaagagagaagctgaaaagctaacccaatgttaattttggatcacacacattcagtgtagactttaagattttacttctgttggagtagctatattatttctagttaaaaaactctctatatacatatttatttgtttttctacttgtttaatatttttctcttccaattaggaactcaatatggaataaaaaatatttaaatgtattttactcaaacgtgtgtgtatatatgtttgtgtgcatgataaggagagtgagagcaagagtaagagagagagagcacgcatagatggaagcacacatttaatgtctatgaaatgagaaaacattaaggctaagatatttttccttctgaactagcagattgtatcaatggctggtcacttaaattaatcagtttgtaaagatatttaaaaggtatgtctaccttcttgcaattaatttgattatgttctaatggcatggcaagagaaatgaaagaagataactaaaagttaaaagtcgttgcatgtttttgttgcagcatacccttctttcaggctaccgaataaccttgattgacattggattagtagtagaatacctcattggtagagcatatcgcagcanctacactagaaaacat

SEQ ID NO: 408 - DNA, 452 bp, Homo sapiens
gtctggaactccagacctcaggtgatacccctgcctcagcctcccaatgtgctgggattacagctgtgaagccaccgcgcccggctgctgtgatagttgagatgtaaaccaaaaataaaattctaagccacccaatccgactgaatggacccttcctgttgagcaaggacattccaaagtaaactgaaaagaccagcttaggccatgatgggaaggggaggtgtcaacatgcctcattctaccttcctccctctggaatccagacacaactgaccagcattaacattaaaacagagatcttaagctgggcacggtggctcatgcctgtaatcccagcactttgggaggccaaggtgggatcacctgaggtcggaagttcaagaccagcctggccggtatggtgaagccatgtctctactgaaaatgcaaaattggccggacattgtggtgca

SEQ ID NO: 409 - DNA, 900 bp, Homo sapiens
  misc_feature (2)..(2): n is a, c, g, or t
tncnttttttttcccncgggaaagcgcgccattgtgttggtccccgggaattcgcggccgcgtcgacgagaaatggcttgaacccagtaggcagaggttgtagtgagcccagaatnggncacctgcacntttanccntgggtgacaaaantgaaaactttgtctnaaaaaaaaaaaaaaaaaattttaantnaaatnaaaaancctttncnttntttttnaaannggggggggnntttttngggnttngnnntggtaaaaantnnntttttttttttttaggggccnannccccnttttanaaaanccngnttttnaaaaaanttttttncccncnnttnggggggggggnttttnancnntnttngggggggnncccctnttannaccnncaaantttttanttttttgnnnaannncccccttttttnnttttttttgnggggggggggnngcccccnncctttnggggggggggnttnngnaaaannacttttnaaaannaagggnngggggnanatnncccccccnggnttttttttttaaaaantnaannggggggggnnnctnantnggggcncccanngggggnttanaannattttctncccaaacccccngnttttatnnccccccccccccncnnnngaangggnggnccnttttttttatttttnnggngggnaaaaaantttnaaaaannannatnttttttccccccccccccncttttnggnaaanccnnggggggntcctttttnaaannnncccccaaaaaaaantttttttnttntntttttctctnggggnccnnanttntananttttncnccnaaaaaaaangggncccctttttttncnggnnggnncccaaaannttttttttnaaaaaaaaaaaaaa

SEQ ID NO: 410 - DNA, 271 bp, Homo sapiens
  misc_feature (10)..(10): n is a, c, g, or t
gttcgtgacnttcggagctacctgacagagcagagtcaaccaggntctgcccaaagagagtgttaggcctgagcttgagagccctggagagacgtgtgcacaaaatgtgacctgaggccctagtctagcaagaggacatagcaccctcatctgggaatagggaaggcaccttgcagaaaatatgagcaatttgatattaactaacatcttcaatgtgccatagaccttcccacaaagactgtccaataataagagatgcttatctatttta

SEQ ID NO: 411 - DNA, 412 bp, Homo sapiens
  misc_feature (26)..(26): n is a, c, g, or t
gtcgacgcggccgcggtcgctggagncgatcaactctaggctccaactcgttatgaaaagtgggaagtacgtcctggggtacaagcagactctgaagatgatcagacaaggcaaagcgaaattggtcattctcgctaacaactgcccagctttgaggaaatctgaaatagagtactatgctatgttggctaaaactggtgtccatcactacagtggcaataatattgaactgggcacagcatgcggaaaatactacagagtgtgcacactggctatcattgatccaggtgactctgacatcattagaagcatgccagaacagactggtgaaaagtaaaccttttcacctacaaaatttcacctgcaaaccttaaacctgcaaaattttcctttaataaaatttgcttgtttt

SEQ ID NO: 412 - DNA, 460 bp, Homo sapiens
ccgccaacatgggccgcgttcgcaccaaaaccgtgaagaaggcggcccgggtcatcatagaaaagtactacacgcgcctgggcaacgacttccacacgaacaagcgcgtgtgcgaggagatcgccattatccccagcaaaaagctccgcaacaagatagcaggttatgtcacgcatctgatgaagcgaattcagagaggcccagtaagaggtatctccatcaagctgcaggaggaggagagagaaaggagagacaattatgttcctgaggtctcagccttggatcaggagattattgaagtagatcctgacactaaggaaatgctgaagcttttggacttcggcagtctgtccaaccttcaggtcactcagcctacagttgggatgaatttcaaaacgcctcggggacctgtttgaattttttctgtagtgctgtattattttcaataaatctgggacaa

SEQ ID NO: 413 - DNA, 245 bp, Homo sapiens
cagaggtgggaggattgcttcagttcaagagtttgagaccagcctgggtaacatggcgaaaccctgtctttacaaaaaatgcaaacctttgccgcatgtgttggggtgcgcctgtagtcccagcttctcgggaggctgaggtggggggaccacctgagccatggaggttgaggctgcagtgagccgtgataccaccactgtactctagcctgggccatagagtgagacaccctgcctcagaaata

SEQ ID NO: 414 - DNA, 439 bp, Homo sapiens
cccatcccctcgaccgctcgcgtcgcatttggccgcctccctaccgctccaagcccagccctcagccatggcatgccccctggatcaggccattggcctcctcgtggccatcttccacaagtactccggcagggagggtgacaagcacaccctgagcaagaaggagctgaaggagctgatccagaaggagctcaccattggctcgaagctgcaggatgctgaaattgcaaggctgatggaagacttggaccggaacaaggaccaggaggtgaacttccaggagtatgtcaccttcctgggggccttggctttgatctacaatgaagccctcaagggctgaaaataaatagggaagatggagacaccctctgggggtcctctctgagtcaaatccagtggtgggtaattgtacaataaatttttttttggtcaaatttaa

SEQ ID NO: 415 - DNA, 526 bp, Homo sapiens
  misc_feature (369)..(369): n is a, c, g, or t
ctggagacgacgtgcagaaatggcacctcgaaaggggaaggaaaagaaggaagaacaggtcatcagcctcggacctcaggtggctgaaggagagaatgtatttggtgtctgccatatctttgcatccttcaatgacacttttgtccatgtcactgatctttctggcaaggaaaccatctgccgtgtgactggtgggatgaaggtaaaggcagaccgagatgaatcctcaccatatgctgctatgttggctgcccaggatgtggcccagaggtgcaaggagctgggtatcaccgccctacacatcaaactccgggccacaggaggaaataggaccaagacccctggacctggggcccagtcggccctcanagcccttgcccgctcgggtatgaagatcgggcggattgaggatgtcacccccatcccctctgacagcactcgcaggaaggggggtcgccgtggtcgccgtctgtgaacaagattcctcaaaatattttctgttaataaattgccttcatgtaaactg

SEQ ID NO: 416 - DNA, 613 bp, Homo sapiens
  misc_feature (23)..(23): n is a, c, g, or t
cttaagtatgccctgacaggagnatgaagtaaagaagatttgcatgcagcggttcattaaaatcgatggcaaggtccgaactgatataacctaccctgctggattcatggatgtcatcagcattgacaagacgggagagaatttccgtctgatctatgacaccaagggtcgctttgctgtacatcgtattacacctgaggaggccaagtacaagttgtgcaaagtgagaaagatctttgtgggcacaaaaggaatccctcatctggtgactcatgatgcccgcaccatccgctaccccgatcccctcatcaaggtgaatgataccattcagattgatttagagactggcaagattactgatttcatcaagttcgacactggtaacctgtgtatggtgactggaggtgctaacctaggaagaattggtgtgatcaccaacagagagaggcaccctggatcttttgacgtggttcacgtgaaagatgccaatggcaacagctttgccactcgactttccaacatttttgttattggcaagggcaacaaaccatggatttctcttccccgaggaaagggtatccgcctcaccattgctgaagagagagacaaaaga

SEQ ID NO: 417 - DNA, 663 bp, Homo sapiens
ggaattcgcggccgcgtcgacctctgctcgaattgacagaaaaggattctgtgaagagtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcat

SEQ ID NO: 418 - DNA, 692 bp, Homo sapiens
tgcagaggggtccatacggcgttgttctggattcccgtcgtaacttaaagggaaactttcacaatgtccggagcccttgatgtcctgcaaatgaaggaggaggatgtccttaagttccttgcagcaggaacccacttaggtggcaccaatcttgacttccagatggaacagtacatctataaaaggaaaagtgatggcatctatatcataaatctcaagaggacctgggagaagcttctgctggcagctcgtgcaattgttgccattgaaaaccctgctgatgtcagtgttatatcctccaggaatactggccagagggctgtgctgaagtttgctgctgccactggagccactccaattgctggccgcttcactcctggaaccttcactaaccagatccaggcagccttccgggagccacggcttcttgtggttactgaccccagggctgaccaccagcctctcacggaggcatcttatgttaacctacctaccattgcgctgtgtaacacagattctcctctgcgctatgtggacattgccatcccatgcaacaacaagggagctcactcagtgggtttaatgtggtggatgctggctcgggaagttctgcgcatgcgtggcaccatttcccgtgaacacccatgggaggtcatgcctgatctgtacttctacagagatcctgaagagat

SEQ ID NO: 419 - DNA, 365 bp, Homo sapiens
  misc_feature (214)..(214): n is a, c, g, or t
tttttttttttttcctgcgggaaagcgcgccattgtgttggtacccgggaaattcgcggccgcgtcgacacaggccccagcatcaagatctgggatttagagaggaaagatcattgtagatgaactgaagcaagaagttatcagtaccagcagcaaggcagaaccaccccagtgcacctccctggcctggtctgctgatgacacaggttgggcnggnncncnggggnggnnnngnnnngcngnnggnncngnnnncnnnnngcnnnngnnnntnnncnnngnncnnnnnnnnnnnnnnnnngntcnngnngcnggggccnggncgncgcggncgcgnntnnnngggtncnnncncnnnggcgcgc

SEQ ID NO: 420 - DNA, 299 bp, Homo Sapiens
  misc_feature (242)..(246): n is a, c, g, or t
cagactctgacccagcctcagtcctaactcctggggctgggctgaggggaacaagcatttgctgaaacttgaaaaaacaaagcaaatcaaaaacaggaaaaaattgtacctggtacttttttttagaaaaaaagattaaaaaagaaagaataaattcttgtttggaaacttgaaaaaaaaaaaaaaaaaaaaaaaaaaaaaaaaaaaaaaaaaaaaaaaaaaaaaaaaaattttaaactctnnnnntnncnncnantaanncanntcnannnnannnaattacttnnangtnnntcacn

SEQ ID NO: 421 - DNA, 642 bp, Homo sapiens
  misc_feature (29)..(29): n is a, c, g, or t
acgagaagccagatactaaagagaagaancccgaagccaagaaggttgatgctggtggcaaggtgaaaaagggtaacctcaaagctaaaaagcccaagaaggggaagccccattgcagccgcaaccctgtccttgtcagaggaattggcaggtattcccgatctgccatgtattccanaaaggccatgtacaagaggaagtactcagccgctaaatccaaggttgaaaagaaaaagaaggagaaggttctcgcaactgttacaaaaccagttggtggtgacaagaacggcggtacccgggtggttaaacttcgcaaaatgcctagatattatcctactgaagatgtgcctcgaaagctgttgagccacggcaaaaaacccttcagtcagcacgtgagaaaactgcgagccagcattacccccgggaccattctgatcatcctcactggacgccacaggggcaagagggtggttttcctgaagcagctggctagtggcttattacttgtgactggacctctggtcctcaatcgagttcctctacgaagaacacaccagaaatttgtcattgccacttcaaccaaaatcgatatcagcaatgtaaaaatcccaaaacatcttactgatgcttacttcaaaaaga

SEQ ID NO: 422 - DNA, 503 bp, Homo sapiens
  misc_feature (27)..(27): n is a, c, g, or t
ccctataccttctgcataatgaattanctagaaataactttgcaagggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataag

SEQ ID NO: 423 - DNA, 620 bp, Homo sapiens
ctctcctgtcaacagcggccagcctcccaactacgagaatgctcaaggaggagcaggaagtggctatgctgggggcgccccacaaccctgctcccccgacgtccaccgtgatccacatccgcagcgagacctccgtgcccgaccatgtcgtctggtccctgttcaacaccctcttcatgaacacctgctgcctgggcttcatagcattcgcctactccgtgaagtctagggacaggaagatggttggcgacgtgaccggggcccaggcctatgcctccaccgccaagtgcctgaacatctgggccctgattttgggcatcttcatgaccattctgctcgtcatcatcccagtgttggtcgtccaggcccagcgatagatcaggaggcatcattgaggccaggagctctgcccgtgacctgtatcccacgtactctatcttccattcctcgccctgcccccagaggccaggagctctgcccttgacctgtattccacttactccaccttccattcctcgccctgtccccacagccgagtcctgcatcagccctttatcctcacacgcttttctacaatggcattcaataaagtgtatatgtttctggtgctgctgtgactt

SEQ ID NO: 424 - DNA, 702 bp, Homo sapiens
ttcgtaattagaatactgtttggacttgctcaacaagcaccttatcttaacaaaaagtaacttatagaaaagggagacattcatttaacttcaagcccatattattcttaaaagctgactcttgaaatagtatttattgagtcatagtggagtcatgggactttttaagggccggaagggactatttagatcatccagtcccaccctgtcattttatggaggaggaaactgaggcctagataagataaccagttagtgggtccactgacctttaggacagtagtctatccgtaagagacaacatggagaaagaaatacaacgtttttatagtgaattatcatcttacaaagaatattcttcccatatcgcacttttaaaaagtgggtaccttagtcaaataggagaaaaaaccacttgagtagtttcatcctcaggttttaggtgaggaaactgatactcagattaaataactttaagcacacagagcctgaatgatagtcttatttgagctcatctgtgcttttaatgtgtactacgttaggtgttttcacttgcatttcctttagtcttatttgagctcatctgtgcttttaatgtgtactacgttaggtgttttcacttgcatttccttgtttgacgttgacaataaatcgtgaagctgccttatctaaggaagtcctaaagtaaatcattggaacaca

SEQ ID NO: 425 - DNA, 632 bp, Homo sapiens
  misc_feature (13)..(13): n is a, c, g, or t
ccattgtgttggnacccgggaattcgcggccgcgtcgacggagttttaccttattacactttaatctctggatttaccccatctcatttctcttttaggaaaactgtttgtatgtggtggctttgatggttctcatgccatcagttgtgtggaaatgtatgatccaactagaaatgaatggaagatgatgggaaatatgacttcaccaaggagcaatgctgggattgcaactgtagggaacaccatttatgcagtgggaggattcgatggcaatgaatttctgaatacggtggaagtctataaccttgagtcaaatgaatggagcccctatacaaagattttccagttttaacaaatttaagaccctctcaaactaacaggcttagtgatgtaattatggttagcagaggtacacttgtgaataaagagggtgggtgggtatagatgttgctaacagcaacacaaagcttttgcatattgcatactattaaacatgctgtacatactttttgggtttatttggaaaggaatgcaaagatgaaggtctgttttgtgtacttttaagactttggttattttactttttggaaaagaataaaccaagaattgattgggcacatcatttcaagaag

SEQ ID NO: 426 - DNA, 374 bp, Homo sapiens
agagcagcagccatggccctacgctaccctatggccgtgggcctcaacaagggccacaaagtgaccaagaacgtgagcaagcccaggcacagccgacgccgcgggcgtctgaccaaacacaccaagttcgtgcgggacatgattcgggaggtgtgtggctttgccccgtacgagcggcgcgccatggagttactgaaggtctccaaggacaaacgggccctcaaatttatcaagaaaagggtggggacgcacatccgcgccaagaggaagcgggaggagctgagcaacgtactggccgccatgaggaaagctgctgccaagaaagactgagcccctcccctgccctctccctgaaataaagaacagcttgacag

SEQ ID NO: 427 - DNA, 567 bp, Homo sapiens
gaattattgactttgaattgcatttcagtaccatgaagtcaaagtcagtggtgtatttgctcatttgttcattctttcttttccaccaacattactgcctgcagagccagaggtgagtgcagaaatcctgtcaattcgtcacttgtggacaacctgcagcttgccacagcctacagttccaccactgtgacctctgaaaacctcctgaacaaaaggaaggagacttggaaatcctgaatgggcttggagacattaagggagaactgcctccctggaccaaggcagaattcaatagaaccagcaagaaattttcctatgaatgggaaagcaggtggcagggggcaggggtggaaaagctttgtacaggaattgtggaaaagcttttgcattatctctagtctgaaagtcacatttctcagttcctttccactctcttctgtcaacttgctgtgagtaaatgacatctgtcacctgtgacacgggccagggactatcaccatatggcccccacacattatctagtaccagcctgcctgggccatgccttttccagtcactgtaccagcc

SEQ ID NO: 428 - DNA, 620 bp, Homo sapiens; identical to SEQ ID NO: 423

SEQ ID NO: 429 - DNA, 669 bp, Homo sapiens
cacaagatagaatggtaaaaaaaaaaaaaaaaaaaaaaaaaaaaaaaaaattttaagtgacagtgccatagtttggacagtacctttcaatgattaattttaatagcctgtgagtccaagtaaatgatcactttatttgctagggagggaagtcctagggtggtttcagtttctcccagacatacctaaatttttacatcaatccttttaaagaaaatctgtatttcaaagaatctttctctgcagtaaatctcgcaggggaatttgcactattacacttgaaagttgttattgttaaccttttcggcagcttttaataggaaagttaaacgttttaaacatggtagtactggaaattttacaagacttttacctagcacttaaatatgtataaatgtacataaagacaaactagtaagcatgacctggggaaatggtcagaccttgtattgtgtttttggccttgaaagtagcaagtgaccagaatctgccatggcaacaggctttaaaaaagacccttaaaaagacactgtctcaactgtggtgttagcaccagccagctctctgtacatttgctagcttgtagttttctaagactgagtaaacttcttatttttagaaagtggaggtctggtttgtaactttccttgtacttaattgggtaaaagt

SEQ ID NO: 430 - DNA, 484 bp, Homo sapiens
caaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaatt

SEQ ID NO: 431 - DNA, 576 bp, Homo sapiens
gacaggcgggggcccagcggccgggtgaaggccgggtggctctgtgaatcaaaggagagtcccagaaaacctgtgactgttgaagaaaattcatctgtgaatttttatattcaaggagtcagtatttatattcatcttttaaactgggaagatttatattttactttaaaacttcttgataataatttacaatgaatggacacagtgatgaagaaagtgttagaaacagtagtggagaatcaaggtaagtaagcactttgttatcaattgtttactatgaagagagttgaaaacttgacttttttctttattgttattgttgttatttagttttcctcataggtagcagagttttcaggttttcctcttagctatccaaatactaaaaaaattctgatatacgaaccttttttcataatacaggttttaattatatttttcattcagatacacagtagatcttaaatatagaaagtttttgtttacttaaatctatttggaagtttatatttgagctaataattaagctggagcatgtataatagatttaaattgttttgactgttagtgaaattt

SEQ ID NO: 432 - DNA, 669 bp, Homo sapiens
ctcacttggtgggtgagcctccaatgactacacccaaggaggatttaacacagggattttatgacttgcaacaagtcaggaggacatggggttggggtagttcagcagtgcctgtctgaacaaaggtgaaaattgggcttttattgggctgatcaagggggagtaaaggcagccaggagcagtcgcctgtcatgcttctacctatattgcatgtatagaaaagggaaaataaactccttcctgggcagggttttagtatgctaaggaggggagttattcaacttcaatccaactcaagcatcagcattgctgcgtccatcccagttttgttttgctggggctgaacttcttcctataactttttgaaacaacaagaactcaaggtgtgacagttacaagtgggccctttttcacagtgtgtacctaaacacgtgaggaccctggattacagaatgacagactcgaagtgactcaagttccggttgttcatctttagatggtaaagatggctgtacgtactatccttgcttatttccaatctattgtttaaactcttgtatatgtaataccgcagaggctagagatacaacctttgaccaaatgagtgaattcaagtaatccattactaatgtgatctggaaacaaacatggtgttgaatgtgcatatgt

SEQ ID NO: 433 - DNA, 559 bp, Homo sapiens
  misc_feature (219)..(219): n is a, c, g, or t
cttggcagctccgttatgtgcccagctctttgcaagggcatactgggaaatgagtggagataaaggacccaatcataagcattttacagtatggataccccattttaaaaaggtaaactgaggcacaatgcaatttttttttttttttaaggagtttatttgagcaaacagtgattcatgaatcaggcagcaccaaaccagaaggaggctttgctgaanaaggatgagggacaagcatttataaagtgaatgtagatgtaatacaaagaaaatatttgaaccgggtgcggtggcttacacttgtaatcccaacactttgggaggccaaggcgggcagatcacaagatcaagagatcgagaccatcctggtcaacatggtgaaaccccatctntactaaaaaatacaaaaattanctgggcgtggtggtgcgtgcctgtagtcccagctacttgggcggctgaggcagganaattgcttgaacccgggaggtggaggttgcagtaagccgagattgcaccattgcactactccagcctggtgacagagagagactccatc

SEQ ID NO: 434 - DNA, 1354 bp, Homo sapiens
  misc_feature (3)..(5): n is a, c, g, or t
gannngtgcgatannatgnntgtcttttttttaaagtntttcnnatngnagngaancccccnnanntnncataangagagatnactacngtacanatagngncanacngatagtagtancaanattgtnttagctanatnantcaatagatatcnagatanaanaanancnnggatatacagcgatgtntnannggnnnnnnnanggaacgaacatcnacnttaannataagctngnggagagagacangtangttatanannagaatngnagtaggngtgatcataatagnnnnnanntantatatangatnttantgnnctntnntnngtttatcnnnaatntctatnctngagagnagcnnnatnnnnaggcganganattgggnnntnctcntnatagananctggtgtcnnanaantacntcatctattnanctctcacnanatggnannatanagnagngnnntnnanaggantangcatagngnntnnctnaaacaaaannnataaganntctcgnnaanangggcctntnntntagcgaggnnttantttntatanttnttcnctcttnnaatanntangatanatganctngnngtgatanatannnnntacngtnaanntntantcntataatagatanaaatataggatnttnctctggcnggtngaananttnntncnntttnaataatgntgttagngacngngntntnanannnnnttagaaaggtactctatatactnntatgntncggcnnataatanaacagatgtttgtatnaatatnaaanaaggtcnntttcgncaagagaannntgnctggtnatagaattagcataanttanntantatgatnnantnntnctacnantnttagcnnttngcagnagtcattnngnatntatnnngnntantagtnanttgggnctnntncagantatattntgngaanatgaanntacgnantcctnngnantatnatnntgantanganaancnananntnttntannantgnctatanattgccnngatanattntnnnaatgaancgatagcccgcnctaagganntnngtnanntaaanntctcagataanntacntnttnnttattaancnannatcacantatancngngacannngcganantatatgtatgnnantatnacngntccnnnccgngaanntantcntannaggcattcngnngagctnttctnctagacnatttnnantgaaannatgcngnnaaaaacgacnnncttnaanttntgtctacantccgcnntntttntacagatngcagntaagnnnantnanngctctcanctngctnnnact

SEQ ID NO: 435 - DNA, 741 bp, Homo sapiens
  misc_feature (9)..(9): n is a, c, g, or t
aagcagaantntctctaaaaacattatctccttaaaatcttgaggtgcatatnagagccacaggcaatctctgacatataaaattgcagtacaggcctttcaaatttggcatttcactggtacaatacaacaaccaagatatataataactgtacagtgcctagacattccagtaagaaccattattttctttaatgtagaatgattaatacatattctacaaggggcagtaaggttagtaattctatagggtatgtcccgacataattttcaaattgtacaataacacaaacaactttgttaaggccatgttttatttgctgattaatggacaaaaggcaatgtaatttattttcaagtattttcttgaaagtctgtgctcataaaaatcatgaaaagttggaaagactgttaaatcactgaaacttcaaatatatcttacacaatcttgtttgtacaaaaatacaagttaaatataaacataaagcaatcatggtaattttatgcaaatctgttttatgtgatcatcagttatatataaaagtttctcagttctgttatttgtgaaaagatcaataccagattgaatgactacctattggcaaagggccctaaaaagcttactttagcactcatcttttacatggttaaatgcatttcctaatttgagatcacctaaacactggaaaagaaaaaaaatgaaagggcagtatgtccataaaccaacaaataatttggctg

SEQ ID NO: 436 - DNA, 485 bp, Homo sapiens
cgaaatttccttgtgacacagaggaagggcaaaggtctgagcccagagttgacggagggagtatttcagggttcacttcaggggctcccaaagcgacaagatcgttagggagagaggcccagggtggggactgggaatttaaggagagctgggaacggatcccttaggttcaggaagcttctgtgcaagctgcgaggatggcttgggccgaagggttgctctgcccgccgcgctagctgtgagctgagcaaagccctgggctcacagcaccccaaaagcctgtggcttcagtcctgcgtctgcaccacacaatcaaaaggatcgttttgttttgtttttaaagaaaggtgagattggcttggttcttcatgagcacatttgatatagctctttttctgtttttccttgctcatttcgttttggggaagaaatctgtactgtattgggattgtaaagaacatctctgcactcagacagtttacaga

SEQ ID NO: 437 - DNA, 637 bp, Homo sapiens
  misc_feature (594)..(594): n is a, c, g, or t
ggtttttatacttgccatgaaactgttctttgggatattattttgttcaggttccccacttggacagcagagggggtgactctgcccatccctgccactggtagccaggcgggcaatgtctgctagcagtctgcttctgtctgaactcagccagcagaggcaaactcccggttccccgagaaacactctgaaggcagggtgggtgactccacccaccaccgcctctcctagccatgcaggccatgtctgctagagcttccagcgcagtggtcctaattctgtctgaatccggctgaggggtgcagcctcctgttactgcccagggaaacacccagatggcagggtgggtgactccaaccacctctgcctgtggtagccagatgggccacacctgctagagcttccagcccagcagtcccgctactctgtgggtgggtgccatcccctgttcctctgggaagcacccagacagctgattacgtgaccccacccacttctgcagatcctagctgagcaggacttgctggtttggacaatgcccaagcagggaagagccctcattctcttatcactgacagaggtgagatgtccgantttgtangctggtggaggagtgaggtggaggaggtatgcctct

SEQ ID NO: 438 - DNA, 420 bp, Homo sapiens
gttattcaggtatccatcaaaattttataagagggccggaaacatcggctcacacctgtaatcccagcactttgggaggctgaggcaggtggttcacttgaggtcaggagttcgagaccagcctggccaacatggcaaaaccccgtcactattaaaaatacaaaacattagctgggtgtagtggcaggtgcctgtaatcccagctattcgggaggcctaggaaggaaaatggcttgaacctgggggtggaggttggagtgaggcaagatcacaccactgcactccagcctgggcgacagagcgagactccatctcaaaagaagaaaaaaaaaacaacaaaaaaacctttatcagattatcagaggttatcactacagagggaggtaaaattggagggaaaagggtacaaatttatttcac

SEQ ID NO: 439 - DNA, 741 bp, Homo sapiens; identical to SEQ ID NO: 435
  misc_feature (9)..(9): n is a, c, g, or t

SEQ ID NO: 440 - DNA, 203 bp, Homo sapiens
ttgaggaagggtctactgtctttttaaatggcacaattttaagaggtttgagaggtacagtcccttaacctgccacgggagaggggcccccaaactttcttccccccacacttctggttttctgtgtggagggggagcagggatatctaagctgtggtgtgaaagggtaggagagatgctggaggtgggggtgctgtgttcta

SEQ ID NO: 441 - DNA, 874 bp, Homo sapiens
  misc_feature (134)..(134): n is a, c, g, or t
tttcctcgggaagcgcgccattgtgttggtacccgggaattcgcggccgcgtcgacatttttttttttttttttttttagaatgattaacaatttattgagttttatttatctacaaaaatatagcaatacagngaacttcaccaaatcctaaatattcagtacctgaactggctacaacaccgngtgcacacccagttcctgcagaatctcttgcagatatgggagagtcagccagtgaaaagatccatttcttgggaatccttgtcaacaagaccagttcagaaatccaggatatatagaagcctactgtaatttaaaaacagtaacaaaaaccccaacaaaacccaaatcaacaaagaccaagataaaggngtgataaacattaattgtaatggttttcctttacatgcaatacatgcattttaaaatcactaagaaacacgaaattttgtagagcaaagtttgngtttcacgtaagtgcaaatgaatatatattttattttttatactattaaattatatatattttttccatacaaaagcacacagtgttaatctataaaatgacatccaagtggatgatgattgtttttgcatgtccccctgcttagatttttttaaaatatatagtcaaaaattaacatccttctttaaaaatacagaagggaaaaangggcaaaaaaaaaaatctagactcgagcaagcttatgcatgcatgcggccgcaattcganctcggncgacttggccaattcgccctatagngagtcgnattacaattcactgggccgncgntttacaacgtcgngactgggaaaaccctggcgttacccnnctnatcgncttgnaacaatncccntttngccagngggg

SEQ ID NO: 442 - DNA, 928 bp, Homo sapiens
  misc_feature (12)..(12): n is a, c, g, or t
tcacttcgtatngaanctgtttggacttgctcaacaagaccttatcttaacaaaaagtaacttatagaaaagggagacattcatttaacttcaagcccatattattcttaaaagctgactcttgaaatagtatttattgagtcatagtggagtcatgggactttttaagggccggaagggactatttagatcatccagtcccaccctgtcattttatggaggaggaaactgaggcctagataagataaccagttagtgggtccactgacctttaggacagtagtctatccgtaagagacaacatggagaaagaaatacaacgtttttatagtgaattatcatcttacaaagaatattcttcccatatcgcacttttaaaaagtgggtaccttagtcaaataggagaaaaaaccacttgagtagtttcatcctcaggttttaggtgaggaaactgatactcagattaaataactttaagcacacagagcctgaatgatagtcttatttgagctcatctgtgcttttaatgtgtactacgttaggtgttttcacttgcatttcctttagtcttatttgagctcatctgtgcttttaatgtgtactacgttaggtgttttcacttgcatttccttgtttgacgttgacaataaatcgtgaagctgccttatctaagnagtcctaaagtaaatcattggaacacatgtanccagtttgttgtttttatttgccaggtntcaaatataactgaaaacccatgctaactgactnattttaaaagntgtntggggcatgaaangattgctctgcctgggcgggnggttnancctgngtcccccntttnggagnccacccangangcgatatttnagggnngattcnaaacccctggcacgngnnaaccccntttttaaananaaaanancggnng

SEQ ID NO: 443 - DNA, 954 bp, Homo sapiens
  misc_feature (716)..(716): n is a, c, g, or t
ttgtgttggtacccgggaattcgcggccgcgtcgacggagttttaccttattacactttaatctctggatttaccccatctcatttctcttttaggaaaactgtttgtatgtggtggctttgatggttctcatgccatcagttgtgtggaaatgtatgatccaactagaaatgaatggaagatgatgggaaatatgacttcaccaaggagcaatgctgggattgcaactgtagggaacaccatttatgcagtgggaggattcgatggcaatgaatttctgaatacggtggaagtctataaccttgagtcaaatgaatggagcccctatacaaagattttccagttttaacaaatttaagaccctctcaaactaacaggcttagtgatgtaattatggttagcagaggtacacttgtgaataaagagggtgggtgggtatagatgttgctaacagcaacacaaagcttttgcatattgcatactattaaacatgctgtacatactttttgggtttatttggaaaggaatgcaaagatgaaggtctgttttgtgtacttttaagactttggttattttactttttggaaaagaataaaccaagaattgattgggcacatcatttcaagaagtcccctctcctccacatttgttttgccaatttgcacattaaatgactcttccctcaaatgtgtactatggggtaaaaggggtagggnttaaanatgtaaacagttgggttttttaagggncctttttcataactggaacactctntacaaggntncttnttaaataaataacttgacttttttgttttntaaangnancttcntgcttccataaaaaaaaaaatttaantngncanctntgctgctgcgnccanttngctngnccntggcattccctagggangntnaatantggcnnnttaacnnggcngnaacnnnnnccant

SEQ ID NO: 444 - DNA, 787 bp, Homo sapiens
gggcgatgcatgctttattaaggctcttgtttcacctggcagtgtactgtatcaacgtataatacagaaaaaaaatctctttaaggtcctccttcacaaagacatagagtgaaactccctttacatgtcagtatttgttcaacactttaggcaacttgactgtcagtgttaaaatggaaaacaggaaaatggaaaaatctgaccaattctgccaccttgagactttcatatagaccttgcacaacaattgtatagatcacacaccggctgtatttaatatgtaacattttcacacatattaaagatacagaagtattaaaaaacccccaatgttaatgtatttgcttaaaaggcacaagtttcacatatctgtctagctatctgttggtaatacagaaagtatactacttttttaaaaaagtgggcagaattcttgtgtatgtatatttgtgtgtacagtatgtgtatgtgtgtatatatatatattatatatatagataatatataaatattttttttaaggagaaactagaatgtttagctagaaaattccacagcctgtgaagaaatatttcaaaatggccataaaggaggtaaaaatgaaaaccataacctaacttttatagaggctttatctttaatttaacgatgtgcggaggactttcttgcttgaatctgttccgggctgtctgctctgtccatcaaatgggcaggtctgggaatgaggcaccttcggccgttcagaagtggcctgaacagaatgctggaacccaggctggactcggac

SEQ ID NO: 445 - DNA, 689 bp, Homo sapiens
  misc_feature (673)..(673): n is a, c, g, or t
caaacctgcatgttctgcacatgtatccaggaacttaaaaaaaaaaaaagatagtttgtgtgtcttaattgaataatagtagatttatagattaaagatctatgggtttttaatatggattagaaatctgtgggtttttgatatggattagaaatctgtgggtttttaatatggattggaaatctgtgggtttttaatatggattaaaaaacatctgtgggtttttaatatggattaaacatctgtgggtttttaatatggattaaacatctgggtttttaatatggattaaacatctgtgggtttttaatatgggttaaaaatcaaaagaaaatgaactatttgctccagtgcaggaaaatacaggcaatactggatacaattagatggtcaggagcgataacccggttgccattgtttgaagaagagaataaggtgctagcattcctatccgtagataatttgacagctaggaaatagggggagtcttctatgtagttagtgaaggctaaatgaactattatatgcagttatcgtagaagagtactcaaaaaaatctgtaaaaaataaagaaaggccgggcgcggtggctcacgcctgtaatcccagcactttgggaggccgaggcgggtggatcatgaggtcaggagatcgagaccatcctggctaccanggtgaaacccccgtct

SEQ ID NO: 446 - DNA, 688 bp, Homo sapiens
caagactccatctcaaaaaaaaaaaaaaatctacagtgctgagtatataaaattattaacacatttcacaacaatatgtgtttgtggagttaaatattttttgtctttaaaacaggtaattttagtgcatacttaatttgatgattaaatatggtagaattaagcattttaaatgttaatgtttgttacattgttcaagaaataagtagaaatatattcctttgttttttatttaaatttttgttcctctgtaaactaaaagaacacgaagtaattggtcacaattactggtgtttaactgccaaatatgggtaaataagggaaaattttgtttaatatttagtccttctgagatggcttgaatatttgaattttgttgtacgtctatactgggtagtcacaagtcttataaacactttagaggaaagatggatttcagtctgtatttttaaacatcatttattttaaatctggtgctgaaaaataagaaaaaaattaaactgcattctgctgttcttctttagaagcattcctgcgtaaatactgctgtaatactgtcatgcaaagtgtatcctttcttgtcgtatcctttttggggcagtgtttttttgtttttttcctagaaatgtttgtccttcccccacctgttgatccaggttaaggaatacttttttacactttattcaaa

SEQ ID NO: 447 - DNA, 724 bp, Homo sapiens
cttttcctcccgctgtcccccacggaggggactgctctcccccgctgcatcctttctgtgaggtaccttacccacctcagcacctgagagggtgaaatagaattctaacctcgacattcgggaagtgtttttgagaagtctcggtcggtaagggaagtcttccaagtccgtgcagcactaacgtattggcacctgcctcctcttcggccaccccccagatgaggcagctgtgactgtgtcaagggaagccacgactctgaccatagtcttctctcagcttccactgccgtctccacaggaaacccagaagttctgtgaacaagtccatgctgccatcaaggcatttattgcagtgtactatttgcttccaaaggatcaggccctgagaacaatgaccttatttcctacaacagtgtctgggttgcgtgccagcagatgcctcagataccaagagataacaaagctgcagctcttttgatgctgaccaagaatgtggattttgtgaaggatgcacatgaagaaatggagcaggctgtggaagaatgtgacccttactctggcctcttgaatgatactgaggagaacaactctgacaaccacaatcatgaggatgatgtgttggggtttcccagcaatcaggacttgtattggtcagaggacgatcaagagctcataatcccatgccttgcgctggtgagagcatccaaagcctgcctgaagaaaa

SEQ ID NO: 448 - DNA, 622 bp, Homo sapiens
caagaactctgggacatttgcaaagggtatggcatatgtgtaatgggaataccagaggagaggaaagacaggaagtcaaaaaaagaatttttccaaattaatgataggttccaaaccacagatgcaggaagcttaaacaccaacaggataaataaaacaaaatctacgcttaagcatatcatacttaacctgcagaaaattacagacaaagaaaaaacaccagaggggaagctggcagaaacataccacctatagcggaagaagaataagaattacatcagacttcccttcagaaatcttgcaaacaaaaagatgtagcacaatatttaaagtattaaaggaggccgggcccggtggctcgggcctgtaatcctaacactttgggaggctgaggcaggaggaccatgaggtcaggagatcgagaccatcctggtgatggtgataccccatctctactaaaaatacaaaaaattaaccgggcatggtgacacgcacctgtaatcccagctacttgggaggctgaagcaggagaatcgtttgagcccaggaggtggaggttgcagtgagccgagatcacatcactgcacgcctgggcaacagagcgagactccatctcaaaaaa

SEQ ID NO: 449 - DNA, 834 bp, Homo sapiens
  misc_feature (716)..(716): n is a, c, g, or t
cgacccgttttagtcaggatggtctcgatctcctgacctcgtgatccgcctgcctcggcctcccaaagtgctgggattacaggcgtgagccaccgcgcccggcgtaaatcaggttttttaaatgtttgccaaaccttatcactgacttttataacaaaattatttactataatcattagggaatatttaagttctgctaatacttaaaattgcagagtgctaaaaccagcagtgagtttagaatcaagctaagctttattgttgctactatttgaggcatattagttgactggtgttcatatgcaaggcagtctactgggtgcaacaagggttagaaggatatttttaaaaaactgaccctattctcaggatgaaaataatacactagtaatagtctgctctgttggttaactcctcgtaaggaggtacaattaaaatgctgtagtgttgcaagggaaggagaggaagaatcatattccttcactagcaggatcaagaaagcttttatagaaatatacaaaatcttcacttcttgaaggattggtaaaatttaatagccaacattgggcacttattcattctctgagtaaatatttattgcatgcttatcttgtatcaagcattgtgatgaaagcacaagaatgaaagaggagggagaatgtttagagaataagggctgaaacacagattttgtagggagcgtaggggagactganaagacaggttcaggttagtaagggcgctcatattttgaccctgaatgttaactatgtgcacatcatgctagctattctaaatcaggcattttcaaatggaagcaggcactgacatttt

SEQ ID NO: 450 - DNA, 624 bp, Homo sapiens
cgtgaagggtctttatgtattagtattagagtgatcttttgattattttcctcactataaggaaattatttcctcaggatgagctgccataacattccactgtctgatggcaattttaaagcctgaaattgaagcccatggctaggctatgagaaccctagttcgtatagtaaagttgatatcttctggatgtatactaattttaggctttattttaaaactgctggaaactgaaacttagacaaaagtattttcaggacatcatttacaatgtttagccctaaagagtcaagctgtgggattctgagtctttcatatgttacagcagaaacttaaaagcaagaggaaattggctgggcacagtggctctgtaatcccagcactttgggaggctgaggtgggtggatcatgaggtcaagagattgagaccatcctagccaacatggtgaaaccccatctctactaaaaatacaaaaattagctgggcgtggtggcacacgcctgtaatcccagctagtcaggaggctgaggcaggagaatatcttgaacttgggaggcagaggttgcagtgagccaagattacatcactgcactccagcctggtgacagagcgagactccgact

SEQ ID NO: 451 - DNA, 966 bp, Homo sapiens
ctgaaactgcactgaacccacaggtaggttacatcacaggacagaaatctgaggagctggagaaagcaaaagaataaaggatgggctgacaccagaaggaattaaaggaatttttatactgaacttcaattacttgttcatttgaagtttgtttttttaatgaacgtttttgctgttacttaaatatagtgttttgaaagtgtttcaaatgtattcaagttgggattttccatattttactacagttctgtcttagtatgttcaccataaaacacttatcattaaagctcacaaagtgcttttttgtaatatgaggataaaatgaagccatataagaatttttttatatctgtacatttaacccacatttgagctttagccaaaatatatagcttttttttttctgacctggccaacgtattatccagcaaacatcaactgaagcaatatggaaacacttccaaatgtttgccaataatgctattaagtgactgatgtcaacattagttacatggcaaactaaagaggcattatacatttttaaaacacactaacatataactgtagataatgtaaggtttatttatatgcatatttcatagtatatttaaatgtttaaatataaaaaagggtttttaaacacttttaatttttatctttgattttttttattgatatctctttccaggctactaataaaattgccagaactaaactatcaggtaaaggttaaggcatcaattgacaagtaagttttctaatttcgttttgaattacaattccaaatgtaagacttttaaaaatgaatggcctttattttatagaataattttgaccttttaaatttacttatctaacattatataatgaatgtacttcaaatatttgactttgaagtcaacattaacaaattcatggatcctaattaaaatttactataaaactggaatcatttattacttcctt

SEQ ID NO: 452 - DNA, 725 bp, Homo sapiens
tttttttttttttaaaagagatgggttctcactatgttgcccataatgtttatgagattaagttcatcttttttatctgagtagtattttattgtatgaatataccaccatttatttatctgttggttatttccagttttgggctataatccaaaatgcttttttcaaacaataggctatatatcattaatgtccgtttatcagcagtataaaatatcttaccataaatattaataaaagaagcattcatatataaaatatagatatttcaaaccctacagagggccttttaatgattaaatattttgtccttacaaaaaggtccaggtaattacacccatgaggttaacctgccttagtgcaggacttaaaataaggcttctcctgccatctctctccatttgtagaatgtgaaattctttaaaatgcatcctatattaggaatactatagctgtgcactggtgtttgttctcttctttaaactcgggaccgtatatatctgctcaaattgcccaagtatacatatgctgcactccatcaagtgtcaggccacattctatcagcacagcgtgactgcctatcagtgacaatataagtgagctctatttggatccctcttaccctaccttttatatttatgacagcattatcataaaactccaatattcttcaataacttacatgtttgttgtaggataaaattattaccctcaatgaactacat

SEQ ID NO: 453 - DNA, 715 bp, Homo sapiens
accagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaaaattgatcagggaccatgaaaagaaacttgtgcttcaccgaagaaaaatatctaaacatcgaaaaacttaaatattatggaaaaaaaacattgcaaaatataaaataaataaaaaaaggaaaggaaactttgaaccttatgtaccgagcaaatgccaggtctagcaaacataatgctagtcctagattacttattgatttaaaa

SEQ ID NO: 454 - DNA, 573 bp, Homo sapiens
acattctggaaaaggcaaaagggaggaagaactgattagtggttagcccagggttagagttggggagaggatataatgagggaacttttgtggattctgtaccatgattatgattacacaaacctatgcatacattgaaacacatagaactatacgttgaaaaaagtgaatctgcctgtatgtaaatttaaaagaaaaatatttttttaaaaaaacagatgcttcttaacacattatcatctatgtcagtttaacagttagtagacttaggccaggtgtcatggctcactcctgtaatcccagtgctttgggagtctgaggtgggacgatctcttgagactaggagggagtttgagacaaacctaggcaatgtaatgagactctttctctacaaaaaattttaaagttatctggacatggtggtgcctgcctgtagtcccagctacttgggaggctgaggtgggaggattccttgagcccagaagttcaaggctacagtgtgctatgatagagccactgcactccagcctgggcaaccaggtgagaccttgtctctaaaatgaataaataaat

SEQ ID NO: 455 - DNA, 498 bp, Homo sapiens
tggtctttcacccagccagggagaaggttcttcgctcagtatgaagaaaagcaacccaaaactctcaatctgatttgtttttgtttatgtcgatgccctgtagtttgaaagtgaagtaaagatttagaattcacctaagtccaaaggaaaacacgtggtttttaaagccattaggtaaaaaaagttctcaataaaggcattacaattttttaggtttagaaagatggacttttctgataaatcttggcagacatctaaaaaaaaaaccatatttttcacaagaaaatgcaagttactttttttggaaataatactcactgattatggataaaatggaatattttcagatactatattggctgtttcaaaatagtactattctttaaacttgtaatttttgctaagttatttgtctttgttgtatctataaatatgtaaaaaatatttaaatagatgtacctgttttgctttcacacttaataaaaaatttttttttgt

SEQ ID NO: 456 - DNA, 732 bp, Homo sapiens
cgggatccctagtataacacattcagtgttcccctttcagtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaatgtgaatcagtcactactggaactgcacaaactggccactgacaaaaatgacccccatgtgagtattggaaccccaggaaataaatggaggaaatcatttgccttagggattgggaaagctgcccactaactgtcttccccattgttttgcagttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattccttcaaataaagaaatttggtacc

SEQ ID NO: 457 - DNA, 465 bp, Homo sapiens
tgcgcagaccagacttcgctcgtactcgtgcgcctcgcttcgcttttcctccgcaaccatgtctgacaaacccgatatggctgagatcgagaaattcgataagtcgaaactgaagaagacagagacgcaagagaaaaatccactgccttccaaagaaacgattgaacaggagaagcaagcaggcgaatcgtaatgaggcgtgcgccgccaatatgcactgtacattccacaagcattgccttcttattttacttcttttagctgtttaactttgtaagatgcaaagaggttggatcaagtttaaatgactgtgctgcccctttcacatcaaagaactactgacaacgaaggccgcgcctgcctttcccatctgtctatctatctggctggcagggaaggaaagaacttgcatgttggtgaaggaagaagtggggtggaagaagtggggtgggacgacagtgaaat

SEQ ID NO: 458 - DNA, 788 bp, Homo sapiens
tataaatacactccgggatgatttacccccggaggtcagctagtaaaatacatgagtagaattccttaaagtatgtgataattgctcatcactatccaagtgtgacataaatcataaaaagaattgacaaaatcagggtcgcaaagagaattgaaaaaaatctgtcacaaccaaaatttaaattgacctctgtcctagagtatgagagccacactgaacagaaaaaccagataaatcttttataaaatattcatttgcagccccattaacgttgcttgtcaccccacctccccatgtccttggacaaactgaatgtatagtaacatcatcccaggccaggcgcggtggctcatgcctgtaatcccagcactttgtgaggctaaggcaggcagatcaggaggtcaggagttcaggaccagcctggccaaaaaggtgaaactccgtctctactaacaatacaaaaattagctgggtgcggtagtaggcgcctgtaatcccagctactcgggaggctgaggcaggagaattgctcaaacccggaaggtggaggttgcagtgagctgagatcgtgccactgcactccagcctgggtgacagagcaagactctgtctcggggaggggggtggcggagataaagaaataacatcatcttatactgtcaagctcaaggtgtctgcagccttatcttcaggggaagttgtgtctttctcagggaagatacagatttcaatttagagcaagacagagagaagttacattcagagaggaaaatgcagtagtctaactg

SEQ ID NO: 459 - DNA, 423 bp, Homo sapiens
gcggccgcgctcttttcaatttttaaaaagaagtttgttttccatttcagtaatttctgctttgatcttccttatgtcctcctattgagttgatcagctttctttattcttgccttttctcctctgtgtgccctttctattaacgtatttacccttaggctgggcacaatggctgatgcctgtaatccctgcactttgggaggccgaggcaggtggatcacctaaggtcaggagttcaagaccagcctggccaacatggtgaaacctggtctctactaaaaacacaaaaattagccaggcatggtggtgtgcacctgtaatcccagctactcaggaggctgaggcaggagaattgcttgaacctgggaggcggagattgtgccaaagcactccagcctgggcaacaaaatgagactttgtgtc

SEQ ID NO: 460 - DNA, 231 bp, Homo sapiens
caccaggctgtcttcagatacttcatacagaaatgagcctccctgtggggtcctcttccctccttcagcctgtccatcaacacagcattgcgggatccttaccatggcatccagccctggagatgcttcaggaaagttgcaggtccatgctgcaggacaggctcagatcagcagagacgcatctcacatcgggctgtgaaattcaagttgagctgcaattggcaatgagaa

SEQ ID NO: 461 - DNA, 687 bp, Homo sapiens
gttattcactgagaccgtgccccggttatgaggttgtaccagaaagcaagtattcactatgcacactattcaccgctcaccctagcattgaagccagcctgtagcctgaaagcctttgctttgagggcaggtctttccccaaaatgcagacacgaaggtgcaaagtgaagctgccagtcttgcaaaagatgtaacttgtcacgaaggccacgagtggcagggagagctgtcccacatttgcggaagtggctatgtgaggacgggggaggcgggtcccttagagatgagacaatcataaggggagatatcagagaaaatcgtaaggggagcagatggttgtcaagagaataggctgaccatcgaaggactggcagaagctttcagaaaaccactggacggctgggcacagtggcttaggcctgtaatcccagcactttgggaggctgacgcaggtgaatcacttgaggtcaggagttccagaccagcctggccaacatggtgaaaccccatctctacagaaaatataaaaattagccaggcgtggtggcacaagcctagaatcccagctacttgggaggctgaggcaggcgaatggcttgaacccaggagtcagaggctgcagtgagtcgagattgttccactgcactccagcctgggtgacagtgcaagactccttccaaaaaaaaa

SEQ ID NO: 462 - DNA, 874 bp, Homo sapiens
  misc_feature (717)..(717): n is a, c, g, or t
ttcgtgagtgatggcgtcccgggttgcttgccggtgctggccgccgccgggagagcccggggcagagcagaggtgctcatcagcactgtaggcccggaagattgtgtggtcccgttcctgacccggcctaaggtccctgtcttgcagctggatagcggcaactacctcttctccactagtgcaatctgccgatatttttttttgttatctggctgggagcaagatgacctcactaaccagtggctggaatgggaagcgacagagctgcagccagctttgtctgctgccctgtactatttagtggtccaaggcaagaagggggaagatgttcttggttcagtgcggagagccctgactcacattgaccacagcttgagtcgtcagaactgtcctttcctggctggggagacagaatctctagccgacattgttttgtggggagccctatacccattactgcaagatcccgcctacctccctgaggagctgagtgccctgcacagctggttccagacactgagtacccaggaaccatgtcagcgagctgcagagactgtactgaaacagcaaggtgtcctggctctccggccttacctccaaaagcagccccagcccagccccgctgagggaagggctgtcaccaatgagcctgaggaggaggagctggctaccctatctgaggaggagattgctatggctgttactgcttgggagaanggcctagaaagttttgcccccgctgcggccccagcanaatccagtgttgcctgtggctggagaaaggaatgtgctcatcaccagtgccctccnttacgtcaacaatgtcccccaccttgggaacatcattggttgtgtgctcagtgcccgatgtctt

SEQ ID NO: 463 - DNA, 585 bp, Homo sapiens
cagtgagccaagatcacaccactgcactccagcctggacaacagaacgagactccatatcaaaaaaattaaattaaaatataataaatttcttgccgggcgcagtggctcacacctgtaatcccagcactttgggaggccgaggtgggcggatcacgaagtcaggagattgagaccatcctggctaatacagtgaaaccccgtctctactataaatacaaaaaattagctgggcatggtggcgggcgtctgtagtcccagctactcaggagtctgaggcaggagaatggtgtgaacccgggaggcggagcttgcagtgagccgagatcgtgccactgcaatccagcctgggcagcagaacgagactccatctcaaataaataaataaataaaatgaatttcagctagaagagccttattccattttcctttttattaaacatctggcataagttggtaagtatgtgaagtttatcatatattcttatgcgaattattattttcgcctttttttttataattctgtctgggatttgaatagtagagtttgaattcaggaaggacacctgtgataggacaataaaat

SEQ ID NO: 464 - DNA, 305 bp, Homo sapiens
ctgattgcaaaaacattacaactcagtactgcggctttcattcaaataggttatatgtataaactgaggttcaacaatattgtatttgagatgggaaagttaaagaaatgcaataatgtaaataatacttaagaaaataagatctcaggaaactgtgtatactctgtacttttatgcaactttatcagatcatttcagtatatgcatcaaggatatagtgtatatgacatgaactttgagtgcaaaaactgtactatgtaccttttgtttattttgctgtcaacatctaaataaaggtttttttg

SEQ ID NO: 465 - DNA, 422 bp, Homo sapiens
cgaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagataagatagggaaattagaagataaaaacatacttttagaagaaaaaagataaatttaaacctgaaaagtaggaag

SEQ ID NO: 466 - DNA, 833 bp, Homo sapiens
  misc_feature (7)..(7): n is a, c, g, or t
gtccagnagaaagttcagtgacttgtccagagctgcaggtcttaagaggctgaaatctcgcctctgcctcgaggctgcggttccactgacccatactacttgccttcaggaaagagaaatggtgtaggaaggctgtggatgaagacgcttacattcatgaaggatttggataggcgaacatgagcttttccaccaaatttcagaattttaagaaatgccttaaattatttcttaaaaatcaatttggggcagacgagaagttctgataatagtttttagggaacatgataaaattctgaccttagaagtggtataccagtttgagaagaagaacaagctataaacggtgtagataacattcacggctatttaagaaagagttactaagggaaaccagaatgacttaagagtgttactcttctttttctgagagaacaatagcatcatctcagaaagcctttcatgccattaataggtaagaatctgggcttcttggaccatgggttagactttcttacaaaaccataatatgcatttcctagcaaaatttatgctattacatttccttatctcaacaaagactggtaaattcagtacttattcctcaattttcctacccttaaaatggggatattctgcctctccaaggaatgctgggaacaagcaagtcctcatgttaggggtctttgagttttcatggaagtttaggttatttatatgatgacatagttgtcaacttactttcaggatggacttttcttttgtgagtttgtgacctaaatacaatagttgttatgcatgtccagtttatggaagtaccactgcaatancag

SEQ ID NO: 467 - DNA, 594 bp, Homo sapiens
  misc_feature (54)..(54): n is a, c, g, or t
cagtgcagccaagtatcacaccactgcactccagtcctggacaacagaaacgantactccatatcaaaaaaattaaattaaangataataaatttcttgccgggcgcagtggctcacacctgtaatcccagcactttgggaggccgaggtgggcggatcacgaagtcaggagattgagaccatcctggctaatacagtgaaatccccgtctctactataaatacaaaaaattagctgggcatggtggcgggcgtctgtagtcccagctactcaggagtctgaggcaggagaatggtgtgaacccgggaggcggagcttgcagtgagccgagatcgtgccactgcaatccagcctgggcagcagaacgagactccatctcaaataaataaataaataaaatgaatttcagctagaagagccttattccattttcctttttattaaacatctggcataagttggtaagtatgtgaagtttatcatatattcttatgcgaattattattttcgcctttttttttataattctgtctgggatttgaatagtagagtttgaattcaggaaggacacctgtgataggacaataaaatcta

SEQ ID NO: 468 - DNA, 112 bp, Homo sapiens
gaaagcacatatgatatacatgtgtgtcatatgtattattttgtttgccatctgagtcttcaaaatttgttacagaatacctgcatattaatatttcaaggtatggattaat

SEQ ID NO: 469 - DNA, 40 bp, Homo sapiens
ctgagtattaactaaaaaaaaaaaaaaaaaaaaaaaaaaa

SEQ ID NO: 470 - DNA, 933 bp, Homo sapiens
  misc_feature (740)..(740): n is a, c, g, or t
ccaaacccaactggtccagtaggatactcaccttacagggggcgtctcaagagtctcacagttcccttgggtcttaagagactcactgttggaccaggcgtggtgactcacgcctgtaaaaccagcactttgggaggccgaggcgggcggatcagttgaggtcaagagttcaagaccagcctgaccaaggtgctgaaaccccgtctctactaaaaatacaaaaattagccaggcatggtggtgtgcgcctgtaatcccagctactccagaggctgaggcaggagaatctcttgaacccaggaggtggaggttgcagtgagtcgagatcatgccactgcactccagcctgggtgacagagcgagactccgtcttagaaaaaaaaaaaaaaaaaaaaagaacctcacagttcagcagggttctagcatgagacaatgaggacaagggtaggtgagcaggtggaaagagtgagaacaggtcaattgtgatggagaaaataataaagacagaaaaggcagaagactgcctggcagaagacctgtcccagcagatacaaaaatacagacaacaggagccagcatagacccttgacctgtgtaagtctttctcaggccttcttttaagtagaaacatgcctttgaaaaaaagttttaataaacaggaaaatcataaatccctatttacataaataatatatcctggtcttattcttaaaaccattgatttttcacggctcattaanaaagctgggcgaggtggctcacgcccgtcatcctagcactttgggaggccgaggcgggcanatcacaaggtgaggagttgggagaccagcctgaccaacacggtgaaacccagtctctactaaaaatacaaaaattanctgggggtggtggtgtgtgcctgtaatccaagctactcgggaggctgaggcagga

SEQ ID NO: 471 - DNA, 896 bp, Homo sapiens
cttactacctccaacatgaaacaagcagccccgcacttctcgaaggtctgagttacttggaatcgttttaccacatgatggacagaaggaatatttcagatatctctgaaaacctcaagcgttaccttcttcagtattttaagccagtgattgacaggcaaagctggagtgacaagggctcagtctgggacaggatgctccgctcggctctcttgaagctggcctgtgacctgaaccatgctccttgcatccagaaagctgctgaactcttctcccagtggatggaatccagtggaaaattaaatataccaacagatgttttaaagattgtgtattctgtgggtgctcagacaacagcaggatggaattaccttttagagcaatatgaactgtcaatgtcaagtgctgaacaaaacaaaattctgtatgctttgtcaacgagcaagcatcaggaaaagttactgaagttaattgaactaggaatggaaggaaaggttatcaagacacagaacttggcagctctccttcatgcgattgccagacgtccaaaggggcagcaactagcatgggattttgtaagagaaaattggacccatcttctgaaaaaatttgacttgggctcatatgacataaggatgatcatctctggcacaacagctcacttttcttccaaggataagttgcaagaggtgaaactattttttgaatctcttgaggctcaaggatcacatctggatatttttcaaactgttctggaaacgataaccaaaaatataaaatggctggagaagaatcttccgactctgaggacttggctaatggttaatacttaaatggtcaatagaaaaagtaggctgggcgcggtggctcacgcctgtaatcccagcactttggga

SEQ ID NO: 472 - DNA, 158 bp, Homo sapiens
aaaattttttttttttttttttttttttttttttttttttttttttttttttttttttttttttttttttttttttttttttttttttttttttttttttttttttttcagtgttaaagtaggtttgtcgacgcggccacgaatttcccggggaccaa

SEQ ID NO: 473 - DNA, 896 bp, Homo sapiens
  misc_feature (58)..(58): n is a, c, g, or t
tttttttttttttttgggagtcagttttcttttcttttctttctttttttttttttgnttttcggaaacggagtctcgctttctcgcccactctggagtggngcagtggggnggtctcagctcaccacagcctccacctcctgggcccaagcgatcctntcacctcagcctcctgcgtagctgggactacaggcgtgcaccaccattcccaggtaatttttgtattttttgtananacagggtttcactgttgttgcccaggctggtctcgaactcctgcttcagtctgccanaatgctggattctaggcgtgagccaccgngcctggcccaaaagttacttttcttacagaagcaaagctttaatgcattttactgaatgcttatagctttgtagatactgaaaagagtatgagcgtcacatacagacacatntaacagcactgcctccaaccagcccctacccactggtcaggngagtaanaatcaaaattcttttctgngagtggaacggaaatttcatctctcctcctcaggcaagtagttaanaggctggngggagtcatggccccattttgttcaaaatacaagctccacaggaacaaaaggctgaactgctcacctcccaactgatgaacctcgtctttgttccatgtcaaaggggcctttgtgttactgcagcagaaactccagctatcaaaccatcaggcaccaaaagtaaaactcctttctctaaaaagacctctctttacctgagcctttcaatgcatctttgcccccanataatcctggatgagataatccccagaggaanaccagcgcttgcctagtgaaattatactatgagacaagggtaaaagacctcaaanaccgggttggcaggtaagggagtagggn

SEQ ID NO: 474 - DNA, 350 bp, Homo sapiens
  misc_feature (3)..(3): n is a, c, g, or t
tcngtggcacccgtttccggcaccttcagactctgaagagccacctgcgaatccacacaggagagaaaccttaccatgtacgtaagcctcttgaggccgctctctgacctgcggggatgtggagggcagggaaggaggtggagcgcagggaaggaggtggagcagggaggcagtggaactgtttgctcccatctcaagcacacagtggggcaaccactacgctaatggttggaagacctagatctgggcccaatggccagacaccctgcttgaccttggcccaagcattaggggactcatctttaaaatgagggtatgggactagatgatctgggccttaggagaggagt

SEQ ID NO: 475 - DNA, 835 bp, Homo sapiens
  misc_feature (6)..(6): n is a, c, g, or t
cggctnctaccctgcggagatcacactgacctggcagtgggatggggaggaccaaactcaggacaccgagcttgtggagaccaggccagcaggagatggaaccttccagaagtgggcagctgtggtggtgccttctggagaagagcagagatacacgtgccatgttcagcacgaggggctgccggagcccctcaccctgagatggaagccgtcttcccagcccaccatccccatcgtgggcatcgttgctggcctggctgtcctggctgtcctagctgtcctaggagctatggtggctgttgtgatgtgtaggaggaagagctcaggtggaaaaggagggagctgctctcaggctgcgtccagcaacagtgcccagggctctgatgagtctctcatcgcttgtaaagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggagagacagcccacccccgtgtccaccgtgacccctgtccccacactgacctgtgttccctccccgatcatctttcctgttccagagaagtgggctggatgtctccatctctgtctcaacttcatggtgcgctgagctgcaacttcttacttccctaatgaagttaagaacctgaatataaatttgttttctcaaatatttgctatgaagggttgatggattaattaaataagtcaattcctggaagttgagagagcaaataaagacctgagaaccttccanaatccg

SEQ ID NO: 476 - DNA, 437 bp, Homo sapiens
  misc_feature (18)..(18): n is a, c, g, or t
tgaaacaaaatgaatttntatgggtaagagagggtaatattttagagttgtgttacaaaactacaaatttttattaaattaataaatcagaatactaaatccatgtgtttttttctttcttaaaaaatatcttttggctgggcacggtagctcatggctgtaatcccagcactttgggaggctgaggtgggtggatcgcctgatgtcaggagttcaagaccagcctggtcaacatgttgaaaccccatctctactaaaaatataaaaattagccggtgtggtggtgggcgcctgtaatcccagctactcaggaggctaaggcaggagaattgcgtgaacccaggagttcagtgatgtagcggggagctgagattgtgccactacactccagcctggatgacagagtgagactccatctcaaaaaaaaaaaaaaaaaa

SEQ ID NO: 477 - DNA, 369 bp, Homo sapiens
gcataatgtgaggaggtggagagacagcccacccccgtgtccaccgtgacccctgttcccatgctgacttgtgtttcctccccagtcatctttcctgttccagagaggtggggctggatgtctccatctctgtctcaactttatgtgcactgagctgcaacttcttacttccctactgaaaataagaatctgaatataaatttgttttctcaaatatttgctatgagaggttgatggattaattaaataagtcaattcctggaatttgagagagcaaataaagacctgagaaccttccagaaaaaaaaaaaaaaaaaaaaaaaaaaaaaaaaaaaaaaaaaaaaaaaaaaaaaaaaaaaaaaaaaaaaa

SEQ ID NO: 478 - DNA, 642 bp, Homo sapiens
cttaccatgtcagtgcacagaaatgctgtcttgggatgtaggaaaaataaatccacaaaagctaccaagtttgaaggggaccatgagtcttcaggctggagcttccaaaccagatgaaaaccccacaattaacctgcagtttaagatccagcagctggccatttctggactcaaggtgaatcgtctggatatgtatggagaaaagtacaaaccctttaagggcataaaatacatgaccaaagctgggaagttccaagttcgaacctgaagggagcatttgctgagggaatagtcttgcacattttttcatttcttacttgtctaaaagtaaaaaaaaatatcagcctgtctcctaggtcagtcccctcctggacccacccgctcccttttttccttagccttcagtgccatggaactaatcaagggaggaaaaggtcaccagggagaactggacagaactgaaacacagcaacaccagttctcaaggacaaggtgtgtgatgggggtaggaagcttggtgcttatgtaaccattttaaacgtggtttctataggaaagaccaacatttgtttagcttgcttggctttaattatctaaagccaatgaaagacttctttgttgattttttaagatagaaagatt

SEQ ID NO: 479 - DNA, 912 bp, Homo sapiens
  misc_feature (24)..(24): n is a, c, g, or t
caaacactatgttattttatgaanaagacttgaacatctatggattttggtatttgcaaggggtgaatggggtatttgcaagcagtgaatgaggaggcctggaaccaatcttctgctgatattgaggcacaactgaaaaaggtatattacttaaatctcttattgtattgtaaactgtataagtaatgaaattaaaaggcagaaattgtcagactgaataaaatgaaaagaccaaacaatatgctgcttacaagaaacacaattcaaatataaggacacaattagtttaaaggaaaagaactggaaaagatataccatgataacacaagtcagaagaaagctgctgtggatatattaatatgagatgtagatttcagagcagtgaatattgccaggcataaagaaagttattacataataattaaggtatcagttcatcaagaagatgtaataaccctaagtatttatacaactaatatcagagcttcaaaatacatgaagcaaaaaccagtggaattgataggagaaacacacaattacacaattatagtcagaattttcaacatatctttctcaatggagaaaacaactagacaggaaatcattaaggatatagatgatttaaattatatgatcaactacctggacgtaattggcatttatggaacactgcaccaccaacagcagagtacatattattttcaagtacacagaaaacagttaccaatatagaccattttctgggtcataaaacacatctcaataaatgtaaaacaattaatgttatataaagtatgtgctctgaccncaaaggaattagagatcaataaaagaacatctttgaaaaatctcacntatttaaaaactaataactcacttctaaataactcctgtntcaagagaatnaaangg

SEQ ID NO: 480 - DNA, 873 bp, Homo sapiens
  misc_feature (850)..(850): n is a, c, g, or t
cccagcctcactgcgccccgtcaggccaggcagctgccctcagggtctgccaaggtgggggtcaagggccatgggggcaggtagctctgcctgcaaagcccacaagcatgtcagatcacctgggctgcagacagacaaacacctgagctgttctgaataccttcaggttcctggcctcgctgagcaagtgcagaaatttttaccttcaaggatcagggtttttctgtttgtttgttttttaacacacacatatgtgaacaaagagtatgcgtttgtactggcagaagaagcgtctggtaagacaaccagcaagttaacaatggtcacctccagaaatgggctgggtaaaccaaagaatttttttgtttttgttttttttgagtcagggtctagctctgtcacccaggctggaacgcactggtgtgatcacggctcactgcagccttgacctccctggctcaagcaatcctcccagctcagcctcctgagtcgttgggactacaggcacgtgccaccacgcctgacacattttttaaatttttgtagagacagtgtttcaccatgttgcccaggcaggtctcaaactcctgggctcaagtggtcctccagcttcagcctcccaaagtgctaggattataggtgtgagccacagtgcccagccccgtagtggagaatttctgttgaatgaaccaaaagcaactgccaacctctccatgcaccatgtgtttcagaggagaaagcacagtgaagaatgcagtgtgttctgaggtcctgtcacccctgaggctgtgtgtgtcctttgccaaattaaagagtcttactgaatgcggtgcatccaggagacaggccnaggtttggactggtaaaaaaaaa

SEQ ID NO: 481 - DNA, 778 bp, Homo sapiens
  misc_feature (12)..(12): n is a, c, g, or t
cagacacctggnagaacgggaaggagacgctgcagcgcgcggaccccccaaagacacatgtgacccaccaccccatctntgaccatgaggccaccctgaggtgctgggccctgggcttctaccctgcggagatcacactgacctggcagcgggatggcgaggaccaaactcaggacaccgagcttgtggagaccagaccagcaggagacagaaccttccagaagtgggcagctgtggtggtgccttctggagaagagcagagatacacatgccatgtacagcatgaggggctgccgaagcccctcaccctgagatgggagccatcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtccagcgacagtgcccagggctctgatgtgtctctcacagcttgaaaagcctgagacagctgtnttgtgagggactgagatgcaggatttcttcacgcctcccctttgtgacttcaagagcctctggcatctctttctgcaaaggcacctgaatgtgtctgcgtccttgttagcataatgtgaggaggtggagagacagcccacccttgtgtcaactgtgaccccctgttcccatgctgacctgtgtttcctccccagtcatcttttttgttcncaataggtggggcctggatgtctccatctctgtntca

SEQ ID NO: 482 - DNA, 666 bp, Homo sapiens
ttataaggtacttttaaggtattttagttgtcttagtctatatttctgtactcacctttctttatccactcatcagttgatgggcatgtaggttggttccatatctttgcaattctgaattgtgctgtgatcaggtgtctttttagtataatgatttactctcctttgggtagatacccagtagtgggattgctggatcgaatggtttttataattttctattttaccacagtttctctctgcatttttcctctttgaccactaaccatgtgaaattctcatattgacctttataatgatcatgaactcttagtatcattgggaaggccacatttgccacttatgattgtaaaccttatcctccatttttcctgttattgttggtgcaaaaagcacctattataccaggactttaaaaatcagtctgataagtctttgataagtctaataataataactgataagtccattgaatttgcttctgattactttttctttagtagctaaacatgtatgtactcctatgattacaatgaacactcctctccatttaaattaattatttacattgatgaaatagcaaaatgttaatgactaaatactgtcttggttttttcgttccaggtcagtcaatattaacttcttataattttcttttttttcttt

SEQ ID NO: 483 - DNA, 630 bp, Homo sapiens
gcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggt

SEQ ID NO: 484 - DNA, 612 bp, Homo sapiens
  misc_feature (461)..(461): n is a, c, g, or t
ggccaccgggtgcaaggtcagggctggggtggaggctgggaagcccagggcttggcccactgtggccgccttgtgtggtcactgctttcctgggcctgctgtgagctccctctaggaccccaggcctgtctggtgggtcactgtgaccaccaccttgcacagcacctggcgcgtggcaggtgctcaaacattacttgtttcggaatgaacttcatcttgctcttggctttttgactaatgctgtggaacatctgactaattagtgactctttggggcccccagtttcccagctataaagtggtaatattaagataataattcggccgggcgcggtggctcacgcctgtaatcccagcagcactttgggaggccgaggtgggcagatcacgaggtcagaagatcgagaccatcctggctaacacggtgaaaccccatctctactaaaaatacaaaaaattanccgggcgtggtggcgggcgcctgtagtcccagctactcangaggctgangcaggagaatggtgtgaacccgggaggcagaggttgcagtgaaccaagatcgnnccactgcactccagcctgggcaacagagcgagactccatcttaaaaaa

SEQ ID NO: 485 - DNA, 362 bp, Homo sapiens
aatcagggccgcagtgtgttctgcgcctgcccagagctgactcctgatttaaccgctggcgtaaccgcgggttgcacgcatgcgtgctgaaaagcctttcaccctcacgtggtttcttttttaaccagtcatcaagcgaggctcgcgcgcaggccccgcgttggaaaatggcggggaagctgaaacctctgaatgtggaggcgccagaagctgctgaggaggctgaaggtagtgagggcaagtgggctgcactcctttctctccaaccagggcagaaaggagggaggattcgtcccattacaataatgaaataatgatattctaatttttttaaataaaatgttaagccttttgttattgaa

SEQ ID NO: 486 - DNA, 854 bp, Homo sapiens
  misc_feature (6)..(6): n is a, c, g, or t
ggaaancatgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctntaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttggtaaggactcgggtcggcgccagtcggaggattgggacccccccggatttccccgacagggtcccccanacattccctcaggctggctcttctacgacagccagcctccctcttctggatcagagttttaaatcccanacagaggcttgggactggatgggagagaaggtttgcgaggtgggtccctggggagtcctgttggaggcgtggggccgggaccgcacagggaagtcccgaggcccctctagccccaaaaccanagaaggccttggagacttccctgctgtggcccgaggctnaggaagttttggagttttgggtctgcttanggcttcnagcagccttgcactgagaactttggtagggacctcgagtaatccactccnttttngggactgacgtgaggctcccggtggggaaaganactgacctntc

SEQ ID NO: 487 - DNA, 843 bp, Homo sapiens
ccgacctgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacatttttgtcatataagattcatatttacttcttataca

SEQ ID NO: 488 - DNA, 578 bp, Homo sapiens
taaatagggaatcctttccccattgcttgtttttctcaggtttgtcaaagatcagatagttgtagatatgcgacgttatttctgagggctctgttctgttccattgatctatatctctgtcacatgcacacgtatgtttgttgtggcactattcacagtggcaaagacttggaaccaacccaaatgtccaacaatgatagaccgggttaagaaaatgcggcacatatacaccatggaatactatgtagccataaaaaatgatgagttcgtgtcctttgtagggacatggatgaaattggaaatcatcattctcagtaaactatcgcaggaacaaaaaaccaaacactgcatattctcactcataggtgggaattgaacagtgggaacacatggacacaggaaggggaacatcacactctgaggactgttgtggggtggggggagggaggagggatagcattgggagatatacctagtgctggatgacgagttagtgggtgcagcgcaccagcatgtcacatgtatacatatgtaactaacctgcacattgtgcacatgtaccctaaaacttaaggtat

SEQ ID NO: 489 - DNA, 628 bp, Homo sapiens
  misc_feature (133)..(133): n is a, c, g, or t
ccgcaacaaacacgggagtgcagatatcgctgcgatgggctgatttcctttatttgggtatatacccagcagtgggattgctggattgtatggtagctctattagttttttgaggaacctccaaactgttctncatagtggttgtactcatttacattcccactgtgaaccctgaaaatttgaggcaggtctcagttaaattagaaagttgattttgccaagttggggacacgcactcgtgacacagcctcaggaggaactgatgacatgtgcccaggtggtcagagcacagcttggttttatacattttagggaaacctgagccatcaatcaacatacgtaaaatgggccgggcacagcagctcaagctgtaatcccagcactctgggaggccgaggcgggtggatcacttgaggtcaggagttcgagaccagcctggccaacatggtgaaaccccgtctctattaaaaatacaaagcttagctggatgtggtggcgcatgcctgtagtcccagctgctctaggaggctgaggcatgagaattgcttgaacctgggaggcagaggctgcagtgagccgagatcgagccactatactccagcctggtcaacagagtgagaccctgtct

SEQ ID NO: 490 - DNA, 612 bp, Homo sapiens
ccacaactgtgttcactagcaacctcaaacagacaccatggtgcacctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattg

SEQ ID NO: 491 - DNA, 677 bp, Homo sapiens
  misc_feature (208)..(208): n is a, c, g, or t
atgggcatctctcgggacaactggcacaagcgccgcaaaaccgggggcaagagaaagccctaccacaagaagcggaagtatgagttggggcgcccagctgccaacaccaagattggcccccgccgcatccacacagtccgtgtgcggggaggtaacaagaaataccgtgccctgaggttggacgtggggaatttctcctggggctcanagtgttgtactcgtaaaacaaggatcatcgatgttgtctacaatgcatctaataacgagctggttcgtaccaagaccctggtgaagaattgcatcgtgctcatcgacagcacaccgtaccgacagtggtacgagtcccactatgcgctgcccctgggccgcaagaagggagccaagctgactcctgaggaagaagagattttaaacaaaaaacgatctaaaaaaattcagaagaaatatgatgaaaggaaaaagaatgccaaaatcagcagtctcctggaggagcagttccagcagggcaagcttcttgcgtgcatcgcttcaaggccgggacagtgtggccgagcagatggctatgtgctagagggcaaagagttggagttctatcttaggaaaatcaaggcccgcaaaggcaaataaatccttgttttgtcttcacccatgtaataaaggtgtttattgtttttgtt

SEQ ID NO: 492 - DNA, 736 bp, Homo sapiens
  misc_feature (4)..(4): n is a, c, g, or t
cttncacatactgattgatgtctcatgtctctctaaaatgtgtaaaaccaagctgtgccccaaccaccttgggnacatgtggngaggacctcctgaggctgtgtcatgggcacaccttaaccctgggaaaataaactttctaaactgacttgagagctgtctcagatattctgagcttacagttattgtgaaatcattttaattataaattaagtggagatttacttaaaatcatgtgtagaagtagcctgtgatatagtcctagatacatacattatcatcttatgtatcttccctccctcttccaggttctgataaaaacagatgaaatctgaaagaccatgacagtagtattttgaaaatgacagtatttgaaattaaaaaattgtaaaagtgttctgttctatcactgccaaaggataagttacaaattggttcttggaacgtaatatgtactatgtgcttgctatttaataatttaccagtcttagtcttttttattcagactaattttacctttttttaacctatgactctttagttatagtagtacaaaaaagtagttttagttatagttttagttgtagtacaaaaaagcattttctgtaagcttaatttctttccccttcccgctttcccagtcagatgactttagtgatttggagttgtgtgctttataagtgcattcctcagaggacttaatattactaagattttagcaacnctgaaatatgtt

SEQ ID NO: 493 - DNA, 579 bp, Homo sapiens
  misc_feature (4)..(4): n is a, c, g, or t
tgtncctgtagtcctgtgtgggaggattgcctgagcctaggagctcaaagttgcagtgagcccagatcgngncattgcagtccagcctgggtgacagagtgagaccccatgtcaaaaaaaaaaaaacaaaaaacaggggcctgcctcanccagcaggtgaggtctgccactgagagcacttctagcagcaggaacagcctccacccccacactgcaatcaagttttttgggtcagccttaggagctaanaaagggcctagtttgnctaaatagcaggagttatatccagggatcttcaggcccaggaatgctaatgagtaggcattccatgggccctgggaatggctttgtgtgccanaaatgatggccacaaaggccttgctgccttttttcaaaatggctgcatccagctgagtgctctctgccaaagggganaanaaaataagtctccagtgcatttagattggtctctcatcatctctctcctttttgtttttattagtctccttaaccaaaactgccaagaaaggcttggaattgaaacaaaacctgatanaanaggtaagaggttgttctttt

SEQ ID NO: 494 - DNA, 752 bp, Homo sapiens
  misc_feature (4)..(4): n is a, c, g, or t
tgtntcaaaaaaaaaaaaaagaacggnaatgtactggagatgtatttgataaccaaggntttaggtaaattttcaccagtattagttntatttgcaaactgaaaaatgttgtaggcttaatataaaataaccacattagtgaacattatatctcttagaagaaaggccatattttgctcctgcttctgtaaaaatattatttgtttgaaggggaaataatggtagtgtgacctttcacttaattcctactcccttaatgtgagagagacaaaatgagctgaagaaggaaaattctggagttacactccacaaccttgaacatactgacggacatctctgttttgacaacgatttctccatgccacccatgctntaatgccttgtggatcacggacaaccctctttgcacaagctacagcatcagcgatgttatcttgcagcaaagcactgcaggataaatgacaggcattaactgctcctggggttttgccatcattacaccagtagcggctattgatctgaaatatcccataatcagtgcttctgtctccagcattgtagtttgtagctcgtgtgttgtaaccactctcccatttggccaaacacatccagtttgctaggctgattcccctgtagccatccattcccaatcttttcagagttctggccaactcacacctttcaaagaccttgccctggaccgtaacagaaaggaggacaagccccagaacaatgagagccttcatgttgac

SEQ ID NO: 495 - DNA, 414 bp, Homo sapiens
  misc_feature (103)..(104): n is a, c, g, or t
ttggtacccgggaaattctttgccgcgtcgacggccggtgaggcagatcacctgagcccaggagttcaggaccagcctgggcagcataccgggattccatctnnactaaaaacagtaggctgggtgtggtggctcatgtctgtaagctcaggactttggaaggccaagatgggaggatcacttgagcctgggagtttgacaccagcttgagcatcgtagccaggccctgactctacaaaaaagtgaaataattagccgagtgtggtggttcacacctgtaatcccagctgctcaggaggctgaggtaggagaatcatttgaacccgggaggtggaggttgcagttagccgagatcacgccattgcactccggcctgggcgataaagcgagactctgtctcaaaaaaaaaaaaaa

SEQ ID NO: 496 - DNA, 670 bp, Homo sapiens
attcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaccttagaggtggggagcagagaattctcttatccaacatcaacatcttggtcagatttgaactctt

SEQ ID NO: 497 - DNA, 489 bp, Homo sapiens
ggatttttggtccgcacgctcctgctcctgactcaccgctgttcgctctcgccgaggaacaagtcggtcaggaagcccgcgcgcaacagccatggcttttaaggataccggaaaaacacccgtggagccggaggtggcaattcaccgaattcgaatcaccctaacaagccgcaacgtaaaatccttggaaaaggtgtgtgctgacttgataagaggcgcaaaagaaaagaatctcaaagtgaaaggaccagttcgaatgcctaccaagactttgagaatcactacaagaaaaactccttgtggtgaaggttctaagacgtgggatcgtttccagatgagaattcacaagcgactcattgacttgcacagtccttctgagattgttaagcagattacttccatcagtattgagccaggagttgaggtggaagtcaccattgcagatgcttaagtcaactattttaataaattgatgaccagttgttaaaa

SEQ ID NO: 498 - DNA, 362 bp, Homo sapiens
  misc_feature (307)..(307): n is a, c, g, or t
cttattgaaaattttactaatttcttactttttaggttttaggagaatacttttggataattgactagcctcacattatattgatagaggttcttgaaaactttaatgccaattcatgtatcttatgactaaaatagataatccatttagaaatttaagtcattcttgcgtgcttgatatgtgtcagcactatccaagttgctaggggatacaatggtgaagtgaaaatatcagctaggtgccggtggctcacacctgttatcccaacagtttgggaggccagggtgggaggatcactcaagcacangcgtttcacaccagcctggacaacatacaagaccccatctttaccaaaagttaag

SEQ ID NO: 499 - DNA, 382 bp, Homo sapiens
  misc_feature (193)..(193): n is a, c, g, or t
ttttcttagaactttattttttctggccaggcgcagtggctcacacctgtaatcccagcactttgggaggccaaggcaggtcgatcacctgaggtcaggagctcaagaccagcctggccaacatggtgaaaccctgtctctactaaaaatacaaaaattagctgggcgtggtggcgcatgcctgtaatcccanctactcaggaggctgaggcaggagaattgtttgaacccgggaggcggaggttgcagtgagccgagattgcgccactgcactccagcctgggcaacagagcgaaactccatctcaaaaaaaaaaaaaaaaaacaacctttattttttctgattttaaaagtaataactagtttgtagaaacattaaaagt

SEQ ID NO: 500 - DNA, 556 bp, Homo sapiens
tctttcggaagcgcgccttgtgttggtacccgggaattcgcggccgcgtcgacgcggtcgtaagggctgaggatttttggtccgcacgctcctgctcctgactcaccgctgttcgctctcgccgaggaacaagtcggtcaggaagcccgcgcgcaacagccatggcttttaaggataccggaaaaacacccgtggagccggaggtggcaattcaccgaattcgaatcaccctaacaagccgcaacgtaaaatccttggaaaaggtgtgtgctgacttgataagaggcgcaaaagaaaagaatctcaaagtgaaaggaccagttcgaatgcctaccaagactttgagaatcactacaagaaaaactccttgtggtgaaggttctaagacgtgggatcgtttccagatgagaattcacaagcgactcattgacttgcacagtccttctgagattgttaagcagattacttccatcagtattgagccaggagttgaggtggaagtcaccattgcagatgcttaagtcaactattttaataaattgatgaccagttgttt

SEQ ID NO: 501 - DNA, 464 bp, Homo sapiens
gcggctgctgttggttgggggccgtcccgctcctaaggcaggaagatggtggccgcaaagaagacgaaaaagtcgctggagtcgatcaactctaggctccaactcgttatgaaaagtgggaagtacgtcctggggtacaagcagactctgaagatgatcagacaaggcaaagcgaaattggtcattctcgctaacaactgcccagctttgaggaaatctgaaatagagtactatgctatgttggctaaaactggtgtccatcactacagtggcaataatattgaactgggcacagcatgcggaaaatactacagagtgtgcacactggctatcattgatccaggtgactctgacatcattagaagcatgccagaacagactggtgaaaagtaaaccttttcacctacaaaatttcacctgcaaaccttaaacctgcaaaattttcctttaataaaatttgcttg